SEQUENCE LISTING (1242 sequences)

SEQ ID NO: 1 - protein, 574 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVSKVLHLEGEVNKIKNALQLTNKAVVSLSNGVSVLTSRVLDLKNYINNQLLPMVNRQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAITIVIIVVLLSLIAIGLLLYCKAKNTPVTLSKDQLSGINNIAFSK

SEQ ID NO: 2 - protein, 574 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTCRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAITIVIIVVLLSLIAIGLLLYCKAKNTPVTLSKDQLSGINNIAFSK

SEQ ID NO: 3 - protein, 549 aa, synthetic construct
QNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVSKVLHLEGEVNKIKNALQLTNKAVVSLSNGVSVLTSRVLDLKNYINNQLLPMVNRQSCRISNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAITIVIIVVLLSLIAIGLLLYCKAKNTPVTLSKDQLSGINNIAFSK

SEQ ID NO: 4 - protein, 549 aa, synthetic construct
QNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTCRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITAITIVIIVVLLSLIAIGLLLYCKAKNTPVTLSKDQLSGINNIAFSK

SEQ ID NO: 5 - protein, 488 aa, synthetic construct
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 6 - protein, 494 aa, synthetic construct
  VARIANT 85..111: note = Optionally lacking p27 peptide residues 85-111
QNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSXXXXXXXXXXXXXXXXX

SEQ ID NO: 7 - protein, 494 aa, synthetic construct
  VARIANT 85..111: note = Optionally lacking p27 peptide residues 85-111
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSXXXXXXXXXXXXXXXXX

SEQ ID NO: 8 - protein, 494 aa, synthetic construct
  VARIANT 85..111: note = Optionally lacking p27 peptide residues 85-111
QNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSREIIRAINIVRKIASEK

SEQ ID NO: 9 - protein, 494 aa, synthetic construct
  VARIANT 85..114: note = Optionally lacking p27 peptide residues 85-114
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 10 - protein, 20 aa, synthetic construct
NQSREIIRAINIVRKIASEK

SEQ ID NO: 11 - protein, 20 aa, synthetic construct
NQSALWLEAAKYVKQAREKS

SEQ ID NO: 12 - protein, 20 aa, synthetic construct
NQSAKNAEAAKIAEETKRKD

SEQ ID NO: 13 - protein, 206 aa, synthetic construct
EGMDPLAVLAESRLLPLLTVRGGEDLAGLATVLELMGVGALEITLRTEKGLEALKALRKSGLLLGAGTVRSPKEAEAALEAGAAFLVSPGLLEEVAALAQARGVPYLPGVLTPTEVERALALGLSALKFFPAEPFQGVRVLRAYAEVFPEVRFLPTGGIKEEHLPHYAALPNLLAVGGSWLLQGDLAAVMKKVKAAKALLSPQAPG

SEQ ID NO: 14 - protein, 155 aa, synthetic construct
TKKVGIVDTTFARVDMAEAAIRTLKALSPNIKIIRKTVPGIKDLPVACKKLLEEEGCDIVMALGMPGKAEKDKVCAHEASLGLMLAQLMTNKHIIEVFVHEDEAKDDDELDILALVRAIEHAANVYYLLFKPEYLTRMAGKGLRQGREDAGPARE

SEQ ID NO: 15 - protein, 155 aa, synthetic construct
TKKVGIVDTTFARVDMASAAILTLKMESPNIKIIRKTVPGIKDLPVACKKLLEEEGCDIVMALGMPGKAEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 16 - protein, 208 aa, synthetic construct
STINNQLKALKVIPVIAIDNAEDIIPLGKVLAENGLPAAEITFRSSAAVKAIMLLRSAQPEMLIGAGTILNGVQALAAKEAGATFVVSPGFNPNTVRACQIIGIDIVPGVNNPSTVEAALEMGLTTLKFFPAEASGGISMVKSLVGPYGDIRLMPTGGITPSNIDNYLAIPQVLACGGTWMVDKKLVTNGEWDEIARLTREIVEQVNP

SEQ ID NO: 17 - protein, 113 aa, synthetic construct
PIFTLNTNIKATDVPSDFLSLTSRLVGLILSKPGSYVAVHINTDQQLSFGGSTNPAAFGTLMSIGGIEPSKNRDHSAVLFDHLNAMLGIPKNRMYIHFVNLNGDDVGWNGTTF

SEQ ID NO: 18 - protein, 156 aa, synthetic construct
NQHSHKDYETVRIAVVRARWHADIVDACVEAFEIAMAAIGGDRFAVDVFDVPGAYEIPLHARTLAETGRYGAVLGTAFVVNGGIYRHEFVASAVIDGMMNVQLSTGVPVLSAVLTPHRYRDSAEHHRFFAAHFAVKGVEAARACIEILAAREKIAA

SEQ ID NO: 19 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 20 - protein, 156 aa, synthetic construct
NQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 21 - protein, 176 aa, synthetic construct
FTKSGDDGNTNVINKRVGKDSPLVNFLGDLDELNSFIGFAISKIPWEDMKKDLERVQVELFEIGEDLSTQSSKKKIDESYVLWLLAATAIYRIESGPVKLFVIPGGSEEASVLHVTRSVARRVERNAVKYTKELPEINRMIIVYLNRLSSLLFAMALVANKRRNQSEKIYEIGKSW

SEQ ID NO: 22 - protein, 156 aa, synthetic construct
NQHSHKDYETVRIAVVRARWHADIVDQCVRAFEEAMADAGGDRFAVDVFDVPGAYEIPLHARTLAETGRYGAVLGTAFVVNGGIYRHEFVASAVIDGMMNVQLSTGVPVLSAVLTPHRYRSSREHHEFFREHFMVKGVEAAAACITILAAREKIAA

SEQ ID NO: 23 - protein, 200 aa, synthetic construct
GHTKGPTPQQHDGSALRIGIVHARWNKTIIMPLLIGTIAKLLECGVKASNIVVQSVPGSWELPIAVQRLYSASQLQTPSSGPSLSAGDLLGSSTTDLTALPTTTASSTGPFDALIAIGVLIKGETMHFEYIADSVSHGLMRVQLDTGVPVIFGVLTVLTDDQAKARAGVIEGSHNHGEDWGLAAVEMGVRRRDWAAGKTE

SEQ ID NO: 24 - protein, 236 aa, synthetic construct
YEVDHADVYDLFYLGRGKDYAAEASDIADLVRSRTPEASSLLDVACGTGTHLEHFTKEFGDTAGLELSEDMLTHARKRLPDATLHQGDMRDFQLGRKFSAVVSMFSSVGYLKTVAELGAAVASFAEHLEPGGVVVVEPWWFPETFADGWVSADVVRRDGRTVARVSHSVREGNATRMEVHFTVADPGKGVRHFSDVHLITLFHQREYEAAFMAAGLRVEYLEGGPSGRGLFVGVPA

SEQ ID NO: 25 - protein, 137 aa, synthetic construct
GMKEKFVLIITHGDFGKGLLSGAEVIIGKQENVHTVGLNLGDNIEKVAKEVMRIIIAKLAEDKEIIIVVDLFGGSPFNIALEMMKTFDVKVITGINMPMLVELLTSINVYDTTELLENISKIGKDGIKVIEKSSLKM

SEQ ID NO: 26 - protein, 153 aa, synthetic construct
KYDGSKLRIGILHARWNLEIIAALVAGAIKRLQEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 27 - protein, 163 aa, synthetic construct
AVKGLGEVDQKYDGSKLRIGILHARWNRKIILALVAGAVLRLLEFGVKAENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 28 - protein, 174 aa, synthetic construct
GANWYLDNESSRLSFTSTKNADIAEVHRFLVLHGKVDPKGLAEVEVETESISTGIPLRDMLLRVLVFQVSKFPVAQINAQLDMRPINNLAPGAQLELRLPLTVSLRGKSHSYNAELLATRLDERRFQVVTLEPLVIHAQDFDMVRAFNALRLVAGLSAVSLSVPVGAVLIFTAR

SEQ ID NO: 29 - protein, 207 aa, synthetic construct
TDYIRDGSAIKALSFAIILAEADLRHIPQDLQRLAVRVIHACGMVDVANDLAFSEGAGKAGRNALLAGAPILCDARMVAEGITRSRLPADNRVIYTLSDPSVPELAKKIGNTRSAAALDLWLPHIEGSIVAIGNAPTALFRLFELLDAGAPKPALIIGMPVGFVGAAESKDELAANSRGVPYVIVRGRRGGSAMTAAAVNALASERE

SEQ ID NO: 30 - protein, 127 aa, synthetic construct
ITVFGLKSKLAPRREKLAEVIYSSLHLGLDIPKGKHAIRFLCLEKEDFYYPFDRSDDYTVIEINLMAGRSEETKMLLIFLLFIALERKLGIRAHDVEITIKEQPAHCWGFRGRTGDSARDLDYDIYV

SEQ ID NO: 31 - protein, 234 aa, synthetic construct
GSDLQKLQRFSTCDISDGLLNVYNIPTGGYFPNLTAISPPQNSSIVGTAYTVLFAPIDDPRPAVNYIDSVPPNSILVLALEPHLQSQFHPFIKITQAMYGGLMSTRAQYLKSNGTVVFGRIRDVDEHRTLNHPVFAYGVGSCAPKAVVKAVGTNVQLKILTSDGVTQTICPGDYIAGDNNGIVRIPVQETDISKLVTYIEKSIEVDRLVSEAIKNGLPAKAAQTARRMVLKDYI

SEQ ID NO: 32 - protein, 161 aa, synthetic construct
SGMRVYLGADHAGYELKQAIIAFLKMTGHEPIDCGALRYDADDDYPAFCIAAATRTVADPGSLGIVLGGSGNGEQIAANKVPGARCALAWSVQTAALAREHNNAQLIGIGGRMHTLEEALRIVKAFVTTPWSKAQRHQRRIDILAEYERTHEAPPVPGAPA

SEQ ID NO: 33 - protein, 156 aa, synthetic construct
GDDARIAAIGDVDELNSQIGVLLAEPLPDDVRAALSAIQHDLFDLGGELCIPGHAAITEDHLLRLALWLVHYNGQLPPLEEFILPGGARGAALAHVCRTVCRRAERSIKALGASEPLNIAPAAYVNLLSDLLFVLARVLNRAAGGADVLWDRTRAH

SEQ ID NO: 34 - protein, 156 aa, synthetic construct
ILSAEQSFTLRHPHGQAAALAFVREPAAALAGVQRLRGLDSDGEQVWGELLVRVPLLGEVDLPFRSEIVRTPQGAELRPLTLTGERAWVAVSGQATAAEGGEMAFAFQFQAHLATPEAEGEGGAAFEVMVQAAAGVTLLLVAMALPQGLAAGLPPA

SEQ ID NO: 35 - protein, 155 aa, synthetic construct
TKKVGIVDTTFARVDMASAAILTLKMESPNIKIIRKTVPGIKDLPVACKKLLEEEGCDIVMALGMPGKKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 36 - protein, 208 aa, synthetic construct
DDINNQLKRLKVIPVIAIDNAEDIIPLGKVLAENGLPAAEITFRSSAAVKAIMLLRSAQPEMLIGAGTILNGVQALAAKEAGADFVVSPGFNPNTVRACQIIGIDIVPGVNNPSTVEQALEMGLTTLKFFPAEASGGISMVKSLVGPYGDIRLMPTGGITPDNIDNYLAIPQVLACGGTWMVDKKLVRNGEWDEIARLTREIVEQVNP

SEQ ID NO: 37 - protein, 113 aa, synthetic construct
PIFTLNTNIKADDVPSDFLSLTSRLVGLILSKPGSYVAVHINTDQQLSFGGSTNPAAFGTLMSIGGIEPDKNRDHSAVLFDHLNAMLGIPKNRMYIHFVNLNGDDVGWNGTTF

SEQ ID NO: 38 - protein, 113 aa, synthetic construct
PIFTLNTNIKADDVPSDFLSLTSRLVGLILSEPGSYVAVHINTDQQLSFGGSTNPAAFGTLMSIGGIEPDKNEDHSAVLFDHLNAMLGIPKNRMYIHFVDLDGDDVGWNGTTF

SEQ ID NO: 39 - protein, 156 aa, synthetic construct
NQHSHKDHETVRIAVVRARWHADIVDACVEAFEIAMAAIGGDRFAVDVFDVPGAYEIPLHARTLAETGRYGAVLGTAFVVNGGIYRHEFVASAVIDGMMNVQLDTGVPVLSAVLTPHRYRDSDEHHRFFAAHFAVKGVEAARACIEILNAREKIAA

SEQ ID NO: 40 - protein, 156 aa, synthetic construct
NQHSHKDHETVRIAVVRARWHADIVDACVEAFEIAMAAIGGDRFAVDVFDVPGAYEIPLHARTLAETGRYGAVLGTAFVVDGGIYDHEFVASAVIDGMMNVQLDTGVPVLSAVLTPHEYEDSDEDHEFFAAHFAVKGVEAARACIEILNAREKIAA

SEQ ID NO: 41 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCEWFKAGVLAVGVGDALVKGDPDEVREKAKKFVEKIRGCTE

SEQ ID NO: 42 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPEFVEAMKGPFPNVKFVPTGGVDLDDVCEWFDAGVLAVGVGDALVEGDPDEVREDAKEFVEEIRGCTE

SEQ ID NO: 43 - protein, 202 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHDILKLFPGEVVGPQFVKAMKGPFPNVKFVPTGGVNLDNVCKWFKAGVLAVGVGKALVKGKPDEVREKAKKFVKKIRGCTE

SEQ ID NO: 44 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 45 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 46 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 47 - protein, 156 aa, synthetic construct
TKKVGIVDTTFARVDMASAAILTLKMESPNIKIIRKTVPGIKDLPVACKKLLEEEGCDIVMALGMPGKAKEKDKVCAHEASLGLMLAQLMTNKHIIEVFVHEDEAKDDAELKILAARRAIEHALNVYYLLFKPEYLTRMAGKGLRQGFEDAGPARE

SEQ ID NO: 48 - protein, 215 aa, synthetic construct
SDTDINNQLKARLKVIPVIAIDNAEDIIPLGKVLAENGLPAAEITFRSSAAVKAIMLLRSAQPEMLIGAGTILNGVQALAAKEAGATDFVVSPGFNPNTVRACQIIGIDIVPGVNNPSTVEAQALEMGLTTLKFFPAEASGGISMVKSLVGPYGDIRLMPTGGITPSDNIDNYLAIPQVLACGGTWMVDKKLVTRNGEWDEIARLTREIVEQVNP

SEQ ID NO: 49 - protein, 119 aa, synthetic construct
PIFTLNTNIKATDDVPSDFLSLTSRLVGLILSKEPGSYVAVHINTDQQLSFGGSTNPAAFGTLMSIGGIEPSDKNREDHSAVLFDHLNAMLGIPKNRMYIHFVNDLNDGDDVGWNGTTF

SEQ ID NO: 50 - protein, 166 aa, synthetic construct
NQHSHKDYHETVRIAVVRARWHADIVDACVEAFEIAMAAIGGDRFAVDVFDVPGAYEIPLHARTLAETGRYGAVLGTAFVVNDGGIYRDHEFVASAVIDGMMNVQLSDTGVPVLSAVLTPHREYREDSADEHDHREFFAAHFAVKGVEAARACIEILANAREKIAA

SEQ ID NO: 51 - protein, 219 aa, synthetic construct
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTDILKLFPGEVVGPQEFVKEAMKGPFPNVKFVPTGGVNDLDNDVCEKWFKDAGVLAVGVGSKDALVKEGTDKPDEVREKDAKAEKFVEKKEIRGCTE

SEQ ID NO: 52 - protein, 170 aa, synthetic construct
NQHSHKDYHETVRIAVVRARWHAEIVDACVSAFEAAMARDIGGDRFAVDVFDVPGAYEIPLHARTLAETGRYGAVLGTAFVVNDGGIYRDHEFVASAVIDNGMMNVQLSDNTGVPVLSAVLTPHRENYRDEDKSDKAHDTLLFLALFAVKGMEAARACVEILAAREKIAA

SEQ ID NO: 53 - protein, 158 aa, synthetic construct
GEVPIGDPKELNGMEIAAVYLQPIEMEPRGIDLAASLADIHLEADIHALKNNPNGFPEGFWMPYLTIAYALANADTGAIKTGTLMPMVADDGPHYGANIAMEKDKKGGFGVGTYALTFLISNPEKQGFGRHVDEETGVGKWFEPFVVTYFFKYTGTPK

SEQ ID NO: 54 - protein, 183 aa, synthetic construct
SQAIGILELTSIAKGMELGDAMLKSANVDLLVSKTISPGKFLLMLGGDIGAIQQAIETGTSQAGEMLVDSLVLANIHPSVLPAISGLNSVDKRQAVGIVETWSVAACISAADLAVKGSNVTLVRVHMAFGIGGKCYMVVAGDVLDVAAAVATASLAAGAKGLLVYASIIPRPHEAMWRQMVEG

SEQ ID NO: 55 - protein, 102 aa, synthetic construct
EEVVLITVPSALVAVKIAHALVEERLAACVNIVPGLTSIYRWQGSVVSDHELLLLVKTTTHAFPKLKERVKALHPYTVPEIVALPIAEGNREYLDWLRENTG

SEQ ID NO: 56 - protein, 121 aa, synthetic construct
VRGIRGAITVEEDTPAAILAATIELLLKMLEANGIQSYEELAAVIFTVTEDLTSAFPAEAARLIGMHRVPLLSAREVPVPGSLPRVIRVLALWNTDTPQDRVRHVYLNEAVRLRPDLESAQ

SEQ ID NO: 57 - protein, 176 aa, synthetic construct
SKAKIGIVTVSDRASAGITADISGKAIILALNLYLTSEWEPIYQVIPDEQDVIETTLIKMADEQDCCLIVTTGGTGPAKRDVTPEATEAVCDRMMPGFGELMRAESLKEVPTAILSRQTAGLRGDSLIVNLPGDPASISDCLLAVFPAIPYCIDLMEGPYLECNEAMIKPFRPKAK

SEQ ID NO: 58 - protein, 121 aa, synthetic construct
VRGIRGAITVNSDTPTSIIIATILLLEKMLEANGIQSYEELAAVIFTVTEDLTSAFPAEAARQIGMHRVPLLSAREVPVPGSLPRVIRVLALWNTDTPQDRVRHVYLSEAVRLRPDLESAQ

SEQ ID NO: 59 - protein, 171 aa, synthetic construct
RITTKVGDKGSTRLFGGEEVWKDSPIIEANGTLDELTSFIGEAKHYVDEEMKGILEEIQNDIYKIMGEIGSKGKIEGISEERIAWLLKLILRYMEMVNLKSFVLPGGTLESAKLDVCRTIARRALRKVLTVTREFGIGAEAAAYLLALSDLLFLLARVIEIEKNKLKEVRS

SEQ ID NO: 60 - protein, 122 aa, synthetic construct
PHLVIEATANLRLETSPGELLEQANKALFASGQFGEADIKSRFVTLEAYRQGTAAVERAYLHACLSILDGRDIATRTLLGASLCAVLAEAVAGGGEEGVQVSVEVREMERLSYAKRVVARQR

SEQ ID NO: 61 - protein, 157 aa, synthetic construct
ESVNTSFLSPSLVTIRDFDNGQFAVLRIGRTGFPADKGDIDLCLDKMIGVRAAQIFLGDDTEDGFKGPHIRIRCVDIDDKHTYNAMVYVDLIVGTGASEVERETAEEEAKLALRVALQVDIADEHSCVTQFEMKLREELLSSDSFHPDKDEYYKDFL

SEQ ID NO: 62 - protein, 112 aa, synthetic construct
PVIQTFVSTPLDHHKRLLLAIIYRIVTRVVLGKPEDLVMMTFHDSTPMHFFGSTDPVACVRVEALGGYGPSEPEKVTSIVTAAITAVCGIVADRIFVLYFSPLHCGWNGTNF

SEQ ID NO: 63 - protein, 102 aa, synthetic construct
EEVVLITVPSALVAVKIAHALVEERLAACVNIVPGLTSIYREEGSVVSDHELLLLVKTTTDAFPKLKERVKELHPYEVPEIVALPIAEGNREYLDWLRENTG

SEQ ID NO: 64 - protein, 202 aa, synthetic construct
EELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 65 - protein, 119 aa, synthetic construct
NLAEKMYKAGNAMYRKGQYTIAIIAYTLALLKDPNNAEAWYNLGNAAYKKGEYDEAIEAYQKALELDPNNAEAWYNLGNAYYKQGDYDEAIEYYKKALRLDPRNVDAIENLIEAEEKQG

SEQ ID NO: 66 - protein, 119 aa, synthetic construct
EEAELAYLLGELAYKLGEYRIAIRAYRIALKRDPNNAEAWYNLGNAYYKQGDYREAIRYYLRALKLDPENAEAWYNLGNALYKQGKYDLAIIAYQAALEEDPNNAEAKQNLGNAKQKQG

SEQ ID NO: 67 - protein, 119 aa, synthetic construct
NSAEAMYKMGNAAYKQGDYILAIIAYLLALEKDPNNAEAWYNLGNAAYKQGDYDEAIEYYQKALELDPNNAEAWYNLGNAYYKQGDYDEAIEYYEKALELDPNNAEALKNLLEAIAEQD

SEQ ID NO: 68 - protein, 187 aa, synthetic construct
TDPLAVILYIAILKAEKSIARAKAAEALGKIGDERAVEPLIKALKDEDALVRAAAADALGQIGDERAVEPLIKALKDEEGLVRASAAIALGQIGDERAVQPLIKALTDERDLVRVAAAVALGRIGDEKAVRPLIIVLKDEEGEVREAAAIALGSIGGERVRAAMEKLAERGTGFARKVAVNYLETHK

SEQ ID NO: 69 - protein, 119 aa, synthetic construct
EEAELAYLLGELAYKLGEYRIAIRAYRIALKRDPNNAEAWYNLGNAYYKQGDYDEAIEYYQKALELDPNNAEAWYNLGNAYYKQGDYDEAIEYYEKALELDPENLEALQNLLNAMDKQG

SEQ ID NO: 70 - protein, 279 aa, synthetic construct
IEEVVAEMIDILAESSKKSIEELARAADNKTTEKAVAEAIEEIARLATAAIQLIEALAKNLASEEFMARAISAIAELAKKAIEAIYRLADNHTTDTFMARAIAAIANLAVTAILAIAALASNHTTEEFMARAISAIAELAKKAIEAIYRLADNHTTDKFMAAAIEAIALLATLAILAIALLASNHTTEKFMARAIMAIAILAAKAIEAIYRLADNHTSPTYIEKAIEAIEKIARKAIKAIEMLAKNITTEEYKEKAKKIIDIIRKLAKMAIKKLEDNRT

SEQ ID NO: 71 - protein, 153 aa, synthetic construct
KYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFN

SEQ ID NO: 72 - protein, 119 aa, synthetic construct
EEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 73 - protein, 153 aa, synthetic construct
KYDGSKLRIGILHARGNAEIILALVLGALKRLQEFGVKRENIIIETVPGSFELPYGSKLFVEKQKRLGKPLDAIIPIGVLIRGSTPHFDYIADSTTHQLMKLNFELGIPVIFGVITADTDEQAEARAGLIEGKMHNHGEDWGAAAVEMATKFN

SEQ ID NO: 74 - protein, 150 aa, synthetic construct
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTKAGNHGEDWGAAAVEMATKFN

SEQ ID NO: 75 - protein, 14 aa, synthetic construct
NQSRETAKAVSAVK

SEQ ID NO: 76 - protein, 748 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSARFDASISQVNEAINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 77 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 78 - protein, 748 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSARFAASISQVNEAINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 79 - protein, 748 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSARWAASISQVNEAINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 80 - protein, 748 aa, synthetic construct
MELLILKANAITTILTAVTFCFASGQNITEEFYQSTCSAVSKGYLSALRTGWYTSVITIELSNIKEVKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDRFDASISQVNEAINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 81 - protein, 748 aa, synthetic construct
MELLILKANAITTILTAVTFCFASGQNITEEFYQSTCSAVSKGYLSALRTGWYTSVITIELSNIKEVKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSPYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDRFDASISQVNEAINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 82 - protein, 748 aa, synthetic construct
MELLILKANAITTILTAVTFCFASGQNITEEFYQSTCSAVSKGYLSALRTGWYTSVITIELSNIKEVKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSDRFAASISQVNEAINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 83 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDRFDASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 84 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSPYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDRFDASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 85 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSPYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSDRWAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 86 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSDRFAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 87 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSPYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSDRFAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 88 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSPYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSARFAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 89 - protein, 754 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKDDVSSSVITSLGAIVSCYGKTKCTASNKNRGIIKTFSNGCDYVSNKGVDTVSVGNTLYYVNKQEGKSLYVKGEPIINFYDPLVFPSARWAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 90 - protein, 748 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 91 - protein, 748 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSARFAASISQVNEAINQSLAFIRRSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 92 - protein, 748 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSARWAASISQVNEAINQSLAFIRRSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 93 - protein, 748 aa, synthetic construct
MELLIHRSSAIFLTLAINALYLTSSQNITEEFYQSTCSAVSRGYLSALRTGWYTSVITIELSNIKEVKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDRFDASISQVNEAINQSLAFIRRSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 94 - protein, 748 aa, synthetic construct
MELLIHRSSAIFLTLAINALYLTSSQNITEEFYQSTCSAVSRGYLSALRTGWYTSVITIELSNIKEVKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSPYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDRFDASISQVNEAINQSLAFIRRSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 95 - protein, 748 aa, synthetic construct
MELLIHRSSAIFLTLAINALYLTSSQNITEEFYQSTCSAVSRGYLSALRTGWYTSVITIELSNIKEVKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSDRFAASISQVNEAINQSLAFIRRSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 96 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDRFDASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 97 - protein, 755 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSPYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDRFDASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELLEHHHHHH

SEQ ID NO: 98 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSPYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSDRWAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 99 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSDRFAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 100 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSPYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSDRFAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 101 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSPYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSARFAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 102 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKDDISSSVITSLGAIVSCYGKTKCTASNKNRGIIKTFSNGCDYVSNKGVDTVSVGNTLYYVNKLEGKNLYVKGEPIINYYDPLVFPSARWAASISQVNEAINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 103 - protein, 754 aa, synthetic construct
MELLIHRSSAIFLTLAINALYLTSSQNITEEFYQSTCSAVSRGYLSALRTGWYTSVITIELSNIKETKCNGTDTKVKLIKQELDKYKNAVTELQLLMQNTPAVNNRARREAPQYMNYTINTTKNLNVSISKKRKRRFLGFLLGVGSAIASGIAVCKVLHLEGEVNKIKNALQLTNKAVVSLSNGVSVLTFRVLDLKNYINNQLLPMLNRQSCRISNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSREIIRAINIVRKIASEKGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 104 - protein, 530 aa, synthetic construct
MSWKVVIIFSLLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLESEVTAIKNALKKTNEAVSTLGNGVRVLATAVRELKDFVSKNLTRAINKNKCDIADLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPVKFPEDQFNVALDQVFESIENSQALVDQSNRILSSAEKGNTGFIIVIILTAVLGSTMILVSVFIIIKKTKKPTGAPPELSGV

SEQ ID NO: 105 - protein, 205 aa, synthetic construct
MKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 106 - protein, 205 aa, synthetic construct
MKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVEYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 107 - protein, 205 aa, synthetic construct
MKMEELFKKHKIVAVLRANSVEEAKEKALAVFEGGVHLIEITFTVPDADTVIKELSFLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 108 - protein, 205 aa, synthetic construct; identical to SEQ ID NO: 105

SEQ ID NO: 109 - protein, 205 aa, synthetic construct
MKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 110 - protein, 205 aa, synthetic construct
MKMEELFKKHKIVAVLRANSVEEAKKKALAVFLGGVHLIEITFTVPDADTVIKELSFLKEMGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTE

SEQ ID NO: 111 - protein, 205 aa, synthetic construct
MKMEELFKKHKIVAVLRANSVEEAKMKALAVFVGGVHLIEITFTVPDADTVIKELSFLKELGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTIAEVAAKAAAFVEKIRGCTE

SEQ ID NO: 112 - protein, 205 aa, synthetic construct
MKMEELFKKHKIVAVLRANSVEEAKKKALAVFLGGVDLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPVEVAEKAKAFVEKIRGCTE

SEQ ID NO: 113 - protein, 205 aa, synthetic construct
MKMEELFKKHKIVAVLRANSVEEAKKKALAVFMGGVDLIEITFTVPDADTVIKELSFLKELGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPAEVAEKAKAFVEKIRGCTE

SEQ ID NO: 114 - protein, 167 aa, synthetic construct
MLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKVELIGNENHGLYLADQYVKGIAKSRKS

SEQ ID NO: 115 - protein, 165 aa, synthetic construct
MLKPEMIEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDAQN

SEQ ID NO: 116 - protein, 166 aa, synthetic construct
NFHQDCEAGLNRTVNLKFHSSYVYLSMASYFNRDDVALSNFAKFFRERSEEEKEHAEKLIEYQNQRGGRVFLQSVEKPERDDWANGLEALQTALKLQKSVNQALLDLHAVAADKSDPHMTDFLESPYLSESVETIKKLGDHITSLKKLWSSHPGMAEYLFNKHTLG

SEQ ID NO: 117 - protein, 169 aa, synthetic construct
QFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 118 - protein, 172 aa, synthetic construct
SGESQVRQNFKPEMEEKLNEQMNLELYSSLLYQQMSAWCSYHTFEGAAAFLRRHAQEEMTHMQRLFDYLTDTGNLPRINTVESPFAEYSSLDELFQETYKHEQLITQKINELAHAAMTNQDYPTFNFLQWYVSEQHEEEKLFKSIIDKLSLAGKSGEGLYFIDKELSTLDGS

SEQ ID NO: 119 - protein, 20 aa, synthetic construct
NQSALLLEAAKYVKKAREKS

SEQ ID NO: 120 - protein, 20 aa, synthetic construct
NQSRKLLEAAEEMEKMLKTS

SEQ ID NO: 121 - protein, 20 aa, synthetic construct
NQSRKMLEAVEHAKKLKKES

SEQ ID NO: 122 - protein, 20 aa, synthetic construct
NQSRKMLEAVEKAKKLDKES

SEQ ID NO: 123 - protein, 20 aa, synthetic construct
NQSAKTEEAYQRTIKTQQKL

SEQ ID NO: 124 - protein, 21 aa, synthetic construct
NQSRDLDTAAKQVKEMLKEKS

SEQ ID NO: 125 - protein, 21 aa, synthetic construct
NQSRETEKTIRQVQEILKKWS

SEQ ID NO: 126 - protein, 21 aa, synthetic construct
NQSREVKEAIKIIKKILKKQS

SEQ ID NO: 127 - protein, 21 aa, synthetic construct
NQSREIKDAIKKAKEFIKTIK

SEQ ID NO: 128 - protein, 21 aa, synthetic construct
NQSREIETAIKKAKEFIKTIK

SEQ ID NO: 129 - protein, 19 aa, synthetic construct
NQSRKATETIKKFEESEKS

SEQ ID NO: 130 - protein, 21 aa, synthetic construct
NQSRDTIKVAIIVKELYKKIS

SEQ ID NO: 131 - protein, 22 aa, synthetic construct
NQSRKTLETIEWVKKVIKKQRS

SEQ ID NO: 132 - protein, 22 aa, synthetic construct
NQSRKTLETIEWVEKVIKKQRS

SEQ ID NO: 133 - protein, 18 aa, synthetic construct
NQSRKWNESSKKVQEQDS

SEQ ID NO: 134 - protein, 20 aa, synthetic construct
NQSRKTEKAIRLVLKWLKES

SEQ ID NO: 135 - protein, 23 aa, synthetic construct
NQSRDTLKAIEQTKRYLEELKKS

SEQ ID NO: 136 - protein, 21 aa, synthetic construct
NQSRSWDIAAKFVKTVLSNQS

SEQ ID NO: 137 - protein, 21 aa, synthetic construct
NQSRKTLEATEIAKKLAEDRS

SEQ ID NO: 138 - protein, 23 aa, synthetic construct
NQSLEILKAAKEAKKLIEDLRRS

SEQ ID NO: 139 - protein, 23 aa, synthetic construct
NQSKELLDAAKAVKKMLEKEKSS

SEQ ID NO: 140 - protein, 23 aa, synthetic construct
NQSKKLLDAADAVKKMLEKEKSS

SEQ ID NO: 141 - protein, 23 aa, synthetic construct
NQSKKVLETIRWIETVISRQRSS

SEQ ID NO: 142 - protein, 24 aa, synthetic construct
NQSADLKKVAELVKKLMEEAKKKS

SEQ ID NO: 143 - protein, 21 aa, synthetic construct
NQSTDTMKAARIMKEELKEKS

SEQ ID NO: 144 - protein, 24 aa, synthetic construct
NQSRKTEEALRRADTIIKQLASKS

SEQ ID NO: 145 - protein, 20 aa, synthetic construct
NQSKKLKSAADDVKKAKEKS

SEQ ID NO: 146 - protein, 20 aa, synthetic construct
NQSKELKSAAEDVKKAKEKS

SEQ ID NO: 147 - protein, 22 aa, synthetic construct
NQSRETKKATENVKTMLTKSKS

SEQ ID NO: 148 - protein, 21 aa, synthetic construct
NQSLELKKAAKAANTDLTKKS

SEQ ID NO: 149 - protein, 21 aa, synthetic construct
NQSLELKEAAKAANTDLTKKS

SEQ ID NO: 150 - protein, 24 aa, synthetic construct
NQSRKLEEIARIVEQKKRTEEKRS

SEQ ID NO: 151 - protein, 16 aa, synthetic construct
NQSAETKKAIERAREL

SEQ ID NO: 152 - protein, 16 aa, synthetic construct
NQSRDLKKAAEIAKKS

SEQ ID NO: 153 - protein, 16 aa, synthetic construct
NQSRTLLETAEIVTRS

SEQ ID NO: 154 - protein, 16 aa, synthetic construct
NQSRTLLETAEIVKRS

SEQ ID NO: 155 - protein, 16 aa, synthetic construct
NQSRKLDKAAEYVEKS

SEQ ID NO: 156 - protein, 17 aa, synthetic construct
NQSKEAKKAIETAKKLS

SEQ ID NO: 157 - protein, 17 aa, synthetic construct
NQSRKLETAAEKLKQTE

SEQ ID NO: 158 - protein, 17 aa, synthetic construct
NQSRLMLEAVKIAQSQS

SEQ ID NO: 159 - protein, 18 aa, synthetic construct
NQSRETKEAAESVKQMES

SEQ ID NO: 160 - protein, 18 aa, synthetic construct
NQSRRTLKAIEITLKLLS

SEQ ID NO: 161 - protein, 18 aa, synthetic construct
NQSRRTLTAITRVERKDS

SEQ ID NO: 162 - protein, 19 aa, synthetic construct
NQSKKLADAADWVETVKSS

SEQ ID NO: 163 - protein, 19 aa, synthetic construct
NQSKKTHSAIEWVERLVSS

SEQ ID NO: 164 - protein, 19 aa, synthetic construct
NQSADTKKAAEIAKKLAKS

SEQ ID NO: 165 - protein, 25 aa, synthetic construct
NQSQSIQATTSRVDAIEAKVKHLEA

SEQ ID NO: 166 - protein, 33 aa, synthetic construct
NQSVTINNMISSNTNEISSLQDRVKHIEDTLAL

SEQ ID NO: 167 - protein, 25 aa, synthetic construct
NQSKLVKKVIKETHEIKKKLEDLLK

SEQ ID NO: 168 - protein, 33 aa, synthetic construct
NQSRSNKKTKNKVKSIEKQVKEIEKRLEKLERA

SEQ ID NO: 169 - protein, 27 aa, synthetic construct
NQSQAIRETQDEVKNLNKRINKIVTSI

SEQ ID NO: 170 - protein, 29 aa, synthetic construct
NQSRAIKETQKRTTVLEEDLKRVKELLKS

SEQ ID NO: 171 - protein, 27 aa, synthetic construct
NQSRQIVEVMKEVEELRKRVENIEKNL

SEQ ID NO: 172 - protein, 31 aa, synthetic construct
NQSQKTRATEEALKKTQKEVTKLKKEIQKLT

SEQ ID NO: 173 - protein, 33 aa, synthetic construct
NQSRSNKKTKNKVKSIEKQVKEIEKRLEKLEKA

SEQ ID NO: 174 - protein, 31 aa, synthetic construct
NQSNTVRKTIETVNSLEKELKELRTEVDRLL

SEQ ID NO: 175 - protein, 27 aa, synthetic construct
NQSKEIRNTVKKVRTIEKRLNKLETSL

SEQ ID NO: 176 - protein, 27 aa, synthetic construct
NQSRTLKDTTELTKNLNKKLKKLEEEL

SEQ ID NO: 177 - protein, 29 aa, synthetic construct
NQSKYISNRIKENTDQIKKLEERVTELEA

SEQ ID NO: 178 - protein, 27 aa, synthetic construct
NQSLEIRQTSKRVESLERRVTQVERDR

SEQ ID NO: 179 - protein, 539 aa, synthetic construct
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFESIENSQALVDQSNRILSSAEKGNTGFIIVIILIAVLGSSMILVSIFIIIKKTKKPTGAPPELSGVTNNGFIPHS

SEQ ID NO: 180 - protein, 472 aa, synthetic construct
MSWKVMIIISLLITPQHGLKESYLEESCSTITEGYLSVLRTGWYTNVFTLEVGDVENLTCTDCPSLIKTELDLTKSALRELKTVSADQLAREEQIEGGGGGGFVLGAIALGVATAAAVTAGIAIAKTIRLESEVNAIKGCLKTTNECVSTLGNGVRVLATAVRELKEFVSKNLTSAINKNKCDIADLCMAVSFSQFNRRFLNVVRQFSDNAGITPAISLDLMTDAELARAVSYMPTSAGQIKLMLENRAMVRRKGFGILIGVYGSSVIYMVQLPIFGVIDTPCWIIKAAPSCSEKDGNYACLLREDQGWYCKNAGSTVYYPNDKDCETRGDHVFCDTAAGINVAEQSRECNINISTTNYPCKVSTGRHPISMVALSPLGALVACYKGVSCSIGSNRVGIIKQLPKGCSYITNQDADTVTIDNTVYQLSKVEGEQHVIKGRPVSSSFDPICFPEDQFNVALDQVFESIENCQA

SEQ ID NO: 181 - protein, 472 aa, synthetic construct
MSWKVVIIFSLLITPQHGLKESYLEESCSTITEGYLSVLRTGWYTNVFTLEVGDVENLTCADGPSLIKTELDLTKSALRELRTVSADQLAREEQIEGGGGGGFVLGAIALGVATAAAVTAGVAIAKCIRLESEVTAIKNALKKTNEAVSTLGCGVRVLATAVRELKDFVSKNLTRAINKNKCDIPDLKMAVSFSQFNRRFLNVVRQFSDNAGITPAISKDLMTDAELARAISNMPTSAGQIKLMLENRAMVRRKGFGILIGVYGSSVIYMVQLPIFGVIDTPCWIVKAAPSCSEKKGNYACLLREDQGWYCQNAGSTVYYPNEKDCETRGDHVFCDTAAGINVAEQSKECNINISTTNYPCKVSCGRNPISMVALSPLGALVACYKGVSCSIGSNRVGIIKQLNKGCSYITNQDADTVTIDNTVYQLSKVEGEQHVIKGRPVSSSFDPVKFPEDQFNVALDQCFESIENSQA

SEQ ID NO: 182 - protein, 10 aa, synthetic construct
ENSDRIKRAL

SEQ ID NO: 183 - protein, 10 aa, synthetic construct
ENSSKIKKDL

SEQ ID NO: 184 - protein, 11 aa, synthetic construct
ENSEKLTQAAS

SEQ ID NO: 185 - protein, 11 aa, synthetic construct
ENSDRIKRALS

SEQ ID NO: 186 - protein, 11 aa, synthetic construct
ENSERILSALS

SEQ ID NO: 187 - protein, 11 aa, synthetic construct
ENSEKLAQAVS

SEQ ID NO: 188 - protein, 11 aa, synthetic construct
ENSEILTQQAS

SEQ ID NO: 189 - protein, 11 aa, synthetic construct
ENSERIERAIR

SEQ ID NO: 190 - protein, 11 aa, synthetic construct
ENSDKIKRAIS

SEQ ID NO: 191 - protein, 11 aa, synthetic construct
ENSERIDKAIS

SEQ ID NO: 192 - protein, 11 aa, synthetic construct
ENSEIIKQAIS

SEQ ID NO: 193 - protein, 11 aa, synthetic construct
ENSDRSERAQK

SEQ ID NO: 194 - protein, 12 aa, synthetic construct
ENSTKIEKAITS

SEQ ID NO: 195 - protein, 12 aa, synthetic construct
ENSDRIERASKS

SEQ ID NO: 196 - protein, 12 aa, synthetic construct
ENSETIEKKLQS

SEQ ID NO: 197 - protein, 12 aa, synthetic construct
ENSERIDEAIKR

SEQ ID NO: 198 - protein, 12 aa, synthetic construct
ENSQKILDAIKS

SEQ ID NO: 199 - protein, 12 aa, synthetic construct
ENSERIESAIKS

SEQ ID NO: 200 - protein, 12 aa, synthetic construct
ENSERITKALQS

SEQ ID NO: 201 - protein, 12 aa, synthetic construct
ENSERIEEAIRR

SEQ ID NO: 202 - protein, 13 aa, synthetic construct
ENSEITDRKNKKA

SEQ ID NO: 203 - protein, 13 aa, synthetic construct
ENSDRIKKALSKL

SEQ ID NO: 204 - protein, 13 aa, synthetic construct
ENSEIAKQLMTKA

SEQ ID NO: 205 - protein, 13 aa, synthetic construct
ENSDKIKRAITKT

SEQ ID NO: 206 - protein, 13 aa, synthetic construct
ENSERLERHLRSR

SEQ ID NO: 207 - protein, 13 aa, synthetic construct
ENSQKILDEIKKT

SEQ ID NO: 208 - protein, 13 aa, synthetic construct
ENSESIKEAIKQS

SEQ ID NO: 209 - protein, 13 aa, synthetic construct
ENSIRTKQAIKSA

SEQ ID NO: 210 - protein, 14 aa, synthetic construct
ENSEKIKQTMKKAS

SEQ ID NO: 211 - protein, 14 aa, synthetic construct
ENSSRIKKILSEAS

SEQ ID NO: 212 - protein, 14 aa, synthetic construct
ENSETIKKLLKKAM

SEQ ID NO: 213 - protein, 14 aa, synthetic construct
ENSEKIKQIARLAS

SEQ ID NO: 214 - protein, 14 aa, synthetic construct
ENSETILTTNKRAN

SEQ ID NO: 215 - protein, 14 aa, synthetic construct
ENSQIIQDTIKKMS

SEQ ID NO: 216 - protein, 14 aa, synthetic construct
ENSEKILQAIRLAS

SEQ ID NO: 217 - protein, 14 aa, synthetic construct
ENSEKIEQTRRLAS

SEQ ID NO: 218 - protein, 14 aa, synthetic construct
ENSSRLKKAADKAS

SEQ ID NO: 219 - protein, 14 aa, synthetic construct
ENSTKIAEAIKRTS

SEQ ID NO: 220 - protein, 14 aa, synthetic construct
ENSERINQALKKAD

SEQ ID NO: 221 - protein, 14 aa, synthetic construct
ENSERIKNAIKKME

SEQ ID NO: 222 - protein, 14 aa, synthetic construct
ENSERLDKDAKTAK

SEQ ID NO: 223 - protein, 15 aa, synthetic construct
ENSDKLKRTAEKAKS

SEQ ID NO: 224 - protein, 15 aa, synthetic construct
ENSEEIKTLAKELKE

SEQ ID NO: 225 - protein, 15 aa, synthetic construct
ENSESSKKAQKQAKS

SEQ ID NO: 226 - protein, 15 aa, synthetic construct
ENSEEIKKETKRIRS

SEQ ID NO: 227 - protein, 15 aa, synthetic construct
ENSEKMTKKANTAES

SEQ ID NO: 228 - protein, 15 aa, synthetic construct
ENSEKMTKKANDAES

SEQ ID NO: 229 - protein, 15 aa, synthetic construct
ENSEKIERAIKKAQS

SEQ ID NO: 230 - protein, 15 aa, synthetic construct
ENSEYLAQVAEKVDK

SEQ ID NO: 231 - protein, 15 aa, synthetic construct
ENSEKIERAIKKASS

SEQ ID NO: 232 - protein, 15 aa, synthetic construct
ENSEKIERAIKYALS

SEQ ID NO: 233 - protein, 15 aa, synthetic construct
ENSEKIERAIRKLES

SEQ ID NO: 234 - protein, 15 aa, synthetic construct
ENSERIDSAIKKALS

SEQ ID NO: 235 - protein, 16 aa, synthetic construct
ENSIKIKQQIKRLDEK

SEQ ID NO: 236 - protein, 16 aa, synthetic construct
ENSEKLKRATEKARKS

SEQ ID NO: 237 - protein, 16 aa, synthetic construct
ENSETILRAIKKAQKS

SEQ ID NO: 238 - protein, 16 aa, synthetic construct
ENSEYLLAVAETLNRR

SEQ ID NO: 239 - protein, 16 aa, synthetic construct
ENSEEIDTLAKELKES

SEQ ID NO: 240 - protein, 16 aa, synthetic construct
ENSIKIKTAAKQAKKK

SEQ ID NO: 241 - protein, 16 aa, synthetic construct
ENSERIKETNKATKQK

SEQ ID NO: 242 - protein, 16 aa, synthetic construct
ENSAKIETAIRKTIES

SEQ ID NO: 243 - protein, 16 aa, synthetic construct
ENSEEIKRAIEALRKR

SEQ ID NO: 244 - protein, 16 aa, synthetic construct
ENSSRIKAMIKKILKS

SEQ ID NO: 245 - protein, 16 aa, synthetic construct
ENSEYILTAIKIMLTR

SEQ ID NO: 246 - protein, 17 aa, synthetic construct
ENSEKQKKINEMATKVT

SEQ ID NO: 247 - protein, 17 aa, synthetic construct
ENSERLKKAAEIVERQT

SEQ ID NO: 248 - protein, 17 aa, synthetic construct
ENSETIKKIIEEILSRS

SEQ ID NO: 249 - protein, 17 aa, synthetic construct
ENSEYLKKVAEIVNKIS

SEQ ID NO: 250 - protein, 17 aa, synthetic construct
ENSERTEKAIKITLTIS

SEQ ID NO: 251 - protein, 17 aa, synthetic construct
ENSETLEKVAKEVTKIS

SEQ ID NO: 252 - protein, 17 aa, synthetic construct
ENSDELKRVITDLRKLK

SEQ ID NO: 253 - protein, 17 aa, synthetic construct
ENSTETKKAIEIALKIS

SEQ ID NO: 254 - protein, 17 aa, synthetic construct
ENSEKITKAIEEMKKQS

SEQ ID NO: 255 - protein, 17 aa, synthetic construct
ENSEKLEKAMEETKKLS

SEQ ID NO: 256 - protein, 18 aa, synthetic construct
ENSEKILTAIKIALAAVS

SEQ ID NO: 257 - protein, 18 aa, synthetic construct
ENSERLDKTAKETKEYLS

SEQ ID NO: 258 - protein, 18 aa, synthetic construct
ENSDKIKKAVSWVLAVKS

SEQ ID NO: 259 - protein, 18 aa, synthetic construct
ENSERIKSAIKKLESQES

SEQ ID NO: 260 - protein, 18 aa, synthetic construct
ENSEKIKSALELALRLAK

SEQ ID NO: 261 - protein, 18 aa, synthetic construct
ENSERIEEAIRRASKNDG

SEQ ID NO: 262 - protein, 18 aa, synthetic construct
ENSEKLEKLERKTRQKDS

SEQ ID NO: 263 - protein, 18 aa, synthetic construct
ENSEKIKQAIELTLKLAS

SEQ ID NO: 264 - protein, 18 aa, synthetic construct
ENSEAIERTLKTIDKKVS

SEQ ID NO: 265 - protein, 18 aa, synthetic construct
ENSEELKKVAKEAKKAIS

SEQ ID NO: 266 - protein, 18 aa, synthetic construct
ENSAKIEKTLKKLKTEDS

SEQ ID NO: 267 - protein, 18 aa, synthetic construct
ENSSKLEEALRWVTKVRS

SEQ ID NO: 268 - protein, 18 aa, synthetic construct
ENSARIKKTIEIVLTQTS

SEQ ID NO: 269 - protein, 19 aa, synthetic construct
ENSDRLIKVAEKTSKMLKS

SEQ ID NO: 270 - protein, 19 aa, synthetic construct
ENSQILLDAMTNTERALRS

SEQ ID NO: 271 - protein, 19 aa, synthetic construct
ENSDRLKKMLEKTSKMLKS

SEQ ID NO: 272 - protein, 19 aa, synthetic construct
ENSEKIKRAIDIVEKLTQS

SEQ ID NO: 273 - protein, 19 aa, synthetic construct
ENSESIERAIKSTKEAIKS

SEQ ID NO: 274 - protein, 19 aa, synthetic construct
ENSERIKRALEKLTKATKS

SEQ ID NO: 275 - protein, 19 aa, synthetic construct
ENSETIEKKLKTIESRLKS

SEQ ID NO: 276 - protein, 19 aa, synthetic construct
ENSEKIKQAIEYMLKVAKS

SEQ ID NO: 277 - protein, 19 aa, synthetic construct
ENSETTKKAIELLKKLYKS

SEQ ID NO: 278 - protein, 19 aa, synthetic construct
ENSEDLKKTAAEAKKHIKS

SEQ ID NO: 279 - protein, 20 aa, synthetic construct
ENSETIKKHIEIAIKFIKEV

SEQ ID NO: 280 - protein, 20 aa, synthetic construct
ENSAKLTKATKYALTVIKQS

SEQ ID NO: 281 - protein, 20 aa, synthetic construct
ENSEEIEKAIKILKKILKES

SEQ ID NO: 282 - protein, 20 aa, synthetic construct
ENSEELKKAASKAKEEIKRS

SEQ ID NO: 283 - protein, 20 aa, synthetic construct
ENSERIKKAIKTAIEAMQKS

SEQ ID NO: 284 - protein, 20 aa, synthetic construct
ENSEKIEKILKELEKEKQSR

SEQ ID NO: 285 - protein, 20 aa, synthetic construct
ENSEEIKTIISILKELEKRS

SEQ ID NO: 286 - protein, 20 aa, synthetic construct
ENSETLKKQASKAEELEKRS

SEQ ID NO: 287 - protein, 20 aa, synthetic construct
ENSSRLKAELKKLKEILKKS

SEQ ID NO: 288 - skipped

SEQ ID NO: 289 - protein, 20 aa, synthetic construct
ENSEYIEKAIKAAQETIKKL

SEQ ID NO: 290 - protein, 20 aa, synthetic construct
ENSERIEKILKELEKEKQSR

SEQ ID NO: 291 - protein, 20 aa, synthetic construct
ENSREIIRAINIVRKIASEK

SEQ ID NO: 292 - protein, 20 aa, synthetic construct
ENSEAIERAIKDMLTAKKQS

SEQ ID NO: 293 - protein, 20 aa, synthetic construct
ENSEEILRAIKTARTESKKT

SEQ ID NO: 294 - protein, 21 aa, synthetic construct
ENSEKIKKAIEKAESIIQSIS

SEQ ID NO: 295 - protein, 21 aa, synthetic construct
ENSEETKQAIKLVKKDYKEKS

SEQ ID NO: 296 - protein, 21 aa, synthetic construct
ENSEEIDKAIKILKKILKELS

SEQ ID NO: 297 - protein, 21 aa, synthetic construct
ENSEKTKKAIKITEEIYKKLS

SEQ ID NO: 298 - protein, 21 aa, synthetic construct
ENSAKAEHAIKFALSEEKSRS

SEQ ID NO: 299 - protein, 21 aa, synthetic construct
ENSERIKKAIKTANEHLSKVN

SEQ ID NO: 300 - protein, 21 aa, synthetic construct
ENSEIIKQEIKKTQTFIKKVS

SEQ ID NO: 301 - protein, 21 aa, synthetic construct
ENSETIKREIKKTREMTKKLL

SEQ ID NO: 302 - protein, 21 aa, synthetic construct
ENSDKASKAIEYAERDAKSKS

SEQ ID NO: 303 - protein, 22 aa, synthetic construct
ENSEIWETNTERSEKKVKSIQS

SEQ ID NO: 304 - protein, 22 aa, synthetic construct
ENSEIWETNTERSIKAVLSIQS

SEQ ID NO: 305 - protein, 22 aa, synthetic construct
ENSEKIERAIKWIEDLLKKEKS

SEQ ID NO: 306 - protein, 22 aa, synthetic construct
ENSEEIKKAIKEARKAIEKLKS

SEQ ID NO: 307 - protein, 22 aa, synthetic construct
ENSEEIDKAIKEARKAIEKLKS

SEQ ID NO: 308 - protein, 22 aa, synthetic construct
ENSAKIETTKKITEELLDRAIK

SEQ ID NO: 309 - protein, 22 aa, synthetic construct
ENSEKISQAIDKTTKIILSIES

SEQ ID NO: 310 - protein, 22 aa, synthetic construct
ENSERIKQAIKKVEETLKRLKS

SEQ ID NO: 311 - protein, 22 aa, synthetic construct
ENSERLEKALQTLTKAMKKTLS

SEQ ID NO: 312 - protein, 23 aa, synthetic construct
ENSSEIKKVITETRKITKKIKSS

SEQ ID NO: 313 - protein, 23 aa, synthetic construct
ENSAKLKETTERTEKIEKKIKDS

SEQ ID NO: 314 - protein, 23 aa, synthetic construct
ENSDKLTRTAQKAKTLIEETKKS

SEQ ID NO: 315 - protein, 23 aa, synthetic construct
ENSEEIKKAIKILKKILKELSSS

SEQ ID NO: 316 - protein, 23 aa, synthetic construct
ENSDKLTRIAQKALTLIEETKKS

SEQ ID NO: 317 - protein, 23 aa, synthetic construct
ENSIRWEANAKKAETEIKKLSES

SEQ ID NO: 318 - protein, 23 aa, synthetic construct
ENSDELARAATLAKQLITKIKKS

SEQ ID NO: 319 - protein, 24 aa, synthetic construct
ENSSKIETAIKKLIEKERKTRAKK

SEQ ID NO: 320 - protein, 24 aa, synthetic construct
ENSERIKKAIEIMLSWKKALEKNS

SEQ ID NO: 321 - protein, 24 aa, synthetic construct
ENSERIKKTAKIAQKLYKTLKSQS

SEQ ID NO: 322 - protein, 24 aa, synthetic construct
ENSERIDKTAKIAQKLYKTLKSQS

SEQ ID NO: 323 - protein, 24 aa, synthetic construct
ENSEKITKAIKIAKELKKLIESML

SEQ ID NO: 324 - protein, 24 aa, synthetic construct
ENSEKITKAIKIAKELLKKIESML

SEQ ID NO: 325 - protein, 24 aa, synthetic construct
ENSEELAQTARLAKAYLKELKSRS

SEQ ID NO: 326 - protein, 24 aa, synthetic construct
ENSEKLKKAIEQMLTVKKITEKWS

SEQ ID NO: 327 - protein, 539 aa, synthetic construct
MPTSILLIITTMIMASFCQIDITKLQHVGVLVNSPKGMKISQNFETRYLILSLIPKIEDSNSCGDQQIKQYKRLLDRLIIPLYDGLRLQKDVIVSNQESNENTDPRTKRFFGGVIGTIALGVATSAQITAAVALVEAKQARSDIEKLKEAIRDTNKAVQSVQSSIGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYRVDSISYNIQNREWYIPLPSHIMTKGAFLGGADVKECIEAFSSYICPSDPGFVLNHEMESCLSGNISQCPRTVVKSDIVPRYAFVNGGVVANCITTTCTCNGIGNRINQPPDQGVKIITHKECNTIGINGMLFNTNKEGTLAFYTPNDITLNNSVALDPIDISIELNKAKSDLEESKEWIRRSNQKLDSIGNWHQSSTTIIIVLIMIIILFIINVTIIIIAVKYYRIQKRNRVDQNDKPYVLTNK

SEQ ID NO: 328 - protein, 28 aa, synthetic construct
ISIELNKLAKEVKTILKELSKKLSSLES

SEQ ID NO: 329 - protein, 26 aa, synthetic construct
ISIEMNRLKKKLDQLWKILKEDKDKS

SEQ ID NO: 330 - protein, 27 aa, synthetic construct
ISIELNKVKSKTETMAEKMRSKETATS

SEQ ID NO: 331 - protein, 27 aa, synthetic construct
ISIELNKVKSKTETYIKETRSKETATS

SEQ ID NO: 332 - protein, 26 aa, synthetic construct
ISIEMNRLKSKLDKLLKELKEDKDKS

SEQ ID NO: 333 - protein, 27 aa, synthetic construct
ISIELNKVKKETKTFIKEVRSKETATS

SEQ ID NO: 334 - protein, 32 aa, synthetic construct
ISIEVNKTQKKLKEIWKKLKKELTKERNTLKS

SEQ ID NO: 335 - protein, 24 aa, synthetic construct
ISIEVNKLKSELKTWIKQEANEKA

SEQ ID NO: 336 - protein, 25 aa, synthetic construct
ISIELNKVKSKTETYIKEVRSKETA

SEQ ID NO: 337 - protein, 28 aa, synthetic construct
ISIELNKLAKEVKTILKKLSKKLSSLES

SEQ ID NO: 338 - protein, 25 aa, synthetic construct
ISIELNKVKEDIEKLEERVHAIEKK

SEQ ID NO: 339 - protein, 23 aa, synthetic construct
ISIELNKVKERVKSLEKQLKTLL

SEQ ID NO: 340 - protein, 29 aa, synthetic construct
ISIELNKVKKKVSELEKRVDHIEHRLKQI

SEQ ID NO: 341 - protein, 27 aa, synthetic construct
ISIELNKVKDKVEKDTKKIKEIEHELA

SEQ ID NO: 342 - protein, 29 aa, synthetic construct
ISIELNKVKKELEELLQKVKDLEEKVETL

SEQ ID NO: 343 - protein, 31 aa, synthetic construct
ISIELNKVKKMVESLESKVTKLEKTVKELLT

SEQ ID NO: 344 - protein, 25 aa, synthetic construct
ISIELNKVKSELDKLKKKVEHIENS

SEQ ID NO: 345 - protein, 27 aa, synthetic construct
ISIELNKVKKDVEKLKKRISHIEKLLS

SEQ ID NO: 346 - protein, 27 aa, synthetic construct
ISIELNKVKKEVRKLEHEIHEIKKRLA

SEQ ID NO: 347 - protein, 25 aa, synthetic construct
ISIELNKVKNRVEKLEETLTRLINA

SEQ ID NO: 348 - protein, 31 aa, synthetic construct
ISIELNKVKDDLESVNKRVSEIEHELHEIKA

SEQ ID NO: 349 - protein, 33 aa, synthetic construct
ISIELNKVKEEVKELTEEIHELREEVEALKEEL

SEQ ID NO: 350 - protein, 29 aa, synthetic construct
ISIELNKVKQQVEKLIERLHRLENKLAEA

SEQ ID NO: 351 - protein, 27 aa, synthetic construct
ISIELNKVKTELHKLKERVRDIEKKLA

SEQ ID NO: 352 - protein, 29 aa, synthetic construct
ISIELNKVKKEVEELRKRLKKLEEKLTSV

SEQ ID NO: 353 - protein, 31 aa, synthetic construct
ISIELNKVKKKVSELEKQVTEIEKILTEIRA

SEQ ID NO: 354 - protein, 25 aa, synthetic construct
ISIELNKVKERLHKLEESVKQLKKA

SEQ ID NO: 355 - protein, 23 aa, synthetic construct
ISIELNKVKSDVENLKEKINKII

SEQ ID NO: 356 - protein, 29 aa, synthetic construct
ISIELNKVKDDVRTIKKELEELKQLVKNL

SEQ ID NO: 357 - protein, 35 aa, synthetic construct
ISIELNKVKTRVEEIERKISSLEKEVEDIRRSLQQ

SEQ ID NO: 358 - protein, 35 aa, synthetic construct
ISIELNKVKNKLEKVESQVHRLENRIEKIERLLKS

SEQ ID NO: 359 - protein, 33 aa, synthetic construct
ISIELNKVKRDVEQLRQELNSLSKRVHKIEEAL

SEQ ID NO: 360 - protein, 25 aa, synthetic construct
ISIELNKVKSAVTHLTKEVTKLKEL

SEQ ID NO: 361 - protein, 27 aa, synthetic construct
ISIELNKVKKDLNDAKKRISHIEKVLN

SEQ ID NO: 362 - protein, 33 aa, synthetic construct
ISIELNKVKADLTTLESKQSEIERRVAKIEHAL

SEQ ID NO: 363 - protein, 33 aa, synthetic construct
ISIELNKVKEEVEKLERETKKLSHEIKKIKETL

SEQ ID NO: 364 - protein, 35 aa, synthetic construct
ISIELNKVKSEVSELKTKVQTLETRIKKIEHELKL

SEQ ID NO: 365 - protein, 33 aa, synthetic construct
ISIELNKVKKKVEKIEKEIEKLKRELETVKREI

SEQ ID NO: 366 - protein, 31 aa, synthetic construct
ISIELNKVKKKVESLERKVSKLENEIKTIID

SEQ ID NO: 367 - protein, 23 aa, synthetic construct
ISIELNKVKKDVTYLKTEVAQLQ

SEQ ID NO: 368 - protein, 33 aa, synthetic construct
ISIELNKVKKEVKELKERLDHVEKRLKEVEEKL

SEQ ID NO: 369 - protein, 25 aa, synthetic construct
ISIELNKVKEDVASLKKEVEKIIKA

SEQ ID NO: 370 - protein, 23 aa, synthetic construct
ISIELNKVKNSLDKVEKKVTSLI

SEQ ID NO: 371 - protein, 27 aa, synthetic construct
ISIELNKVKERVKENEKIITKIQKTLD

SEQ ID NO: 372 - protein, 35 aa, synthetic construct
ISIELNKVKTEVKEITKKVRELEERLRKVEEVVKS

SEQ ID NO: 373 - protein, 29 aa, synthetic construct
ISIELNKVKSDVRDLEERLHKLETRLEEI

SEQ ID NO: 374 - protein, 25 aa, synthetic construct
ISIELNKVKSEVKKLKERLEELEAR

SEQ ID NO: 375 - protein, 27 aa, synthetic construct
ISIELNKVKEKVDKIQENIDAIKTILD

SEQ ID NO: 376 - protein, 31 aa, synthetic construct
ISIELNKVKNEVSELEKRTTKIESTIKTLIE

SEQ ID NO: 377 - protein, 25 aa, synthetic construct
ISIELNKVKKDLKELSEKVHELLNS

SEQ ID NO: 378 - protein, 29 aa, synthetic construct
ISIELNKVKKRLEELEEKLDRLEHIVHLL

SEQ ID NO: 379 - protein, 23 aa, synthetic construct
ISIELNKVKENVEEIEHKVKEIE

SEQ ID NO: 380 - protein, 35 aa, synthetic construct
ISIELNKVKKEVNELNKRIRSLEQRVEKLERALKK

SEQ ID NO: 381 - protein, 31 aa, synthetic construct
ISIELNKVKKDLKKTKENLKEVEEKVKELLS

SEQ ID NO: 382 - protein, 529 aa, synthetic construct
MGTIIQFLVVSCLLAGAGSLDPAALMQIGVIPTNVRQLMYYTEASSAFIVVKLMPTIDSPISGCNITSISSYNATVTKLLQPIGENLETIRNQLIPTRRRRRFAGVVIGLAALGVATAAQVTAAVALVKANENAAAILNLKNAIQKTNAAVADVVQATQSLGTAVQAVQDHINSVVSPAITAANCKAQDAIIGSILNLYLTELTTIFHNQITNPALSPITIQALRILLGSTLPTVVEKSFNTQISAAELLSSGLLTGQIVGLDLTYMQMVIKIELPTLTVQPATQIIDLATISAFINNQEVMAQLPTRVMVTGSLIQAYPASQCTITPNTVYCRYNDAQVLSDDTMACLQGNLTRCTFSPVVGSFLTRFVLFDGIVYANCRSMLCKCMQPAAVILQPSSSPVTVIDMYKCVSLQLDNLRFTITQLANVTYNSTIKLESSQILSIDPLDISQNLAAVNKSLSDALQHLAQSDTYLSAITSATTTSVLSIIAICLGSLGLILIILLSVVVWKLLTIVVANRNRMENFVYHK

SEQ ID NO: 383 - protein, 16 aa, synthetic construct
SLSDLKKKVDEATKTT

SEQ ID NO: 384 - protein, 26 aa, synthetic construct
SLSDLIKAITKKEEKSTRKERSERKS

SEQ ID NO: 385 - protein, 15 aa, synthetic construct
SLSDTIKKLDKLVKS

SEQ ID NO: 386 - protein, 11 aa, synthetic construct
SLSDLIKEVKS

SEQ ID NO: 387 - protein, 18 aa, synthetic construct
SLSDTQKLVTEILEKLTK

SEQ ID NO: 388 - protein, 24 aa, synthetic construct
SLSDVIQIMLETLETATKQKKKDS

SEQ ID NO: 389 - protein, 13 aa, synthetic construct
SLSDLAKKFKEAS

SEQ ID NO: 390 - protein, 15 aa, synthetic construct
SLSDLKKKLDELEKR

SEQ ID NO: 391 - protein, 20 aa, synthetic construct
SLSDTIKKVDKSTKSTEKKS

SEQ ID NO: 392 - protein, 22 aa, synthetic construct
SLSDVAKKLEEKIRTDIKREQS

SEQ ID NO: 393 - protein, 24 aa, synthetic construct
SLSDTITIMKKIEEKLKADKKKSS

SEQ ID NO: 394 - protein, 18 aa, synthetic construct
SLSDVIKWVREVVSKWIS

SEQ ID NO: 395 - protein, 16 aa, synthetic construct
SLSDLKKKVDTLEKQS

SEQ ID NO: 396 - protein, 13 aa, synthetic construct
SLSDLWKIMEKLS

SEQ ID NO: 397 - protein, 12 aa, synthetic construct
SLSDLKKKVDSK

SEQ ID NO: 398 - protein, 24 aa, synthetic construct
SLSDLAKKLDKTIEKASKDDSKKS

SEQ ID NO: 399 - protein, 21 aa, synthetic construct
SLSDVAKRAESTIRDLKETKK

SEQ ID NO: 400 - protein, 14 aa, synthetic construct
SLSDLATKVEKALS

SEQ ID NO: 401 - protein, 15 aa, synthetic construct
SLSDLIKKTDALEKS

SEQ ID NO: 402 - protein, 16 aa, synthetic construct
SLSDLIKKVITLEKKS

SEQ ID NO: 403 - protein, 26 aa, synthetic construct
SLSDLKKKTEEIATDLEKKWRKMSKS

SEQ ID NO: 404 - protein, 20 aa, synthetic construct
SLSDLKKKLDSILTEQKRRS

SEQ ID NO: 405 - protein, 16 aa, synthetic construct
SLSDVIKKLDEALSRI

SEQ ID NO: 406 - protein, 12 aa, synthetic construct
SLSDTIKEMKEK

SEQ ID NO: 407 - protein, 22 aa, synthetic construct
SLSDLAEKCKKLKKKLEEDLKS

SEQ ID NO: 408 - protein, 14 aa, synthetic construct
SLSDVIKEIRKLKS

SEQ ID NO: 409 - protein, 14 aa, synthetic construct
SLSDLAKIVKSLIS

SEQ ID NO: 410 - protein, 22 aa, synthetic construct
SLSDLKKKLEEILASIEKKEKS

SEQ ID NO: 411 - protein, 24 aa, synthetic construct
SLSDTIKELKSHLTTLKIEKSKKS

SEQ ID NO: 412 - protein, 13 aa, synthetic construct
SLSDLKEKLDRYI

SEQ ID NO: 413 - protein, 15 aa, synthetic construct
SLSDLKTKIEQILKS

SEQ ID NO: 414 - protein, 18 aa, synthetic construct
SLSDVIKKLDKIVKKLQS

SEQ ID NO: 415 - protein, 15 aa, synthetic construct
SLSDLASKVETETRK

SEQ ID NO: 416 - protein, 26 aa, synthetic construct
SLSDLAKRTKTWYDILAKILASNQKS

SEQ ID NO: 417 - protein, 21 aa, synthetic construct
SLSDTAKIALTVEKILTTRDK

SEQ ID NO: 418 - protein, 13 aa, synthetic construct
SLSDTQKLLKELI

SEQ ID NO: 419 - protein, 18 aa, synthetic construct
SLSDVIKKVETIASKLKS

SEQ ID NO: 420 - protein, 14 aa, synthetic construct
SLSDAIKKIDKLES

SEQ ID NO: 421 - protein, 20 aa, synthetic construct
SLSDTISILEEFLRRYKQKE

SEQ ID NO: 422 - protein, 18 aa, synthetic construct
SLSDTQKQLETLAKKIKS

SEQ ID NO: 423 - protein, 20 aa, synthetic construct
SLSDLAKRVKKYWEEVKSRS

SEQ ID NO: 424 - protein, 20 aa, synthetic construct
SLSDLAKELKKLKEHILRYQ

SEQ ID NO: 425 - protein, 20 aa, synthetic construct
SLSDTIKLVIKAILTAIKEK

SEQ ID NO: 426 - protein, 14 aa, synthetic construct
SLSDTIKKVDKLTS

SEQ ID NO: 427 - protein, 26 aa, synthetic construct
SLSDTIKKLEKLERELRSRWDSERKS

SEQ ID NO: 428 - protein, 30 aa, synthetic construct
SLSDTIKTTEKALKIILKRIKKALAEQKSS

SEQ ID NO: 429 - protein, 11 aa, synthetic construct
SLSDLIKKFNS

SEQ ID NO: 430 - protein, 12 aa, synthetic construct
SLSDLKKTLEKR

SEQ ID NO: 431 - protein, 14 aa, synthetic construct
SLSDLESELKSRLS

SEQ ID NO: 432 - protein, 13 aa, synthetic construct
SLSDVIKDLKKTK

SEQ ID NO: 433 - protein, 11 aa, synthetic construct
SLSDLAKKLDS

SEQ ID NO: 434 - protein, 15 aa, synthetic construct
SLSDVIKIIESQTRS

SEQ ID NO: 435 - protein, 16 aa, synthetic construct
SLSDLKKETEKLKKKV

SEQ ID NO: 436 - protein, 22 aa, synthetic construct
SLSDAIKRVLSWYKKKADEESS

SEQ ID NO: 437 - protein, 18 aa, synthetic construct
SLSDVKKKVDKAITEIKS

SEQ ID NO: 438 - protein, 12 aa, synthetic construct
SLSDLAKEVKKK

SEQ ID NO: 439 - protein, 13 aa, synthetic construct
SLSDLKKKLEKIL

SEQ ID NO: 440 - protein, 15 aa, synthetic construct
SLSDLASDVSSMKAT

SEQ ID NO: 441 - protein, 15 aa, synthetic construct
SLSDTIKKLEELTTK

SEQ ID NO: 442 - protein, 20 aa, synthetic construct
SLSDLKKTTEKVIRTLKTKE

SEQ ID NO: 443 - protein, 20 aa, synthetic construct
SLSDLKKEHEELLKEIKKQK

SEQ ID NO: 444 - protein, 20 aa, synthetic construct
SLSDLATKTKQLEEKLEKEK

SEQ ID NO: 445 - protein, 20 aa, synthetic construct
SLSDLKKRTIKWYEETLKRT

SEQ ID NO: 446 - protein, 18 aa, synthetic construct
SLSDLAKKTKEAIDRIRS

SEQ ID NO: 447 - protein, 14 aa, synthetic construct
SLSDLQTDIKRLKS

SEQ ID NO: 448 - protein, 18 aa, synthetic construct
SLSDLAKKTKELEKKIKS

SEQ ID NO: 449 - protein, 26 aa, synthetic construct
SLSDLAKKAKKFTEKLLSEIKKTKSD

SEQ ID NO: 450 - protein, 10 aa, synthetic construct
SLSDLAKYVS

SEQ ID NO: 451 - protein, 30 aa, synthetic construct
SLSDTQKKTKETATKLEQKTEKTLKYTKKK

SEQ ID NO: 452 - protein, 12 aa, synthetic construct
SLSDLKKKVDKK

SEQ ID NO: 453 - protein, 22 aa, synthetic construct
SLSDLARKTKEYWEKEERSKKS

SEQ ID NO: 454 - protein, 20 aa, synthetic construct
SLSDLKKRLEDYIKTQKAKS

SEQ ID NO: 455 - protein, 16 aa, synthetic construct
SLSDLKKKLDELTKKS

SEQ ID NO: 456 - protein, 10 aa, synthetic construct
SLSDLIKEVK

SEQ ID NO: 457 - protein, 26 aa, synthetic construct
SLSDVIKILKEIKEMLDKLLEKSKKS

SEQ ID NO: 458 - protein, 18 aa, synthetic construct
SLSDLAKQTKKLEDELRS

SEQ ID NO: 459 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 460 - protein, 29 aa, synthetic construct
LQPELETAIKITLEIVLKILKEWEKRKSS

SEQ ID NO: 461 - protein, 15 aa, synthetic construct
LQPELDSAASYAIKV

SEQ ID NO: 462 - protein, 23 aa, synthetic construct
LQPELETAASIAEKIARKLLKES

SEQ ID NO: 463 - protein, 23 aa, synthetic construct
LQPELESAIKKTLKIISKRNKDS

SEQ ID NO: 464 - protein, 21 aa, synthetic construct
LQPELEKAIKKATEIARKLIS

SEQ ID NO: 465 - protein, 23 aa, synthetic construct
LQPELESAADKTMKKYKTEAKRS

SEQ ID NO: 466 - protein, 25 aa, synthetic construct
LQPELETALRIAIEITLQLLKKMAS

SEQ ID NO: 467 - protein, 21 aa, synthetic construct
LQPELEKAIKITLKIIDIKLS

SEQ ID NO: 468 - protein, 19 aa, synthetic construct
LQPELEKAAKKALEIASRS

SEQ ID NO: 469 - protein, 23 aa, synthetic construct
LQPELEKAIKKTLKIIWTELSIS

SEQ ID NO: 470 - protein, 17 aa, synthetic construct
LQPELESAMKTAMKIIS

SEQ ID NO: 471 - protein, 19 aa, synthetic construct
LQPELKKAMETAIKRINKA

SEQ ID NO: 472 - protein, 25 aa, synthetic construct
LQPELEKAAKKTLKIAKEESTKDKS

SEQ ID NO: 473 - protein, 23 aa, synthetic construct
LQPELEKAIKKTLKIIRTELSIS

SEQ ID NO: 474 - protein, 21 aa, synthetic construct
LQPELESAIKKALTIIKQIWS

SEQ ID NO: 475 - protein, 27 aa, synthetic construct
LQPELDSAASRALKIAIELLRATESKK

SEQ ID NO: 476 - protein, 25 aa, synthetic construct
LQPELEKAASKAIKISLKILKEILS

SEQ ID NO: 477 - protein, 15 aa, synthetic construct
LQPELEKAIKEALKR

SEQ ID NO: 478 - protein, 21 aa, synthetic construct
LQPELETAIKIALEIARKEIS

SEQ ID NO: 479 - protein, 17 aa, synthetic construct
LQPELEKAAKTALKIAS

SEQ ID NO: 480 - protein, 27 aa, synthetic construct
LQPELEKAAEEAVRRAIKLYKENLKKS

SEQ ID NO: 481 - protein, 21 aa, synthetic construct
LQPELQTLKEESTHLTKTLLS

SEQ ID NO: 482 - protein, 25 aa, synthetic construct
LQPELTKLKEEVLEEVETMIRETAA

SEQ ID NO: 483 - protein, 17 aa, synthetic construct
LQPELENLKNTVESIIN

SEQ ID NO: 484 - protein, 19 aa, synthetic construct
LQPELSKTKAETLETVREL

SEQ ID NO: 485 - protein, 23 aa, synthetic construct
LQPELEKTQSTTLTAAKTLIKST

SEQ ID NO: 486 - protein, 19 aa, synthetic construct
LQPELETTKKETLTEVTEA

SEQ ID NO: 487 - protein, 17 aa, synthetic construct
LQPELERIRTEVTQASA

SEQ ID NO: 488 - protein, 21 aa, synthetic construct
LQPELESTKAVTETEIKAEIN

SEQ ID NO: 489 - protein, 23 aa, synthetic construct
LQPELNTTKTETISSIKKEIETM

SEQ ID NO: 490 - protein, 19 aa, synthetic construct
LQPELEATHTRTLTTVTAA

SEQ ID NO: 491 - protein, 23 aa, synthetic construct
LQPELDTTKKETLTEAQETLERA

SEQ ID NO: 492 - protein, 23 aa, synthetic construct
LQPELDKVKDETVTIMTKYIQET

SEQ ID NO: 493 - protein, 21 aa, synthetic construct
LQPELDATSSRAIERVTTLLE

SEQ ID NO: 494 - protein, 23 aa, synthetic construct
LQPELETTRTKTITEVNTTISTT

SEQ ID NO: 495 - protein, 27 aa, synthetic construct
LQPELEAVKTETLTAATTAINSALAKQ

SEQ ID NO: 496 - protein, 21 aa, synthetic construct
LQPELKETQEKTITEVIKILN

SEQ ID NO: 497 - protein, 19 aa, synthetic construct
LQPELTNTENNVLTRVKQS

SEQ ID NO: 498 - protein, 17 aa, synthetic construct
LQPELNALETRVLTAIN

SEQ ID NO: 499 - protein, 546 aa, synthetic construct
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 500 - protein, 24 aa, synthetic construct
ISSINEDMERTKKWITKLIAKWKS

SEQ ID NO: 501 - protein, 22 aa, synthetic construct
ISSINEALKSLATDVKKLKSKI

SEQ ID NO: 502 - protein, 34 aa, synthetic construct
ISSANLEIEKTKRKMTSIAKEVKTRIAKEEKSKS

SEQ ID NO: 503 - protein, 20 aa, synthetic construct
ISSTNLTVEKIWRYLMAVLS

SEQ ID NO: 504 - protein, 28 aa, synthetic construct
ISSTNKRTATIEKIVRSLLKEIKSERTR

SEQ ID NO: 505 - protein, 26 aa, synthetic construct
ISSINETVTRLKKIVEKLIRELQKIK

SEQ ID NO: 506 - protein, 27 aa, synthetic construct
ISSTNTIVSKTLKMLLEFITREERSKR

SEQ ID NO: 507 - protein, 24 aa, synthetic construct
ISSTNSLTEKILQWIKKFETKVKS

SEQ ID NO: 508 - protein, 32 aa, synthetic construct
ISSTNLIVTETIKELKSTDKKLKKYIKTVQSS

SEQ ID NO: 509 - protein, 22 aa, synthetic construct
ISSANKIMAEIIKTIKSLLKKS

SEQ ID NO: 510 - protein, 34 aa, synthetic construct
ISSANLEIEKTKRIMTSIALYVWTLIAKELKSKS

SEQ ID NO: 511 - protein, 36 aa, synthetic construct
ISSINEEIKKVKKTAAEAITTQTRIWQKLKKSKSKS

SEQ ID NO: 512 - protein, 34 aa, synthetic construct
ISSLNEKIDKLEKKMSTIAKKLSKIEASKRKSSS

SEQ ID NO: 513 - protein, 24 aa, synthetic construct
ISSTNIRVTKTEKKVEDLLKKLTS

SEQ ID NO: 514 - protein, 19 aa, synthetic construct
ISSINELVTRLAKILKKLI

SEQ ID NO: 515 - protein, 19 aa, synthetic construct
ISSINEQVKKIEEILRSMS

SEQ ID NO: 516 - protein, 34 aa, synthetic construct
ISSANLKIETLARIVSTWYKQQAKKTATEEKRKS

SEQ ID NO: 517 - protein, 24 aa, synthetic construct
ISSMNTRIDQIEKWLRDKEKKEQS

SEQ ID NO: 518 - protein, 20 aa, synthetic construct
ISSINEETKKVKKIALDIAS

SEQ ID NO: 519 - protein, 28 aa, synthetic construct
ISSINEKIDSLKKEVKKYIEKAEKDKKS

SEQ ID NO: 520 - protein, 22 aa, synthetic construct
ISSLNDLVRKALKWIKEVKKKS

SEQ ID NO: 521 - protein, 28 aa, synthetic construct
ISSLNEKIIKILQKLLTWITKTKQEKKS

SEQ ID NO: 522 - protein, 29 aa, synthetic construct
ISSLRQKISSLEKALKKAEKDLEEVRRQL

SEQ ID NO: 523 - protein, 15 aa, synthetic construct
ISSLTTEVKQLQTSL

SEQ ID NO: 524 - protein, 21 aa, synthetic construct
ISSLTNSITSLSERIHKLENL

SEQ ID NO: 525 - protein, 27 aa, synthetic construct
ISSLTDRLDNLEERVKRLEEEVKKLKE

SEQ ID NO: 526 - protein, 27 aa, synthetic construct
ISSITEQLKEAQERVDKIEKLLEKILR

SEQ ID NO: 527 - protein, 15 aa, synthetic construct
ISSLTSAITAIQETL

SEQ ID NO: 528 - protein, 19 aa, synthetic construct
ISSLRKEIKELRTVVKRLL

SEQ ID NO: 529 - protein, 15 aa, synthetic construct
ISSLTRSIKDVKQAL

SEQ ID NO: 530 - protein, 15 aa, synthetic construct
ISSITSEITELKKTL

SEQ ID NO: 531 - protein, 25 aa, synthetic construct
ISSLQKNVESLAKEVKKLEQKLNSL

SEQ ID NO: 532 - protein, 29 aa, synthetic construct
ISSLRQEIKNLQDEVTKVTEELKKLVEQL

SEQ ID NO: 533 - protein, 17 aa, synthetic construct
ISSVKTNVRKLSEILAS

SEQ ID NO: 534 - protein, 25 aa, synthetic construct
ISSLNKKIEEIEKRLSELESTIKKL

SEQ ID NO: 535 - protein, 27 aa, synthetic construct
ISSLQSLAESLADKVTALETRIKSIEA

SEQ ID NO: 536 - protein, 17 aa, synthetic construct
ISSLSKRVKSVETRLRT

SEQ ID NO: 537 - protein, 23 aa, synthetic construct
ISSITTDIKQNTERIDKIEKTLK

SEQ ID NO: 538 - protein, 23 aa, synthetic construct
ISSLTRAVRKLEKRLTHVEEVLK

SEQ ID NO: 539 - protein, 15 aa, synthetic construct
ISSITKEIKSLDTRL

SEQ ID NO: 540 - protein, 27 aa, synthetic construct
ISSITKKVDSLLTEVHAIRHEIDQLRS

SEQ ID NO: 541 - protein, 23 aa, synthetic construct
ISSIREQISTITTEIKKIKEILL

SEQ ID NO: 542 - protein, 29 aa, synthetic construct
ISSLTDEISKLSNRVQRLERRLQEIERRL

SEQ ID NO: 543 - protein, 23 aa, synthetic construct
ISSLTERVERLETLVREVQKQLE

SEQ ID NO: 544 - protein, 17 aa, synthetic construct
ISSLTEKIESIEKDIAT

SEQ ID NO: 545 - protein, 29 aa, synthetic construct
ISSLAKRLDELSSQLADLSARVEALQSTL

SEQ ID NO: 546 - protein, 27 aa, synthetic construct
ISSLTNHIKDLAKRVSDIESLVQKLLS

SEQ ID NO: 547 - protein, 29 aa, synthetic construct
ISSITSSISRNTDKIKELQQEIEKLQSSL

SEQ ID NO: 548 - protein, 19 aa, synthetic construct
ISSLTRDVDKLNSQIQALI

SEQ ID NO: 549 - protein, 27 aa, synthetic construct
ISSLTAVASENTARIEALERRIHELEL

SEQ ID NO: 550 - protein, 25 aa, synthetic construct
ISSLKEEVTNLKKRLSEVEKVIKTL

SEQ ID NO: 551 - protein, 21 aa, synthetic construct
ISSITEQLQRLSERVEEIERR

SEQ ID NO: 552 - protein, 23 aa, synthetic construct
ISSLNTQVKKLKDRIKKIEERLN

SEQ ID NO: 553 - protein, 29 aa, synthetic construct
ISSLQSEVSNLRTDLNDLKKLVKKLIELL

SEQ ID NO: 554 - protein, 27 aa, synthetic construct
ISSITKDIQKNTERINKIEKTIKSLIS

SEQ ID NO: 555 - protein, 23 aa, synthetic construct
NSQTTEEQIKTLTERVESIEKEG

SEQ ID NO: 556 - protein, 27 aa, synthetic construct
NSQNIEDRVEDNDDKVAELKEELEAIK

SEQ ID NO: 557 - protein, 29 aa, synthetic construct
NSQNVEDRLEELESRIKKIEEEIEEIKKD

SEQ ID NO: 558 - protein, 29 aa, synthetic construct
NSQNIEEDLESLKERIHRLESEVQNLLER

SEQ ID NO: 559 - protein, 19 aa, synthetic construct
NSQKIQDAVEELQTLMQKL

SEQ ID NO: 560 - protein, 25 aa, synthetic construct
NSQRTEKRINDLESRVARIEEVLSL

SEQ ID NO: 561 - protein, 27 aa, synthetic construct
NSQETEDTLESLSQEVEKLRETVEKLT

SEQ ID NO: 562 - protein, 25 aa, synthetic construct
NSQNILDRINENEQRVSVLERTLAQ

SEQ ID NO: 563 - protein, 33 aa, synthetic construct
NSQSIEDSLSTLNTKINKLKKEVESLKREVEEL

SEQ ID NO: 564 - protein, 31 aa, synthetic construct
NSQEIDKKLEYLEERVHDLEERLESLVQQLQ

SEQ ID NO: 565 - protein, 27 aa, synthetic construct
NSQNVEDRLEANEKAISHIEQLIDQLI

SEQ ID NO: 566 - protein, 19 aa, synthetic construct
LXXTIXXLLXIXXXLXXXL

SEQ ID NO: 567 - protein, 24 aa, synthetic construct
LVXTXKXLXDLIXXLXXLLXKLXX

SEQ ID NO: 568 - protein, 23 aa, synthetic construct
LNKVKKXVXXLXXXVXXLEKXLX

SEQ ID NO: 569 - protein, 13 aa, synthetic construct
EKIXXAIKKAXKL

SEQ ID NO: 570 - protein, 15 aa, synthetic construct
EXIXKAIKXLXXXXX

SEQ ID NO: 571 - skipped

SEQ ID NO: 572 - protein, 17 aa, synthetic construct
XXLKKAAXIXKKXLKXX

SEQ ID NO: 573 - protein, 21 aa, synthetic construct
LXXTIXXLLXIVIXXLILXXL

SEQ ID NO: 574 - protein, 26 aa, synthetic construct
LVATTXKXLXDLIXXLKEXLLXKLXX

SEQ ID NO: 575 - protein, 23 aa, synthetic construct
LNKVKKXVXXLXXXVXXLEKXLX

SEQ ID NO: 576 - protein, 13 aa, synthetic construct
EKIXXAIKKAXKL

SEQ ID NO: 577 - protein, 17 aa, synthetic construct
EXIXKAIKXLLXXXXXX

SEQ ID NO: 578 - protein, 25 aa, synthetic construct
XKXTLEETAXXIXVXXXXXXXXXXX

SEQ ID NO: 579 - protein, 17 aa, synthetic construct
XXLKKAAXIXKKXLKXX

SEQ ID NO: 580 - protein, 6 aa, synthetic construct
QVKRNL

SEQ ID NO: 581 - protein, 8 aa, synthetic construct
STLAQKEY

SEQ ID NO: 582 - protein, 5 aa, synthetic construct
IVNTL

SEQ ID NO: 583 - protein, 6 aa, synthetic construct
QNKRVS

SEQ ID NO: 584 - protein, 6 aa, synthetic construct
ANKEDQ

SEQ ID NO: 585 - protein, 4 aa, synthetic construct
TMVR

SEQ ID NO: 586 - protein, 8 aa, synthetic construct
TIKQMEVS

SEQ ID NO: 587 - protein, 5 aa, synthetic construct
SKNDE

SEQ ID NO: 588 - protein, 7 aa, synthetic construct
RSEKATL

SEQ ID NO: 589 - protein, 4 aa, synthetic construct
VNTL

SEQ ID NO: 590 - protein, 7 aa, synthetic construct
DTHKENR

SEQ ID NO: 591 - protein, 8 aa, synthetic construct
ANESVKTD

SEQ ID NO: 592 - protein, 5 aa, synthetic construct
IELTQ

SEQ ID NO: 593 - protein, 6 aa, synthetic construct
EIKNRQ

SEQ ID NO: 594 - protein, 5 aa, synthetic construct
ASKER

SEQ ID NO: 595 - protein, 6 aa, synthetic construct
KSQRDE

SEQ ID NO: 596 - skipped

SEQ ID NO: 597 - protein, 5 aa, synthetic construct
KQENT

SEQ ID NO: 598 - protein, 7 aa, synthetic construct
HDEKRNQ

SEQ ID NO: 599 - protein, 4 aa, synthetic construct
LRIV

SEQ ID NO: 600 - protein, 5 aa, synthetic construct
EVLKR

SEQ ID NO: 601 - protein, 5 aa, synthetic construct
AKTER

SEQ ID NO: 602 - protein, 7 aa, synthetic construct
HRSLNED

SEQ ID NO: 603 - protein, 4 aa, synthetic construct
ILVR

SEQ ID NO: 604 - protein, 4 aa, synthetic construct
EKQD

SEQ ID NO: 605 - protein, 5 aa, synthetic construct
DKSRA

SEQ ID NO: 606-609 - skipped (4 entries)

SEQ ID NO: 610 - protein, 519 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 611 - protein, 518 aa, synthetic construct
MELLILKANAITTILTAVTFCFASGQNITEEFYQSTCSAVSKGYLSALRTGWYHSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAWSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 612 - protein, 519 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 613 - protein, 519 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLCGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMCSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 614 - protein, 518 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 615 - protein, 519 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 616 - protein, 519 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 617 - protein, 519 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 618 - protein, 519 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 619 - protein, 519 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLCGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMCSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSQFSASISQVNEKINQSREIIRAINIVRKIASEK

SEQ ID NO: 620 - protein, 518 aa, synthetic construct
MELLILKANAITTILTAVTFCFASGQNITEEFYQSTCSAVSKGYLSALRTGWYHSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEILAYWQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 621 - protein, 518 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEK

SEQ ID NO: 622 - protein, 509 aa, synthetic construct
MELLILKAAITTILTAVTFCFASGQNITEEFYQSTCSAVSKGYLSALRTGWYTSCITIELSNIKENKCNGTDAVKLIKQELDKYKNAVTELQLLMQSTPATNNRARRELPRFMYTLNNAKKTVTLSKKRKRRFLGFLLGVGSAIASGVAVSKVLHLEGECNKIKSALLSTNKAVVSLSNGVSVLTSKVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEK

SEQ ID NO: 623 - protein, 507 aa, synthetic construct
MELLILKAAITTILTAVTFCFASGQNITEEFYQSTCSAVSKGYLSALRTGWYTSVCTIELSNIKENKCNGTDAVKLIKQELDKYKNAVTELQLLMQSTPATNNRARRELPRFMYTLNNAKKTVTLSKKRKRRFLGFLLGVGSAIASGVAVSBVLHLEGEVKIKSALLSTNKAWSLSNGVSVLTCBVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEK

SEQ ID NO: 624 - protein, 509 aa, synthetic construct
MELLILKAAITTILTAVTFCFASGQNITEEFYQSTCSAVSKGYLSALRTGWYTSVICIELSNIKENKCNGTDAVKLIKQELDKYKNAVTELQLLMQSTPATNNRARRELPRFMYTLNNAKKTVTLSKKRKRRFLGFLLGVGSAIASGVAVSKVLHLEGECNKIKSALLSTNKAVVSLSNGVSVLTSKVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEK

SEQ ID NO: 625 - protein, 508 aa, synthetic construct
MELLILKAAITTILTAVTFCFASGQNITEEFYQSTCSAVSKGYLSALRTGWYTSVITIELSNIKENKCNGTDAVKLIKQELDKYKNAVTELQLLMQSTPATNNRARRELPRFMYTLNNAKKTVTLSKKRKRRFLGFLLGVGSAIASGVAVSBVLHLEGEVCKIKSALLSTNKAWSLSNGVSVLTSBVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEECLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEK

SEQ ID NO: 626 - protein, 506 aa, synthetic construct
MELLILKAAITTILTAVTFCFASGQNITEEFYQSTCSAVSKGYLSALRTGWYTSVITIELSNIKENKCNGTDAKVKLIKQELDKYKNAVTELQLLMQSTPATIWRARRELPRFMYTLAKKTVTLSKKRKRRFLGFLLGVGSAIASGVAVSBVLHLEGEVKICSALLSTNKAWSLSNGVSVLTSBVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEECLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEK

SEQ ID NO: 627 - protein, 508 aa, synthetic construct
MELLILKAAITTILTAVTFCFASGQNITEEFYQSTCSAVSKGYLSALRTGWYTSVITIELSNIKENKCNGTDAVKLIKQELDKYKNAVTELQLLMQSTPATNNRARRELPRFMYTLNNAKKTVTLSKKRKRRFLGFLLGVGSAIASGVAVSKVLHLEGEVKIKSALLSTNKAVVSLSNGVSVLTSKVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKICTSKTDVSSSVITSLGAIVSCYGKTKCTASNKNRGIIKTFSNGCDYVSNKGVDTVSVGNTLYYVKQEGKSLYVKGEPIINFYDPLVCPSDEFDASISQVEKINQSREIIRAINIVRKIASEK

SEQ ID NO: 628 - protein, 519 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 629 - protein, 519 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 630 - protein, 519 aa, synthetic construct; identical to SEQ ID NO: 628

SEQ ID NO: 631 - protein, 519 aa, synthetic construct; identical to SEQ ID NO: 629

SEQ ID NO: 632 - protein, 496 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARQQQQRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 633 - protein, 489 aa, synthetic construct
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 634 - protein, 509 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKQKQQAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 635 - protein, 495 aa, synthetic construct
METPAQLLFLLLLWLPDTTGFASGQNITEEFYQSTCSAVSKGYLSALRTGWYTSVITIELSNIKKNKCNGTDAKVKLIKQELDKYKNAVTELQLLMQSTQATNNRARQQQQRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 636 - protein, 495 aa, synthetic construct; identical to SEQ ID NO: 635

SEQ ID NO: 637 - protein, 519 aa, synthetic construct; identical to SEQ ID NO: 629

SEQ ID NO: 638 - protein, 518 aa, synthetic construct
MELLILKANAITTILTAVTFCFASQNITEEFYQSTCSAVSKGYLSALRTGWYTSVITIELSNIKENKCNGTDAKVKLIKQELDKYKNAVTELQLLMQSTPATNNRARRELPRFMNYTLNNAKKINVILSKKRKRRFLGFLLGVGSAIASGVAVCKVLHLEGEVNKIKSALLSINKAVVSLSNGVSVLIFKVLDLKNYIDKQLLPILNKQSCSISNIETVIEFQQKNNRLLEITREFSVNAGVITPVSTYMLINSELLSLINDMPITNDQKKLMSNNVQIVRQQSYSIMCIIKEEVLAYVVQLPLYGVIDTPCWKLHISPLCTINTKEGSNICLTRIDRGWYCDNAGSVSFFPQAETCKVQSNRVFCDTMNSLTLPSEVNLCNVDIFNPKYDCKIMISKTDVSSSVITSLGAIVSCYGKTKCIASNKNRGIIKTFSNGCDYVSNKGVDTVSVGNTLYYVNKQEGKSLYVKGEPIINFYDPLVFPSDEFDASISQVNEKINQSREIIRAINIVRKIASEK

SEQ ID NO: 639 - protein, 15 aa, synthetic construct
NNQARGSGSGRSLGF

SEQ ID NO: 640 - protein, 15 aa, synthetic construct
NNQARGGSGGRSLGF

SEQ ID NO: 641 - protein, 15 aa, synthetic construct
NNGARGGSGGRSLGF

SEQ ID NO: 642 - protein, 15 aa, synthetic construct
NNQARGGSGGDSLGF

SEQ ID NO: 643 - protein, 17 aa, synthetic construct
NNQARGGSGSGGDSLGF

SEQ ID NO: 644 - protein, 14 aa, synthetic construct
NNQARGGSGGGDLG

SEQ ID NO: 645 - protein, 16 aa, synthetic construct
NNQARGGSGSGGDLGF

SEQ ID NO: 646 - skipped

SEQ ID NO: 647 - protein, 4 aa, synthetic construct
GSGS

SEQ ID NO: 648 - protein, 7 aa, synthetic construct
GGSGEKP

SEQ ID NO: 649 - protein, 7 aa, synthetic construct
GGSGQKP

SEQ ID NO: 650 - protein, 8 aa, synthetic construct
GGSGGSGS

SEQ ID NO: 651 - protein, 10 aa, synthetic construct
GGSGGSGEKP

SEQ ID NO: 652 - protein, 10 aa, synthetic construct
GGSGGSGQKP

SEQ ID NO: 653 - protein, 12 aa, synthetic construct
GGSGGSGGSGGS

SEQ ID NO: 654 - protein, 12 aa, synthetic construct
GSGGSGSGSGGS

SEQ ID NO: 655 - protein, 15 aa, synthetic construct
GGGGGSGGGSGGGGS

SEQ ID NO: 656 - protein, 15 aa, synthetic construct
GGGGSGGGGSGGGGS

SEQ ID NO: 657 - protein, 16 aa, synthetic construct
GGSGGSGSGGSGGSGS

SEQ ID NO: 658 - protein, 17 aa, synthetic construct
GGGGSGGGGSGGGGSGG

SEQ ID NO: 659 - protein, 18 aa, synthetic construct
SGGGSGGSGSGGSGGSGS

SEQ ID NO: 660 - protein, 19 aa, synthetic construct
EPEGGSGGSGSGGSGGSGS

SEQ ID NO: 661 - protein, 20 aa, synthetic construct
YGGSGGSGGSGSGGSGGSGS

SEQ ID NO: 662 - protein, 24 aa, synthetic construct
GGSGGSGSGGSGGSGSGGSGSGGS

SEQ ID NO: 663 - protein, 24 aa, synthetic construct
GSGGSGGSGGSGGSGSGGSGGSGS

SEQ ID NO: 664 - protein, 30 aa, synthetic construct
KSDELLGSGGSGSGSGGSEKAAKAEEAARK

SEQ ID NO: 665 - protein, 12 aa, synthetic construct
EKAAKAEEAARK

SEQ ID NO: 666 - protein, 26 aa, synthetic construct
DKIEEILSKIYHIENEIARIKKLIGE

SEQ ID NO: 667 - protein, 22 aa, synthetic construct
EKFHQIEKEFSEVEGRIQDLEK

SEQ ID NO: 668 - protein, 28 aa, synthetic construct
EDKIEEILSKIYHIENEIARIKKLIGEA

SEQ ID NO: 669 - protein, 29 aa, synthetic construct
GSGYIPEAPRDGQAYVRKDGEWVLLSTFL

SEQ ID NO: 670 - protein, 33 aa, synthetic construct
RMKQIEDKIEEILSKIYHIENEIARIKKLIGEA

SEQ ID NO: 671 - protein, 36 aa, synthetic construct
RMKQIEDKIEEILSKIYHIENEIARIKKLIGERGGR

SEQ ID NO: 672 - protein, 37 aa, synthetic construct
RMKQIEDKIEEILSKIYHIENEIARIKKLIGNRTGGR

SEQ ID NO: 673 - protein, 37 aa, synthetic construct
RMKQIEDKIENITSKIYHIENEIARIKKLIGNRTGGR

SEQ ID NO: 674 - protein, 37 aa, synthetic construct
RMKQIEDKIEEILSKIYNITNEIARIKKLIGNRTGGR

SEQ ID NO: 675 - protein, 37 aa, synthetic construct
RMKQIEDKIENITSKIYNITNEIARIKKLIGNRTGGR

SEQ ID NO: 676 - protein, 550 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 677 - protein, 549 aa, synthetic construct
MELLILKANAITTILTAVTFCFASGQNITEEFYQSTCSAVSKGYLSALRTGWYHSVITIELSNIKENKCNGTDAKVKLIKQELDKYKNAVTELQLLMQSTPACNNRARRELPRFMNYTLNNAKKTNVTLSKKRKRRFLGFLLGVGSACASGVAVSKVLHLEGEVNKIKSALLSTNKAWSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 678 - protein, 550 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 679 - protein, 550 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLCGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMCSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 680 - protein, 549 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 681 - protein, 550 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 682 - protein, 550 aa, synthetic construct
MELLILKANAITTILTAVTFCFASGQNITEEFYQSTCSAVSKGYLSALRTGWYTCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 683 - protein, 550 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 684 - protein, 550 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 685 - protein, 550 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLCGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEILAYVVQLPLYGVIDTPCWKLHTSPLCTTNTKEGSNICLTRTDRGWYCDNAGSVSFFPQAETCKVQSNRVFCDTMCSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSQFSASISQVNEKINQSREIIRAINIVRKIASEKSAIGGYIPEAPRDGQAYVRKDGEWVLLSTFL

SEQ ID NO: 686 - protein, 549 aa, synthetic construct
MELLILKANAITTILTAVTFCFASGQNITEEFYQSTCSAVSKGYLSALRTGWYHSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTIKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEILAYWQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 687 - protein, 549 aa, synthetic construct
MELLILKANAITTILTAVTFCFASGQNITEEFYQSTCSAVSKGYLSALRTGWYHCVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVCTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSSEFDASISQVNEKINQSREIIRAINIVRKIASEKSAIGGYIPEAPRDGQAYVRKDGEWVLLSTFL

SEQ ID NO: 688 - protein, 540 aa, synthetic construct
MELLILKAAITTILTAVTFCFASGQNITEEFYQSTCSAVSKGYLSALRTGWYTSCITIELSNIKENKCNGTDAVKLIKQELDKYKNAVTELQLLMQSTPATNNRARRELPRFMYTLNNAKKTVTLSKKRKRRFLGFLLGVGSAIASGVAVSKVLHLEGECNKIKSALLSTNKAVVSLSNGVSVLTSKVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEKSAIGGYIPEAPRDGQAYVRKDGEWVLLSTFL

SEQ ID NO: 689 - protein, 538 aa, synthetic construct
MELLILKAAITTILTAVTFCFASGQNITEEFYQSTCSAVSKGYLSALRTGWYTSVCTIELSNIKENKCNGTDAVKLIKQELDKYKNAVTELQLLMQSTPATNNRARRELPRFMYTLNNAKKTVTLSKKRKRRFLGFLLGVGSAIASGVAVSBVLHLEGEVKIKSALLSTNKAWSLSNGVSVLTCBVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEKSAIGGYIPEAPRDGQAYVRKDGEWVLLSTFL

SEQ ID NO: 690 - protein, 540 aa, synthetic construct
MELLILKAAITTILTAVTFCFASGQNITEEFYQSTCSAVSKGYLSALRTGWYTSVICIELSNIKENKCNGTDAVKLIKQELDKYKNAVTELQLLMQSTPATNNRARRELPRFMYTLNNAKKTVTLSKKRKRRFLGFLLGVGSAIASGVAVSKVLHLEGECNKIKSALLSTNKAVVSLSNGVSVLTSKVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEKSAIGGYIPEAPRDGQAYVRKDGEWVLLSTFL

SEQ ID NO: 691 - protein, 539 aa, synthetic construct
MELLILKAAITTILTAVTFCFASGQNITEEFYQSTCSAVSKGYLSALRTGWYTSVITIELSNIKENKCNGTDAVKLIKQELDKYKNAVTELQLLMQSTPATNNRARRELPRFMYTLNNAKKTVTLSKKRKRRFLGFLLGVGSAIASGVAVSBVLHLEGEVCKIKSALLSTNKAWSLSNGVSVLTSBVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEECLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEKSAIGGYIPEAPRDGQAYVRKDGEWVLLSTFL

SEQ ID NO: 692 - protein, 537 aa, synthetic construct
MELLILKAAITTILTAVTFCFASGQNITEEFYQSTCSAVSKGYLSALRTGWYTSVITIELSNIKENKCNGTDAKVKLIKQELDKYKNAVTELQLLMQSTPATIWRARRELPRFMYTLAKKTVTLSKKRKRRFLGFLLGVGSAIASGVAVSBVLHLEGEVKICSALLSTNKAWSLSNGVSVLTSBVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEECLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKIMTSKTDVSSSVITSLGAIVSCYGKTKCTASNKNRGIIKTFSNGCDYVSNKGVDTVSVGNTLYYVKQEGKSLYVKGEPIINFYDPLVFPSDEFDASISQVEKINQSREIIRAINIVRKIASEKSAIGGYIPEAPRDGQAYVRKDGEWVLLSTFL

SEQ ID NO: 693 - protein, 539 aa, synthetic construct
MELLILKAAITTILTAVTFCFASGQNITEEFYQSTCSAVSKGYLSALRTGWYTSVITIELSNIKENKCNGTDAVKLIKQELDKYKNAVTELQLLMQSTPATNNRARRELPRFMYTLNNAKKTVTLSKKRKRRFLGFLLGVGSAIASGVAVSKVLHLEGEVKIKSALLSTNKAVVSLSNGVSVLTSKVLDLKNYIDKQLLPIVKQSCSISNIETVIEFQQKNNRLLEITREFSVAGVTTPVSTYMLTNSELLSLINDMPITNDQKKLMSNNVQIVRQQSYSIMSIIKEEVLAYWQLPLYGVIDTPCWKLHTSPLCTTNTKEGSNICLTRTDRGWYCDNAGSVSFFPQAETCKVQSNRVFCDTMSLTLPSEVNLCNVDIFNPKYDCKICTSKTDVSSSVITSLGAIVSCYGKTKCTASNKNRGIIKTFSNGCDYVSNKGVDTVSVGNTLYYVKQEGKSLYVKGEPIINFYDPLVCPSDEFDASISQVEKINQSREIIRAINIVRKIASEKSAIGGYIPEAPRDGQAYVRKDGEWVLLSTFL

SEQ ID NO: 694 - protein, 549 aa, synthetic construct
METPAQLLFLLLLWLPDTTGFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 695 - protein, 549 aa, synthetic construct
METPAQLLFLLLLWLPDTTGFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 696 - protein, 550 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 697 - protein, 550 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 698 - protein, 527 aa, synthetic construct
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARQQQQRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 699 - protein, 520 aa, synthetic construct
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKFLGFLLGVGSAIASGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 700 - protein, 550 aa, synthetic construct; identical to SEQ ID NO: 697

SEQ ID NO: 701 - protein, 549 aa, synthetic construct
MELLILKANAITTILTAVTFCFASQNITEEFYQSTCSAVSKGYLSALRTGWYTSVITIELSNIKENKCNGTDAKVKLIKQELDKYKNAVTELQLLMQSTPATNNRARRELPRFMNYTLNNAKKINVILSKKRKRRFLGFLLGVGSAIASGVAVCKVLHLEGEVNKIKSALLSINKAVVSLSNGVSVLIFKVLDLKNYIDKQLLPILNKQSCSISNIETVIEFQQKNNRLLEITREFSVNAGVITPVSTYMLINSELLSLINDMPITNDQKKLMSNNVQIVRQQSYSIMCIIKEEVLAYVVQLPLYGVIDTPCWKLHISPLCTINTKEGSNICLTRIDRGWYCDNAGSVSFFPQAETCKVQSNRVFCDTMNSLTLPSEVNLCNVDIFNPKYDCKIMISKTDVSSSVITSLGAIVSCYGKTKCIASNKNRGIIKTFSNGCDYVSNKGVDTVSVGNTLYYVNKQEGKSLYVKGEPIINFYDPLVFPSDEFDASISQVNEKINQSREIIRAINIVRKIASEKSAIGGYIPEAPRDGQAYVRKDGEWVLLSTFL

SEQ ID NO: 702 - protein, 244 aa, synthetic construct
METDTLLLWVLLLWVPGSTGDYKDEKMEELFKKHKIVAVLRANSVEEAKKKALAVFLGGVDLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLHHHHHH

SEQ ID NO: 703 - protein, 244 aa, synthetic construct
METDTLLLWVLLLWVPGSTGDYKDEKMEELFKKHKIVAVLRANSVEEAKKDALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLHHHHHH

SEQ ID NO: 704 - protein, 244 aa, synthetic construct
METDTLLLWVLLLWVPGSTGDYKDEKMEELFKKHKIVAVLRANSVEEAKKNALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLHHHHHH

SEQ ID NO: 705 - protein, 244 aa, synthetic construct
METDTLLLWVLLLWVPGSTGDYKDEKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGSALVKGTPVEVAEKAKAFVEKIRGCTEQKLISEEDLHHHHHH

SEQ ID NO: 706 - protein, 244 aa, synthetic construct
METDTLLLWVLLLWVPGSTGDYKDEKMEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPVEVAEKAKAFVEKIRGCTEQKLISEEDLHHHHHH

SEQ ID NO: 707 - protein, 244 aa, synthetic construct
METDTLLLWVLLLWVPGSTGDYKDEKMEELFKKHKIVAVLRANSVEEAKKKALAVFLGGVDLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVQAVGVGEALNKGTPVEVAEKAKAFVEKIRGCTEQKLISEEDLHHHHHH

SEQ ID NO: 708 - protein, 239 aa, synthetic construct
METDTLLLWVLLLWVPGSTGDMEELFKEHKIVAVLRANSVEEAKKKALAVFLGGVHLIEITFTVPDADTVIKELSFLKEMGAIIGAGTVTSVEQAREAVESGAEFIVSPHLDEEISQFAKEEGVFYMPGVMTPTELVKAMKLGHTILKLFPGEVVGPQFVEAMKGPFPNVKFVPTGGVNLCNVAEWFEAGVLAVGVGSALVEGTPVEVAEKAKAFVEKIEGATEQKLISEEDLHHHHHH

SEQ ID NO: 709 - protein, 239 aa, synthetic construct
METDTLLLWVLLLWVPGSTGDMEELFKEHKIVAVLRANSVEEAKKKALAVFLGGVDLIEITFTVPDADTVIKELSFLKEMGAIIGAGTVTSVEQAREAVESGAEFIVSPHLDEEISQFAKEEGVFYMPGVMTPTELVKAMKLGHTILKLFPGEVVGPQFVEAMKGPFPNVKFVPTGGVNLDNVAEWFEAGVQAVGVGEALNEGTPVEVAEKAKAFVEKIEGATEQKLISEEDLHHHHHH

SEQ ID NO: 710 - protein, 39 aa, synthetic construct
LVFPSDEFDASISQVNEKINQSLAFIRRSDELLHNVNTG

SEQ ID NO: 711 - protein, 39 aa, synthetic construct
LVFPSDEFDASISQVNEKINQSLAFIRRSXXXXXXXXXX

SEQ ID NO: 712 - protein, 19 aa, synthetic construct
HHHHHHHHHHHHHHHHHHH

SEQ ID NO: 713 - protein, 20 aa, synthetic construct
ISQVNEKINQSLAFIRRSDE

SEQ ID NO: 714 - protein, 28 aa, synthetic construct
ISQVNEKINQSREIIRAINIVRKIASEK

SEQ ID NO: 715 - protein, 26 aa, synthetic construct
ISQVNEKINQSLAFIRRSDELLHNVN

SEQ ID NO: 716 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGDIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 717 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGDIGTTALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 718 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGDIGTISLGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 719 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGDIGTIASGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 720 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVDGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 721 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVDGTTALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 722 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVDGTISLGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 723 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVDGTIASGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 724 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGTIGTDALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 725 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGTIGTIDLGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 726 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGTIGTIADGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 727 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVTGTDALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 728 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVTGTIDLGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 729 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVTGTIADGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 730 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTDALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 731 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIDLGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 732 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIADGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 733 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERYYGGTTGTTSSGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 734 - protein, 710 aa, synthetic construct
QIDITKLQHVGVLVNSPKGMKISQNFETRYLILSLIPKIEDSNSCGDQQIKQYKRLLDRLIIPLYDGLKLQKDVIVTGGGSGGGATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 735 - protein, 712 aa, synthetic construct
QIDITKLQHVGVLVNSPKGMKISQNFETRYLILSLIPKIEDSNSCGDQQIKQYKRLLDRLIIPLYDGLKLQKDVIVTGGGSGSGGGATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 736 - protein, 714 aa, synthetic construct
QIDITKLQHVGVLVNSPKGMKISQNFETRYLILSLIPKIEDSNSCGDQQIKQYKRLLDRLIIPLYDGLKLQKDVIVTGGGSGGGSGGGATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 737 - protein, 716 aa, synthetic construct
QIDITKLQHVGVLVNSPKGMKISQNFETRYLILSLIPKIEDSNSCGDQQIKQYKRLLDRLIIPLYDGLKLQKDVIVTGGGSGGSGGSGGGATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 738 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVASDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 739 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVAPDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 740 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVASDPTDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 741 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVAPDPTDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 742 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVAKSPDDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 743 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITDNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 744 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITDNNSVASDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 745 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITDNNSVAPDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 746 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITDNNSVASDPTDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 747 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITDNNSVAPDPTDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 748 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEWESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 749 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEWESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPVDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 750 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEWESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPADISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 751 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEWESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISVELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 752 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLDHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIRISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 753 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLKHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 754 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVKLDPIDISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 755 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLDHEMESCLSGNISQCPRTTVTSDIVPRYAFVNGGVVANCITTTCTCNGIGNRINQPPDQGVKIITHKECNTIGINGMLFNTNKEGTLAFYTPDDITDNNSVASDPIRISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 756 - protein, 730 aa, synthetic construct
QIDITKLQHVGVLVNSPKGMKISQNFETRYLILSLIPKIEDSNSCGDQQIKQYKRLLDRLIIPLYDGLKLQKDVIVTNQESNENTDPRTERFFGGVIGTIALGVATSAQITAAVALVEAKQAKSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLDHEMESCLSGNISQCPRTTVTSDIVPRYAFVNGGVVANCITTTCTCNGIGNRINQPPDQGVKIITHKECNTIGINGMLFNTNKEGTLAFYTPDDITDNNSVAPDPIRISIELNKAKSDLEESKEWIRRSNQKLSAIEDKIEEILSKIYHIENEIARIKKLIGEAPGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATELEHHHHHH

SEQ ID NO: 757 - protein, 26 aa, synthetic construct
LQQNISSLEKALKKAEKDLEEVRRQL

SEQ ID NO: 758 - protein, 13 aa, synthetic construct
LESAMKTAMKIIS

SEQ ID NO: 759 - protein, 16 aa, synthetic construct
LQRTVDKLNSQIQALI

SEQ ID NO: 760 - protein, 22 aa, synthetic construct
LSKNVESLAKEVKKLEQKLNSL

SEQ ID NO: 761 - protein, 15 aa, synthetic construct
LKKAMETAIKRINKA

SEQ ID NO: 762 - protein, 24 aa, synthetic construct
LTANASENTARIEALERRIHELEL

SEQ ID NO: 763 - protein, 26 aa, synthetic construct
LSQTIKNLQDEVTKVTEELKKLVEQL

SEQ ID NO: 764 - protein, 21 aa, synthetic construct
LEKAAKKTLKIAKEESTKDKS

SEQ ID NO: 765 - protein, 22 aa, synthetic construct
LTENVTNLKKRLSEVEKVIKTL

SEQ ID NO: 766 - protein, 14 aa, synthetic construct
VNTTVRKLSEILAS

SEQ ID NO: 767 - protein, 19 aa, synthetic construct
LEKAIKKTLKIIRTELSIS

SEQ ID NO: 768 - protein, 18 aa, synthetic construct
LDNNITSLSERIHKLENL

SEQ ID NO: 769 - protein, 22 aa, synthetic construct
LSKNIEEIEKRLSELESTIKKL

SEQ ID NO: 770 - protein, 17 aa, synthetic construct
LESAIKKALTIIKQIWS

SEQ ID NO: 771 - protein, 18 aa, synthetic construct
IQESLQRLSERVEEIERR

SEQ ID NO: 772 - protein, 24 aa, synthetic construct
LDSDAESLADKVTALETRIKSIEA

SEQ ID NO: 773 - protein, 23 aa, synthetic construct
LDSAASRALKIAIELLRATESKK

SEQ ID NO: 774 - protein, 20 aa, synthetic construct
LNTQVKKLKDRIKKIEERLN

SEQ ID NO: 775 - protein, 14 aa, synthetic construct
LQKDVKSVETRLRT

SEQ ID NO: 776 - protein, 21 aa, synthetic construct
LEKAASKAIKISLKILKEILS

SEQ ID NO: 777 - protein, 26 aa, synthetic construct
LSSNVSNLRTDLNDLKKLVKKLIELL

SEQ ID NO: 778 - protein, 20 aa, synthetic construct
IQTNIKQNTERIDKIEKTLK

SEQ ID NO: 779 - protein, 11 aa, synthetic construct
LEKAIKEALKR

SEQ ID NO: 780 - protein, 24 aa, synthetic construct
IDKDIQKNTERINKIEKTIKSLIS

SEQ ID NO: 781 - protein, 20 aa, synthetic construct
LQRDVRKLEKRLTHVEEVLK

SEQ ID NO: 782 - protein, 17 aa, synthetic construct
LETAIKIALEIARKEIS

SEQ ID NO: 783 - protein, 24 aa, synthetic construct
ISENLKEAQERVDKIEKLLEKILR

SEQ ID NO: 784 - protein, 12 aa, synthetic construct
IDKSIKSLDTRL

SEQ ID NO: 785 - protein, 11 aa, synthetic construct
LDSAASYAIKV

SEQ ID NO: 786 - protein, 12 aa, synthetic construct
LDSDITAIQETL

SEQ ID NO: 787 - protein, 24 aa, synthetic construct
IDKSVDSLLTEVHAIRHEIDQLRS

SEQ ID NO: 788 - protein, 13 aa, synthetic construct
LEKAAKTALKIAS

SEQ ID NO: 789 - protein, 16 aa, synthetic construct
LQKQIKELRTVVKRLL

SEQ ID NO: 790 - protein, 12 aa, synthetic construct
LNTDVKQLQTSL

SEQ ID NO: 791 - protein, 23 aa, synthetic construct
LEKAAEEAVRRAIKLYKENLKKS

SEQ ID NO: 792 - protein, 12 aa, synthetic construct
LTRNIKDVKQAL

SEQ ID NO: 793 - protein, 20 aa, synthetic construct
INENISTITTEIKKIKEILL

SEQ ID NO: 794 - protein, 19 aa, synthetic construct
LETAASIAEKIARKLLKES

SEQ ID NO: 795 - protein, 12 aa, synthetic construct
ISSNITELKKTL

SEQ ID NO: 796 - protein, 26 aa, synthetic construct
LQDQISKLSNRVQRLERRLQEIERRL

SEQ ID NO: 797 - protein, 19 aa, synthetic construct
LESAIKKTLKIISKRNKDS

SEQ ID NO: 798 - protein, 21 aa, synthetic construct
IQENMERTKKWITKLIAKWKS

SEQ ID NO: 799 - protein, 20 aa, synthetic construct
LQEDVERLETLVREVQKQLE

SEQ ID NO: 800 - protein, 17 aa, synthetic construct
LEKAIKKATEIARKLIS

SEQ ID NO: 801 - protein, 19 aa, synthetic construct
ASKDMAEIIKTIKSLLKKS

SEQ ID NO: 802 - protein, 14 aa, synthetic construct
LNEQIESIEKDIAT

SEQ ID NO: 803 - protein, 19 aa, synthetic construct
LESAADKTMKKYKTEAKRS

SEQ ID NO: 804 - protein, 31 aa, synthetic construct
ATLDIEKTKRIMTSIALYVWTLIAKELKSKS

SEQ ID NO: 805 - protein, 26 aa, synthetic construct
LNKDLDELSSQLADLSARVEALQSTL

SEQ ID NO: 806 - protein, 21 aa, synthetic construct
LETALRIAIEITLQLLKKMAS

SEQ ID NO: 807 - protein, 33 aa, synthetic construct
IQETIKKVKKTAAEAITTQTRIWQKLKKSKSKS

SEQ ID NO: 808 - protein, 24 aa, synthetic construct
LDNSIKDLAKRVSDIESLVQKLLS

SEQ ID NO: 809 - protein, 17 aa, synthetic construct
LEKAIKITLKIIDIKLS

SEQ ID NO: 810 - protein, 31 aa, synthetic construct
LSEDIDKLEKKMSTIAKKLSKIEASKRKSSS

SEQ ID NO: 811 - protein, 26 aa, synthetic construct
IDSSISRNTDKIKELQQEIEKLQSSL

SEQ ID NO: 812 - protein, 15 aa, synthetic construct
LEKAAKKALEIASRS

SEQ ID NO: 813 - protein, 21 aa, synthetic construct
TNINVTKTEKKVEDLLKKLTS

SEQ ID NO: 814 - protein, 16 aa, synthetic construct
IQENVKKIEEILRSMS

SEQ ID NO: 815 - protein, 15 aa, synthetic construct
LSKTKAETLETVREL

SEQ ID NO: 816 - protein, 16 aa, synthetic construct
IDESVTRLAKILKKLI

SEQ ID NO: 817 - protein, 31 aa, synthetic construct
AQLTIETLARIVSTWYKQQAKKTATEEKRKS

SEQ ID NO: 818 - protein, 19 aa, synthetic construct
LEKTQSTTLTAAKTLIKST

SEQ ID NO: 819 - protein, 19 aa, synthetic construct
LETTRTKTITEVNTTISTT

SEQ ID NO: 820 - protein, 21 aa, synthetic construct
MNTQIDQIEKWLRDKEKKEQS

SEQ ID NO: 821 - protein, 15 aa, synthetic construct
LETTKKETLTEVTEA

SEQ ID NO: 822 - protein, 23 aa, synthetic construct
LEAVKTETLTAATTAINSALAKQ

SEQ ID NO: 823 - protein, 17 aa, synthetic construct
IDESTKKVKKIALDIAS

SEQ ID NO: 824 - protein, 17 aa, synthetic construct
LESTKAVTETEIKAEIN

SEQ ID NO: 825 - protein, 17 aa, synthetic construct
LKETQEKTITEVIKILN

SEQ ID NO: 826 - protein, 19 aa, synthetic construct
INESLKSLATDVKKLKSKI

SEQ ID NO: 827 - protein, 19 aa, synthetic construct
LNTTKTETISSIKKEIETM

SEQ ID NO: 828 - protein, 15 aa, synthetic construct
LTNTENNVLTRVKQS

SEQ ID NO: 829 - protein, 25 aa, synthetic construct
IDEDIDSLKKEVKKYIEKAEKDKKS

SEQ ID NO: 830 - protein, 25 aa, synthetic construct
LETAIKITLEIVLKILKEWEKRKSS

SEQ ID NO: 831 - protein, 13 aa, synthetic construct
LNALETRVLTAIN

SEQ ID NO: 832 - protein, 19 aa, synthetic construct
LDDTVRKALKWIKEVKKKS

SEQ ID NO: 833 - protein, 19 aa, synthetic construct
LEKAIKKTLKIIWTELSIS

SEQ ID NO: 834 - protein, 21 aa, synthetic construct
LTKLKEEVLEEVETMIRETAA

SEQ ID NO: 835 - protein, 25 aa, synthetic construct
LNEDIIKILQKLLTWITKTKQEKKS

SEQ ID NO: 836 - protein, 30 aa, synthetic construct
LVSTNAQLVKTIKLVIKAILTAIKEKKASS

SEQ ID NO: 837 - protein, 17 aa, synthetic construct
LDATSSRAIERVTTLLE

SEQ ID NO: 838 - protein, 31 aa, synthetic construct
ANLQIEKTKRKMTSIAKEVKTRIAKEEKSKS

SEQ ID NO: 839 - protein, 30 aa, synthetic construct
LADSSRDLSHVIQIMLETLETATKQKKKDS

SEQ ID NO: 840 - protein, 19 aa, synthetic construct
LDKVKDETVTIMTKYIQET

SEQ ID NO: 841 - protein, 17 aa, synthetic construct
TNLTVEKIWRYLMAVLS

SEQ ID NO: 842 - protein, 17 aa, synthetic construct
LQTLKEESTHLTKTLLS

SEQ ID NO: 843 - protein, 21 aa, synthetic construct
TQSQTEKILQWIKKFETKVKS

SEQ ID NO: 844 - protein, 25 aa, synthetic construct
TTKNTATIEKIVRSLLKEIKSERTR

SEQ ID NO: 845 - protein, 15 aa, synthetic construct
LEATHTRTLTTVTAA

SEQ ID NO: 846 - protein, 29 aa, synthetic construct
TTLTVTETIKELKSTDKKLKKYIKTVQSS

SEQ ID NO: 847 - protein, 23 aa, synthetic construct
IQEDVTRLKKIVEKLIRELQKIK

SEQ ID NO: 848 - protein, 19 aa, synthetic construct
LDTTKKETLTEAQETLERA

SEQ ID NO: 849 - protein, 20 aa, synthetic construct
VNKLKSELKTWIKQEANEKA

SEQ ID NO: 850 - protein, 24 aa, synthetic construct
TDTDVSKTLKMLLEFITREERSKR

SEQ ID NO: 851 - protein, 24 aa, synthetic construct
LVSSSKDLSEVIKWVREVVSKWIS

SEQ ID NO: 852 - protein, 28 aa, synthetic construct
LAETDATLQEVAKKLEEKIRTDIKREQS

SEQ ID NO: 853 - protein, 26 aa, synthetic construct
LRATTTNLSELAKELKKLKEHILRYQ

SEQ ID NO: 854 - protein, 32 aa, synthetic construct
LVQTNKTLDDTIKKLEKLERELRSRWDSERKS

SEQ ID NO: 855 - protein, 24 aa, synthetic construct
LTDNLDNLEERVKRLEEEVKKLKE

SEQ ID NO: 856 - protein, 36 aa, synthetic construct
LVNTTSDLSETQKKTKETATKLEQKTEKTLKYTKKK

SEQ ID NO: 857 - protein, 22 aa, synthetic construct
LIDTSKDLESLKKKLDELTKKS

SEQ ID NO: 858 - protein, 22 aa, synthetic construct
MNRLKKKLDQLWKILKEDKDKS

SEQ ID NO: 859 - protein, 19 aa, synthetic construct
LQATSDSLIKTQKLLKELI

SEQ ID NO: 860 - protein, 18 aa, synthetic construct
LQSTQKTLDALKKKVDKK

SEQ ID NO: 861 - protein, 28 aa, synthetic construct
VNKTQKKLKEIWKKLKKELTKERNTLKS

SEQ ID NO: 862 - protein, 28 aa, synthetic construct
LVATDRSLSALAEKCKKLKKKLEEDLKS

SEQ ID NO: 863 - protein, 21 aa, synthetic construct
LIKLSNSNTATIKKLDKLVKS

SEQ ID NO: 864 - protein, 26 aa, synthetic construct
LIATSKSLETTISILEEFLRRYKKKE

SEQ ID NO: 865 - protein, 32 aa, synthetic construct
LRQTTDQLNSVIKILKEIKEMLDKLLEKSKKS

SEQ ID NO: 866 - protein, 30 aa, synthetic construct
LISTNRNLAELAKKLDKTIEKASKDDSKKS

SEQ ID NO: 867 - protein, 20 aa, synthetic construct
LNDLSKDLEVAIKKIDKLES

SEQ ID NO: 868 - protein, 22 aa, synthetic construct
LVSSNSSLQELIKKVITLEKKS

SEQ ID NO: 869 - protein, 24 aa, synthetic construct
LRQTQSQLAKTQKLVTEILEKLTK

SEQ ID NO: 870 - protein, 19 aa, synthetic construct
LATTNRQLEELAKKFKEAS

SEQ ID NO: 871 - protein, 17 aa, synthetic construct
LQDVQSNLEKLIKEVKS

SEQ ID NO: 872 - protein, 20 aa, synthetic construct
LANTSKSLRIVIKEIRKLKS

SEQ ID NO: 873 - protein, 26 aa, synthetic construct
LQQLNLTLTELKKRTIKWYEETLKRT

SEQ ID NO: 874 - protein, 28 aa, synthetic construct
LQELTDDLAKLASKVETETRKERTKKKS

SEQ ID NO: 875 - protein, 19 aa, synthetic construct
LVDLSSQLKSLWKIMEKLS

SEQ ID NO: 876 - protein, 21 aa, synthetic construct
LVDTDKDLEDTIKKLEELTTK

SEQ ID NO: 877 - protein, 32 aa, synthetic construct
LVQLQKTNEALIKAITKKEEKSTRKERSERKS

SEQ ID NO: 878 - protein, 21 aa, synthetic construct
LVATQSNLRNVIKIIESQTRS

SEQ ID NO: 879 - protein, 20 aa, synthetic construct
LRKTNIDLTTLATKVEKALS

SEQ ID NO: 880 - protein, 20 aa, synthetic construct
LATTQKSLLETIKKVDKLTS

SEQ ID NO: 881 - protein, 20 aa, synthetic construct
LATTDEDLAALQTDIKRLKS

SEQ ID NO: 882 - protein, 24 aa, synthetic construct
LVTTSNDLTSVIKKLDKIVKKLQS

SEQ ID NO: 883 - protein, 34 aa, synthetic construct
LAATQNQLTELKKTTEKVIRTLKTKEEKKKQEKS

SEQ ID NO: 884 - protein, 24 aa, synthetic construct
LNKLDRSLDKVKKKVDKAITEIKS

SEQ ID NO: 885 - protein, 28 aa, synthetic construct
LIKLSSNLMDLARKTKEYWEKEERSKKS

SEQ ID NO: 886 - protein, 32 aa, synthetic construct
LATTTDNLTALKKEHEELLKEIKKEKEEKSRS

SEQ ID NO: 887 - protein, 20 aa, synthetic construct
LASSNQDLTELAKIVKSLIS

SEQ ID NO: 888 - protein, 32 aa, synthetic construct
LVDTSRNLEELAKKAKKFTEKLLSEIKKTKSD

SEQ ID NO: 889 - protein, 22 aa, synthetic construct
LLTTDKQLKELKKETEKLKKKV

SEQ ID NO: 890 - protein, 28 aa, synthetic construct
LRSTSRNLNNAIKRVLSWYKKKADEESS

SEQ ID NO: 891 - protein, 30 aa, synthetic construct
LAQTDKNLEKLATKTKQLEEKLEKEKKKSS

SEQ ID NO: 892 - protein, 18 aa, synthetic construct
LVDLQQNLEELAKEVKKK

SEQ ID NO: 893 - protein, 26 aa, synthetic construct
LQALTKQLTDLKKKLDSILTEQKRRS

SEQ ID NO: 894 - protein, 18 aa, synthetic construct
LVNLQTSLKDLKKKVDSK

SEQ ID NO: 895 - protein, 26 aa, synthetic construct
LVSQNLQLNKLAKRVKKYWEEVKSRS

SEQ ID NO: 896 - protein, 32 aa, synthetic construct
LNNLDRNLNNLKKKTEEIATDLEKKWRKMSKS

SEQ ID NO: 897 - protein, 30 aa, synthetic construct
LILTTNTLNNTITIMKKIEEKLKADKKKSS

SEQ ID NO: 898 - protein, 19 aa, synthetic construct
LNDLTKNLSKTQKLLKELI

SEQ ID NO: 899 - protein, 18 aa, synthetic construct
LAATTAQLTKTIKEMKEK

SEQ ID NO: 900 - protein, 22 aa, synthetic construct
LQATTRDLDDLKKKVDTLEKQS

SEQ ID NO: 901 - protein, 20 aa, synthetic construct
LNQVDRSLKELESELKSRLS

SEQ ID NO: 902 - protein, 22 aa, synthetic construct
LNALSTDVDDVIKKLDEALSRI

SEQ ID NO: 903 - protein, 17 aa, synthetic construct
LRTVDSNLNSLAKKLDS

SEQ ID NO: 904 - protein, 24 aa, synthetic construct
LVTTDQQLTSLAKQTKKLEDELRS

SEQ ID NO: 905 - protein, 32 aa, synthetic construct
LVRTTQDLEDLAKRTKTWYDILAKILASNQKS

SEQ ID NO: 906 - protein, 16 aa, synthetic construct
LARTNNDLEALAKYVS

SEQ ID NO: 907 - protein, 34 aa, synthetic construct
LVITQRTLDDVAKRAESTIRDLKETKKKQKKEKS

SEQ ID NO: 908 - protein, 21 aa, synthetic construct
LQNVQNNLNTLKTKIEQILKS

SEQ ID NO: 909 - protein, 26 aa, synthetic construct
LVHTTESLKLLKKRLEDYIKTQKAKS

SEQ ID NO: 910 - protein, 30 aa, synthetic construct
LRQLNATLSETIKELKSHLTTLKIEKSKKS

SEQ ID NO: 911 - protein, 36 aa, synthetic construct
LVTTTNNLKKTAKIALTVEKILTTRDKQKKKKDEKS

SEQ ID NO: 912 - protein, 36 aa, synthetic construct
LNELDANLQATIKTTEKALKIILKRIKKALAEQKSS

SEQ ID NO: 913 - protein, 22 aa, synthetic construct
LNSLDRTLDNLKKKVDEATKTT

SEQ ID NO: 914 - protein, 26 aa, synthetic construct
LVTTSRNLDVLASDVSSMKATEEKKS

SEQ ID NO: 915 - protein, 21 aa, synthetic construct
LVSSQIDLDDLIKKTDALEKS

SEQ ID NO: 916 - protein, 28 aa, synthetic construct
LIELNNDLEELKKKLEEILASIEKKEKS

SEQ ID NO: 917 - protein, 24 aa, synthetic construct
LVATQTNLALVIKKVETIASKLKS

SEQ ID NO: 918 - protein, 19 aa, synthetic construct
LIATNKNLSKLKKKLEKIL

SEQ ID NO: 919 - protein, 19 aa, synthetic construct
LVRTQESLNELKEKLDRYI

SEQ ID NO: 920 - protein, 18 aa, synthetic construct
LIQLSRDLSDLKKTLEKR

SEQ ID NO: 921 - protein, 24 aa, synthetic construct
LASTNKSLSILAKKTKEAIDRIRS

SEQ ID NO: 922 - protein, 24 aa, synthetic construct
LVTTDKTLQETQKQLETLAKKIKS

SEQ ID NO: 923 - protein, 17 aa, synthetic construct
LAETSKNLKSLIKKFNS

SEQ ID NO: 924 - protein, 26 aa, synthetic construct
LAQTSKTLSETIKKVDKSTKSTEKKS

SEQ ID NO: 925 - protein, 26 aa, synthetic construct
LNNATIQLERVIKDLKKTKEKQKRSS

SEQ ID NO: 926 - protein, 21 aa, synthetic construct
LNKVKEDIEKLEERVHAIEKK

SEQ ID NO: 927 - protein, 23 aa, synthetic construct
LNKVKERVKENEKIITKIQKTLD

SEQ ID NO: 928 - protein, 24 aa, synthetic construct
LNKLAKEVKTILKKLSKKLSSLES

SEQ ID NO: 929 - protein, 21 aa, synthetic construct
LNKVKNRVEKLEETLTRLINA

SEQ ID NO: 930 - protein, 31 aa, synthetic construct
LNKVKTEVKEITKKVRELEERLRKVEEVVKS

SEQ ID NO: 931 - protein, 23 aa, synthetic construct
LNKVKSKTETMAEKMRSKETATS

SEQ ID NO: 932 - protein, 27 aa, synthetic construct
LNKVKDDLESVNKRVSEIEHELHEIKA

SEQ ID NO: 933 - protein, 25 aa, synthetic construct
LNKVKSDVRDLEERLHKLETRLEEI

SEQ ID NO: 934 - protein, 23 aa, synthetic construct
LNKVKSKTETYIKETRSKETATS

SEQ ID NO: 935 - protein, 29 aa, synthetic construct
LNKVKEEVKELTEEIHELREEVEALKEEL

SEQ ID NO: 936 - protein, 21 aa, synthetic construct
LNKVKSEVKKLKERLEELEAR

SEQ ID NO: 937 - protein, 22 aa, synthetic construct
MNRLKSKLDKLLKELKEDKDKS

SEQ ID NO: 938 - protein, 25 aa, synthetic construct
LNKVKQQVEKLIERLHRLENKLAEA

SEQ ID NO: 939 - protein, 23 aa, synthetic construct
LNKVKEKVDKIQENIDAIKTILD

SEQ ID NO: 940 - protein, 23 aa, synthetic construct
LNKVKKETKTFIKEVRSKETATS

SEQ ID NO: 941 - protein, 23 aa, synthetic construct
LNKVKTELHKLKERVRDIEKKLA

SEQ ID NO: 942 - protein, 27 aa, synthetic construct
LNKVKNEVSELEKRTTKIESTIKTLIE

SEQ ID NO: 943 - protein, 21 aa, synthetic construct
LNKVKSKTETYIKEVRSKETA

SEQ ID NO: 944 - protein, 25 aa, synthetic construct
LNKVKKEVEELRKRLKKLEEKLTSV

SEQ ID NO: 945 - protein, 23 aa, synthetic construct
LNKVKDKVEKDTKKIKEIEHELA

SEQ ID NO: 946 - protein, 16 aa, synthetic construct
LNSLQRDHEKLIKEVK

SEQ ID NO: 947 - protein, 27 aa, synthetic construct
LNKVKKKVSELEKQVTEIEKILTEIRA

SEQ ID NO: 948 - protein, 21 aa, synthetic construct
LNKVKKDLKELSEKVHELLNS

SEQ ID NO: 949 - protein, 21 aa, synthetic construct
LNSLQKSLVELKKKLDELEKR

SEQ ID NO: 950 - protein, 21 aa, synthetic construct
LNKVKERLHKLEESVKQLKKA

SEQ ID NO: 951 - protein, 25 aa, synthetic construct
LNKVKKRLEELEEKLDRLEHIVHLL

SEQ ID NO: 952 - protein, 24 aa, synthetic construct
LNKLNRQLAALAKKTKELEKKIKS

SEQ ID NO: 953 - protein, 19 aa, synthetic construct
LNKVKSDVENLKEKINKII

SEQ ID NO: 954 - protein, 19 aa, synthetic construct
LNKVKENVEEIEHKVKEIE

SEQ ID NO: 955 - protein, 13 aa, synthetic construct
LENLKNTVESIIN

SEQ ID NO: 956 - protein, 25 aa, synthetic construct
LNKVKDDVRTIKKELEELKQLVKNL

SEQ ID NO: 957 - protein, 31 aa, synthetic construct
LNKVKKEVNELNKRIRSLEQRVEKLERALKK

SEQ ID NO: 958 - protein, 13 aa, synthetic construct
LERIRTEVTQASA

SEQ ID NO: 959 - protein, 19 aa, synthetic construct
LNKVKERVKSLEKQLKTLL

SEQ ID NO: 960 - protein, 27 aa, synthetic construct
LNKVKKDLKKTKENLKEVEEKVKELLS

SEQ ID NO: 961 - protein, 19 aa, synthetic construct
LNKVKKDVTYLKTEVAQLQ

SEQ ID NO: 962 - protein, 31 aa, synthetic construct
LNKVKTRVEEIERKISSLEKEVEDIRRSLQQ

SEQ ID NO: 963 - protein, 25 aa, synthetic construct
LNKVKKELEELLQKVKDLEEKVETL

SEQ ID NO: 964 - protein, 29 aa, synthetic construct
LNKVKKEVKELKERLDHVEKRLKEVEEKL

SEQ ID NO: 965 - protein, 31 aa, synthetic construct
LNKVKNKLEKVESQVHRLENRIEKIERLLKS

SEQ ID NO: 966 - protein, 27 aa, synthetic construct
LNKVKKMVESLESKVTKLEKTVKELLT

SEQ ID NO: 967 - protein, 21 aa, synthetic construct
LNKVKEDVASLKKEVEKIIKA

SEQ ID NO: 968 - protein, 29 aa, synthetic construct
LNKVKRDVEQLRQELNSLSKRVHKIEEAL

SEQ ID NO: 969 - protein, 21 aa, synthetic construct
LNKVKSELDKLKKKVEHIENS

SEQ ID NO: 970 - protein, 19 aa, synthetic construct
LNKVKNSLDKVEKKVTSLI

SEQ ID NO: 971 - protein, 21 aa, synthetic construct
LNKVKSAVTHLTKEVTKLKEL

SEQ ID NO: 972 - protein, 23 aa, synthetic construct
LNKVKKDVEKLKKRISHIEKLLS

SEQ ID NO: 973 - protein, 27 aa, synthetic construct
LNKVKKKVESLERKVSKLENEIKTIID

SEQ ID NO: 974 - protein, 23 aa, synthetic construct
LNKVKKDLNDAKKRISHIEKVLN

SEQ ID NO: 975 - protein, 23 aa, synthetic construct
LNKVKKEVRKLEHEIHEIKKRLA

SEQ ID NO: 976 - protein, 25 aa, synthetic construct
LNKVKKKVSELEKRVDHIEHRLKQI

SEQ ID NO: 977 - protein, 29 aa, synthetic construct
LNKVKADLTTLESKQSEIERRVAKIEHAL

SEQ ID NO: 978 - protein, 24 aa, synthetic construct
LNKLAKEVKTILKELSKKLSSLES

SEQ ID NO: 979 - protein, 29 aa, synthetic construct
LNKVKKKVEKIEKEIEKLKRELETVKREI

SEQ ID NO: 980 - protein, 29 aa, synthetic construct
LNKVKEEVEKLERETKKLSHEIKKIKETL

SEQ ID NO: 981 - protein, 31 aa, synthetic construct
LNKVKSEVSELKTKVQTLETRIKKIEHELKL

SEQ ID NO: 982 - protein, 7 aa, synthetic construct
DRIKRAL

SEQ ID NO: 983 - protein, 19 aa, synthetic construct
ERLEKALQTLTKAMKKTLS

SEQ ID NO: 984 - protein, 12 aa, synthetic construct
EKIERAIRKLES

SEQ ID NO: 985 - protein, 8 aa, synthetic construct
ERIDKAIS

SEQ ID NO: 986 - protein, 9 aa, synthetic construct
TKIEKAITS

SEQ ID NO: 987 - protein, 12 aa, synthetic construct
ERIDSAIKKALS

SEQ ID NO: 988 - protein, 17 aa, synthetic construct
EEIEKAIKILKKILKES

SEQ ID NO: 989 - protein, 20 aa, synthetic construct
EEIKKAIKILKKILKELSSS

SEQ ID NO: 990 - protein, 13 aa, synthetic construct
EKLKRATEKARKS

SEQ ID NO: 991 - protein, 17 aa, synthetic construct
ERIKKAIKTAIEAMQKS

SEQ ID NO: 992 - protein, 21 aa, synthetic construct
ERIKKAIEIMLSWKKALEKNS

SEQ ID NO: 993 - protein, 13 aa, synthetic construct
ETILRAIKKAQKS

SEQ ID NO: 994 - protein, 17 aa, synthetic construct
EKIEKILKELEKEKQSR

SEQ ID NO: 995 - protein, 9 aa, synthetic construct
DRIERASKS

SEQ ID NO: 996 - protein, 8 aa, synthetic construct
EKLAQAVS

SEQ ID NO: 997 - protein, 17 aa, synthetic construct
EYIEKAIKAAQETIKKL

SEQ ID NO: 998 - protein, 21 aa, synthetic construct
EKITKAIKIAKELKKLIESML

SEQ ID NO: 999 - protein, 13 aa, synthetic construct
EEIKRAIEALRKR

SEQ ID NO: 1000 - protein, 17 aa, synthetic construct
ERIEKILKELEKEKQSR

SEQ ID NO: 1001 - protein, 21 aa, synthetic construct
EKITKAIKIAKELLKKIESML

SEQ ID NO: 1002 - protein, 14 aa, synthetic construct
ERTEKAIKITLTIS

SEQ ID NO: 1003 - protein, 8 aa, synthetic construct
EIIKQAIS

SEQ ID NO: 1004 - protein, 21 aa, synthetic construct
EKLKKAIEQMLTVKKITEKWS

SEQ ID NO: 1005 - protein, 14 aa, synthetic construct
EKITKAIEEMKKQS

SEQ ID NO: 1006 - protein, 17 aa, synthetic construct
EAIERAIKDMLTAKKQS

SEQ ID NO: 1007 - protein, 9 aa, synthetic construct
ERIDEAIKR

SEQ ID NO: 1008 - protein, 14 aa, synthetic construct
EKLEKAMEETKKLS

SEQ ID NO: 1009 - protein, 17 aa, synthetic construct
EEILRAIKTARTESKKT

SEQ ID NO: 1010 - protein, 9 aa, synthetic construct
QKILDAIKS

SEQ ID NO: 1011 - protein, 15 aa, synthetic construct
ERIKSAIKKLESQES

SEQ ID NO: 1012 - protein, 18 aa, synthetic construct
EKIKKAIEKAESIIQSIS

SEQ ID NO: 1013 - protein, 9 aa, synthetic construct
ERIESAIKS

SEQ ID NO: 1014 - protein, 15 aa, synthetic construct
EKIKSALELALRLAK

SEQ ID NO: 1015 - protein, 18 aa, synthetic construct
EEIDKAIKILKKILKELS

SEQ ID NO: 1016 - protein, 9 aa, synthetic construct
ERITKALQS

SEQ ID NO: 1017 - protein, 8 aa, synthetic construct
ERIERAIR

SEQ ID NO: 1018 - protein, 18 aa, synthetic construct
EKTKKAIKITEEIYKKLS

SEQ ID NO: 1019 - protein, 9 aa, synthetic construct
ERIEEAIRR

SEQ ID NO: 1020 - protein, 15 aa, synthetic construct
ERIEEAIRRASKNDG

SEQ ID NO: 1021 - protein, 18 aa, synthetic construct
ERIKKAIKTANEHLSKVN

SEQ ID NO: 1022 - protein, 10 aa, synthetic construct
DRIKKALSKL

SEQ ID NO: 1023 - protein, 15 aa, synthetic construct
EKIKQAIELTLKLAS

SEQ ID NO: 1024 - protein, 19 aa, synthetic construct
EKIERAIKWIEDLLKKEKS

SEQ ID NO: 1025 - protein, 10 aa, synthetic construct
DKIKRAITKT

SEQ ID NO: 1026 - protein, 8 aa, synthetic construct
DKIKRAIS

SEQ ID NO: 1027 - protein, 19 aa, synthetic construct
EEIKKAIKEARKAIEKLKS

SEQ ID NO: 1028 - protein, 10 aa, synthetic construct
ESIKEAIKQS

SEQ ID NO: 1029 - protein, 16 aa, synthetic construct
EKIKRAIDIVEKLTQS

SEQ ID NO: 1030 - protein, 19 aa, synthetic construct
EEIDKAIKEARKAIEKLKS

SEQ ID NO: 1031 - protein, 11 aa, synthetic construct
EKIKQTMKKAS

SEQ ID NO: 1032 - protein, 16 aa, synthetic construct
ESIERAIKSTKEAIKS

SEQ ID NO: 1033 - protein, 19 aa, synthetic construct
EKISQAIDKTTKIILSIES

SEQ ID NO: 1034 - protein, 8 aa, synthetic construct
EKLTQAAS

SEQ ID NO: 1035 - protein, 16 aa, synthetic construct
ERIKRALEKLTKATKS

SEQ ID NO: 1036 - protein, 19 aa, synthetic construct
ERIKQAIKKVEETLKRLKS

SEQ ID NO: 1037 - protein, 11 aa, synthetic construct
EKILQAIRLAS

SEQ ID NO: 1038 - protein, 16 aa, synthetic construct
EKIKQAIEYMLKVAKS

SEQ ID NO: 1039 - protein, 8 aa, synthetic construct
DRIKRALS

SEQ ID NO: 1040 - protein, 11 aa, synthetic construct
TKIAEAIKRTS

SEQ ID NO: 1041 - protein, 12 aa, synthetic construct
EKIERAIKKASS

SEQ ID NO: 1042 - protein, 11 aa, synthetic construct
ERIKNAIKKME

SEQ ID NO: 1043 - protein, 11 aa, synthetic construct
ERINQALKKAD

SEQ ID NO: 1044 - protein, 12 aa, synthetic construct
EKIERAIKYALS

SEQ ID NO: 1045 - protein, 12 aa, synthetic construct
EKIERAIKKAQS

SEQ ID NO: 1046 - protein, 8 aa, synthetic construct
ERILSALS

SEQ ID NO: 1047 - protein, 17 aa, synthetic construct
QKIQDAVEELQTLMQKL

SEQ ID NO: 1048 - protein, 8 aa, synthetic construct
DRSERAQK

SEQ ID NO: 1049 - protein, 12 aa, synthetic construct
EEIKKETKRIRS

SEQ ID NO: 1050 - protein, 17 aa, synthetic construct
EELKKAASKAKEEIKRS

SEQ ID NO: 1051 - protein, 18 aa, synthetic construct
DKASKAIEYAERDAKSKS

SEQ ID NO: 1052 - protein, 12 aa, synthetic construct
EKMTKKANTAES

SEQ ID NO: 1053 - protein, 17 aa, synthetic construct
EEIKTIISILKELEKRS

SEQ ID NO: 1054 - protein, 20 aa, synthetic construct
SEIKKVITETRKITKKIKSS

SEQ ID NO: 1055 - protein, 12 aa, synthetic construct
EKMTKKANDAES

SEQ ID NO: 1056 - protein, 17 aa, synthetic construct
ETLKKQASKAEELEKRS

SEQ ID NO: 1057 - protein, 20 aa, synthetic construct
DKLTRTAQKAKTLIEETKKS

SEQ ID NO: 1058 - protein, 13 aa, synthetic construct
EEIDTLAKELKES

SEQ ID NO: 1059 - protein, 17 aa, synthetic construct
SRLKAELKKLKEILKKS

SEQ ID NO: 1060 - protein, 20 aa, synthetic construct
DKLTRIAQKALTLIEETKKS

SEQ ID NO: 1061 - protein, 13 aa, synthetic construct
IKIKTAAKQAKKK

SEQ ID NO: 1062 - protein, 18 aa, synthetic construct
EETKQAIKLVKKDYKEKS

SEQ ID NO: 1063 - protein, 21 aa, synthetic construct
SKIETAIKKLIEKERKTRAKK

SEQ ID NO: 1064 - protein, 13 aa, synthetic construct
ERIKETNKATKQK

SEQ ID NO: 1065 - protein, 18 aa, synthetic construct
EIIKQEIKKTQTFIKKVS

SEQ ID NO: 1066 - protein, 21 aa, synthetic construct
ERIKKTAKIAQKLYKTLKSQS

SEQ ID NO: 1067 - protein, 13 aa, synthetic construct
AKIETAIRKTIES

SEQ ID NO: 1068 - protein, 18 aa, synthetic construct
ETIKREIKKTREMTKKLL

SEQ ID NO: 1069 - protein, 21 aa, synthetic construct
ERIDKTAKIAQKLYKTLKSQS

SEQ ID NO: 1070 - protein, 13 aa, synthetic construct
SRIKAMIKKILKS

SEQ ID NO: 1071 - protein, 11 aa, synthetic construct
SRLKKAADKAS

SEQ ID NO: 1072 - protein, 9 aa, synthetic construct
ETIEKKLQS

SEQ ID NO: 1073 - protein, 14 aa, synthetic construct
ERLKKAAEIVERQT

SEQ ID NO: 1074 - protein, 11 aa, synthetic construct
ERLDKDAKTAK

SEQ ID NO: 1075 - protein, 7 aa, synthetic construct
SKIKKDL

SEQ ID NO: 1076 - protein, 14 aa, synthetic construct
ETIKKIIEEILSRS

SEQ ID NO: 1077 - protein, 12 aa, synthetic construct
DKLKRTAEKAKS

SEQ ID NO: 1078 - protein, 10 aa, synthetic construct
ERLERHLRSR

SEQ ID NO: 1079 - protein, 14 aa, synthetic construct
ETLEKVAKEVTKIS

SEQ ID NO: 1080 - protein, 12 aa, synthetic construct
EEIKTLAKELKE

SEQ ID NO: 1081 - protein, 10 aa, synthetic construct
IRTKQAIKSA

SEQ ID NO: 1082 - protein, 14 aa, synthetic construct
DELKRVITDLRKLK

SEQ ID NO: 1083 - protein, 12 aa, synthetic construct
ESSKKAQKQAKS

SEQ ID NO: 1084 - protein, 11 aa, synthetic construct
SRIKKILSEAS

SEQ ID NO: 1085 - protein, 15 aa, synthetic construct
EKILTAIKIALAAVS

SEQ ID NO: 1086 - protein, 16 aa, synthetic construct
DRLIKVAEKTSKMLKS

SEQ ID NO: 1087 - protein, 11 aa, synthetic construct
ETIKKLLKKAM

SEQ ID NO: 1088 - protein, 15 aa, synthetic construct
ERLDKTAKETKEYLS

SEQ ID NO: 1089 - protein, 16 aa, synthetic construct
DRLKKMLEKTSKMLKS

SEQ ID NO: 1090 - protein, 11 aa, synthetic construct
EKIKQIARLAS

SEQ ID NO: 1091 - protein, 15 aa, synthetic construct
DKIKKAVSWVLAVKS

SEQ ID NO: 1092 - protein, 16 aa, synthetic construct
ETIEKKLKTIESRLKS

SEQ ID NO: 1093 - protein, 11 aa, synthetic construct
EKIEQTRRLAS

SEQ ID NO: 1094 - protein, 15 aa, synthetic construct
EKLEKLERKTRQKDS

SEQ ID NO: 1095 - protein, 16 aa, synthetic construct
ETTKKAIELLKKLYKS

SEQ ID NO: 1096 - protein, 18 aa, synthetic construct
REIETAIKKAKEFIKTIK

SEQ ID NO: 1097 - protein, 15 aa, synthetic construct
EAIERTLKTIDKKVS

SEQ ID NO: 1098 - protein, 16 aa, synthetic construct
EDLKKTAAEAKKHIKS

SEQ ID NO: 1099 - protein, 21 aa, synthetic construct
RKTEEALRRADTIIKQLASKS

SEQ ID NO: 1100 - protein, 15 aa, synthetic construct
EELKKVAKEAKKAIS

SEQ ID NO: 1101 - protein, 17 aa, synthetic construct
ETIKKHIEIAIKFIKEV

SEQ ID NO: 1102 - protein, 13 aa, synthetic construct
AETKKAIERAREL

SEQ ID NO: 1103 - protein, 15 aa, synthetic construct
AKIEKTLKKLKTEDS

SEQ ID NO: 1104 - protein, 28 aa, synthetic construct
NTVRKTIETVNSLEKELKELRTEVDRLL

SEQ ID NO: 1105 - protein, 14 aa, synthetic construct
KEAKKAIETAKKLS

SEQ ID NO: 1106 - protein, 15 aa, synthetic construct
ARIKKTIEIVLTQTS

SEQ ID NO: 1107 - protein, 24 aa, synthetic construct
KEIRNTVKKVRTIEKRLNKLETSL

SEQ ID NO: 1108 - protein, 18 aa, synthetic construct
REIKDAIKKAKEFIKTIK

SEQ ID NO: 1109 - protein, 18 aa, synthetic construct
REVKEAIKIIKKILKKQS

SEQ ID NO: 1110 - protein, 22 aa, synthetic construct
KLVKKVIKETHEIKKKLEDLLK

SEQ ID NO: 1111 - protein, 21 aa, synthetic construct
QTTEEQIKTLTERVESIEKEG

SEQ ID NO: 1112 - protein, 10 aa, synthetic construct
QKILDEIKKT

SEQ ID NO: 1113 - protein, 20 aa, synthetic construct
IRWEANAKKAETEIKKLSES

SEQ ID NO: 1114 - protein, 29 aa, synthetic construct
QEIDKKLEYLEERVHDLEERLESLVQQLQ

SEQ ID NO: 1115 - protein, 11 aa, synthetic construct
ETILTTNKRAN

SEQ ID NO: 1116 - protein, 10 aa, synthetic construct
EITDRKNKKA

SEQ ID NO: 1117 - protein, 25 aa, synthetic construct
QNVEDRLEANEKAISHIEQLIDQLI

SEQ ID NO: 1118 - protein, 11 aa, synthetic construct
QIIQDTIKKMS

SEQ ID NO: 1119 - protein, 10 aa, synthetic construct
EIAKQLMTKA

SEQ ID NO: 1120 - protein, 25 aa, synthetic construct
QNIEDRVEDNDDKVAELKEELEAIK

SEQ ID NO: 1121 - protein, 13 aa, synthetic construct
IKIKQQIKRLDEK

SEQ ID NO: 1122 - protein, 26 aa, synthetic construct
RAIKETQKRTTVLEEDLKRVKELLKS

SEQ ID NO: 1123 - protein, 27 aa, synthetic construct
QNVEDRLEELESRIKKIEEEIEEIKKD

SEQ ID NO: 1124 - protein, 13 aa, synthetic construct
EYLLAVAETLNRR

SEQ ID NO: 1125 - protein, 16 aa, synthetic construct
RKATETIKKFEESEKS

SEQ ID NO: 1126 - protein, 27 aa, synthetic construct
QNIEEDLESLKERIHRLESEVQNLLER

SEQ ID NO: 1127 - protein, 13 aa, synthetic construct
EYILTAIKIMLTR

SEQ ID NO: 1128 - protein, 15 aa, synthetic construct
RKWNESSKKVQEQDS

SEQ ID NO: 1129 - protein, 23 aa, synthetic construct
QRTEKRINDLESRVARIEEVLSL

SEQ ID NO: 1130 - protein, 8 aa, synthetic construct
EILTQQAS

SEQ ID NO: 1131 - protein, 15 aa, synthetic construct
RRTLTAITRVERKDS

SEQ ID NO: 1132 - protein, 25 aa, synthetic construct
QETEDTLESLSQEVEKLRETVEKLT

SEQ ID NO: 1133 - protein, 16 aa, synthetic construct
QILLDAMTNTERALRS

SEQ ID NO: 1134 - protein, 17 aa, synthetic construct
AKTEEAYQRTIKTQQKL

SEQ ID NO: 1135 - protein, 23 aa, synthetic construct
QNILDRINENEQRVSVLERTLAQ

SEQ ID NO: 1136 - protein, 22 aa, synthetic construct
QSIQATTSRVDAIEAKVKHLEA

SEQ ID NO: 1137 - protein, 19 aa, synthetic construct
EIWETNTERSIKAVLSIQS

SEQ ID NO: 1138 - protein, 31 aa, synthetic construct
QSIEDSLSTLNTKINKLKKEVESLKREVEEL

SEQ ID NO: 1139 - protein, 26 aa, synthetic construct
KYISNRIKENTDQIKKLEERVTELEA

SEQ ID NO: 1140 - protein, 19 aa, synthetic construct
AKIETTKKITEELLDRAIK

SEQ ID NO: 1141 - protein, 18 aa, synthetic construct
AKAEHAIKFALSEEKSRS

SEQ ID NO: 1142 - protein, 24 aa, synthetic construct
LEIRQTSKRVESLERRVTQVERDR

SEQ ID NO: 1143 - protein, 24 aa, synthetic construct
QAIRETQDEVKNLNKRINKIVTSI

SEQ ID NO: 1144 - protein, 19 aa, synthetic construct
EIWETNTERSEKKVKSIQS

SEQ ID NO: 1145 - protein, 30 aa, synthetic construct
VTINNMISSNTNEISSLQDRVKHIEDTLAL

SEQ ID NO: 1146 - protein, 17 aa, synthetic construct
REIIRAINIVRKIASEK

SEQ ID NO: 1147 - protein, 19 aa, synthetic construct
RKTLETIEWVEKVIKKQRS

SEQ ID NO: 1148 - protein, 13 aa, synthetic construct
RTLLETAEIVTRS

SEQ ID NO: 1149 - protein, 20 aa, synthetic construct
AKLKETTERTEKIEKKIKDS

SEQ ID NO: 1150 - protein, 17 aa, synthetic construct
ALWLEAAKYVKQAREKS

SEQ ID NO: 1151 - protein, 11 aa, synthetic construct
RETAKAVSAVK

SEQ ID NO: 1152 - protein, 20 aa, synthetic construct
DELARAATLAKQLITKIKKS

SEQ ID NO: 1153 - protein, 17 aa, synthetic construct
RKTEKAIRLVLKWLKES

SEQ ID NO: 1154 - protein, 13 aa, synthetic construct
RTLLETAEIVKRS

SEQ ID NO: 1155 - protein, 21 aa, synthetic construct
EELAQTARLAKAYLKELKSRS

SEQ ID NO: 1156 - protein, 20 aa, synthetic construct
RDTLKAIEQTKRYLEELKKS

SEQ ID NO: 1157 - protein, 13 aa, synthetic construct
RKLDKAAEYVEKS

SEQ ID NO: 1158 - protein, 12 aa, synthetic construct
EYLAQVAEKVDK

SEQ ID NO: 1159 - protein, 18 aa, synthetic construct
RSWDIAAKFVKTVLSNQS

SEQ ID NO: 1160 - protein, 14 aa, synthetic construct
RKLETAAEKLKQTE

SEQ ID NO: 1161 - protein, 14 aa, synthetic construct
EKQKKINEMATKVT

SEQ ID NO: 1162 - protein, 18 aa, synthetic construct
RKTLEATEIAKKLAEDRS

SEQ ID NO: 1163 - protein, 14 aa, synthetic construct
RLMLEAVKIAQSQS

SEQ ID NO: 1164 - protein, 14 aa, synthetic construct
EYLKKVAEIVNKIS

SEQ ID NO: 1165 - protein, 20 aa, synthetic construct
LEILKAAKEAKKLIEDLRRS

SEQ ID NO: 1166 - protein, 15 aa, synthetic construct
RETKEAAESVKQMES

SEQ ID NO: 1167 - protein, 14 aa, synthetic construct
TETKKAIEIALKIS

SEQ ID NO: 1168 - protein, 20 aa, synthetic construct
KELLDAAKAVKKMLEKEKSS

SEQ ID NO: 1169 - protein, 15 aa, synthetic construct
RRTLKAIEITLKLLS

SEQ ID NO: 1170 - protein, 15 aa, synthetic construct
SKLEEALRWVTKVRS

SEQ ID NO: 1171 - protein, 20 aa, synthetic construct
KKLLDAADAVKKMLEKEKSS

SEQ ID NO: 1172 - protein, 16 aa, synthetic construct
KKLADAADWVETVKSS

SEQ ID NO: 1173 - protein, 17 aa, synthetic construct
AKLTKATKYALTVIKQS

SEQ ID NO: 1174 - protein, 20 aa, synthetic construct
KKVLETIRWIETVISRQRSS

SEQ ID NO: 1175 - protein, 16 aa, synthetic construct
KKTHSAIEWVERLVSS

SEQ ID NO: 1176 - protein, 24 aa, synthetic construct
RTLKDTTELTKNLNKKLKKLEEEL

SEQ ID NO: 1177 - protein, 21 aa, synthetic construct
ADLKKVAELVKKLMEEAKKKS

SEQ ID NO: 1178 - protein, 17 aa, synthetic construct
ALLLEAAKYVKKAREKS

SEQ ID NO: 1179 - protein, 30 aa, synthetic construct
RSNKKTKNKVKSIEKQVKEIEKRLEKLERA

SEQ ID NO: 1180 - protein, 18 aa, synthetic construct
TDTMKAARIMKEELKEKS

SEQ ID NO: 1181 - protein, 16 aa, synthetic construct
ADTKKAAEIAKKLAKS

SEQ ID NO: 1182 - protein, 24 aa, synthetic construct
RQIVEVMKEVEELRKRVENIEKNL

SEQ ID NO: 1183 - protein, 17 aa, synthetic construct
AKNAEAAKIAEETKRKD

SEQ ID NO: 1184 - protein, 17 aa, synthetic construct
RKLLEAAEEMEKMLKTS

SEQ ID NO: 1185 - protein, 28 aa, synthetic construct
QKTRATEEALKKTQKEVTKLKKEIQKLT

SEQ ID NO: 1186 - protein, 17 aa, synthetic construct
KKLKSAADDVKKAKEKS

SEQ ID NO: 1187 - protein, 17 aa, synthetic construct
RKMLEAVEHAKKLKKES

SEQ ID NO: 1188 - protein, 30 aa, synthetic construct
RSNKKTKNKVKSIEKQVKEIEKRLEKLEKA

SEQ ID NO: 1189 - protein, 17 aa, synthetic construct
KELKSAAEDVKKAKEKS

SEQ ID NO: 1190 - protein, 17 aa, synthetic construct
RKMLEAVEKAKKLDKES

SEQ ID NO: 1191 - protein, 18 aa, synthetic construct
REIIRAINIVRKIASEKS

SEQ ID NO: 1192 - protein, 19 aa, synthetic construct
RETKKATENVKTMLTKSKS

SEQ ID NO: 1193 - protein, 21 aa, synthetic construct
RKLEEIARIVEQKKRTEEKRS

SEQ ID NO: 1194 - protein, 18 aa, synthetic construct
RDLDTAAKQVKEMLKEKS

SEQ ID NO: 1195 - protein, 18 aa, synthetic construct
LELKKAAKAANTDLTKKS

SEQ ID NO: 1196 - protein, 13 aa, synthetic construct
RDLKKAAEIAKKS

SEQ ID NO: 1197 - protein, 18 aa, synthetic construct
RETEKTIRQVQEILKKWS

SEQ ID NO: 1198 - protein, 18 aa, synthetic construct
LELKEAAKAANTDLTKKS

SEQ ID NO: 1199 - protein, 19 aa, synthetic construct
RKTLETIEWVKKVIKKQRS

SEQ ID NO: 1200 - protein, 18 aa, synthetic construct
RDTIKVAIIVKELYKKIS

SEQ ID NO: 1201 - protein, 18 aa, synthetic construct
QKTISDLLEIVEKLIRSL

SEQ ID NO: 1202 - protein, 18 aa, synthetic construct
QKTISDLLEIIEKLIRSL

SEQ ID NO: 1203 - protein, 18 aa, synthetic construct
QKTISDLLEIVEQLIRSL

SEQ ID NO: 1204 - protein, 18 aa, synthetic construct
QKTISDLLEIVENLIRSL

SEQ ID NO: 1205 - protein, 18 aa, synthetic construct
QKTISDLLEIIESLLRSL

SEQ ID NO: 1206 - protein, 18 aa, synthetic construct
QETIQELLKIVKELIQKL

SEQ ID NO: 1207 - protein, 18 aa, synthetic construct
KETIKELLKIIKELIKEL

SEQ ID NO: 1208 - protein, 18 aa, synthetic construct
SQTISELLQIVKELLSQL

SEQ ID NO: 1209 - protein, 18 aa, synthetic construct
NKTIKELLNIIKSLLEKL

SEQ ID NO: 1210 - protein, 23 aa, synthetic construct
VATKKDLEDLIEKLERLLQKLDS

SEQ ID NO: 1211 - protein, 23 aa, synthetic construct
VATKKDLEDLIENLERLLQKLDS

SEQ ID NO: 1212 - protein, 23 aa, synthetic construct
VTTKKDLEDLIENLKRLLQKLDS

SEQ ID NO: 1213 - protein, 23 aa, synthetic construct
VTTKKDLEDLIENLERLLQKLDS

SEQ ID NO: 1214 - protein, 23 aa, synthetic construct
VATKKDLEDLIESLKRLLQKLDS

SEQ ID NO: 1215 - protein, 23 aa, synthetic construct
VATKKDLEDLIESLERLLQKLDS

SEQ ID NO: 1216 - protein, 23 aa, synthetic construct
VTTKKDLEDLIESLKRLLQKLDS

SEQ ID NO: 1217 - protein, 23 aa, synthetic construct
VTTKKDLEDLIESLERLLQKLDS

SEQ ID NO: 1218 - protein, 23 aa, synthetic construct
VATNKSLQDLIKELKDLLSKLNT

SEQ ID NO: 1219 - protein, 23 aa, synthetic construct
VTTKKELKDLIQKLKDLLSKLQT

SEQ ID NO: 1220 - protein, 23 aa, synthetic construct
VATKKELKDLITKLEKLLSKLQT

SEQ ID NO: 1221 - protein, 23 aa, synthetic construct
VTTKKELKDLIQKLEKLLSKLQT

SEQ ID NO: 1222 - protein, 22 aa, synthetic construct
NKVKKDVEELKESVRRLEKKLD

SEQ ID NO: 1223 - protein, 22 aa, synthetic construct
NKVKKDVEELKETVRRLEKKLD

SEQ ID NO: 1224 - protein, 22 aa, synthetic construct
NKVKKDVEELKENVRRLEKKLD

SEQ ID NO: 1225 - protein, 22 aa, synthetic construct
NKVKKDVEELKEQVRRLEKKLD

SEQ ID NO: 1226 - protein, 22 aa, synthetic construct
NKVKKDVEELKEEVRRLEKKLD

SEQ ID NO: 1227 - protein, 22 aa, synthetic construct
NKVKKDVEELKEDVRRLEKKLD

SEQ ID NO: 1228 - protein, 22 aa, synthetic construct
NKVKKDVEELKERVRRLEKKLD

SEQ ID NO: 1229 - protein, 22 aa, synthetic construct
NKVKKDVEELKEKVRRLEKKLD

SEQ ID NO: 1230 - protein, 22 aa, synthetic construct
NKVKKDVEELKEHVRRLEKKLD

SEQ ID NO: 1231 - protein, 22 aa, synthetic construct
NKVKKEVQELKQTVKSLEKELT

SEQ ID NO: 1232 - protein, 22 aa, synthetic construct
NKVKKDVNELKQSVKSLEKELT

SEQ ID NO: 1233 - protein, 22 aa, synthetic construct
NKVKKEVSELTEKVESLEKKLT

SEQ ID NO: 1234 - protein, 22 aa, synthetic construct
NKVKKDVTELSEKVESLEKKLT

SEQ ID NO: 1235 - protein, 27 aa, synthetic construct
GYIPEAPRDGQAYVRKDGEWVLLSTFL

SEQ ID NO: 1236 - protein, 488 aa, synthetic construct; identical to SEQ ID NO: 5

SEQ ID NO: 1237 - protein, 488 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVSKVLHLEGEVNKIKNALLSTNKAVVSLSNGVSVLTSKVLDLKNYINNQLLPIVNQQSCRIFNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELL

SEQ ID NO: 1238 - protein, 488 aa, synthetic construct
QNITEEFYQSTCSAVSRGYFSALRTGWYTSVITIELSNITETKCNGTDTKVKLIKQELDKYKNAVTELQLLMQNTPAANNRARREAPQHMNYTINTTKNLNVSISKKRKRRFLGFLLGVGSAIASGIAVCKVLHLEGEVNKIKNALLSTNKAVVSLSNGVSVLTFKVLDLKNYINNQLLPILNQQSCRIFNIETVIEFQQKNSRLLEITREFSVNAGVTTPLSTYMLTNSELLSLINDMPITNDQKKLMSSNVQIVRQQSYSIMCIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRKSDELL

SEQ ID NO: 1239 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 1240 - protein, 574 aa, synthetic construct
MELLIHRSSAIFLTLAVNALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 1241 - protein, 544 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 1242 - protein, 574 aa, synthetic construct; identical to SEQ ID NO: 1

(19) United States
(12) Patent Application Publication
(10) Pub. No.: US 2025/0188131 A1
(43) Pub. Date: Jun. 12, 2025

(54) VIRAL PROTEINS AND NANOSTRUCTURES AND USES THEREOF

(71) Applicant: Icosavax, Inc., Seattle, WA (US)

(72) Inventors: Quinton DOWLING, Seattle, WA (US); Daniel ELLIS, Seattle, WA (US)

(21) Appl. No.: 18/885,344

(22) Filed: Sep. 13, 2024

Related U.S. Application Data
(60) Provisional application No. 63/583,117, filed on Sep. 15, 2023.

(51) Int. Cl. C07K 14/005; A61K 39/00; A61P 37/04

(52) U.S. Cl. CPC C07K 14/005; A61P 37/04; A61K 39/00; A61K 2039/70; C12N 2760/18522; C12N 2760/18534; C12N 2760/18571

(57) ABSTRACT

Provided herein are recombinant polypeptides comprising an engineered ectodomain of a viral protein from enveloped viruses. Also provided herein are two-component protein nanostructures and compositions for use in vaccinating, generating an immune response, or treating or preventing a viral infection.

### CROSS-REFERENCE TO RELATED APPLICATION

This application claims the benefit of U.S. Provisional Application No. 63/583,117, filed Sep. 15, 2023, the contents of which is incorporated by reference herein in its entirety.

### INCORPORATION BY REFERENCE OF SEQUENCE LISTING

The instant application contains a Sequence Listing which has been submitted electronically in XML format and is hereby incorporated by reference in its entirety. Said XML copy, created on Sep. 11, 2024, is named 061291-518001WO.xml and is 1,130 KB in size.

### BACKGROUND

When an enveloped virus encounters a target cell, its viral membrane fusion protein undergoes a conformational change that drives fusion of the viral envelope with the target cell's cell membrane. This fusion process delivers the viral genome into the target cell. For many enveloped viruses, the adaptive immune response to the viral membrane fusion protein is a key source of protective immunity, in part because neutralizing antibodies may inhibit this fusion process. Hence, vaccines for enveloped viruses often include a viral membrane fusion protein as an antigen.

There is an unmet need for viral membrane fusion proteins stabilized by designed amino acid substitutions. The present disclosure provides recombinant polypeptides and related compositions and methods that address this need for Respiratory Syncytial Virus (RSV), hMPV, PIV3, PIV5, SARS-COV-2, and Nipah virus.

### SUMMARY

In one aspect, the disclosure provides a recombinant polypeptide, comprising an engineered ectodomain of a trimeric pathogenic (e.g., viral) protein, wherein the ectodomain comprises a C-terminal helix-forming segment comprising one or more amino acid substitutions, relative to a native reference sequence of the pathogenic (e.g., viral) protein, selected such that the segment forms a stable alpha-helical homotrimer. In another aspect, the disclosure provides a nanostructure comprising a trimeric component comprising a helix-forming segment as disclosed herein. In another aspect, the disclosure provides helix-forming segments as disclosed herein.

In some embodiments of the recombinant polypeptide, the C-terminal helix forming segment has improved hydrophobic packing compared to the native reference sequence. In some embodiments, the C-terminal helix forming segment comprises between about 7 and about 31 residues. In some embodiments, the amino acid substitutions comprise polar, charged and/or hydrophobic amino acids. In some embodiments, the C-terminal helix forming segment comprises a polypeptide sequence according to any one of LXXTIXXLLXIXXXLXXXL (SEQ ID NO: 566), LVXTXKXLXDLIXXLXXLLXKLXX (SEQ ID NO: 567), LNKVKKXVXXLXXXVXXLEKXLX (SEQ ID NO: 568), EKIXXAIKKAXKL (SEQ ID NO: 569), EXIXKAIKXLXXXXX (SEQ ID NO: 570), XKXXEXXXXVXXXXXXXXX (SEQ ID NO: 571), XXLKKAAXIXKKXLKXX (SEQ ID NO: 572).

In some embodiments, the C-terminal helix forming segment comprises a polypeptide sequence according to any one of the consensus sequences in Table 24.

In some embodiments, the segment comprises a polypeptide sequence according to any one of L X₂ X₂ T I X₂ X₂ L L X₂ I [V/I] X₂ X₂ L [I/L] X₂ X₂ L (SEQ ID NO: 573), L V [A/T] T X₂ K X₂ L X₂ D L I X₂ X₂ L [K/E] X₂ L L X₂ K L X₂ X₂ (SEQ ID NO: 574), or L N K V K K X₂ V X₂ X₂ L X₂ X₂ X₂ V X₂ X₂ L E K X₂ L X₂ (SEQ ID NO: 575), wherein X₂ is polar and charged residues selected from S, T, N, Q, E, D, R, K, and H, preferably wild type amino acid.

In some embodiments, segment comprises a polypeptide sequence according any one of E K I X₂ X₂ A I K K A X₂ K L (SEQ ID NO: 576), E X₂ I X₂ K A I K X₂ L [L/X₂] X₂ X₂ [X₁/X₂] X₂ (SEQ ID NO: 577), X₂ K [X₁/T] [L/E] E [T/A] X₁ X₂ [I/X₂] V X₂ X₂ [X₁/X₂] [X₁/X₂] X₂ X₂ X₁ X₂ X₂ (SEQ ID NO: 578), or X₂ X₂ L K K A A X₂ I X₁ K K X₁ L K X₂ X₂ (SEQ ID NO: 579), wherein X₁ is apolar residues selected from A, I, L, and M, and wherein X₂ is polar and charged residues selected from S, T, N, Q, E, D, R, K, and H, preferably wild type amino acid. In some embodiments, the segment comprises a polypeptide sequence listed in Table 25A or Table 25B. In some embodiments, the native reference sequence of the viral protein is any one of SEQ ID NOs: 1, 104, 327, 382, 459, 499.

In some embodiments, the recombinant polypeptide comprises an engineered ectodomain of an hMPV fusion (F) protein, wherein the ectodomain comprises a C-terminal helix-forming segment, between about residue 470 and about residue 500 relative to SEQ ID NO: 104, comprising one or more amino acid substitutions selected such that the segment forms a stable alpha-helical homotrimer. In some embodiments, the C-terminal helix-forming segment comprises substitutions relative to the reference sequence SEQ ID NO: 104 at two or more, three or more, or four or more residues that generate hydrophobic contacts between the segments in the alpha-helical homotrimer. In some embodiments, the ectodomain comprises the C-terminal helix-forming segment, between about residue 470 and about residue 490 relative to SEQ ID NO: 104, comprising one or more amino acid substitutions selected such that the segment forms a stable alpha-helical homotrimer. In some embodiments, the C-terminal helix-forming segment comprises between about 7 and about 21 residues.

In some embodiments, the segment comprises (1) an amino acid substitution at position Q471 relative to SEQ ID NO: 104, wherein Q is substituted with any one of A, D, E, I, Q, R, S, T, (2) an amino acid substitution at position A472 relative to SEQ ID NO: 104, wherein A is substituted with any one of A, D, E, I, K, R, S, T, Y, (3) an amino acid substitution at position L473 relative to SEQ ID NO: 104, wherein L is substituted with any one of A, I, L, M, Q, S, T, W, (4) an amino acid substitution at position V474 relative to SEQ ID NO: 104, wherein V is substituted with any one of A, D, E, I, K, L, N, Q, S, T, (5) an amino acid substitution at position D475 relative to SEQ ID NO: 104, wherein D is substituted with any one of A, D, E, H, K, N, Q, R, S, T, (6) an amino acid substitution at position Q476 relative to SEQ ID NO: 104, wherein Q is substituted with any one of A, D, E, H, I, K, L, M, N, Q, T, V, (7) an amino acid substitution at position S477 relative to SEQ ID NO: 104, wherein S is substituted with any one of A, E, I, K, L, M, N, Q, R, S, T, V, (8) an amino acid substitution at position N478 relative to SEQ ID NO: 104, wherein N is substituted with any one of A, D, E, K, N, Q, R, S, T, (9) an amino acid substitution at position R479 relative to SEQ ID NO: 104, wherein R is substituted with any one of A, D, E, F, I, K, L, M, N, Q, R, S, T, W, Y, (10) an amino acid substitution at position I480 relative to SEQ ID NO: 104, wherein I is substituted with any one of A, I, L, M, R, S, T, V, (11) an amino acid substitution at position L481 relative to SEQ ID NO: 104, wherein L is substituted with any one of D, E, I, K, L, M, N, Q, R, S, T, (12) an amino acid substitution at position S482 relative to SEQ ID NO: 104, wherein S is substituted with any one of A, D, E, K, Q, R, S, T, (13) an amino acid substitution at position S483 relative to SEQ ID NO: 104, wherein S is substituted with any one of A, D, E, F, H, I, K, L, M, N, Q, R, S, T, V, W, Y, (14) an amino acid substitution at position A484 relative to SEQ ID NO: 104, wherein A is substituted with any one of A, D, E, I, K, L, M, R, S, T, V, Y, (15) an amino acid substitution at position E485 relative to SEQ ID NO: 104, wherein E is substituted with any one of D, E, G, K, L, Q, R, S, T, (16) an amino acid substitution at position K486 relative to SEQ ID NO: 104, wherein K is substituted with any one of A, E, I, K, L, Q, R, S, T, (17) an amino acid substitution at position G487 relative to SEQ ID NO: 104, wherein G is substituted with any one of A, E, I, K, L, R, S, T, V, (18) an amino acid substitution at position N488 relative to SEQ ID NO: 104, wherein N is substituted with any one of E, I, K, L, N, Q, R, S, (19) an amino acid substitution at position T489 relative to SEQ ID NO: 104, wherein T is substituted with any one of A, D, E, K, S, and/or (20) any combination of (1)-(19). In some embodiments, the segment comprises a polypeptide sequence listed in Table 6B, or a polypeptide sequence having between 1 and 5 amino acid substitutions thereto.

In some embodiments, the ectodomain comprises the C-terminal helix-forming segment, between about residue 470 and about residue 500 relative to SEQ ID NO: 104, comprising one or more amino acid substitutions selected such that the segment forms a stable alpha-helical homotrimer. In some embodiments, the C-terminal helix-forming segment comprises between about 16 and about 30 residues.

In some embodiments, the segment comprises (1) an amino acid substitution at position A472 relative to SEQ ID NO: 104, wherein A is substituted with any one of T, N, K, R, E, S, (2) an amino acid substitution at position L473 relative to SEQ ID NO: 104, wherein L is substituted with any one of T, I, V, (3) an amino acid substitution at position V474 relative to SEQ ID NO: 104, wherein V is substituted with any one of E, Q, L, D, (4) an amino acid substitution at position D475 relative to SEQ ID NO: 104, wherein D is substituted with any one of E, D, K, (5) an amino acid substitution at position Q476 relative to SEQ ID NO: 104, wherein Q is substituted with any one of Q, R, D, A, T, S, K, (6) an amino acid substitution at position S477 relative to SEQ ID NO: 104, wherein S is substituted with any one of I, V, L, (7) an amino acid substitution at position N478 relative to SEQ ID NO: 104, wherein N is substituted with any one of K, E, N, S, (8) an amino acid substitution at position R479 relative to SEQ ID NO: 104, wherein R is substituted with any one of T, D, E, S, Y, A, (9) an amino acid substitution at position I480 relative to SEQ ID NO: 104, wherein I is substituted with any one of L, N, (10) an amino acid substitution at position L481 relative to SEQ ID NO: 104, wherein L is substituted with any one of T, D, E, K, Q, S, N, (11) an amino acid substitution at position S482 relative to SEQ ID NO: 104, wherein S is substituted with any one of E, D, S, T, Q, K, (12) an amino acid substitution at position S483 relative to SEQ ID NO: 104, wherein S is substituted with any one of R, K, L, E, A, (13) an amino acid substitution at position A484 relative to SEQ ID NO: 104, wherein A is substituted with any one of V, I, M, (14) an amino acid substitution at position E485 relative to SEQ ID NO: 104, wherein E is substituted with any one of E, A, K, H, Q, S, N, (15) an amino acid substitution at position K486 relative to SEQ ID NO: 104, wherein K is substituted with any one of S, E, K, R, V, D, H, (16) an amino acid substitution at position G487 relative to SEQ ID NO: 104, wherein G is substituted with any one of I, L, (17) an amino acid substitution at position N488 relative to SEQ ID NO: 104, wherein N is substituted with any one of E, K, R, (18) an amino acid substitution at position T489 relative to SEQ ID NO: 104, wherein T is substituted with any one of K, E, S, R, Q, (19) an amino acid substitution at position S490 relative to SEQ ID NO: 104, wherein S is substituted with any one of E, V, T, R, L, (20) an amino acid substitution at position G491 relative to SEQ ID NO: 104, wherein G is substituted with any one of GL, I, V, (21) an amino acid substitution at position R492 relative to SEQ ID NO: 104, wherein R is substituted with any one of E, Q, S, A, D, (22) an amino acid substitution at position E493 relative to SEQ ID NO: 104, wherein E is substituted with any one of A, E, N, L, K, Q, S, (23) an amino acid substitution at position N494 relative to SEQ ID NO: 104, wherein N is substituted with any one of I, L, (24) an amino acid substitution at position L495 relative to SEQ ID NO: 104, wherein L is substituted with any one of K, L, T, V, I, (25) an amino acid substitution at position Y496 relative to SEQ ID NO: 104, wherein Y is substituted with any one of K, E, R, Q, (26) an amino acid substitution at position F497 relative to SEQ ID NO: 104, wherein F is substituted with any one of D, R, E, Q, (27) an amino acid substitution at position Q498 relative to SEQ ID NO: 104, wherein Q is substituted with any one of V, L, and/or (28) any combination of (1)-(27). In some embodiments, the segment comprises a polypeptide sequence listed in Table 6D, or a polypeptide sequence having between 1 and 5 amino acid substitutions thereto.

In some embodiments, the ectodomain further comprises one, two, three or more amino acid substitutions at positions 63, 97, 98, 99, 100, 101, 102, 140, 147, 153, 185, 188, 219, 231, 294, 365, 368, 450, 463, or 470 relative to SEQ ID NO: 104.

In some embodiments, the recombinant polypeptide comprises an engineered ectodomain of a PIV3 fusion (F) protein, wherein the ectodomain comprises a C-terminal helix-forming segment, between about residue 460 and about residue 490 relative to SEQ ID NO: 327, comprising one or more amino acid substitutions selected such that the segment forms a stable alpha-helical homotrimer. In some embodiments, the C-terminal helix-forming segment comprises substitutions relative to the reference sequence SEQ ID NO: 327 at two or more, three or more, or four or more residues that generate hydrophobic contacts between the segments in the alpha-helical homotrimer. In some embodiments, the ectodomain comprises the C-terminal helix-forming segment, between about residue 460 and about residue 480 relative to SEQ ID NO: 104, comprising one or more amino acid substitutions selected such that the segment forms a stable alpha-helical homotrimer. In some embodiments, the C-terminal helix-forming segment comprises between about 20 and about 28 residues.

In some embodiments, the segment comprises (1) an amino acid substitution at position L460 relative to SEQ ID NO: 327, wherein L is substituted with any one of L, M, V, (2) an amino acid substitution at position N461 relative to SEQ ID NO: 327, wherein N is substituted with N, (3) an amino acid substitution at position K462 relative to SEQ ID NO: 327, wherein K is substituted with any one of K, R, (4) an amino acid substitution at position V463 relative to SEQ ID NO: 327, wherein V is substituted with any one of L, V, T, (5) an amino acid substitution at position K464 relative to SEQ ID NO: 327, wherein K is substituted with any one of A, K, Q, (6) an amino acid substitution at position S465 relative to SEQ ID NO: 327, wherein S is substituted with any one of K, S, (7) an amino acid substitution at position D466 relative to SEQ ID NO: 327, wherein D is substituted with any one of E, K, (8) an amino acid substitution at position L467 relative to SEQ ID NO: 327, wherein L is substituted with any one of V, L, T, (9) an amino acid substitution at position E468 relative to SEQ ID NO: 327, wherein E is substituted with any one of K, D, E, (10) an amino acid substitution at position E469 relative to SEQ ID NO: 327, wherein E is substituted with any one of T, Q, K, E, (11) an amino acid substitution at position S470 relative to SEQ ID NO: 327, wherein S is substituted with any one of I, L, M, Y, F, W, (12) an amino acid substitution at position K471 relative to SEQ ID NO: 327, wherein K is substituted with any one of L, W, A, I, (13) an amino acid substitution at position E472 relative to SEQ ID NO: 327, wherein E is substituted with any one of K, E, (14) an amino acid substitution at position W473 relative to SEQ ID NO: 327, wherein W is substituted with any one of E, I, K, Q, (15) an amino acid substitution at position Y474 relative to SEQ ID NO: 327, wherein Y is substituted with any one of L, M, T, V, E, (16) an amino acid substitution at position R475 relative to SEQ ID NO: 327, wherein R is substituted with any one of S, K, R, A, (17) an amino acid substitution at position R476 relative to SEQ ID NO: 327, wherein R is substituted with any one of K, E, S, N, (18) an amino acid substitution at position S477 relative to SEQ ID NO: 327, wherein S is substituted with any one of K, D, E, and/or (19) any combination of (1)-(18).

In some embodiments, the segment comprises a polypeptide sequence listed in Table 7B, or a polypeptide sequence having between 1 and 5 amino acid substitutions thereto. In some embodiments, the ectodomain comprises the C-terminal helix-forming segment, between about residue 465 and about residue 490 relative to SEQ ID NO: 327, comprising one or more amino acid substitutions selected such that the segment forms a stable alpha-helical homotrimer. In some embodiments, the C-terminal helix-forming segment comprises between about 14 and about 30 residues.

In some embodiments, the segment comprises (1) an amino acid substitution at position S465 relative to SEQ ID NO: 327, wherein S is substituted with any one of E, K, D, S, N, Q, T, R, A, (2) an amino acid substitution at position D466 relative to SEQ ID NO: 327, wherein D is substituted with any one of D, R, K, E, M, Q, A, S, N, (3) an amino acid substitution at position L467 relative to SEQ ID NO: 327, wherein L is substituted with any one of I, V, L, (4) an amino acid substitution at position E468 relative to SEQ ID NO: 327, wherein E is substituted with any one of E, K, S, D, R, H, T, N, A, (5) an amino acid substitution at position E469 relative to SEQ ID NO: 327, wherein E is substituted with any one of K, S, E, N, T, Q, H, D, Y, (6) an amino acid substitution at position S470 relative to SEQ ID NO: 327, wherein S is substituted with any one of L, D, V, I, A, N, T, (7) an amino acid substitution at position K471 relative to SEQ ID NO: 327, wherein K is substituted with any one of E, T, L, K, N, I, R, Q, S, (8) an amino acid substitution at position E472 relative to SEQ ID NO: 327, wherein E is substituted with any one of E, K, Q, S, H, R, T, (9) an amino acid substitution at position W473 relative to SEQ ID NO: 327, wherein W is substituted with any one of R, Q, K, E, T, S, I, N, (10) an amino acid substitution at position Y474 relative to SEQ ID NO: 327, wherein Y is substituted with any one of V, L, I, Q, T, (11) an amino acid substitution at position R475 relative to SEQ ID NO: 327, wherein R is substituted with any one of H, K, D, T, E, S, R, N, Q, A, (12) an amino acid substitution at position R476 relative to SEQ ID NO: 327, wherein R is substituted with any one of A, T, H, E, D, K, R, Q, S, (13) an amino acid substitution at position S477 relative to SEQ ID NO: 327, wherein S is substituted with any one of I, L, V, (14) an amino acid substitution at position N478 relative to SEQ ID NO: 327, wherein N is substituted with any one of E, L, K, I, R, S, Q, (15) an amino acid substitution at position Q479 relative to SEQ ID NO: 327, wherein Q is substituted with any one of K, H, E, N, Q, R, T, A, S, (16) an amino acid substitution at position K480 relative to SEQ ID NO: 327, wherein K is substituted with any one of K, R, E, T, S, L, A, I, V, (17) an amino acid substitution at position L481 relative to SEQ ID NO: 327, wherein L is substituted with any one of L, V, I, (18) an amino acid substitution at position D482 relative to SEQ ID NO: 327, wherein D is substituted with any one of K, A, E, S, H, T, N, D, R, (19) an amino acid substitution at position S483 relative to SEQ ID NO: 327, wherein S is substituted with any one of Q, T, E, A, S, N, D, K, L, (20) an amino acid substitution at position I484 relative to SEQ ID NO: 327, wherein I is substituted with any one of I, L, A, V, (21) an amino acid substitution at position G485 relative to SEQ ID NO: 327, wherein G is substituted with any one of L, K, R, E, I, (22) an amino acid substitution at position S486 relative to SEQ ID NO: 327, wherein S is substituted with any one of T, A, E, R, H, D, S, and/or (23) any combination of (1)-(22). In some embodiments, the segment comprises a polypeptide sequence listed in Table 7D, or a polypeptide sequence having between 1 and 5 amino acid substitutions thereto.

In some embodiments, the recombinant polypeptide comprises an engineered ectodomain of a PIV5 fusion (F) protein, wherein the ectodomain comprises a C-terminal helix-forming segment, between about residue 460 and about residue 490 relative to SEQ ID NO: 382, comprising one or more amino acid substitutions selected such that the segment forms a stable alpha-helical homotrimer. In some embodiments, the C-terminal helix-forming segment comprises substitutions relative to the reference sequence SEQ ID NO: 382 at two or more, three or more, or four or more residues that generate hydrophobic contacts between the segments in the alpha-helical homotrimer. In some embodiments, the ectodomain comprises the C-terminal helix-forming segment, between about residue 460 and about residue 480 relative to SEQ ID NO: 104, comprising one or more amino acid substitutions selected such that the segment forms a stable alpha-helical homotrimer. In some embodiments, the C-terminal helix-forming segment comprises between about 6 and about 26 residues.

In some embodiments, the segment comprises (1) an amino acid substitution at position A463 relative to SEQ ID NO: 382, wherein A is substituted with any one of L, T, V, A, (2) an amino acid substitution at position L464 relative to SEQ ID NO: 382, wherein L is substituted with any one of K, I, Q, A, W, E, (3) an amino acid substitution at position Q465 relative to SEQ ID NO: 382, wherein Q is substituted with any one of K, Q, T, E, S, R, (4) an amino acid substitution at position H466 relative to SEQ ID NO: 382, wherein H is substituted with any one of K, A, E, L, I, W, R, Q, T, D, Y, (5) an amino acid substitution at position L467 relative to SEQ ID NO: 382, wherein Lis substituted with any one of V, I, L, M, FA, T, C, H, (6) an amino acid substitution at position A468 relative to SEQ ID NO: 382, wherein A is substituted with any one of D, T, K, L, E, R, I, N, S, (7) an amino acid substitution at position Q469 relative to SEQ ID NO: 382, wherein Q is substituted with any one of E, K, S, T, A, R, Q, D, (8) an amino acid substitution at position S470 relative to SEQ ID NO: 382, wherein S is substituted with any one of A, K, L, I, T, S, V, H, Y, E, W, FR, Q, M, (9) an amino acid substitution at position D471 relative to SEQ ID NO: 382, wherein D is substituted with any one of T, E, V, L, S, I, A, K, Y, W, (10) an amino acid substitution at position T472 relative to SEQ ID NO: 382, wherein T is substituted with any one of K, E, R, S, T, A, D, L, (11) an amino acid substitution at position Y473 relative to SEQ ID NO: 382, wherein Y is substituted with any one of T, K, S, R, Q, D, E, I, H, M, (12) an amino acid substitution at position L474 relative to SEQ ID NO: 382, wherein L is substituted with any one of T, S, L, A, D, W, Q, I, Y, V, K, E, (13) an amino acid substitution at position S475 relative to SEQ ID NO: 382, wherein S is substituted with any one of T, E, I, K, S, Q, A, L, R, D, (14) an amino acid substitution at position A476 relative to SEQ ID NO: 382, wherein A is substituted with any one of R, K, A, S, E, I, T, D, Q, (15) an amino acid substitution at position I477 relative to SEQ ID NO: 382, wherein I is substituted with any one of K, Q, R, D, T, E, I, Y, S, L, (16) an amino acid substitution at position T478 relative to SEQ ID NO: 382, wherein Tis substituted with any one of E, K, S, D, W, L, Q, I, T, (17) an amino acid substitution at position S479 relative to SEQ ID NO: 382, wherein S is substituted with any one of R, K, Q, S, A, D, E, (18) an amino acid substitution at position A480 relative to SEQ ID NO: 382, wherein A is substituted with any one of S, K, (19) an amino acid substitution at position T481 relative to SEQ ID NO: 382, wherein T is substituted with any one of E, D, S, K, M, N, A, T, (20) an amino acid substitution at position T482 relative to SEQ ID NO: 382, wherein T is substituted with any one of R, S, Q, L, K, (21) an amino acid substitution at position T483 relative to SEQ ID NO: 382, wherein Tis substituted with any one of K, A, S, (22) an amino acid substitution at position S484 relative to SEQ ID NO: 382, wherein S is substituted with any one of S, E, D, Y, (23) an amino acid substitution at position V485 relative to SEQ ID NO: 382, wherein V is substituted with any one of Q, T, (24) an amino acid substitution at position L486 relative to SEQ ID NO: 382, wherein L is substituted with K, (25) an amino acid substitution at position S487 relative to SEQ ID NO: 382, wherein S is substituted with any one of S, K, (26) an amino acid substitution at position I488 relative to SEQ ID NO: 382, wherein I is substituted with any one of S, K, and/or (27) any combination of (1)-(26).

In some embodiments, the segment comprises a polypeptide sequence listed in Table 8B, or a polypeptide sequence having between 1 and 5 amino acid substitutions thereto.

In some embodiments, the polypeptides comprises an engineered ectodomain of a SARS-CoV2 spike(S) protein, wherein the ectodomain comprises a C-terminal helix-forming segment, between about residue 1140 and about residue 1170 relative to SEQ ID NO: 459, comprising one or more amino acid substitutions selected such that the segment forms a stable alpha-helical homotrimer. In some embodiments, the C-terminal helix-forming segment comprises substitutions relative to the reference sequence SEQ ID NO: 459 at two or more, three or more, or four or more residues that generate hydrophobic contacts between the segments in the alpha-helical homotrimer. In some embodiments, the ectodomain comprises the C-terminal helix-forming segment, between about residue 1140 and about residue 1170 relative to SEQ ID NO: 459, comprising one or more amino acid substitutions selected such that the segment forms a stable alpha-helical homotrimer. In some embodiments, the C-terminal helix-forming segment comprises between about 10 and about 25 residues.

In some embodiments, the segment comprises (1) an amino acid substitution at position D1147 relative to SEQ ID NO: 459, wherein D is substituted with any one of E, D, K, (2) an amino acid substitution at position S1148 relative to SEQ ID NO: 459, wherein S is substituted with any one of T, S, K, (3) an amino acid substitution at position F1149 relative to SEQ ID NO: 459, wherein F is substituted with A, (4) an amino acid substitution at position K1150 relative to SEQ ID NO: 459, wherein K is substituted with any one of I, A, L, M, (5) an amino acid substitution at position E1151 relative to SEQ ID NO: 459, wherein E is substituted with any one of K, S, D, R, E, (6) an amino acid substitution at position E1152 relative to SEQ ID NO: 459, wherein E is substituted with any one of I, Y, K, T, R, E, (7) an amino acid substitution at position L1153 relative to SEQ ID NO: 459, wherein L is substituted with any one of T, A, (8) an amino acid substitution at position D1154 relative to SEQ ID NO: 459, wherein D is substituted with any one of L, I, E, T, M, V, (9) an amino acid substitution at position K1155 relative to SEQ ID NO: 459, wherein K is substituted with any one of E, K, T, R, (10) an amino acid substitution at position Y1156 relative to SEQ ID NO: 459, wherein Y is substituted with any one of I, V, K, R, (11) an amino acid substitution at position F1157 relative to SEQ ID NO: 459, wherein F is substituted with any one of V, A, I, Y, T, S, (12) an amino acid substitution at position K1158 relative to SEQ ID NO: 459, wherein K is substituted with any one of L, R, S, K, D, W, N, I, (13) an amino acid substitution at position N1159 relative to SEQ ID NO: 459, wherein N is substituted with any one of K, T, Q, I, R, E, (14) an amino acid substitution at position H1160 relative to SEQ ID NO: 459, wherein His substituted with any one of I, L, R, E, K, S, (15) an amino acid substitution at position T1161 relative to SEQ ID NO: 459, wherein T is substituted with any one of L, N, I, A, S, W, Y, (16) an amino acid substitution at position S1162 relative to SEQ ID NO: 459, wherein S is substituted with any one of K, S, T, R, (17) an amino acid substitution at position P1163 relative to SEQ ID NO: 459, wherein P is substituted with any one of E, D, R, K, I, A, (18) an amino acid substitution at position D1164 relative to SEQ ID NO: 459, wherein D is substituted with any one of W, S, M, D, T, I, N, (19) an amino acid substitution at position V1165 relative to SEQ ID NO: 459, wherein V is substituted with any one of E, A, K, L, (20) an amino acid substitution at position D1166 relative to SEQ ID NO: 459, wherein D is substituted with any one of K, S, (21) an amino acid substitution at position L1167 relative to SEQ ID NO: 459, wherein L is substituted with any one of R, K, (22) an amino acid substitution at position G1168 relative to SEQ ID NO: 459, wherein G is substituted with any one of K, S, (23) an amino acid substitution at position D1169 relative to SEQ ID NO: 459, wherein D is substituted with S, (24) an amino acid substitution at position I1170 relative to SEQ ID NO: 459, wherein I is substituted with S, and/or (25) any combination of (1)-(24).

In some embodiments, the segment comprises a polypeptide sequence listed in Table 9B, or a polypeptide sequence having between 1 and 5 amino acid substitutions thereto. In some embodiments, the ectodomain comprises the C-terminal helix-forming segment, between about residue 1145 and about residue 1175 relative to SEQ ID NO: 459, comprising one or more amino acid substitutions selected such that the segment forms a stable alpha-helical homotrimer. In some embodiments, the C-terminal helix-forming segment comprises between about 12 and about 22 residues.

In some embodiments, the segment comprises (1) an amino acid substitution at position D1147 relative to SEQ ID NO: 459, wherein D is substituted with any one of Q, T, E, S, N, D, K, (2) an amino acid substitution at position S1148 relative to SEQ ID NO: 459, wherein S is substituted with any one of T, K, N, R, S, A, E, (3) an amino acid substitution at position F1149 relative to SEQ ID NO: 459, wherein F is substituted with any one of L, T, I, V, (4) an amino acid substitution at position K1150 relative to SEQ ID NO: 459, wherein K is substituted with any one of K, Q, R, H, S, E, (5) an amino acid substitution at position E1151 relative to SEQ ID NO: 459, wherein E is substituted with any one of E, N, A, S, K, T, D, (6) an amino acid substitution at position E1152 relative to SEQ ID NO: 459, wherein E is substituted with any one of E, T, V, R, K, N, (7) an amino acid substitution at position L1153 relative to SEQ ID NO: 459, wherein L is substituted with any one of S, V, T, A, (8) an amino acid substitution at position D1154 relative to SEQ ID NO: 459, wherein D is substituted with any one of T, L, E, I, V, (9) an amino acid substitution at position K1155 relative to SEQ ID NO: 459, wherein K is substituted with any one of H, E, S, T, Q, (10) an amino acid substitution at position Y1156 relative to SEQ ID NO: 459, wherein Y is substituted with any one of L, E, I, T, A, S, R, (11) an amino acid substitution at position F1157 relative to SEQ ID NO: 459, wherein F is substituted with any one of T, V, I, A, S, M, (12) an amino acid substitution at position K1158 relative to SEQ ID NO: 459, wherein K is substituted with any one of K, E, N, R, T, A, Q, I, (13) an amino acid substitution at position N1159 relative to SEQ ID NO: 459, wherein N is substituted with any one of T, E, A, K, Q, (14) an amino acid substitution at position H1160 relative to SEQ ID NO: 459, wherein His substituted with any one of L, M, A, E, T, Y, I, S, (15) an amino acid substitution at position T1161 relative to SEQ ID NO: 459, wherein T is substituted with any one of L, I, (16) an amino acid substitution at position S1162 relative to SEQ ID NO: 459, wherein S is substituted with any one of S, R, K, N, E, Q, (17) an amino acid substitution at position P1163 relative to SEQ ID NO: 459, wherein P is substituted with any one of E, S, T, R, (18) an amino acid substitution at position D1164 relative to SEQ ID NO: 459, wherein D is substituted with any one of T, M, A, (19) an amino acid substitution at position V1165 relative to SEQ ID NO: 459, wherein V is substituted with any one of A, L, and/or (20) any combination of (1)-(19).

In some embodiments, the segment comprises a polypeptide sequence listed in Table 9D, or a polypeptide sequence having between 1 and 5 amino acid substitutions thereto.

In some embodiments, the recombinant polypeptide comprises an engineered ectodomain of a Nipah fusion (F) protein, wherein the ectodomain comprises a C-terminal helix-forming segment, between about residue 460 and about residue 490 relative to SEQ ID NO: 499, comprising one or more amino acid substitutions selected such that the segment forms a stable alpha-helical homotrimer. In some embodiments, the C-terminal helix-forming segment comprises substitutions relative to the reference sequence SEQ ID NO: 499 at two or more, three or more, or four or more residues that generate hydrophobic contacts between the segments in the alpha-helical homotrimer. In some embodiments, the ectodomain comprises the C-terminal helix-forming segment, between about residue 460 and about residue 490 relative to SEQ ID NO: 499, comprising one or more amino acid substitutions selected such that the segment forms a stable alpha-helical homotrimer. In some embodiments, the C-terminal helix-forming segment comprises between about 16 and about 33 residues.

In some embodiments, the segment comprises (1) an amino acid substitution at position M463 relative to SEQ ID NO: 499, wherein M is substituted with any one of I, A, T, L, M, (2) an amino acid substitution at position N464 relative to SEQ ID NO: 499, wherein N is substituted with N, (3) an amino acid substitution at position Q465 relative to SEQ ID NO: 499, wherein Q is substituted with any one of E, L, K, T, S, I, D, (4) an amino acid substitution at position S466 relative to SEQ ID NO: 499, wherein S is substituted with S, (5) an amino acid substitution at position L467 relative to SEQ ID NO: 499, wherein L is substituted with any one of M, L, I, V, T, (6) an amino acid substitution at position Q468 relative to SEQ ID NO: 499, wherein Q is substituted with any one of E, K, A, T, S, D, R, I, Q, (7) an amino acid substitution at position Q469 relative to SEQ ID NO: 499, wherein Q is substituted with any one of R, S, K, T, E, Q, (8) an amino acid substitution at position S470 relative to SEQ ID NO: 499, wherein S is substituted with any one of T, L, I, V, A, (9) an amino acid substitution at position K471 relative to SEQ ID NO: 499, wherein K is substituted with any one of K, A, W, E, L, I, (10) an amino acid substitution at position D472 relative to SEQ ID NO: 499, wherein D is substituted with any one of K, T, R, Q, E, (11) an amino acid substitution at position Y473 relative to SEQ ID NO: 499, wherein Y is substituted with any one of W, D, K, Y, I, M, E, T, (12) an amino acid substitution at position I474 relative to SEQ ID NO: 499, wherein I is substituted with any one of I, V, M, L, A, (13) an amino acid substitution at position K475 relative to SEQ ID NO: 499, wherein K is substituted with any one of T, K, M, R, E, L, A, S, (14) an amino acid substitution at position E476 relative to SEQ ID NO: 499, wherein E is substituted with any one of K, S, A, E, T, D, (15) an amino acid substitution at position A477 relative to SEQ ID NO: 499, wherein A is substituted with any one of L, I, V, FT, A, M, W, K, Y, (16) an amino acid substitution at position Q478 relative to SEQ ID NO: 499, wherein Q is substituted with any one of I, K, A, L, E, D, S, Y, (17) an amino acid substitution at position R479 relative to SEQ ID NO: 499, wherein R is substituted with any one of A, S, K, R, T, L, E, (18) an amino acid substitution at position L480 relative to SEQ ID NO: 499, wherein L is substituted with any one of K, E, R, Y, T, Q, (19) an amino acid substitution at position L481 relative to SEQ ID NO: 499, wherein L is substituted with any one of W, I, V, L, E, S, Q, A, T, (20) an amino acid substitution at position D482 relative to SEQ ID NO: 499, wherein D is substituted with any one of K, Q, E, W, T, S, A, (21) an amino acid substitution at position T483 relative to SEQ ID NO: 499, wherein T is substituted with any one of S, T, K, R, Q, (22) an amino acid substitution at position V484 relative to SEQ ID NO: 499, wherein V is substituted with any one of R, E, I, S, Y, L, K, D, (23) an amino acid substitution at position N485 relative to SEQ ID NO: 499, wherein N is substituted with any one of I, R, K, W, E, T, (24) an amino acid substitution at position P486 relative to SEQ ID NO: 499, wherein P is substituted with any one of A, T, R, K, Q, (25) an amino acid substitution at position S487 relative to SEQ ID NO: 499, wherein S is substituted with any one of K, R, T, S, (26) an amino acid substitution at position L488 relative to SEQ ID NO: 499, wherein L is substituted with any one of E, V, L, K, (27) an amino acid substitution at position I489 relative to SEQ ID NO: 499, wherein I is substituted with any one of E, Q, L, K, R, and/or (28) any combination of (1)-(27).

In some embodiments, the segment comprises a polypeptide sequence listed in Table 10B, or a polypeptide sequence having between 1 and 5 amino acid substitutions thereto. In some embodiments, the ectodomain comprises the C-terminal helix-forming segment, between about residue 460 and about residue 490 relative to SEQ ID NO: 499, comprising one or more amino acid substitutions selected such that the segment forms a stable alpha-helical homotrimer. In some embodiments, the C-terminal helix-forming segment comprises between about 12 and about 26 residues.

In some embodiments, the segment comprises (1) an amino acid substitution at position M463 relative to SEQ ID NO: 499, wherein M is substituted with any one of L, I, V, (2) an amino acid substitution at position N464 relative to SEQ ID NO: 499, wherein N is substituted with N, (3) an amino acid substitution at position Q465 relative to SEQ ID NO: 499, wherein Q is substituted with any one of Q, T, N, D, E, S, K, R, A, (4) an amino acid substitution at position S466 relative to SEQ ID NO: 499, wherein S is substituted with S, (5) an amino acid substitution at position L467 relative to SEQ ID NO: 499, wherein L is substituted with any one of I, V, L, A, (6) an amino acid substitution at position Q468 relative to SEQ ID NO: 499, wherein Q is substituted with any one of S, K, T, D, E, R, Q, (7) an amino acid substitution at position Q469 relative to SEQ ID NO: 499, wherein Q is substituted with any one of S, Q, N, E, A, D, K, T, R, (8) an amino acid substitution at position S470 relative to SEQ ID NO: 499, wherein S is substituted with any one of L, A, I, V, N, (9) an amino acid substitution at position K471 relative to SEQ ID NO: 499, wherein K is substituted with any one of E, Q, S, R, K, A, T, D, L, N, (10) an amino acid substitution at position D472 relative to SEQ ID NO: 499, wherein D is substituted with any one of K, T, E, Q, D, N, S, A, (11) an amino acid substitution at position Y473 relative to SEQ ID NO: 499, wherein Y is substituted with any one of A, S, R, T, V, E, I, K, L, D, Q, (12) an amino acid substitution at position I474 relative to SEQ ID NO: 499, wherein I is substituted with any one of L, I, V, (13) an amino acid substitution at position K475 relative to SEQ ID NO: 499, wherein K is substituted with any one of K, H, D, T, A, S, R, Q, E, N, (14) an amino acid substitution at position E476 relative to SEQ ID NO: 499, wherein E is substituted with any one of K, R, S, E, A, T, H, D, (15) an amino acid substitution at position A477 relative to SEQ ID NO: 499, wherein A is substituted with any one of A, L, I, V, (16) an amino acid substitution at position Q478 relative to SEQ ID NO: 499, wherein Q is substituted with any one of E, L, T, R, K, Q, S, I, (17) an amino acid substitution at position R479 relative to SEQ ID NO: 499, wherein R is substituted with any one of K, N, E, Q, S, T, H, R, A, (18) an amino acid substitution at position L480 relative to SEQ ID NO: 499, wherein L is substituted with any one of D, L, E, K, T, R, V, I, Q, (19) an amino acid substitution at position L481 relative to SEQ ID NO: 499, wherein L is substituted with any one of L, V, I, (20) an amino acid substitution at position D482 relative to SEQ ID NO: 499, wherein D is substituted with any one of E, K, N, D, L, Q, H, (21) an amino acid substitution at position T483 relative to SEQ ID NO: 499, wherein T is substituted with any one of E, K, S, Q, A, T, (22) an amino acid substitution at position V484 relative to SEQ ID NO: 499, wherein V is substituted with any one of V, L, I, (23) an amino acid substitution at position N485 relative to SEQ ID NO: 499, wherein N is substituted with any one of R, K, L, V, E, Q, I, (24) an amino acid substitution at position P486 relative to SEQ ID NO: 499, wherein P is substituted with any one of R, E, A, S, L, (25) an amino acid substitution at position S487 relative to SEQ ID NO: 499, wherein S is substituted with any one of Q, R, T, S, L, (26) an amino acid substitution at position L488 relative to SEQ ID NO: 499, wherein L is substituted with any one of L, and/or (27) any combination of (1)-(26).

In some embodiments, the segment comprises a polypeptide sequence listed in Table 10D, or a polypeptide sequence having between 1 and 5 amino acid substitutions thereto.

In some embodiments, the recombinant polypeptide comprises an engineered ectodomain of a Respiratory Syncytial Virus (RSV) fusion (F) protein, wherein the ectodomain comprises (a) a C-terminal helix-forming segment, between about residue 500 and about residue 530 relative to SEQ ID NO: 1, comprising one or more amino acid substitutions selected such that the segment forms a stable alpha-helical homotrimer, (b) one, two, three or more amino acid substitutions at positions 140, 399, 400, 485, 486, 487, 488, 489, 494, or 498 relative to SEQ ID NO: 1, (c) one, two, three or more amino acid substitutions at positions 56, 58, 154, 187, 296, or 298 relative to SEQ ID NO: 1, (d) one, two, three or more amino acid substitutions at positions 75, 216, 218, or 219 relative to SEQ ID NO: 1, (e) one, two, three or more amino acid substitutions at positions 92, 232, 235, 238, 249, 250, or 254 relative to SEQ ID NO: 1, (f) one, two, three or more amino acid substitutions at positions 67, 137, or 339 relative to SEQ ID NO: 1, (g) a substitution of a non-cleavable linker in place of a furin cleavage site at about residue 100 to about residue 140 relative to SEQ ID NO: 1 or (h) any combination of (a)-(g).

In some embodiments, the ectodomain comprises the C-terminal helix-forming segment, between about residue 500 and about residue 530 relative to SEQ ID NO: 1, comprising one or more amino acid substitutions selected such that the segment forms a stable alpha-helical homotrimer. In some embodiments, the C-terminal helix-forming segment comprises between about 10 and about 30 residues.

In some embodiments, the segment comprises substitutions relative to the reference sequence SEQ ID NO: 1 at two or more, three or more, or four or more residues that generate hydrophobic contacts between the segments in the alpha-helical homotrimer.

In some embodiments, the segment comprises (1) an amino acid substitution at position F505 relative to SEQ ID NO: 1, wherein F is substituted with A, I, L, M, V, G, T; (2) an amino acid substitution at position I506 relative to SEQ ID NO: 1, wherein I is substituted with any amino acids except P, preferably D, E, K, N, Q, R, S, T, Y or A, I, L, V; (3) an amino acid substitution at position R507 relative to SEQ ID NO: 1, wherein R is substituted with any amino acids except P, preferably D, E, K, N, Q, R, S, T, Y or A, I, L, V; (4) an amino acid substitution at position K508 relative to SEQ ID NO: 1, wherein R is substituted with K, Q, R, preferably A, V, T, I; (5) an amino acid substitution at position S509 relative to SEQ ID NO: 1, wherein S is substituted with A, I, L, M, V, F, W, Y, G, T, preferably A, I, L, M, V; (6) an amino acid substitution at position D510 relative to SEQ ID NO: 1, wherein D is substituted with any amino acids, preferably D, E, K, N, Q, R, S, T, Y; (7) an amino acid substitution at position E511 relative to SEQ ID NO: 1, wherein E is substituted with any amino acids; (8) an amino acid substitution at position L512 relative to SEQ ID NO: 1, wherein L is substituted with D, E, K, N, Q, R, S, T, Y, preferably A, I, L, M, V, F, W, Y, G, T; (9) an amino acid substitution at position L513 relative to SEQ ID NO: 1, wherein L is substituted with any amino acids, preferably A, I, L, M, V, F, W, Y, G, more preferably D, E, K, N, Q, R, S, T, Y; (10) an amino acid substitution at position H514 relative to SEQ ID NO: 1, wherein H is substituted with any amino acids except P, preferably D, E, K, N, Q, R, S, T, Y; (11) an amino acid substitution at position N515 relative to SEQ ID NO: 1, wherein N is substituted with any amino acids except P, preferably A, I, L, M, V, F, W, Y, G; (12) an amino acid substitution at position V516 relative to SEQ ID NO: 1, wherein V is substituted with A, I, L, M, V, F, W, Y, G, or T, S, K; (13) an amino acid substitution at position N517 relative to SEQ ID NO: 1, wherein N is substituted with any amino acid except P, preferably D, E, K, N, Q, R, S, T, Y; (14) an amino acid substitution at position T518 relative to SEQ ID NO: 1, wherein T is substituted with Any except P, preferably D, E, K, N, Q, R, S, T, Y; (15) an amino acid substitution at position G519 relative to SEQ ID NO: 1, wherein G is substituted with any amino acid except P, preferably D, E, K, N, Q, R, S, T, Y; and/or (16) any combination of (1)-(15).

In some embodiments, the segment comprises (1) an amino acid substitution at position L503 relative to SEQ ID NO: 1, wherein F is substituted with Q, V, K, R, N, L, (2) an amino acid substitution at position A504 relative to SEQ ID NO: 1, wherein I is substituted with any amino acids except P, preferably S, T, L, A, Q, K, E, Y, (3) an amino acid substitution at position F505 relative to SEQ ID NO: 1, wherein F is substituted with I, V, N, T, L, (4) an amino acid substitution at position I506 relative to SEQ ID NO: 1, wherein I is substituted with any amino acids except P, preferably Q, N, K, R, V, S, (5) an amino acid substitution at position R507 relative to SEQ ID NO: 1, wherein R is substituted with any amino acids except P, preferably A, N, K, E, D, Q, (6) an amino acid substitution at position K508 relative to SEQ ID NO: 1, wherein R is substituted with T, M, V, R, (7) an amino acid substitution at position S509 relative to SEQ ID NO: 1, wherein S is substituted with T, I, K, Q, M, E, V, S, (8) an amino acid substitution at position D510 relative to SEQ ID NO: 1, wherein D is substituted with S, K, N, D, E, (9) an amino acid substitution at position E511 relative to SEQ ID NO: 1, wherein E is substituted with R, S, E, K, A, T, L, (10) an amino acid substitution at position L512 relative to SEQ ID NO: 1, wherein L is substituted with V, N, T, L, (11) an amino acid substitution at position L513 relative to SEQ ID NO: 1, wherein L is substituted with D, T, H, K, E, N, R, (12) an amino acid substitution at position H514 relative to SEQ ID NO: 1, wherein H is substituted with A, N, E, S, V, K, T, D, (13) an amino acid substitution at position N515 relative to SEQ ID NO: 1, wherein N is substituted with I, E, L, T, Q, (14) an amino acid substitution at position V516 relative to SEQ ID NO: 1, wherein V is substituted with E, I, K, N, R, Q, (15) an amino acid substitution at position N517 relative to SEQ ID NO: 1, wherein N is substituted with A, S, K, E, R, (16) an amino acid substitution at position T518 relative to SEQ ID NO: 1, wherein Tis substituted with K, S, Q, R, D, E, (17) an amino acid substitution at position G519 relative to SEQ ID NO: 1, wherein G is substituted with V, L, I, (18) an amino acid substitution at position I520 relative to SEQ ID NO: 1, wherein G is substituted with K, Q, E, N, T, (19) an amino acid substitution at position P521 relative to SEQ ID NO: 1, wherein G is substituted with H, D, E, K, R, N, Q, (20) an amino acid substitution at position E522 relative to SEQ ID NO: 1, wherein G is substituted with L, R, I, V, (21) an amino acid substitution at position A523 relative to SEQ ID NO: 1, wherein G is substituted with E, V, L, K, R I, (22) an amino acid substitution at position P524 relative to SEQ ID NO: 1, wherein G is substituted with A, K, T, E, R, (23) an amino acid substitution at position R525 relative to SEQ ID NO: 1, wherein G is substituted with H, R, S, L, N, E, D, (24) an amino acid substitution at position D526 relative to SEQ ID NO: 1, wherein G is substituted with I, L, V, R, (25) an amino acid substitution at position G527 relative to SEQ ID NO: 1, wherein G is substituted with E, K, Q, D, (26) an amino acid substitution at position Q528 relative to SEQ ID NO: 1, wherein G is substituted with D, K, S, R, A, (27) an amino acid substitution at position A529 relative to SEQ ID NO: 1, wherein G is substituted with T, L, (28) an amino acid substitution at position Y530 relative to SEQ ID NO: 1, wherein G is substituted with L, E, T, (29) an amino acid substitution at position V531 relative to SEQ ID NO: 1, wherein G is substituted with A, R, K, (30) an amino acid substitution at position R532 relative to SEQ ID NO: 1, wherein G is substituted with V, A, and/or (31) any combination of (1)-(30).

In some embodiments, the segment comprises a polypeptide sequence listed in Table 2B or Table 2C, or a polypeptide sequence having between 1 and 5 amino acid substitutions thereto. In some embodiments, the segment comprises the polypeptide sequence NQSREIIRAINIVRKIASEK (SEQ ID NO: 10), NQSALWLEAAKYVKQAREKS (SEQ ID NO: 11), NQSAKNAEAAKIAEETKRKD (SEQ ID NO: 12), or NQSRETAKAVSAVK (SEQ ID NO: 75), or a polypeptide sequence having between 1 and 5 amino acid substitutions thereto. In some embodiments, the segment comprises the polypeptide sequence NQSREIIRAINIVRKIASEK (SEQ ID NO: 10) or a polypeptide sequence having between 1 and 5 amino acid substitutions thereto. In some embodiments, the segment comprises the polypeptide sequence NQSREIIRAINIVRKIASEK (SEQ ID NO: 10).

In some embodiments, the ectodomain comprises (b) one, two, three or more amino acid substitutions at positions 140, 399, 400, 485, 486, 487, 488, 489, 494, or 498 relative to SEQ ID NO: 1. In some embodiments, the ectodomain comprises one or more of the following sets of amino acid substitutions relative to SEQ ID NO: 1: E487R+K498A, E487R+K498E, E487K+K498E, D486A+E487R+K498A, D486Q+E487R+K498A, D486E+E487A+D489A+T400D, D486A+E487M+K498A, E487Q, D486S, F488W+D489A+T400D+E487R+K498A, F140W+D489A+T400D+E487R+K498A, Q494I+S485I+K399A+487R+498A, Q494M+S485I+K399A; D486A+487M+498A, Q494L+S485A+K399V+D486A+487M+498A, Q494M+S485A+K399V+D486A+487M+498A, Q494A+S485F+K399V+D486A+487M+498Y, D489A+T400D+E487R+K498A, or D489A+T400D. In some embodiments, the ectodomain comprises the amino acid substitutions D489A, T400D, E487R, and K498A. In some embodiments, the ectodomain comprises the amino acid substitutions F488W, D489A, T400D, E487R, K498A, and D486A. In some embodiments, the ectodomain comprises the amino acid substitutions F488W, D489A, T400D, E487R, K498A, and T249P.

In some embodiments, the polypeptide comprises, C-terminal to the ectodomain, a heterologous multimerization domain. In some embodiments, the multimerization domain is a trimerization domain. In some embodiments, the multimerization domain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64). In some embodiments, the ectodomain comprises the amino acid substitutions S155C, S290C, S190F, and V207L.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 6, below, optionally lacking a p27 peptide shown in bold, and in which “X” refers to sites involving an added C-terminal helical segment and can be any amino acid:

(SEQ ID NO: 6)
QNITEEFYQSTCSAVSRGYLSALRTGWYTSVITIELSNIKETKCNGTDTKVKLIKQEL
DKYKNAVTELQLLMQNTPAVNNRARREAPQYMNYTINTTKNLNVSISKKRKRRFLGFL
LGVGSAIASGIAVCKVLHLEGEVNKIKNALQLTNKAVVSLSNGVSVLTFRVLDLKNYI
NNQLLPMLNRQSCRISNIETVIEFQQKNSRLLEITREFSVNAGVTTPLSTYMLTNSEL
LSLINDMPITNDQKKLMSSNVQIVRQQSYSIMCIIKEEVLAYVVQLPIYGVIDTPCWK
LHTSPLCTTNIKEGSNICLTRTDRGWYCDNAGSVSFFPQADTCKVQSNRVFCDTMNSL
TLPSEVSLCNTDIFNSKYDCKIMTSKTDISSSVITSLGAIVSCYGKTKCTASNKNRGI
IKTFSNGCDYVSNKGVDTVSVGNTLYYVNKLEGKNLYVKGEPIINYYDPLVFPSDEFD
ASISQVNEKINQSXXXXXXXXXXXXXXXXX.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 7, below, optionally lacking a p27 peptide shown in bold, in which “X” refers to sites involving an added C-terminal helical segment and can be any amino acid:

(SEQ ID NO: 7)
QNITEEFYQSTCSAVSKGYLSALRTGWYTSVITIELSNIKENKCNGTDAKVKLIKQEL
DKYKNAVTELQLLMQSTPATNNRARRELPRFMNYTLNNAKKTNVTLSKKRKRRFLGFL
LGVGSAIASGVAVCKVLHLEGEVNKIKSALLSTNKAVVSLSNGVSVLTFKVLDLKNYI
DKQLLPILNKQSCSISNIETVIEFQQKNNRLLEITREFSVNAGVTTPVSTYMLINSEL
LSLINDMPITNDQKKLMSNNVQIVRQQSYSIMCIIKEEVLAYVVQLPLYGVIDTPCWK
LHTSPLCTTNTKEGSNICLTRTDRGWYCDNAGSVSFFPQAETCKVQSNRVFCDTMNSL
TLPSEVNLCNVDIFNPKYDCKIMTSKTDVSSSVITSLGAIVSCYGKTKCTASNKNRGI
IKTFSNGCDYVSNKGVDTVSVGNTLYYVNKQEGKSLYVKGEPIINFYDPLVFPSDEFD
ASISQVNEKINQSXXXXXXXXXXXXXXXXX.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 8, below, optionally lacking a p27 peptide shown in bold:

(SEQ ID NO: 8)
QNITEEFYQSTCSAVSRGYLSALRTGWYTSVITIELSNIKETKCNGTDTKVKLIKQEL
DKYKNAVTELQLLMQNTPAVNNRARREAPQYMNYTINTTKNLNVSISKKRKRRFLGFL
LGVGSAIASGIAVCKVLHLEGEVNKIKNALQLTNKAVVSLSNGVSVLTFRVLDLKNYI
NNQLLPMLNRQSCRISNIETVIEFQQKNSRLLEITREFSVNAGVTTPLSTYMLTNSEL
LSLINDMPITNDQKKLMSSNVQIVRQQSYSIMCIIKEEVLAYVVQLPIYGVIDTPCWK
LHTSPLCTTNIKEGSNICLTRTDRGWYCDNAGSVSFFPQADTCKVQSNRVFCDTMNSL
TLPSEVSLCNTDIFNSKYDCKIMTSKTDISSSVITSLGAIVSCYGKTKCTASNKNRGI
IKTFSNGCDYVSNKGVDTVSVGNTLYYVNKLEGKNLYVKGEPIINYYDPLVFPSDEFD
ASISQVNEKINQSREIIRAINIVRKIASEK.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 9, below, optionally lacking a p27 peptide shown in bold:

(SEQ ID NO: 9)
QNITEEFYQSTCSAVSKGYLSALRTGWYTSVITIELSNIKENKCNGTDAKVKLIKQEL
DKYKNAVTELQLLMQSTPATNNRARRELPRFMNYTLNNAKKTNVTLSKKRKRRFLGFL
LGVGSAIASGVAVCKVLHLEGEVNKIKSALLSTNKAVVSLSNGVSVLTFKVLDLKNYI
DKQLLPILNKQSCSISNIETVIEFQQKNNRLLEITREFSVNAGVTTPVSTYMLINSEL
LSLINDMPITNDQKKLMSNNVQIVRQQSYSIMCIIKEEVLAYVVQLPLYGVIDTPCWK
LHTSPLCTTNTKEGSNICLTRTDRGWYCDNAGSVSFFPQAETCKVQSNRVFCDTMNSL
TLPSEVNLCNVDIFNPKYDCKIMTSKTDVSSSVITSLGAIVSCYGKTKCTASNKNRGI
IKTFSNGCDYVSNKGVDTVSVGNTLYYVNKQEGKSLYVKGEPIINFYDPLVFPSDEFD
ASISQVNEKINQSREIIRAINIVRKIASEK.

In some embodiments, the polypeptide comprises a sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 1-9.

In another aspect, the disclosure provides a trimeric protein complex comprising a polypeptide disclosed herein. In some embodiments, the thermal stability, assayed by nanoDSF, is increased by at least 10° C., at least 15° C., at least 20° C., about 10° C. to about 30° C., about 10° C. to about 20° C., or about 20° C. to about 30° C. compared to a trimeric protein complex lacking modifications (a)-(h). In some embodiments, the stability, assayed by storage at about 40° C., is increased compared to a trimeric protein complex lacking modifications (a)-(h). In some embodiments, the thermal stability is increased compared to a reference RSV F protein comprising amino acid substitutions consisting essentially of S155C, S290C, S190F, and V207L (DS-Cav1). In some embodiments, a thermal stability, assayed by nanoDSF, is increased by at least 10° C., at least 15° C., at least 20° C., about 10° C. to about 30° C., about 10° C. to about 20° C., or about 20° C. to about 30° C. compared to a trimeric protein complex lacking modifications (a)-(g).

In some embodiments, the stability, assayed by storage at about 40° C., is increased compared to a trimeric protein complex lacking modifications (a)-(g).

In another aspect, the disclosure provides a protein nanostructure comprising a trimeric component comprising a polypeptide disclosed herein. In some embodiments, the nanostructure is a two-component nanostructure comprising the first, trimeric component and a second, pentameric component. In some embodiments, the first, trimeric component comprises an engineered ectodomain of a Respiratory Syncytial Virus (RSV) fusion (F) polypeptide and an I53-50A polypeptide. In some embodiments, the first, trimeric component comprises a fusion protein comprising, in N- to C-terminal order, the RSV fusion (F) polypeptide, an amino acid linker, and the I53-50A polypeptide. In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, and V207L relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, D489A, T400D, E487R, and K498A relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, F488W, D489A, T400D, E487R, K498A, and T249P relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, F488W, D489A, T400D, E487R, K498A, and D486A relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the trimeric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of the sequences listed in Table 19. In some embodiments, the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 20, 44, 45, 52, 71, 73, 74.

In another aspect, the disclosure provides a pharmaceutical composition comprising a polypeptide, a protein complex, or a nanostructure disclosed herein. In another aspect, the disclosure provides a vaccine comprising a polypeptide, a protein complex, or a nanostructure disclosed herein. In another aspect, the disclosure provides a trimeric protein complex comprising a polypeptide disclosed herein. In another aspect, the disclosure provides a protein nanostructure comprising a trimeric component comprising a polypeptide disclosed herein. In some embodiments, the nanostructure is a two-component nanostructure comprising the first, trimeric component and a second, pentameric component. In some embodiments, the first, trimeric component comprises an engineered ectodomain of a Respiratory Syncytial Virus (RSV) fusion (F) polypeptide and an I53-50A polypeptide. In some embodiments the first, trimeric component comprises an engineered ectodomain of a human Metapneumovirus (hMPV) fusion (F) polypeptide and an I53-50A polypeptide. In some embodiments the first, trimeric component comprises an engineered ectodomain of a hMPV/A fusion (F) polypeptide and an I53-50A polypeptide. In some embodiments the first, trimeric component comprises an engineered ectodomain of a hMPV/B fusion (F) polypeptide and an I53-50A polypeptide. In some embodiments the first, trimeric component comprises an engineered ectodomain of a human Parainfluenza virus type 3 (PIV3) fusion (F) polypeptide and an I53-50A polypeptide. In some embodiments the first, trimeric component comprises an engineered ectodomain of a human Parainfluenza virus type 5 (PIV3) fusion (F) polypeptide and an I53-50A polypeptide. In some embodiments the first, trimeric component comprises an engineered ectodomain of a SARS-COV-2 spike(S) polypeptide and an I53-50A polypeptide. In some embodiments the first, trimeric component comprises an engineered ectodomain of a Nipah virus fusion (F) polypeptide and an I53-50A polypeptide. In some embodiments the first, trimeric component comprises a fusion protein comprising, in N- to C-terminal order, the engineered fusion (F) polypeptide, an amino acid linker, and the I53-50A polypeptide. In some embodiments the first, trimeric component comprises a fusion protein comprising, in N- to C-terminal order, the engineered spike(S) polypeptide, an amino acid linker, and the I53-50A polypeptide. In some embodiments, the trimeric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of the sequences listed in Table 19 or to any one of the sequences listed in Table 19 without the underlined and/or bold/italicized polypeptide sequences. In some embodiments the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 20, 44, 45, 52, 71, 73, 74.

In another aspect, the disclosure provides a method of vaccinating a subject, comprising administering to the subject a composition disclosed herein. In another aspect, the disclosure provides a method of generating an immune response in a subject, comprising administering to the subject a composition disclosed herein. In another aspect, the disclosure provides a method of treating or preventing a viral infection in a subject, comprising administering to the subject a composition disclosed herein. In another aspect, the disclosure provides a composition for use in vaccinating, generating an immune response, or treating or preventing any viral infection disease disclosed herein. In another aspect, the disclosure provides a composition, method, or use as described herein. In another aspect, the disclosure provides a method of making a composition, comprising culturing host cells modified to express one or more polypeptides as described herein.

In another aspect, the disclosure provides a recombinant polypeptide for use in displaying a molecule such as an antigen, comprising an alpha-helical segment and a multimerization domain, wherein the alpha-helical segment comprises one or more amino acid substitutions selected such that the segment forms a stable alpha-helical homotrimer. In some embodiments, the alpha-helical segment has improved hydrophobic packing. In some embodiments, the alpha-helical segment comprises between about 7 and about 31 residues. In some embodiments, the amino acid substitutions comprise polar, charged and/or hydrophobic amino acids.

In some embodiments, the alpha-helical segment comprises a polypeptide sequence according to any one of LXXTIXXLLXIXXXLXXXL (SEQ ID NO: 566), LVXTXKXLXDLIXXLXXLLXKLXX (SEQ ID NO: 567), LNKVKKXVXXLXXXVXXLEKXLX (SEQ ID NO: 568), EKIXXAIKKAXKL (SEQ ID NO: 569), EXIXKAIKXLXXXXX (SEQ ID NO: 570), XKXXEXXXXVXXXXXXXXX (SEQ ID NO: 571), XXLKKAAXIXKKXLKXX (SEQ ID NO: 572).

In some embodiments, the alpha-helical segment comprises a polypeptide sequence according to any one of the consensus sequences in Table 24.

In some embodiments, the alpha-helical segment comprises a polypeptide sequence according to a) L X₂ X₂ T I X₂ X₂ L L X₂ I [V/I] X₂ X₂ L [I/L] X₂ X₂ L (SEQ ID NO: 573), b) L V [A/T] T X₂ K X₂ L X₂ D L I X₂ X₂ L [K/E] X₂ L L X₂ KL X₂ X₂ (SEQ ID NO: 574), or c) LN K V K K X₂ V X₂ X₂ L X₂ X₂ X₂ V X₂ X₂ L E K X₂ L X₂ (SEQ ID NO: 575), wherein X₂ is polar and charged residues selected from S, T, N, Q, E, D, R, K, and H, preferably wild type amino acid.

In some embodiments, the alpha-helical segment comprises a polypeptide sequence according a) E K I X₂ X₂ A I K K A X₂ KL (SEQ ID NO: 576), b) E X₂ I X₂ K A I K X₂ L [L/X₂] X₂ X₂ [X₁/X₂] X₂ (SEQ ID NO: 577), and c) X₂ K [X₁/T] [L/E] E [T/A] X₁ X₂ [I/X₂] V X₂ X₂ [X₁/X₂] [X₁/X₂] X₂ X₂ X₁ X₂ X₂ (SEQ ID NO: 578), or d) X₂ X₂ L K K A A X₂ I X₁ K K X₁ L K X₂ X₂ (SEQ ID NO: 579) wherein X₁ is apolar residues selected from A, I, L, and M, and wherein X₂ is polar and charged residues selected from S, T, N, Q, E, D, R, K, and H, preferably wild type amino acid.

In some embodiments, the alpha-helical segment comprises a polypeptide sequence listed in Table 25A or Table 25B or a polypeptide sequence having between 1 and 5 amino acid substitutions thereto. In some embodiments, the alpha-helical segment comprises the polypeptide sequence NQSREIIRAINIVRKIASEK (SEQ ID NO: 10), NQSALWLEAAKYVKQAREKS (SEQ ID NO: 11), NQSAKNAEAAKIAEETKRKD (SEQ ID NO: 12), or NQSRETAKAVSAVK (SEQ ID NO: 75), or the polypeptide sequence having between 1 and 5 amino acid substitutions thereto.

In some embodiments, the multimerization domain is I53-50A or a variant thereof. In some embodiments, the multimerization domain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64). In some embodiments, the polypeptide comprises an N-terminal fusion of the alpha-helical segment to the multimerization domain via a peptide bond or polypeptide linker. In some embodiments, the polypeptide comprises, N-terminal to the alpha-helical segment, an antigen polypeptide.

In another aspect, the disclosure provides a polypeptide comprising an alpha-helical segment, comprising a polypeptide sequence listed in Table 25A or Table 25B, or a polypeptide sequence having between 1 and 5 amino acid substitutions thereto. In another aspect, the disclosure provides a protein nanostructure comprising a trimeric component comprising a polypeptide described herein. In some embodiments, the nanostructure is a two-component nanostructure comprising the first, trimeric component and a second, pentameric component. In some embodiments, the nanostructure is a two-component nanostructure comprising a second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In another aspect, the disclosure provides a pharmaceutical composition comprising a polypeptide or a nanostructure described herein. In another aspect, the disclosure provides a vaccine comprising a polypeptide or a nanostructure described herein. In another aspect, the disclosure provides a method of vaccinating a subject, comprising administering to the subject a composition described herein. In another aspect, the disclosure provides a method of generating an immune response or treating or preventing a viral infection in a subject, comprising administering to the subject a polypeptide or a nanostructure described herein.

In another aspect, the disclosure provides a method of making a polypeptide or a nanostructure described herein, comprising culturing host cells modified to express one or more polypeptides as described herein.

In another aspect, the disclosure provides a recombinant polypeptide, comprising an engineered ectodomain of a Respiratory Syncytial Virus (RSV) fusion (F) protein, wherein the ectodomain comprises: (1) one, two, three or more amino acid substitutions at positions 140, 399, 400, 485, 486, 487, 488, 489, 494, or 498 relative to SEQ ID NO: 1, (2) one, two, three or more amino acid substitutions at positions 56, 58, 154, 187, 296, or 298 relative to SEQ ID NO: 1, (3) one, two, three or more amino acid substitutions at positions 75, 216, 218, or 219 relative to SEQ ID NO: 1, (4) one, two, three or more amino acid substitutions at positions 92, 232, 235, 238, 249, 250, or 254 relative to SEQ ID NO: 1, (5) one, two, three or more amino acid substitutions at positions 67, 137, or 339 relative to SEQ ID NO: 1, (6) a substitution of a non-cleavable linker in place of a furin cleavage site at about residue 100 to about residue 140 relative to SEQ ID NO: 1, or (7) any combination of (1)-(6).

In some embodiments, the ectodomain comprises one, two, three or more amino acid substitutions at positions 140, 399, 400, 485, 486, 487, 488, 489, 494, or 498 relative to SEQ ID NO: 1.

In some embodiments, the ectodomain comprises one or more of the following sets of amino acid substitutions relative to SEQ ID NO: 1:: E487R+K498A, E487R+K498E, E487K+K498E, D486A+E487R+K498A, D486Q+E487R+K498A, D486E+E487A+D489A+T400D, D486A+E487M+K498A, E487Q, D486S, F488W+D489A+T400D+E487R+K498A, F140W+D489A+T400D+E487R+K498A, Q494I+S485I+K399A+487R+498A, Q494M+S485I+K399A; D486A+487M+498A, Q494L+S485A+K399V+D486A+487M+498A, Q494M+S485A+K399V+D486A+487M+498A, Q494A+S485F+K399V+D486A+487M+498Y, D489A+T400D+E487R+K498A, or D489A+T400D.

In some embodiments, the ectodomain comprises the amino acid substitutions D489A, T400D, E487R, and K498A. In some embodiments, the ectodomain comprises the amino acid substitutions F488W, D489A, T400D, E487R, K498A, and D486A. In some embodiments, the ectodomain comprises the amino acid substitutions F488W, D489A, T400D, E487R, K498A, and T249P.

In some embodiments, the polypeptide comprises a heterologous multimerization domain. In some embodiments, the multimerization domain is a trimerization domain. In some embodiments, the multimerization domain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64). In some embodiments, the ectodomain comprises the amino acid substitutions S155C, S290C, S190F, and V207L.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 6, below, optionally lacking a p27 peptide shown in bold, and in which “X” refers to sites involving an added C-terminal helical segment and can be any amino acid:

(SEQ ID NO: 6)
QNITEEFYQSTCSAVSRGYLSALRTGWYTSVITIELSNIKETKCNGTDTKVKLIKQEL
DKYKNAVTELQLLMQNTPAVNNRARREAPQYMNYTINTTKNLNVSISKKRKRRFLGFL
LGVGSAIASGIAVCKVLHLEGEVNKIKNALQLTNKAVVSLSNGVSVLTFRVLDLKNYI
NNQLLPMLNRQSCRISNIETVIEFQQKNSRLLEITREFSVNAGVTTPLSTYMLTNSEL
LSLINDMPITNDQKKLMSSNVQIVRQQSYSIMCIIKEEVLAYVVQLPIYGVIDTPCWK
LHTSPLCTTNIKEGSNICLTRTDRGWYCDNAGSVSFFPQADTCKVQSNRVFCDTMNSL
TLPSEVSLCNTDIFNSKYDCKIMTSKTDISSSVITSLGAIVSCYGKTKCTASNKNRGI
IKTFSNGCDYVSNKGVDTVSVGNTLYYVNKLEGKNLYVKGEPIINYYDPLVFPSDEED
ASISQVNEKINQSXXXXXXXXXXXXXXXXX.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 7, below, optionally lacking a p27 peptide shown in bold, in which “X” refers to sites involving an added C-terminal helical segment and can be any amino acid:

(SEQ ID NO: 7)
QNITEEFYQSTCSAVSKGYLSALRTGWYTSVITIELSNIKENKCNGTDAKVKLIKQEL
DKYKNAVTELQLLMQSTPATNNRARRELPRFMNYTLNNAKKTNVTLSKKRKRRFLGFL
LGVGSAIASGVAVCKVLHLEGEVNKIKSALLSTNKAVVSLSNGVSVLTFKVLDLKNYI
DKQLLPILNKQSCSISNIETVIEFQQKNNRLLEITREFSVNAGVTTPVSTYMLTNSEL
LSLINDMPITNDQKKLMSNNVQIVRQQSYSIMCIIKEEVLAYVVQLPLYGVIDTPCWK
LHTSPLCTTNTKEGSNICLTRTDRGWYCDNAGSVSFFPQAETCKVQSNRVFCDTMNSL
TLPSEVNLCNVDIFNPKYDCKIMTSKTDVSSSVITSLGAIVSCYGKTKCTASNKNRGI
IKTFSNGCDYVSNKGVDTVSVGNTLYYVNKQEGKSLYVKGEPIINFYDPLVFPSDEFD
ASISQVNEKINQSXXXXXXXXXXXXXXXXX.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 8, below, optionally lacking a p27 peptide shown in bold:

(SEQ ID NO: 8)
QNITEEFYQSTCSAVSRGYLSALRTGWYTSVITIELSNIKETKCNGTDTKVKLIKQELDKYKNA
VTELQLLMQNTPAVNNRARREAPQYMNYTINTTKNLNVSISKKRKRRFLGFLLGVGSAIASGIA
VCKVLHLEGEVNKIKNALQLTNKAVVSLSNGVSVLTFRVLDLKNYINNQLLPMLNRQSCRISNI
ETVIEFQQKNSRLLEITREFSVNAGVTTPLSTYMLTNSELLSLINDMPITNDQKKLMSSNVQIV
RQQSYSIMCIIKEEVLAYVVQLPIYGVIDTPCWKLHTSPLCTTNIKEGSNICLTRTDRGWYCDN
AGSVSFFPQADTCKVQSNRVFCDTMNSLTLPSEVSLCNTDIFNSKYDCKIMTSKTDISSSVITS
LGAIVSCYGKTKCTASNKNRGIIKTFSNGCDYVSNKGVDTVSVGNTLYYVNKLEGKNLYVKGEP
IINYYDPLVFPSDEFDASISQVNEKINQSREIIRAINIVRKIASEK.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 9, below, optionally lacking a p27 peptide shown in bold:

(SEQ ID NO: 9)
QNITEEFYQSTCSAVSKGYLSALRTGWYTSVITIELSNIKENKCNGTDAKVKLIKQELDKYKNA
VTELQLLMQSTPATNNRARRELPRFMNYTLNNAKKTNVTLSKKRKRRFLGFLLGVGSAIASGVA
VCKVLHLEGEVNKIKSALLSTNKAVVSLSNGVSVLTFKVLDLKNYIDKQLLPILNKQSCSISNI
ETVIEFQQKNNRLLEITREFSVNAGVTTPVSTYMLINSELLSLINDMPITNDQKKLMSNNVQIV
RQQSYSIMCIIKEEVLAYVVQLPLYGVIDTPCWKLHTSPLCTINTKEGSNICLTRTDRGWYCDN
AGSVSFFPQAETCKVQSNRVFCDTMNSLTLPSEVNLCNVDIFNPKYDCKIMTSKTDVSSSVITS
LGAIVSCYGKTKCTASNKNRGIIKTFSNGCDYVSNKGVDTVSVGNTLYYVNKQEGKSLYVKGEP
IINFYDPLVFPSDEFDASISQVNEKINQSREIIRAINIVRKIASEK.

In some embodiments, the polypeptide comprises a sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 1-9.

In another aspect, the disclosure provides a trimeric protein complex comprising a polypeptide described herein.

In some embodiments, a thermal stability, assayed by nanoDSF, is increased by at least 10° C., at least 15° C., at least 20° C., about 10° C. to about 30° C., about 10° C. to about 20° C., or about 20° C. to about 30° C. compared to a trimeric protein complex lacking modifications (1)-(7).

In some embodiments, the stability, assayed by storage at about 40° C., is increased compared to a trimeric protein complex lacking modifications (1)-(7). In some embodiments, the thermal stability is increased compared to a reference RSV F protein comprising amino acid substitutions consisting essentially of S155C, S290C, S190F, and V207L (DS-Cav1).

In another aspect, the disclosure provides a protein nanostructure comprising a trimeric component comprising a polypeptide described herein. In some embodiments, the nanostructure is a two-component nanostructure comprising the first, trimeric component and a second, pentameric component. In some embodiments, the first, trimeric component comprises an engineered ectodomain of a Respiratory Syncytial Virus (RSV) fusion (F) polypeptide and an I53-50A polypeptide. In some embodiments, the first, trimeric component comprises a fusion protein comprising, in N- to C-terminal order, the RSV fusion (F) polypeptide, an amino acid linker, and the I53-50A polypeptide.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, and V207L relative to SEQ ID NO: 1, and an multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an e engineered ectodomain of a RSV F polypeptide comprising an amino acid substitutions at position S155C, S290C, S190F, V207L, D489A, T400D, E487R, and K498A relative to SEQ ID NO: 1, and an multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, F488W, D489A, T400D, E487R, K498A, and T249P relative to SEQ ID NO: 1, and an multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64). and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, F488W, D489A, T400D, E487R, K498A, and D486A relative to SEQ ID NO: 1, and an multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the trimeric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of sequence listed in Table 19 or to any one of the sequences listed in Table 19 without the underlined and/or bold/italicized polypeptide sequences.

In some embodiments, the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 20, 44, 45, 52, 71, 73, 74.

In another aspect, the disclosure provides a pharmaceutical composition comprising a polypeptide, a protein complex, or a nanostructure described herein. In another aspect, the disclosure provides a vaccine composition comprising a polypeptide, a protein complex, or a nanostructure described herein. In another aspect, the disclosure provides a method of vaccinating a subject, comprising administering to the subject a composition described herein. In another aspect, the disclosure provides a method of generating an immune response in a subject, comprising administering to the subject a composition described herein. In another aspect, the disclosure provides a method of treating or preventing RSV disease in a subject, comprising administering to the subject a composition described herein. In another aspect, the disclosure provides a composition described herein for use in vaccinating, generating an immune response, or treating or preventing RSV disease. In another aspect, the disclosure provides a method of making a composition described herein, comprising culturing host cells modified to express one or more polypeptides as described herein. In another aspect, the disclosure provides a composition, method, or use as described herein.

Any aspect or embodiment described herein can be combined with any other aspect or embodiment as disclosed herein. Further aspects, embodiments, and advantages of the invention will be apparent from the Detailed Description that follows.

### BRIEF DESCRIPTION OF THE DRAWINGS

These and other features, aspects, and advantages of the present invention will become better understood with regard to the following description, and accompanying drawings, where:

FIG. 1 shows a structural model of RSV F protein in the prefusion conformation (PDB 4MMU), with stabilizing elements separated into five different spaces. Spaces 1-4 were targeted by stabilizing mutations. Space 5 refers to the C terminus of the protein.

FIG. 2 shows a close-up view of the structure of C termini of RSV F protein determined by X-ray crystallography of prefusion RSV F (PDB 4MMU) before and after remodeling. Residues that are remodeled (residues 503-509) are outlined with a thicker black highlight (left) and additional structure added by remodeling is shown in black (right).

FIG. 3 shows ddG scoring with representative designs highlighted.

FIG. 4 shows hydrophobicity scoring of designs. Mean (solid line) and standard deviation (dashed lines), WT (dotted line).

FIG. 5 shows a representative electron micrograph of a protein nanostructure as described herein.

FIG. 6A shows a structural model of a PIV5 F protein before (left) and after (right) remodelling of the C terminus. Omitted or unstructured regions (left, not shown) are predicted to adopt an alpha-helical structure (right, dark black).

FIG. 6B shows a structural model of a PIV3 F protein before (left) and after (right) remodelling of the C terminus.

FIG. 6C shows a structural model of a Nipah F protein before (left) and after (right) remodelling of the C terminus.

FIG. 6D shows a structural model of an hMPV F protein before (left) and after (right) remodelling of the C terminus.

FIG. 6E shows a structural model of a SARS-COV-2 S protein before (left) and after (right) remodelling of the C terminus.

FIG. 7 shows predicted ddG for Paramyxoviridea as a function of remodel length. Topleft: PIV5, topbottom: PIV3, topright: Nipah. Dotted line represents the mean, solid line represents the WT sequence. Note that the WT sequence only includes structured residues present in the PDB.

FIG. 8 shows representative remodeled designs from HMPV using RFdiffusion. De novo regions are colored black, context from the input PDB colored white.

FIG. 9 shows predicted ddG for Pneumoviridae and Coronavirdae as a function of remodel length. Top: HMPV, bottom: SARS-COV-2. Dotted line represents the mean, solid line represents the WT sequence. Note that the WT sequence only includes structured residues present in the PDB.

FIG. 10 shows predicted hydrophobicity for Paramyxoviridea as a function of remodeled sequence position. Topleft: PIV5, topbottom: PIV3, topright: Nipah. Dotted line represents the mean, solid line represents the WT sequence. Note that the WT sequence only includes structured residues present in the PDB.

FIG. 11 shows Predicted hydrophobicity for Pneumoviridae and Coronavirdae as a function of remodeled sequence position. Top: HMPV, bottom: SARS-COV-2. Dotted line represents the mean, solid line represents the WT sequence. Note that the WT sequence only includes structured residues present in the PDB.

FIG. 12 shows Principal Component Analysis of distances in group 1 (parallel) remodeled sequences.

FIG. 13 shows Principal Component Analysis of distances in group 2 (not parallel) remodeled sequences.

FIGS. 14A-14C show position specific probabilities for group 1 (parallel). Probabilities represent the likelihood of remodeled length. FIG. 14A shows position specific probabilities for Clust_p2. FIG. 14B shows position specific probabilities for Clust_p1. FIG. 14C shows position specific probabilities for Clust_p0.

FIGS. 15A-15D show position specific probabilities for group 2 (not parallel). Probabilities represent the likelihood of remodeled length. FIG. 15A shows position specific probabilities for Clust_o0. FIG. 15B shows position specific probabilities for Clust_o1. FIG. 15C shows position specific probabilities for Clust_o3. FIG. 15D shows position specific probabilities for Clust_o2.

FIGS. 16A-16G show positional weightings for each cluster. FIG. 16A shows Positional weightings for Clust_p0. FIG. 16B shows Positional weightings for Clust_p1. FIG. 16C shows Positional weightings for Clust_p2. FIG. 16D shows Positional weightings for Clust_o0. FIG. 16E shows Positional weightings for Clust_o1. FIG. 16F shows Positional weightings for Clust_o2. FIG. 16G shows Positional weightings for Clust_o3.

FIG. 17 shows neutralizing titers against RSV/B (B18537 strain) elicited by various nanostructure immunogens based on RSV/B antigens.

FIG. 18 shows neuralizing titers against RSV/A (Tracy strain) elicited by various nanostructure immunogens based on RSV/A antigens.

FIG. 19A and FIG. 19B show a structural comparison of cryo-EM structures of the RSV F ectodomains of A) RSV/A.023, and B) DS-Cav1 fused to foldon (PDB 7LUE). The added C-terminal alpha-helical segment in RSV/A.023 is colored in dark gray and surrounded by a dashed box. Antibody structures were removed from the model of PDB 7LUE prior to generating images.

FIG. 20B and FIG. 20B show shows a structural comparison of C-terminal regions for cryo-EM structures of the RSV Fectodomains of A) RSV/A.023, and B) DS-Cav1 fused to foldon (PDB 7LUE). The added C-terminal alpha-helical segment in RSV/A.023 is colored in dark gray and surrounded by a dashed box. Antibody structures were removed from the model of PDB 7LUE prior to generating images.

FIG. 21 shows maximum binding to the monoclonal antibody 3×1 by biolayer interferometry to remodeled PIV3 F (Top) and maximum binding normalized to the anti-Component A specific antibody 16A8 maximum binding.

FIG. 22 shows maximum binding to the monoclonal antibody PIA174 by biolayer interferometry to remodeled PIV3 F (Top) and maximum binding normalized to the anti-Component A specific antibody 16A8 maximum binding.

FIG. 23 shows maximum binding to the monoclonal antibody 16A8 by biolayer interferometry.

FIG. 24 shows maximum binding of PIV3 F with generic C-terminal remodel sequences to the monoclonal antibody 16A8 by biolayer interferometry.

FIG. 25 shows maximum binding to the monoclonal antibody 3×1 by biolayer interferometry to PIV3 F with generic C-terminal remodel sequences (Top) and maximum binding normalized to the anti-Component A specific antibody 16A8 maximum binding.

FIG. 26 shows maximum binding to the monoclonal antibody PIA174 by biolayer interferometry to PIV3 F with generic C-terminal remodel sequences (Top) and maximum binding normalized to the anti-Component A specific antibody 16A8.

### DETAILED DESCRIPTION

Before the embodiments of the disclosure are described, it is to be understood that such embodiments are provided by way of example only, and that various alternatives to the embodiments of the disclosure described herein may be employed in practicing the invention. Numerous variations, changes, and substitutions will occur to those skilled in the art and may be practiced without departing from spirit of the invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole.

### I. Definitions

The singular forms “a,” “an,” and “the” are intended to include the plural forms as well, unless the context clearly indicates otherwise.

As used herein, the term “about” means a range of values including the specified value, which a person of ordinary skill in the art would consider reasonably similar to the specified value. For example, about means within a standard deviation using measurements generally acceptable in the art. For example, about means a range extending to +/−10%, +/−5%, +/−3%, or +/−1% of the specified value.

The term “at least” followed by a number is used herein to denote the start of a range beginning with that number (which may be a range having an upper limit or no upper limit, depending on the variable being defined). For example, “at least 1” means 1 or more than 1.

The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number)” this means a range whose lower limit is the first number and whose upper limit is the second number. For example, 25 to 100 mm means a range whose lower limit is 25 mm, and whose upper limit is 100 mm.

The term “identical” or percent “identity,” in the context of two or more nucleic acid or polypeptide sequences, refers to two or more sequences or subsequences that are the same or have a specified percentage of amino acid residues or nucleotides that are the same, when compared and aligned for maximum correspondence. Methods of alignment of sequences for comparison are well known in the art. Once aligned, the number of matches is determined by counting the number of positions where an identical nucleotide or amino acid residue is present in both sequences. The percent sequence identity is determined by dividing the number of matches in the alignment by the length of the reference sequence, followed by multiplying the resulting value by 100. For example, a peptide sequence that has 1166 matches when aligned with a reference sequence having 1554 amino acids is 75.0 percent identical to the test sequence (1166÷1554*100=75.0). As the terms are used herein, gaps in the alignment do not decrease the percent sequence identity. Unless otherwise specified, optimal alignment of sequences for comparison is conducted by the global alignment algorithm of Needleman and Wunsch, Mol. Biol. 48:443 (1970) as implemented by EMBOSS Needle (on the World Wide Web at ebi.ac.uk/Tools/psa/emboss_needle/) (Madeira et al. Nucleic Acids Res. 50 (W1):W276-W279 (2022)). Other alignment methods may be used, including without limitation those described in Devereux, et al, Nucleic Acids Res. 12:387-95 (1984); Atschul et al. J. Mo. Biol. 215:403-10 (1990) (BLAST); Carrillo and Lipman Siam J. Appl. Math. 48 (5) (1988); Computational Molecular Biology (Lesk, A M, ed., 1989); Biocomputing Informatics and Genome Projects, (Smith, DW, ed., 1993); Computer Analysis of Sequence Data, Part I, (Griffin and Griffin, eds., 1994); Sequence Analysis in Molecular Biology (von Heinje, 2012); Sequence Analysis Primer (Gribskov and Devereux, J., eds. 1993). Sequence identity is calculated using the implementation of the Needleman-Wunsch algorithm provided by the National Library of Medicine (on the World Wide Web at blast.ncbi.nlm.nih.gov/Blast.cgi?PAGE_TYPE=BlastSearch&BLAST_SPEC-GlobalAln).

For example, sequence identity can be determined by standard methods that are commonly used to compare the similarity of two polypeptide or two polynucleotide sequences. Using a computer program such as EMBOSS Needle or BLAST, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (a standard scoring matrix; see Dayhoff et al., in Atlas of Protein Sequence and Structure, vol. 5, supp. 3 (1978)) that can be used in conjunction with the computer program.

As used herein, the term “helix-forming segment” refers to a portion of a protein or polypeptide that forms, or is predicted to form, an alpha-helix. An “alpha-helix” is an element of protein secondary structure stabilized by hydrogen bonds between carbonyl oxygen and the amnino group of every third residue in the helical turn. The smallest segment of a protein that is generally considered to form an alpha-helix is about 6-7 amino acid results. Accordingly, in some embodiments, a helix-forming segment comprises between about 5 and about 30 amino acid residues, between about 7 and about 14 amino acid residues, between about 7 and about 21 amino acid residues, between about 7 and about 28 amino acid residues, between about 7 and about 35 amino acid residues, between about 7 and about 42 amino acid residues, or between about 7 and about 49 amino acid residues; or any values therebetween, such as without limitation 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or more amino acids. In some embodiments, the helix forming segment forms a parallel, three-helix bundle.

As used herein the term “alpha-helical homotrimer” refers to a three-helix bundle with helices in parallel orientation. The term excludes six-helical bundles such as those formed by assembly of three anti-parallel, two-helix bundles; i.e., the term “alpha-helical homotrimer” as used herein excludes heptad-repeat regions of gp41 or recombinant variants thereof.

As used herein, the term “stable” such as in “stable alpha-helical homotrimer” means that the protein structure (e.g., homotrimer) persists under suitable conditions. A stable protein structure may be detected by biophysical or biochemical methods known in the art-including but not limited to size exclusion chromotagraphy, dynamic light scattering, electron microscopy, analytical ultracentrifugation, X-ray crystallography, nuclear magnetic resonance spectroscopy, circular dichroism, thermal denaturation, or interaction measurements. A “stable” alpha-helical homotrimer may be distinguished from an unstable homotrimer in part by structural analysis (e.g., by X-ray crystallography, NMR, or EM), or by measuring the impact of the alpha-helical homotrimer, for example by binding studies (BLI, SPR) or biophysical studies (thermal denaturation). In some embodiments, the stable alpha-helical homotrimer may be stable at room temperature and/or at elevated temperatures (e.g., 40° C.). An alpha-helical homotrimer may either form a homotrimer in isolation, or as part of a larger trimeric protein complex (such as a trimeric antigen). In some embodiments, inclusion of the stable alpha-helical homotrimer stabilizes the trimeric protein complex by a ΔΔG of at least −10, at least −20, at least −30, at least −40, at least −50, or at least −60, as predicted computationally or experimentally determined. In some embodiments, the stable alpha-helical homotrimer is an “obligate” homotrimer.

As used here, “conservative amino acid substitution” means that: hydrophobic amino acids (Ala, Gly, Met, Val, Ile, Leu, Phe, Thr, Trp) are substituted with other hydrophobic amino acids; hydrophobic amino acids with bulky side chains (Phe, Tyr, Trp) are substituted with other hydrophobic amino acids with bulky side chains; amino acids with positively charged side chains (Arg, His, Lys) are substituted with other amino acids with positively charged side chains; amino acids with negatively charged side chains (Asp, Glu) are substituted with other amino acids with negatively charged side chains; and polar amino acids (Cys, Ser, Thr, Asn, Gly, Tyr) are substituted with other polar amino acids.

| Amino Acid | Three letter symbol | One letter symbol |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Throughout this specification and the claims which follow, unless the context requires otherwise, the word “comprise”, and variations such as “comprises” and “comprising,” as well as “has” or “having” and “includes” or “including,” will be understood to imply the inclusion of a stated element or step or group of elements or steps but not the exclusion of any other element or step or group of elements or steps. “Consisting essentially of” or “consists essentially” indicates exclusion of elements or steps that materially affect the basic and novel characteristics of the claimed invention.

### II. Engineered Ectodomains

The disclosure provides an engineered ectodomain of trimeric viral proteins, including but not limited to paramyxoviridae, pneuomoviridae, rhabdoviridae, filoviridae, herpesviridae, orthomyxoviridae, coronaviridae, retroviridae, and arenviridae. Table 1 shows viral fusion protein that are designable. In some embodiments, the trimer viral protein is an enveloped viral fusion protein.

TABLE 1

| | | Order | | |
|---|---|---|---|---|
| Indication | Protein | Family | Genus | Class |
| PIV3 | Fusion (F) | Mononegavirales | Respirovirus | I |
| | | Paramyxoviridae | | |
| PIV5 | | Mononegavirales | | I |
| | | Paramyxoviridae | | |
| Nipah | Fusion (F) | Mononegavirales | Henipavirus | I |
| | | Paramyxoviridae | | |
| HMPV | Fusion (F) | Mononegavirales | | I |
| | | Pneumoviridae | | |
| RSV | Fusion (F) | Mononegavirales | | I |
| | | Pneumoviridae | | |
| Hendra | Fusion (F) | Mononegavirales | Henipavirus | I |
| virus | | Paramyxoviridae | | |
| Langya | Fusion (F) | Mononegavirales | Henipavirus | I |
| virus | | Paramyxoviridae | | |
| Measles | Fusion (F) | Mononegavirales | Morbilovirus | I |
| morbilo- | | Paramyxoviridae | | |
| virus | | | | |
| Ebolavirus | glycoprotein (GP) | Mononegavirales | Ebolavirus | I |
| | | Filoviridae | | |
| Newcastle | hemagglutinin- | Mononegavirales | Orthoavula- | I |
| Disease | neuraminidase | Paramyxoviridae | virus | |
| Virus | (HN) | | | |
| Human | Fusion (F) | Mononegavirales | Respirovirus | I |
| respiro- | | Paramyxoviridae | | |
| virus 1 | | | | |
| Human | Fusion (F) | Mononegavirales | Respirovirus | I |
| respiro- | | Paramyxoviridae | | |
| virus 3 | | | | |
| Influenza | hemagglutinin | Articulavirales | | I |
| | (HA) | Orthomyxoviridae | | |
| MERS | Spike (S) | Nidovirales | Betacorona- | I |
| | | Coronaviridae | virus | |
| SARS | Spike (S) | Nidovirales | Betacorona- | I |
| | | Coronaviridae | virus | |
| SARS-2 | Spike (S) | Nidovirales | Betacorona- | I |
| | | Coronaviridae | virus | |
| HIV | evelope | Ortervirales | Lentivirus | |
| | glycoprotein | Retroviridae | | |
| | (gp120) | | | |
| Lassa | glycoprotein (GP) | Bunyavirales | Mammarena- | I |
| | | Arenaviridae | virus | |
| Rabies | Glycoprotein | Mononegavirales | | III |
| | (G)Mononega- | Rhabdoviridae | | |
| | virales | | | |
| hCMV gB | glycoprotein | Herpesvirales | Cytomegalo- | III |
| | B (gB) | Herpesviridae | virus | |
| | Herpesvirales | | | |
| HSV | glycoprotein | Herpesvirales | Simplexvirus | III |
| | B (gB) | Herpesviridae | | |
| | Herpesvirales | | | |

In one aspect, the disclosure provides a recombinant polypeptide, comprising an engineered ectodomain of a trimeric viral protein, wherein the ectodomain comprises a C-terminal helix forming segment comprising one or more amino acid substitutions, relative to a native reference sequence of the viral protein, selected such that the segment forms a alpha-helical homotrimer.

In some embodiments, the C-terminal helix forming segment has improved hydrophobic packing compared to the native reference sequence. In some embodiments, the C-terminal helix forming segment comprises between about 7 and about 31 residues. In some embodiments, the amino acid substitutions comprise polar, charged and/or hydrophobic amino acids. In some embodiments, the C-terminal helix forming segment comprises a polypeptide sequence according to any one of LXXTIXXLLXIXXXLXXXL (SEQ ID NO: 566), LVXTXKXLXDLIXXLXXLLXKLXX (SEQ ID NO: 567), LNKVKKXVXXLXXXVXXLEKXLX (SEQ ID NO: 568), EKIXXAIKKAXKL (SEQ ID NO: 569), EXIXKAIKXLXXXXX (SEQ ID NO: 570), XKXXEXXXXVXXXXXXXXX (SEQ ID NO: 571), XXLKKAAXIXKKXLKXX (SEQ ID NO: 572).

In some embodiments, the C-terminal helix forming segment comprises a polypeptide sequence according to any one of the consensus sequences in Table 24.

In some embodiments, the segment comprises a polypeptide sequence according to any one of L X₂ X₂ T I X₂ X₂ L L X₂ I [V/I] X₂ X₂ L [I/L] X₂ X₂ L (SEQ ID NO: 573), L V [A/T] T X₂ K X₂ L X₂ D L I X₂ X₂ L [K/E] X₂ L L X₂ K L X₂ X₂ (SEQ ID NO: 574), or L N K V K K X₂ V X₂ X₂ L X₂ X₂ X₂ V X₂ X₂ L E K X₂ L X₂ (SEQ ID NO: 575), wherein X₂ is polar and charged residues selected from S, T, N, Q, E, D, R, K, and H, preferably wild type amino acid.

In some embodiments, segment comprises a polypeptide sequence according to any one of E K I X₂ X₂ A I K K A X₂ KL (SEQ ID NO: 576), E X₂ I X₂ K A I K X₂ L [L/X₂] X₂ X₂ [X₁/X₂] X₂ (SEQ ID NO: 577), X₂ K [X₁/T] [L/E] E [T/A] X₁ X₂ [I/X₂] V X₂ X₂ [X₁/X₂] [X₁/X₂] X₂ X₂ X₁ X₂ X₂ (SEQ ID NO: 578), or X₂ X₂ L K K A A X₂ I X₁ K K X₁ L K X₂ X₂ (SEQ ID NO: 579), wherein X₁ is apolar residues selected from A, I, L, and M, and wherein X₂ is polar and charged residues selected from S, T, N, Q, E, D, R, K, and H, preferably wild type amino acid. In some embodiments, the segment comprises a polypeptide sequence listed in Table 25A or Table 25B. In some embodiments, the native reference sequence of the viral protein is any one of SEQ ID NOs: 1, 104, 327, 382, 459, 499.

### Respiratory Syncytial Virus (RSV) F Protein

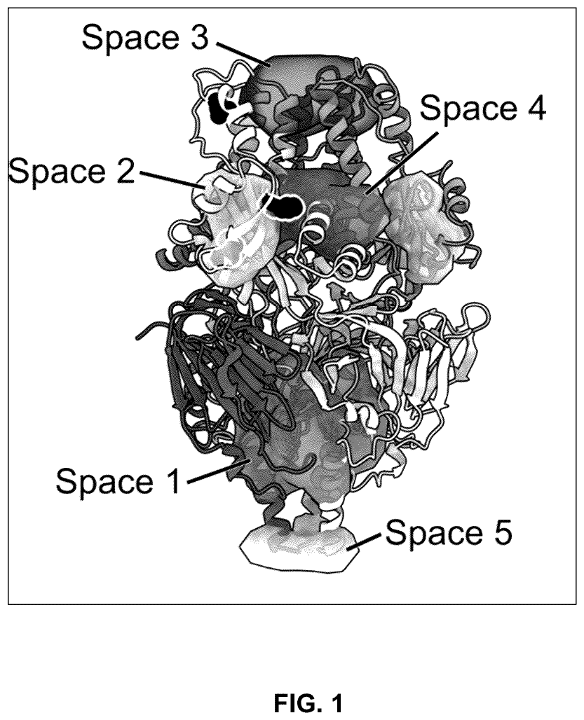

Respiratory Syncytial Virus (RSV) F protein is a major conserved surface antigen of RSV and antibodies against it are associated with protection against disease. RSV F protein is a validated target for protection against infection by RSV as demonstrated by the clinical efficacy of palivizumab, a monoclonal antibody that binds F-antigen and leads to neutralization of the virus (Johnson et al., J Infect Dis. 1997 November; 176 (5): 1215-24). RSV F protein is known to undergo a significant change in structure from prefusion to postfusion form which catalyzes viral and host membrane fusion to allow for viral entry into the cell (Mclellan et al., Science. 2013; 342 (6158): 592-8). Prefusion F protein has important epitopes that are lost during the transition to postfusion F protein (Melero et al., Vaccine. 2017; 35 (3): 461-468). Antibody depletion studies with human sera absorbed with RSV F protein in either conformation demonstrate that the majority of the neutralizing response against RSV F protein targets the prefusion structure (Krarup et al., Nat Commun. 2015; 6:8143). These studies also demonstrate the potential for antibodies that bind postfusion F protein to interfere with neutralization (Ngwuta et al., Sci Transl Med. 2015; 7 (309): 309ra162). In general, high levels of antibodies against RSV F protein are associated with protection against severe disease. However, generating high-titers of neutralizing antibodies against RSV F protein remains challenging, due to the specific biochemical nature of the RSV F protein and the unpredictability of vaccine responses to RSV F. Structural model of RSV F protein in the prefusion conformation is shown in FIG. 1, with stabilizing elements separated into five different spaces. Spaces 1-4 were targeted by stabilizing mutations. Space 5 refers to the C terminus of the protein.

Illustrative sequences are shown in Table 2A. A native RSV/B F protein sequence was used for design (GenBank: WDV37446.1). The (predicted) transmembrane region is residues 527-549 and is bold/underlined. The signal peptide is underlined with italic. The approximate region surrounding the p27 peptide is bold.

TABLE 2A

| | | | SEQ |
|---|---|---|---|
| | | | ID |
| | Description | Sequence | NO: |
| RSV/B | GenBank: | MELLIHRSSAIFLTLAINALYLTSSQNIT | 1 |
| F protein | WDV37446.1 | EEFYQSTCSAVSRGYLSALRTGWYTSVIT | |
| | Reference | IELSNIKETKCNGTDTKVKLIKQELDKYK | |
| | sequence | NAVTELQLLMQNTPAVNNRARREAPQYMN | |
| | | YTINTTKNLNVSISKKRKRRFLGFLLGVG | |
| | | SAIASGIAVSKVLHLEGEVNKIKNALQLT | |
| | | NKAVVSLSNGVSVLTSRVLDLKNYINNQL | |
| | | LPMVNRQSCRISNIETVIEFQQKNSRLLE | |
| | | ITREFSVNAGVTTPLSTYMLTNSELLSLI | |
| | | NDMPITNDQKKLMSSNVQIVRQQSYSIMS | |
| | | IIKEEVLAYVVQLPIYGVIDTPCWKLHTS | |
| | | PLCTTNIKEGSNICLTRTDRGWYCDNAGS | |
| | | VSFFPQADTCKVQSNRVFCDTMNSLTLPS | |
| | | EVSLCNTDIFNSKYDCKIMTSKTDISSSV | |
| | | ITSLGAIVSCYGKTKCTASNKNRGIIKTF | |
| | | SNGCDYVSNKGVDTVSVGNTLYYVNKLEG | |
| | | KNLYVKGEPIINYYDPLVFPSDEFDASIS | |
| | | QVNEKINQSLAFIRRSDELLHNVNTGKST | |
| | | TNIMITAITIVIIVVLLSLIAIGLLLYCK | |
| | | AKNTPVTLSKDQLSGINNIAFSK | |
| RSV/B | GenBank: | MELLIHRSSAIFLTLAINALYLTSSQNIT | 2 |
| F protein | WDV37446.1 | EEFYQSTCSAVSRGYLSALRTGWYTSVIT | |
| | DS-Cav 1 | IELSNIKETKCNGTDTKVKLIKQELDKYK | |
| | (S155C, S290C, | NAVTELQLLMQNTPAVNNRARREAPQYMN | |
| | S190F, V207L) | YTINTTKNLNVSISKKRKRRFLGFLLGVG | |
| | | SAIASGIAVCKVLHLEGEVNKIKNALQLT | |
| | | NKAVVSLSNGVSVLTCRVLDLKNYINNQL | |
| | | LPMLNRQSCRISNIETVIEFQQKNSRLLE | |
| | | ITREFSVNAGVTTPLSTYMLINSELLSLI | |
| | | NDMPITNDQKKLMSSNVQIVRQQSYSIMC | |
| | | IIKEEVLAYVVQLPIYGVIDTPCWKLHTS | |
| | | PLCTTNIKEGSNICLTRTDRGWYCDNAGS | |
| | | VSFFPQADTCKVQSNRVFCDTMNSLTLPS | |
| | | EVSLCNTDIFNSKYDCKIMTSKTDISSSV | |
| | | ITSLGAIVSCYGKTKCTASNKNRGIIKTF | |
| | | SNGCDYVSNKGVDTVSVGNTLYYVNKLEG | |
| | | KNLYVKGEPIINYYDPLVFPSDEFDASIS | |
| | | QVNEKINQSLAFIRRSDELLHNVNTGKST | |
| | | TNIMITAITIVIIVVLLSLIAIGLLLYCK | |
| | | AKNTPVTLSKDQLSGINNIAFSK | |
| RSV/B | Without signal | QNITEEFYQSTCSAVSRGYLSALRTGWYT | 3 |
| F protein | peptide | SVITIELSNIKETKCNGTDTKVKLIKQEL | |
| Ectodomain | | DKYKNAVTELQLLMQNTPAVNNRARREAP | |
| | | QYMNYTINTTKNLNVSISKKRKRRFLGFL | |
| | | LGVGSAIASGIAVSKVLHLEGEVNKIKNA | |
| | | LQLTNKAVVSLSNGVSVLTSRVLDLKNYI | |
| | | NNQLLPMVNRQSCRISNIETVIEFQQKNS | |
| | | RLLEITREFSVNAGVTTPLSTYMLTNSEL | |
| | | LSLINDMPITNDQKKLMSSNVQIVRQQSY | |
| | | SIMSIIKEEVLAYVVQLPIYGVIDTPCWK | |
| | | LHTSPLCTTNIKEGSNICLTRTDRGWYCD | |
| | | NAGSVSFFPQADTCKVQSNRVFCDTMNSL | |
| | | TLPSEVSLCNTDIFNSKYDCKIMTSKTDI | |
| | | SSSVITSLGAIVSCYGKTKCTASNKNRGI | |
| | | IKTFSNGCDYVSNKGVDTVSVGNTLYYVN | |
| | | KLEGKNLYVKGEPIINYYDPLVFPSDEFD | |
| | | ASISQVNEKINQSLAFIRRSDELLHNVNT | |
| | | GKSTTNIMITAITIVIIVVLLSLIAIGLL | |
| | | LYCKAKNTPVTLSKDQLSGINNIAFSK | |
| RSV/B | Without signal | QNITEEFYQSTCSAVSRGYLSALRTGWYT | 4 |
| F protein | peptide | SVITIELSNIKETKCNGTDTKVKLIKQEL | |
| Ectodomain | DS-Cav 1 | DKYKNAVTELQLLMQNTPAVNNRARREAP | |
| | (S155C, S290C, | QYMNYTINTTKNLNVSISKKRKRRFLGFL | |
| | S190F, V207L) | LGVGSAIASGIAVCKVLHLEGEVNKIKNA | |
| | | LQLTNKAVVSLSNGVSVLTCRVLDLKNYI | |
| | | NNQLLPMLNRQSCRISNIETVIEFQQKNS | |
| | | RLLEITREFSVNAGVTTPLSTYMLTNSEL | |
| | | LSLINDMPITNDQKKLMSSNVQIVRQQSY | |
| | | SIMCIIKEEVLAYVVQLPIYGVIDTPCWK | |
| | | LHTSPLCTTNIKEGSNICLTRTDRGWYCD | |
| | | NAGSVSFFPQADTCKVQSNRVFCDTMNSL | |
| | | TLPSEVSLCNTDIFNSKYDCKIMTSKTDI | |
| | | SSSVITSLGAIVSCYGKTKCTASNKNRGI | |
| | | IKTFSNGCDYVSNKGVDTVSVGNTLYYVN | |
| | | KLEGKNLYVKGEPIINYYDPLVFPSDEFD | |
| | | ASISQVNEKINQSLAFIRRSDELLHNVNT | |
| | | GKSTTNIMITAITIVIIVVLLSLIAIGLL | |
| | | LYCKAKNTPVTLSKDQLSGINNIAFSK | |
| RSV/B | Without signal | QNITEEFYQSTCSAVSKGYLSALRTGWYT | 1236 |
| F protein | peptide | SVITIELSNIKENKCNGTDAKVKLIKQEL | |
| Ectodomain | DS-Cav 1 | DKYKNAVTELQLLMQSTPATNNRARRELP | |
| | (S155C, S290C, | RFMNYTLNNAKKTNVTLSKKRKRRFLGFL | |
| | S190F, V207L) | LGVGSAIASGVAVCKVLHLEGEVNKIKSA | |
| | | LLSTNKAVVSLSNGVSVLTFKVLDLKNYI | |
| | | DKQLLPILNKQSCSISNIETVIEFQQKNN | |
| | | RLLEITREFSVNAGVTTPVSTYMLTNSEL | |
| | | LSLINDMPITNDQKKLMSNNVQIVRQQSY | |
| | | SIMCIIKEEVLAYVVQLPLYGVIDTPCWK | |
| | | LHTSPLCTTNTKEGSNICLTRTDRGWYCD | |
| | | NAGSVSFFPQAETCKVQSNRVFCDTMNSL | |
| | | TLPSEVNLCNVDIFNPKYDCKIMTSKTDV | |
| | | SSSVITSLGAIVSCYGKTKCTASNKNRGI | |
| | | IKTFSNGCDYVSNKGVDTVSVGNTLYYVN | |
| | | KQEGKSLYVKGEPIINFYDPLVFPSDEFD | |
| | | ASISQVNEKINQSLAFIRKSDELL | |
| RSV/B | Without signal | QNITEEFYQSTCSAVSRGYFSALRTGWYT | 1237 |
| F protein | peptide | SVITIELSNITETKCNGTDTKVKLIKQEL | |
| Ectodomain | | DKYKNAVTELQLLMQNTPAANNRARREAP | |
| | | QHMNYTINTTKNLNVSISKKRKRRFLGFL | |
| | | LGVGSAIASGIAVSKVLHLEGEVNKIKNA | |
| | | LLSTNKAVVSLSNGVSVLTSKVLDLKNYI | |
| | | NNQLLPIVNQQSCRIFNIETVIEFQQKNS | |
| | | RLLEITREFSVNAGVTTPLSTYMLTNSEL | |
| | | LSLINDMPITNDQKKLMSSNVQIVRQQSY | |
| | | SIMSIIKEEVLAYVVQLPIYGVIDTPCWK | |
| | | LHTSPLCTTNIKEGSNICLTRTDRGWYCD | |
| | | NAGSVSFFPQADTCKVQSNRVFCDTMNSL | |
| | | TLPSEVSLCNTDIFNSKYDCKIMTSKTDI | |
| | | SSSVITSLGAIVSCYGKTKCTASNKNRGI | |
| | | IKTFSNGCDYVSNKGVDTVSVGNTLYYVN | |
| | | KLEGKNLYVKGEPIINYYDPLVFPSDEFD | |
| | | ASISQVNEKINQSLAFIRKSDELL | |
| RSV/B | Without signal | QNITEEFYQSTCSAVSRGYFSALRTGWYT | 1238 |
| F protein | peptide | SVITIELSNITETKCNGTDTKVKLIKQEL | |
| Ectodomain | DS-Cav 1 | DKYKNAVTELQLLMQNTPAANNRARREAP | |
| | (S155C, S290C, | QHMNYTINTTKNLNVSISKKRKRRFLGFL | |
| | S190F, V207L) | LGVGSAIASGIAVCKVLHLEGEVNKIKNA | |
| | Stabilized | LLSTNKAVVSLSNGVSVLTFKVLDLKNYI | |
| | muation | NNQLLPILNQQSCRIFNIETVIEFQQKNS | |
| | | RLLEITREFSVNAGVTTPLSTYMLTNSEL | |
| | | LSLINDMPITNDQKKLMSSNVQIVRQQSY | |
| | | SIMCIIKEEVLAYVVQLPIYGVIDTPCWK | |
| | | LHTSPLCTTNIKEGSNICLTRTDRGWYCD | |
| | | NAGSVSFFPQADTCKVQSNRVFCDTMNSL | |
| | | TLPSEVSLCNTDIFNSKYDCKIMTSKTDI | |
| | | SSSVITSLGAIVSCYGKTKCTASNKNRGI | |
| | | IKTFSNGCDYVSNKGVDTVSVGNTLYYVN | |
| | | KLEGKNLYVKGEPIINYYDPLVFPSDEFD | |
| | | ASISQVNEKINQSLAFIRKSDELL | |
| RSV/A | Without signal | QNITEEFYQSTCSAVSKGYLSALRTGWYT | 5 |
| F protein | peptide | SVITIELSNIKENKCNGTDAKVKLIKQEL | |
| Ectodomain | DS-Cav 1 | DKYKNAVTELQLLMQSTPATNNRARRELP | |
| | (S155C, S290C, | RFMNYTLNNAKKTNVTLSKKRKRRFLGFL | |
| | S190F, V207L) | LGVGSAIASGVAVCKVLHLEGEVNKIKSA | |
| | | LLSTNKAVVSLSNGVSVLTFKVLDLKNYI | |
| | | DKQLLPILNKQSCSISNIETVIEFQQKNN | |
| | | RLLEITREFSVNAGVTTPVSTYMLTNSEL | |
| | | LSLINDMPITNDQKKLMSNNVQIVRQQSY | |
| | | SIMCIIKEEVLAYVVQLPLYGVIDTPCWK | |
| | | LHTSPLCTTNTKEGSNICLTRTDRGWYCD | |
| | | NAGSVSFFPQAETCKVQSNRVFCDTMNSL | |
| | | TLPSEVNLCNVDIFNPKYDCKIMTSKTDV | |
| | | SSSVITSLGAIVSCYGKTKCTASNKNRGI | |
| | | IKTFSNGCDYVSNKGVDTVSVGNTLYYVN | |
| | | KQEGKSLYVKGEPIINFYDPLVFPSDEFD | |
| | | ASISQVNEKINQSLAFIRKSDELL | |
| RSV/A2 | GenBank GI: | MELLILKANAITTILTAVTFCFASGQNIT | 1239 |
| F protein | 138251 | EEFYQSTCSAVSKGYLSALRTGWYTSVIT | |
| | Swiss Prot | IELSNIKENKCNGTDAKVKLIKQELDKYK | |
| | P03420 | NAVTELQLLMQSTPPTNNRARRELPRFMN | |
| | | YTLNNAKKTNVTLSKKRKRRFLGFLLGVG | |
| | | SAIASGVAVSKVLHLEGEVNKIKSALLST | |
| | | NKAVVSLSNGVSVLTSKVLDLKNYIDKQL | |
| | | LPIVNKQSCSISNIETVIEFQQKNNRLLE | |
| | | ITREFSVNAGVTTPVSTYMLTNSELLSLI | |
| | | NDMPITNDQKKLMSNNVQIVRQQSYSIMS | |
| | | IIKEEVLAYVVQLPLYGVIDTPCWKLHTS | |
| | | PLCTTNTKEGSNICLTRTDRGWYCDNAGS | |
| | | VSFFPQAETCKVQSNRVFCDTMNSLTLPS | |
| | | EINLCNVDIFNPKYDCKIMTSKTDVSSSV | |
| | | ITSLGAIVSCYGKTKCTASNKNRGIIKTF | |
| | | SNGCDYVSNKGMDTVSVGNTLYYVNKQEG | |
| | | KSLYVKGEPIINFYDPLVFPSDEFDASIS | |
| | | QVNEKINQSLAFIRKSDELLHNVNAGKST | |
| | | TNIMITTIIIVIIVILLSLIAVGLLLYCK | |
| | | ARSTPVTLSKDQLSGINNIAFSN | |
| RSV/B | 18537 strain | MELLIHRSSAIFLTLAVNALYLTSSQNIT | 1240 |
| F protein | GenBank GI: | EEFYQSTCSAVSRGYFSALRTGWYTSVIT | |
| | 138250 | IELSNIKETKCNGTDTKVKLIKQELDKYK | |
| | Swiss Prot | NAVTELQLLMQNTPAANNRARREAPQYMN | |
| | P13843 | YTINTTKNLNVSISKKRKRRFLGFLLGVG | |
| | | SAIASGIAVSKVLHLEGEVNKIKNALLST | |
| | | NKAVVSLSNGVSVLTSKVLDLKNYINNRL | |
| | | LPIVNQQSCRISNIETVIEFQQMNSRLLE | |
| | | ITREFSVNAGVTTPLSTYMLTNSELLSLI | |
| | | NDMPITNDQKKLMSSNVQIVRQQSYSIMS | |
| | | IIKEEVLAYVVQLPIYGVIDTPCWKLHTS | |
| | | PLCTTNIKEGSNICLTRTDRGWYCDNAGS | |
| | | VSFFPQADTCKVQSNRVFCDTMNSLTLPS | |
| | | EVSLCNTDIFNSKYDCKIMTSKTDISSSV | |
| | | ITSLGAIVSCYGKTKCTASNKNRGIIKTF | |
| | | SNGCDYVSNKGVDTVSVGNTLYYVNKLEG | |
| | | KNLYVKGEPIINYYDPLVFPSDEFDASIS | |
| | | QVNEKINQSLAFIRRSDELLHNVNTGKST | |
| | | INIMITTIIIVIIVVLLSLIAIGLLLYCK | |
| | | AKNTPVTLSKDQLSGINNIAFSK | |
| RSV F protein | | MELLILKANAITTILTAVTFCFASGQNIT | 1241 |
| | | EEFYQSTCSAVSKGYLSALRTGWYTSVIT | |
| | | IELSNIKENKCNGTDAKVKLIKQELDKYK | |
| | | NAVTELQLLMQSTPATNNRARRELPRFMN | |
| | | YTLNNAKKTNVTLSKKRKRRFLGFLLGVG | |
| | | SAIASGVAVCKVLHLEGEVNKIKSALLST | |
| | | NKAVVSLSNGVSVLTFKVLDLKNYIDKQL | |
| | | LPILNKQSCSISNIETVIEFQQKNNRLLE | |
| | | ITREFSVNAGVTTPVSTYMLTNSELLSLI | |
| | | NDMPITNDQKKLMSNNVQIVRQQSYSIMC | |
| | | IIKEEVLAYVVQLPLYGVIDTPCWKLHTS | |
| | | PLCTTNTKEGSNICLTRTDRGWYCDNAGS | |
| | | VSFFPQAETCKVQSNRVFCDTMNSLTLPS | |
| | | EVNLCNVDIFNPKYDCKIMTSKTDVSSSV | |
| | | ITSLGAIVSCYGKTKCTASNKNRGIIKTF | |
| | | SNGCDYVSNKGVDTVSVGNTLYYVNKQEG | |
| | | KSLYVKGEPIINFYDPLVFPSDEFDASIS | |
| | | QVNEKINQSLAFIRKSDELLSAIGGYIPE | |
| | | APRDGQAYVRKDGEWVLLSTEL | |

In some embodiments, the RSV refers RSV/A. In some embodiments, the RSV refers RSV/B.

In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 1. In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 2. In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 3. In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 4. In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 5.

In another aspect, the disclosure provides a recombinant polypeptide, comprising an engineered ectodomain of a Respiratory Syncytial Virus (RSV) fusion (F) protein, wherein the ectodomain comprises: (a) one, two, three or more amino acid substitutions at positions 140, 399, 400, 485, 486, 487, 488, 489, 494, or 498 relative to SEQ ID NO: 1, (b) one, two, three or more amino acid substitutions at positions 56, 58, 154, 187, 296, or 298 relative to SEQ ID NO: 1, (c) one, two, three or more amino acid substitutions at positions 75, 216, 218, or 219 relative to SEQ ID NO: 1, (d) one, two, three or more amino acid substitutions at positions 92, 232, 235, 238, 249, 250, or 254 relative to SEQ ID NO: 1, (e) one, two, three or more amino acid substitutions at positions 67, 137, or 339 relative to SEQ ID NO: 1, (f) a substitution of a non-cleavable linker in place of a furin cleavage site at about residue 100 to about residue 140 relative to SEQ ID NO: 1, or (g) any combination of (a)-(f).

### C-Terminal Helix-Forming Segment

The C-terminal end of the ectodomain of many viral fusion proteins is, in at least some cases, known to be or predicted to be a helical bundle that interfaces with a helical transmembrane domain. The present inventors have observed that, in the RSV F protein, the C-terminal helical region of the ectodomain has suboptimal hydrophobic packing. Computational modeling (with RosettaRemodel) was used to generate artificial polypeptide sequences, each predicted to form a stable alpha helix. In illustrative, non-limiting Examples provided below, the helical backbone is first optimized with side-chains represented as centroids, and then the side-chains are designed in all-atom mode. Optimal linker length can be determined by a plot of ddG as a function of linker length (Rosetta remodel), or ddG normalized to linker length (RFdiffusion). Then 6-14 additional amino acids were modeled with helical constraints.

Illustrative sequences are shown in Table 2B. Residues 500-502 of the native RSV F protein are included as NOS (bold underline) and are, in these embodiments, conserved with the native sequence whereas many of the other amino acid residues are modified.

TABLE 2B
C-terminal Alpha-helical segments (Rosetta remodel)

| | | Remodeled | |
|---|---|---|---|
| Name | Sequence | Length | SEQ ID NO: |
| C-Term 1 | NQSREIIRAINIVRKIASEK | 17 | 10 |
| C-Term 2 | NQSALWLEAAKYVKQAREKS | 17 | 11 |
| C-Term 3 | NQSAKNAEAAKIAEETKRKD | 17 | 12 |
| C-Term 4 | NQSRETAKAVSAVK | 11 | 75 |
| C-Term 5 | NQSALLLEAAKYVKKAREKS | 17 | 119 |
| C-Term 6 | NQSRKLLEAAEEMEKMLKTS | 17 | 120 |
| C-Term 7 | NQSRKMLEAVEHAKKLKKES | 17 | 121 |
| C-Term 8 | NQSRKMLEAVEKAKKLDKES | 17 | 122 |
| C-Term 9 | NQSAKTEEAYQRTIKTQQKL | 17 | 123 |
| C-Term 10 | NQSRDLDTAAKQVKEMLKEKS | 18 | 124 |
| C-Term 11 | NQSRETEKTIRQVQEILKKWS | 18 | 125 |
| C-Term 12 | NQSREVKEAIKIIKKILKKQS | 18 | 126 |
| C-Term 13 | NQSREIKDAIKKAKEFIKTIK | 18 | 127 |
| C-Term 14 | NQSREIETAIKKAKEFIKTIK | 18 | 128 |
| C-Term 15 | NQSRKATETIKKFEESEKS | 16 | 129 |
| C-Term 16 | NQSRDTIKVAIIVKELYKKIS | 18 | 130 |
| C-Term 17 | NQSRKTLETIEWVKKVIKKQRS | 19 | 131 |
| C-Term 18 | NQSRKTLETIEWVEKVIKKQRS | 19 | 132 |
| C-Term 19 | NQSRKWNESSKKVQEQDS | 15 | 133 |
| C-Term 20 | NQSRKTEKAIRLVLKWLKES | 17 | 134 |
| C-Term 21 | NQSRDTLKAIEQTKRYLEELKKS | 20 | 135 |
| C-Term 22 | NQSRSWDIAAKFVKTVLSNQS | 18 | 136 |
| C-Term 23 | NQSRKTLEATEIAKKLAEDRS | 18 | 137 |
| C-Term 24 | NQSLEILKAAKEAKKLIEDLRRS | 20 | 138 |
| C-Term 25 | NQSKELLDAAKAVKKMLEKEKSS | 20 | 139 |
| C-Term 26 | NQSKKLLDAADAVKKMLEKEKSS | 20 | 140 |
| C-Term 27 | NQSKKVLETIRWIETVISRQRSS | 20 | 141 |
| C-Term 28 | NQSADLKKVAELVKKLMEEAKKKS | 21 | 142 |
| C-Term 29 | NQSTDTMKAARIMKEELKEKS | 18 | 143 |
| C-Term 30 | NQSRKTEEALRRADTIIKQLASKS | 21 | 144 |
| C-Term 31 | NQSKKLKSAADDVKKAKEKS | 17 | 145 |
| C-Term 32 | NQSKELKSAAEDVKKAKEKS | 17 | 146 |
| C-Term 33 | NQSRETKKATENVKTMLTKSKS | 19 | 147 |
| C-Term 34 | NQSLELKKAAKAANTDLTKKS | 18 | 148 |
| C-Term 35 | NQSLELKEAAKAANTDLTKKS | 18 | 149 |
| C-Term 36 | NQSRKLEEIARIVEQKKRTEEKRS | 21 | 150 |
| C-Term 37 | NQSAETKKAIERAREL | 13 | 151 |
| C-Term 38 | NQSRDLKKAAEIAKKS | 13 | 152 |
| C-Term 39 | NQSRTLLETAEIVTRS | 13 | 153 |
| C-Term 40 | NQSRTLLETAEIVKRS | 13 | 154 |
| C-Term 41 | NQSRKLDKAAEYVEKS | 13 | 155 |
| C-Term 42 | NQSKEAKKAIETAKKLS | 14 | 156 |
| C-Term 43 | NQSRKLETAAEKLKQTE | 14 | 157 |
| C-Term 44 | NQSRLMLEAVKIAQSQS | 14 | 158 |
| C-Term 45 | NQSRETKEAAESVKQMES | 15 | 159 |
| C-Term 46 | NQSRRTLKAIEITLKLLS | 15 | 160 |
| C-Term 47 | NQSRRTLTAITRVERKDS | 15 | 161 |
| C-Term 48 | NQSKKLADAADWVETVKSS | 16 | 162 |
| C-Term 49 | NQSKKTHSAIEWVERLVSS | 16 | 163 |
| C-Term 50 | NQSADTKKAAEIAKKLAKS | 16 | 164 |

In some embodiments, modeling suggests that the following substitutions will stabilize the portion of the F protein in a helical conformation.

Illustrative sequences generated by RFdiffusion are shown in Table 2C. Residues 500-502 of the native RSV F protein are included as NQS (bold underline) and are, in these embodiments, conserved with the native sequence whereas many of the other amino acid residues are modified

TABLE 2C
C-terminal Alpha-helical segments for RSV (RFdiffusion)

| | | Remodeled | SEQ ID |
|---|---|---|---|
| Name | Sequence | Length | NO: |
| C-Term 1 | NQSQSIQATTSRVDAIEAKVKHLEA | 23 | 165 |
| C-Term 2 | NQSVTINNMISSNTNEISSLQDRVKHIEDTLA | 31 | 166 |
| | L | | |
| C-Term 3 | NQSKLVKKVIKETHEIKKKLEDLLK | 23 | 167 |
| C-Term 4 | NQSRSNKKTKNKVKSIEKQVKEIEKRLEKLER | 31 | 168 |
| | A | | |
| C-Term 5 | NQSQAIRETQDEVKNLNKRINKIVTSI | 25 | 169 |
| C-Term 6 | NQSRAIKETQKRTTVLEEDLKRVKELLKS | 27 | 170 |
| C-Term 7 | NQSRQIVEVMKEVEELRKRVENIEKNL | 25 | 171 |
| C-Term 8 | NQSQKTRATEEALKKTQKEVTKLKKEIQKLT | 29 | 172 |
| C-Term 9 | NQSRSNKKTKNKVKSIEKQVKEIEKRLEKLEK | 31 | 173 |
| | A | | |
| C-Term 10 | NQSNTVRKTIETVNSLEKELKELRTEVDRLL | 29 | 174 |
| C-Term 11 | NQSKEIRNTVKKVRTIEKRLNKLETSL | 25 | 175 |
| C-Term 12 | NQSRTLKDTTELTKNLNKKLKKLEEEL | 25 | 176 |
| C-Term 13 | NQSKYISNRIKENTDQIKKLEERVTELEA | 27 | 177 |
| C-Term 14 | NQSLEIRQTSKRVESLERRVTQVERDR | 25 | 178 |

TABLE 2D
Possible substitutions at Positions 503-532 (RFdiffusion)

| Position | Preferred | Allowed residues | SEQ ID NO: |
|---|---|---|---|
| L503 | Polar | QVKRNL | 580 |
| A504 | Polar | STLAQKEY | 581 |
| F505 | Hydrophobic | IVNTL | 582 |
| I506 | Polar | QNKRVS | 583 |
| R507 | Polar | ANKEDQ | 584 |
| K508 | Hydrophobic | TMVR | 585 |
| S509 | Hydrophobic | TIKQMEVS | 586 |
| D510 | Polar | SKNDE | 587 |
| E511 | Polar | RSEKATL | 588 |
| L512 | Hydrophobic | VNTL | 589 |
| L513 | Polar | DTHKENR | 590 |
| H514 | Polar | ANESVKTD | 591 |
| N515 | Hydrophobic | IELTQ | 592 |
| V516 | Polar | EIKNRQ | 593 |
| N517 | Polar | ASKER | 594 |
| A518 | Polar | KSQRDE | 595 |
| G519 | Hydrophobic | VLI | 596 |
| I520 | Polar | KQENT | 597 |
| P521 | Polar | HDEKRNQ | 598 |
| E522 | Hydrophobic | LRIV | 599 |
| A523 | Polar | EVLKR | 600 |
| P524 | Polar | AKTER | 601 |
| R525 | Polar | HRSLNED | 602 |
| D526 | Hydrophobic | ILVR | 603 |
| G527 | Polar | EKQD | 604 |
| Q528 | Polar | DKSRA | 605 |
| A529 | Hydrophobic | TL | 606 |
| Y530 | Polar | LET | 607 |
| V531 | Polar | ARK | 608 |
| R532 | Hydrophobic | LA | 609 |

In some embodiments, the C-terminal helix-forming segment comprises between about 10 and about 30 residues. In some embodiments, the segment comprises substitutions relative to the reference sequence SEQ ID NO: 1 at two or more, three or more, or four or more residues that, without being bound by theory, may generate hydrophobic contacts between the segments in the alpha-helical homotrimer.

The computational design described herein has detailed yield information on desirable amino acid substitutions that, individually or in groups, may stabilize the RSV F protein ectodomain. Illustrative, non-limiting amino acid substitutions that may be used are described as follows. In some embodiments, the C-terminal helix-forming segment (“the segment”) comprises amino acid substitutions at one or more of positions 505-519 according to reference SEQ ID NO: 1. It will be readily understood by those skilled in the art that alignment to the reference sequence of this segment depends on preserving the helical structure of the segment, and therefore insertions and deletions in the alignment are not permitted in generating sequence alignment for this segment. The starting amino acid (e.g., F in F505) is included here for clarity only, it being understood that the modification provided herein may be used with other strains of RSV in which the starting amino acid is different from the amino acid in the RSV/B reference strain sequence SEQ ID NO: 1.

In some embodiments, the segment comprises a polypeptide sequence listed in Table 2B, or a polypeptide sequence having between 1 and 5 amino acid substitutions thereto. In some embodiments, the segment comprises a polypeptide sequence listed in Table 2B or having 1, 2, 3, 4, 5, or more amino acid substitutions thereto. In some embodiments, the segment comprises a polypeptide sequence listed in Table 2C, or a polypeptide sequence having between 1 and 5 amino acid substitutions thereto. In some embodiments, the segment comprises polypeptide sequence listed in Table 2C or having 1, 2, 3, 4, 5, or more amino acid substitutions thereto.

In some embodiments, the segment comprises the polypeptide sequence NQSREIIRAINIVRKIASEK (SEQ ID NO: 10), NQSALWLEAAKYVKQAREKS (SEQ ID NO: 11), NQSAKNAEAAKIAEETKRKD (SEQ ID NO: 12), or NQSRETAKAVSAVK (SEQ ID NO: 75), or a polypeptide sequence having between 1 and 5 amino acid substitutions thereto. In some embodiments, the segment comprises the polypeptide sequence NQSREIIRAINIVRKIASEK (SEQ ID NO: 10) or a polypeptide sequence having between 1 and 5 amino acid substitutions thereto. In some embodiments, the segment comprises the polypeptide sequence NQSREIIRAINIVRKIASEK (SEQ ID NO: 10).

In some embodiments, the C-terminal helix-forming segment comprises between about 5 and about 30 residues. In some embodiments, the C-terminal helix-forming segment comprises between about 5 and about 25 residues. In some embodiments, the C-terminal helix-forming segment comprises between about 5 and about 20 residues. In some embodiments, the C-terminal helix-forming segment comprises between about 5 and about 15 residues. In some embodiments, the C-terminal helix-forming segment comprises between about 5 and about 10 residues. In some embodiments, the C-terminal helix-forming segment comprises between about 10 and about 30 residues. In some embodiments, the C-terminal helix-forming segment comprises between about 10 and about 25 residues. In some embodiments, the C-terminal helix-forming segment comprises between about 10 and about 20 residues. In some embodiments, the C-terminal helix-forming segment comprises between about 10 and about 15 residues. In some embodiments, the C-terminal helix-forming segment comprises between about 15 and about 30 residues. In some embodiments, the C-terminal helix-forming segment comprises between about 15 and about 25 residues. In some embodiments, the C-terminal helix-forming segment comprises between about 15 and about 20 residues.

In another aspect, the disclosure provides an alpha-helical segment, comprising a polypeptide sequence listed in Table 25A or Table 25B, or a polypeptide sequence having between 1 and 5 amino acid substitutions thereto. In some embodiments, the polypeptide comprises a trimeric pathogen protein, N-terminally or C-terminally linked to the alpha-helical segment. In some embodiments, the C-terminal helix-forming segment comprises at least 5 residues. In some embodiments, the C-terminal helix-forming segment comprises at least 10 residues. In some embodiments, the C-terminal helix-forming segment comprises at least 15 residues. In some embodiments, the C-terminal helix-forming segment comprises at least 20 residues. In some embodiments, the C-terminal helix-forming segment comprises at least 25 residues.

### Stabilizing Substitutions

Computational modeling was used to identify amino acid substitutions to stabilize RSV/B F protein in the prefusion conformation. Without being bound by theory, the following amino acid substitutions are described herein as “stabilizing substitutions” because they are predicted to stabilize the RSV F protein by increasing shape complementarity within the tertiary structure of RSV F protein in the prefusion conformation. The amino acid substitutions may have other effects on structure, such as generating hydrophobic or charge-charge interactions (e.g., salt bridges) within the structure. These mutations are listed in Table 3A.

TABLE 3A
stabilizing substitutions

| Space | Substitutions |
|---|---|
| Space 1 | F140W, K399A, K399V, T400D, S485I, S485A, |
| | S485F, D486A, D486Q, D486E, D486S, E487R, |
| | E487K, E487A, E487M, E487Q, 487R, 487M, |
| | F488W, D489A, Q494I, Q494M, Q494L, Q494A, |
| | K498A, K498E, 498A, 498Y |
| Space 2 | V56L, V56A, T58A, T58S, T58M, V154I, V187L, |
| | V296A, A298M, A298L, A298I |
| Space 3 | K75Q, N216S, N216D, E218P, T219S |
| Space 4 | E92I, E92A, E232A, E232W, R235Y, R235W, |
| | S238A, S238L, T249P, Y250F, N254V, N254L |
| Other | T67V, F137D, F137S, R339E |

Embodiments of combinations of substitutions are shown in Table 3B.

TABLE 3B

E487R + K498A
E487R + K498E
E487K + K498E
D486A + E487R + K498A
D486Q + E487R + K498A
D486E + E487A + D489A + T400D
D486A + E487M + K498A
E487Q
D486S
F488W + D489A + T400D + E487R + K498A
F140W + D489A + T400D + E487R + K498A
Q494I + S485I + K399A + 487R + 498A
Q494M + S485I + K399A, D486A + 487M + 498A
Q494L + S485A + K399V + D486A + 487M + 498A
Q494M + S485A + K399V + D486A + 487M + 498A
Q494A + S485F + K399V + D486A + 487M + 498Y
D489A + T400D + E487R + K498A
D489A + T400D

In some embodiments, the ectodomain comprises the amino acid substitutions S155C, S290C, S190F, and V207L.

In some embodiments, the ectodomain comprises one, two, three or more amino acid substitutions at positions 140, 399, 400, 485, 486, 487, 488, 489, 494, or 498 relative to SEQ ID NO: 1.

In some embodiments, the ectodomain comprises one or more of the following sets of amino acid substitutions relative to SEQ ID NO: 1: E487R+K498A; E487R+K498E; E487K+K498E; D486A+E487R+K498A; D486Q+E487R+K498A; D486E+E487A+D489A+T400D; D486A+E487M+K498A; E487Q; D486S; F488W+D489A+T400D+E487R+K498A; F140W+D489A+T400D+E487R+K498A; Q494I+S485I+K399A+487R+498A; Q494M+S485I+K399A; D486A+487M+498A; Q494L+S485A+K399V+D486A+487M+498A; Q494M+S485A+K399V+D486A+487M+498A; Q494A+S485F+K399V+D486A+487M+498Y; D489A+T400D+E487R+K498A; or D489A+T400D. In some embodiments, the ectodomain comprises the amino acid substitutions F488W, D489A, T400D, E487R, K498A, and D486A. In some embodiments, the ectodomain comprises the amino acid substitutions F488W, D489A, T400D, E487R, K498A, and T249P.

### Additional Substitutions to Stabilize the F Protein in a Prefusion Conformation

Without being bound by theory, the following amino acid substitutions are predicted to stabilize the RSV F protein. The amino acid substitutions may have other effects on structure, such as generating hydrophobic or charge-charge interactions (e.g., salt bridges) within the structure. These mutations are listed in Table 4A.

TABLE 4A
Substitutions

T54H, S55C, T58M, K66E, N67I, T67I, T67V, N88C,
E92C, E92D, Q98C, Q101P, T103C, R106C, F140W,
L142C, V144C, I148C, A149C, V154I, S155C, L188C,
S190I, S215P, E232A, R235Y, S238C, T249P, N254C,
Q279C, V296A, V296I, A298L, Q361C, N371C, K399A,
T400D, N428C, Y458C, S485I, D486A, D486S, D486N,
E487M, E487Q, E487R, F488W, D489A, D489S, Q494M,
V495Y, K498A

In some embodiments, the ectodomain comprises one, two, three or more amino acid substitutions at positions 54, 55, 58, 66, 67, 88, 92, 98, 101, 103, 106, 140, 142, 144, 148, 149, 154, 155, 188, 190, 207, 215, 232, 235, 238, 249, 254, 279, 290, 296, 298, 361, 371, 399, 400, 428, 458, 485, 486, 487, 488, 489, 494, 495, or 498 relative to SEQ ID NO: 1. In some embodiments, the ectodomain comprises one, two, three or more amino acid substitutions at T54H, S55C, T58M, K66E, N67I, T67I, T67V, N88C, E92C, E92D, Q98C, Q101P, T103C, R106C, F140W, L142C, V144C, I148C, A149C, V154I, S155C, L188C, S190I, S215P, E232A, R235Y, S238C, T249P, N254C, Q279C, V296A, V296I, A298L, Q361C, N371C, K399A, T400D, N428C, Y458C, S485I, D486A, D486S, D486N, E487M, E487Q, E487R, F488W, D489A, D489S, Q494M, V495Y, or K498A relative to SEQ ID NO: 1.

Combinations of substitutions are shown in Table 4B.

TABLE 4B

S155C + S290C + S190F + V207L
S55C + L188C + L142C + N371C + T54H + V296I
S55C + L188C + D486S
S55C + L188C + T54H + S190I
T103C + I148C + S190I + D486S
T103C + I148C + T54H + S190I + V296I + D486S
S55C + L188C + T54H + D486S
S55C + L188C + S190I + D486S
S55C + L188C + T54H + S190I + D486S
S155C + S290C + S190I + D486S
S55C + L188C + L142C + N371C T54H + V296I +
D486S + E487Q + D498S
S155C + S290C + T54H + S190I + V296I

In some embodiments, the ectodomain comprises the amino acid substitutions at S155C, S290C, S190F, and V207L relative to SEQ ID NO: 1. In some embodiments, the ectodomain comprises the amino acid substitutions at S55C, L188C, L142C, N371C, T54H, and V296I relative to SEQ ID NO: 1. In some embodiments, the ectodomain comprises the amino acid substitutions at S55C, L188C, and D486S relative to SEQ ID NO: 1. In some embodiments, the ectodomain comprises the amino acid substitutions at S55C, L188C, T54H, and S190I relative to SEQ ID NO: 1. In some embodiments, the ectodomain comprises the amino acid substitutions at T103C, 1148C, S190I, and D486S relative to SEQ ID NO: 1. In some embodiments, the ectodomain comprises the amino acid substitutions at T103C, 1148C, T54H, S190I, V296I, and D486S relative to SEQ ID NO: 1. In some embodiments, the ectodomain comprises the amino acid substitutions at S55C, L188C, T54H, and D486S relative to SEQ ID NO: 1. In some embodiments, the ectodomain comprises the amino acid substitutions at S55C, L188C, S190I, and D486S relative to SEQ ID NO: 1. In some embodiments, the ectodomain comprises the amino acid substitutions at S55C, L188C, T54H, S190I, and D486S relative to SEQ ID NO: 1. In some embodiments, the ectodomain comprises the amino acid substitutions at S155C, S290C, S190I, and D486S relative to SEQ ID NO: 1. In some embodiments, the ectodomain comprises the amino acid substitutions at S55C, L188C, L142C, N371C T54H, V296I, D486S, E487Q, and D498S relative to SEQ ID NO: 1. In some embodiments, the ectodomain comprises the amino acid substitutions at S155C, S290C, T54H, S190I, and V296I relative to SEQ ID NO: 1.

In some embodiments, a RSV F protein mutant comprises a disulfide mutation selected from the group consisting of 55C and 188C; 155C and 290C; 103C and 148C; and 142C and 371C, such as S55C and L188C, S155C and S290C, T103C and I148C, or L142C and N371C. Examples of pairs of such mutations include: 508C and 509C; 515C and 516C; 522C and 523C, such as K508C and S509C, N515C and V516C, or T522C and T523C.

In some embodiments, a RSV F protein mutant comprises one or more cavity filling mutations selected from the groups shown in Table 4C.

TABLE 4C
Disulfide mutations

| | |
|---|---|
| Amino acid position | Substituted with |

| | | |
|---|---|---|
| S | 55, 62, 155, 190, 290 | I, Y, L, H, M |
| T | 54, 58, 189, 397 | I, Y, L, H, M |
| G | 151 | A, H |
| A | 147, 298 | I, L, H, M |
| V | 164, 187, 192, 207, 220, 296, | I, Y, H |
| | 300, 495 | |
| R | 106 | W |

In some embodiments, a RSV F protein mutant comprises at least one cavity filling mutation selected from the group consisting of: T54H, S190I, and V296I.

In some embodiments, a RSV F protein mutant comprises at least one electrostatic mutation selected from the groups shown in Table 4D.

TABLE 4D
Electrostatic mutations

| | |
|---|---|
| Amino acid position | Substituted with |

| | | |
|---|---|---|
| E | 82, 92, 487 | D, F, Q, T, S, L, H |
| K | 315, 394, 399 | F, M, R, S, L, I, Q, T |
| D | 392, 486, 489 | H, S, N, T, P |
| R | 106, 339 | F, Q, N, W |

In some embodiments, the RSV F protein mutant comprises mutation D486S.

Combinations of substitutions are shown in Table 4E.

TABLE 4E

T103C + I148C + S190I + D486S
T54H + S55C + L188C + D486S
T54H + T103C + I148C + S190I + V296I + D486S
T54H + S55C + L142C + L188C + V296I + N371C
S55C + L188C + D486S
T54H + S55C + L188C + S190I
S55C + L188C + S190I + D486S
T54H + S55C + L188C + S190I + D486S
S155C + S190I + S290C + D486S
T54H + S55C + L142C + L188C + V296I + N371C +
D486S + E487Q + D489S
T54H + S155C + S190I + S290C + V296I
N67I + S215P
N67I + S215P + E487Q
V56C + V164C
I57C + S190C
T58C + V164C
N165C + V296C
K168C + V296C
M396C + F483C

In some embodiments, the ectodomain comprises the amino acid substitutions at T103C, I148C, S190I and D486S relative to SEQ ID NO: 1. In some embodiments, the ectodomain comprises the amino acid substitutions at T54H, S55C, L188C and D486S relative to SEQ ID NO: 1. In some embodiments, the ectodomain comprises the amino acid substitutions at T54H, T103C, I148C, S190I, V296I and D486S relative to SEQ ID NO: 1. In some embodiments, the ectodomain comprises the amino acid substitutions at T54H, S55C, L142C, L188C, V296I and N371C relative to SEQ ID NO: 1. In some embodiments, the ectodomain comprises the amino acid substitutions at S55C, L188C and D486S relative to SEQ ID NO: 1. In some embodiments, the ectodomain comprises the amino acid substitutions at T54H, S55C, L188C and S190I relative to SEQ ID NO: 1. In some embodiments, the ectodomain comprises the amino acid substitutions at S55C, L188C, S190I and D486S relative to SEQ ID NO: 1. In some embodiments, the ectodomain comprises the amino acid substitutions at T54H, S55C, L142C, L188C, V296I, N371C, D486S, E487Q and D489S relative to SEQ ID NO: 1. In some embodiments, the ectodomain comprises the amino acid substitutions at T54H, S155C, S190I, S290C and V296I relative to SEQ ID NO: 1. In some embodiments, the ectodomain comprises the amino acid substitutions at N67I and S215P relative to SEQ ID NO: 1. In some embodiments, the ectodomain comprises the amino acid substitutions at N67I, S215P and E487Q relative to SEQ ID NO: 1. In some embodiments, the ectodomain comprises the amino acid substitutions at V56C and V164C relative to SEQ ID NO: 1. In some embodiments, the ectodomain comprises the amino acid substitutions at 157C and S190C relative to SEQ ID NO: 1. In some embodiments, the ectodomain comprises the amino acid substitutions at T58C and V164C relative to SEQ ID NO: 1. In some embodiments, the ectodomain comprises the amino acid substitutions at N165C and V296C relative to SEQ ID NO: 1. In some embodiments, the ectodomain comprises the amino acid substitutions at K168C and V296C relative to SEQ ID NO: 1. In some embodiments, the ectodomain comprises the amino acid substitutions at M396C and F483C relative to SEQ ID NO: 1.

### Combination of C-Terminal Helix-Forming Segment and Stabilizing Substitutions

In some embodiments, the disclosure provides recombinant polypeptides comprising amino acid substitutions having an engineered C-terminal alpha-helical segment that stabilize the RSV F protein in a prefusion conformation.

The native sequence of RSV/B F protein (GenBank: WDV37446.1) is shown below with the (predicted) transmembrane region with italic and the C-terminal helix of the native sequence (residues 492-501) is also bold/underlined. The signal peptide is underlined with italic/underlined.

| (SEQ ID NO: 1242) | |
|---|---|
| 1 | MELLIHRSSA IFLTLAINAL YLTSSQNITE EFYQSTCSAV SRGYLSALRT |
| 51 | GWYTSVITIE LSNIKETKCN GTDTKVKLIK QELDKYKNAV TELQLLMQNT |
| 101 | PAVNNRARRE APQYMNYTIN TTKNLNVSIS KKRKRRFLGF LLGVGSAIAS |
| 151 | GIAVSKVLHL EGEVNKIKNA LQLTNKAVVS LSNGVSVLTS RVLDLKNYIN |
| 201 | NQLLPMVNRQ SCRISNIETV IEFQQKNSRL LEITREFSVN AGVTTPLSTY |
| 251 | MLTNSELLSL INDMPITNDQ KKLMSSNVQI VRQQSYSIMS IIKEEVLAYV |
| 301 | VQLPIYGVID TPCWKLHTSP LCTTNIKEGS NICLTRTDRG WYCDNAGSVS |
| 351 | FFPQADTCKV QSNRVFCDTM NSLTLPSEVS LCNTDIFNSK YDCKIMTSKT |
| 401 | DISSSVITSL GAIVSCYGKT KCTASNKNRG IIKTFSNGCD YVSNKGVDTV |
| 451 | SVGNTLYYVN KLEGKNLYVK GEPIINYYDP LVFPSDEFDA SISQVNEKIN |
| 501 | QSLAFIRRSD ELLHNVNTGK STTNIMITAI TIVIIVVLLS LIAIGLLLYC |
| 551 | KAKNTPVTLS KDQLSGINNI AFSK |

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 6, below, optionally lacking a p27 peptide shown in bold, and in which “X” refers to sites involving an added C-terminal helical segment and can be any amino acid:

(SEQ ID NO: 6)
QNITEEFYQSTCSAVSRGYLSALRTGWYTSVITIELSNIKETKCNGTDT
KVKLIKQELDKYKNAVTELQLLMQNTPAVNNRARREAPQYMNYTINTTK
NLNVSISKKRKRRFLGFLLGVGSAIASGIAVCKVLHLEGEVNKIKNALQ
LTNKAVVSLSNGVSVLTFRVLDLKNYINNQLLPMLNRQSCRISNIETVI
EFQQKNSRLLEITREFSVNAGVTTPLSTYMLINSELLSLINDMPITNDQ
KKLMSSNVQIVRQQSYSIMCIIKEEVLAYVVQLPIYGVIDTPCWKLHTS
PLCTTNIKEGSNICLTRTDRGWYCDNAGSVSFFPQADTCKVQSNRVFCD
TMNSLTLPSEVSLCNTDIFNSKYDCKIMTSKTDISSSVITSLGAIVSCY
GKTKCTASNKNRGIIKTFSNGCDYVSNKGVDTVSVGNTLYYVNKLEGKN
LYVKGEPIINYYDPLVFPSDEFDASISQVNEKINQSXXXXXXXXXXXXX
XXXX.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 7, below, optionally lacking a p27 peptide shown in bold, in which “X” refers to sites involving an added C-terminal helical segment and can be any amino acid:

(SEQ ID NO: 7)
QNITEEFYQSTCSAVSKGYLSALRTGWYTSVITIELSNIKENKCNGTDA
KVKLIKQELDKYKNAVTELQLLMQSTPATNNRARRELPRFMNYTLNNAK
KTNVTLSKKRKRRFLGFLLGVGSAIASGVAVCKVLHLEGEVNKIKSALL
STNKAVVSLSNGVSVLTFKVLDLKNYIDKQLLPILNKQSCSISNIETVI
EFQQKNNRLLEITREFSVNAGVTTPVSTYMLTNSELLSLINDMPITNDQ
KKLMSNNVQIVRQQSYSIMCIIKEEVLAYVVQLPLYGVIDTPCWKLHTS
PLCTTNTKEGSNICLTRTDRGWYCDNAGSVSFFPQAETCKVQSNRVFCD
TMNSLTLPSEVNLCNVDIFNPKYDCKIMTSKTDVSSSVITSLGAIVSCY
GKTKCTASNKNRGIIKTFSNGCDYVSNKGVDTVSVGNTLYYVNKQEGKS
LYVKGEPIINFYDPLVFPSDEFDASISQVNEKINQSXXXXXXXXXXXXX
XXXX.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 8, below, optionally lacking a p27 peptide shown in bold:

(SEQ ID NO: 8)
QNITEEFYQSTCSAVSRGYLSALRTGWYTSVITIELSNIKETKCNGTDT
KVKLIKQELDKYKNAVTELQLLMQNTPAVNNRARREAPQYMNYTINTTK
NLNVSISKKRKRRFLGFLLGVGSAIASGIAVCKVLHLEGEVNKIKNALQ
LTNKAVVSLSNGVSVLTFRVLDLKNYINNQLLPMLNRQSCRISNIETVI
EFQQKNSRLLEITREFSVNAGVTTPLSTYMLTNSELLSLINDMPITNDQ
KKLMSSNVQIVRQQSYSIMCIIKEEVLAYVVQLPIYGVIDTPCWKLHTS
PLCTTNIKEGSNICLTRTDRGWYCDNAGSVSFFPQADTCKVQSNRVFCD
TMNSLTLPSEVSLCNTDIFNSKYDCKIMTSKTDISSSVITSLGAIVSCY
GKTKCTASNKNRGIIKTFSNGCDYVSNKGVDTVSVGNTLYYVNKLEGKN
LYVKGEPIINYYDPLVFPSDEFDASISQVNEKINQSREIIRAINIVRKI
ASEK.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 9, below, optionally lacking a p27 peptide shown in bold:

(SEQ ID NO: 9)
QNITEEFYQSTCSAVSKGYLSALRTGWYTSVITIELSNIKENKCNGTDA
KVKLIKQELDKYKNAVTELQLLMQSTPATNNRARRELPRFMNYTLNNAK
KTNVTLSKKRKRRFLGFLLGVGSAIASGVAVCKVLHLEGEVNKIKSALL
STNKAVVSLSNGVSVLTFKVLDLKNYIDKQLLPILNKQSCSISNIETVI
EFQQKNNRLLEITREFSVNAGVTTPVSTYMLINSELLSLINDMPITNDQ
KKLMSNNVQIVRQQSYSIMCIIKEEVLAYVVQLPLYGVIDTPCWKLHTS
PLCTINTKEGSNICLTRTDRGWYCDNAGSVSFFPQAETCKVQSNRVFCD
TMNSLTLPSEVNLCNVDIFNPKYDCKIMTSKTDVSSSVITSLGAIVSCY
GKTKCTASNKNRGIIKTFSNGCDYVSNKGVDTVSVGNTLYYVNKQEGKS
LYVKGEPIINFYDPLVFPSDEFDASISQVNEKINQSREIIRAINIVRKI
ASEK.

In some embodiments, the polypeptide comprises a sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 1-9.

Illustrative sequences comprising various RSV F protein ectodomains and a C-terminal alpha-helical segment are shown in Table 4F. The signal peptide is underlined. The approximate region surrounding the p27 peptide is bold

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to sequences shown in Table 4F.

TABLE 4F

| | | SEQ ID |
|---|---|---|
| Sequence | Mutations | NO: |
| MELLILKANAITTILTAVTFCFASGQNITEEFYQSTCSAV | Introduced | 610 |
| SKGYLSALRTGWYTSVITIELSNIKENKCNGTDAKVKLI | mutations: | |
| KQELDKYKNAVTELQLLMQSTPACNNRARRELPRFM | T103C, I148C, | |
| NYTLNNAKKTNVTLSKKRKRRFLGFLLGVGSACASG | S190I, D486S | |
| VAVSKVLHLEGEVNKIKSALLSTNKAVVSLSNGVSVLT | Naturally occurring | |
| IKVLDLKNYIDKQLLPIVNKQSCSISNIETVIEFQQKNNR | substitutions: | |
| LLEITREFSVNAGVTTPVSTYMLTNSELLSLINDMPITN | P102A, I379V, | |
| DQKKLMSNNVQIVRQQSYSIMSIIKEEVLAYVVQLPLY | M447V | |
| GVIDTPCWKLHTSPLCTTNTKEGSNICLTRTDRGWYCD | | |
| NAGSVSFFPQAETCKVQSNRVFCDTMNSLTLPSEVNLC | | |
| NVDIFNPKYDCKIMTSKTDVSSSVITSLGAIVSCYGKTK | | |
| CTASNKNRGIIKTFSNGCDYVSNKGVDTVSVGNTLYY | | |
| VNKQEGKSLYVKGEPIINFYDPLVFPSSEFDASISQVNE | | |
| KINQSREIIRAINIVRKIASEK | | |
| MELLILKANAITTILTAVTFCFASGQNITEEFYQSTCSAV | Introduced | 611 |
| SKGYLSALRTGWYHSVITIELSNIKENKCNGTDAKVKL | mutations: | |
| IKQELDKYKNAVTELQLLMQSTPACNNRARRELPRFM | T54H, | |
| NYTLNNAKKTNVTLSKKRKRRFLGFLLGVGSACASG | T103C, I148C, | |
| VAVSKVLHLEGEVNKIKSALLSTNKAWSLSNGVSVLTI | S190I, V296I, | |
| KVLDLKNYIDKQLLPIVNKQSCSISNIETVIEFQQKNNR | D486S | |
| LLEITREFSVNAGVTTPVSTYMLTNSELLSLINDMPITN | Naturally occuring | |
| DQKKLMSNNVQIVRQQSYSIMSIIKEEILAYVVQLPLY | substitutions: | |
| GVIDTPCWKLHTSPLCTTNTKEGSNICLTRTDRGWYCD | P102A, I379V, | |
| NAGSVSFFPQAETCKVQSNRVFCDTMNSLTLPSEVNLC | M447V | |
| NVDIFNPKYDCKIMTSKTDVSSSVITSLGAIVSCYGKTK | | |
| CTASNKNRGIIKTFSNGCDYVSNKGVDTVSVGNTLYY | | |
| VNKQEGKSLYVKGEPIINFYDPLVFPSSEFDASISQVNE | | |
| KINQSREIIRAINIVRKIASEK | | |
| MELLILKANAITTILTAVTFCFASGQNITEEFYQSTCSAV | Introduced | 612 |
| SKGYLSALRTGWYHCVITIELSNIKENKCNGTDAKVKL | mutations: | |
| IKQELDKYKNAVTELQLLMQSTPATNNRARRELPRFM | T54H, S55C, | |
| NYTLNNAKKTNVTLSKKRKRRFLGFLLGVGSAIASG | L188C, D486S | |
| VAVSKVLHLEGEVNKIKSALLSTNKAVVSLSNGVSVC | Naturally occuring | |
| TSKVLDLKNYIDKQLLPIVNKQSCSISNIETVIEFQQKN | substitutions: | |
| NRLLEITREFSVNAGVTTPVSTYMLTNSELLSLINDMPI | P102A, I379V, | |
| TNDQKKLMSNNVQIVRQQSYSIMSIIKEEVLAYVVQLP | M447V | |
| LYGVIDTPCWKLHTSPLCTTNTKEGSNICLTRTDRGWY | | |
| CDNAGSVSFFPQAETCKVQSNRVFCDTMNSLTLPSEVN | | |
| LCNVDIFNPKYDCKIMTSKTDVSSSVITSLGAIVSCYGK | | |
| TKCTASNKNRGIIKTFSNGCDYVSNKGVDTVSVGNTL | | |
| YYVNKQEGKSLYVKGEPIINFYDPLVFPSSEFDASISQV | | |
| NEKINQSREIIRAINIVRKIASEK | | |
| MELLILKANAITTILTAVTFCFASGQNITEEFYQSTCSAV | Introduced | 613 |
| SKGYLSALRTGWYHCVITIELSNIKENKCNGTDAKVKL | mutations: | |
| IKQELDKYKNAVTELQLLMQSTPATNNRARRELPRFM | T54H, S55C, | |
| NYTLNNAKKTNVTLSKKRKRRFLGFLCGVGSAIASG | L142C, L188C, | |
| VAVSKVLHLEGEVNKIKSALLSTNKAVVSLSNGVSVC | V296I, N371C | |
| TSKVLDLKNYIDKQLLPIVNKQSCSISNIETVIEFQQKN | Naturally occuring | |
| NRLLEITREFSVNAGVTTPVSTYMLTNSELLSLINDMPI | substitutions: | |
| TNDQKKLMSNNVQIVRQQSYSIMSIIKEEILAYVVQLPL | P102A, I379V, | |
| YGVIDTPCWKLHTSPLCTTNTKEGSNICLTRTDRGWYC | M447V | |
| DNAGSVSFFPQAETCKVQSNRVFCDTMCSLTLPSEVNL | | |
| CNVDIFNPKYDCKIMTSKTDVSSSVITSLGAIVSCYGKT | | |
| KCTASNKNRGIIKTFSNGCDYVSNKGVDTVSVGNTLY | | |
| YVNKQEGKSLYVKGEPIINFYDPLVFPSDEFDASISQVN | | |
| EKINQSREIIRAINIVRKIASEK | | |
| MELLILKANAITTILTAVTFCFASGQNITEEFYQSTCSAV | Introduced | 614 |
| SKGYLSALRTGWYTCVITIELSNIKENKCNGTDAKVKL | mutations: | |
| IKQELDKYKNAVTELQLLMQSTPATNNRARRELPRFM | S55C, L188C, | |
| NYTLNNAKKTNVTLSKKRKRRFLGFLLGVGSAIASG | D486S | |
| VAVSKVLHLEGEVNKIKSALLSTNKAVVSLSNGVSVC | Naturally occuring | |
| TSKVLDLKNYIDKQLLPIVNKQSCSISNIETVIEFQQKN | substitutions: | |
| NRLLEITREFSVNAGVTTPVSTYMLTNSELLSLINDMPI | P102A, I379V, | |
| TNDQKKLMSNNVQIVRQQSYSIMSIIKEEVLAYWQLPL | M447V | |
| YGVIDTPCWKLHTSPLCTTNTKEGSNICLTRTDRGWYC | | |
| DNAGSVSFFPQAETCKVQSNRVFCDTMNSLTLPSEVNL | | |
| CNVDIFNPKYDCKIMTSKTDVSSSVITSLGAIVSCYGKT | | |
| KCTASNKNRGIIKTFSNGCDYVSNKGVDTVSVGNTLY | | |
| YVNKQEGKSLYVKGEPIINFYDPLVFPSSEFDASISQVN | | |
| EKINQSREIIRAINIVRKIASEK | | |
| MELLILKANAITTILTAVTFCFASGQNITEEFYQSTCSAV | Introduced | 615 |
| SKGYLSALRTGWYHCVITIELSNIKENKCNGTDAKVKL | mutations: | |
| IKQELDKYKNAVTELQLLMQSTPATNNRARRELPRFM | T54H, S55C, | |
| NYTLNNAKKTNVTLSKKRKRRFLGFLLGVGSAIASG | L188C, S190I | |
| VAVSKVLHLEGEVNKIKSALLSTNKAVVSLSNGVSVC | Naturally occuring | |
| TIKVLDLKNYIDKQLLPIVNKQSCSISNIETVIEFQQKNN | substitutions: | |
| RLLEITREFSVNAGVTTPVSTYMLTNSELLSLINDMPIT | P102A, I379V, | |
| NDQKKLMSNNVQIVRQQSYSIMSIIKEEVLAYVVQLPL | M447V | |
| YGVIDTPCWKLHTSPLCTTNTKEGSNICLTRTDRGWYC | | |
| DNAGSVSFFPQAETCKVQSNRVFCDTMNSLTLPSEVNL | | |
| CNVDIFNPKYDCKIMTSKTDVSSSVITSLGAIVSCYGKT | | |
| KCTASNKNRGIIKTFSNGCDYVSNKGVDTVSVGNTLY | | |
| YVNKQEGKSLYVKGEPIINFYDPLVFPSDEFDASISQVN | | |
| EKINQSREIIRAINIVRKIASEK | | |
| MELLILKANAITTILTAVTFCFASGQNITEEFYQSTCSAV | Introduced | 616 |
| SKGYLSALRTGWYTCVITIELSNIKENKCNGTDAKVKL | mutations: | |
| IKQELDKYKNAVTELQLLMQSTPATNNRARRELPRFM | S55C, L188C, | |
| NYTLNNAKKTNVTLSKKRKRRFLGFLLGVGSAIASG | S190I, D486S | |
| VAVSKVLHLEGEVNKIKSALLSTNKAVVSLSNGVSVC | Naturally occuring | |
| TIKVLDLKNYIDKQLLPIVNKQSCSISNIETVIEFQQKNN | substitutions: | |
| RLLEITREFSVNAGVTTPVSTYMLTNSELLSLINDMPIT | P102A, I379V, | |
| NDQKKLMSNNVQIVRQQSYSIMSIIKEEVLAYVVQLPL | M447V | |
| YGVIDTPCWKLHTSPLCTTNTKEGSNICLTRTDRGWYC | | |
| DNAGSVSFFPQAETCKVQSNRVFCDTMNSLTLPSEVNL | | |
| CNVDIFNPKYDCKIMTSKTDVSSSVITSLGAIVSCYGKT | | |
| KCTASNKNRGIIKTFSNGCDYVSNKGVDTVSVGNTLY | | |
| YVNKQEGKSLYVKGEPIINFYDPLVFPSSEFDASISQVN | | |
| EKINQSREIIRAINIVRKIASEK | | |
| MELLILKANAITTILTAVTFCFASGQNITEEFYQSTCSAV | Introduced | 617 |
| SKGYLSALRTGWYHCVITIELSNIKENKCNGTDAKVKL | mutations: | |
| IKQELDKYKNAVTELQLLMQSTPATNNRARRELPRFM | T54H, S55C, | |
| NYTLNNAKKTNVTLSKKRKRRFLGFLLGVGSAIASG | L188C, S190I, | |
| VAVSKVLHLEGEVNKIKSALLSTNKAVVSLSNGVSVC | D486S | |
| TIKVLDLKNYIDKQLLPIVNKQSCSISNIETVIEFQQKNN | Naturally occuring | |
| RLLEITREFSVNAGVTTPVSTYMLTNSELLSLINDMPIT | substitutions: | |
| NDQKKLMSNNVQIVRQQSYSIMSIIKEEVLAYVVQLPL | P102A, I379V, | |
| YGVIDTPCWKLHTSPLCTTNTKEGSNICLTRTDRGWYC | M447V | |
| DNAGSVSFFPQAETCKVQSNRVFCDTMNSLTLPSEVNL | | |
| CNVDIFNPKYDCKIMTSKTDVSSSVITSLGAIVSCYGKT | | |
| KCTASNKNRGIIKTFSNGCDYVSNKGVDTVSVGNTLY | | |
| YVNKQEGKSLYVKGEPIINFYDPLVFPSSEFDASISQVN | | |
| EKINQSREIIRAINIVRKIASEK | | |
| MELLILKANAITTILTAVTFCFASGQNITEEFYQSTCSAV | Introduced | 618 |
| SKGYLSALRTGWYTSVITIELSNIKENKCNGTDAKVKLI | mutations: | |
| KQELDKYKNAVTELQLLMQSTPATNNRARRELPRFM | S155C, S190I, | |
| NYTLNNAKKTNVTLSKKRKRRFLGFLLGVGSAIASG | S290C, D486S | |
| VAVCKVLHLEGEVNKIKSALLSTNKAVVSLSNGVSVL | Naturally occuring | |
| TIKVLDLKNYIDKQLLPIVNKQSCSISNIETVIEFQQKNN | substitutions: | |
| RLLEITREFSVNAGVTTPVSTYMLTNSELLSLINDMPIT | P102A, I379V, | |
| NDQKKLMSNNVQIVRQQSYSIMCIIKEEVLAYVVQLPL | M447V | |
| YGVIDTPCWKLHTSPLCTTNTKEGSNICLTRTDRGWYC | | |
| DNAGSVSFFPQAETCKVQSNRVFCDTMNSLTLPSEVNL | | |
| CNVDIFNPKYDCKIMTSKTDVSSSVITSLGAIVSCYGKT | | |
| KCTASNKNRGIIKTFSNGCDYVSNKGVDTVSVGNTLY | | |
| YVNKQEGKSLYVKGEPIINFYDPLVFPSSEFDASISQVN | | |
| EKINQSREIIRAINIVRKIASEK | | |
| MELLILKANAITTILTAVTFCFASGQNITEEFYQSTCSAV | Introduced | 619 |
| SKGYLSALRTGWYHCVITIELSNIKENKCNGTDAKVKL | mutations: | |
| IKQELDKYKNAVTELQLLMQSTPATNNRARRELPRFM | T54H, S55C, | |
| NYTLNNAKKTNVTLSKKRKRRFLGFLCGVGSAIASG | L142C, L188C, | |
| VAVSKVLHLEGEVNKIKSALLSTNKAVVSLSNGVSVC | V296I, N371C, | |
| TSKVLDLKNYIDKQLLPIVNKQSCSISNIETVIEFQQKN | D486S, E487Q, | |
| NRLLEITREFSVNAGVTTPVSTYMLTNSELLSLINDMPI | D489S | |
| TNDQKKLMSNNVQIVRQQSYSIMSIIKEEILAYVVQLPL | Naturally occuring | |
| YGVIDTPCWKLHTSPLCTTNTKEGSNICLTRTDRGWYC | substitutions: | |
| DNAGSVSFFPQAETCKVQSNRVFCDTMCSLTLPSEVNL | P102A, I379V, | |
| CNVDIFNPKYDCKIMTSKTDVSSSVITSLGAIVSCYGKT | M447V | |
| KCTASNKNRGIIKTFSNGCDYVSNKGVDTVSVGNTLY | | |
| YVNKQEGKSLYVKGEPIINFYDPLVFPSSQFSASISQVN | | |
| EKINQSREIIRAINIVRKIASEK | | |
| MELLILKANAITTILTAVTFCFASGQNITEEFYQSTCSAV | Introduced | 620 |
| SKGYLSALRTGWYHSVITIELSNIKENKCNGTDAKVKL | mutations: | |
| IKQELDKYKNAVTELQLLMQSTPATNNRARRELPRFM | T54H, S155C, | |
| NYTLNNAKKTNVTLSKKRKRRFLGFLLGVGSAIASG | S190I, S290C, | |
| VAVCKVLHLEGEVNKIKSALLSTNKAVVSLSNGVSVL | V296I | |
| TIKVLDLKNYIDKQLLPIVNKQSCSISNIETVIEFQQKNN | Naturally occuring | |
| RLLEITREFSVNAGVTTPVSTYMLTNSELLSLINDMPIT | substitutions: | |
| NDQKKLMSNNVQIVRQQSYSIMCIIKEEILAYWQLPLY | P102A, I379V, | |
| GVIDTPCWKLHTSPLCTTNTKEGSNICLTRTDRGWYCD | M447V | |
| NAGSVSFFPQAETCKVQSNRVFCDTMNSLTLPSEVNLC | | |
| NVDIFNPKYDCKIMTSKTDVSSSVITSLGAIVSCYGKTK | | |
| CTASNKNRGIIKTFSNGCDYVSNKGVDTVSVGNTLYY | | |
| VNKQEGKSLYVKGEPIINFYDPLVFPSDEFDASISQVNE | | |
| KINQSREIIRAINIVRKIASEK | | |
| MELLILKANAITTILTAVTFCFASGQNITEEFYQSTCSAV | | 621 |
| SKGYLSALRTGWYHCVITIELSNIKENKCNGTDAKVKL | | |
| IKQELDKYKNAVTELQLLMQSTPATNNRARRELPRFM | | |
| NYTLNNAKKTNVTLSKKRKRRFLGFLLGVGSAIASG | | |
| VAVSKVLHLEGEVNKIKSALLSTNKAVVSLSNGVSVC | | |
| TSKVLDLKNYIDKQLLPIVNKQSCSISNIETVIEFQQKN | | |
| NRLLEITREFSVNAGVTTPVSTYMLTNSELLSLINDMPI | | |
| TNDQKKLMSNNVQIVRQQSYSIMSIIKEEVLAYWQLPL | | |
| YGVIDTPCWKLHTSPLCTTNTKEGSNICLTRTDRGWYC | | |
| DNAGSVSFFPQAETCKVQSNRVFCDTMNSLTLPSEVNL | | |
| CNVDIFNPKYDCKIMTSKTDVSSSVITSLGAIVSCYGKT | | |
| KCTASNKNRGIIKTFSNGCDYVSNKGVDTVSVGNTLY | | |
| YVNKQEGKSLYVKGEPIINFYDPLVFPSSEFDASISQVN | | |
| EKINQSREIIRAINIVRKIASEK | | |
| MELLILKAAITTILTAVTFCFASGQNITEEFYQSTCSAVS | V56C + V164C | 622 |
| KGYLSALRTGWYTSCITIELSNIKENKCNGTDAVKLIK | | |
| QELDKYKNAVTELQLLMQSTPATNNRARRELPRFMY | | |
| TLNNAKKTVTLSKKRKRRFLGFLLGVGSAIASGVAV | | |
| SKVLHLEGECNKIKSALLSTNKAVVSLSNGVSVLTSKV | | |
| LDLKNYIDKQLLPIVKQSCSISNIETVIEFQQKNNRLLEI | | |
| TREFSVAGVTTPVSTYMLTNSELLSLINDMPITNDQKK | | |
| LMSNNVQIVRQQSYSIMSIIKEEVLAYWQLPLYGVIDTP | | |
| CWKLHTSPLCTTNTKEGSNICLTRTDRGWYCDNAGSV | | |
| SFFPQAETCKVQSNRVFCDTMSLTLPSEVNLCNVDIFN | | |
| PKYDCKIMTSKTDVSSSVITSLGAIVSCYGKTKCTASN | | |
| KNRGIIKTFSNGCDYVSNKGVDTVSVGNTLYYVKQEG | | |
| KSLYVKGEPIINFYDPLVFPSDEFDASISQVEKINQSREII | | |
| RAINIVRKIASEK | | |
| MELLILKAAITTILTAVTFCFASGQNITEEFYQSTCSAVS | I57C + S190C | 623 |
| KGYLSALRTGWYTSVCTIELSNIKENKCNGTDAVKLIK | | |
| QELDKYKNAVTELQLLMQSTPATNNRARRELPRFMY | | |
| TLNNAKKTVTLSKKRKRRFLGFLLGVGSAIASGVAV | | |
| SBVLHLEGEVKIKSALLSTNKAWSLSNGVSVLTCBVLD | | |
| LKNYIDKQLLPIVKQSCSISNIETVIEFQQKNNRLLEITR | | |
| EFSVAGVTTPVSTYMLTNSELLSLINDMPITNDQKKLM | | |
| SNNVQIVRQQSYSIMSIIKEEVLAYWQLPLYGVIDTPC | | |
| WKLHTSPLCTTNTKEGSNICLTRTDRGWYCDNAGSVS | | |
| FFPQAETCKVQSNRVFCDTMSLTLPSEVNLCNVDIFNP | | |
| KYDCKIMTSKTDVSSSVITSLGAIVSCYGKTKCTASNK | | |
| NRGIIKTFSNGCDYVSNKGVDTVSVGNTLYYVKQEGK | | |
| SLYVKGEPIINFYDPLVFPSDEFDASISQVEKINQSREIIR | | |
| AINIVRKIASEK | | |
| MELLILKAAITTILTAVTFCFASGQNITEEFYQSTCSAVS | T58C + V164C | 624 |
| KGYLSALRTGWYTSVICIELSNIKENKCNGTDAVKLIK | | |
| QELDKYKNAVTELQLLMQSTPATNNRARRELPRFMY | | |
| TLNNAKKTVTLSKKRKRRFLGFLLGVGSAIASGVAV | | |
| SKVLHLEGECNKIKSALLSTNKAVVSLSNGVSVLTSKV | | |
| LDLKNYIDKQLLPIVKQSCSISNIETVIEFQQKNNRLLEI | | |
| TREFSVAGVTTPVSTYMLTNSELLSLINDMPITNDQKK | | |
| LMSNNVQIVRQQSYSIMSIIKEEVLAYWQLPLYGVIDTP | | |
| CWKLHTSPLCTTNTKEGSNICLTRTDRGWYCDNAGSV | | |
| SFFPQAETCKVQSNRVFCDTMSLTLPSEVNLCNVDIFN | | |
| PKYDCKIMTSKTDVSSSVITSLGAIVSCYGKTKCTASN | | |
| KNRGIIKTFSNGCDYVSNKGVDTVSVGNTLYYVKQEG | | |
| KSLYVKGEPIINFYDPLVFPSDEFDASISQVEKINQSREII | | |
| RAINIVRKIASEK | | |
| MELLILKAAITTILTAVTFCFASGQNITEEFYQSTCSAVS | N165C + V296C | 625 |
| KGYLSALRTGWYTSVITIELSNIKENKCNGTDAVKLIK | | |
| QELDKYKNAVTELQLLMQSTPATNNRARRELPRFMY | | |
| TLNNAKKTVTLSKKRKRRFLGFLLGVGSAIASGVAV | | |
| SBVLHLEGEVCKIKSALLSTNKAWSLSNGVSVLTSBVL | | |
| DLKNYIDKQLLPIVKQSCSISNIETVIEFQQKNNRLLEIT | | |
| REFSVAGVTTPVSTYMLTNSELLSLINDMPITNDQKKL | | |
| MSNNVQIVRQQSYSIMSIIKEECLAYWQLPLYGVIDTPC | | |
| WKLHTSPLCTTNTKEGSNICLTRTDRGWYCDNAGSVS | | |
| FFPQAETCKVQSNRVFCDTMSLTLPSEVNLCNVDIFNP | | |
| KYDCKIMTSKTDVSSSVITSLGAIVSCYGKTKCTASNK | | |
| NRGIIKTFSNGCDYVSNKGVDTVSVGNTLYYVKQEGK | | |
| SLYVKGEPIINFYDPLVFPSDEFDASISQVEKINQSREIIR | | |
| AINIVRKIASEK | | |
| MELLILKAAITTILTAVTFCFASGQNITEEFYQSTCSAVS | K168C + V296C | 626 |
| KGYLSALRTGWYTSVITIELSNIKENKCNGTDAKVKLI | | |
| KQELDKYKNAVTELQLLMQSTPATIWRARRELPRFM | | |
| YTLAKKTVTLSKKRKRRFLGFLLGVGSAIASGVAVSB | | |
| VLHLEGEVKICSALLSTNKAWSLSNGVSVLTSBVLDLK | | |
| NYIDKQLLPIVKQSCSISNIETVIEFQQKNNRLLEITREFS | | |
| VAGVTTPVSTYMLTNSELLSLINDMPITNDQKKLMSNN | | |
| VQIVRQQSYSIMSIIKEECLAYWQLPLYGVIDTPCWKL | | |
| HTSPLCTTNTKEGSNICLTRTDRGWYCDNAGSVSFFPQ | | |
| AETCKVQSNRVFCDTMSLTLPSEVNLCNVDIFNPKYDC | | |
| KIMTSKTDVSSSVITSLGAIVSCYGKTKCTASNKNRGII | | |
| KTFSNGCDYVSNKGVDTVSVGNTLYYVKQEGKSLYV | | |
| KGEPIINFYDPLVFPSDEFDASISQVEKINQSREIIRAINIV | | |
| RKIASEK | | |
| MELLILKAAITTILTAVTFCFASGQNITEEFYQSTCSAVS | M396C + F483C | 627 |
| KGYLSALRTGWYTSVITIELSNIKENKCNGTDAVKLIK | | |
| QELDKYKNAVTELQLLMQSTPATNNRARRELPRFMY | | |
| TLNNAKKTVTLSKKRKRRFLGFLLGVGSAIASGVAV | | |
| SKVLHLEGEVKIKSALLSTNKAVVSLSNGVSVLTSKVL | | |
| DLKNYIDKQLLPIVKQSCSISNIETVIEFQQKNNRLLEIT | | |
| REFSVAGVTTPVSTYMLTNSELLSLINDMPITNDQKKL | | |
| MSNNVQIVRQQSYSIMSIIKEEVLAYWQLPLYGVIDTP | | |
| CWKLHTSPLCTTNTKEGSNICLTRTDRGWYCDNAGSV | | |
| SFFPQAETCKVQSNRVFCDTMSLTLPSEVNLCNVDIFN | | |
| PKYDCKICTSKTDVSSSVITSLGAIVSCYGKTKCTASNK | | |
| NRGIIKTFSNGCDYVSNKGVDTVSVGNTLYYVKQEGK | | |
| SLYVKGEPIINFYDPLVCPSDEFDASISQVEKINQSREIIR | | |
| AINIVRKIASEK | | |
| MELLILKANAITTILTAVTFCFASGQNITEEFYQSTCSAV | | 628 |
| SKGYLSALRTGWYTSVITIELSNIKKNKCNGTDAKVKL | | |
| IKQELDKYKNAVTELQLLMQSTQATNNRARRELPRF | | |
| MNYTLNNAKKTNVTLSKKRKRRFLGFLLGVGSAIAS | | |
| GVAVSKVLHLEGEVNKIKSALLSTNKAVVSLSNGVSV | | |
| LTSKVLDLKNYIDKQLLPIVNKQSCSISNIETVIEFQQKN | | |
| NRLLEITREFSVNAGVTTPVSTYMLTNSELLSLINDMPI | | |
| TNDQKKLMSNNVQIVRQQSYSIMSIIKEEVLAYVVQLP | | |
| LYGVIDTPCWKLHTSPLCTTNTKEGSNICLTRTDRGWY | | |
| CDNAGSVSFFPQAETCKVQSNRVFCDTMNSLTLPSEVN | | |
| LCNVDIFNPKYDCKIMTSKTDVSSSVITSLGAIVSCYGK | | |
| TKCTASNKNRGIIKTFSNGCDYVSNKGVDTVSVGNTL | | |
| YYVNKQEGKSLYVKGEPIINFYDPLVFPSDEFDASISQV | | |
| NEKINQSREIIRAINIVRKIASEK | | |
| MELLILKANAITTILTAVTFCFASGQNITEEFYQSTCSAV | | 629 |
| SKGYLSALRTGWYTSVITIELSNIKENKCNGTDAKVKLI | | |
| KQELDKYKNAVTELQLLMQSTPATNNRARRELPRFM | | |
| NYTLNNAKKTNVTLSKKRKRRFLGFLLGVGSAIASG | | |
| VAVCKVLHLEGEVNKIKSALLSTNKAVVSLSNGVSVL | | |
| TFKVLDLKNYIDKQLLPILNKQSCSISNIETVIEFQQKNN | | |
| RLLEITREFSVNAGVTTPVSTYMLTNSELLSLINDMPIT | | |
| NDQKKLMSNNVQIVRQQSYSIMCIIKEEVLAYVVQLPL | | |
| YGVIDTPCWKLHTSPLCTTNTKEGSNICLTRTDRGWYC | | |
| DNAGSVSFFPQAETCKVQSNRVFCDTMNSLTLPSEVNL | | |
| CNVDIFNPKYDCKIMTSKTDVSSSVITSLGAIVSCYGKT | | |
| KCTASNKNRGIIKTFSNGCDYVSNKGVDTVSVGNTLY | | |
| YVNKQEGKSLYVKGEPIINFYDPLVFPSDEFDASISQVN | | |
| EKINQSREIIRAINIVRKIASEK | | |
| MELLILKANAITTILTAVTFCFASGQNITEEFYQSTCSAV | | 630 |
| SKGYLSALRTGWYTSVITIELSNIKKNKCNGTDAKVKL | | |
| IKQELDKYKNAVTELQLLMQSTQATNNRARRELPRF | | |
| MNYTLNNAKKTNVTLSKKRKRRFLGFLLGVGSAIAS | | |
| GVAVSKVLHLEGEVNKIKSALLSTNKAVVSLSNGVSV | | |
| LTSKVLDLKNYIDKQLLPIVNKQSCSISNIETVIEFQQKN | | |
| NRLLEITREFSVNAGVTTPVSTYMLTNSELLSLINDMPI | | |
| TNDQKKLMSNNVQIVRQQSYSIMSIIKEEVLAYVVQLP | | |
| LYGVIDTPCWKLHTSPLCTTNTKEGSNICLTRTDRGWY | | |
| CDNAGSVSFFPQAETCKVQSNRVFCDTMNSLTLPSEVN | | |
| LCNVDIFNPKYDCKIMTSKTDVSSSVITSLGAIVSCYGK | | |
| TKCTASNKNRGIIKTFSNGCDYVSNKGVDTVSVGNTL | | |
| YYVNKQEGKSLYVKGEPIINFYDPLVFPSDEFDASISQV | | |
| NEKINQSREIIRAINIVRKIASEK | | |
| MELLILKANAITTILTAVTFCFASGQNITEEFYQSTCSAV | DS-Cav1 | 631 |
| SKGYLSALRTGWYTSVITIELSNIKENKCNGTDAKVKLI | | |
| KQELDKYKNAVTELQLLMQSTPATNNRARRELPRFM | | |
| NYTLNNAKKTNVTLSKKRKRRFLGFLLGVGSAIASG | | |
| VAVCKVLHLEGEVNKIKSALLSTNKAVVSLSNGVSVL | | |
| TFKVLDLKNYIDKQLLPILNKQSCSISNIETVIEFQQKNN | | |
| RLLEITREFSVNAGVTTPVSTYMLTNSELLSLINDMPIT | | |
| NDQKKLMSNNVQIVRQQSYSIMCIIKEEVLAYVVQLPL | | |
| YGVIDTPCWKLHTSPLCTTNTKEGSNICLTRTDRGWYC | | |
| DNAGSVSFFPQAETCKVQSNRVFCDTMNSLTLPSEVNL | | |
| CNVDIFNPKYDCKIMTSKTDVSSSVITSLGAIVSCYGKT | | |
| KCTASNKNRGIIKTFSNGCDYVSNKGVDTVSVGNTLY | | |
| YVNKQEGKSLYVKGEPIINFYDPLVFPSDEFDASISQVN | | |
| EKINQSREIIRAINIVRKIASEK | | |
| MELLILKANAITTILTAVTFCFASGQNITEEFYQSTCSAV | | 632 |
| SKGYLSALRTGWYTSVITIELSNIKKNKCNGTDAKVKL | | |
| IKQELDKYKNAVTELQLLMQSTQATNNRARQQQQRFL | | |
| GFLLGVGSAIASGVAVSKVLHLEGEVNKIKSALLSTNK | | |
| AVVSLSNGVSVLTSKVLDLKNYIDKQLLPIVNKQSCSIS | | |
| NIETVIEFQQKNNRLLEITREFSVNAGVTTPVSTYMLTN | | |
| SELLSLINDMPITNDQKKLMSNNVQIVRQQSYSIMSIIK | | |
| EEVLAYVVQLPLYGVIDTPCWKLHTSPLCTTNTKEGSN | | |
| ICLTRTDRGWYCDNAGSVSFFPQAETCKVQSNRVFCD | | |
| TMNSLTLPSEVNLCNVDIFNPKYDCKIMTSKTDVSSSVI | | |
| TSLGAIVSCYGKTKCTASNKNRGIIKTFSNGCDYVSNK | | |
| GVDTVSVGNTLYYVNKQEGKSLYVKGEPIINFYDPLVF | | |
| PSDEFDASISQVNEKINQSREIIRAINIVRKIASEK | | |
| MELLILKTNAITAILAAVTLCFASSQNITEEFYQSTCSAV | Deletion of p27 | 633 |
| SKGYLSALRTGWYTSVITIELSNIKENKCNGTDAKVKLI | sequence | |
| KQELDKYKSAVTELQLLMQSTPATNNKFLGFLLGVGS | | |
| AIASGIAVSKVLHLEGEVNKIKSALLSTNKAVVSLSNG | | |
| VSVLTSKVLDLKNYIDKQLLPIVNKQSCSISNIETVIEFQ | | |
| QKNNRLLEITREFSVNAGVTTPVSTYMLTNSELLSLIND | | |
| MPITNDQKKLMSNNVQIVRQQSYSIMSIIKEEVLAYVV | | |
| QLPLYGVIDTPCWKLHTSPLCTTNTKEGSNICLTRTDR | | |
| GWYCDNAGSVSFFPLAETCKVQSNRVFCDTMNSLTLP | | |
| SEVNLCNIDIFNPKYDCKIMTSKTDVSSSVITSLGAIVSC | | |
| YGKTKCTASNKNRGIIKTFSNGCDYVSNKGVDTVSVG | | |
| NTLYYVNKQEGKSLYVKGEPIINFYDPLVFPSDEFDASI | | |
| SQVNEKINQSREIIRAINIVRKIASEK | | |
| MELLILKANAITTILTAVTFCFASGQNITEEFYQSTCSAV | P27 mutation | 634 |
| SKGYLSALRTGWYTSVITIELSNIKENKCNGTDAKVKLI | | |
| KQELDKYKNAVTELQLLMQSTPATNNRARRELPRFMN | | |
| YTLNNAKKTNVTLSKKQKQQAIASGVAVSKVLHLEGE | | |
| VNKIKSALLSTNKAVVSLSNGVSVLTSKVLDLKNYIDK | | |
| QLLPIVNKQSCSISNIETVIEFQQKNNRLLEITREFSVNA | | |
| GVTTPVSTYMLTNSELLSLINDMPITNDQKKLMSNNVQ | | |
| IVRQQSYSIMSIIKEEVLAYVVQLPLYGVIDTPCWKLHT | | |
| SPLCTTNTKEGSNICLTRTDRGWYCDNAGSVSFFPQAE | | |
| TCKVQSNRVFCDTMNSLTLPSEVNLCNVDIFNPKYDC | | |
| KIMTSKTDVSSSVITSLGAIVSCYGKTKCTASNKNRGII | | |
| KTFSNGCDYVSNKGVDTVSVGNTLYYVNKQEGKSLY | | |
| VKGEPIINFYDPLVFPSDEFDASISQVNEKINQSREIIRAI | | |
| NIVRKIASEK | | |
| METPAQLLFLLLLWLPDTTGFASGQNITEEFYQSTCSA | Deletion of p27 | 635 |
| VSKGYLSALRTGWYTSVITIELSNIKKNKCNGTDAKVK | sequence | |
| LIKQELDKYKNAVTELQLLMQSTQATNNRARQQQQRF | | |
| LGFLLGVGSAIASGVAVSKVLHLEGEVNKIKSALLSTN | | |
| KAVVSLSNGVSVLTSKVLDLKNYIDKQLLPIVNKQSCS | | |
| ISNIETVIEFQQKNNRLLEITREFSVNAGVTTPVSTYMLT | | |
| NSELLSLINDMPITNDQKKLMSNNVQIVRQQSYSIMSII | | |
| KEEVLAYVVQLPLYGVIDTPCWKLHTSPLCTTNTKEGS | | |
| NICLTRTDRGWYCDNAGSVSFFPQAETCKVQSNRVFC | | |
| DTMNSLTLPSEVNLCNVDIFNPKYDCKIMTSKTDVSSS | | |
| VITSLGAIVSCYGKTKCTASNKNRGIIKTFSNGCDYVSN | | |
| KGVDTVSVGNTLYYVNKQEGKSLYVKGEPIINFYDPL | | |
| VFPSDEFDASISQVNEKINQSREIIRAINIVRKIASEK | | |
| METPAQLLFLLLLWLPDTTGFASGQNITEEFYQSTCSA | Deletion of p27 | 636 |
| VSKGYLSALRTGWYTSVITIELSNIKKNKCNGTDAKVK | sequence | |
| LIKQELDKYKNAVTELQLLMQSTQATNNRARQQQQRF | | |
| LGFLLGVGSAIASGVAVSKVLHLEGEVNKIKSALLSTN | | |
| KAVVSLSNGVSVLTSKVLDLKNYIDKQLLPIVNKQSCS | | |
| ISNIETVIEFQQKNNRLLEITREFSVNAGVTTPVSTYMLT | | |
| NSELLSLINDMPITNDQKKLMSNNVQIVRQQSYSIMSII | | |
| KEEVLAYVVQLPLYGVIDTPCWKLHTSPLCTTNTKEGS | | |
| NICLTRTDRGWYCDNAGSVSFFPQAETCKVQSNRVFC | | |
| DTMNSLTLPSEVNLCNVDIFNPKYDCKIMTSKTDVSSS | | |
| VITSLGAIVSCYGKTKCTASNKNRGIIKTESNGCDYVSN | | |
| KGVDTVSVGNTLYYVNKQEGKSLYVKGEPIINFYDPL | | |
| VFPSDEFDASISQVNEKINQSREIIRAINIVRKIASEK | | |
| MELLILKANAITTILTAVTFCFASGQNITEEFYQSTCSAV | DS-Cav1 | 637 |
| SKGYLSALRTGWYTSVITIELSNIKENKCNGTDAKVKLI | | |
| KQELDKYKNAVTELQLLMQSTPATNNRARRELPRFM | | |
| NYTLNNAKKTNVTLSKKRKRRFLGFLLGVGSAIASG | | |
| VAVCKVLHLEGEVNKIKSALLSTNKAVVSLSNGVSVL | | |
| TFKVLDLKNYIDKQLLPILNKQSCSISNIETVIEFQQKNN | | |
| RLLEITREFSVNAGVTTPVSTYMLTNSELLSLINDMPIT | | |
| NDQKKLMSNNVQIVRQQSYSIMCIIKEEVLAYVVQLPL | | |
| YGVIDTPCWKLHTSPLCTTNTKEGSNICLTRTDRGWYC | | |
| DNAGSVSFFPQAETCKVQSNRVFCDTMNSLTLPSEVNL | | |
| CNVDIFNPKYDCKIMTSKTDVSSSVITSLGAIVSCYGKT | | |
| KCTASNKNRGIIKTFSNGCDYVSNKGVDTVSVGNTLY | | |
| YVNKQEGKSLYVKGEPIINFYDPLVFPSDEFDASISQVN | | |
| EKINQSREIIRAINIVRKIASEK | | |
| MELLILKANAITTILTAVTFCFASQNITEEFYQSTCSAVS | | 638 |
| KGYLSALRTGWYTSVITIELSNIKENKCNGTDAKVKLI | | |
| KQELDKYKNAVTELQLLMQSTPATNNRARRELPRFMN | | |
| YTLNNAKKINVILSKKRKRRFLGFLLGVGSAIASGVAV | | |
| CKVLHLEGEVNKIKSALLSINKAVVSLSNGVSVLIFKVL | | |
| DLKNYIDKQLLPILNKQSCSISNIETVIEFQQKNNRLLEI | | |
| TREFSVNAGVITPVSTYMLINSELLSLINDMPITNDQKK | | |
| LMSNNVQIVRQQSYSIMCIIKEEVLAYVVQLPLYGVID | | |
| TPCWKLHISPLCTINTKEGSNICLTRIDRGWYCDNAGS | | |
| VSFFPQAETCKVQSNRVFCDTMNSLTLPSEVNLCNVDI | | |
| FNPKYDCKIMISKTDVSSSVITSLGAIVSCYGKTKCIAS | | |
| NKNRGIIKTFSNGCDYVSNKGVDTVSVGNTLYYVNKQ | | |
| EGKSLYVKGEPIINFYDPLVFPSDEFDASISQVNEKINQS | | |
| REIIRAINIVRKIASEK | | |

In some embodiments, the ectodomain comprises any of the stabilizing mutations of RSV F protein disclosed in U.S. Pat. Nos. 9,950,058, 8,563,002, 11,261,239, 11,629,181, and 11,655,284, each of which is hereby incorporated by reference in its entirety.

### Furin Cleavage Site

RSV F proteins are cleaved during expression by the protease furin. Constructs that replace the cleavage site for furin with a glycine-serine linker are provided herein. Sequences are provided in Table 5A. In some embodiments, RSV F protein ectodomain comprises an uncleaved furin cleavage site.

TABLE 5A
Furin cleavage linkers

| Sequence | Length | SEQ ID NO: |
|---|---|---|
| NNQARGSGSGRSLGF | 15 | 639 |
| NNQARGGSGGRSLGF | 15 | 640 |
| NNGARGGSGGRSLGF | 15 | 641 |
| NNQARGGSGGDSLGF | 15 | 642 |
| NNQARGGSGSGGDSLGF | 17 | 643 |
| NNQARGGSGGGDLG | 14 | 644 |
| NNQARGGSGSGGDLGF | 16 | 645 |

### Linker

In some embodiments, the recombinant polypeptide and a protein nanostructure may be genetically fused such that they are both present in a single polypeptide, termed a “fusion protein.” The linkage between the polypeptide and the protein nanostructure allows the recombinant polypeptide to be displayed on the exterior of the self-assembling protein nanostructure.

A wide variety of polypeptide sequences can be used to link the proteins, or antigenic fragments thereof and the protein nanostructure. In some cases the linker comprises a polypeptide sequence that can be included in the encoding polynucleotide sequence. Any suitable linker polypeptide can be used. In some embodiments, the linker imposes a rigid relative orientation of the antigenic protein (e.g., ectodomain from the RSV Fusion protein) or antigenic fragment thereof to the protein nanostructure. In some embodiments, the linker flexibly links the antigenic protein (e.g., ectodomain from the RSV Fusion protein) or antigenic fragment thereof to the protein nanostructure. In some embodiments, the encoded polypeptides can include a linker between regions. In some embodiments, the polypeptide is a fusion protein which includes the recombinant RSV polypeptide, a linker, and the protein nanostructure component polypeptide. In some embodiments, the polypeptide is a fusion protein, which includes, in N- to C-terminal order, the recombinant RSV polypeptide, a linker, and the protein nanostructure component polypeptide. The linker can be a polypeptide. A wide variety of polypeptide sequences can be used and are well known in the art. In some embodiments, the linker may comprise a Gly-Ser linker (i.e., a linker consisting of glycine and serine residues) of any suitable length. In some embodiments, the Gly-Ser linker may be 3, 4, 5, 6, 7, 8, 9, 10, 11, 12, 13, 14, 15, 16, 17, 18, 19, 20 or more amino acid residues in length. Non-limiting examples of Glys-Ser linkers are presented in Table 5B.

TABLE 5B

| Sequence | Length | SEQ ID NO: |
|---|---|---|
| GSS | 3 | 646 |
| GSGS | 4 | 647 |
| GGSGEKP | 7 | 648 |
| GGSGQKP | 7 | 649 |
| GGSGGSGS | 8 | 650 |
| GGSGGSGEKP | 10 | 651 |
| GGSGGSGQKP | 10 | 652 |
| GGSGGSGGSGGS | 12 | 653 |
| GSGGSGSGSGGS | 12 | 654 |
| GGGGGSGGGSGGGGS | 15 | 655 |
| GGGGSGGGGSGGGGS | 15 | 656 |
| GGSGGSGSGGSGGSGS | 16 | 657 |
| GGGGSGGGGSGGGGSGG | 17 | 658 |
| SGGGSGGSGSGGSGGSGS | 18 | 659 |
| EPEGGSGGSGSGGSGGSGS | 19 | 660 |
| YGGSGGSGGSGSGGSGGSGS | 20 | 661 |
| GGSGGSGSGGSGGSGSGGSGSGGS | 24 | 662 |
| GSGGSGGSGGSGGSGSGGSGGSGS | 24 | 663 |
| KSDELLGSGGSGSGSGGSEKAAKAEEAARK | 30 | 664 |

In some embodiments, the linker comprises between 3 and 30 amino acid residues. In some embodiments, the linker comprises between 4 and 24 amino acid residues. In some embodiments, the linker comprises between 8 and 24 amino acid residues. In some embodiments, the linker comprises between 10 and 24 amino acid residues. In some embodiments, the linker comprises between 12 and 24 amino acid residues. In some embodiments, the linker comprises between 16 and 24 amino acid residues. In some embodiments, the linker comprises between 18 and 24 amino acid residues. In some embodiments, the linker comprises between 20 and 24 amino acid residues. In some embodiments, the linker comprises between 4 and 20 amino acid residues. In some embodiments, the linker comprises between 8 and 20 amino acid residues. In some embodiments, the linker comprises between 10 and 20 amino acid residues. In some embodiments, the linker comprises between 12 and 20 amino acid residues. In some embodiments, the linker comprises between 16 and 20 amino acid residues. In some embodiments, the linker comprises between 8 and 18 amino acid residues. In some embodiments, the linker comprises between 12 and 16 amino acid residues. In some embodiments, the linker comprises 3 amino acid residues. In some embodiments, the linker comprises 4 amino acid residues. In some embodiments, the linker comprises 5 amino acid residues. In some embodiments, the linker comprises 6 amino acid residues. In some embodiments, the linker comprises 7 amino acid residues. In some embodiments, the linker comprises 8 amino acid residues. In some embodiments, the linker comprises 8 amino acid residues. In some embodiments, the linker comprises 10 amino acid residues. In some embodiments, the linker comprises 11 amino acid residues. In some embodiments, the linker comprises 12 amino acid residues. In some embodiments, the linker comprises 13 amino acid residues. In some embodiments, the linker comprises 14 amino acid residues. In some embodiments, the linker comprises 15 amino acid residues. In some embodiments, the linker comprises 16 amino acid residues. In some embodiments, the linker comprises 17 amino acid residues. In some embodiments, the linker comprises 18 amino acid residues. In some embodiments, the linker comprises 19 amino acid residues. In some embodiments, the linker comprises 20 amino acid residues. In some embodiments, the linker comprises 21 amino acid residues. In some embodiments, the linker comprises 22 amino acid residues. In some embodiments, the linker comprises 23 amino acid residues. In some embodiments, the linker comprises 24 amino acid residues. In some embodiments, the linker comprises 25 amino acid residues. In some embodiments, the linker comprises 26 amino acid residues. In some embodiments, the linker comprises 27 amino acid residues. In some embodiments, the linker comprises 28 amino acid residues. In some embodiments, the linker comprises 29 amino acid residues. In some embodiments, the linker comprises 30 amino acid residues.

In some embodiments, the encoded polypeptides can include a linker between regions. In some embodiments, the polypeptide is a fusion protein which includes the recombinant RSV polypeptide, a linker, a N-terminal extension linker, and the protein nanostructure component polypeptide. In some embodiments, the polypeptide is a fusion protein, which includes, in N- to C-terminal order, the recombinant RSV polypeptide, a linker, a N-terminal extension linker, and the protein nanostructure component polypeptide. In some embodiments, the N-terminal extension linker is I53-50A helical extension. In some embodiments, polypeptide sequence of N-terminal extension linker is EKAAKAEEAARK (SEQ ID NO: 665).

### Trimerization Domains

In some embodiments, the polypeptide may comprise a trimerization domain, such as FoldOn or a GCN4 trimerization. In some embodiments, the linker sequence comprises a FoldOn, wherein the FoldOn sequence is GYIPEAPRDGQAYVRKDGEWVLLSTFL (SEQ ID NO: 1235).

In some embodiments, the polypeptide may comprise a trimerization domain, wherein the trimerization domain sequence is DKIEEILSKIYHIENEIARIKKLIGE (SEQ ID NO: 666) (GEN). In some embodiments, the polypeptide may comprise a trimerization domain, wherein the trimerization domain sequence is EKFHQIEKEFSEVEGRIQDLEK (SEQ ID NO: 667) (HA).

In some embodiments, the polypeptide may comprise a trimerization domain, wherein the trimerization domain sequence is EDKIEEILSKIYHIENEIARIKKLIGEA (Seq ID NO: 668) (coiled-coil isoleucine zipper).

In some embodiments, the polypeptide may comprise a trimerization domain, wherein the trimerization domain sequence is GSGYIPEAPRDGQAYVRKDGEWVLLSTFL (SEQ ID NO: 669) (bacteriophage T4 fibritin).

In some embodiments, a trimerization sequence is RMKQIEDKIEEILSKIYHIENEIARIKKLIGEA (SEQ ID NO: 670) (GCN4). In some embodiments, a trimerization domain is a GCN4 variant. In some embodiments, the GCN4 variant sequence is RMKQIEDKIEEILSKIYHIENEIARIKKLIGERGGR (SEQ ID NO: 671), RMKQIEDKIEEILSKIYHIENEIARIKKLIGNRTGGR (SEQ ID NO: 672), RMKQIEDKIENITSKIYHIENEIARIKKLIGNRTGGR (SEQ ID NO: 673), RMKQIEDKIEEILSKIYNITNEIARIKKLIGNRTGGR (SEQ ID NO: 674), or RMKQIEDKIENITSKIYNITNEIARIKKLIGNRTGGR (SEQ ID NO: 675).

Illustrative sequences comprising various RSV F protein ectodomains, a C-terminal alpha-helical segment, and FoldOn are shown in Table 5C. The signal peptide is underlined with italic. The underlined FoldOn sequence may be substituted with any one of the trimerization domains described herein or any one of the multimerization domains described in Table 11 to generate embodiments that comprise such other trimerization domains.

In some embodiments, the trimeric protein complex comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to sequences shown in Table 5C. In some embodiments, the trimeric protein complex can be used as a trimeric component of a protein nanostructure. The approximate region surrounding the p27 peptide is bold. In some embodiments, the p27 peptide may be removed from the RSV F protein ectodomain through furin-based cleavage during production of antigens in cell culture. The FoldOn sequence is bold/underlined.

TABLE 5C

| | | SEQ ID |
|---|---|---|
| Sequence | Mutations | NO: |
| MELLILKANAITTILTAVTFCFASGQNITEEFYQSTCSA | Introduced mutations: | 676 |
| VSKGYLSALRTGWYTSVITIELSNIKENKCNGTDAK | T103C, I148C, S190I, | |
| VKLIKQELDKYKNAVTELQLLMQSTPACNNRARRE | D486S | |
| LPRFMNYTLNNAKKTNVTLSKKRKRRFLGFLLG | Naturally occurring | |
| VGSACASGVAVSKVLHLEGEVNKIKSALLSTNKAV | substitutions: | |
| VSLSNGVSVLTIKVLDLKNYIDKQLLPIVNKQSCSIS | P102A, I379V, | |
| NIETVIEFQQKNNRLLEITREFSVNAGVTTPVSTYML | M447V | |
| TNSELLSLINDMPITNDQKKLMSNNVQIVRQQSYSI | | |
| MSIIKEEVLAYVVQLPLYGVIDTPCWKLHTSPLCTT | | |
| NTKEGSNICLTRTDRGWYCDNAGSVSFFPQAETCK | | |
| VQSNRVFCDTMNSLTLPSEVNLCNVDIFNPKYDCKI | | |
| MTSKTDVSSSVITSLGAIVSCYGKTKCTASNKNRGII | | |
| KTFSNGCDYVSNKGVDTVSVGNTLYYVNKQEGKS | | |
| LYVKGEPIINFYDPLVFPSSEFDASISQVNEKINQSRE | | |
| IIRAINIVRKIASEKSAIGGYIPEAPRDGQAYVRKD | | |
| GEWVLLSTFL | | |
| MELLILKANAITTILTAVTFCFASGQNITEEFYQSTCSA | Introduced mutations: | 677 |
| VSKGYLSALRTGWYHSVITIELSNIKENKCNGTDAK | T54H, T103C, I148C, | |
| VKLIKQELDKYKNAVTELQLLMQSTPACNNRARRE | S190I, V296I, D486S | |
| LPRFMNYTLNNAKKTNVTLSKKRKRRFLGFLLG | Naturally occurring | |
| VGSACASGVAVSKVLHLEGEVNKIKSALLSTNKAW | substitutions: | |
| SLSNGVSVLTIKVLDLKNYIDKQLLPIVNKQSCSISN | P102A, I379V, | |
| IETVIEFQQKNNRLLEITREFSVNAGVTTPVSTYMLT | M447V | |
| NSELLSLINDMPITNDQKKLMSNNVQIVRQQSYSIM | | |
| SIIKEEILAYVVQLPLYGVIDTPCWKLHTSPLCTTNT | | |
| KEGSNICLTRTDRGWYCDNAGSVSFFPQAETCKVQ | | |
| SNRVFCDTMNSLTLPSEVNLCNVDIFNPKYDCKIMT | | |
| SKTDVSSSVITSLGAIVSCYGKTKCTASNKNRGIIKT | | |
| FSNGCDYVSNKGVDTVSVGNTLYYVNKQEGKSLY | | |
| VKGEPIINFYDPLVFPSSEFDASISQVNEKINQSREIIR | | |
| AINIVRKIASEKSAIGGYIPEAPRDGQAYVRKDGE | | |
| WVLLSTFL | | |
| MELLILKANAITTILTAVTFCFASGQNITEEFYQSTCSA | Introduced mutations: | 678 |
| VSKGYLSALRTGWYHCVITIELSNIKENKCNGTDA | T54H, S55C, L188C, | |
| KVKLIKQELDKYKNAVTELQLLMQSTPATNNRARR | D486S | |
| ELPRFMNYTLNNAKKTNVTLSKKRKRRFLGFLL | Naturally occurring | |
| GVGSAIASGVAVSKVLHLEGEVNKIKSALLSTNKA | substitutions: | |
| VVSLSNGVSVCTSKVLDLKNYIDKQLLPIVNKQSCS | P102A, I379V, | |
| ISNIETVIEFQQKNNRLLEITREFSVNAGVTTPVSTY | M447V | |
| MLTNSELLSLINDMPITNDQKKLMSNNVQIVRQQS | | |
| YSIMSIIKEEVLAYVVQLPLYGVIDTPCWKLHTSPLC | | |
| TTNTKEGSNICLTRTDRGWYCDNAGSVSFFPQAET | | |
| CKVQSNRVFCDTMNSLTLPSEVNLCNVDIFNPKYD | | |
| CKIMTSKTDVSSSVITSLGAIVSCYGKTKCTASNKN | | |
| RGIIKTFSNGCDYVSNKGVDTVSVGNTLYYVNKQE | | |
| GKSLYVKGEPIINFYDPLVFPSSEFDASISQVNEKIN | | |
| QSREIIRAINIVRKIASEKSAIGGYIPEAPRDGQAYV | | |
| RKDGEWVLLSTFL | | |
| MELLILKANAITTILTAVTFCFASGQNITEEFYQSTCSA | Introduced mutations: | 679 |
| VSKGYLSALRTGWYHCVITIELSNIKENKCNGTDA | T54H, S55C, L142C, | |
| KVKLIKQELDKYKNAVTELQLLMQSTPATNNRARR | L188C, V296I, N371C | |
| ELPRFMNYTLNNAKKTNVTLSKKRKRRFLGFLC | Naturally occurring | |
| GVGSAIASGVAVSKVLHLEGEVNKIKSALLSTNKA | substitutions: | |
| VVSLSNGVSVCTSKVLDLKNYIDKQLLPIVNKQSCS | P102A, I379V, | |
| ISNIETVIEFQQKNNRLLEITREFSVNAGVTTPVSTY | M447V | |
| MLTNSELLSLINDMPITNDQKKLMSNNVQIVRQQS | | |
| YSIMSIIKEEILAYVVQLPLYGVIDTPCWKLHTSPLC | | |
| TTNTKEGSNICLTRTDRGWYCDNAGSVSFFPQAET | | |
| CKVQSNRVFCDTMCSLTLPSEVNLCNVDIFNPKYD | | |
| CKIMTSKTDVSSSVITSLGAIVSCYGKTKCTASNKN | | |
| RGIIKTFSNGCDYVSNKGVDTVSVGNTLYYVNKQE | | |
| GKSLYVKGEPIINFYDPLVFPSDEFDASISQVNEKIN | | |
| QSREIIRAINIVRKIASEKSAIGGYIPEAPRDGQAYV | | |
| RKDGEWVLLSTFL | | |
| MELLILKANAITTILTAVTFCFASGQNITEEFYQSTCSA | Introduced mutations: | 680 |
| VSKGYLSALRTGWYTCVITIELSNIKENKCNGTDAK | S55C, L188C, D486S | |
| VKLIKQELDKYKNAVTELQLLMQSTPATNNRARRE | Naturally occurring | |
| LPRFMNYTLNNAKKTNVTLSKKRKRRFLGFLLG | substitutions: | |
| VGSAIASGVAVSKVLHLEGEVNKIKSALLSTNKAV | P102A, I379V, | |
| VSLSNGVSVCTSKVLDLKNYIDKQLLPIVNKQSCSIS | M447V | |
| NIETVIEFQQKNNRLLEITREFSVNAGVTTPVSTYML | | |
| TNSELLSLINDMPITNDQKKLMSNNVQIVRQQSYSI | | |
| MSIIKEEVLAYWQLPLYGVIDTPCWKLHTSPLCTTN | | |
| TKEGSNICLTRTDRGWYCDNAGSVSFFPQAETCKV | | |
| QSNRVFCDTMNSLTLPSEVNLCNVDIFNPKYDCKI | | |
| MTSKTDVSSSVITSLGAIVSCYGKTKCTASNKNRGII | | |
| KTFSNGCDYVSNKGVDTVSVGNTLYYVNKQEGKS | | |
| LYVKGEPIINFYDPLVFPSSEFDASISQVNEKINQSRE | | |
| IIRAINIVRKIASEKSAIGGYIPEAPRDGQAYVRKD | | |
| GEWVLLSTFL | | |
| MELLILKANAITTILTAVTFCFASGQNITEEFYQSTCSA | Introduced mutations: | 681 |
| VSKGYLSALRTGWYHCVITIELSNIKENKCNGTDA | T54H, S55C, L188C, | |
| KVKLIKQELDKYKNAVTELQLLMQSTPATNNRARR | S190I | |
| ELPRFMNYTLNNAKKTNVTLSKKRKRRFLGFLL | Naturally occurring | |
| GVGSAIASGVAVSKVLHLEGEVNKIKSALLSTNKA | substitutions: | |
| VVSLSNGVSVCTIKVLDLKNYIDKQLLPIVNKQSCSI | P102A, I379V, | |
| SNIETVIEFQQKNNRLLEITREFSVNAGVTTPVSTYM | M447V | |
| LTNSELLSLINDMPITNDQKKLMSNNVQIVRQQSYS | | |
| IMSIIKEEVLAYVVQLPLYGVIDTPCWKLHTSPLCTT | | |
| NTKEGSNICLTRTDRGWYCDNAGSVSFFPQAETCK | | |
| VQSNRVFCDTMNSLTLPSEVNLCNVDIFNPKYDCKI | | |
| MTSKTDVSSSVITSLGAIVSCYGKTKCTASNKNRGII | | |
| KTFSNGCDYVSNKGVDTVSVGNTLYYVNKQEGKS | | |
| LYVKGEPIINFYDPLVFPSDEFDASISQVNEKINQSR | | |
| EIIRAINIVRKIASEKSAIGGYIPEAPRDGQAYVRKD | | |
| GEWVLLSTFL | | |
| MELLILKANAITTILTAVTFCFASGQNITEEFYQSTCSA | Introduced mutations: | 682 |
| VSKGYLSALRTGWYTCVITIELSNIKENKCNGTDAK | S55C, L188C, S190I, | |
| VKLIKQELDKYKNAVTELQLLMQSTPATNNRARRE | D486S | |
| LPRFMNYTLNNAKKTNVTLSKKRKRRFLGFLLG | Naturally occurring | |
| VGSAIASGVAVSKVLHLEGEVNKIKSALLSTNKAV | substitutions: | |
| VSLSNGVSVCTIKVLDLKNYIDKQLLPIVNKQSCSIS | P102A, I379V, | |
| NIETVIEFQQKNNRLLEITREFSVNAGVTTPVSTYML | M447V | |
| TNSELLSLINDMPITNDQKKLMSNNVQIVRQQSYSI | | |
| MSIIKEEVLAYVVQLPLYGVIDTPCWKLHTSPLCTT | | |
| NTKEGSNICLTRTDRGWYCDNAGSVSFFPQAETCK | | |
| VQSNRVFCDTMNSLTLPSEVNLCNVDIFNPKYDCKI | | |
| MTSKTDVSSSVITSLGAIVSCYGKTKCTASNKNRGII | | |
| KTFSNGCDYVSNKGVDTVSVGNTLYYVNKQEGKS | | |
| LYVKGEPIINFYDPLVFPSSEFDASISQVNEKINQSRE | | |
| IIRAINIVRKIASEKSAIGGYIPEAPRDGQAYVRKD | | |
| GEWVLLSTFL | | |
| MELLILKANAITTILTAVTFCFASGQNITEEFYQSTCSA | Introduced mutations: | 683 |
| VSKGYLSALRTGWYHCVITIELSNIKENKCNGTDA | T54H, S55C, L188C, | |
| KVKLIKQELDKYKNAVTELQLLMQSTPATNNRARR | S190I, D486S | |
| ELPRFMNYTLNNAKKTNVTLSKKRKRRFLGFLL | Naturally occurring | |
| GVGSAIASGVAVSKVLHLEGEVNKIKSALLSTNKA | substitutions: | |
| VVSLSNGVSVCTIKVLDLKNYIDKQLLPIVNKQSCSI | P102A, I379V, | |
| SNIETVIEFQQKNNRLLEITREFSVNAGVTTPVSTYM | M447V | |
| LTNSELLSLINDMPITNDQKKLMSNNVQIVRQQSYS | | |
| IMSIIKEEVLAYVVQLPLYGVIDTPCWKLHTSPLCTT | | |
| NTKEGSNICLTRTDRGWYCDNAGSVSFFPQAETCK | | |
| VQSNRVFCDTMNSLTLPSEVNLCNVDIFNPKYDCKI | | |
| MTSKTDVSSSVITSLGAIVSCYGKTKCTASNKNRGII | | |
| KTFSNGCDYVSNKGVDTVSVGNTLYYVNKQEGKS | | |
| LYVKGEPIINFYDPLVFPSSEFDASISQVNEKINQSRE | | |
| IIRAINIVRKIASEKSAIGGYIPEAPRDGQAYVRKD | | |
| GEWVLLSTFL | | |
| MELLILKANAITTILTAVTFCFASGQNITEEFYQSTCSA | Introduced mutations: | 684 |
| VSKGYLSALRTGWYTSVITIELSNIKENKCNGTDAK | S155C, S190I, S290C, | |
| VKLIKQELDKYKNAVTELQLLMQSTPATNNRARRE | D486S | |
| LPRFMNYTLNNAKKTNVTLSKKRKRRFLGFLLG | Naturally occurring | |
| VGSAIASGVAVCKVLHLEGEVNKIKSALLSTNKAV | substitutions: | |
| VSLSNGVSVLTIKVLDLKNYIDKQLLPIVNKQSCSIS | P102A, I379V, | |
| NIETVIEFQQKNNRLLEITREFSVNAGVTTPVSTYML | M447V | |
| TNSELLSLINDMPITNDQKKLMSNNVQIVRQQSYSI | | |
| MCIIKEEVLAYVVQLPLYGVIDTPCWKLHTSPLCTT | | |
| NTKEGSNICLTRTDRGWYCDNAGSVSFFPQAETCK | | |
| VQSNRVFCDTMNSLTLPSEVNLCNVDIFNPKYDCKI | | |
| MTSKTDVSSSVITSLGAIVSCYGKTKCTASNKNRGII | | |
| KTFSNGCDYVSNKGVDTVSVGNTLYYVNKQEGKS | | |
| LYVKGEPIINFYDPLVFPSSEFDASISQVNEKINQSRE | | |
| IIRAINIVRKIASEKSAIGGYIPEAPRDGQAYVRKD | | |
| GEWVLLSTFL | | |
| MELLILKANAITTILTAVTFCFASGQNITEEFYQSTCSA | Introduced mutations: | 685 |
| VSKGYLSALRTGWYHCVITIELSNIKENKCNGTDA | T54H, S55C, L142C, | |
| KVKLIKQELDKYKNAVTELQLLMQSTPATNNRARR | L188C, V296I, | |
| ELPRFMNYTLNNAKKTNVTLSKKRKRRFLGFLC | N371C, D486S, | |
| GVGSAIASGVAVSKVLHLEGEVNKIKSALLSTNKA | E487Q, D489S | |
| VVSLSNGVSVCTSKVLDLKNYIDKQLLPIVNKQSCS | Naturally occurring | |
| ISNIETVIEFQQKNNRLLEITREFSVNAGVTTPVSTY | substitutions: | |
| MLTNSELLSLINDMPITNDQKKLMSNNVQIVRQQS | P102A, I379V, | |
| YSIMSIIKEEILAYVVQLPLYGVIDTPCWKLHTSPLC | M447V | |
| TTNTKEGSNICLTRTDRGWYCDNAGSVSFFPQAET | | |
| CKVQSNRVFCDTMCSLTLPSEVNLCNVDIFNPKYD | | |
| CKIMTSKTDVSSSVITSLGAIVSCYGKTKCTASNKN | | |
| RGIIKTFSNGCDYVSNKGVDTVSVGNTLYYVNKQE | | |
| GKSLYVKGEPIINFYDPLVFPSSQFSASISQVNEKINQ | | |
| SREIIRAINIVRKIASEKSAIGGYIPEAPRDGQAYVR | | |
| KDGEWVLLSTFL | | |
| MELLILKANAITTILTAVTFCFASGQNITEEFYQSTCSA | Introduced mutations: | 686 |
| VSKGYLSALRTGWYHSVITIELSNIKENKCNGTDAK | T54H, S155C, S190I, | |
| VKLIKQELDKYKNAVTELQLLMQSTPATNNRARRE | S290C, V296I | |
| LPRFMNYTLNNAKKTNVTLSKKRKRRFLGFLLG | Naturally occurring | |
| VGSAIASGVAVCKVLHLEGEVNKIKSALLSTNKAV | substitutions: | |
| VSLSNGVSVLTIKVLDLKNYIDKQLLPIVNKQSCSIS | P102A, I379V, | |
| NIETVIEFQQKNNRLLEITREFSVNAGVTTPVSTYML | M447V | |
| TNSELLSLINDMPITNDQKKLMSNNVQIVRQQSYSI | | |
| MCIIKEEILAYWQLPLYGVIDTPCWKLHTSPLCTTN | | |
| TKEGSNICLTRTDRGWYCDNAGSVSFFPQAETCKV | | |
| QSNRVFCDTMNSLTLPSEVNLCNVDIFNPKYDCKI | | |
| MTSKTDVSSSVITSLGAIVSCYGKTKCTASNKNRGII | | |
| KTFSNGCDYVSNKGVDTVSVGNTLYYVNKQEGKS | | |
| LYVKGEPIINFYDPLVFPSDEFDASISQVNEKINQSR | | |
| EIIRAINIVRKIASEKSAIGGYIPEAPRDGQAYVRKD | | |
| GEWVLLSTFL | | |
| MELLILKANAITTILTAVTFCFASGQNITEEFYQSTCSA | | 687 |
| VSKGYLSALRTGWYHCVITIELSNIKENKCNGTDA | | |
| KVKLIKQELDKYKNAVTELQLLMQSTPATNNRARR | | |
| ELPRFMNYTLNNAKKTNVTLSKKRKRRFLGFLL | | |
| GVGSAIASGVAVSKVLHLEGEVNKIKSALLSTNKA | | |
| VVSLSNGVSVCTSKVLDLKNYIDKQLLPIVNKQSCS | | |
| ISNIETVIEFQQKNNRLLEITREFSVNAGVTTPVSTY | | |
| MLTNSELLSLINDMPITNDQKKLMSNNVQIVRQQS | | |
| YSIMSIIKEEVLAYWQLPLYGVIDTPCWKLHTSPLC | | |
| TTNTKEGSNICLTRTDRGWYCDNAGSVSFFPQAET | | |
| CKVQSNRVFCDTMNSLTLPSEVNLCNVDIFNPKYD | | |
| CKIMTSKTDVSSSVITSLGAIVSCYGKTKCTASNKN | | |
| RGIIKTFSNGCDYVSNKGVDTVSVGNTLYYVNKQE | | |
| GKSLYVKGEPIINFYDPLVFPSSEFDASISQVNEKIN | | |
| QSREIIRAINIVRKIASEKSAIGGYIPEAPRDGQAYV | | |
| RKDGEWVLLSTFL | | |
| MELLILKAAITTILTAVTFCFASGQNITEEFYQSTCSAV | V56C + V164C | 688 |
| SKGYLSALRTGWYTSCITIELSNIKENKCNGTDAVK | | |
| LIKQELDKYKNAVTELQLLMQSTPATNNRARRELP | | |
| RFMYTLNNAKKTVTLSKKRKRRFLGFLLGVGSAI | | |
| ASGVAVSKVLHLEGECNKIKSALLSTNKAVVSLSN | | |
| GVSVLTSKVLDLKNYIDKQLLPIVKQSCSISNIETVIE | | |
| FQQKNNRLLEITREFSVAGVTTPVSTYMLTNSELLS | | |
| LINDMPITNDQKKLMSNNVQIVRQQSYSIMSIIKEEV | | |
| LAYWQLPLYGVIDTPCWKLHTSPLCTTNTKEGSNIC | | |
| LTRTDRGWYCDNAGSVSFFPQAETCKVQSNRVFCD | | |
| TMSLTLPSEVNLCNVDIFNPKYDCKIMTSKTDVSSS | | |
| VITSLGAIVSCYGKTKCTASNKNRGIIKTFSNGCDY | | |
| VSNKGVDTVSVGNTLYYVKQEGKSLYVKGEPIINF | | |
| YDPLVFPSDEFDASISQVEKINQSREIIRAINIVRKIAS | | |
| EKSAIGGYIPEAPRDGQAYVRKDGEWVLLSTFL | | |
| MELLILKAAITTILTAVTFCFASGQNITEEFYQSTCSAV | I57C + S190C | 689 |
| SKGYLSALRTGWYTSVCTIELSNIKENKCNGTDAV | | |
| KLIKQELDKYKNAVTELQLLMQSTPATNNRARREL | | |
| PRFMYTLNNAKKTVTLSKKRKRRFLGFLLGVGS | | |
| AIASGVAVSBVLHLEGEVKIKSALLSTNKAWSLSN | | |
| GVSVLTCBVLDLKNYIDKQLLPIVKQSCSISNIETVI | | |
| EFQQKNNRLLEITREFSVAGVTTPVSTYMLTNSELL | | |
| SLINDMPITNDQKKLMSNNVQIVRQQSYSIMSIIKEE | | |
| VLAYWQLPLYGVIDTPCWKLHTSPLCTTNTKEGSN | | |
| ICLTRTDRGWYCDNAGSVSFFPQAETCKVQSNRVF | | |
| CDTMSLTLPSEVNLCNVDIFNPKYDCKIMTSKTDVS | | |
| SSVITSLGAIVSCYGKTKCTASNKNRGIIKTFSNGCD | | |
| YVSNKGVDTVSVGNTLYYVKQEGKSLYVKGEPIIN | | |
| FYDPLVFPSDEFDASISQVEKINQSREIIRAINIVRKIA | | |
| SEKSAIGGYIPEAPRDGQAYVRKDGEWVLLSTFL | | |
| MELLILKAAITTILTAVTFCFASGQNITEEFYQSTCSAV | T58C + V164C | 690 |
| SKGYLSALRTGWYTSVICIELSNIKENKCNGTDAVK | | |
| LIKQELDKYKNAVTELQLLMQSTPATNNRARRELP | | |
| RFMYTLNNAKKTVTLSKKRKRRFLGFLLGVGSAI | | |
| ASGVAVSKVLHLEGECNKIKSALLSTNKAVVSLSN | | |
| GVSVLTSKVLDLKNYIDKQLLPIVKQSCSISNIETVIE | | |
| FQQKNNRLLEITREFSVAGVTTPVSTYMLTNSELLS | | |
| LINDMPITNDQKKLMSNNVQIVRQQSYSIMSIIKEEV | | |
| LAYWQLPLYGVIDTPCWKLHTSPLCTTNTKEGSNIC | | |
| LTRTDRGWYCDNAGSVSFFPQAETCKVQSNRVFCD | | |
| TMSLTLPSEVNLCNVDIFNPKYDCKIMTSKTDVSSS | | |
| VITSLGAIVSCYGKTKCTASNKNRGIIKTFSNGCDY | | |
| VSNKGVDTVSVGNTLYYVKQEGKSLYVKGEPIINF | | |
| YDPLVFPSDEFDASISQVEKINQSREIIRAINIVRKIAS | | |
| EKSAIGGYIPEAPRDGQAYVRKDGEWVLLSTFL | | |
| MELLILKAAITTILTAVTFCFASGQNITEEFYQSTCSAV | N165C + V296C | 691 |
| SKGYLSALRTGWYTSVITIELSNIKENKCNGTDAVK | | |
| LIKQELDKYKNAVTELQLLMQSTPATNNRARRELP | | |
| RFMYTLNNAKKTVTLSKKRKRRFLGFLLGVGSAI | | |
| ASGVAVSBVLHLEGEVCKIKSALLSTNKAWSLSNG | | |
| VSVLTSBVLDLKNYIDKQLLPIVKQSCSISNIETVIEF | | |
| QQKNNRLLEITREFSVAGVTTPVSTYMLTNSELLSLI | | |
| NDMPITNDQKKLMSNNVQIVRQQSYSIMSIIKEECL | | |
| AYWQLPLYGVIDTPCWKLHTSPLCTTNTKEGSNICL | | |
| TRTDRGWYCDNAGSVSFFPQAETCKVQSNRVFCDT | | |
| MSLTLPSEVNLCNVDIFNPKYDCKIMTSKTDVSSSV | | |
| ITSLGAIVSCYGKTKCTASNKNRGIIKTFSNGCDYVS | | |
| NKGVDTVSVGNTLYYVKQEGKSLYVKGEPIINFYD | | |
| PLVFPSDEFDASISQVEKINQSREIIRAINIVRKIASEK | | |
| SAIGGYIPEAPRDGQAYVRKDGEWVLLSTFL | | |
| MELLILKAAITTILTAVTFCFASGQNITEEFYQSTCSAV | K168C + V296C | 692 |
| SKGYLSALRTGWYTSVITIELSNIKENKCNGTDAKV | | |
| KLIKQELDKYKNAVTELQLLMQSTPATIWRARREL | | |
| PRFMYTLAKKTVTLSKKRKRRFLGFLLGVGSAIA | | |
| SGVAVSBVLHLEGEVKICSALLSTNKAWSLSNGVS | | |
| VLTSBVLDLKNYIDKQLLPIVKQSCSISNIETVIEFQQ | | |
| KNNRLLEITREFSVAGVTTPVSTYMLTNSELLSLIND | | |
| MPITNDQKKLMSNNVQIVRQQSYSIMSIIKEECLAY | | |
| WQLPLYGVIDTPCWKLHTSPLCTTNTKEGSNICLTR | | |
| TDRGWYCDNAGSVSFFPQAETCKVQSNRVFCDTM | | |
| SLTLPSEVNLCNVDIFNPKYDCKIMTSKTDVSSSVIT | | |
| SLGAIVSCYGKTKCTASNKNRGIIKTFSNGCDYVSN | | |
| KGVDTVSVGNTLYYVKQEGKSLYVKGEPIINFYDP | | |
| LVFPSDEFDASISQVEKINQSREIIRAINIVRKIASEKS | | |
| AIGGYIPEAPRDGQAYVRKDGEWVLLSTFL | | |
| MELLILKAAITTILTAVTFCFASGQNITEEFYQSTCSAV | M396C + F483C | 693 |
| SKGYLSALRTGWYTSVITIELSNIKENKCNGTDAVK | | |
| LIKQELDKYKNAVTELQLLMQSTPATNNRARRELP | | |
| RFMYTLNNAKKTVTLSKKRKRRFLGFLLGVGSAI | | |
| ASGVAVSKVLHLEGEVKIKSALLSTNKAVVSLSNG | | |
| VSVLTSKVLDLKNYIDKQLLPIVKQSCSISNIETVIEF | | |
| QQKNNRLLEITREFSVAGVTTPVSTYMLTNSELLSLI | | |
| NDMPITNDQKKLMSNNVQIVRQQSYSIMSIIKEEVL | | |
| AYWQLPLYGVIDTPCWKLHTSPLCTTNTKEGSNICL | | |
| TRTDRGWYCDNAGSVSFFPQAETCKVQSNRVFCDT | | |
| MSLTLPSEVNLCNVDIFNPKYDCKICTSKTDVSSSVI | | |
| TSLGAIVSCYGKTKCTASNKNRGIIKTFSNGCDYVS | | |
| NKGVDTVSVGNTLYYVKQEGKSLYVKGEPIINFYD | | |
| PLVCPSDEFDASISQVEKINQSREIIRAINIVRKIASEK | | |
| SAIGGYIPEAPRDGQAYVRKDGEWVLLSTFL | | |
| METPAQLLFLLLLWLPDTTGFASGQNITEEFYQSTC | Ectodomain + Igk | 694 |
| SAVSKGYLSALRTGWYTSVITIELSNIKKNKCNGTD | signal + foldon | |
| AKVKLIKQELDKYKNAVTELQLLMQSTQATNNRA | | |
| RRELPRFMNYTLNNAKKTNVTLSKKRKRRFLGF | | |
| LLGVGSAIASGVAVSKVLHLEGEVNKIKSALLSTNK | | |
| AVVSLSNGVSVLTSKVLDLKNYIDKQLLPIVNKQSC | | |
| SISNIETVIEFQQKNNRLLEITREFSVNAGVTTPVSTY | | |
| MLTNSELLSLINDMPITNDQKKLMSNNVQIVRQQS | | |
| YSIMSIIKEEVLAYVVQLPLYGVIDTPCWKLHTSPLC | | |
| TTNTKEGSNICLTRTDRGWYCDNAGSVSFFPQAET | | |
| CKVQSNRVFCDTMNSLTLPSEVNLCNVDIFNPKYD | | |
| CKIMTSKTDVSSSVITSLGAIVSCYGKTKCTASNKN | | |
| RGIIKTFSNGCDYVSNKGVDTVSVGNTLYYVNKQE | | |
| GKSLYVKGEPIINFYDPLVFPSDEFDASISQVNEKIN | | |
| QSREIIRAINIVRKIASEKSAIGGYIPEAPRDGQAYV | | |
| RKDGEWVLLSTFL | | |
| METPAQLLFLLLLWLPDTTGFASGQNITEEFYQSTC | Ectodomain + Igk | 695 |
| SAVSKGYLSALRTGWYTSVITIELSNIKENKCNGTD | signal + foldon | |
| AKVKLIKQELDKYKNAVTELQLLMQSTPATNNRA | | |
| RRELPRFMNYTLNNAKKTNVTLSKKRKRRFLGF | | |
| LLGVGSAIASGVAVCKVLHLEGEVNKIKSALLSTN | | |
| KAVVSLSNGVSVLTFKVLDLKNYIDKQLLPILNKQS | | |
| CSISNIETVIEFQQKNNRLLEITREFSVNAGVTTPVST | | |
| YMLTNSELLSLINDMPITNDQKKLMSNNVQIVRQQ | | |
| SYSIMCIIKEEVLAYVVQLPLYGVIDTPCWKLHTSPL | | |
| CTTNTKEGSNICLTRTDRGWYCDNAGSVSFFPQAE | | |
| TCKVQSNRVFCDTMNSLTLPSEVNLCNVDIFNPKY | | |
| DCKIMTSKTDVSSSVITSLGAIVSCYGKTKCTASNK | | |
| NRGIIKTFSNGCDYVSNKGVDTVSVGNTLYYVNKQ | | |
| EGKSLYVKGEPIINFYDPLVFPSDEFDASISQVNEKI | | |
| NQSREIIRAINIVRKIASEKSAIGGYIPEAPRDGQAY | | |
| VRKDGEWVLLSTFL | | |
| MELLILKANAITTILTAVTFCFASGQNITEEFYQSTCSA | | 696 |
| VSKGYLSALRTGWYTSVITIELSNIKKNKCNGTDAK | | |
| VKLIKQELDKYKNAVTELQLLMQSTQATNNRARR | | |
| ELPRFMNYTLNNAKKTNVTLSKKRKRRFLGFLL | | |
| GVGSAIASGVAVSKVLHLEGEVNKIKSALLSTNKA | | |
| VVSLSNGVSVLTSKVLDLKNYIDKQLLPIVNKQSCS | | |
| ISNIETVIEFQQKNNRLLEITREFSVNAGVTTPVSTY | | |
| MLTNSELLSLINDMPITNDQKKLMSNNVQIVRQQS | | |
| YSIMSIIKEEVLAYVVQLPLYGVIDTPCWKLHTSPLC | | |
| TTNTKEGSNICLTRTDRGWYCDNAGSVSFFPQAET | | |
| CKVQSNRVFCDTMNSLTLPSEVNLCNVDIFNPKYD | | |
| CKIMTSKTDVSSSVITSLGAIVSCYGKTKCTASNKN | | |
| RGIIKTFSNGCDYVSNKGVDTVSVGNTLYYVNKQE | | |
| GKSLYVKGEPIINFYDPLVFPSDEFDASISQVNEKIN | | |
| QSREIIRAINIVRKIASEKSAIGGYIPEAPRDGQAYV | | |
| RKDGEWVLLSTFL | | |
| MELLILKANAITTILTAVTFCFASGQNITEEFYQSTCSA | S155C, S290C, S190F, | 697 |
| VSKGYLSALRTGWYTSVITIELSNIKENKCNGTDAK | V207L | |
| VKLIKQELDKYKNAVTELQLLMQSTPATNNRARRE | | |
| LPRFMNYTLNNAKKTNVTLSKKRKRRFLGFLLG | | |
| VGSAIASGVAVCKVLHLEGEVNKIKSALLSTNKAV | | |
| VSLSNGVSVLTFKVLDLKNYIDKQLLPILNKQSCSIS | | |
| NIETVIEFQQKNNRLLEITREFSVNAGVTTPVSTYML | | |
| TNSELLSLINDMPITNDQKKLMSNNVQIVRQQSYSI | | |
| MCIIKEEVLAYVVQLPLYGVIDTPCWKLHTSPLCTT | | |
| NTKEGSNICLTRTDRGWYCDNAGSVSFFPQAETCK | | |
| VQSNRVFCDTMNSLTLPSEVNLCNVDIFNPKYDCKI | | |
| MTSKTDVSSSVITSLGAIVSCYGKTKCTASNKNRGII | | |
| KTFSNGCDYVSNKGVDTVSVGNTLYYVNKQEGKS | | |
| LYVKGEPIINFYDPLVFPSDEFDASISQVNEKINQSR | | |
| EIIRAINIVRKIASEKSAIGGYIPEAPRDGQAYVRKD | | |
| GEWVLLSTFL | | |
| MELLILKANAITTILTAVTFCFASGQNITEEFYQSTC | Deletion of p27 | 698 |
| SAVSKGYLSALRTGWYTSVITIELSNIKKNKCNGTD | sequence | |
| AKVKLIKQELDKYKNAVTELQLLMQSTQATNNRA | | |
| RQQQQRFLGFLLGVGSAIASGVAVSKVLHLEGEVN | | |
| KIKSALLSTNKAVVSLSNGVSVLTSKVLDLKNYIDK | | |
| QLLPIVNKQSCSISNIETVIEFQQKNNRLLEITREFSV | | |
| NAGVTTPVSTYMLTNSELLSLINDMPITNDQKKLM | | |
| SNNVQIVRQQSYSIMSIIKEEVLAYVVQLPLYGVIDT | | |
| PCWKLHTSPLCTTNTKEGSNICLTRTDRGWYCDNA | | |
| GSVSFFPQAETCKVQSNRVFCDTMNSLTLPSEVNLC | | |
| NVDIFNPKYDCKIMTSKTDVSSSVITSLGAIVSCYGK | | |
| TKCTASNKNRGIIKTFSNGCDYVSNKGVDTVSVGN | | |
| TLYYVNKQEGKSLYVKGEPIINFYDPLVFPSDEFDA | | |
| SISQVNEKINQSREIIRAINIVRKIASEKSAIGGYIPEA | | |
| PRDGQAYVRKDGEWVLLSTFL | | |
| MELLILKTNAITAILAAVTLCFASSQNITEEFYQSTC | Deletion of p27 | 699 |
| SAVSKGYLSALRTGWYTSVITIELSNIKENKCNGTD | sequence | |
| AKVKLIKQELDKYKSAVTELQLLMQSTPATNNKFL | | |
| GFLLGVGSAIASGIAVSKVLHLEGEVNKIKSALLST | | |
| NKAVVSLSNGVSVLTSKVLDLKNYIDKQLLPIVNK | | |
| QSCSISNIETVIEFQQKNNRLLEITREFSVNAGVTTPV | | |
| STYMLTNSELLSLINDMPITNDQKKLMSNNVQIVRQ | | |
| QSYSIMSIIKEEVLAYVVQLPLYGVIDTPCWKLHTSP | | |
| LCTTNTKEGSNICLTRTDRGWYCDNAGSVSFFPLAE | | |
| TCKVQSNRVFCDTMNSLTLPSEVNLCNIDIFNPKYD | | |
| CKIMTSKTDVSSSVITSLGAIVSCYGKTKCTASNKN | | |
| RGIIKTFSNGCDYVSNKGVDTVSVGNTLYYVNKQE | | |
| GKSLYVKGEPIINFYDPLVFPSDEFDASISQVNEKIN | | |
| QSREIIRAINIVRKIASEKSAIGGYIPEAPRDGQAYV | | |
| RKDGEWVLLSTFL | | |
| MELLILKANAITTILTAVTFCFASGQNITEEFYQSTC | | 700 |
| SAVSKGYLSALRTGWYTSVITIELSNIKENKCNGTD | | |
| AKVKLIKQELDKYKNAVTELQLLMQSTPATNNRA | | |
| RRELPRFMNYTLNNAKKTNVTLSKKRKRRFLGF | | |
| LLGVGSAIASGVAVCKVLHLEGEVNKIKSALLSTN | | |
| KAVVSLSNGVSVLTFKVLDLKNYIDKQLLPILNKQS | | |
| CSISNIETVIEFQQKNNRLLEITREFSVNAGVTTPVST | | |
| YMLTNSELLSLINDMPITNDQKKLMSNNVQIVRQQ | | |
| SYSIMCIIKEEVLAYVVQLPLYGVIDTPCWKLHTSPL | | |
| CTTNTKEGSNICLTRTDRGWYCDNAGSVSFFPQAE | | |
| TCKVQSNRVFCDTMNSLTLPSEVNLCNVDIFNPKY | | |
| DCKIMTSKTDVSSSVITSLGAIVSCYGKTKCTASNK | | |
| NRGIIKTFSNGCDYVSNKGVDTVSVGNTLYYVNKQ | | |
| EGKSLYVKGEPIINFYDPLVFPSDEFDASISQVNEKI | | |
| NQSREIIRAINIVRKIASEKSAIGGYIPEAPRDGQAY | | |
| VRKDGEWVLLSTFL | | |
| MELLILKANAITTILTAVTFCFASQNITEEFYQSTCS | | |
| AVSKGYLSALRTGWYTSVITIELSNIKENKCNGTDA | | 701 |
| KVKLIKQELDKYKNAVTELQLLMQSTPATNNRARR | | |
| ELPRFMNYTLNNAKKINVILSKKRKRRFLGFLLG | | |
| VGSAIASGVAVCKVLHLEGEVNKIKSALLSINKAVV | | |
| SLSNGVSVLIFKVLDLKNYIDKQLLPILNKQSCSISNI | | |
| ETVIEFQQKNNRLLEITREFSVNAGVITPVSTYMLIN | | |
| SELLSLINDMPITNDQKKLMSNNVQIVRQQSYSIMC | | |
| IIKEEVLAYVVQLPLYGVIDTPCWKLHISPLCTINTK | | |
| EGSNICLTRIDRGWYCDNAGSVSFFPQAETCKVQSN | | |
| RVFCDTMNSLTLPSEVNLCNVDIFNPKYDCKIMISK | | |
| TDVSSSVITSLGAIVSCYGKTKCIASNKNRGIIKTFSN | | |
| GCDYVSNKGVDTVSVGNTLYYVNKQEGKSLYVKG | | |
| EPIINFYDPLVFPSDEFDASISQVNEKINQSREIIRAINI | | |
| VRKIASEKSAIGGYIPEAPRDGQAYVRKDGEWVL | | |
| LSTFL | | |

In some embodiments, the recombinant polypeptide comprises an engineered ectodomain of a Respiratory Syncytial Virus (RSV) fusion (F) protein, wherein the ectodomain comprises (a) a C-terminal helix-forming segment, between about residue 500 and about residue 530 relative to SEQ ID NO: 1, comprising one or more amino acid substitutions selected such that the segment forms a stable alpha-helical homotrimer, (b) one, two, three or more amino acid substitutions at positions 140, 399, 400, 485, 486, 487, 488, 489, 494, or 498 relative to SEQ ID NO: 1, (c) one, two, three or more amino acid substitutions at positions 56, 58, 154, 187, 296, or 298 relative to SEQ ID NO: 1, (d) one, two, three or more amino acid substitutions at positions 75, 216, 218, or 219 relative to SEQ ID NO: 1, (e) one, two, three or more amino acid substitutions at positions 92, 232, 235, 238, 249, 250, or 254 relative to SEQ ID NO: 1, (f) one, two, three or more amino acid substitutions at positions 67, 137, or 339 relative to SEQ ID NO: 1, (g) a substitution of a non-cleavable linker in place of a furin cleavage site at about residue 100 to about residue 140 relative to SEQ ID NO: 1 or (h) any combination of (a)-(g).

In some embodiments, the ectodomain comprises the C-terminal helix-forming segment, between about residue 500 and about residue 530 relative to SEQ ID NO: 1, comprising one or more amino acid substitutions selected such that the segment forms a stable alpha-helical homotrimer. In some embodiments, the C-terminal helix-forming segment comprises between about 10 and about 30 residues.

In some embodiments, the C-terminal helix-forming segment comprises substitutions relative to the reference sequence SEQ ID NO: 1 at two or more, three or more, or four or more residues that generate hydrophobic contacts between the segments in the alpha-helical homotrimer.

In some embodiments, the C-terminal helix-forming segment comprises (1) an amino acid substitution at position F505 relative to SEQ ID NO: 1, wherein F is substituted with A, I, L, M, V, G, T; (2) an amino acid substitution at position I506 relative to SEQ ID NO: 1, wherein I is substituted with any amino acids except P, preferably D, E, K, N, Q, R, S, T, Y or A, I, L, V; (3) an amino acid substitution at position R507 relative to SEQ ID NO: 1, wherein R is substituted with any amino acids except P, preferably D, E, K, N, Q, R, S, T, Y or A, I, L, V; (4) an amino acid substitution at position K508 relative to SEQ ID NO: 1, wherein R is substituted with K, Q, R, preferably A, V, T, I; (5) an amino acid substitution at position S509 relative to SEQ ID NO: 1, wherein S is substituted with A, I, L, M, V, F, W, Y, G, T, preferably A, I, L, M, V; (6) an amino acid substitution at position D510 relative to SEQ ID NO: 1, wherein D is substituted with any amino acids, preferably D, E, K, N, Q, R, S, T, Y; (7) an amino acid substitution at position E511 relative to SEQ ID NO: 1, wherein E is substituted with any amino acids; (8) an amino acid substitution at position L512 relative to SEQ ID NO: 1, wherein L is substituted with D, E, K, N, Q, R, S, T, Y, preferably A, I, L, M, V, F, W, Y, G, T; (9) an amino acid substitution at position L513 relative to SEQ ID NO: 1, wherein L is substituted with any amino acids, preferably A, I, L, M, V, F, W, Y, G, more preferably D, E, K, N, Q, R, S, T, Y; (10) an amino acid substitution at position H514 relative to SEQ ID NO: 1, wherein H is substituted with any amino acids except P, preferably D, E, K, N, Q, R, S, T, Y; (11) an amino acid substitution at position N515 relative to SEQ ID NO: 1, wherein N is substituted with any amino acids except P, preferably A, I, L, M, V, F, W, Y, G; (12) an amino acid substitution at position V516 relative to SEQ ID NO: 1, wherein V is substituted with A, I, L, M, V, F, W, Y, G, or T, S, K; (13) an amino acid substitution at position N517 relative to SEQ ID NO: 1, wherein N is substituted with any amino acid except P, preferably D, E, K, N, Q, R, S, T, Y; (14) an amino acid substitution at position T518 relative to SEQ ID NO: 1, wherein Tis substituted with any amino acid except P, preferably D, E, K, N, Q, R, S, T, Y; (15) an amino acid substitution at position G519 relative to SEQ ID NO: 1, wherein G is substituted with any amino acid except P, preferably D, E, K, N, Q, R, S, T, Y; and/or (16) any combination of (1)-(15).

In some embodiments, the segment comprises (1) an amino acid substitution at position L503 relative to SEQ ID NO: 1, wherein F is substituted with Q, V, K, R, N, L, (2) an amino acid substitution at position A504 relative to SEQ ID NO: 1, wherein I is substituted with any amino acids except P, preferably S, T, L, A, Q, K, E, Y, (3) an amino acid substitution at position F505 relative to SEQ ID NO: 1, wherein F is substituted with I, V, N, T, L, (4) an amino acid substitution at position I506 relative to SEQ ID NO: 1, wherein I is substituted with any amino acids except P, preferably Q, N, K, R, V, S, (5) an amino acid substitution at position R507 relative to SEQ ID NO: 1, wherein R is substituted with any amino acids except P, preferably A, N, K, E, D, Q, (6) an amino acid substitution at position K508 relative to SEQ ID NO: 1, wherein R is substituted with T, M, V, R, (7) an amino acid substitution at position S509 relative to SEQ ID NO: 1, wherein S is substituted with T, I, K, Q, M, E, V, S, (8) an amino acid substitution at position D510 relative to SEQ ID NO: 1, wherein D is substituted with S, K, N, D, E, (9) an amino acid substitution at position E511 relative to SEQ ID NO: 1, wherein E is substituted with R, S, E, K, A, T, L, (10) an amino acid substitution at position L512 relative to SEQ ID NO: 1, wherein L is substituted with V, N, T, L, (11) an amino acid substitution at position L513 relative to SEQ ID NO: 1, wherein L is substituted with D, T, H, K, E, N, R, (12) an amino acid substitution at position H514 relative to SEQ ID NO: 1, wherein H is substituted with A, N, E, S, V, K, T, D, (13) an amino acid substitution at position N515 relative to SEQ ID NO: 1, wherein N is substituted with I, E, L, T, Q, (14) an amino acid substitution at position V516 relative to SEQ ID NO: 1, wherein V is substituted with E, I, K, N, R, Q, (15) an amino acid substitution at position N517 relative to SEQ ID NO: 1, wherein N is substituted with A, S, K, E, R, (16) an amino acid substitution at position T518 relative to SEQ ID NO: 1, wherein T is substituted with K, S, Q, R, D, E, (17) an amino acid substitution at position G519 relative to SEQ ID NO: 1, wherein G is substituted with V, L, I, (18) an amino acid substitution at position I520 relative to SEQ ID NO: 1, wherein G is substituted with K, Q, E, N, T, (19) an amino acid substitution at position P521 relative to SEQ ID NO: 1, wherein G is substituted with H, D, E, K, R, N, Q, (20) an amino acid substitution at position E522 relative to SEQ ID NO: 1, wherein G is substituted with L, R, I, V, (21) an amino acid substitution at position A523 relative to SEQ ID NO: 1, wherein G is substituted with E, V, L, K, R I, (22) an amino acid substitution at position P524 relative to SEQ ID NO: 1, wherein G is substituted with A, K, T, E, R, (23) an amino acid substitution at position R525 relative to SEQ ID NO: 1, wherein G is substituted with H, R, S, L, N, E, D, (24) an amino acid substitution at position D526 relative to SEQ ID NO: 1, wherein G is substituted with I, L, V, R, (25) an amino acid substitution at position G527 relative to SEQ ID NO: 1, wherein G is substituted with E, K, Q, D, (26) an amino acid substitution at position Q528 relative to SEQ ID NO: 1, wherein G is substituted with D, K, S, R, A, (27) an amino acid substitution at position A529 relative to SEQ ID NO: 1, wherein G is substituted with T, L, (28) an amino acid substitution at position Y530 relative to SEQ ID NO: 1, wherein G is substituted with L, E, T, (29) an amino acid substitution at position V531 relative to SEQ ID NO: 1, wherein G is substituted with A, R, K, (30) an amino acid substitution at position R532 relative to SEQ ID NO: 1, wherein G is substituted with V, A, and/or (31) any combination of (1)-(30).

In some embodiments, the segment comprises a polypeptide sequence listed in Table 2B or Table 2C, or a polypeptide sequence having between 1 and 5 amino acid substitutions thereto. In some embodiments, the segment comprises the polypeptide sequence NQSREIIRAINIVRKIASEK (SEQ ID NO: 10), NQSALWLEAAKYVKQAREKS (SEQ ID NO: 11), NQSAKNAEAAKIAEETKRKD (SEQ ID NO: 12), or NQSRETAKAVSAVK (SEQ ID NO: 75), or a polypeptide sequence having between 1 and 5 amino acid substitutions thereto. In some embodiments, the segment comprises the polypeptide sequence NQSREIIRAINIVRKIASEK (SEQ ID NO: 10) or a polypeptide sequence having between 1 and 5 amino acid substitutions thereto. In some embodiments, the segment comprises the polypeptide sequence NQSREIIRAINIVRKIASEK (SEQ ID NO: 10).

In some embodiments, the ectodomain comprises one, two, three or more amino acid substitutions at positions 140, 399, 400, 485, 486, 487, 488, 489, 494, or 498 relative to SEQ ID NO: 1. In some embodiments, the ectodomain comprises one or more of the following sets of amino acid substitutions relative to SEQ ID NO: 1: E487R+K498A, E487R+K498E, E487K+K498E, D486A+E487R+K498A, D486Q+E487R+K498A, D486E+E487A+D489A+T400D, D486A+E487M+K498A, E487Q, D486S, F488W+D489A+T400D+E487R+K498A, F140W+D489A+T400D+E487R+K498A, Q494I+S485I+K399A+487R+498A, Q494M+S485I+K399A; D486A+487M+498A, Q494L+S485A+K399V+D486A+487M+498A, Q494M+S485A+K399V+D486A+487M+498A, Q494A+S485F+K399V+D486A+487M+498Y, D489A+T400D+E487R+K498A, or D489A+T400D. In some embodiments, the ectodomain comprises the amino acid substitutions D489A, T400D, E487R, and K498A.

In some embodiments, the ectodomain comprises the amino acid substitutions F488W, D489A, T400D, E487R, K498A, and D486A. In some embodiments, the ectodomain comprises the amino acid substitutions F488W, D489A, T400D, E487R, K498A, and T249P.

In some embodiments, the polypeptide comprises, C-terminal to the ectodomain, a heterologous multimerization domain. In some embodiments, the multimerization domain is a trimerization domain. In some embodiments, the multimerization domain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64). In some embodiments, the ectodomain comprises the amino acid substitutions S155C, S290C, S190F, and V207L.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 6, below, optionally lacking a p27 peptide shown in bold, and in which “X” refers to sites involving an added C-terminal helical segment and can be any amino acid:

(SEQ ID NO: 6)
QNITEEFYQSTCSAVSRGYLSALRTGWYTSVITIELSNIKETKCNGTDT
KVKLIKQELDKYKNAVTELQLLMQNTPAVNNRARREAPQYMNYTINTTK
NLNVSISKKRKRRFLGFLLGVGSAIASGIAVCKVLHLEGEVNKIKNALQ
LTNKAVVSLSNGVSVLTFRVLDLKNYINNQLLPMLNRQSCRISNIETVI
EFQQKNSRLLEITREFSVNAGVTTPLSTYMLINSELLSLINDMPITNDQ
KKLMSSNVQIVRQQSYSIMCIIKEEVLAYVVQLPIYGVIDTPCWKLHTS
PLCTTNIKEGSNICLTRTDRGWYCDNAGSVSFFPQADTCKVQSNRVFCD
TMNSLTLPSEVSLCNTDIFNSKYDCKIMTSKTDISSSVITSLGAIVSCY
GKTKCTASNKNRGIIKTFSNGCDYVSNKGVDTVSVGNTLYYVNKLEGKN
LYVKGEPIINYYDPLVFPSDEFDASISQVNEKINQSXXXXXXXXXXXXX
XXXX.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 7, below, optionally lacking a p27 peptide shown in bold, in which “X” refers to sites involving an added C-terminal helical segment and can be any amino acid:

(SEQ ID NO: 7)
QNITEEFYQSTCSAVSKGYLSALRTGWYTSVITIELSNIKENKCNGTDA
KVKLIKQELDKYKNAVTELQLLMQSTPATNNRARRELPRFMNYTLNNAK
KTNVTLSKKRKRRFLGFLLGVGSAIASGVAVCKVLHLEGEVNKIKSALL
STNKAVVSLSNGVSVLTFKVLDLKNYIDKQLLPILNKQSCSISNIETVI
EFQQKNNRLLEITREFSVNAGVTTPVSTYMLINSELLSLINDMPITNDQ
KKLMSNNVQIVRQQSYSIMCIIKEEVLAYVVQLPLYGVIDTPCWKLHTS
PLCTTNTKEGSNICLTRTDRGWYCDNAGSVSFFPQAETCKVQSNRVFCD
TMNSLTLPSEVNLCNVDIFNPKYDCKIMTSKTDVSSSVITSLGAIVSCY
GKTKCTASNKNRGIIKTFSNGCDYVSNKGVDTVSVGNTLYYVNKQEGKS
LYVKGEPIINFYDPLVFPSDEFDASISQVNEKINQSXXXXXXXXXXXXX
XXXX.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 8, below, optionally lacking a p27 peptide shown in bold:

(SEQ ID NO: 8)
QNITEEFYQSTCSAVSRGYLSALRTGWYTSVITIELSNIKETKCNGTDT
KVKLIKQELDKYKNAVTELQLLMQNTPAVNNRARREAPQYMNYTINTTK
NLNVSISKKRKRRFLGFLLGVGSAIASGIAVCKVLHLEGEVNKIKNALQ
LTNKAVVSLSNGVSVLTFRVLDLKNYINNQLLPMLNRQSCRISNIETVI
EFQQKNSRLLEITREFSVNAGVTTPLSTYMLINSELLSLINDMPITNDQ
KKLMSSNVQIVRQQSYSIMCIIKEEVLAYVVQLPIYGVIDTPCWKLHTS
PLCTTNIKEGSNICLTRTDRGWYCDNAGSVSFFPQADTCKVQSNRVFCD
TMNSLTLPSEVSLCNTDIFNSKYDCKIMTSKTDISSSVITSLGAIVSCY
GKTKCTASNKNRGIIKTFSNGCDYVSNKGVDTVSVGNTLYYVNKLEGKN
LYVKGEPIINYYDPLVFPSDEFDASISQVNEKINQSREIIRAINIVRKI
ASEK.

In some embodiments, the ectodomain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 9, below, optionally lacking a p27 peptide shown in bold:

(SEQ ID NO: 9)
QNITEEFYQSTCSAVSKGYLSALRTGWYTSVITIELSNIKENKCNGTDA
KVKLIKQELDKYKNAVTELQLLMQSTPATNNRARRELPRFMNYTLNNAK
KTNVTLSKKRKRRFLGFLLGVGSAIASGVAVCKVLHLEGEVNKIKSALL
STNKAVVSLSNGVSVLTFKVLDLKNYIDKQLLPILNKQSCSISNIETVI
EFQQKNNRLLEITREFSVNAGVTTPVSTYMLINSELLSLINDMPITNDQ
KKLMSNNVQIVRQQSYSIMCIIKEEVLAYVVQLPLYGVIDTPCWKLHTS
PLCTINTKEGSNICLTRTDRGWYCDNAGSVSFFPQAETCKVQSNRVFCD
TMNSLTLPSEVNLCNVDIFNPKYDCKIMTSKTDVSSSVITSLGAIVSCY
GKTKCTASNKNRGIIKTFSNGCDYVSNKGVDTVSVGNTLYYVNKQEGKS
LYVKGEPIINFYDPLVFPSDEFDASISQVNEKINQSREIIRAINIVRKI
ASEK.

In some embodiments, the polypeptide comprises a sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 1-9.

In another aspect, the disclosure provides a trimeric protein complex comprising a polypeptide disclosed herein. In some embodiments, the thermal stability, assayed by nanoDSF, is increased by at least 10° C., at least 15° C., at least 20° C., about 10° C. to about 30° C., about 10° C. to about 20° C., or about 20° C. to about 30° C. compared to a trimeric protein complex lacking modifications (a)-(h). In some embodiments, the stability, assayed by storage at about 40° C., is increased compared to a trimeric protein complex lacking modifications (a)-(h). In some embodiments, the thermal stability is increased compared to a reference RSV F protein comprising amino acid substitutions consisting essentially of S155C, S290C, S190F, and V207L (DS-Cav1).

In another aspect, the disclosure provides a protein nanostructure comprising a trimeric component comprising a polypeptide disclosed herein. In some embodiments, the nanostructure is a two-component nanostructure comprising the first, trimeric component and a second, pentameric component. In some embodiments, the first, trimeric component comprises an engineered ectodomain of a Respiratory Syncytial Virus (RSV) fusion (F) polypeptide and an I53-50A polypeptide. In some embodiments, the first, trimeric component comprises a fusion protein comprising, in N- to C-terminal order, the RSV fusion (F) polypeptide, an amino acid linker, and the I53-50A polypeptide. In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, and V207L relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, D489A, T400D, E487R, and K498A relative to SEQ ID NO: 1 and a C-terminal helix-forming comprising segment the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, F488W, D489A, T400D, E487R, K498A, and T249P relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the nanostructure is a two-component nanostructure comprising the first, trimeric component, wherein the first trimeric component comprises an engineered ectodomain of a RSV F polypeptide comprising an amino acid substitutions at position S155C, S290C, S190F, V207L, F488W, D489A, T400D, E487R, K498A, and D486A relative to SEQ ID NO: 1 and a C-terminal helix-forming segment comprising the polypeptide sequence NQSREIIRAINIVRKIASEK (SEQ ID NO: 10), and a multimerization domain comprising a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64), and/or the second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

In some embodiments, the trimeric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of the sequences listed in Table 19. In some embodiments, the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 20, 44, 45, 52, 71, 73, 74.

In another aspect, the disclosure provides a recombinant polypeptide, comprising an alpha-helical segment and a multimerization domain, wherein the segment comprises a polypeptide sequence listed in Table 25A or Table 25B, or a polypeptide sequence having between 1 and 5 amino acid substitutions thereto. In some embodiments, the polypeptide comprises a trimeric pathogen protein, N-terminally or C-terminally linked to the alpha-helical segment. In some embodiments, the segment comprises the polypeptide sequence NQSREIIRAINIVRKIASEK (SEQ ID NO: 10), NQSALWLEAAKYVKQAREKS (SEQ ID NO: 11), NQSAKNAEAAKIAEETKRKD (SEQ ID NO: 12), or NQSRETAKAVSAVK (SEQ ID NO: 75), or a polypeptide sequence having between 1 and 5 amino acid substitutions thereto. In some embodiments, polypeptide comprises, N-terminal to the segment, an antigen.

Human Metapneumovirus (hMPV)

hMPV is a negative-sense, single-stranded RNA virus causing upper and lower respiratory disease. hMPV shares substantial homology with respiratory syncytial virus (RSV) in its surface glycoproteins. F protein, existing as trimers, is a type I glycoprotein.

Illustrative sequences are shown in Table 6A. A native hMPV F protein sequence was used for design. The signal peptide is underlined with italic

TABLE 6A

| | | | SEQ |
|---|---|---|---|
| | | | ID |
| | Description | Sequence | NO: |
| hMPV | Reference | MSWKVVIIFSLLITPQHGLKESYLEESCSTITEGY | 104 |
| F protein | sequence | LSVLRTGWYTNVFTLEVGDVENLTCADGPSLIKTE | |
| | | LDLTKSALRELRTVSADQLAREEQIENPRQSRFVL | |
| | | GAIALGVATAAAVTAGVAIAKTIRLESEVTAIKNA | |
| | | LKKTNEAVSTLGNGVRVLATAVRELKDFVSKNLTR | |
| | | AINKNKCDIADLKMAVSFSQFNRRFLNVVRQFSDN | |
| | | AGITPAISLDLMTDAELARAVSNMPTSAGQIKLML | |
| | | ENRAMVRRKGFGILIGVYGSSVIYMVQLPIFGVID | |
| | | TPCWIVKAAPSCSEKKGNYACLLREDQGWYCQNAG | |
| | | STVYYPNEKDCETRGDHVFCDTAAGINVAEQSKEC | |
| | | NINISTTNYPCKVSTGRHPISMVALSPLGALVACY | |
| | | KGVSCSIGSNRVGIIKQLNKGCSYITNQDADTVTI | |
| | | DNTVYQLSKVEGEQHVIKGRPVSSSFDPVKFPEDQ | |
| | | FNVALDQVFESIENSQALVDQSNRILSSAEKGNTG | |
| | | FIIVIILTAVLGSTMILVSVFIIIKKTKKPTGAPP | |
| | | ELSGV | |
| hMPV | GenBank: | MSWKVVIIFSLLITPQHGLKESYLEESCSTITEGY | 179 |
| F protein | AY145297 | LSVLRTGWYTNVFTLEVGDVENLTCSDGPSLIKTE | |
| | | LDLTKSALRELKTVSADQLAREEQIENPRQSRFVL | |
| | | GAIALGVATAAAVTAGVAIAKTIRLESEVTAIKNA | |
| | | LKTTNEAVSTLGNGVRVLATAVRELKDFVSKNLTR | |
| | | AINKNKCDIDDLKMAVSFSQFNRRFLNVVRQFSDN | |
| | | AGITPAISLDLMTDAELARAVSNMPTSAGQIKLML | |
| | | ENRAMVRRKGFGILIGVYGSSVIYMVQLPIFGVID | |
| | | TPCWIVKAAPSCSGKKGNYACLLREDQGWYCQNAG | |
| | | STVYYPNEKDCETRGDHVFCDTAAGINVAEQSKEC | |
| | | NINISTTNYPCKVSTGRHPISMVALSPLGALVACY | |
| | | KGVSCSIGSNRVGIIKQLNKGCSYITNQDADTVTI | |
| | | DNTVYQLSKVEGEQHVIKGRPVSSSFDPIKFPEDQ | |
| | | FNVALDQVFESIENSQALVDQSNRILSSAEKGNTG | |
| | | FIIVIILIAVLGSSMILVSIFIIIKKTKKPTGAPP | |
| | | ELSGVTNNGFIPHS | |
| hMPV | A63C, | MSWKVMIIISLLITPQHGLKESYLEESCSTITEGY | 180 |
| F protein | A140C, | LSVLRTGWYTNVFTLEVGDVENLTCTDCPSLIKTE | |
| | A147C, | LDLTKSALRELKTVSADQLAREEQIEGGGGGGFVL | |
| | K188C, | GAIALGVATAAAVTAGIAIAKTIRLESEVNAIKGC | |
| | K450C, | LKTTNECVSTLGNGVRVLATAVRELKEFVSKNLTS | |
| | S470C, | AINKNKCDIADLCMAVSFSQFNRRFLNVVRQFSDN | |
| | N97G, | AGITPAISLDLMTDAELARAVSYMPTSAGQIKLML | |
| | P98G, | ENRAMVRRKGFGILIGVYGSSVIYMVQLPIFGVID | |
| | R99G, | TPCWIIKAAPSCSEKDGNYACLLREDQGWYCKNAG | |
| | Q100G, | STVYYPNDKDCETRGDHVFCDTAAGINVAEQSREC | |
| | S101G, | NINISTTNYPCKVSTGRHPISMVALSPLGALVACY | |
| | R102G | KGVSCSIGSNRVGIIKQLPKGCSYITNQDADTVTI | |
| | | DNTVYQLSKVEGEQHVIKGRPVSSSFDPICFPEDQ | |
| | | FNVALDQVFESIENCQA | |
| hMPV | T127C, | MSWKVVIIFSLLITPQHGLKESYLEESCSTITEGY | 181 |
| F protein | N153C, | LSVLRTGWYTNVFTLEVGDVENLTCADGPSLIKTE | |
| | T365C, | LDLTKSALRELRTVSADQLAREEQIEGGGGGGFVL | |
| | V463C, | GAIALGVATAAAVTAGVAIAKCIRLESEVTAIKNA | |
| | A185P, | LKKTNEAVSTLGCGVRVLATAVRELKDFVSKNLTR | |
| | L219K, | AINKNKCDIPDLKMAVSFSQFNRRFLNVVRQFSDN | |
| | V231I, | AGITPAISKDLMTDAELARAISNMPTSAGQIKLML | |
| | G294E, | ENRAMVRRKGFGILIGVYGSSVIYMVQLPIFGVID | |
| | N97G, | TPCWIVKAAPSCSEKKGNYACLLREDQGWYCQNAG | |
| | P98G, | STVYYPNEKDCETRGDHVFCDTAAGINVAEQSKEC | |
| | R99G, | NINISTTNYPCKVSCGRNPISMVALSPLGALVACY | |
| | Q100G, | KGVSCSIGSNRVGIIKQLNKGCSYITNQDADTVTI | |
| | H368N, | DNTVYQLSKVEGEQHVIKGRPVSSSFDPVKFPEDQ | |
| | S101G, | FNVALDQCFESIENSQA | |
| | R102G | | |

In some embodiments, the hMPV F protein ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 179. In some embodiments, the hMPV F protein ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 180. In some embodiments, the hMPV F protein ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 181.

### C-Terminal Helix-Forming Segment

Illustrative sequences of C-terminal alpha-helical segments are shown in Table 6B (Rosetta remodel). Residues 468-470 of the native hMPV F protein are included as ENS (bold underline) and are, in these embodiments, conserved with the native sequence whereas many of the other amino acid residues are modified.

TABLE 6B
C-terminal Alpha-helical
segments for hMPV (Rosetta remodel)

| | | Re- | SEQ |
|---|---|---|---|
| | | modeled | ID |
| Name | Sequence | Length | NO: |
| C-Term 1 | ENSDRIKRAL | 7 | 182 |
| C-Term 2 | ENSSKIKKDL | 7 | 183 |
| C-Term 3 | ENSEKLTQAAS | 8 | 184 |
| C-Term 4 | ENSDRIKRALS | 8 | 185 |
| C-Term 5 | ENSERILSALS | 8 | 186 |
| C-Term 6 | ENSEKLAQAVS | 8 | 187 |
| C-Term 7 | ENSEILTQQAS | 8 | 188 |
| C-Term 8 | ENSERIERAIR | 8 | 189 |
| C-Term 9 | ENSDKIKRAIS | 8 | 190 |
| C-Term 10 | ENSERIDKAIS | 8 | 191 |
| C-Term 11 | ENSEIIKQAIS | 8 | 192 |
| C-Term 12 | ENSDRSERAQK | 8 | 193 |
| C-Term 13 | ENSTKIEKAITS | 9 | 194 |
| C-Term 14 | ENSDRIERASKS | 9 | 195 |
| C-Term 15 | ENSETIEKKLQS | 9 | 196 |
| C-Term 16 | ENSERIDEAIKR | 9 | 197 |
| C-Term 17 | ENSQKILDAIKS | 9 | 198 |
| C-Term 18 | ENSERIESAIKS | 9 | 199 |
| C-Term 19 | ENSERITKALOS | 9 | 200 |
| C-Term 20 | ENSERIEEAIRR | 9 | 201 |
| C-Term 21 | ENSEITDRKNKKA | 10 | 202 |
| C-Term 22 | ENSDRIKKALSKL | 10 | 203 |
| C-Term 23 | ENSEIAKQLMTKA | 10 | 204 |
| C-Term 24 | ENSDKIKRAITKT | 10 | 205 |
| C-Term 25 | ENSERLERHLRSR | 10 | 206 |
| C-Term 26 | ENSQKILDEIKKT | 10 | 207 |
| C-Term 27 | ENSESIKEAIKQS | 10 | 208 |
| C-Term 28 | ENSIRTKQAIKSA | 10 | 209 |
| C-Term 29 | ENSEKIKQTMKKAS | 11 | 210 |
| C-Term 30 | ENSSRIKKILSEAS | 11 | 211 |
| C-Term 31 | ENSETIKKLLKKAM | 11 | 212 |
| C-Term 32 | ENSEKIKQIARLAS | 11 | 213 |
| C-Term 33 | ENSETILTTNKRAN | 11 | 214 |
| C-Term 34 | ENSQIIQDTIKKMS | 11 | 215 |
| C-Term 35 | ENSEKILQAIRLAS | 11 | 216 |
| C-Term 36 | ENSEKIEQTRRLAS | 11 | 217 |
| C-Term 37 | ENSSRLKKAADKAS | 11 | 218 |
| C-Term 38 | ENSTKIAEAIKRTS | 11 | 219 |
| C-Term 39 | ENSERINQALKKAD | 11 | 220 |
| C-Term 40 | ENSERIKNAIKKME | 11 | 221 |
| C-Term 41 | ENSERLDKDAKTAK | 11 | 222 |
| C-Term 42 | ENSDKLKRTAEKAKS | 12 | 223 |
| C-Term 43 | ENSEEIKTLAKELKE | 12 | 224 |
| C-Term 44 | ENSESSKKAQKQAKS | 12 | 225 |
| C-Term 45 | ENSEEIKKETKRIRS | 12 | 226 |
| C-Term 46 | ENSEKMTKKANTAES | 12 | 227 |
| C-Term 47 | ENSEKMTKKANDAES | 12 | 228 |
| C-Term 48 | ENSEKIERAIKKAQS | 12 | 229 |
| C-Term 49 | ENSEYLAQVAEKVDK | 12 | 230 |
| C-Term 50 | ENSEKIERAIKKASS | 12 | 231 |
| C-Term 51 | ENSEKIERAIKYALS | 12 | 232 |
| C-Term 52 | ENSEKIERAIRKLES | 12 | 233 |
| C-Term 53 | ENSERIDSAIKKALS | 12 | 234 |
| C-Term 54 | ENSIKIKQQIKRLDEK | 13 | 235 |
| C-Term 55 | ENSEKLKRATEKARKS | 13 | 236 |
| C-Term 56 | ENSETILRAIKKAQKS | 13 | 237 |
| C-Term 57 | ENSEYLLAVAETLNRR | 13 | 238 |
| C-Term 58 | ENSEEIDTLAKELKES | 13 | 239 |
| C-Term 59 | ENSIKIKTAAKQAKKK | 13 | 240 |
| C-Term 60 | ENSERIKETNKATKQK | 13 | 241 |
| C-Term 61 | ENSAKIETAIRKTIES | 13 | 242 |
| C-Term 62 | ENSEEIKRAIEALRKR | 13 | 243 |
| C-Term 63 | ENSSRIKAMIKKILKS | 13 | 244 |
| C-Term 64 | ENSEYILTAIKIMLTR | 13 | 245 |
| C-Term 65 | ENSEKQKKINEMATKVT | 14 | 246 |
| C-Term 66 | ENSERLKKAAEIVERQT | 14 | 247 |
| C-Term 67 | ENSETIKKIIEEILSRS | 14 | 248 |
| C-Term 68 | ENSEYLKKVAEIVNKIS | 14 | 249 |
| C-Term 69 | ENSERTEKAIKITLTIS | 14 | 250 |
| C-Term 70 | ENSETLEKVAKEVTKIS | 14 | 251 |
| C-Term 71 | ENSDELKRVITDLRKLK | 14 | 252 |
| C-Term 72 | ENSTETKKAIEIALKIS | 14 | 253 |
| C-Term 73 | ENSEKITKAIEEMKKQS | 14 | 254 |
| C-Term 74 | ENSEKLEKAMEETKKLS | 14 | 255 |
| C-Term 75 | ENSEKILTAIKIALAAVS | 15 | 256 |
| C-Term 76 | ENSERLDKTAKETKEYLS | 15 | 257 |
| C-Term 77 | ENSDKIKKAVSWVLAVKS | 15 | 258 |
| C-Term 78 | ENSERIKSAIKKLESQES | 15 | 259 |
| C-Term 79 | ENSEKIKSALELALRLAK | 15 | 260 |
| C-Term 80 | ENSERIEEAIRRASKNDG | 15 | 261 |
| C-Term 81 | ENSEKLEKLERKTRQKDS | 15 | 262 |
| C-Term 82 | ENSEKIKQAIELTLKLAS | 15 | 263 |
| C-Term 83 | ENSEAIERTLKTIDKKVS | 15 | 264 |
| C-Term 84 | ENSEELKKVAKEAKKAIS | 15 | 265 |
| C-Term 85 | ENSAKIEKTLKKLKTEDS | 15 | 266 |
| C-Term 86 | ENSSKLEEALRWVTKVRS | 15 | 267 |
| C-Term 87 | ENSARIKKTIEIVLTQTS | 15 | 268 |
| C-Term 88 | ENSDRLIKVAEKTSKMLKS | 16 | 269 |
| C-Term 89 | ENSQILLDAMTNTERALRS | 16 | 270 |
| C-Term 90 | ENSDRLKKMLEKTSKMLKS | 16 | 271 |
| C-Term 91 | ENSEKIKRAIDIVEKLTOS | 16 | 272 |
| C-Term 92 | ENSESIERAIKSTKEAIKS | 16 | 273 |
| C-Term 93 | ENSERIKRALEKLTKATKS | 16 | 274 |
| C-Term 94 | ENSETIEKKLKTIESRLKS | 16 | 275 |
| C-Term 95 | ENSEKIKQAIEYMLKVAKS | 16 | 276 |
| C-Term 96 | ENSETTKKAIELLKKLYKS | 16 | 277 |
| C-Term 97 | ENSEDLKKTAAEAKKHIKS | 16 | 278 |
| C-Term 98 | ENSETIKKHIEIAIKFIKEV | 17 | 279 |
| C-Term 99 | ENSAKLTKATKYALTVIKQS | 17 | 280 |
| C-Term 100 | ENSEEIEKAIKILKKILKES | 17 | 281 |
| C-Term 101 | ENSEELKKAASKAKEEIKRS | 17 | 282 |
| C-Term 102 | ENSERIKKAIKTAIEAMQKS | 17 | 283 |
| C-Term 103 | ENSEKIEKILKELEKEKQSR | 17 | 284 |
| C-Term 104 | ENSEEIKTIISILKELEKRS | 17 | 285 |
| C-Term 105 | ENSETLKKQASKAEELEKRS | 17 | 286 |
| C-Term 106 | ENSSRLKAELKKLKEILKKS | 17 | 287 |
| C-Term 107 | ENSEYIEKAIKAAQETIKKL | 17 | 289 |
| C-Term 108 | ENSERIEKILKELEKEKQSR | 17 | 290 |
| C-Term 109 | ENSREIIRAINIVRKIASEK | 17 | 291 |
| C-Term 110 | ENSEAIERAIKDMLTAKKQS | 17 | 292 |
| C-Term 111 | ENSEEILRAIKTARTESKKT | 17 | 293 |
| C-Term 112 | ENSEKIKKAIEKAESIIQSIS | 18 | 294 |
| C-Term 113 | ENSEETKQAIKLVKKDYKEKS | 18 | 295 |
| C-Term 114 | ENSEEIDKAIKILKKILKELS | 18 | 296 |
| C-Term 115 | ENSEKTKKAIKITEEIYKKLS | 18 | 297 |
| C-Term 116 | ENSAKAEHAIKFALSEEKSRS | 18 | 298 |
| C-Term 117 | ENSERIKKAIKTANEHLSKVN | 18 | 299 |
| C-Term 118 | ENSEIIKQEIKKTQTFIKKVS | 18 | 300 |
| C-Term 119 | ENSETIKREIKKTREMTKKLL | 18 | 301 |
| C-Term 120 | ENSDKASKAIEYAERDAKSKS | 18 | 302 |
| C-Term 121 | ENSEIWETNTERSEKKVKSIQS | 19 | 303 |
| C-Term 122 | ENSEIWETNTERSIKAVLSIQS | 19 | 304 |
| C-Term 123 | ENSEKIERAIKWIEDLLKKEKS | 19 | 305 |
| C-Term 124 | ENSEEIKKAIKEARKAIEKLKS | 19 | 306 |
| C-Term 125 | ENSEEIDKAIKEARKAIEKLKS | 19 | 307 |
| C-Term 126 | ENSAKIETTKKITEELLDRAIK | 19 | 308 |
| C-Term 127 | ENSEKISQAIDKTTKIILSIES | 19 | 309 |
| C-Term 128 | ENSERIKQAIKKVEETLKRLKS | 19 | 310 |
| C-Term 129 | ENSERLEKALQTLTKAMKKTLS | 19 | 311 |
| C-Term 130 | ENSSEIKKVITETRKITKKIKSS | 20 | 312 |
| C-Term 131 | ENSAKLKETTERTEKIEKKIKDS | 20 | 313 |
| C-Term 132 | ENSDKLTRTAQKAKTLIEETKKS | 20 | 314 |
| C-Term 133 | ENSEEIKKAIKILKKILKELSSS | 20 | 315 |
| C-Term 134 | ENSDKLTRIAQKALTLIEETKKS | 20 | 316 |
| C-Term 135 | ENSIRWEANAKKAETEIKKLSES | 20 | 317 |
| C-Term 136 | ENSDELARAATLAKQLITKIKKS | 20 | 318 |
| C-Term 137 | ENSSKIETAIKKLIEKERKTRAKK | 21 | 319 |
| C-Term 138 | ENSERIKKAIEIMLSWKKALEKNS | 21 | 320 |
| C-Term 139 | ENSERIKKTAKIAQKLYKTLKSQS | 21 | 321 |
| C-Term 140 | ENSERIDKTAKIAQKLYKTLKSQS | 21 | 322 |
| C-Term 141 | ENSEKITKAIKIAKELKKLIESML | 21 | 323 |
| C-Term 142 | ENSEKITKAIKIAKELLKKIESML | 21 | 324 |
| C-Term 143 | ENSEELAQTARLAKAYLKELKSRS | 21 | 325 |
| C-Term 144 | ENSEKLKKAIEQMLTVKKITEKWS | 21 | 326 |

In some embodiments, the C-terminal helix-forming segment comprises between 5 and about 25 residues. In some embodiments, the C-terminal helix-forming segment comprises between 5 and about 20 residues. In some embodiments, the C-terminal helix-forming segment comprises between 5 and about 15 residues. In some embodiments, the C-terminal helix-forming segment comprises between 5 and about 10 residues. In some embodiments, the C-terminal helix-forming segment comprises between 10 and about 25 residues. In some embodiments, the C-terminal helix-forming segment comprises between 10 and about 20 residues. In some embodiments, the C-terminal helix-forming segment comprises between 10 and about 15 residues.

In some embodiments, modeling suggests that the following substitutions will stabilize the portion of the F protein in a helical conformation. Illustrative sequences of possible substitutions are shown in Table 6C.

TABLE 6C
Possible substitutions at Positions 471-489 (Rosetta remodel)

| Position | Preferred | Illustrative substitutions |
|---|---|---|
| Q471 | Polar | A, D, E, I, Q, R, S, T |
| A472 | Polar | A, D, E, I, K, R, S, T, Y |
| L473 | Hydrophobic | A, I, L, M, Q, S, T, W |
| V474 | Polar | A, D, E, I, K, L, N, Q, S, T |
| D475 | Polar | A, D, E, H, K, N, Q, R, S, T |
| Q476 | Hydrophobic | A, D, E, H, I, K, L, M, N, Q, T, V |
| S477 | Hydrophobic | A, E, I, K, L, M, N, Q, R, S, T, V |
| N478 | Polar | A, D, E, K, N, Q, R, S, T |
| R479 | Polar | A, D, E, F, I, K, L, M, N, Q, R, S, T, WY |
| I480 | Hydrophobic | A, I, L, M, R, S, T, V |
| L481 | Polar | D, E, I, K, L, M, N, Q, R, S, T |
| S482 | Polar | A, D, E, K, Q, R, S, T |
| S483 | Hydrophobic | A, D, E, F, H, I, K, L, M, N, Q, R, S, T, V, |
| | | W, Y |
| A484 | Hydrophobic | A, D, E, I, K, L, M, R, S, T, V, Y |
| E485 | Polar | D, E, G, K, L, Q, R, S, T |
| K486 | Polar | A, E, I, K, L, Q, R, S, T |
| G487 | Hydrophobic | A, E, I, K, L, R, S, T, V |
| N488 | Hydrophobic | E, I, K, L, N, Q, R, S |
| T489 | Polar | A, D, E, K, S |

Illustrative sequences of C-terminal alpha-helical segments are shown in Table 6D (RFdiffusion). Residues 469-471 of the native hMPV F protein are included as NSQ (bold underline) and are, in these embodiments, conserved with the native sequence whereas many of the other amino acid residues are modified.

TABLE 6D
C-terminal Alpha-helical
segments for hMPV (RFdiffusion)

| | | Re- | SEQ |
|---|---|---|---|
| | | modeled | ID |
| Name | Sequence | Length | NO: |
| C-Term 1 | NSQTTEEQIKTLTERVESIEKEG | 20 | 555 |
| C-Term 2 | NSQNIEDRVEDNDDKVAELKEELEAIK | 24 | 556 |
| C-Term 3 | NSQNVEDRLEELESRIKKIEEEIEEIK | 26 | 557 |
| | KD | | |
| C-Term 4 | NSQNIEEDLESLKERIHRLESEVQNLL | 26 | 558 |
| | ER | | |
| C-Term 5 | NSQKIQDAVEELQTLMQKL | 16 | 559 |
| C-Term 6 | NSQRTEKRINDLESRVARIEEVLSL | 22 | 560 |
| C-Term 7 | NSQETEDTLESLSQEVEKLRETVEKLT | 24 | 561 |
| C-Term 8 | NSQNILDRINENEQRVSVLERTLAQ | 22 | 562 |
| C-Term 9 | NSQSIEDSLSTLNTKINKLKKEVESLK | 30 | 563 |
| | REVEEL | | |
| C-Term 10 | NSQEIDKKLEYLEERVHDLEERLESLV | 28 | 564 |
| | QQLQ | | |
| C-Term 11 | NSQNVEDRLEANEKAISHIEQLIDQLI | 24 | 565 |

In some embodiments, the C-terminal helix-forming segment comprises between 15 and about 35 residues. In some embodiments, the C-terminal helix-forming segment comprises between 15 and about 30 residues. In some embodiments, the C-terminal helix-forming segment comprises between 15 and about 25 residues. In some embodiments, the C-terminal helix-forming segment comprises between 15 and about 20 residues. In some embodiments, the C-terminal helix-forming segment comprises between 20 and about 25 residues. In some embodiments, the C-terminal helix-forming segment comprises between 25 and about 30 residues.

In some embodiments, modeling suggests that the following substitutions will stabilize the portion of the F protein in a helical conformation. Illustrative sequences of possible substitutions are shown in Table 6E.

TABLE 6E
Possible substitutions at Positions 472-498 (RFdiffusion)

| Position | Preferred | Illustrative substitutions |
|---|---|---|
| A472 | Polar | T, N, K, R, E, S |
| L473 | Hydrophobic | T, I, V |
| V474 | Polar | E, Q, L, D |
| D475 | Polar | E, D, K |
| Q476 | Polar | Q, R, D, A, T, S, K |
| S477 | Hydrophobic | I, V, L |
| N478 | Polar | K, E, N, S |
| R479 | Polar | T, D, E, S, Y, A |
| I480 | Hydrophobic | L, N |
| L481 | Polar | T, D, E, K, Q, S, N |
| S482 | Polar | E, D, S, T, Q, K |
| S483 | Polar | R, K, L, E, A |
| A484 | Hydrophobic | V, I, M |
| E485 | Polar | E, A, K, H, Q, S, N |
| K486 | Polar | S, E, K, R, V, D, H |
| G487 | Hydrophobic | I, L |
| N488 | Polar | E, K, R |
| T489 | Polar | K, E, S, R, Q |
| S490 | Polar | E, V, T, R, L |
| G491 | Hydrophobic | GL, I, V |
| R492 | Polar | E, Q, S, A, D |
| E493 | Polar | A, E, N, L, K, Q, S |
| N494 | Hydrophobic | I, L |
| L495 | Polar | K, L, T, V, I |
| Y496 | Polar | K, E, R, Q |
| F497 | Polar | D, R, E, Q |
| Q498 | Hydrophobic | V, L |

In some embodiments, the recombinant polypeptide comprises an engineered ectodomain of an hMPV fusion (F) protein, wherein the ectodomain comprises a C-terminal helix-forming segment, between about residue 470 and about residue 500 relative to SEQ ID NO: 104, comprising one or more amino acid substitutions selected such that the segment forms a stable alpha-helical homotrimer. In some embodiments, the C-terminal helix-forming segment comprises substitutions relative to the reference sequence SEQ ID NO: 104 at two or more, three or more, or four or more residues that generate hydrophobic contacts between the segments in the alpha-helical homotrimer. In some embodiments, the ectodomain comprises the C-terminal helix-forming segment, between about residue 470 and about residue 490 relative to SEQ ID NO: 104, comprising one or more amino acid substitutions selected such that the segment forms a stable alpha-helical homotrimer. In some embodiments, the C-terminal helix-forming segment comprises between about 7 and about 21 residues.

In some embodiments, the segment comprises (1) an amino acid substitution at position Q471 relative to SEQ ID NO: 104, wherein Q is substituted with any one of A, D, E, I, Q, R, S, T, (2) an amino acid substitution at position A472 relative to SEQ ID NO: 104, wherein A is substituted with any one of A, D, E, I, K, R, S, T, Y, (3) an amino acid substitution at position L473 relative to SEQ ID NO: 104, wherein L is substituted with any one of A, I, L, M, Q, S, T, W, (4) an amino acid substitution at position V474 relative to SEQ ID NO: 104, wherein V is substituted with any one of A, D, E, I, K, L, N, Q, S, T, (5) an amino acid substitution at position D475 relative to SEQ ID NO: 104, wherein D is substituted with any one of A, D, E, H, K, N, Q, R, S, T, (6) an amino acid substitution at position Q476 relative to SEQ ID NO: 104, wherein Q is substituted with any one of A, D, E, H, I, K, L, M, N, Q, T, V, (7) an amino acid substitution at position S477 relative to SEQ ID NO: 104, wherein S is substituted with any one of A, E, I, K, L, M, N, Q, R, S, T, V, (8) an amino acid substitution at position N478 relative to SEQ ID NO: 104, wherein N is substituted with any one of A, D, E, K, N, Q, R, S, T, (9) an amino acid substitution at position R479 relative to SEQ ID NO: 104, wherein R is substituted with any one of A, D, E, F, I, K, L, M, N, Q, R, S, T, W, Y, (10) an amino acid substitution at position I480 relative to SEQ ID NO: 104, wherein I is substituted with any one of A, I, L, M, R, S, T, V, (11) an amino acid substitution at position L481 relative to SEQ ID NO: 104, wherein L is substituted with any one of D, E, I, K, L, M, N, Q, R, S, T, (12) an amino acid substitution at position S482 relative to SEQ ID NO: 104, wherein S is substituted with any one of A, D, E, K, Q, R, S, T, (13) an amino acid substitution at position S483 relative to SEQ ID NO: 104, wherein S is substituted with any one of A, D, E, F, H, I, K, L, M, N, Q, R, S, T, V, W, Y, (14) an amino acid substitution at position A484 relative to SEQ ID NO: 104, wherein A is substituted with any one of A, D, E, I, K, L, M, R, S, T, V, Y, (15) an amino acid substitution at position E485 relative to SEQ ID NO: 104, wherein E is substituted with any one of D, E, G, K, L, Q, R, S, T, (16) an amino acid substitution at position K486 relative to SEQ ID NO: 104, wherein K is substituted with any one of A, E, I, K, L, Q, R, S, T, (17) an amino acid substitution at position G487 relative to SEQ ID NO: 104, wherein G is substituted with any one of A, E, I, K, L, R, S, T, V, (18) an amino acid substitution at position N488 relative to SEQ ID NO: 104, wherein N is substituted with any one of E, I, K, L, N, Q, R, S, (19) an amino acid substitution at position T489 relative to SEQ ID NO: 104, wherein T is substituted with any one of A, D, E, K, S, and/or (20) any combination of (1)-(19). In some embodiments, the segment comprises a polypeptide sequence listed in Table 6B, or a polypeptide sequence having between 1 and 5 amino acid substitutions thereto.

In some embodiments, the ectodomain comprises the C-terminal helix-forming segment, between about residue 470 and about residue 500 relative to SEQ ID NO: 104, comprising one or more amino acid substitutions selected such that the segment forms a stable alpha-helical homotrimer. In some embodiments, the C-terminal helix-forming segment comprises between about 16 and about 30 residues.

In some embodiments, the segment comprises (1) an amino acid substitution at position A472 relative to SEQ ID NO: 104, wherein A is substituted with any one of T, N, K, R, E, S, (2) an amino acid substitution at position L473 relative to SEQ ID NO: 104, wherein L is substituted with any one of T, I, V, (3) an amino acid substitution at position V474 relative to SEQ ID NO: 104, wherein V is substituted with any one of E, Q, L, D, (4) an amino acid substitution at position D475 relative to SEQ ID NO: 104, wherein D is substituted with any one of E, D, K, (5) an amino acid substitution at position Q476 relative to SEQ ID NO: 104, wherein Q is substituted with any one of Q, R, D, A, T, S, K, (6) an amino acid substitution at position S477 relative to SEQ ID NO: 104, wherein S is substituted with any one of I, V, L, (7) an amino acid substitution at position N478 relative to SEQ ID NO: 104, wherein N is substituted with any one of K, E, N, S, (8) an amino acid substitution at position R479 relative to SEQ ID NO: 104, wherein R is substituted with any one of T, D, E, S, Y, A, (9) an amino acid substitution at position I480 relative to SEQ ID NO: 104, wherein I is substituted with any one of L, N, (10) an amino acid substitution at position L481 relative to SEQ ID NO: 104, wherein L is substituted with any one of T, D, E, K, Q, S, N, (11) an amino acid substitution at position S482 relative to SEQ ID NO: 104, wherein S is substituted with any one of E, D, S, T, Q, K, (12) an amino acid substitution at position S483 relative to SEQ ID NO: 104, wherein S is substituted with any one of R, K, L, E, A, (13) an amino acid substitution at position A484 relative to SEQ ID NO: 104, wherein A is substituted with any one of V, I, M, (14) an amino acid substitution at position E485 relative to SEQ ID NO: 104, wherein E is substituted with any one of E, A, K, H, Q, S, N, (15) an amino acid substitution at position K486 relative to SEQ ID NO: 104, wherein K is substituted with any one of S, E, K, R, V, D, H, (16) an amino acid substitution at position G487 relative to SEQ ID NO: 104, wherein G is substituted with any one of I, L, (17) an amino acid substitution at position N488 relative to SEQ ID NO: 104, wherein N is substituted with any one of E, K, R, (18) an amino acid substitution at position T489 relative to SEQ ID NO: 104, wherein T is substituted with any one of K, E, S, R, Q, (19) an amino acid substitution at position S490 relative to SEQ ID NO: 104, wherein S is substituted with any one of E, V, T, R, L, (20) an amino acid substitution at position G491 relative to SEQ ID NO: 104, wherein G is substituted with any one of GL, I, V, (21) an amino acid substitution at position R492 relative to SEQ ID NO: 104, wherein R is substituted with any one of E, Q, S, A, D, (22) an amino acid substitution at position E493 relative to SEQ ID NO: 104, wherein E is substituted with any one of A, E, N, L, K, Q, S, (23) an amino acid substitution at position N494 relative to SEQ ID NO: 104, wherein N is substituted with any one of I, L, (24) an amino acid substitution at position L495 relative to SEQ ID NO: 104, wherein L is substituted with any one of K, L, T, V, I, (25) an amino acid substitution at position Y496 relative to SEQ ID NO: 104, wherein Y is substituted with any one of K, E, R, Q, (26) an amino acid substitution at position F497 relative to SEQ ID NO: 104, wherein F is substituted with any one of D, R, E, Q, (27) an amino acid substitution at position Q498 relative to SEQ ID NO: 104, wherein Q is substituted with any one of V, L, and/or (28) any combination of (1)-(27). In some embodiments, the segment comprises a polypeptide sequence listed in Table 6D, or a polypeptide sequence having between 1 and 5 amino acid substitutions thereto.

In some embodiments, the ectodomain further comprises one, two, three or more amino acid substitutions at positions 63, 97, 98, 99, 100, 101, 102, 140, 147, 153, 185, 188, 219, 231, 294, 365, 368, 450, 463, or 470 relative to SEQ ID NO: 104.

### Human Parainfluenza Virus Type 3 (PIV3) and Type 5 (PIV5)

PIV is a negative-sense, single-stranded RNA virus which causes a variety of respiratory illnesses. It is a major cause of ubiquitous acute respiratory infections of infancy and early childhood. PIV F protein facilitates viral fusion and cell entry.

Illustrative sequences of a native PIV3 F protein are shown in Table 7A.

TABLE 7A

| | | | SEQ |
|---|---|---|---|
| | De- | | ID |
| | scription | Sequence | NO: |
| PIV3 F | Reference | MPTSILLIITTMIMASFCQIDITKLQHVG | 327 |
| protein | sequence | VLVNSPKGMKISQNFETRYLILSLIPKIE | |
| | | DSNSCGDQQIKQYKRLLDRLIIPLYDGLR | |
| | | LQKDVIVSNQESNENTDPRTKRFFGGVIG | |
| | | TIALGVATSAQITAAVALVEAKQARSDIE | |
| | | KLKEAIRDTNKAVQSVQSSIGNLIVAIKS | |
| | | VQDYVNKEIVPSIARLGCEAAGLQLGIAL | |
| | | TQHYSELTNIFGDNIGSLQEKGIKLQGIA | |
| | | SLYRTNITEIFTTSTVDKYDIYDLLFTES | |
| | | IKVRVIDVDLNDYSITLQVRLPLLTRLLN | |
| | | TQIYRVDSISYNIQNREWYIPLPSHIMTK | |
| | | GAFLGGADVKECIEAFSSYICPSDPGFVL | |
| | | NHEMESCLSGNISQCPRTVVKSDIVPRYA | |
| | | FVNGGVVANCITTTCTCNGIGNRINQPPD | |
| | | QGVKIITHKECNTIGINGMLFNTNKEGTL | |
| | | AFYTPNDITLNNSVALDPIDISIELNKAK | |
| | | SDLEESKEWIRRSNQKLDSIGNWHQSSTT | |
| | | IIIVLIMIIILFIINVTIIIIAVKYYRIQ | |
| | | KRNRVDQNDKPYVLINK | |

In some embodiments, the ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 327.

### C-Terminal Helix-Forming Segment

Illustrative sequences of C-terminal alpha-helical segments are shown in Table 7B (Rosetta remodel). Residues 456-459 of the native PIV3 F protein are included as ISIE (bold underline) and are, in these embodiments, conserved with the native sequence whereas many of the other amino acid residues are modified.

TABLE 7B
C-terminal Alpha-helical
segments for PIV3 (Rosetta remodel)

| | | Re- | SEQ |
|---|---|---|---|
| | | modeled | ID |
| Name | Sequence | Length | NO: |
| C-Term 1 | ISIELNKLAKEVKTILKELSKKLSSLES | 24 | 328 |
| C-Term 2 | ISIEMNRLKKKLDQLWKILKEDKDKS | 22 | 329 |
| C-Term 3 | ISIELNKVKSKTETMAEKMRSKETATS | 23 | 330 |
| C-Term 4 | ISIELNKVKSKTETYIKETRSKETATS | 23 | 331 |
| C-Term 5 | ISIEMNRLKSKLDKLLKELKEDKDKS | 22 | 332 |
| C-Term 6 | ISIELNKVKKETKTFIKEVRSKETATS | 23 | 333 |
| C-Term 7 | ISIEVNKTQKKLKEIWKKLKKELTKERN | 28 | 334 |
| | TLKS | | |
| C-Term 8 | ISIEVNKLKSELKTWIKQEANEKA | 20 | 335 |
| C-Term 9 | ISIELNKVKSKTETYIKEVRSKETA | 21 | 336 |
| C-Term 10 | ISIELNKLAKEVKTILKKLSKKLSSLES | 24 | 337 |

In some embodiments, the C-terminal helix-forming segment comprises between 20 and about 30 residues. In some embodiments, the C-terminal helix-forming segment comprises between 20 and about 25 residues. In some embodiments, the C-terminal helix-forming segment comprises between 25 and about 30 residues.

In some embodiments, modeling suggests that the following substitutions will stabilize the portion of the F protein in a helical conformation. Illustrative sequences of possible substitutions are shown in Table 7C.

TABLE 7C
Possible substitutions at Positions 460-477 (Rosetta remodel)

| Position | Preferred | Illustrative substitutions |
|---|---|---|
| L460 | Hydrophobic | L, M, V |
| N461 | Polar (WT) | N |
| K462 | Polar | K, R |
| V463 or | Hydrophobic | L, V, T |
| A463 | | |
| K464 | Polar | A, K, Q |
| S465 | Polar | K, S |
| D466 | Polar | E, K |
| L467 | Hydrophobic | V, L, T |
| E468 | Polar | K, D, E |
| E469 | Polar | T, Q, K, E |
| S470 | Hydrophobic | I, L, M, Y, F, W |
| K471 | Hydrophobic | L, W, A, I |
| E472 | Polar | K, E |
| W473 | Polar | E, I, K, Q |
| Y474 | Hydrophobic | L, M, T, V, E |
| R475 | Polar | S, K, R, A |
| R476 | Polar | K, E, S, N |
| S477 | Polar | K, D, E |

Illustrative sequences of C-terminal alpha-helical segments are shown in Table 7D (RFdiffusion). Residues 456-464 of the native MPV F protein are included as ISIELNKAK (bold underline) (alternatively, ISIELNKVK) and are, in these embodiments, conserved with the native sequence whereas many of the other amino acid residues are modified.

TABLE 7D
C-terminal Alpha-helical
segments for PIV3 (RFdiffusion)

| | | Remodeled | SEQ |
|---|---|---|---|
| Name | Sequence | Length | ID NO: |
| C-Term 1 | ISIELNKVKEDIEKLEERVHAIEKK | 16 | 338 |
| C-Term 2 | ISIELNKVKERVKSLEKQLKTLL | 14 | 339 |
| C-Term 3 | ISIELNKVKKKVSELEKRVDHIEHRLKQI | 20 | 340 |
| C-Term 4 | ISIELNKVKDKVEKDTKKIKEIEHELA | 18 | 341 |
| C-Term 5 | ISIELNKVKKELEELLQKVKDLEEKVETL | 20 | 342 |
| C-Term 6 | ISIELNKVKKMVESLESKVTKLEKTVKELLT | 22 | 343 |
| C-Term 7 | ISIELNKVKSELDKLKKKVEHIENS | 16 | 344 |
| C-Term 8 | ISIELNKVKKDVEKLKKRISHIEKLLS | 18 | 345 |
| C-Term 9 | ISIELNKVKKEVRKLEHEIHEIKKRLA | 18 | 346 |
| C-Term 10 | ISIELNKVKNRVEKLEETLTRLINA | 16 | 347 |
| C-Term 11 | ISIELNKVKDDLESVNKRVSEIEHELHEIKA | 22 | 348 |
| C-Term 12 | ISIELNKVKEEVKELTEEIHELREEVEALKEEL | 24 | 349 |
| C-Term 13 | ISIELNKVKQQVEKLIERLHRLENKLAEA | 20 | 350 |
| C-Term 14 | ISIELNKVKTELHKLKERVRDIEKKLA | 18 | 351 |
| C-Term 15 | ISIELNKVKKEVEELRKRLKKLEEKLTSV | 20 | 352 |
| C-Term 16 | ISIELNKVKKKVSELEKQVTEIEKILTEIRA | 22 | 353 |
| C-Term 17 | ISIELNKVKERLHKLEESVKQLKKA | 16 | 354 |
| C-Term 18 | ISIELNKVKSDVENLKEKINKII | 14 | 355 |
| C-Term 19 | ISIELNKVKDDVRTIKKELEELKQLVKNL | 20 | 356 |
| C-Term 20 | ISIELNKVKTRVEEIERKISSLEKEVEDIRRSLQQ | 26 | 357 |
| C-Term 21 | ISIELNKVKNKLEKVESQVHRLENRIEKIERLLKS | 26 | 358 |
| C-Term 22 | ISIELNKVKRDVEQLRQELNSLSKRVHKIEEAL | 24 | 359 |
| C-Term 23 | ISIELNKVKSAVTHLTKEVTKLKEL | 16 | 360 |
| C-Term 24 | ISIELNKVKKDLNDAKKRISHIEKVLN | 18 | 361 |
| C-Term 25 | ISIELNKVKADLTTLESKQSEIERRVAKIEHAL | 24 | 362 |
| C-Term 26 | ISIELNKVKEEVEKLERETKKLSHEIKKIKETL | 24 | 363 |
| C-Term 27 | ISIELNKVKSEVSELKTKVQTLETRIKKIEHELKL | 26 | 364 |
| C-Term 28 | ISIELNKVKKKVEKIEKEIEKLKRELETVKREI | 24 | 365 |
| C-Term 29 | ISIELNKVKKKVESLERKVSKLENEIKTIID | 22 | 366 |
| C-Term 30 | ISIELNKVKKDVTYLKTEVAQLQ | 14 | 367 |
| C-Term 31 | ISIELNKVKKEVKELKERLDHVEKRLKEVEEKL | 24 | 368 |
| C-Term 32 | ISIELNKVKEDVASLKKEVEKIIKA | 16 | 369 |
| C-Term 33 | ISIELNKVKNSLDKVEKKVTSLI | 14 | 370 |
| C-Term 34 | ISIELNKVKERVKENEKIITKIQKTLD | 18 | 371 |
| C-Term 35 | ISIELNKVKTEVKEITKKVRELEERLRKVEEVVKS | 26 | 372 |
| C-Term 36 | ISIELNKVKSDVRDLEERLHKLETRLEEI | 20 | 373 |
| C-Term 37 | ISIELNKVKSEVKKLKERLEELEAR | 16 | 374 |
| C-Term 38 | ISIELNKVKEKVDKIQENIDAIKTILD | 18 | 375 |
| C-Term 39 | ISIELNKVKNEVSELEKRTTKIESTIKTLIE | 22 | 376 |
| C-Term 40 | ISIELNKVKKDLKELSEKVHELLNS | 16 | 377 |
| C-Term 41 | ISIELNKVKKRLEELEEKLDRLEHIVHLL | 20 | 378 |
| C-Term 42 | ISIELNKVKENVEEIEHKVKEIE | 14 | 379 |
| C-Term 43 | ISIELNKVKKEVNELNKRIRSLEQRVEKLERALKK | 26 | 380 |
| C-Term 44 | ISIELNKVKKDLKKTKENLKEVEEKVKELLS | 22 | 381 |

In some embodiments, the C-terminal helix-forming segment comprises between 10 and about 30 residues. In some embodiments, the C-terminal helix-forming segment comprises between 10 and about 25 residues. In some embodiments, the C-terminal helix-forming segment comprises between 10 and about 20 residues. In some embodiments, the C-terminal helix-forming segment comprises between 10 and about 15 residues. In some embodiments, the C-terminal helix-forming segment comprises between 15 and about 30 residues. In some embodiments, the C-terminal helix-forming segment comprises between 15 and about 25 residues. In some embodiments, the C-terminal helix-forming segment comprises between 15 and about 20 residues. In some embodiments, the C-terminal helix-forming segment comprises between 20 and about 30 residues. In some embodiments, the C-terminal helix-forming segment comprises between 20 and about 25 residues. In some embodiments, the C-terminal helix-forming segment comprises between 25 and about 30 residues.

In some embodiments, modeling suggests that the following substitutions will stabilize the portion of the F protein in a helical conformation. Illustrative sequences of possible substitutions are shown in Table 7E.

TABLE 7E
Possible substitutions at Positions 465-486 (RF diffusion)

| Position | Preferred | Illustrative substitutions |
|---|---|---|
| S465 | Polar | E, K, D, S, N, Q, T, R, A |
| D466 | Polar | D, R, K, E, M, Q, A, S, N |
| L467 | Hydrophobic | I, V, L |
| E468 | Polar | E, K, S, D, R, H, T, N, A |
| E469 | Polar | K, S, E, N, T, Q, H, D, Y |
| S470 | Hydrophobic | L, D, V, I, A, N, T |
| K471 | Polar | E, T, L, K, N, I, R, Q, S |
| E472 | Polar | E, K, Q, S, H, R, T |
| W473 | Polar | R, Q, K, E, T, S, I, N |
| Y474 | Hydrophobic | V, L, I, Q, T |
| R475 | Polar | H, K, D, T, E, S, R, N, Q, A |
| R476 | Polar | A, T, H, E, D, K, R, Q, S |
| S477 | Hydrophobic | I, L, V |
| N478 | Polar | E, L, K, I, R, S, S |
| Q479 | Polar | K, H, E, N, Q, R, T, A, S |
| K480 | Polar | K, R, E, T, S, L, A, I, V |
| L481 | Hydrophobic | L, V, I |
| D482 | Polar | K, A, E, S, H, T, N, D, R |
| S483 | Polar | Q, T, E, A, S, N, D, K, L |
| I484 | Hydrophobic | I, L, A, V |
| G485 | Polar | L, K, R, E, I |
| S486 | Polar | T, A, E, R, H, D, S |

In some embodiments, the recombinant polypeptide comprises an engineered ectodomain of a PIV3 fusion (F) protein, wherein the ectodomain comprises a C-terminal helix-forming segment, between about residue 460 and about residue 490 relative to SEQ ID NO: 327, comprising one or more amino acid substitutions selected such that the segment forms a stable alpha-helical homotrimer. In some embodiments, the C-terminal helix-forming segment comprises substitutions relative to the reference sequence SEQ ID NO: 327 at two or more, three or more, or four or more residues that generate hydrophobic contacts between the segments in the alpha-helical homotrimer. In some embodiments, the ectodomain comprises the C-terminal helix-forming segment, between about residue 460 and about residue 480 relative to SEQ ID NO: 104, comprising one or more amino acid substitutions selected such that the segment forms a stable alpha-helical homotrimer. In some embodiments, the C-terminal helix-forming segment comprises between about 20 and about 28 residues.

In some embodiments, the segment comprises (1) an amino acid substitution at position L460 relative to SEQ ID NO: 327, wherein L is substituted with any one of L, M, V, (2) an amino acid substitution at position N461 relative to SEQ ID NO: 327, wherein N is substituted with N, (3) an amino acid substitution at position K462 relative to SEQ ID NO: 327, wherein K is substituted with any one of K, R, (4) an amino acid substitution at position V463 relative to SEQ ID NO: 327, wherein V is substituted with any one of L, V, T, (5) an amino acid substitution at position K464 relative to SEQ ID NO: 327, wherein K is substituted with any one of A, K, Q, (6) an amino acid substitution at position S465 relative to SEQ ID NO: 327, wherein S is substituted with any one of K, S, (7) an amino acid substitution at position D466 relative to SEQ ID NO: 327, wherein D is substituted with any one of E, K, (8) an amino acid substitution at position L467 relative to SEQ ID NO: 327, wherein L is substituted with any one of V, L, T, (9) an amino acid substitution at position E468 relative to SEQ ID NO: 327, wherein E is substituted with any one of K, D, E, (10) an amino acid substitution at position E469 relative to SEQ ID NO: 327, wherein E is substituted with any one of T, Q, K, E, (11) an amino acid substitution at position S470 relative to SEQ ID NO: 327, wherein S is substituted with any one of I, L, M, Y, F, W, (12) an amino acid substitution at position K471 relative to SEQ ID NO: 327, wherein K is substituted with any one of L, W, A, I, (13) an amino acid substitution at position E472 relative to SEQ ID NO: 327, wherein E is substituted with any one of K, E, (14) an amino acid substitution at position W473 relative to SEQ ID NO: 327, wherein W is substituted with any one of E, I, K, Q, (15) an amino acid substitution at position Y474 relative to SEQ ID NO: 327, wherein Y is substituted with any one of L, M, T, V, E, (16) an amino acid substitution at position R475 relative to SEQ ID NO: 327, wherein R is substituted with any one of S, K, R, A, (17) an amino acid substitution at position R476 relative to SEQ ID NO: 327, wherein R is substituted with any one of K, E, S, N, (18) an amino acid substitution at position S477 relative to SEQ ID NO: 327, wherein S is substituted with any one of K, D, E, and/or (19) any combination of (1)-(18).

In some embodiments, the segment comprises a polypeptide sequence listed in Table 7B, or a polypeptide sequence having between 1 and 5 amino acid substitutions thereto. In some embodiments, the ectodomain comprises the C-terminal helix-forming segment, between about residue 465 and about residue 490 relative to SEQ ID NO: 327, comprising one or more amino acid substitutions selected such that the segment forms a stable alpha-helical homotrimer. In some embodiments, the C-terminal helix-forming segment comprises between about 14 and about 30 residues.

In some embodiments, the segment comprises (1) an amino acid substitution at position S465 relative to SEQ ID NO: 327, wherein S is substituted with any one of E, K, D, S, N, Q, T, R, A, (2) an amino acid substitution at position D466 relative to SEQ ID NO: 327, wherein D is substituted with any one of D, R, K, E, M, Q, A, S, N, (3) an amino acid substitution at position L467 relative to SEQ ID NO: 327, wherein L is substituted with any one of I, V, L, (4) an amino acid substitution at position E468 relative to SEQ ID NO: 327, wherein E is substituted with any one of E, K, S, D, R, H, T, N, A, (5) an amino acid substitution at position E469 relative to SEQ ID NO: 327, wherein E is substituted with any one of K, S, E, N, T, Q, H, D, Y, (6) an amino acid substitution at position S470 relative to SEQ ID NO: 327, wherein S is substituted with any one of L, D, V, I, A, N, T, (7) an amino acid substitution at position K471 relative to SEQ ID NO: 327, wherein K is substituted with any one of E, T, L, K, N, I, R, Q, S, (8) an amino acid substitution at position E472 relative to SEQ ID NO: 327, wherein E is substituted with any one of E, K, Q, S, H, R, T, (9) an amino acid substitution at position W473 relative to SEQ ID NO: 327, wherein W is substituted with any one of R, Q, K, E, T, S, I, N, (10) an amino acid substitution at position Y474 relative to SEQ ID NO: 327, wherein Y is substituted with any one of V, L, I, Q, T, (11) an amino acid substitution at position R475 relative to SEQ ID NO: 327, wherein R is substituted with any one of H, K, D, T, E, S, R, N, Q, A, (12) an amino acid substitution at position R476 relative to SEQ ID NO: 327, wherein R is substituted with any one of A, T, H, E, D, K, R, Q, S, (13) an amino acid substitution at position S477 relative to SEQ ID NO: 327, wherein S is substituted with any one of I, L, V, (14) an amino acid substitution at position N478 relative to SEQ ID NO: 327, wherein N is substituted with any one of E, L, K, I, R, S, Q, (15) an amino acid substitution at position Q479 relative to SEQ ID NO: 327, wherein Q is substituted with any one of K, H, E, N, Q, R, T, A, S, (16) an amino acid substitution at position K480 relative to SEQ ID NO: 327, wherein K is substituted with any one of K, R, E, T, S, L, A, I, V, (17) an amino acid substitution at position L481 relative to SEQ ID NO: 327, wherein L is substituted with any one of L, V, I, (18) an amino acid substitution at position D482 relative to SEQ ID NO: 327, wherein D is substituted with any one of K, A, E, S, H, T, N, D, R, (19) an amino acid substitution at position S483 relative to SEQ ID NO: 327, wherein S is substituted with any one of Q, T, E, A, S, N, D, K, L, (20) an amino acid substitution at position I484 relative to SEQ ID NO: 327, wherein I is substituted with any one of I, L, A, V, (21) an amino acid substitution at position G485 relative to SEQ ID NO: 327, wherein G is substituted with any one of L, K, R, E, I, (22) an amino acid substitution at position S486 relative to SEQ ID NO: 327, wherein S is substituted with any one of T, A, E, R, H, D, S, and/or (23) any combination of (1)-(22). In some embodiments, the segment comprises a polypeptide sequence listed in Table 7D, or a polypeptide sequence having between 1 and 5 amino acid substitutions thereto.

Illustrative sequences of a native PIV5 F protein are shown in Table 8A.

TABLE 8A

| | | | SEQ |
|---|---|---|---|
| | De- | | ID |
| | scription | Sequence | NO: |
| PIV5 F | Reference | MGTIIQFLVVSCLLAGAGSLDPAALMQIG | 382 |
| protein | sequence | VIPTNVRQLMYYTEASSAFIVVKLMPTID | |
| | | SPISGCNITSISSYNATVTKLLQPIGENL | |
| | | ETIRNQLIPTRRRRRFAGVVIGLAALGVA | |
| | | TAAQVTAAVALVKANENAAAILNLKNAIQ | |
| | | KTNAAVADVVQATQSLGTAVQAVQDHINS | |
| | | VVSPAITAANCKAQDAIIGSILNLYLTEL | |
| | | TTIFHNQITNPALSPITIQALRILLGSTL | |
| | | PTVVEKSFNTQISAAELLSSGLLTGQIVG | |
| | | LDLTYMQMVIKIELPTLTVQPATQIIDLA | |
| | | TISAFINNQEVMAQLPTRVMVTGSLIQAY | |
| | | PASQCTITPNTVYCRYNDAQVLSDDTMAC | |
| | | LQGNLTRCTFSPVVGSFLTREVLFDGIVY | |
| | | ANCRSMLCKCMQPAAVILQPSSSPVTVID | |
| | | MYKCVSLQLDNLRFTITQLANVTYNSTIK | |
| | | LESSQILSIDPLDISQNLAAVNKSLSDAL | |
| | | QHLAQSDTYLSAITSATTTSVLSIIAICL | |
| | | GSLGLILIILLSVVVWKLLTIVVANRNRM | |
| | | ENFVYHK | |

In some embodiments, the PIV5 protein ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 382.

### C-Terminal Helix-Forming Segment

Illustrative sequences of C-terminal alpha-helical segments are shown in Table 8B (Rosetta remodel). Residues 459-462 of the native PIV5 F protein are included as SLSD (bold underline) and are, in these embodiments, conserved with the native sequence whereas many of the other amino acid residues are modified.

TABLE 8B
C-terminal Alpha-helical
segments for PIV5 (Rosetta remodel)

| | | Re- | SEQ |
|---|---|---|---|
| | | modeled | ID |
| Name | Sequence | Length | NO: |
| C-Term 1 | SLSDLKKKVDEATKTT | 12 | 383 |
| C-Term 2 | SLSDLIKAITKKEEKSTRKERSERKS | 22 | 384 |
| C-Term 3 | SLSDTIKKLDKLVKS | 11 | 385 |
| C-Term 4 | SLSDLIKEVKS | 7 | 386 |
| C-Term 5 | SLSDTQKLVTEILEKLTK | 14 | 387 |
| C-Term 6 | SLSDVIQIMLETLETATKQKKKDS | 20 | 388 |
| C-Term 7 | SLSDLAKKFKEAS | 9 | 389 |
| C-Term 8 | SLSDLKKKLDELEKR | 11 | 390 |
| C-Term 9 | SLSDTIKKVDKSTKSTEKKS | 16 | 391 |
| C-Term 10 | SLSDVAKKLEEKIRTDIKREQS | 18 | 392 |
| C-Term 11 | SLSDTITIMKKIEEKLKADKKKSS | 20 | 393 |
| C-Term 12 | SLSDVIKWVREVVSKWIS | 14 | 394 |
| C-Term 13 | SLSDLKKKVDTLEKQS | 12 | 395 |
| C-Term 14 | SLSDLWKIMEKLS | 9 | 396 |
| C-Term 15 | SLSDLKKKVDSK | 8 | 397 |
| C-Term 16 | SLSDLAKKLDKTIEKASKDDSKKS | 20 | 398 |
| C-Term 17 | SLSDVAKRAESTIRDLKETKK | 17 | 399 |
| C-Term 18 | SLSDLATKVEKALS | 10 | 400 |
| C-Term 19 | SLSDLIKKTDALEKS | 11 | 401 |
| C-Term 20 | SLSDLIKKVITLEKKS | 12 | 402 |
| C-Term 21 | SLSDLKKKTEEIATDLEKKWRKMSKS | 22 | 403 |
| C-Term 22 | SLSDLKKKLDSILTEQKRRS | 16 | 404 |
| C-Term 23 | SLSDVIKKLDEALSRI | 12 | 405 |
| C-Term 24 | SLSDTIKEMKEK | 8 | 406 |
| C-Term 25 | SLSDLAEKCKKLKKKLEEDLKS | 18 | 407 |
| C-Term 26 | SLSDVIKEIRKLKS | 10 | 408 |
| C-Term 27 | SLSDLAKIVKSLIS | 10 | 409 |
| C-Term 28 | SLSDLKKKLEEILASIEKKEKS | 18 | 410 |
| C-Term 29 | SLSDTIKELKSHLTTLKIEKSKKS | 20 | 411 |
| C-Term 30 | SLSDLKEKLDRYI | 9 | 412 |
| C-Term 31 | SLSDLKTKIEQILKS | 11 | 413 |
| C-Term 32 | SLSDVIKKLDKIVKKLQS | 14 | 414 |
| C-Term 33 | SLSDLASKVETETRK | 11 | 415 |
| C-Term 34 | SLSDLAKRTKTWYDILAKILASNQKS | 22 | 416 |
| C-Term 35 | SLSDTAKIALTVEKILTTRDK | 17 | 417 |
| C-Term 36 | SLSDTQKLLKELI | 9 | 418 |
| C-Term 37 | SLSDVIKKVETIASKLKS | 14 | 419 |
| C-Term 38 | SLSDAIKKIDKLES | 10 | 420 |
| C-Term 39 | SLSDTISILEEFLRRYKQKE | 16 | 421 |
| C-Term 40 | SLSDTQKQLETLAKKIKS | 14 | 422 |
| C-Term 41 | SLSDLAKRVKKYWEEVKSRS | 16 | 423 |
| C-Term 42 | SLSDLAKELKKLKEHILRYQ | 16 | 424 |
| C-Term 43 | SLSDTIKLVIKAILTAIKEK | 16 | 425 |
| C-Term 44 | SLSDTIKKVDKLTS | 10 | 426 |
| C-Term 45 | SLSDTIKKLEKLERELRSRWDSERKS | 22 | 427 |
| C-Term 46 | SLSDTIKTTEKALKIILKRIKKALAE | 26 | 428 |
| | QKSS | | |
| C-Term 47 | SLSDLIKKFNS | 7 | 429 |
| C-Term 48 | SLSDLKKTLEKR | 8 | 430 |
| C-Term 49 | SLSDLESELKSRLS | 10 | 431 |
| C-Term 50 | SLSDVIKDLKKTK | 9 | 432 |
| C-Term 51 | SLSDLAKKLDS | 7 | 433 |
| C-Term 52 | SLSDVIKIIESQTRS | 11 | 434 |
| C-Term 53 | SLSDLKKETEKLKKKV | 12 | 435 |
| C-Term 54 | SLSDAIKRVLSWYKKKADEESS | 18 | 436 |
| C-Term 55 | SLSDVKKKVDKAITEIKS | 14 | 437 |
| C-Term 56 | SLSDLAKEVKKK | 8 | 438 |
| C-Term 57 | SLSDLKKKLEKIL | 9 | 439 |
| C-Term 58 | SLSDLASDVSSMKAT | 11 | 440 |
| C-Term 59 | SLSDTIKKLEELTTK | 11 | 441 |
| C-Term 60 | SLSDLKKTTEKVIRTLKTKE | 16 | 442 |
| C-Term 61 | SLSDLKKEHEELLKEIKKQK | 16 | 443 |
| C-Term 62 | SLSDLATKTKQLEEKLEKEK | 16 | 444 |
| C-Term 63 | SLSDLKKRTIKWYEETLKRT | 16 | 445 |
| C-Term 64 | SLSDLAKKTKEAIDRIRS | 14 | 446 |
| C-Term 65 | SLSDLQTDIKRLKS | 10 | 447 |
| C-Term 66 | SLSDLAKKTKELEKKIKS | 14 | 448 |
| C-Term 67 | SLSDLAKKAKKFTEKLLSEIKKTKSD | 22 | 449 |
| C-Term 68 | SLSDLAKYVS | 6 | 450 |
| C-Term 69 | SLSDTQKKTKETATKLEQKTEKTLKY | 26 | 451 |
| | TKKK | | |
| C-Term 70 | SLSDLKKKVDKK | 8 | 452 |
| C-Term 71 | SLSDLARKTKEYWEKEERSKKS | 18 | 453 |
| C-Term 72 | SLSDLKKRLEDYIKTQKAKS | 16 | 454 |
| C-Term 73 | SLSDLKKKLDELTKKS | 12 | 455 |
| C-Term 74 | SLSDLIKEVK | 6 | 456 |
| C-Term 75 | SLSDVIKILKEIKEMLDKLLEKSKKS | 22 | 457 |
| C-Term 76 | SLSDLAKQTKKLEDELRS | 14 | 458 |

In some embodiments, the C-terminal helix-forming segment comprises between 5 and about 30 residues. In some embodiments, the C-terminal helix-forming segment comprises between 5 and about 25 residues. In some embodiments, the C-terminal helix-forming segment comprises between 5 and about 20 residues. In some embodiments, the C-terminal helix-forming segment comprises between 5 and about 15 residues. In some embodiments, the C-terminal helix-forming segment comprises between 5 and about 10 residues. In some embodiments, the C-terminal helix-forming segment comprises between 10 and about 30 residues. In some embodiments, the C-terminal helix-forming segment comprises between 10 and about 25 residues. In some embodiments, the C-terminal helix-forming segment comprises between 10 and about 20 residues. In some embodiments, the C-terminal helix-forming segment comprises between 10 and about 15 residues. In some embodiments, the C-terminal helix-forming segment comprises between 15 and about 30 residues. In some embodiments, the C-terminal helix-forming segment comprises between 15 and about 25 residues. In some embodiments, the C-terminal helix-forming segment comprises between 15 and about 20 residues.

In some embodiments, modeling suggests that the following substitutions will stabilize the portion of the F protein in a helical conformation. Illustrative sequences of possible substitutions are shown in Table 8C.

TABLE 8C
Possible substitutions at Positions 463-488 (Rosetta remodel)

| Position | Preferred | Illustrative substitutions |
|---|---|---|
| A463 | Hydrophobic | L, T, V, A |
| L464 | Polar | K, I, Q, A, W, E |
| Q465 | Polar | K, Q, T, E, S, R |
| H466 | Polar | K, A, E, L, I, W, R, Q, T, D, Y |
| L467 | Hydrophobic | V, I, L, M, FA, T, C, H |
| A468 | Polar | D, T, K, L, E, R, I, N, S |
| Q469 | Polar | E, K, S, T, A, R, Q, D |
| S470 | Hydrophobic | A, K, L, I, T, S, V, H, Y, E, W, FR, Q, M |
| D471 | Hydrophobic | T, E, V, L, S, I, A, K, Y, W |
| T472 | Polar | K, E, R, S, T, A, D, L |
| Y473 | Polar | T, K, S, R, Q, D, E, I, H, M |
| L474 | Hydrophobic | T, S, L, A, D, W, Q, I, Y, V, K, E |
| S475 | Polar | T, E, I, K, S, Q, A, L, R, D |
| A476 | Polar | R, K, A, S, E, I, T, D, Q |
| I477 | Polar | K, Q, R, D, T, E, I, Y, S, L |
| T478 | Hydrophobic | E, K, S, D, W, L, Q, I, T |
| S479 | Polar | R, K, Q, S, A, D, E |
| A480 | Polar | S, K |
| T481 | Hydrophobic | E, D, S, K, M, N, A, T |
| T482 | Hydrophobic | R, S, Q, L, K |
| T483 | Polar | K, A, S |
| S484 | Polar | S, E, D, Y |
| V485 | Polar | Q, T |
| L486 | Polar | K |
| S487 | Polar | S, K |
| I488 | Polar | S, K |

In some embodiments, the recombinant polypeptide comprises an engineered ectodomain of a PIV5 fusion (F) protein, wherein the ectodomain comprises a C-terminal helix-forming segment, between about residue 460 and about residue 490 relative to SEQ ID NO: 382, comprising one or more amino acid substitutions selected such that the segment forms a stable alpha-helical homotrimer. In some embodiments, the C-terminal helix-forming segment comprises substitutions relative to the reference sequence SEQ ID NO: 382 at two or more, three or more, or four or more residues that generate hydrophobic contacts between the segments in the alpha-helical homotrimer. In some embodiments, the ectodomain comprises the C-terminal helix-forming segment, between about residue 460 and about residue 480 relative to SEQ ID NO: 104, comprising one or more amino acid substitutions selected such that the segment forms a stable alpha-helical homotrimer. In some embodiments, the C-terminal helix-forming segment comprises between about 6 and about 26 residues.

In some embodiments, the segment comprises (1) an amino acid substitution at position A463 relative to SEQ ID NO: 382, wherein A is substituted with any one of L, T, V, A, (2) an amino acid substitution at position L464 relative to SEQ ID NO: 382, wherein L is substituted with any one of K, I, Q, A, W, E, (3) an amino acid substitution at position Q465 relative to SEQ ID NO: 382, wherein Q is substituted with any one of K, Q, T, E, S, R, (4) an amino acid substitution at position H466 relative to SEQ ID NO: 382, wherein H is substituted with any one of K, A, E, L, I, W, R, Q, T, D, Y, (5) an amino acid substitution at position L467 relative to SEQ ID NO: 382, wherein Lis substituted with any one of V, I, L, M, FA, T, C, H, (6) an amino acid substitution at position A468 relative to SEQ ID NO: 382, wherein A is substituted with any one of D, T, K, L, E, R, I, N, S, (7) an amino acid substitution at position Q469 relative to SEQ ID NO: 382, wherein Q is substituted with any one of E, K, S, T, A, R, Q, D, (8) an amino acid substitution at position S470 relative to SEQ ID NO: 382, wherein S is substituted with any one of A, K, L, I, T, S, V, H, Y, E, W, FR, Q, M, (9) an amino acid substitution at position D471 relative to SEQ ID NO: 382, wherein D is substituted with any one of T, E, V, L, S, I, A, K, Y, W, (10) an amino acid substitution at position T472 relative to SEQ ID NO: 382, wherein T is substituted with any one of K, E, R, S, T, A, D, L, (11) an amino acid substitution at position Y473 relative to SEQ ID NO: 382, wherein Y is substituted with any one of T, K, S, R, Q, D, E, I, H, M, (12) an amino acid substitution at position L474 relative to SEQ ID NO: 382, wherein L is substituted with any one of T, S, L, A, D, W, Q, I, Y, V, K, E, (13) an amino acid substitution at position S475 relative to SEQ ID NO: 382, wherein S is substituted with any one of T, E, I, K, S, Q, A, L, R, D, (14) an amino acid substitution at position A476 relative to SEQ ID NO: 382, wherein A is substituted with any one of R, K, A, S, E, I, T, D, Q, (15) an amino acid substitution at position I477 relative to SEQ ID NO: 382, wherein I is substituted with any one of K, Q, R, D, T, E, I, Y, S, L, (16) an amino acid substitution at position T478 relative to SEQ ID NO: 382, wherein Tis substituted with any one of E, K, S, D, W, L, Q, I, T, (17) an amino acid substitution at position S479 relative to SEQ ID NO: 382, wherein S is substituted with any one of R, K, Q, S, A, D, E, (18) an amino acid substitution at position A480 relative to SEQ ID NO: 382, wherein A is substituted with any one of S, K, (19) an amino acid substitution at position T481 relative to SEQ ID NO: 382, wherein T is substituted with any one of E, D, S, K, M, N, A, T, (20) an amino acid substitution at position T482 relative to SEQ ID NO: 382, wherein T is substituted with any one of R, S, Q, L, K, (21) an amino acid substitution at position T483 relative to SEQ ID NO: 382, wherein Tis substituted with any one of K, A, S, (22) an amino acid substitution at position S484 relative to SEQ ID NO: 382, wherein S is substituted with any one of S, E, D, Y, (23) an amino acid substitution at position V485 relative to SEQ ID NO: 382, wherein V is substituted with any one of Q, T, (24) an amino acid substitution at position L486 relative to SEQ ID NO: 382, wherein L is substituted with K, (25) an amino acid substitution at position S487 relative to SEQ ID NO: 382, wherein S is substituted with any one of S, K, (26) an amino acid substitution at position I488 relative to SEQ ID NO: 382, wherein I is substituted with any one of S, K, and/or (27) any combination of (1)-(26).

In some embodiments, the segment comprises a polypeptide sequence listed in Table 8B, or a polypeptide sequence having between 1 and 5 amino acid substitutions thereto.

### SARS-COV-2

SARS-COV-2 is a single, positive-strand RNA virus which can cause severe respiratory disease in humans. The SARS COV-2 viral spike(S) protein, which is a homotrimeric class I fusion glycoprotein, binds to angiotensin-converting enzyme 2 (ACE2), which is the entry receptor utilized by SARS-COV-2. The spike(S) protein of coronaviruses is a major surface protein and is a target for neutralizing antibodies in infected subjects or patients. Therefore, it is considered a potential protective antigen for vaccine design.

TABLE 9A

| | De- | | SEQ |
|---|---|---|---|
| | scrip- | | ID |
| | tion | Sequence | NO: |
| SARS- | Refer- | MFVFLVLLPLVSSQCVNLTTRTQLPPAYTNSFTRG | 459 |
| CoV-2 | ence | VYYPDKVFRSSVLHSTQDLFLPFFSNVTWFHAIHV | |
| Spike | se- | SGTNGTKRFDNPVLPFNDGVYFASTEKSNIIRGWI | |
| pro- | quence | FGTTLDSKTQSLLIVNNATNVVIKVCEFQFCNDPF | |
| tein | | LGVYYHKNNKSWMESEFRVYSSANNCTFEYVSQPF | |
| | | LMDLEGKQGNFKNLREFVFKNIDGYFKIYSKHTPI | |
| | | NLVRDLPQGFSALEPLVDLPIGINITRFQTLLALH | |
| | | RSYLTPGDSSSGWTAGAAAYYVGYLQPRTFLLKYN | |
| | | ENGTITDAVDCALDPLSETKCTLKSFTVEKGIYQT | |
| | | SNFRVQPTESIVRFPNITNLCPFGEVFNATRFASV | |
| | | YAWNRKRISNCVADYSVLYNSASFSTFKCYGVSPT | |
| | | KLNDLCFTNVYADSFVIRGDEVRQIAPGQTGKIAD | |
| | | YNYKLPDDFTGCVIAWNSNNLDSKVGGNYNYLYRL | |
| | | FRKSNLKPFERDISTEIYQAGSTPCNGVEGFNCYF | |
| | | PLQSYGFQPTNGVGYQPYRVVVLSFELLHAPATVC | |
| | | GPKKSTNLVKNKCVNFNFNGLTGTGVLTESNKKFL | |
| | | PFQQFGRDIADTTDAVRDPQTLEILDITPCSFGGV | |
| | | SVITPGTNTSNQVAVLYQDVNCTEVPVAIHADQLT | |
| | | PTWRVYSTGSNVFQTRAGCLIGAEHVNNSYECDIP | |
| | | IGAGICASYQTQTNSPRRARSVASQSIIAYTMSLG | |
| | | AENSVAYSNNSIAIPTNFTISVTTEILPVSMTKTS | |
| | | VDCTMYICGDSTECSNLLLQYGSFCTQLNRALTGI | |
| | | AVEQDKNTQEVFAQVKQIYKTPPIKDFGGFNFSQI | |
| | | LPDPSKPSKRSFIEDLLFNKVTLADAGFIKQYGDC | |
| | | LGDIAARDLICAQKFNGLTVLPPLLTDEMIAQYTS | |
| | | ALLAGTITSGWTFGAGAALQIPFAMQMAYRFNGIG | |
| | | VTQNVLYENQKLIANQFNSAIGKIQDSLSSTASAL | |
| | | GKLQDVVNQNAQALNTLVKQLSSNFGAISSVLNDI | |
| | | LSRLDKVEAEVQIDRLITGRLQSLQTYVTQQLIRA | |
| | | AEIRASANLAATKMSECVLGQSKRVDFCGKGYHLM | |
| | | SFPQSAPHGVVFLHVTYVPAQEKNFTTAPAICHDG | |
| | | KAHFPREGVFVSNGTHWFVTQRNFYEPQIITTDNT | |
| | | FVSGNCDVVIGIVNNTVYDPLQPELDSFKEELDKY | |
| | | FKNHTSPDVDLGDISGINASVVNIQKEIDRLNEVA | |
| | | KNLNESLIDLQELGKYEQYIKWPWYIWLGFIAGLI | |
| | | AIVMVTIMLCCMTSCCSCLKGCCSCGSCCKFDEDD | |
| | | SEPVLKGVKLHYT | |

In some embodiments, the SARS-COV-2 spike(S) protein ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 459.

### C-Terminal Helix-Forming Segment

Illustrative sequences of C-terminal alpha-helical segments are shown in Table 9B (Rosetta remodel). Residues 1147-1170 of the native SARS-COV-2 S protein are included as LQPEL (bold underline) and are, in these embodiments, conserved with the native sequence whereas many of the other amino acid residues are modified.

TABLE 9B
C-terminal Alpha-helical segments for SARS
(Rosetta remodel)

| | | Re- | SEQ |
|---|---|---|---|
| | | modeled | ID |
| Name | Sequence | Length | NO: |
| C-Term | LQPELETAIKITLEIVLKILKEWEKRKSS | 24 | 460 |
| 1 | | | |
| C-Term | LQPELDSAASYAIKV | 10 | 461 |
| 2 | | | |
| C-Term | LQPELETAASIAEKIARKLLKES | 18 | 462 |
| 3 | | | |
| C-Term | LQPELESAIKKTLKIISKRNKDS | 18 | 463 |
| 4 | | | |
| C-Term | LQPELEKAIKKATEIARKLIS | 16 | 464 |
| 5 | | | |
| C-Term | LQPELESAADKTMKKYKTEAKRS | 18 | 465 |
| 6 | | | |
| C-Term | LQPELETALRIAIEITLQLLKKMAS | 20 | 466 |
| 7 | | | |
| C-Term | LQPELEKAIKITLKIIDIKLS | 16 | 467 |
| 8 | | | |
| C-Term | LQPELEKAAKKALEIASRS | 14 | 468 |
| 9 | | | |
| C-Term | LQPELEKAIKKTLKIIWTELSIS | 18 | 469 |
| 10 | | | |
| C-Term | LQPELESAMKTAMKIIS | 12 | 470 |
| 11 | | | |
| C-Term | LQPELKKAMETAIKRINKA | 14 | 471 |
| 12 | | | |
| C-Term | LQPELEKAAKKTLKIAKEESTKDKS | 20 | 472 |
| 13 | | | |
| C-Term | LQPELEKAIKKTLKIIRTELSIS | 18 | 473 |
| 14 | | | |
| C-Term | LQPELESAIKKALTIIKQIWS | 16 | 474 |
| 15 | | | |
| C-Term | LQPELDSAASRALKIAIELLRATESKK | 22 | 475 |
| 16 | | | |
| C-Term | LQPELEKAASKAIKISLKILKEILS | 20 | 476 |
| 17 | | | |
| C-Term | LQPELEKAIKEALKR | 10 | 477 |
| 18 | | | |
| C-Term | LQPELETAIKIALEIARKEIS | 16 | 478 |
| 19 | | | |
| C-Term | LQPELEKAAKTALKIAS | 12 | 479 |
| 20 | | | |
| C-Term | LQPELEKAAEEAVRRAIKLYKENLKKS | 22 | 480 |
| 21 | | | |

In some embodiments, the C-terminal helix-forming segment comprises between 10 and about 25 residues. In some embodiments, the C-terminal helix-forming segment comprises between 10 and about 20 residues. In some embodiments, the C-terminal helix-forming segment comprises between 10 and about 15 residues. In some embodiments, the C-terminal helix-forming segment comprises between 15 and about 25 residues. In some embodiments, the C-terminal helix-forming segment comprises between 15 and about 20 residues.

In some embodiments, modeling suggests that the following substitutions will stabilize the portion of the F protein in a helical conformation. Illustrative sequences of possible substitutions are shown in Table 9C. Numbering in this table reflects a single amino acid substitution relative to the reference sequence above.

TABLE 9C
Possible substitutions at Positions 1147-1170 (Rosetta remodel)

| Position | Preferred | Illustrative substitutions |
|---|---|---|
| D1147 | Polar | E, D, K |
| S1148 | Polar | T, S, K |
| F1149 | Alanine | A |
| K1150 | Hydrophobic | I, A, L, M |
| E1151 | Polar | K, S, D, R, E |
| E1152 | Polar | I, Y, K, T, R, E |
| L1153 | Hydrophobic | T, A |
| D1154 | Hydrophobic | L, I, E, T, M, V |
| K1155 | Polar | E, K, T, R |
| Y1156 | Hydrophobic | I, V, K, R |
| F1157 | Hydrophobic | V, A, I, Y, T, S |
| K1158 | Hydrophobic | L, R, S, K, D, W, N, I |
| N1159 | Polar | K, T, Q, I, R, E |
| H1160 | Polar | I, L, R, E, K, S |
| T1161 | Hydrophobic | L, N, I, A, S, W, Y |
| S1162 | Polar | K, S, T, R |
| P1163 | Polar | E, D, R, K, I, A |
| D1164 | Hydrophobic | W, S, M, D, T, I, N |
| V1165 | Polar | E, A, K, L |
| D1166 | Polar | K, S |
| L1167 | Polar | R, K |
| G1168 | Polar | K, S |
| D1169 | Polar | S |
| I1170 | Polar | S |

Illustrative sequences of C-terminal alpha-helical segments are shown in Table 9D (RFdiffusion). Residues 1147-1165 of the native SARS-COV-2 Spike(S) protein are included as LQPEL (bold underline) and are, in these embodiments, conserved with the native sequence whereas many of the other amino acid residues are modified.

TABLE 9D
C-terminal Alpha-helical segments for SARS
(RFdiffusion)

| | | Re- | SEQ |
|---|---|---|---|
| | | modeled | ID |
| Name | Sequence | Length | NO: |
| C-Term 1 | LQPELQTLKEESTHLTKTLLS | 16 | 481 |
| C-Term 2 | LQPELTKLKEEVLEEVETMIRETAA | 20 | 482 |
| C-Term 3 | LQPELENLKNIVESIIN | 12 | 483 |
| C-Term 4 | LQPELSKTKAETLETVREL | 14 | 484 |
| C-Term 5 | LQPELEKTQSTTLTAAKTLIKST | 18 | 485 |
| C-Term 6 | LQPELETTKKETLTEVTEA | 14 | 486 |
| C-Term 7 | LQPELERIRTEVTQASA | 12 | 487 |
| C-Term 8 | LQPELESTKAVTETEIKAEIN | 16 | 488 |
| C-Term 9 | LQPELNTTKTETISSIKKEIETM | 18 | 489 |
| C-Term | LQPELEATHTRTLTTVTAA | 14 | 490 |
| 10 | | | |
| C-Term | LQPELDTTKKETLTEAQETLERA | 18 | 491 |
| 11 | | | |
| C-Term | LQPELDKVKDETVTIMTKYIQET | 18 | 492 |
| 12 | | | |
| C-Term | LQPELDATSSRAIERVTTLLE | 16 | 493 |
| 13 | | | |
| C-Term | LQPELETTRTKTITEVNTTISTT | 18 | 494 |
| 14 | | | |
| C-Term | LQPELEAVKTETLTAATTAINSALAKQ | 22 | 495 |
| 15 | | | |
| C-Term | LQPELKETQEKTITEVIKILN | 16 | 496 |
| 16 | | | |
| C-Term | LQPELTNTENNVLTRVKQS | 14 | 497 |
| 17 | | | |
| C-Term | LQPELNALETRVLTAIN | 12 | 498 |
| 18 | | | |

In some embodiments, the C-terminal helix-forming segment comprises between 10 and about 25 residues. In some embodiments, the C-terminal helix-forming segment comprises between 10 and about 20 residues. In some embodiments, the C-terminal helix-forming segment comprises between 15 and about 25 residues.

In some embodiments, modeling suggests that the following substitutions will stabilize the portion of the F protein in a helical conformation. Illustrative sequences of possible substitutions are shown in Table 9E.

TABLE 9E
Possible substitutions at Positions 1147-1165 (RFdiffusion)

| Position | Preferred | Illustrative substitutions |
|---|---|---|
| D1147 | Polar | Q, T, E, S, N, D, K |
| S1148 | Polar | T, K, N, R, S, A, E |
| F1149 | Hydrophobic | L, T, I, V |
| K1150 | Polar | K, Q, R, H, S, E |
| E1151 | Polar | E, N, A, S, K, T, D |
| E1152 | Polar | E, T, V, R, K, N |
| L1153 | Hydrophobic | S, V, T, A |
| D1154 | Hydrophobic | T, L, E, I, V |
| K1155 | Polar | H, E, S, T, Q |
| Y1156 | Polar | L, E, I, T, A, S, R |
| F1157 | Hydrophobic | T, V, I, A, S, M |
| K1158 | Polar | K, E, N, R, T, A, Q, I |
| N1159 | Polar | T, E, A, K, Q |
| H1160 | Hydrophobic | L, M, A, E, T, Y, I, S |
| T1161 | Hydrophobic | L, I |
| S1162 | Polar | S, R, K, N, E, Q |
| P1163 | Polar | E, S, T, R |
| D1164 | Hydrophobic | T, M, A |
| V1165 | Hydrophobic | A, L |

In some embodiments, an engineered ectodomain of a SARS-COV2 spike(S) protein, wherein the ectodomain comprises a C-terminal helix-forming segment, between about residue 1140 and about residue 1170 relative to SEQ ID NO: 459, comprises one or more amino acid substitutions selected such that the segment forms a stable alpha-helical homotrimer. In some embodiments, the C-terminal helix-forming segment comprises substitutions relative to the reference sequence SEQ ID NO: 459 at two or more, three or more, or four or more residues that generate hydrophobic contacts between the segments in the alpha-helical homotrimer. In some embodiments, the ectodomain comprises the C-terminal helix-forming segment, between about residue 1140 and about residue 1170 relative to SEQ ID NO: 459, comprising one or more amino acid substitutions selected such that the segment forms a stable alpha-helical homotrimer. In some embodiments, the C-terminal helix-forming segment comprises between about 10 and about 25 residues.

In some embodiments, the segment comprises (1) an amino acid substitution at position D1147 relative to SEQ ID NO: 459, wherein D is substituted with any one of E, D, K, (2) an amino acid substitution at position S1148 relative to SEQ ID NO: 459, wherein S is substituted with any one of T, S, K, (3) an amino acid substitution at position F1149 relative to SEQ ID NO: 459, wherein F is substituted with A, (4) an amino acid substitution at position K1150 relative to SEQ ID NO: 459, wherein K is substituted with any one of I, A, L, M, (5) an amino acid substitution at position E1151 relative to SEQ ID NO: 459, wherein E is substituted with any one of K, S, D, R, E, (6) an amino acid substitution at position E1152 relative to SEQ ID NO: 459, wherein E is substituted with any one of I, Y, K, T, R, E, (7) an amino acid substitution at position L1153 relative to SEQ ID NO: 459, wherein L is substituted with any one of T, A, (8) an amino acid substitution at position D1154 relative to SEQ ID NO: 459, wherein D is substituted with any one of L, I, E, T, M, V, (9) an amino acid substitution at position K1155 relative to SEQ ID NO: 459, wherein K is substituted with any one of E, K, T, R, (10) an amino acid substitution at position Y1156 relative to SEQ ID NO: 459, wherein Y is substituted with any one of I, V, K, R, (11) an amino acid substitution at position F1157 relative to SEQ ID NO: 459, wherein F is substituted with any one of V, A, I, Y, T, S, (12) an amino acid substitution at position K1158 relative to SEQ ID NO: 459, wherein K is substituted with any one of L, R, S, K, D, W, N, I, (13) an amino acid substitution at position N1159 relative to SEQ ID NO: 459, wherein N is substituted with any one of K, T, Q, I, R, E, (14) an amino acid substitution at position H1160 relative to SEQ ID NO: 459, wherein His substituted with any one of I, L, R, E, K, S, (15) an amino acid substitution at position T1161 relative to SEQ ID NO: 459, wherein T is substituted with any one of L, N, I, A, S, W, Y, (16) an amino acid substitution at position S1162 relative to SEQ ID NO: 459, wherein S is substituted with any one of K, S, T, R, (17) an amino acid substitution at position P1163 relative to SEQ ID NO: 459, wherein P is substituted with any one of E, D, R, K, I, A, (18) an amino acid substitution at position D1164 relative to SEQ ID NO: 459, wherein D is substituted with any one of W, S, M, D, T, I, N, (19) an amino acid substitution at position V1165 relative to SEQ ID NO: 459, wherein V is substituted with any one of E, A, K, L, (20) an amino acid substitution at position D1166 relative to SEQ ID NO: 459, wherein D is substituted with any one of K, S, (21) an amino acid substitution at position L1167 relative to SEQ ID NO: 459, wherein L is substituted with any one of R, K, (22) an amino acid substitution at position G1168 relative to SEQ ID NO: 459, wherein G is substituted with any one of K, S, (23) an amino acid substitution at position D1169 relative to SEQ ID NO: 459, wherein D is substituted with S, (24) an amino acid substitution at position I1170 relative to SEQ ID NO: 459, wherein I is substituted with S, and/or (25) any combination of (1)-(24).

In some embodiments, the segment comprises a polypeptide sequence listed in Table 9B, or a polypeptide sequence having between 1 and 5 amino acid substitutions thereto. In some embodiments, the ectodomain comprises the C-terminal helix-forming segment, between about residue 1145 and about residue 1175 relative to SEQ ID NO: 459, comprising one or more amino acid substitutions selected such that the segment forms a stable alpha-helical homotrimer. In some embodiments, the C-terminal helix-forming segment comprises between about 12 and about 22 residues.

In some embodiments, the segment comprises (1) an amino acid substitution at position D1147 relative to SEQ ID NO: 459, wherein D is substituted with any one of Q, T, E, S, N, D, K, (2) an amino acid substitution at position S1148 relative to SEQ ID NO: 459, wherein S is substituted with any one of T, K, N, R, S, A, E, (3) an amino acid substitution at position F1149 relative to SEQ ID NO: 459, wherein F is substituted with any one of L, T, I, V, (4) an amino acid substitution at position K1150 relative to SEQ ID NO: 459, wherein K is substituted with any one of K, Q, R, H, S, E, (5) an amino acid substitution at position E1151 relative to SEQ ID NO: 459, wherein E is substituted with any one of E, N, A, S, K, T, D, (6) an amino acid substitution at position E1152 relative to SEQ ID NO: 459, wherein E is substituted with any one of E, T, V, R, K, N, (7) an amino acid substitution at position L1153 relative to SEQ ID NO: 459, wherein L is substituted with any one of S, V, T, A, (8) an amino acid substitution at position D1154 relative to SEQ ID NO: 459, wherein D is substituted with any one of T, L, E, I, V, (9) an amino acid substitution at position K1155 relative to SEQ ID NO: 459, wherein K is substituted with any one of H, E, S, T, Q, (10) an amino acid substitution at position Y1156 relative to SEQ ID NO: 459, wherein Y is substituted with any one of L, E, I, T, A, S, R, (11) an amino acid substitution at position F1157 relative to SEQ ID NO: 459, wherein F is substituted with any one of T, V, I, A, S, M, (12) an amino acid substitution at position K1158 relative to SEQ ID NO: 459, wherein K is substituted with any one of K, E, N, R, T, A, Q, I, (13) an amino acid substitution at position N1159 relative to SEQ ID NO: 459, wherein N is substituted with any one of T, E, A, K, Q, (14) an amino acid substitution at position H1160 relative to SEQ ID NO: 459, wherein His substituted with any one of L, M, A, E, T, Y, I, S, (15) an amino acid substitution at position T1161 relative to SEQ ID NO: 459, wherein T is substituted with any one of L, I, (16) an amino acid substitution at position S1162 relative to SEQ ID NO: 459, wherein S is substituted with any one of S, R, K, N, E, Q, (17) an amino acid substitution at position P1163 relative to SEQ ID NO: 459, wherein P is substituted with any one of E, S, T, R, (18) an amino acid substitution at position D1164 relative to SEQ ID NO: 459, wherein D is substituted with any one of T, M, A, (19) an amino acid substitution at position V1165 relative to SEQ ID NO: 459, wherein V is substituted with any one of A, L, and/or (20) any combination of (1)-(19).

In some embodiments, the segment comprises a polypeptide sequence listed in Table 9D, or a polypeptide sequence having between 1 and 5 amino acid substitutions thereto.

### Nipah Virus

Nipah virus is a highly pathogenic virus, which has caused sporadic outbreaks of severe neurological and respiratory disease.

TABLE 10A

| | De- | | SEQ |
|---|---|---|---|
| | scrip- | | ID |
| | tion | Sequence | NO: |
| Nipah | Ref- | MVVILDKRCYCNLLILILMISECSVGILH | 499 |
| F | erence | YEKLSKIGLVKGVTRKYKIKSNPLTKDIV | |
| protein | se- | IKMIPNVSNMSQCTGSVMENYKTRLNGIL | |
| | quence | TPIKGALEIYKNNTHDLVGDVRLAGVIMA | |
| | | GVAIGIATAAQITAGVALYEAMKNADNIN | |
| | | KLKSSIESTNEAVVKLQETAEKTVYVLTA | |
| | | LQDYINTNLVPTIDKISCKQTELSLDLAL | |
| | | SKYLSDLLFVFGPNLQDPVSNSMTIQAIS | |
| | | QAFGGNYETLLRTLGYATEDFDDLLESDS | |
| | | ITGQIIYVDLSSYYIIVRVYFPILTEIQQ | |
| | | AYIQELLPVSFNNDNSEWISIVPNFILVR | |
| | | NTLISNIEIGFCLITKRSVICNQDYATPM | |
| | | TNNMRECLTGSTEKCPRELVVSSHVPRFA | |
| | | LSNGVLFANCISVTCQCQTTGRAISQSGE | |
| | | QTLLMIDNTTCPTAVLGNVIISLGKYLGS | |
| | | VNYNSEGIAIGPPVFTDKVDISSQISSMN | |
| | | QSLQQSKDYIKEAQRLLDTVNPSLISMLS | |
| | | MIILYVLSIASLCIGLITFISFIIVEKKR | |
| | | NTYSRLEDRRVRPTSSGDLYYIGT | |

In some embodiments, the Nipah F protein ectodomain is at least 70%, at least 80%, at least 90%, at least 91%, at least 92%, at least 93%, at least 94%, at least 95%, at least 96%, at least 97%, at least 98%, at least 99%, or 100% identical to SEQ ID NO: 499.

### C-Terminal Helix-Forming Segment

Illustrative sequences of C-terminal alpha-helical segments are shown in Table 10B (Rosetta remodel). Residues 460-462 of the native Nipah F protein are included as ISS (bold underline) and are, in these embodiments, conserved with the native sequence whereas many of the other amino acid residues are modified.

TABLE 10B
C-terminal Alpha-helical segments for Nipah
(Rosetta remodel)

| | | Re- | SEQ |
|---|---|---|---|
| | | modeled | ID |
| Name | Sequence | Length | NO: |
| C-Term | ISSINEDMERTKKWITKLIAKWKS | 21 | 500 |
| 1 | | | |
| C-Term | ISSINEALKSLATDVKKLKSKI | 19 | 501 |
| 2 | | | |
| C-Term | ISSANLEIEKTKRKMTSIAKEVKT | 31 | 502 |
| 3 | RIAKEEKSKS | | |
| C-Term | ISSTNLTVEKIWRYLMAVLS | 17 | 503 |
| 4 | | | |
| C-Term | ISSTNKRTATIEKIVRSLLKEIKS | 25 | 504 |
| 5 | ERTR | | |
| C-Term | ISSINETVTRLKKIVEKLIRELQK | 23 | 505 |
| 6 | IK | | |
| C-Term | ISSTNTIVSKTLKMLLEFITREER | 24 | 506 |
| 7 | SKR | | |
| C-Term | ISSTNSLTEKILQWIKKFETKVKS | 21 | 507 |
| 8 | | | |
| C-Term | ISSTNLIVTETIKELKSTDKKLKK | 29 | 508 |
| 9 | YIKTVQSS | | |
| C-Term | ISSANKIMAEIIKTIKSLLKKS | 19 | 509 |
| 10 | | | |
| C-Term | ISSANLEIEKTKRIMTSIALYVWT | 31 | 510 |
| 11 | LIAKELKSKS | | |
| C-Term | ISSINEEIKKVKKTAAEAITTQTR | 33 | 511 |
| 12 | IWQKLKKSKSKS | | |
| C-Term | ISSLNEKIDKLEKKMSTIAKKLSK | 31 | 512 |
| 13 | IEASKRKSSS | | |
| C-Term | ISSTNIRVTKTEKKVEDLLKKLTS | 21 | 513 |
| 14 | | | |
| C-Term | ISSINELVTRLAKILKKLI | 16 | 514 |
| 15 | | | |
| C-Term | ISSINEQVKKIEEILRSMS | 16 | 515 |
| 16 | | | |
| C-Term | ISSANLKIETLARIVSTWYKQQAK | 31 | 516 |
| 17 | KTATEEKRKS | | |
| C-Term | ISSMNTRIDQIEKWLRDKEKKEQS | 21 | 517 |
| 18 | | | |
| C-Term | ISSINEETKKVKKIALDIAS | 17 | 518 |
| 19 | | | |
| C-Term | ISSINEKIDSLKKEVKKYIEKAEK | 25 | 519 |
| 20 | DKKS | | |
| C-Term | ISSLNDLVRKALKWIKEVKKKS | 19 | 520 |
| 21 | | | |
| C-Term | ISSLNEKIIKILQKLLTWITKTKQ | 25 | 521 |
| 22 | EKKS | | |

In some embodiments, the C-terminal helix-forming segment comprises between 15 and about 35 residues. In some embodiments, the C-terminal helix-forming segment comprises between 15 and about 30 residues. In some embodiments, the C-terminal helix-forming segment comprises between 15 and about 25 residues. In some embodiments, the C-terminal helix-forming segment comprises between 15 and about 20 residues. In some embodiments, the C-terminal helix-forming segment comprises between 20 and about 35 residues. In some embodiments, the C-terminal helix-forming segment comprises between 20 and about 30 residues. In some embodiments, the C-terminal helix-forming segment comprises between 20 and about 25 residues. In some embodiments, the C-terminal helix-forming segment comprises between 25 and about 35 residues. In some embodiments, the C-terminal helix-forming segment comprises between 25 and about 30 residues.

In some embodiments, modeling suggests that the following substitutions will stabilize the portion of the F protein in a helical conformation. Illustrative sequences of possible substitutions are shown in Table 10C.

TABLE 10C
Possible substitutions at Positions 463-489 (Rosetta remodel)

| Position | Preferred | Illustrative substitutions |
|---|---|---|
| M463 | Hydrophobic | I, A, T, L, M |
| N464 | N | N |
| Q465 | Polar | E, L, K, T, S, I, D |
| S466 | S | S |
| L467 | Hydrophobic | M, L, I, V, T |
| Q468 | Polar | E, K, A, T, S, D, R, I, Q |
| Q469 | Polar | R, S, K, T, E, Q |
| S470 | Hydrophobic | T, L, I, V, A |
| K471 | Hydrophobic | K, A, W, E, L, I |
| D472 | Polar | K, T, R, Q, E |
| Y473 | Hydrophobic | W, D, K, Y, I, M, E, T |
| I474 | Hydrophobic | I, V, M, L, A |
| K475 | Polar | T, K, M, R, E, L, A, S |
| E476 | Polar | K, S, A, E, T, D |
| A477 | Hydrophobic | L, I, V, FT, A, M, W, K, Y |
| Q478 | Polar | I, K, A, L, E, D, S, Y |
| R479 | Polar | A, S, K, R, T, L, E |
| L480 | Polar | K, E, R, Y, T, Q |
| L481 | Hydrophobic | W, I, V, L, E, S, Q, A, T |
| D482 | Polar | K, Q, E, W, T, S, A |
| T483 | Polar | S, T, K, R, Q |
| V484 | Hydrophobic | R, E, I, S, Y, L, K, D |
| N485 | Hydrophobic | I, R, K, W, E, T |
| P486 | Polar | A, T, R, K, Q |
| S487 | Polar | K, R, T, S |
| L488 | Hydrophobic | E, V, L, K |
| I489 | Polar | E, Q, L, K, R |

Illustrative sequences of C-terminal alpha-helical segments are shown in Table 10D (RFdiffusion). Residues 460-462 of the native Nipah F protein are included as ISS (bold underline) and are, in these embodiments, conserved with the native sequence whereas many of the other amino acid residues are modified.

TABLE 10D
C-terminal Alpha-helical segments for Nipah
(RFdiffusion)

| | | Re- | SEQ |
|---|---|---|---|
| | | modeled | ID |
| Name | Sequence | Length | NO: |
| C-Term | ISSLRQKISSLEKALKKAEKDLEEVRR | 26 | 522 |
| 1 | QL | | |
| C-Term | ISSLTTEVKQLQTSL | 12 | 523 |
| 2 | | | |
| C-Term | ISSLTNSITSLSERIHKLENL | 18 | 524 |
| 3 | | | |
| C-Term | ISSLTDRLDNLEERVKRLEEEVKKLKE | 24 | 525 |
| 4 | | | |
| C-Term | ISSITEQLKEAQERVDKIEKLLEKILR | 24 | 526 |
| 5 | | | |
| C-Term | ISSLTSAITAIQETL | 12 | 527 |
| 6 | | | |
| C-Term | ISSLRKEIKELRTVVKRLL | 16 | 528 |
| 7 | | | |
| C-Term | ISSLTRSIKDVKQAL | 12 | 529 |
| 8 | | | |
| C-Term | ISSITSEITELKKTL | 12 | 530 |
| 9 | | | |
| C-Term | ISSLQKNVESLAKEVKKLEQKLNSL | 22 | 531 |
| 10 | | | |
| C-Term | ISSLRQEIKNLQDEVTKVTEELKKLVE | 26 | 532 |
| 11 | QL | | |
| C-Term | ISSVKTNVRKLSEILAS | 14 | 533 |
| 12 | | | |
| C-Term | ISSLNKKIEEIEKRLSELESTIKKL | 22 | 534 |
| 13 | | | |
| C-Term | ISSLQSLAESLADKVTALETRIKSIEA | 24 | 535 |
| 14 | | | |
| C-Term | ISSLSKRVKSVETRLRT | 14 | 536 |
| 15 | | | |
| C-Term | ISSITTDIKQNTERIDKIEKTLK | 20 | 537 |
| 16 | | | |
| C-Term | ISSLTRAVRKLEKRLTHVEEVLK | 20 | 538 |
| 17 | | | |
| C-Term | ISSITKEIKSLDTRL | 12 | 539 |
| 18 | | | |
| C-Term | ISSITKKVDSLLTEVHAIRHEIDQLRS | 24 | 540 |
| 19 | | | |
| C-Term | ISSIREQISTITTEIKKIKEILL | 20 | 541 |
| 20 | | | |
| C-Term | ISSLTDEISKLSNRVQRLERRLQEIER | 26 | 542 |
| 21 | RL | | |
| C-Term | ISSLTERVERLETLVREVQKQLE | 20 | 543 |
| 22 | | | |
| C-Term | ISSLTEKIESIEKDIAT | 14 | 544 |
| 23 | | | |
| C-Term | ISSLAKRLDELSSQLADLSARVEALQS | 26 | 545 |
| 24 | TL | | |
| C-Term | ISSLTNHIKDLAKRVSDIESLVQKLLS | 24 | 546 |
| 25 | | | |
| C-Term | ISSITSSISRNTDKIKELQQEIEKLQS | 26 | 547 |
| 26 | SL | | |
| C-Term | ISSLTRDVDKLNSQIQALI | 16 | 548 |
| 27 | | | |
| C-Term | ISSLTAVASENTARIEALERRIHELEL | 24 | 549 |
| 28 | | | |
| C-Term | ISSLKEEVTNLKKRLSEVEKVIKTL | 22 | 550 |
| 29 | | | |
| C-Term | ISSITEQLQRLSERVEEIERR | 18 | 551 |
| 30 | | | |
| C-Term | ISSLNTQVKKLKDRIKKIEERLN | 20 | 552 |
| 31 | | | |
| C-Term | ISSLQSEVSNLRTDLNDLKKLVKKLIE | 26 | 553 |
| 32 | LL | | |
| C-Term | ISSITKDIQKNTERINKIEKTIKSLIS | 24 | 554 |
| 33 | | | |

In some embodiments, the C-terminal helix-forming segment comprises between 10 and about 30 residues. In some embodiments, the C-terminal helix-forming segment comprises between 10 and about 25 residues. In some embodiments, the C-terminal helix-forming segment comprises between 10 and about 20 residues. In some embodiments, the C-terminal helix-forming segment comprises between 10 and about 15 residues. In some embodiments, the C-terminal helix-forming segment comprises between 15 and about 30 residues. In some embodiments, the C-terminal helix-forming segment comprises between 15 and about 25 residues. In some embodiments, the C-terminal helix-forming segment comprises between 15 and about 20 residues. In some embodiments, the C-terminal helix-forming segment comprises between 20 and about 30 residues. In some embodiments, the C-terminal helix-forming segment comprises between 20 and about 25 residues.

In some embodiments, modeling suggests that the following substitutions will stabilize the portion of the F protein in a helical conformation. Illustrative sequences of possible substitutions are shown in Table 10E.

TABLE 10E
Possible substitutions at Positions 463-489 (RF diffusion)

| Position | Preferred | Illustrative substitutions |
|---|---|---|
| M463 | Hydrophobic | L, I, V |
| N464 | Polar | N |
| Q465 | Polar | Q, T, N, D, E, S, K, R, A |
| S466 | Polar | S |
| L467 | Hydrophobic | I, V, L, A |
| Q468 | Polar | S, K, T, D, E, R, Q |
| Q469 | Polar | S, Q, N, E, A, D, K, T, R, |
| S470 | Hydrophobic | L, A, I, V, N, |
| K471 | Polar | E, Q, S, R, K, A, T, D, L, N |
| D472 | Polar | K, T, E, Q, D, N, S, A |
| Y473 | Polar | A, S, R, T, V, E, I, K, L, D, Q |
| I474 | Hydrophobic | L, I, V |
| K475 | Polar | K, H, D, T, A, S, R, Q, E, N, |
| E476 | Polar | K, R, S, E, A, T, H, D |
| A477 | Hydrophobic | A, L, I, V |
| Q478 | Polar | E, L, T, R, K, Q, S, I |
| R479 | Polar | K, N, E, Q, S, T, H, R, A |
| L480 | Polar | D, L, E, K, T, R, V, I, Q |
| L481 | Hydrophobic | L, V, I |
| D482 | Polar | E, K, N, D, L, Q, H |
| T483 | Polar | E, K, S, Q, A, T |
| V484 | Hydrophobic | V, L, I |
| N485 | Polar | R, K, L, V, E, Q, I |
| P486 | Polar | R, E, A, S, L |
| S487 | Polar | Q, R, T, S, L |
| L488 | Hydrophobic | L |

In some embodiments, the recombinant polypeptide comprises an engineered ectodomain of a Nipah fusion (F) protein, wherein the ectodomain comprises a C-terminal helix-forming segment, between about residue 460 and about residue 490 relative to SEQ ID NO: 499, comprising one or more amino acid substitutions selected such that the segment forms a stable alpha-helical homotrimer. In some embodiments, the C-terminal helix-forming segment comprises substitutions relative to the reference sequence SEQ ID NO: 499 at two or more, three or more, or four or more residues that generate hydrophobic contacts between the segments in the alpha-helical homotrimer. In some embodiments, the ectodomain comprises the C-terminal helix-forming segment, between about residue 460 and about residue 490 relative to SEQ ID NO: 499, comprising one or more amino acid substitutions selected such that the segment forms a stable alpha-helical homotrimer. In some embodiments, the C-terminal helix-forming segment comprises between about 16 and about 33 residues.

In some embodiments, the segment comprises (1) an amino acid substitution at position M463 relative to SEQ ID NO: 499, wherein M is substituted with any one of I, A, T, L, M, (2) an amino acid substitution at position N464 relative to SEQ ID NO: 499, wherein N is substituted with N, (3) an amino acid substitution at position Q465 relative to SEQ ID NO: 499, wherein Q is substituted with any one of E, L, K, T, S, I, D, (4) an amino acid substitution at position S466 relative to SEQ ID NO: 499, wherein S is substituted with S, (5) an amino acid substitution at position L467 relative to SEQ ID NO: 499, wherein L is substituted with any one of M, L, I, V, T, (6) an amino acid substitution at position Q468 relative to SEQ ID NO: 499, wherein Q is substituted with any one of E, K, A, T, S, D, R, I, Q, (7) an amino acid substitution at position Q469 relative to SEQ ID NO: 499, wherein Q is substituted with any one of R, S, K, T, E, Q, (8) an amino acid substitution at position S470 relative to SEQ ID NO: 499, wherein S is substituted with any one of T, L, I, V, A, (9) an amino acid substitution at position K471 relative to SEQ ID NO: 499, wherein K is substituted with any one of K, A, W, E, L, I, (10) an amino acid substitution at position D472 relative to SEQ ID NO: 499, wherein D is substituted with any one of K, T, R, Q, E, (11) an amino acid substitution at position Y473 relative to SEQ ID NO: 499, wherein Y is substituted with any one of W, D, K, Y, I, M, E, T, (12) an amino acid substitution at position I474 relative to SEQ ID NO: 499, wherein I is substituted with any one of I, V, M, L, A, (13) an amino acid substitution at position K475 relative to SEQ ID NO: 499, wherein K is substituted with any one of T, K, M, R, E, L, A, S, (14) an amino acid substitution at position E476 relative to SEQ ID NO: 499, wherein E is substituted with any one of K, S, A, E, T, D, (15) an amino acid substitution at position A477 relative to SEQ ID NO: 499, wherein A is substituted with any one of L, I, V, FT, A, M, W, K, Y, (16) an amino acid substitution at position Q478 relative to SEQ ID NO: 499, wherein Q is substituted with any one of I, K, A, L, E, D, S, Y, (17) an amino acid substitution at position R479 relative to SEQ ID NO: 499, wherein R is substituted with any one of A, S, K, R, T, L, E, (18) an amino acid substitution at position L480 relative to SEQ ID NO: 499, wherein L is substituted with any one of K, E, R, Y, T, Q, (19) an amino acid substitution at position L481 relative to SEQ ID NO: 499, wherein L is substituted with any one of W, I, V, L, E, S, Q, A, T, (20) an amino acid substitution at position D482 relative to SEQ ID NO: 499, wherein D is substituted with any one of K, Q, E, W, T, S, A, (21) an amino acid substitution at position T483 relative to SEQ ID NO: 499, wherein T is substituted with any one of S, T, K, R, Q, (22) an amino acid substitution at position V484 relative to SEQ ID NO: 499, wherein V is substituted with any one of R, E, I, S, Y, L, K, D, (23) an amino acid substitution at position N485 relative to SEQ ID NO: 499, wherein Nis substituted with any one of I, R, K, W, E, T, (24) an amino acid substitution at position P486 relative to SEQ ID NO: 499, wherein P is substituted with any one of A, T, R, K, Q, (25) an amino acid substitution at position S487 relative to SEQ ID NO: 499, wherein S is substituted with any one of K, R, T, S, (26) an amino acid substitution at position L488 relative to SEQ ID NO: 499, wherein L is substituted with any one of E, V, L, K, (27) an amino acid substitution at position I489 relative to SEQ ID NO: 499, wherein I is substituted with any one of E, Q, L, K, R, and/or (28) any combination of (1)-(27).

In some embodiments, the segment comprises a polypeptide sequence listed in Table 10B, or a polypeptide sequence having between 1 and 5 amino acid substitutions thereto. In some embodiments, the ectodomain comprises the C-terminal helix-forming segment, between about residue 460 and about residue 490 relative to SEQ ID NO: 499, comprising one or more amino acid substitutions selected such that the segment forms a stable alpha-helical homotrimer. In some embodiments, the C-terminal helix-forming segment comprises between about 12 and about 26 residues.

In some embodiments, the segment comprises (1) an amino acid substitution at position M463 relative to SEQ ID NO: 499, wherein M is substituted with any one of L, I, V, (2) an amino acid substitution at position N464 relative to SEQ ID NO: 499, wherein N is substituted with N, (3) an amino acid substitution at position Q465 relative to SEQ ID NO: 499, wherein Q is substituted with any one of Q, T, N, D, E, S, K, R, A, (4) an amino acid substitution at position S466 relative to SEQ ID NO: 499, wherein S is substituted with S, (5) an amino acid substitution at position L467 relative to SEQ ID NO: 499, wherein L is substituted with any one of I, V, L, A, (6) an amino acid substitution at position Q468 relative to SEQ ID NO: 499, wherein Q is substituted with any one of S, K, T, D, E, R, Q, (7) an amino acid substitution at position Q469 relative to SEQ ID NO: 499, wherein Q is substituted with any one of S, Q, N, E, A, D, K, T, R, (8) an amino acid substitution at position S470 relative to SEQ ID NO: 499, wherein S is substituted with any one of L, A, I, V, N, (9) an amino acid substitution at position K471 relative to SEQ ID NO: 499, wherein K is substituted with any one of E, Q, S, R, K, A, T, D, L, N, (10) an amino acid substitution at position D472 relative to SEQ ID NO: 499, wherein D is substituted with any one of K, T, E, Q, D, N, S, A, (11) an amino acid substitution at position Y473 relative to SEQ ID NO: 499, wherein Y is substituted with any one of A, S, R, T, V, E, I, K, L, D, Q, (12) an amino acid substitution at position I474 relative to SEQ ID NO: 499, wherein I is substituted with any one of L, I, V, (13) an amino acid substitution at position K475 relative to SEQ ID NO: 499, wherein K is substituted with any one of K, H, D, T, A, S, R, Q, E, N, (14) an amino acid substitution at position E476 relative to SEQ ID NO: 499, wherein E is substituted with any one of K, R, S, E, A, T, H, D, (15) an amino acid substitution at position A477 relative to SEQ ID NO: 499, wherein A is substituted with any one of A, L, I, V, (16) an amino acid substitution at position Q478 relative to SEQ ID NO: 499, wherein Q is substituted with any one of E, L, T, R, K, Q, S, I, (17) an amino acid substitution at position R479 relative to SEQ ID NO: 499, wherein R is substituted with any one of K, N, E, Q, S, T, H, R, A, (18) an amino acid substitution at position L480 relative to SEQ ID NO: 499, wherein L is substituted with any one of D, L, E, K, T, R, V, I, Q, (19) an amino acid substitution at position L481 relative to SEQ ID NO: 499, wherein L is substituted with any one of L, V, I, (20) an amino acid substitution at position D482 relative to SEQ ID NO: 499, wherein D is substituted with any one of E, K, N, D, L, Q, H, (21) an amino acid substitution at position T483 relative to SEQ ID NO: 499, wherein T is substituted with any one of E, K, S, Q, A, T, (22) an amino acid substitution at position V484 relative to SEQ ID NO: 499, wherein V is substituted with any one of V, L, I, (23) an amino acid substitution at position N485 relative to SEQ ID NO: 499, wherein N is substituted with any one of R, K, L, V, E, Q, I, (24) an amino acid substitution at position P486 relative to SEQ ID NO: 499, wherein P is substituted with any one of R, E, A, S, L, (25) an amino acid substitution at position S487 relative to SEQ ID NO: 499, wherein S is substituted with any one of Q, R, T, S, L, (26) an amino acid substitution at position L488 relative to SEQ ID NO: 499, wherein L is substituted with any one of L, and/or (27) any combination of (1)-(26).

In some embodiments, the segment comprises a polypeptide sequence listed in Table 10D, or a polypeptide sequence having between 1 and 5 amino acid substitutions thereto.

### III. Protein Nanostructures

The disclosure further provides protein nanostructures comprising any of the engineered ectodomains described herein. For example, the disclosure provides protein nanostructures comprising a trimeric component comprising a recombinant polypeptide comprising an ectodomain of a viral membrane fusion (F) protein of Respiratory Syncytial Virus (RSV) having an engineered C-terminal alpha-helical segment that stabilizes the F protein in a prefusion conformation and pentameric component.

Further provided are compositions in which any of the alpha-helical segments described herein are used as a fusion to a trimeric protein complex or to a trimeric component of a nanostructure to stabilize the complex or component. For example, the alpha-helical segments described herein may be used without any antigen (e.g., ectodomain) or with an antigen or other molecule attached to the complex or nanostructure by other means, such as bioconjugate chemistry. In some embodiments, the alpha-helical segments described herein are used as fusion proteins to monomeric antigens, including but not limited to the receptor binding domain (RBD) of the SARS-COV-2 spike(S) protein.

The protein nanostructures of the present invention may comprise multimeric protein assemblies adapted for display of molecules such as antigens (e.g., engineered ectodomains). The protein nanostructures, in some embodiments described herein, comprise at least a first component displaying an engineered ectodomain and, optionally, a second component. The engineered ectodomain may include one or more amino acid substitutions, a C-terminal helix-forming segment, or a combination thereof. The first component may comprise or consist of three copies of a fusion protein. In some embodiments, the fusion protein comprises an assembly domain having a protein sequence designed by computational methods to assemble to form a nanostructure. In some embodiments, the first component is a trimeric component in which the assembly domains form trimers related by 3-fold rotational symmetry, and/or the second component is a pentameric component, in which the assembly domains form pentamers related by 5-fold rotational symmetry. In some embodiments, the combination of the two components form an “icosahedral particle” having 153 symmetry. Together these components may be arranged such that the members of each component are related to one another by symmetry operators. A general computational method for designing self-assembling protein materials, involving symmetrical docking of protein building blocks in a target symmetric architecture, is disclosed in Patent Pub. No. US 2015/0356240 A1.

The “core” of the protein nanostructure is used herein to describe the central portion of the protein nanostructure. For clarity, the term “core” as used herein excludes molecules displayed by the nanostructure. The core may serve to assemble multiple copies of the displayed molecule, such as an antigen (e.g., an engineered ectodomain). Without being bound by theory, this may increase the immunogenicity of an antigen. The disclosure envisions nanostructures in which the core is either non-covalently associated with the displayed antigen; covalently linked to the display antigen (such as by chemical conjugation); or, in preferred embodiments, linked to the displayed antigen through a polypeptide linker in a fusion protein. In some embodiments, the fusion protein comprises a first polypeptide comprising an antigen (e.g., an ectodomain), and a first assembly domain. In some embodiments, an antigen (e.g., an ectodomain) is non-covalently or covalently linked to the assembly domain. For example, an antigen (e.g., an ectodomain) may be fused to the first component and configured to bind a portion of the first component, or a chemical tag on the first component. For example, a streptavidin-biotin (or neutravidin-biotin) linker can be employed. Alternatively, various bioconjugate linkers may be used. In some embodiments of the present disclosure, the antigen comprises further polypeptide sequences in addition to RSV F protein.

In some embodiments, three copies of an antigen (e.g., an ectodomain) polypeptide are displayed on a 3-fold axis. Thus, the protein nanostructure is capable of displaying 60 monomeric antigen (e.g., an ectodomain) polypeptides. In some embodiments, the protein nanostructure is adapted for display of up to 12, 24, or 60 monomers. In some embodiments, a component may comprise a polypeptide linked to diverse engineered ectodomains, such that the protein nanostructure displays different ectodomains on the same nanostructure. In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more different ectodomains are displayed. Non-limiting illustrative protein nanostructure are provided in Bale et al. Science 353:389-94 (2016); Heinze et al. J. Phys. Chem B. 120:5945-5952 (2016); King et al. Nature 510:103-108 (2014); and King et al. Science 336:1171-71 (2012).

### Attachment Modalities

The protein nanostructures of the present disclosure display antigenic proteins in various ways including as gene fusion or by other means disclosed herein. As used herein, “linked to” or “attached to” denotes any means known in the art for causing two polypeptides to associate. The association may be direct or indirect, reversible or irreversible, weak or strong, covalent or non-covalent, and selective or nonselective.

In some embodiments, attachment is achieved by genetic engineering to create an N- or C-terminal fusion of potentially antigenic polypeptides of the protein nanostructure.

In some embodiments, attachment is achieved by post-translational covalent attachment of one or more pluralities of antigenic protein. In some embodiments, chemical cross-linking is used to non-specifically attach the antigen to a protein nanostructure. In some embodiments, chemical cross-linking is used to specifically attach the antigenic protein to a protein nanostructure (e.g., to the first polypeptide or the second polypeptide). Various specific and non-specific cross-linking chemistries are known in the art, such as Click chemistry and other methods. In general, any cross-linking chemistry/bioconjugate used to link two proteins may be adapted for use in the presently disclosed protein nanostructures. In particular, chemistries used in creation of immunoconjugates or antibody drug conjugates may be used. In some embodiments, a protein nanostructure is created using a cleavable or non-cleavable linker. Processes and methods for conjugation of antigens to carriers are provided by, e.g., Patent Pub. No. US 2008/0145373 A1.

The protein nanostructures may employ a variety of coupling techniques to attach an antigen to the core, including but not limited to the SpyCatcher system described in, e.g., Escolano et al. Nature 570:468-473 (2019), He et al. Sci Adv. 7 (12):eabf1591 (2021), and Tan et al. Nat. Commun. 12 (1): 542 (2021).

In some embodiments, attachment is achieved by non-covalent attachment between a component and the ectodomain. In some embodiments the ectodomain is engineered to be negatively charged on at least one surface and the core polypeptide is engineered to be positively charged on at least one surface, or positively and negatively charged, respectively. This can promote intermolecular association between the ectodomain and the component core polypeptide by electrostatic force. In some embodiments, shape complementarity is employed to cause linkage of ectodomain to component core. Shape complementarity can be pre-existing or rationally designed. In some embodiments, computational design of protein-protein interfaces is used to achieve attachment.

In another aspect, the disclosure provides a trimeric protein complex comprising a polypeptide disclosed herein. In another aspect, the disclosure provides a protein nanostructure comprising a trimeric component comprising a polypeptide disclosed herein.

In some embodiments, the nanostructure is a two-component nanostructure comprising the first, trimeric component and a second, pentameric component. In some embodiments, the first, trimeric component comprises an engineered ectodomain of a Respiratory Syncytial Virus (RSV) fusion (F) polypeptide and an I53-50A polypeptide. In some embodiments the first, trimeric component comprises an engineered ectodomain of a human Metapneumovirus (hMPV) fusion (F) polypeptide and an I53-50A polypeptide. In some embodiments the first, trimeric component comprises an engineered ectodomain of a human Parainfluenza virus type 3 (PIV3) fusion (F) polypeptide and an I53-50A polypeptide. In some embodiments the first, trimeric component comprises an engineered ectodomain of a human Parainfluenza virus type 5 (PIV3) fusion (F) polypeptide and an I53-50A polypeptide. In some embodiments the first, trimeric component comprises an engineered ectodomain of a SARS-COV-2 spike(S) polypeptide and an I53-50A polypeptide. In some embodiments the first, trimeric component comprises an engineered ectodomain of a Nipaha virus fusion (F) polypeptide and an I53-50A polypeptide. In some embodiments the first, trimeric component comprises a fusion protein comprising, in N- to C-terminal order, the engineered fusion (F) polypeptide, an amino acid linker, and the I53-50A polypeptide. In some embodiments the first, trimeric component comprises a fusion protein comprising, in N- to C-terminal order, the engineered spike(S) polypeptide, an amino acid linker, and the I53-50A polypeptide. In some embodiments, the trimeric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of the sequences listed in Table 19 or to any one of the sequences listed in Table 19 without the underlined and/or bold/italicized polypeptide sequences. In some embodiments the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 20, 44, 45, 52, 71, 73, 74.

### Polypeptide Sequences

Patent Pub No. US 2015/0356240 A1 describes various methods for designing protein assemblies. As described in US Patent Pub No. US 2016/0122392 A1 and in International Patent Pub. No. WO 2014/124301 A1, the isolated polypeptides of SEQ ID NOs: 13-63 were designed for their ability to self-assemble in pairs to form protein nanostructures, such as icosahedral particles. The design involved design of suitable interface residues for each member of the polypeptide pair that can be assembled to form the protein nanostructures. The protein nanostructures so formed include symmetrically repeated, non-natural, non-covalent polypeptide-polypeptide interfaces that orient a first assembly domain and a second assembly domain into protein nanostructures, such as one with an icosahedral symmetry. Thus, in one embodiment a first assembly domain and second assembly domain of the component are selected from the group consisting of SEQ ID NOs: 13-63. In each case, an N-terminal methionine residue present in the full length protein is included, but may be removed to make a fusion that is not included in the sequence. The identified residues in Table 11 are numbered beginning with an N-terminal methionine (not shown). In various embodiments, one or more additional residues are deleted from the N-terminus and/or additional residues are added to the N-terminus (e.g., to form a helical extension).

TABLE 11

| | | | Identified |
|---|---|---|---|
| | Component | | interface |
| Name | Multimer | Amino Acid Sequence | residues |
| I53-34A | trimer | EGMDPLAVLAESRLLPLLTVRGGEDLAGLATVLELMGV | I53-34A: |
| SEQ ID | | GALEITLRTEKGLEALKALRKSGLLLGAGTVRSPKEAE | 28, 32, 36, |
| NO: 13 | | AALEAGAAFLVSPGLLEEVAALAQARGVPYLPGVLTPT | 37, 186, |
| | | EVERALALGLSALKFFPAEPFQGVRVLRAYAEVFPEVR | 188, 191, |
| | | FLPTGGIKEEHLPHYAALPNLLAVGGSWLLQGDLAAVM | 192, 195 |
| | | KKVKAAKALLSPQAPG | |
| I53-34B | pentamer | TKKVGIVDTTFARVDMAEAAIRTLKALSPNIKIIRKTV | I53-34B: |
| SEQ ID | | PGIKDLPVACKKLLEEEGCDIVMALGMPGKAEKDKVCA | 19, 20, 23, |
| NO: 14 | | HEASLGLMLAQLMTNKHIIEVFVHEDEAKDDDELDILA | 24, 27, 109, |
| | | LVRAIEHAANVYYLLFKPEYLTRMAGKGLRQGREDAGP | 113, 116, |
| | | ARE | 117, 120, |
| | | | 124, 148 |
| I53-40A | pentamer | TKKVGIVDTTFARVDMASAAILTLKMESPNIKIIRKTV | I53-40A: |
| SEQ ID | | PGIKDLPVACKKLLEEEGCDIVMALGMPGKAEKDKVCA | 20, 23, 24, |
| NO: 15 | | HEASLGLMLAQLMTNKHIIEVFVHEDEAKDDAELKILA | 27, 28, 109, |
| | | ARRAIEHALNVYYLLFKPEYLTRMAGKGLRQGFEDAGP | 112, 113, |
| | | ARE | 116, 120, |
| | | | 124 |
| I53-40B | trimer | STINNQLKALKVIPVIAIDNAEDIIPLGKVLAENGLPA | I53-40B: |
| SEQ ID | | AEITFRSSAAVKAIMLLRSAQPEMLIGAGTILNGVQAL | 47, 51, 54, |
| NO: 16 | | AAKEAGATFVVSPGFNPNTVRACQIIGIDIVPGVNNPS | 58, 74, 102 |
| | | TVEAALEMGLTTLKFFPAEASGGISMVKSLVGPYGDIR | |
| | | LMPTGGITPSNIDNYLAIPQVLACGGTWMVDKKLVTNG | |
| | | EWDEIARLTREIVEQVNP | |
| I53-47A | trimer | PIFTLNTNIKATDVPSDFLSLTSRLVGLILSKPGSYVA | I53-47A: |
| SEQ ID | | VHINTDQQLSFGGSTNPAAFGTLMSIGGIEPSKNRDHS | 22, 25, 29, |
| NO: 17 | | AVLFDHLNAMLGIPKNRMYIHFVNLNGDDVGWNGTTF | 72, 79, 86, |
| | | | 87 |
| I53-47B | pentamer | NQHSHKDYETVRIAVVRARWHADIVDACVEAFEIAMAA | I53-47B: |
| SEQ ID | | IGGDRFAVDVFDVPGAYEIPLHARTLAETGRYGAVLGT | 28, 31, 35, |
| NO: 18 | | AFVVNGGIYRHEFVASAVIDGMMNVQLSTGVPVLSAVL | 36, 39, 131, |
| | | TPHRYRDSAEHHRFFAAHFAVKGVEAARACIEILAARE | 132, 135, |
| | | KIAA | 139, 146 |
| I53-50A | trimer | EELFKKHKIVAVLRANSVEEAIEKAVAVFAGGVHLIEI | I53-50A: |
| SEQ ID | | TFTVPDADTVIKALSVLKEKGAIIGAGTVTSVEQCRKA | 25, 29, 33, |
| NO: 19 | | VESGAEFIVSPHLDEEISQFCKEKGVFYMPGVMTPTEL | 54, 57 |
| | | VKAMKLGHTILKLFPGEVVGPQFVKAMKGPFPNVKFVP | |
| | | TGGVNLDNVCEWFKAGVLAVGVGSALVKGTPDEVREKA | |
| | | KAFVEKIRGCTE | |
| I53-50B | pentamer | NQHSHKDYETVRIAVVRARWHAEIVDACVSAFEAAMAD | I53-50B: |
| SEQ ID | | IGGDRFAVDVFDVPGAYEIPLHARTLAETGRYGAVLGT | 24, 28, 36, |
| NO: 20 | | AFVVNGGIYRHEFVASAVIDGMMNVQLSTGVPVLSAVL | 124, 125, |
| | | TPHRYRDSDAHTLLFLALFAVKGMEAARACVEILAARE | 127, 128, |
| | | KIAA | 129, 131, |
| | | | 132, 133, |
| | | | 135, 139 |
| I53-51A | trimer | FTKSGDDGNTNVINKRVGKDSPLVNFLGDLDELNSFIG | I53-51A: |
| SEQ ID | | FAISKIPWEDMKKDLERVQVELFEIGEDLSTQSSKKKI | 80, 83, 86, |
| NO: 21 | | DESYVLWLLAATAIYRIESGPVKLFVIPGGSEEASVLH | 87, 88, 90, |
| | | VTRSVARRVERNAVKYTKELPEINRMIIVYLNRLSSLL | 91, 94, 166, |
| | | FAMALVANKRRNQSEKIYEIGKSW | 172, 176 |
| I53-51B | pentamer | NQHSHKDYETVRIAVVRARWHADIVDQCVRAFEEAMAD | I53-51B: |
| SEQ ID | | AGGDRFAVDVFDVPGAYEIPLHARTLAETGRYGAVLGT | 31, 35, 36, |
| NO: 22 | | AFVVNGGIYRHEFVASAVIDGMMNVQLSTGVPVLSAVL | 40, 122, |
| | | TPHRYRSSREHHEFFREHFMVKGVEAAAACITILAARE | 124, 128, |
| | | KIAA | 131, 135, |
| | | | 139, 143, |
| | | | 146, 147 |
| I52-03A | pentamer | GHTKGPTPQQHDGSALRIGIVHARWNKTIIMPLLIGTI | I52-03A: |
| SEQ ID | | AKLLECGVKASNIVVQSVPGSWELPIAVQRLYSASQLQ | 28, 32, 36, |
| NO: 23 | | TPSSGPSLSAGDLLGSSTTDLTALPTTTASSTGPFDAL | 39, 44, 49 |
| | | IAIGVLIKGETMHFEYIADSVSHGLMRVQLDTGVPVIF | |
| | | GVLTVLTDDQAKARAGVIEGSHNHGEDWGLAAVEMGVR | |
| | | RRDWAAGKTE | |
| I52-03B | dimer | YEVDHADVYDLFYLGRGKDYAAEASDIADLVRSRTPEA | I52-03B: |
| SEQ ID | | SSLLDVACGTGTHLEHFTKEFGDTAGLELSEDMLTHAR | 94, 115, |
| NO: 24 | | KRLPDATLHQGDMRDFQLGRKFSAVVSMFSSVGYLKTV | 116, 206, |
| | | AELGAAVASFAEHLEPGGVVVVEPWWFPETFADGWVSA | 213 |
| | | DVVRRDGRTVARVSHSVREGNATRMEVHFTVADPGKGV | |
| | | RHFSDVHLITLFHQREYEAAFMAAGLRVEYLEGGPSGR | |
| | | GLFVGVPA | |
| I52-32A | dimer | GMKEKFVLIITHGDFGKGLLSGAEVIIGKQENVHTVGL | I52-32A: |
| SEQ ID | | NLGDNIEKVAKEVMRIIIAKLAEDKEIIIVVDLFGGSP | 47, 49, 53, |
| NO: 25 | | FNIALEMMKTFDVKVITGINMPMLVELLTSINVYDTTE | 54, 57, 58, |
| | | LLENISKIGKDGIKVIEKSSLKM | 61, 83, 87, |
| | | | 88 |
| I52-32B | pentamer | KYDGSKLRIGILHARWNLEIIAALVAGAIKRLQEFGVK | I52-32B: |
| SEQ ID | | AENIIIETVPGSFELPYGSKLFVEKQKRLGKPLDAIIP | 19, 20, 23, |
| NO: 26 | | IGVLIKGSTMHFEYICDSTTHQLMKLNFELGIPVIFGV | 30, 40 |
| | | LTCLTDEQAEARAGLIEGKMHNHGEDWGAAAVEMATKF | |
| | | N | |
| I52-33A | pentamer | AVKGLGEVDQKYDGSKLRIGILHARWNRKIILALVAGA | I52-33A: |
| SEQ ID | | VLRLLEFGVKAENIIIETVPGSFELPYGSKLFVEKQKR | 33, 41, 44, |
| NO: 27 | | LGKPLDAIIPIGVLIKGSTMHFEYICDSTTHQLMKLNF | 50 |
| | | ELGIPVIFGVLTCLTDEQAEARAGLIEGKMHNHGEDWG | |
| | | AAAVEMATKFN | |
| I52-33B | dimer | GANWYLDNESSRLSFTSTKNADIAEVHRFLVLHGKVDP | I52-33B: |
| SEQ ID | | KGLAEVEVETESISTGIPLRDMLLRVLVFQVSKFPVAQ | 61, 63, 66, |
| NO: 28 | | INAQLDMRPINNLAPGAQLELRLPLTVSLRGKSHSYNA | 67, 72, 147, |
| | | ELLATRLDERRFQVVTLEPLVIHAQDFDMVRAFNALRL | 148, 154, |
| | | VAGLSAVSLSVPVGAVLIFTAR | 155 |
| I32-06A | dimer | TDYIRDGSAIKALSFAIILAEADLRHIPQDLQRLAVRV | I32-06A: |
| SEQ ID | | IHACGMVDVANDLAFSEGAGKAGRNALLAGAPILCDAR | 9, 12, 13, |
| NO: 29 | | MVAEGITRSRLPADNRVIYTLSDPSVPELAKKIGNTRS | 14, 20, 30, |
| | | AAALDLWLPHIEGSIVAIGNAPTALFRLFELLDAGAPK | 33, 34 |
| | | PALIIGMPVGFVGAAESKDELAANSRGVPYVIVRGRRG | |
| | | GSAMTAAAVNALASERE | |
| I32-06B | trimer | ITVFGLKSKLAPRREKLAEVIYSSLHLGLDIPKGKHAI | I32-06B: |
| SEQ ID | | RFLCLEKEDFYYPFDRSDDYTVIEINLMAGRSEETKML | 24, 71, 73, |
| NO: 30 | | LIFLLFIALERKLGIRAHDVEITIKEQPAHCWGFRGRT | 76, 77, 80, |
| | | GDSARDLDYDIYV | 81, 84, 85, |
| | | | 88, 114, |
| | | | 118 |
| I32-19A | trimer | GSDLQKLQRFSTCDISDGLLNVYNIPTGGYFPNLTAIS | I32-19A: |
| SEQ ID | | PPQNSSIVGTAYTVLFAPIDDPRPAVNYIDSVPPNSIL | 208, 213, |
| NO: 31 | | VLALEPHLQSQFHPFIKITQAMYGGLMSTRAQYLKSNG | 218, 222, |
| | | TVVFGRIRDVDEHRTLNHPVFAYGVGSCAPKAVVKAVG | 225, 226, |
| | | TNVQLKILTSDGVTQTICPGDYIAGDNNGIVRIPVQET | 229, 233 |
| | | DISKLVTYIEKSIEVDRLVSEAIKNGLPAKAAQTARRM | |
| | | VLKDYI | |
| I32-19B | dimer | SGMRVYLGADHAGYELKQAIIAFLKMTGHEPIDCGALR | I32-19B: |
| SEQ ID | | YDADDDYPAFCIAAATRTVADPGSLGIVLGGSGNGEQI | 20, 23, 24, |
| NO: 32 | | AANKVPGARCALAWSVQTAALAREHNNAQLIGIGGRMH | 27, 117, |
| | | TLEEALRIVKAFVTTPWSKAQRHQRRIDILAEYERTHE | 118, 122, |
| | | APPVPGAPA | 125 |
| I32-28A | trimer | GDDARIAAIGDVDELNSQIGVLLAEPLPDDVRAALSAI | I32-28A: |
| SEQ ID | | QHDLFDLGGELCIPGHAAITEDHLLRLALWLVHYNGQL | 60, 61, 64, |
| NO: 33 | | PPLEEFILPGGARGAALAHVCRTVCRRAERSIKALGAS | 67, 68, 71, |
| | | EPLNIAPAAYVNLLSDLLFVLARVLNRAAGGADVLWDR | 110, 120, |
| | | TRAH | 123, 124, |
| | | | 128 |
| I32-28B | dimer | ILSAEQSFTLRHPHGQAAALAFVREPAAALAGVQRLRG | I32-28B: |
| SEQ ID | | LDSDGEQVWGELLVRVPLLGEVDLPFRSEIVRTPQGAE | 35, 36, 54, |
| NO: 34 | | LRPLTLTGERAWVAVSGQATAAEGGEMAFAFQFQAHLA | 122, 129, |
| | | TPEAEGEGGAAFEVMVQAAAGVTLLLVAMALPQGLAAG | 137, 140, |
| | | LPPA | 141, 144, |
| | | | 148 |
| I53-40A.1 | pentamer | TKKVGIVDTTFARVDMASAAILTLKMESPNIKIIRKTV | I53-40A: |
| SEQ ID | | PGIKDLPVACKKLLEEEGCDIVMALGMPGKKEKDKVCA | 20, 23, 24, |
| NO: 35 | | HEASLGLMLAQLMTNKHIIEVFVHEDEAKDDAELKILA | 27, 28, 109, |
| | | ARRAIEHALNVYYLLFKPEYLTRMAGKGLRQGFEDAGP | 112, 113, |
| | | ARE | 116, 120, |
| | | | 124 |
| I53-40B.1 | trimer | DDINNQLKRLKVIPVIAIDNAEDIIPLGKVLAENGLPA | I53-40B: |
| SEQ ID | | AEITFRSSAAVKAIMLLRSAQPEMLIGAGTILNGVQAL | 47, 51, 54, |
| NO: 36 | | AAKEAGADFVVSPGFNPNTVRACQIIGIDIVPGVNNPS | 58, 74, 102 |
| | | TVEQALEMGLTTLKFFPAEASGGISMVKSLVGPYGDIR | |
| | | LMPTGGITPDNIDNYLAIPQVLACGGTWMVDKKLVRNG | |
| | | EWDEIARLTREIVEQVNP | |
| I53-47A.1 | trimer | PIFTLNTNIKADDVPSDFLSLTSRLVGLILSKPGSYVA | I53-47A: |
| SEQ ID | | VHINTDQQLSFGGSTNPAAFGTLMSIGGIEPDKNRDHS | 22, 25, 29, |
| NO: 37 | | AVLFDHLNAMLGIPKNRMYIHFVNLNGDDVGWNGTTF | 72, 79, 86, |
| | | | 87 |
| I53- | trimer | PIFTLNTNIKADDVPSDFLSLTSRLVGLILSEPGSYVA | I53-47A: |
| 47A.1NegT | | VHINTDQQLSFGGSTNPAAFGTLMSIGGIEPDKNEDHS | 22, 25, 29, |
| 2 | | AVLFDHLNAMLGIPKNRMYIHFVDLDGDDVGWNGTTF | 72, 79, 86, |
| SEQ ID | | | 87 |
| NO: 38 | | | |
| I53-47B.1 | pentamer | NQHSHKDHETVRIAVVRARWHADIVDACVEAFEIAMAA | I53-47B: |
| SEQ ID | | IGGDRFAVDVEDVPGAYEIPLHARTLAETGRYGAVLGT | 28, 31, 35, |
| NO: 39 | | AFVVNGGIYRHEFVASAVIDGMMNVQLDTGVPVLSAVL | 36, 39, 131, |
| | | TPHRYRDSDEHHRFFAAHFAVKGVEAARACIEILNARE | 132, 135, |
| | | KIAA | 139, 146 |
| I53- | pentamer | NQHSHKDHETVRIAVVRARWHADIVDACVEAFEIAMAA | I53-47B: |
| 47B.1NegT | | IGGDRFAVDVFDVPGAYEIPLHARTLAETGRYGAVLGT | 28, 31, 35, |
| 2 | | AFVVDGGIYDHEFVASAVIDGMMNVQLDTGVPVLSAVL | 36, 39, 131, |
| SEQ ID | | TPHEYEDSDEDHEFFAAHFAVKGVEAARACIEILNARE | 132, 135, |
| NO: 40 | | KIAA | 139, 146 |
| I53-50A.1 | trimer | EELFKKHKIVAVLRANSVEEAIEKAVAVFAGGVHLIEI | I53-50A: |
| SEQ ID | | TFTVPDADTVIKALSVLKEKGAIIGAGTVTSVEQCRKA | 25, 29, 33, |
| NO: 41 | | VESGAEFIVSPHLDEEISQFCKEKGVFYMPGVMTPTEL | 54, 57 |
| | | VKAMKLGHDILKLFPGEVVGPQFVKAMKGPFPNVKFVP | |
| | | TGGVNLDNVCEWFKAGVLAVGVGDALVKGDPDEVREKA | |
| | | KKFVEKIRGCTE | |
| I53- | trimer | EELFKKHKIVAVLRANSVEEAIEKAVAVFAGGVHLIEI | I53-50A: |
| 50A.1NegT | | TFTVPDADTVIKALSVLKEKGAIIGAGTVTSVEQCRKA | 25, 29, 33, |
| 2 | | VESGAEFIVSPHLDEEISQFCKEKGVFYMPGVMTPTEL | 54, 57 |
| SEQ ID | | VKAMKLGHDILKLFPGEVVGPEFVEAMKGPFPNVKFVP | |
| NO: 42 | | TGGVDLDDVCEWFDAGVLAVGVGDALVEGDPDEVREDA | |
| | | KEFVEEIRGCTE | |
| I53- | trimer | EELFKKHKIVAVLRANSVEEAIEKAVAVFAGGVHLIEI | I53-50A: |
| 50A.1PosT | | TFTVPDADTVIKALSVLKEKGAIIGAGTVTSVEQCRKA | 25, 29, 33, |
| 1 | | VESGAEFIVSPHLDEEISQFCKEKGVFYMPGVMTPTEL | 54, 57 |
| SEQ ID | | VKAMKLGHDILKLFPGEVVGPQFVKAMKGPFPNVKFVP | |
| NO: 43 | | TGGVNLDNVCKWFKAGVLAVGVGKALVKGKPDEVREKA | |
| | | KKFVKKIRGCTE | |
| I53-50B.1 | pentamer | NQHSHKDHETVRIAVVRARWHAEIVDACVSAFEAAMRD | I53-50B: |
| SEQ ID | | IGGDRFAVDVEDVPGAYEIPLHARTLAETGRYGAVLGT | 24, 28, 36, |
| NO: 44 | | AFVVNGGIYRHEFVASAVIDGMMNVQLDTGVPVLSAVL | 124, 125, |
| | | TPHRYRDSDAHTLLELALFAVKGMEAARACVEILAARE | 127, 128, |
| | | KIAA | 129, 131, |
| | | | 132, 133, |
| | | | 135, 139 |
| I53- | pentamer | NQHSHKDHETVRIAVVRARWHAEIVDACVSAFEAAMRD | I53-50B: |
| 50B.1NegT | | IGGDRFAVDVFDVPGAYEIPLHARTLAETGRYGAVLGT | 24, 28, 36, |
| 2 | | AFVVDGGIYDHEFVASAVIDGMMNVQLDTGVPVLSAVL | 124, 125, |
| SEQ ID | | TPHEYEDSDADTLLFLALFAVKGMEAARACVEILAARE | 127, 128, |
| NO: 45 | | KIAA | 129, 131, |
| | | | 132, 133, |
| | | | 135, 139 |
| I53- | trimer | NQHSHKDHETVRIAVVRARWHAEIVDACVSAFEAAMRD | I53-50B: |
| 50B.4PosT | | IGGDRFAVDVEDVPGAYEIPLHARTLAETGRYGAVLGT | 24, 28, 36, |
| 1 | | AFVVNGGIYRHEFVASAVINGMMNVQLNTGVPVLSAVL | 124, 125, |
| SEQ ID | | TPHNYDKSKAHTLLFLALFAVKGMEAARACVEILAARE | 127, 128, |
| NO: 46 | | KIAA | 129, 131, |
| | | | 132, 133, |
| | | | 135, 139 |
| I53-40A | pentamer | TKKVGIVDTTFARVDMASAAILTLKMESPNIKIIRKTV | |
| genus | | PGIKDLPVACKKLLEEEGCDIVMALGMPGK(A/K)EKD | |
| SEQ ID | | KVCAHEASLGLMLAQLMTNKHIIEVFVHEDEAKDDAEL | |
| NO: 47 | | KILAARRAIEHALNVYYLLEKPEYLTRMAGKGLRQGFE | |
| | | DAGPARE | |
| I53-40B | trimer | (S/D)(T/D)INNQLK(A/R)LKVIPVIAIDNAEDIIP | |
| genus | | LGKVLAENGLPAAEITFRSSAAVKAIMLLRSAQPEMLI | |
| SEQ ID | | GAGTILNGVQALAAKEAGA(T/D)FVVSPGFNPNTVRA | |
| NO: 48 | | CQIIGIDIVPGVNNPSTVE(A/Q)ALEMGLTTLKFFPA | |
| | | EASGGISMVKSLVGPYGDIRLMPTGGITP(S/D)NIDN | |
| | | YLAIPQVLACGGTWMVDKKLV(T/R)NGEWDEIARLTR | |
| | | EIVEQVNP | |
| I53-47A | trimer | PIFTLNTNIKA(T/D)DVPSDFLSLTSRLVGLILS(K/ | |
| genus | | E)PGSYVAVHINTDQQLSFGGSTNPAAFGTLMSIGGIE | |
| SEQ ID | | P(S/D)KN(R/E)DHSAVLFDHLNAMLGIPKNRMYIHF | |
| NO: 49 | | V(N/D)L(N/D)GDDVGWNGTTF | |
| I53-47B | pentamer | NQHSHKD(Y/H)ETVRIAVVRARWHADIVDACVEAFEI | |
| genus | | AMAAIGGDRFAVDVFDVPGAYEIPLHARTLAETGRYGA | |
| SEQ ID | | VLGTAFVV(N/D)GGIY(R/D)HEFVASAVIDGMMNVQ | |
| NO: 50 | | L(S/D)TGVPVLSAVLTPH(R/E)Y(R/E)DS(A/D)E | |
| | | (H/D)H(R/E)FFAAHFAVKGVEAARACIEIL(A/N)A | |
| | | REKIAA | |
| I53-50A | trimer | EELFKKHKIVAVLRANSVEEAIEKAVAVFAGGVHLIEI | |
| genus | | TFTVPDADTVIKALSVLKEKGAIIGAGTVTSVEQCRKA | |
| SEQ ID | | VESGAEFIVSPHLDEEISQFCKEKGVFYMPGVMTPTEL | |
| NO: 51 | | VKAMKLGH(T/D)ILKLFPGEVVGP(Q/E)FV(K/E)A | |
| | | MKGPFPNVKFVPTGGV(N/D)LD(N/D)VC(E/K)WF | |
| | | (K/D)AGVLAVGVG(S/K/D)ALV(K/E)G(T/D/K)P | |
| | | DEVRE(K/D)AK(A/E/K)FV(E/K)(K/E)IRGCTE | |
| I53-50B | pentamer | NQHSHKD(Y/H)ETVRIAVVRARWHAEIVDACVSAFEA | |
| genus | | AM(A/R)DIGGDRFAVDVFDVPGAYEIPLHARTLAETG | |
| SEQ ID | | RYGAVLGTAFVV(N/D)GGIY(R/D)HEFVASAVI(D/ | |
| NO: 52 | | N)GMMNVQL(S/D/N)TGVPVLSAVLTPH(R/E/N)Y | |
| | | (R/D/E)(D/K)S(D/K)A(H/D)TLLFLALFAVKGME | |
| | | AARACVEILAAREKIAA | |
| T32-28A | dimer | GEVPIGDPKELNGMEIAAVYLQPIEMEPRGIDLAASLA | |
| SEQ ID | | DIHLEADIHALKNNPNGFPEGEWMPYLTIAYALANADT | |
| NO: 53 | | GAIKTGTLMPMVADDGPHYGANIAMEKDKKGGFGVGTY | |
| | | ALTFLISNPEKQGFGRHVDEETGVGKWFEPFVVTYFFK | |
| | | YTGTPK | |
| T32-28B | trimer | SQAIGILELTSIAKGMELGDAMLKSANVDLLVSKTISP | |
| SEQ ID | | GKFLLMLGGDIGAIQQAIETGTSQAGEMLVDSLVLANI | |
| NO: 54 | | HPSVLPAISGLNSVDKRQAVGIVETWSVAACISAADLA | |
| | | VKGSNVTLVRVHMAFGIGGKCYMVVAGDVLDVAAAVAT | |
| | | ASLAAGAKGLLVYASIIPRPHEAMWRQMVEG | |
| T33-09A | trimer | EEVVLITVPSALVAVKIAHALVEERLAACVNIVPGLTS | |
| SEQ ID | | IYRWQGSVVSDHELLLLVKTTTHAFPKLKERVKALHPY | |
| NO: 55 | | TVPEIVALPIAEGNREYLDWLRENTG | |
| T33-09B | trimer | VRGIRGAITVEEDTPAAILAATIELLLKMLEANGIQSY | |
| SEQ ID | | EELAAVIFTVTEDLTSAFPAEAARLIGMHRVPLLSARE | |
| NO: 56 | | VPVPGSLPRVIRVLALWNTDTPQDRVRHVYLNEAVRLR | |
| | | PDLESAQ | |
| T33-15A | trimer | SKAKIGIVTVSDRASAGITADISGKAIILALNLYLTSE | |
| SEQ ID | | WEPIYQVIPDEQDVIETTLIKMADEQDCCLIVTTGGTG | |
| NO: 57 | | PAKRDVTPEATEAVCDRMMPGFGELMRAESLKEVPTAI | |
| | | LSRQTAGLRGDSLIVNLPGDPASISDCLLAVFPAIPYC | |
| | | IDLMEGPYLECNEAMIKPERPKAK | |
| T33-15B | trimer | VRGIRGAITVNSDTPTSIIIATILLLEKMLEANGIQSY | |
| SEQ ID | | EELAAVIFTVTEDLTSAFPAEAARQIGMHRVPLLSARE | |
| NO: 58 | | VPVPGSLPRVIRVLALWNTDTPQDRVRHVYLSEAVRLR | |
| | | PDLESAQ | |
| T33-21A | trimer | RITTKVGDKGSTRLFGGEEVWKDSPIIEANGTLDELTS | |
| SEQ ID | | FIGEAKHYVDEEMKGILEEIQNDIYKIMGEIGSKGKIE | |
| NO: 59 | | GISEERIAWLLKLILRYMEMVNLKSFVLPGGTLESAKL | |
| | | DVCRTIARRALRKVLTVTREFGIGAEAAAYLLALSDLL | |
| | | FLLARVIEIEKNKLKEVRS | |
| T33-21B | trimer | PHLVIEATANLRLETSPGELLEQANKALFASGQFGEAD | |
| SEQ ID | | IKSRFVTLEAYRQGTAAVERAYLHACLSILDGRDIATR | |
| NO: 60 | | TLLGASLCAVLAEAVAGGGEEGVQVSVEVREMERLSYA | |
| | | KRVVARQR | |
| T33-28A | trimer | ESVNTSFLSPSLVTIRDFDNGQFAVLRIGRTGFPADKG | |
| SEQ ID | | DIDLCLDKMIGVRAAQIFLGDDTEDGFKGPHIRIRCVD | |
| NO: 61 | | IDDKHTYNAMVYVDLIVGTGASEVERETAEEEAKLALR | |
| | | VALQVDIADEHSCVTQFEMKLREELLSSDSFHPDKDEY | |
| | | YKDFL | |
| T33-28B | trimer | PVIQTFVSTPLDHHKRLLLAIIYRIVTRVVLGKPEDLV | |
| SEQ ID | | MMTFHDSTPMHFFGSTDPVACVRVEALGGYGPSEPEKV | |
| NO: 62 | | TSIVTAAITAVCGIVADRIFVLYFSPLHCGWNGTNF | |
| T33-31A | trimer | EEVVLITVPSALVAVKIAHALVEERLAACVNIVPGLTS | |
| SEQ ID | | IYREEGSVVSDHELLLLVKTTTDAFPKLKERVKELHPY | |
| NO: 63 | | EVPEIVALPIAEGNREYLDWLRENTG | |
| I53-50A | trimer | EELFKKHKIVAVLRANSVEEAIEKAVAVFAGGVHLIEI | |
| ΔCys | | TFTVPDADTVIKALSVLKEKGAIIGAGTVTSVEQARKA | |
| SEQ ID | | VESGAEFIVSPHLDEEISQFAKEKGVFYMPGVMTPTEL | |
| NO: 64 | | VKAMKLGHTILKLFPGEVVGPQFVKAMKGPFPNVKFVP | |
| | | TGGVNLDNVAEWFKAGVLAVGVGSALVKGTPDEVREKA | |
| | | KAFVEKIRGATE | |
| T33_dn2A | | NLAEKMYKAGNAMYRKGQYTIAIIAYTLALLKDPNNAE | |
| SEQ ID | | AWYNLGNAAYKKGEYDEAIEAYQKALELDPNNAEAWYN | |
| NO: 65 | | LGNAYYKQGDYDEAIEYYKKALRLDPRNVDAIENLIEA | |
| | | EEKQG | |
| T33_dn2B | | EEAELAYLLGELAYKLGEYRIAIRAYRIALKRDPNNAE | |
| SEQ ID | | AWYNLGNAYYKQGDYREAIRYYLRALKLDPENAEAWYN | |
| NO: 66 | | LGNALYKQGKYDLAIIAYQAALEEDPNNAEAKQNLGNA | |
| | | KQKQG | |
| T33_dn5A | | NSAEAMYKMGNAAYKQGDYILAIIAYLLALEKDPNNAE | |
| SEQ ID | | AWYNLGNAAYKQGDYDEAIEYYQKALELDPNNAEAWYN | |
| NO: 67 | | LGNAYYKQGDYDEAIEYYEKALELDPNNAEALKNLLEA | |
| | | IAEQD | |
| T33 dn5A | | TDPLAVILYIAILKAEKSIARAKAAEALGKIGDERAVE | |
| SEQ ID | | PLIKALKDEDALVRAAAADALGQIGDERAVEPLIKALK | |
| NO: 68 | | DEEGLVRASAAIALGQIGDERAVQPLIKALTDERDLVR | |
| | | VAAAVALGRIGDEKAVRPLIIVLKDEEGEVREAAAIAL | |
| | | GSIGGERVRAAMEKLAERGTGFARKVAVNYLETHK | |
| T33_dn10A | | EEAELAYLLGELAYKLGEYRIAIRAYRIALKRDPNNAE | |
| SEQ ID | | AWYNLGNAYYKQGDYDEAIEYYQKALELDPNNAEAWYN | |
| NO: 69 | | LGNAYYKQGDYDEAIEYYEKALELDPENLEALQNLLNA | |
| | | MDKQG | |
| T33_dn10B | | IEEVVAEMIDILAESSKKSIEELARAADNKTTEKAVAE | |
| SEQ ID | | AIEEIARLATAAIQLIEALAKNLASEEFMARAISAIAE | |
| NO: 70 | | LAKKAIEAIYRLADNHTTDTFMARAIAAIANLAVTAIL | |
| | | AIAALASNHTTEEFMARAISAIAELAKKAIEAIYRLAD | |
| | | NHTTDKFMAAAIEAIALLATLAILAIALLASNHTTEKF | |
| | | MARAIMAIAILAAKAIEAIYRLADNHTSPTYIEKAIEA | |
| | | IEKIARKAIKAIEMLAKNITTEEYKEKAKKIIDIIRKL | |
| | | AKMAIKKLEDNRT | |
| I53_dn5A | pentamer | KYDGSKLRIGILHARWNAEIILALVLGALKRLQEFGVK | |
| SEQ ID | | RENIIIETVPGSFELPYGSKLFVEKQKRLGKPLDAIIP | |
| NO: 71 | | IGVLIKGSTMHFEYICDSTTHQLMKLNFELGIPVIFGV | |
| | | LTCLTDEQAEARAGLIEGKMHNHGEDWGAAAVEMATKF | |
| | | N | |
| I53_dn5B | trimer | EEAELAYLLGELAYKLGEYRIAIRAYRIALKRDPNNAE | |
| SEQ ID | | AWYNLGNAYYKQGRYREAIEYYQKALELDPNNAEAWYN | |
| NO: 72 | | LGNAYYERGEYEEAIEYYRKALRLDPNNADAMQNLLNA | |
| | | KMREE | |
| I53_dn5A. | pentamer | KYDGSKLRIGILHARGNAEIILALVLGALKRLQEFGVK | |
| 1 SEQ ID | | RENIIIETVPGSFELPYGSKLFVEKQKRLGKPLDAIIP | |
| NO: 73 | | IGVLIRGSTPHFDYIADSTTHQLMKLNFELGIPVIFGV | |
| | | ITADTDEQAEARAGLIEGKMHNHGEDWGAAAVEMATKF | |
| | | N | |
| I53_dn5A. | pentamer | KYDGSKLRIGILHARGNAEIILELVLGALKRLQEFGVK | |
| 2 SEQ ID | | RENIIIETVPGSFELPYGSKLFVEKQKRLGKPLDAIIP | |
| NO: 74 | | IGVLIRGSTAHFDYIADSTTHQLMKLNFELGIPVIFGV | |
| | | LTTESDEQAEERAGTKAGNHGEDWGAAAVEMATKFN | |
| I3-01 | | MKMEELFKKHKIVAVLRANSVEEAKKKALAVFLGGVHL | |
| SEQ ID | | IEITFTVPDADTVIKELSFLKEMGAIIGAGTVTSVEQC | |
| NO: 105 | | RKAVESGAEFIVSPHLDEEISQFCKEKGVFYMPGVMTP | |
| | | TELVKAMKLGHTILKLFPGEVVGPQFVKAMKGPFPNVK | |
| | | FVPTGGVNLDNVCEWFKAGVLAVGVGSALVKGTPVEVA | |
| | | EKAKAFVEKIRGCTE | |
| I3-01 | | MKIEELFKKHKIVAVLRANSVEEAKKKALAVFLGGVHL | |
| (M31) | | IEITFTVPDADTVIKELSFLKEMGAIIGAGTVTSVEQC | |
| SEQ ID | | RKAVESGAEFIVSPHLDEEISQFCKEKGVEYMPGVMTP | |
| NO: 106 | | TELVKAMKLGHTILKLFPGEVVGPQFVKAMKGPFPNVK | |
| | | FVPTGGVNLDNVCEWFKAGVLAVGVGSALVKGTPVEVA | |
| | | EKAKAFVEKIRGCTE | |
| 1WA3-ref | | MKMEELFKKHKIVAVLRANSVEEAKEKALAVFEGGVHL | |
| SEQ ID | | IEITFTVPDADTVIKELSFLKEKGAIIGAGTVTSVEQC | |
| NO: 107 | | RKAVESGAEFIVSPHLDEEISQFCKEKGVFYMPGVMTP | |
| | | TELVKAMKLGHTILKLFPGEVVGPQFVKAMKGPFPNVK | |
| | | FVPTGGVNLDNVCEWFKAGVLAVGVGSALVKGTPDEVR | |
| | | EKAKAFVEKIRGCTE | |
| 1WA3-1 | | (MK)MEELFKKHKIVAVLRANSVEEAKKKALAVFLGGV | |
| SEQ ID | | HLIEITFTVPDADTVIKELSFLKEMGAIIGAGTVTSVE | |
| NO: 108 | | QCRKAVESGAEFIVSPHLDEEISQFCKEKGVFYMPGVM | |
| | | TPTELVKAMKLGHTILKLFPGEVVGPQFVKAMKGPFPN | |
| | | VKFVPTGGVNLDNVCEWFKAGVLAVGVGSALVKGTPVE | |
| | | VAEKAKAFVEKIRGCTE | |
| 1WA3-2 | | (MK)IEELFKKHKIVAVLRANSVEEAKKKALAVFLGGV | |
| SEQ ID | | HLIEITFTVPDADTVIKELSFLKEMGAIIGAGTVTSVE | |
| NO: 109 | | QCRKAVESGAEFIVSPHLDEEISQFCKEKGVFYMPGVM | |
| | | TPTELVKAMKLGHTILKLFPGEVVGPQFVKAMKGPFPN | |
| | | VKFVPTGGVNLDNVCEWFKAGVLAVGVGSALVKGTPVE | |
| | | VAEKAKAFVEKIRGCTE | |
| 1WA3-3 | | (MK)MEELFKKHKIVAVLRANSVEEAKKKALAVFLGGV | |
| SEQ ID | | HLIEITFTVPDADTVIKELSFLKEMGAIIGAGTVTSVE | |
| NO: 110 | | QARKAVESGAEFIVSPHLDEEISQFAKEKGVFYMPGVM | |
| | | TPTELVKAMKLGHTILKLFPGEVVGPQFVKAMKGPFPN | |
| | | VKFVPTGGVNLDNVCEWFKAGVLAVGVGSALVKGTPVE | |
| | | VAEKAKAFVEKIRGCTE | |
| 1WA3-4 | | (MK)MEELFKKHKIVAVLRANSVEEAKMKALAVFVGGV | |
| SEQ ID | | HLIEITFTVPDADTVIKELSFLKELGAIIGAGTVTSVE | |
| NO: 111 | | QCRKAVESGAEFIVSPHLDEEISQFCKEKGVFYMPGVM | |
| | | TPTELVKAMKLGHTILKLFPGEVVGPQFVKAMKGPFPN | |
| | | VKFVPTGGVNLDNVCEWFKAGVLAVGVGSALVKGTIAE | |
| | | VAAKAAAFVEKIRGCTE | |
| 1WA3-5 | | (MK)MEELFKKHKIVAVLRANSVEEAKKKALAVFLGGV | |
| SEQ ID | | DLIEITFTVPDADTVIKELSFLKEMGAIIGAGTVTSVE | |
| NO: 112 | | QCRKAVESGAEFIVSPHLDEEISQFCKEKGVFYMPGVM | |
| | | TPTELVKAMKLGHTILKLFPGEVVGPQFVKAMKGPFPN | |
| | | VKFVPTGGVNLDNVCEWFKAGVQAVGVGEALNKGTPVE | |
| | | VAEKAKAFVEKIRGCTE | |
| 1WA3-6 | | (MK)MEELFKKHKIVAVLRANSVEEAKKKALAVFMGGV | |
| SEQ ID | | DLIEITFTVPDADTVIKELSFLKELGAIIGAGTVTSVE | |
| NO: 113 | | QCRKAVESGAEFIVSPHLDEEISQFCKEKGVFYMPGVM | |
| | | TPTELVKAMKLGHTILKLFPGEVVGPQFVKAMKGPFPN | |
| | | VKFVPTGGVNLDNVCEWFKAGVQAVGVGEALNKGTPAE | |
| | | VAEKAKAFVEKIRGCTE | |
| I3-01 | | (METDTLLLWVLLLWVPGSTGDYKDEK)MEELFKKHKI | |
| H35D | | VAVLRANSVEEAKKKALAVFLGGVDLIEITFTVPDADT | |
| SEQ ID | | VIKELSFLKEMGAIIGAGTVTSVEQCRKAVESGAEFIV | |
| NO: 702 | | SPHLDEEISQFCKEKGVFYMPGVMTPTELVKAMKLGHT | |
| | | ILKLFPGEVVGPQFVKAMKGPFPNVKFVPTGGVNLDNV | |
| | | CEWFKAGVLAVGVGSALVKGTPVEVAEKAKAFVEKIRG | |
| | | CTE(QKLISEEDLHHHHHH) | |
| I3-01 | | (METDTLLLWVLLLWVPGSTGDYKDEK)MEELFKKHKI | |
| K25D | | VAVLRANSVEEAKKDALAVFLGGVHLIEITFTVPDADT | |
| SEQ ID | | VIKELSFLKEMGAIIGAGTVTSVEQCRKAVESGAEFIV | |
| NO: 703 | | SPHLDEEISQFCKEKGVFYMPGVMTPTELVKAMKLGHT | |
| | | ILKLFPGEVVGPQFVKAMKGPFPNVKFVPTGGVNLDNV | |
| | | CEWFKAGVLAVGVGSALVKGTPVEVAEKAKAFVEKIRG | |
| | | CTE(QKLISEEDLHHHHHH) | |
| I3-01 | | (METDTLLLWVLLLWVPGSTGDYKDEK)MEELFKKHKI | |
| K25N | | VAVLRANSVEEAKKNALAVFLGGVHLIEITFTVPDADT | |
| SEQ ID | | VIKELSFLKEMGAIIGAGTVTSVEQCRKAVESGAEFIV | |
| NO: 704 | | SPHLDEEISQFCKEKGVFYMPGVMTPTELVKAMKLGHT | |
| | | ILKLFPGEVVGPQFVKAMKGPFPNVKFVPTGGVNLDNV | |
| | | CEWFKAGVLAVGVGSALVKGTPVEVAEKAKAFVEKIRG | |
| | | CTE(QKLISEEDLHHHHHH) | |
| I3-01 | | (METDTLLLWVLLLWVPGSTGDYKDEK)MEELFKKHKI | |
| L171Q | | VAVLRANSVEEAKKKALAVFLGGVHLIEITFTVPDADT | |
| SEQ ID | | VIKELSFLKEMGAIIGAGTVTSVEQCRKAVESGAEFIV | |
| NO: 705 | | SPHLDEEISQFCKEKGVFYMPGVMTPTELVKAMKLGHT | |
| | | ILKLFPGEVVGPQFVKAMKGPFPNVKFVPTGGVNLDNV | |
| | | CEWFKAGVQAVGVGSALVKGTPVEVAEKAKAFVEKIRG | |
| | | CTE(QKLISEEDLHHHHHH) | |
| I3-01 | | (METDTLLLWVLLLWVPGSTGDYKDEK)MEELFKKHKI | |
| L171Q/S17 | | VAVLRANSVEEAKKKALAVFLGGVHLIEITFTVPDADT | |
| 7E/V180N | | VIKELSFLKEMGAIIGAGTVTSVEQCRKAVESGAEFIV | |
| SEQ ID | | SPHLDEEISQFCKEKGVFYMPGVMTPTELVKAMKLGHT | |
| NO: 706 | | ILKLFPGEVVGPQFVKAMKGPFPNVKFVPTGGVNLDNV | |
| | | CEWFKAGVQAVGVGEALNKGTPVEVAEKAKAFVEKIRG | |
| | | CTE(QKLISEEDLHHHHHH) | |
| I3-01 | | (METDTLLLWVLLLWVPGSTGDYKDEK)MEELFKKHKI | |
| ‘secre- | | VAVLRANSVEEAKKKALAVFLGGVDLIEITFTVPDADT | |
| tion | | VIKELSFLKEMGAIIGAGTVTSVEQCRKAVESGAEFIV | |
| muta- | | SPHLDEEISQFCKEKGVFYMPGVMTPTELVKAMKLGHT | |
| tions’ | | ILKLFPGEVVGPQFVKAMKGPFPNVKFVPTGGVNLDNV | |
| (H35D/L17 | | CEWFKAGVQAVGVGEALNKGTPVEVAEKAKAFVEKIRG | |
| 1Q/S177E/ | | CTE(QKLISEEDLHHHHHH) | |
| V180N) | | | |
| SEQ ID | | | |
| NO: 707 | | | |
| I3-01 | | (METDTLLLWVLLLWVPGSTGD)MEELFKEHKIVAVLR | |
| ‘negative | | ANSVEEAKKKALAVFLGGVHLIEITFTVPDADTVIKEL | |
| interior’ | | SFLKEMGAIIGAGTVTSVEQAREAVESGAEFIVSPHLD | |
| SEQ ID | | EEISQFAKEEGVFYMPGVMTPTELVKAMKLGHTILKLF | |
| NO: 708 | | PGEVVGPQFVEAMKGPFPNVKFVPTGGVNLCNVAEWFE | |
| | | AGVLAVGVGSALVEGTPVEVAEKAKAFVEKIEGATE(Q | |
| | | KLISEEDLHHHHHH) | |
| I3-01 | | (METDTLLLWVLLLWVPGSTGD)MEELFKEHKIVAVLR | |
| ‘negative | | ANSVEEAKKKALAVFLGGVDLIEITFTVPDADTVIKEL | |
| interior | | SFLKEMGAIIGAGTVTSVEQAREAVESGAEFIVSPHLD | |
| with | | EEISQFAKEEGVFYMPGVMTPTELVKAMKLGHTILKLF | |
| secre- | | PGEVVGPQFVEAMKGPFPNVKFVPTGGVNLDNVAEWFE | |
| tion | | AGVQAVGVGEALNEGTPVEVAEKAKAFVEKIEGATE(Q | |
| muta- | | KLISEEDLHHHHHH) | |
| tions’ | | | |
| SEQ ID | | | |
| NO: 709 | | | |

Table 11 provides the amino acid sequence of a first assembly domain and second assembly domain of embodiments of the present disclosure. In each case, the pairs of sequences together form an 153 multimer with icosahedral symmetry. The right hand column in Table 11 identifies the residue numbers in each illustrative polypeptide that were identified as present at the interface of resulting assembled protein nanostructures (i.e., “identified interface residues”). As can be seen, the number of interface residues for the illustrative polypeptides of SEQ ID NO:13-46 range from 4-13. In various embodiments, a first assembly domain and second assembly domain comprise an amino acid sequence that is at least 70%, 75%, 80%, 85%, 90%, 91%, 92%, 93%, 94%, 95%, 96%, 97%, 98%, 99%, or 100% identical over its length, and identical at least at 1, 2, 3, 4, 5, 6, 7, 8, 9, 10, 11, 12, or 13 identified interface positions (depending on the number of interface residues for a given polypeptide), to the amino acid sequence of a polypeptide selected from the group consisting of SEQ ID NOs: 13-46. SEQ ID NOs: 47-63 represent other amino acid sequences of a first assembly domain and second assembly domain from embodiments of the present disclosure. In other embodiments, a first assembly domain and/or second assembly domain comprise an amino acid sequence that is at least 70%, 75%, 80%, 85%, 90%, 91%, 92%, 93%, 94%, 95%, 96%, 97%, 98%, 99%, or 100% identical over its length, and identical at least at 20%, 25%, 33%, 40%, 50%, 60%, 70%, 75%, 80%, 90%, or 100% of the identified interface positions, to the amino acid sequence of a polypeptide selected from the group consisting of SEQ ID NOs: 13-63.

As is the case with proteins in general, the polypeptides are expected to tolerate some variation in the designed sequences without disrupting subsequent assembly into protein nanostructures: particularly when such variation comprises conservative amino acid substitutions. As used herein, “conservative amino acid substitution” means that: hydrophobic amino acids (Ala, Gly, Met, Val, Ile, Leu, Thr) are substituted with other hydrophobic amino acids; hydrophobic amino acids with bulky side chains (Phe, Tyr, Trp) are substituted with other hydrophobic amino acids with bulky side chains; polar amino acids (Asp, Glu, Lys, Arg, Ser, Thr, Asn, Gly Tyr) are substituted with other polar amino acids; amino acids with positively charged side chains (Arg, His, Lys) are substituted with other amino acids with positively charged side chains; and amino acids with negatively charged side chains (Asp, Glu) are substituted with other amino acids with negatively charged side chains.

In various embodiments of the protein nanostructures of the invention, a first assembly domain and second assembly domain, or the vice versa, comprise polypeptides with the amino acid sequence selected from the following pairs, or modified versions thereof (i.e., permissible modifications as disclosed for the polypeptides of the invention: isolated polypeptides comprising an amino acid sequence that is at least 75%, at least 80%, at least 85%, at least 90%, at least 91%, at least 92%, at least 93%, at least 94%, at least 95%, at least 96%, at least 97%, at least 98%, at least 99%, at least 100% identical over its length, and/or identical at least at one identified interface position, to the amino acid sequence indicated by the SEQ ID NO:
SEQ ID NO:13 and SEQ ID NO:14 (I53-34A and I53-34B);
SEQ ID NO:15 and SEQ ID NO:16 (I53-40A and I53-40B);
SEQ ID NO:15 and SEQ ID NO:36 (I53-40A and I53-40B.1);
SEQ ID NO:35 and SEQ ID NO:16 (I53-40A.1 and I53-40B);
SEQ ID NO:47 and SEQ ID NO:48 (I53-40A genus and I53-40B genus);
SEQ ID NO:17 and SEQ ID NO:18 (I53-47A and I53-47B);
SEQ ID NO:17 and SEQ ID NO:39 (I53-47A and I53-47B.1);
SEQ ID NO:17 and SEQ ID NO:40 (I53-47A and I53-47B.1NegT2);
SEQ ID NO:37 and SEQ ID NO:18 (I53-47A.1 and I53-47B);
SEQ ID NO:37 and SEQ ID NO:39 (I53-47A.1 and I53-47B.1);
SEQ ID NO:37 and SEQ ID NO:40 (I53-47A.1 and I53-47B.1NegT2);
SEQ ID NO:38 and SEQ ID NO:18 (I53-47A.1NegT2 and I53-47B);
SEQ ID NO:38 and SEQ ID NO:39 (I53-47A.1NegT2 and I53-47B.1);
SEQ ID NO:38 and SEQ ID NO:40 (I53-47A.1NegT2 and I53-47B.1NegT2);
SEQ ID NO:49 and SEQ ID NO:50 (I53-47A genus and I53-47B genus);
SEQ ID NO:19 and SEQ ID NO:20 (I53-50A and I53-50B);
SEQ ID NO:19 and SEQ ID NO:44 (I53-50A and I53-50B.1);
SEQ ID NO:19 and SEQ ID NO:45 (I53-50A and I53-50B.1NegT2);
SEQ ID NO:19 and SEQ ID NO:46 (I53-50A and I53-50B.4PosT1);
SEQ ID NO:41 and SEQ ID NO:20 (I53-50A.1 and I53-50B);
SEQ ID NO:41 and SEQ ID NO:44 (I53-50A.1 and I53-50B.1);
SEQ ID NO:41 and SEQ ID NO:45 (I53-50A.1 and I53-50B.1NegT2);
SEQ ID NO:41 and SEQ ID NO:46 (I53-50A.1 and I53-50B.4PosT1);
SEQ ID NO:42 and SEQ ID NO:20 (I53-50A.1NegT2 and I53-50B);
SEQ ID NO:42 and SEQ ID NO:44 (I53-50A.1NegT2 and I53-50B.1);
SEQ ID NO:42 and SEQ ID NO:45 (I53-50A.1NegT2 and I53-50B.1NegT2);
SEQ ID NO:42 and SEQ ID NO:46 (I53-50A.1NegT2 and I53-50B.4PosT1);
SEQ ID NO:43 and SEQ ID NO:20 (I53-50A.1PosT1 and I53-50B);
SEQ ID NO:43 and SEQ ID NO:44 (I53-50A.1PosT1 and I53-50B.1);
SEQ ID NO:43 and SEQ ID NO:45 (I53-50A.1PosT1 and I53-50B.1NegT2);
SEQ ID NO:43 and SEQ ID NO:46 (I53-50A.1PosT1 and I53-50B.4PosT1);
SEQ ID NO:51 and SEQ ID NO:52 (I53-50A genus and I53-50B genus);
SEQ ID NO:21 and SEQ ID NO:22 (I53-51A and I53-51B);
SEQ ID NO:23 and SEQ ID NO:24 (152-03A and I52-03B);
SEQ ID NO:25 and SEQ ID NO:26 (152-32A and I52-32B);
SEQ ID NO:27 and SEQ ID NO:28 (152-33A and 152-33B)
SEQ ID NO:29 and SEQ ID NO:30 (132-06A and I32-06B);
SEQ ID NO:31 and SEQ ID NO:32 (132-19A and I32-19B);
SEQ ID NO:33 and SEQ ID NO:34 (132-28A and I32-28B);
SEQ ID NO:35 and SEQ ID NO:36 (I53-40A.1 and I53-40B.1);
SEQ ID NO:53 and SEQ ID NO:54 (T32-28A and T32-28B);
SEQ ID NO:55 and SEQ ID NO:56 (T33-09A and T33-09B);
SEQ ID NO:57 and SEQ ID NO:58 (T33-15A and T33-15B);
SEQ ID NO:59 and SEQ ID NO:60 (T33-21A and T33-21B);
SEQ ID NO:61 and SEQ ID NO:62 (T33-28A and T32-28B); and
SEQ ID NO:63 and SEQ ID NO:56 (T33-31A and T33-09B (also referred to as T33-31B)).

In some embodiments, the assembly domains are 153_dn5B (trimer, optionally linked to the antigen) and 153_dn5A or 153_dn5A.1 or 153_dn5A.2 (pentamer). I53_dn5 nanostructures are described in US 2022/0072120 A1, the contents of which are incorporated by reference. 153_dn5 variants may include one or more amino acid substitutions, such as C94A, C119A, W18G, K84R, M88P, E91D, L117I, or L120D (together “153_dn5A.1”; Ueda et al. eLife 9:e57659 (2020)) or A25E, M88A, C119T, L120E, A127E, L131T, 1132K, E133A, or a deletion of positions 135-137 (“I53_dn5A.2”; Wang et al. bioRxiv 2022.08.04.502842).

In some embodiments, the ectodomains are expressed as a fusion protein with a first assembly domain. In some embodiments, the first assembly domain and the ectodomain are joined by a linker sequence.

Non-limiting examples of designed protein complexes useful in protein nanostructures of the present disclosure include those disclosed in U.S. Pat. No. 9,630,994; Int'l Pat. Pub No. WO2018187325A1; U.S. Pat. Pub. No. 2018/0137234 A1; U.S. Pat. Pub. No. 2019/0155988 A2, each of which is incorporated herein in its entirety.

In various embodiments of the protein nanostructures of the disclosure, the assembly domains are polypeptides with the amino acid sequence selected from the following pairs, or modified versions thereof (i.e., permissible modifications as disclosed for the polypeptides of the invention: isolated polypeptides comprising an amino acid sequence that is at least 70%, 75%, 80%, 85%, 90%, 91%, 92%, 93%, 94%, 95%, 96%, 97%, 98%, 99%, or 100% identical over its length, and/or identical at least at one identified interface position, to the amino acid sequence indicated by the SEQ ID NO):
SEQ ID NO: 65 and SEQ ID NO: 66 (T33_dn2A and T33_dn2B);
SEQ ID NO: 67 and SEQ ID NO: 68 (T33_dn5A and T33_dn5B);
SEQ ID NO: 69 and SEQ ID NO: 70 (T33_dn10A and T33_dn10B); or
SEQ ID NO: 71 and SEQ ID NO: 72 (153_dn5A and 153_dn5B).

Various protein nanostructures are known in the art and described, for example in U.S. Pat. Pub. Nos. US 2015/0356240 A1; US 2016/0122392 A1, US 2018/0030429 A1, US 2019/0341124 A1, and US 2022/0072120 A1, the contents of which are incorporated by reference herein. In some embodiments, the protein nanostructure comprises, as an assembly domain, a variant of KDPG aldolase (Protein Data Bank code 1WA3) engineered to self-assemble into a protein nanostructure. In its native form, 1WA3 non-covalently assembles to form a trimer via a first interface (the trimer interface). When 20 copies of the trimer (60 monomers) are computationally docked to form a one-component icosahedral protein nanostructure, sets of five monomers of 1WA3 contact one another via a second interface (the pentamer interface). By introducing amino acid substitutions, the pentamer interface may be stabilized such that the protein nanostructure will spontaneously self-assemble, e.g., within the expressing cell or when isolated trimers (or monomers) are mixed under suitable conditions.

In some embodiments, the pentamer interface comprises 1, 2, 3, 4 or more interface residues, such as residues in positions 33, 61, 187, and 190 numbered according to SEQ ID NO: 107. In some embodiments, the assembly domain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 107. In some embodiments, the assembly domain comprises amino acid substitutions at 1, 2, 3, 4 of positions 33, 61, 187, and 190 compared to SEQ ID NO: 107. In some embodiments, a plurality of the amino acid substitutions are substitutions of a polar residue for a non-poplar residue (e.g., A, L, I, M, V, F, or W). In some embodiments, some or all of the amino acid substitutions are substitutions of a polar residue for a small, non-polar residue (e.g., A, L, I, M, or V). In some embodiments, the protein nanostructure comprises amino acid substitutions E33L or E33V; K61L or K61M; D187A or D187V; and/or R190A. In some embodiments, the protein nanostructure comprises amino acid substitutions E33L, K61M, D187V, and R190A. In some embodiments, the protein nanostructure comprises amino acid substitutions E33V, K61L, D187A, and R190A. In some embodiments, the assembly domain comprises an amino acid substitution to negate the enzymatic activity of the assembly domain (e.g., K129A). In embodiments, the assembly domain may comprise further amino acid substitutions (e.g., MI3; E56M or E56K; P186I; E191A; and/or K194A). In some embodiments, the assembly domain comprises amino acid substitutions that remove cysteine residues. In some embodiments, the assembly domain comprises C76A and/or C100A substitutions.

In some embodiments, the polypeptide comprises a heterologous multimerization domain. In some embodiments, the multimerization domain is a trimerization domain. In some embodiments, the multimerization domain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64). In some embodiments, the ectodomain comprises the amino acid substitutions S155C, S290C, S190F, and V207L.

In some embodiments, the trimeric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of the sequences listed in Table 19 or to any one of the sequences listed in Table 19 without the underlined and/or bold/italicized polypeptide sequences.

### Ferritin-Based Nanostructures

In some embodiments, the assembly domain is a ferritin polypeptide. In some embodiments, the assembly domain of a ferritin protein nanostructure comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of the following sequences:

(SEQ ID NO: 114)
MLSKDIIKLLNEQVNKEMNSSNLYMSMSSWCYTHSLDGAGLFLFDHAAEE
YEHAKKLIIFLNENNVPVQLTSISAPEHKFEGLTQIFQKAYEHEQHISES
INNIVDHAIKSKDHATFNFLQWYVAEQHEEEVLFKDILDKVELIGNENHG
LYLADQYVKGIAKSRKS.
(SEQ ID NO: 115)
MLKPEMIEKLNEQMNLELYSSLLYQQMSAWCSYHTFEGAAAFLRRHAQEE
MTHMQRLFDYLTDTGNLPRINTVESPFAEYSSLDELFQETYKHEQLITQK
INELAHAAMTNQDYPTFNFLQWYVSEQHEEEKLFKSIIDKLSLAGKSGEG
LYFIDKELSTLDAQN.
(SEQ ID NO: 116)
NFHQDCEAGLNRTVNLKFHSSYVYLSMASYFNRDDVALSNFAKFFRERSE
EEKEHAEKLIEYQNQRGGRVFLQSVEKPERDDWANGLEALQTALKLQKSV
NQALLDLHAVAADKSDPHMTDFLESPYLSESVETIKKLGDHITSLKKLWS
SHPGMAEYLFNKHTLG.
(SEQ ID NO: 117)
QFSKDIEKLLNEQVNKEMQSSNLYMSMSSWCYTHSLDGAGLFLFDHAAEE
YEHAKKLIIFLNENNVPVQLTSISAPEHKFEGLTQIFQKAYEHEQHISES
INNIVDHAIKSKDHATFNFLQWYVAEQHEEEVLFKDILDKIELIGNENHG
LYLADQYVKGIAKSRKSGS.
(SEQ ID NO: 118)
SGESQVRQNFKPEMEEKLNEQMNLELYSSLLYQQMSAWCSYHTFEGAAAF
LRRHAQEEMTHMQRLFDYLTDTGNLPRINTVESPFAEYSSLDELFQETYK
HEQLITQKINELAHAAMTNQDYPTFNFLQWYVSEQHEEEKLFKSIIDKLS
LAGKSGEGLYFIDKELSTLDGS.

In some embodiments, the C-terminal helix-forming segment links antigen with any nanoparticle known in the art-including but not limited to HPV particle (with SpyCatcher), or Ferritin.

### Other Nanostructures or Nanoparticles

In some embodiments, the ecotdomains described herein are displayed on any nanostructure or nanoparticle known in the art. Illustrative nanostructures and nanoparticles include, but are not limited to Human papillomavirus (HPV) virus-like particles (VLPs), Chikungunya VLPs, AP205 capsid protein VLPs, phage VLPs (e.g., bacteriophage). Display on these and other platforms may be performed by creating a fusion protein of the ectodomain to a relevant protein of the system, by bioconjugate chemistry (e.g., SpyCatcher), or other means known in the art. The protein nanostructure may be a lumazine synthase nanoparticle as described, e.g., in Geng et al. PLOS Pathog. 17 (9):e1009897 (2021). The protein nanostructure may be a ferritin nanoparticle as described, e.g., in Joyce et al. bioRxiv 2021.05.09.443331 and in U.S. Pat. Pub. No. US 2019/0330279 A1.

In another aspect, the disclosure provides a recombinant polypeptide for use in displaying a molecule such as an antigen, comprising an alpha-helical segment and a multimerization domain, wherein the alpha-helical segment comprises one or more amino acid substitutions selected such that the segment forms a stable alpha-helical homotrimer. In some embodiments, the alpha-helical segment has improved hydrophobic packing. In some embodiments, the alpha-helical segment comprises between about 7 and about 31 residues. In some embodiments, the amino acid substitutions comprise polar, charged and/or hydrophobic amino acids.

In some embodiments, the alpha-helical segment comprises a polypeptide sequence according to any one of LXXTIXXLLXIXXXLXXXL (SEQ ID NO: 566), LVXTXKXLXDLIXXLXXLLXKLXX (SEQ ID NO: 567), LNKVKKXVXXLXXXVXXLEKXLX (SEQ ID NO: 568), EKIXXAIKKAXKL (SEQ ID NO: 569), EXIXKAIKXLXXXXX (SEQ ID NO: 570), XKXXEXXXXVXXXXXXXXX (SEQ ID NO: 571), XXLKKAAXIXKKXLKXX (SEQ ID NO: 572).

In some embodiments, the alpha-helical segment comprises a polypeptide sequence according to any one of the consensus sequences in Table 24.

In some embodiments, the alpha-helical segment comprises a polypeptide sequence according to a) L X₂ X₂ T I X₂ X₂ L L X₂ I [V/I] X₂ X₂ L [I/L] X₂ X₂ L (SEQ ID NO: 573), b) L V [A/T] T X₂ K X₂ L X₂ D L I X₂ X₂ L [K/E] X₂ L L X₂ K L X₂ X₂ (SEQ ID NO: 574), or c) L N K V K K X₂ V X₂ X₂ L X₂ X₂ X₂ V X₂ X₂ L E K X₂ L X₂ (SEQ ID NO: 575), wherein X₂ is polar and charged residues selected from S, T, N, Q, E, D, R, K, and H, preferably wild type amino acid.

In some embodiments, the alpha-helical segment comprises a polypeptide sequence according a) E K I X₂ X₂ A I K K A X₂ KL (SEQ ID NO: 576), b) E X₂ I X₂ K A I K X₂ L [L/X₂] X₂ X₂ [X₁/X₂] X₂ (SEQ ID NO: 577), and c) X₂ K [X₁/T] [L/E] E [T/A] X₁ X₂ [I/X₂] V X₂ X₂ [X₁/X₂] [X₁/X₂] X₂ X₂ X₁ X₂ X₂ (SEQ ID NO: 578), or d) X₂ X₂ L K K A A X₂ I X₁ K K X₁ L K X₂ X₂ (SEQ ID NO: 579), wherein X₁ is apolar residues selected from A, I, L, and M, wherein X₂ is polar and charged residues selected from S, T, N, Q, E, D, R, K, and H, preferably wild type amino acid.

In some embodiments, the alpha-helical segment comprises a polypeptide sequence listed in Table 25A or Table 25B or a polypeptide sequence having between 1 and 5 amino acid substitutions thereto. In some embodiments, the alpha-helical segment comprises the polypeptide sequence NQSREIIRAINIVRKIASEK (SEQ ID NO: 10), NQSALWLEAAKYVKQAREKS (SEQ ID NO: 11), NQSAKNAEAAKIAEETKRKD (SEQ ID NO: 12), or NQSRETAKAVSAVK (SEQ ID NO: 75), or a polypeptide sequence having between 1 and 5 amino acid substitutions thereto.

In some embodiments, the multimerization domain is I53-50A or a variant thereof. In some embodiments, the multimerization domain comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to I53-50A (SEQ ID NO: 19) or I53-50A ΔCys (SEQ ID NO: 64). In some embodiments, the polypeptide comprises an N-terminal fusion of the alpha-helical segment to the multimerization domain via a peptide bond or polypeptide linker. In some embodiments, the polypeptide comprises, N-terminal to the alpha-helical segment, an antigen polypeptide.

In another aspect, the disclosure provides a polypeptide comprising an alpha-helical segment, comprising a polypeptide sequence listed in Table 25A or Table 25B, or a polypeptide sequence having between 1 and 5 amino acid substitutions thereto. In another aspect, the disclosure provides a protein nanostructure comprising a trimeric component comprising a polypeptide described herein. In some embodiments, the nanostructure is a two-component nanostructure comprising a first, trimeric component and a second, pentameric component. In some embodiments, the nanostructure is a two-component nanostructure comprising a second pentameric component, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to SEQ ID NO: 20 or 71.

### IV. Polynucleotides

In another aspect, the present disclosure provides polynucleotides encoding any of the polypeptides, complex, components, nanostructures, or other compositions of the disclosure. The polynucleotides sequences may comprise RNA or DNA. As used herein, “polynucleotides” are those that have been removed from their normal surrounding polynucleotides sequences in the genome or in cDNA sequences. Such polynucleotides sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the disclosure.

### V. Delivery Vehicles

In some embodiments, polynucleotides (e.g., mRNA) encoding protein nanostructures including a component comprising a viral protein monomer of a trimeric viral antigen are formulated in a delivery vehicle. In some embodiments, the delivery vehicle is a non-viral vector. In some embodiments, the delivery vehicle is a lipid nanoparticle (LNP). In some embodiments, the delivery vehicle is a liposome. In some embodiments, the delivery vehicle is a polymeric-non-viral vector, such as spermine, Polyethylenimine, chitosan, or polyurethane. In some embodiments, the delivery vehicle is a polymer delivery system, such as poly-amido-amine (PAA), poly-beta aminoesters (PBAEs) or polyethylenimine (PEI). In some embodiments, the delivery vehicle is a ferritin nanoparticle. In some embodiments, the delivery vehicle is an encapsulin.

In some embodiments, polynucleotides (e.g., mRNA) encoding protein nanostructures including a component comprising a viral protein monomer of a trimeric viral antigen are formulated in a nanoparticle. In some embodiments, the nanoparticle is a lipid nanoparticle (LNP). In some embodiments, the polynucleotides are formulated in a lipid-polycation complex, referred to as a cationic LNP. As a non-limiting example, the polycation may include a cationic peptide or a polypeptide such as, but not limited to, polylysine, polyornithine and/or polyarginine. In some embodiments, the polynucleotides are formulated in a LNP that includes a non-cationic lipid such as, but not limited to, cholesterol or dioleoyl phosphatidylethanolamine (DOPE).

In various embodiments, the lipid nanoparticles have a mean diameter from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the LNPs are substantially non-toxic. In certain embodiments, polynucleotides, when present in the LNPs, are resistant in aqueous solution to degradation with a nuclease. Lipids and LNPs comprising polynucleotides and their method of preparation are described in, e.g., U.S. Pat. Nos. 8,569,256, 5,965,542 and U.S. Patent Publication Nos. 2021/0323914, 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/004143, WO 2017/075531, WO 2015/199952, WO 2014/008334, WO 2013/086373, WO 2013/086322, WO 2013/016058, WO 2013/086373, WO2011/141705, WO 2017/049245, WO 2010/144740, WO/2017/075531, and WO 2001/07548, the contents of which are incorporated by reference herein.

Further exemplary lipids and LNPs and their manufacture are known in the art—for example in U.S. Pat. Pub. No. U.S. 2012/0276209, Semple et al., 2010, Nat Biotechnol., 28 (2): 172-176; Akinc et al., 2010, Mol Ther., 18 (7): 1357-1364; Basha et al., 2011, Mol Ther, 19 (12): 2186-2200; Leung et al., 2012, J Phys Chem C Nanomater Interfaces, 116 (34): 18440-18450; Lee et al., 2012, Int J Cancer., 131 (5): E781-90; Belliveau et al., 2012, Mol Ther nucleic Acids, 1: e37; Jayaraman et al., 2012, Angew Chem Int Ed Engl., 51 (34): 8529-8533; Mui et al., 2013, Mol Ther Nucleic Acids. 2, e139; Maier et al., 2013, Mol Ther., 21 (8): 1570-1578; and Tam et al., 2013, Nanomedicine, 9 (5): 665-74, each of which are incorporated by reference herein. Lipids and their manufacture can be found, for example, in U.S. Pat. Pub. Nos. 2015/0376115 and 2016/0376224, the contents of which are incorporated by reference herein.

### VI. Pharmaceutical Compositions

The disclosure also provides pharmaceutical compositions. Such pharmaceutical compositions can be used for generating an immune response against an infectious disease in a subject. The pharmaceutical compositions of the disclosure may include a pharmaceutically acceptable carrier. A thorough discussion of such carriers is available in Chapter 30 of Remington: The Science and Practice of Pharmacy (23^{rd} ed., 2021).

In some embodiments, the pharmaceutical composition can also include excipients and/or additives. Examples of these are surfactants, stabilizers, complexing agents, antioxidants, or preservatives which prolong the duration of use of the finished pharmaceutical formulation, flavorings, vitamins, or other additives known in the art. Complexing agents include, but are not limited to, ethylenediaminetetraacetic acid (EDTA) or a salt thereof, such as the disodium salt, citric acid, nitrilotriacetic acid and the salts thereof. In some embodiments, preservatives include, but are not limited to, those that protect the solution from contamination with pathogenic particles, including benzalkonium chloride or benzoic acid, or benzoates such as sodium benzoate. Antioxidants include, but are not limited to, vitamins, provitamins, ascorbic acid, vitamin E, salts or esters thereof.

Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethyl cellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

In some embodiments, one or more tonicity agents may be added to provide the desired ionic strength. Tonicity agents for use herein include those which display no or only negligible pharmacological activity after administration. Both inorganic and organic tonicity adjusting agents may be used.

In another aspect, the disclosure provides a pharmaceutical composition comprising a polypeptide, a protein complex, or a nanostructure disclosed herein.

### VII. Vaccines

In another aspect, the disclosure provides a vaccine comprising a polypeptide, a protein complex, or a nanostructure as disclosed herein. In another aspect, the disclosure provides a vaccine comprising a polypeptide or a nanostructure described herein.

In some embodiments, the vaccine comprises an adjuvant.

In some embodiments, the pharmaceutical composition provided herein is administered as a RSV vaccine, for example, an RSV/A vaccine, and RSV/B vaccine, or a bivalent RSV A/B vaccine. In some embodiments, the pharmaceutical composition provided herein is administered as a hMPV vaccine. In some embodiments, the pharmaceutical composition provided herein is administered as a hMPV/A vaccine. In some embodiments, the pharmaceutical composition provided herein is administered as a hMPV/B vaccine. In some embodiments, the pharmaceutical composition provided herein is administered as a hMPV/A and hMPV/B bivalent vaccine. In some embodiments, the pharmaceutical composition provided herein is administered as a hMPV/A and RSV bivalent vaccine In some embodiments, the pharmaceutical composition provided herein is administered as a PIV3 vaccine. In some embodiments, the pharmaceutical composition provided herein is administered as a PIV5 vaccine. In some embodiments, the pharmaceutical composition provided herein is administered as a SARS-COV-2 vaccine. In some embodiments, the pharmaceutical composition provided herein is administered as a Nipah vaccine. In some embodiments, the pharmaceutical composition provided herein is administered as a bivalent RSV/hMPV vaccine.

### Adjuvants

Adjuvants or immune potentiators may also be administered with or in combination with a lipid nanoparticle composition. Advantages of adjuvants include, but are not limited to, the enhancement of the immunogenicity of antigens, modification of the nature of the immune response, the reduction of the antigen amount needed for a successful immunization, the reduction of the frequency of booster immunizations needed and an improved immune response in elderly and immunocompromised vaccines. These may be co-administered by any route, e.g., intramuscular, subcutaneous, intravenous, or intradermal injections.

Adjuvants may include, but are not limited to, a natural or a synthetic adjuvant. Adjuvants may be organic or inorganic.

Adjuvants may be selected from any of the classes (1) mineral salts, e.g., aluminum hydroxide and aluminum or calcium phosphate gels; (2) emulsions including: oil emulsions and surfactant based formulations, e.g., microfluidised detergent stabilized oil-in-water emulsion, purified saponin, oil-in-water emulsion, stabilized water-in-oil emulsion; (3) particulate adjuvants, e.g., virosomes (unilamellar liposomal vehicles incorporating influenza hemagglutinin), structured complex of saponins and lipids, polylactide co-glycolide (PLG); (4) microbial derivatives; (5) endogenous human immunomodulators; (6) inert vehicles, such as gold particles; (7) microorganism derived adjuvants; (8) tensoactive compounds; (9) carbohydrates; or combinations thereof.

Adjuvants for nucleic acid vaccines (DNA) have been disclosed in, for example, Kobiyama, et al., Vaccines, 2013, 1(3), 278-292, the contents of which are incorporated herein by reference in their entirety. Any of the adjuvants disclosed by Kobiyama et al., may be used in the vaccines as described herein.

Other adjuvants which may be utilized include any of those listed on the web-based vaccine adjuvant database, on the World Wide Web at violinet.org/vaxjo/and described in for example Sayers, et al., J. Biomedicine and Biotechnology, volume 2012 (2012), Article ID 831486, 13 pages, the contents of which are incorporated herein by reference in their entirety.

Specific adjuvants may include cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derived P40 Vaccine Adjuvant, MPL™ Adjuvant, AS04, AS02, AS01E, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, ADJUMER™, Algal Glucan, Bay R1005, Theramide®, Stearyl Tyrosine, Specol, Algammulin, AVRIDINE®, Calcium Phosphate Gel, CTA1-DD gene fusion protein, DOC/Alum Complex, Gamma Inulin, Gerbu Adjuvant, GM-CSF, GMDP, Recombinant hIFN-gamma/Interferon-g, Interleukin-1ß, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E112K of Cholera Toxin mCT-E112K, and/or Matrix-S.

In some embodiments, the adjuvant comprises squalene. In some embodiments, the adjuvant comprises aluminum hydroxide. In some embodiments, the adjuvant comprises AS01_{E}.

### VIII. Methods of Use

In another aspect, the disclosure provides methods of administration for the composition, the pharmaceutical composition, or the vaccine described herein.

In another aspect, the disclosure provides a method of vaccinating a subject, comprising administering to the subject a composition described herein. In another aspect, the disclosure provides a method of generating an immune response in a subject, comprising administering to the subject a composition described herein. In another aspect, the disclosure provides a method of treating or preventing disease including, but not limited to, RSV, hMPV, PIV3, PIV5, Nipah and/or SARS-COV-2 in a subject, comprising administering to the subject a composition described herein. In another aspect, the disclosure provides a composition of the disclosure for use in vaccinating, generating an immune response, or treating or preventing disease including, but not limited to, RSV, hMPV, PIV3, PIV5, Nipah and/or SARS-COV-2. In another aspect, the disclosure provides a composition, method, or use as described herein. In another aspect, the disclosure provides a method of treating or preventing coronavirus disease in a subject, comprising administering to the subject a composition described herein. In another aspect, the disclosure provides a composition of the disclosure for use in vaccinating, generating an immune response, or treating or preventing coronavirus disease. In another aspect, the disclosure provides a composition, method, or use as described herein.

In another aspect, the disclosure provides a method of vaccinating a subject, comprising administering to the subject a composition described herein. In another aspect, the disclosure provides a method of generating an immune response or treating or preventing a viral infection in a subject, comprising administering to the subject a polypeptide or a nanostructure described herein. In another aspect, the disclosure provides a method of making a polypeptide or a nanostructure described herein, comprising culturing host cells modified to express one or more polypeptides as described herein.

In some embodiments, the method comprises administering the vaccine described herein. In some embodiments, the subject is immunized against infection to RSV, hMPV, PIV3, PIV5, Nipah and/or SARS-COV-2 S. In some embodiments, the subject is immunized against infection by coronavirus. In some embodiments, the vaccine is administered by subcutaneous injection. In some embodiments, the vaccine is administered by intramuscular injection. In some embodiments, the vaccine is administered by intradermal injection. In some embodiments, the vaccine is administered intranasally. In one aspect, the disclosure provides a pre-filled syringe comprising the vaccine described herein. In one aspect, the disclosure provides a kit comprising the vaccine described herein or the pre-filled syringe described herein. In one aspect, the disclosure provides a kit comprising the vaccine described herein or the lysophilized vaccine described herein

In some embodiments, the unit dose of the pharmaceutical composition comprises about 0.5 μg to about 1 μg, about 20 μg to about 25 μg, about 25 μg to about 50 μg, about 50 μg to about 70 μg, about 70 μg to about 75 μg, about 75 μg to about 100 μg, about 100 μg to about 125 μg, about 100 μg to about 150 μg, about 125 μg to about 150 μg, about 125 μg to about 175 μg, about 150 μg to about 175 μg, about 175 μg to about 200 μg, about 200 μg to about 250 μg, about 225 μg to about 300 μg, about 250 μg to about 300 μg, or about 250 μg to about 350 μg of the protein nanostructures.

In some embodiments, the subject is at risk of disease including, but not limited to, RSV, hMPV, PIV3, PIV5, Nipah and/or SARS-COV-2. In some embodiments, the subject is at risk of hMPV disease. In some embodiments, the subject is at risk of PIV3 disease. In some embodiments, the subject is at risk of PIV5 disease. In some embodiments, the subject is at risk of coronavirus disease. In some embodiments, the subject is an adult of over 60 years of age. In some embodiments, the subject is a healthy adult of 18-45 years of age. In some embodiments, the subject is a pregnant women between week 32 and week 36 of pregnancy. In some embodiments, the subject is a pregnant women between week 30 and week 38 of pregnancy. In some embodiments, the subject is a pregnant women between week 28 and week 38 of pregnancy.

In another aspect, the disclosure provides a method of vaccinating a subject, comprising administering to the subject a composition disclosed herein. In another aspect, the disclosure provides a method of generating an immune response in a subject, comprising administering to the subject a composition disclosed herein. In another aspect, the disclosure provides a method of treating or preventing a viral infection in a subject, comprising administering to the subject a composition disclosed herein. In another aspect, the disclosure provides a composition for use in vaccinating, generating an immune response, or treating or preventing any viral infectious disease disclosed herein. In another aspect, the disclosure provides a composition, method, or use as described herein. In another aspect, the disclosure provides a method of making a composition, comprising culturing host cells modified to express one or more polypeptides as described herein.

Any aspect or embodiment described herein can be combined with any other aspect or embodiment as disclosed herein

### EXAMPLES

The following Examples are merely illustrative and are not intended to limit the scope or content of the invention in any way.

### Example 1. Remodeling the C-Terminus of RSV F Protein

This Example describes remodeling the C terminus of the RSV F protein to create a stable helix-forming segment.

RSV F protein, like other class I viral membrane fusion protein, forms a trimer with two primary conformations (prefusion and postfusion). The C terminus of the ectodomain, adjacent to the transmembrane domain, is believed to form a helical bundle in the context of the native protein. Structures of the prefusion F protein generally model the C terminus as alpha-helical, with structured density ending at about residue 510 or 512 (e.g., PDB 5C6B and 5UDD, respectively). The native sequence after residue 513 is often replaced with a four-residue linker (SAIG) and the trimeric FoldOn domain. The predicted transmembrane domain begins at residue 527. The sequence of a native RSV/B F protein sequence (GenBank: WDV37446.1) is shown here with the transmembrane domain bold/underlined:

(SEQ ID NO: 1)

| | |
|---|---|
| 1 | MELLIHRSSA IFLTLAINAL YLTSSQNITE EFYQSTCSAV |
| 41 | SRGYLSALRT GWYTSVITIE LSNIKETKCN GTDTKVKLIK |
| 81 | QELDKYKNAV TELQLLMQNT PAVNNRARRE APQYMNYTIN |
| 121 | TTKNLNVSIS KKRKRRFLGF LLGVGSAIAS GIAVSKVLHL |
| 161 | EGEVNKIKNA LQLTNKAVVS LSNGVSVLTS RVLDLKNYIN |
| 201 | NQLLPMVNRQ SCRISNIETV IEFQQKNSRL LEITREFSVN |
| 241 | AGVTTPLSTY MLTNSELLSL INDMPITNDQ KKLMSSNVQI |
| 281 | VRQQSYSIMS IIKEEVLAYV VQLPIYGVID TPCWKLHTSP |
| 321 | LCTTNIKEGS NICLTRTDRG WYCDNAGSVS FFPQADTCKV |
| 361 | QSNRVFCDTM NSLTLPSEVS LCNTDIFNSK YDCKIMTSKT |
| 401 | DISSSVITSL GAIVSCYGKT KCTASNKNRG IIKTFSNGCD |
| 441 | YVSNKGVDTV SVGNTLYYVN KLEGKNLYVK GEPIINYYDP |
| 481 | LVFPSDEFDA SISQVNEKIN QSLAFIRRSD ELLHNVNTGK |
| 521 | STTNIMITAI TIVIIVVLLS LIAIGLLLYC KAKNTPVTLS |
| 561 | KDQLSGINNI AFSK |

We hypothesized that the poor structural resolution of the C terminus of the ectodomain reflects imperfect hydrophobic packing of the helical bundle in the native protein when it is expressed recombinantly. We developed a pipeline to remodel the C terminus of the ectodomain to generate improved antigens for use in vaccines. Our method structurally remodels the segment (corresponding to about residue 500 and about residue 530 relative to native sequence) into a more structurally stable helical bundle by substituting residues (e.g., to generate new non-covalent interactions, prevent clashing of residues, or adjust the polypeptide backbone), as well as preserve or enhance polar exposed surfaces, and thereby decrease the free energy of self-association of the protomers (as predicted ddG and measuring thermal denaturation temperature). The remodeling pipeline included manual selection of sequences predicted to form structures capable of serving as adaptors to connect the C terminus of the ectodomain to a trimerization domain, such as an I53-50A multimerization domain. Manual selection was performed based on a combination of polypeptide sequence diversity and computational metrics, which included geometry design space, hydrophobic core packages, termini availability, and lack of obvious errors in conformation (i.e., solvent exposed tryptophans).

Structural models from the Protein Data Bank (PDB) were prepared for design by symmetrization, removal of hetero-atoms, renumbering, relaxing, and marking of glycosylation sites. Rosetta blueprint files were written to generate a gradient between the native structure and the remodeled domain. Generally, the blueprint included a two-residue native sequence that is remodeled using helical constraints; followed by one or more existing helical residues that contribute to hydrophobic contacts at the C-terminus, which are allowed to design; followed by an alpha-helical segment ranging in length from approximately 1-10 amino acids in length, determined empirically by the designer. For example, to remodel this sequence:

(SEQ ID NO: 710)
481 LVFPSDEFDA SISQVNEKIN QSLAFIRRSD ELLHNVNTG

a blueprint may be generated were the amino acid residue is set to match the native sequence (A), to start with native sequence but allow substitutions (A), to newly modeled as any amino acid (X) (top line), while the three-dimensional structure of the polypeptide is set to either match the native structure (.) or to be constrained to be helical (H):

(SEQ ID NO: 711)

| | |
|---|---|
| 481 | LVFPSDEFDA SISQVNEKIN QSLAFIRRSX XXXXXXXXX |

(SEQ ID NO: 712)

.......... .......... HHHHHHHHHH HHHHHHHHH

Using this or similar blueprints, designs were generated with Rosetta Remodel. Remodel results were filtered by ddG calculations from the design model directly, and manually reverted to remove any introduced glycosylation sites, exposed hydrophobic residues, or buried polar residues. The resulting models were relaxed and then ddG's were again calculated.

Alternatively, remodeling was performed using RFdiffusion. Relaxed structures used as input for remodel were also used as input for RFdiffusion, except that only the C-terminal helices were used as input for diffusion. This protocol significantly reduces computational time. Positions with WT sequence identities from Rosetta Remodel were likewise preserved for diffusion. Unlike with Rosetta Remodel all structural data of subsequent residues was ignored. This is a limitation of RFdiffusion, not a scientific constraint placed on the design problem. Intra-protomer and inter-protomer weights were both set to 1, with a quadratic guide decay and a guide scale of 2. C-terminal lengths varied between 12 and 31 residues depending on the source virus, and 15 remodeled helices were generated for each length. Sequences were generated using Protein MPNN, with a sampling temperature of 0.4 and a negative bias against C (−10), and F, G, P, W, and Y (−1). 100 sequences were generated for each remodeled domain. The top 2% based on MPNN sample score were selected for computational characterization.

Designs were analyzed based on the following criteria: 1) ColabFold validates the design performed with Rossetta by predicting ordered terminal helix consistent with design model (assuming ColabFold method can provide reliable results for a particular fusion protein); 2) decrease in ddG by 5-15 Rosetta Energy Unit (REU) and 3); design has a well-packed hydrophobic core without extraneous elements (i.e., helical segments with no interprotomer hydrophobic packing). To calculate ddG, two models are generated, one in which all protomers are correctly in contact as trimers and one in which the protomers are moved distant from each other. Sidechains in both models are repacked and minimized, and then both models are scored. The ddG is the difference in the scores, as in (Distant state)-(Trimeric state).

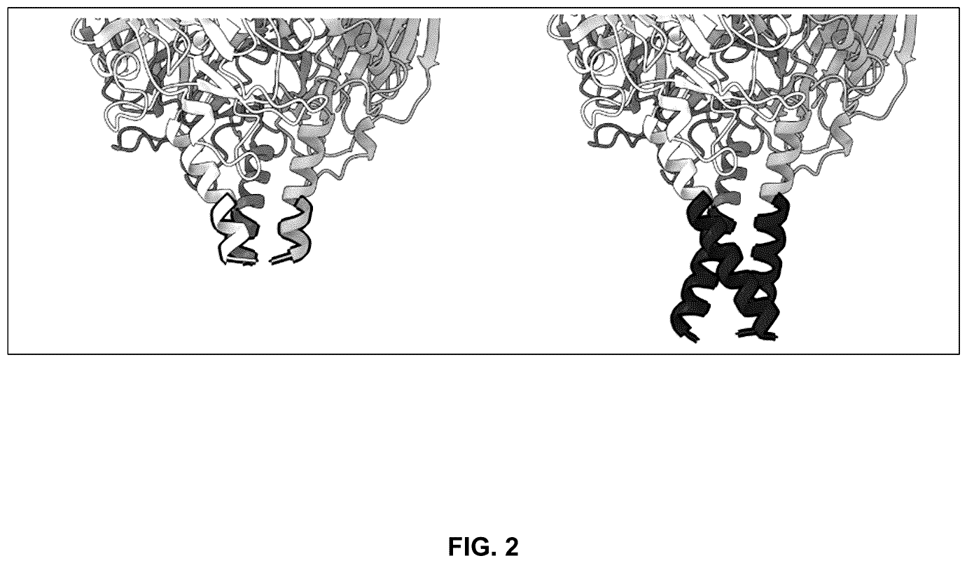

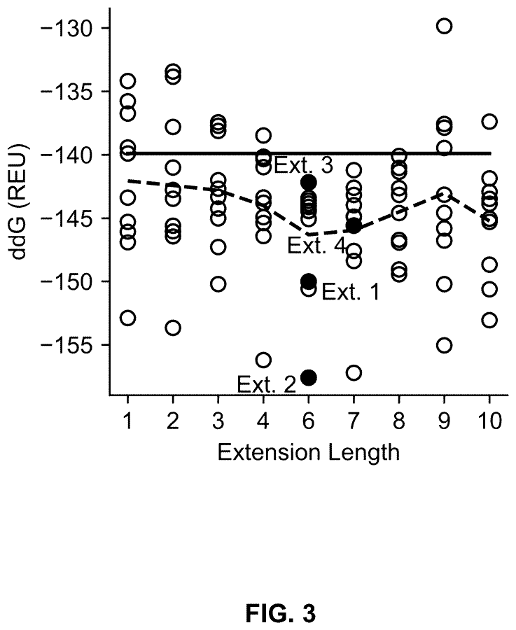

FIG. 2 shows a structural model of a representative experimental model of the RSV F protein (left) compared to the predicted structure of a representative design (right), provided from PDB 4MMU. The optimal length for the remodeled C terminus was determined by plotting average ddG against the length of the C-terminal helix, as shown in FIG. 3. When using Rosetta Remodel, the average ddG will decrease until an optimum length is achieved, at which point the ddG will tend to stay the same or increase again. This may be because Remodel can struggle when building larger segments due to increasing degrees of freedom. Ideal linker lengths are those near the minimum ddG. In this case, it was determined that an optimal C-terminal helix would terminate at about position 519. It was observed empirically that a ddG was minimized when the helical segment extended about 6 residues past the native position 513 (i.e., to position 519).

Computational modeling (with Rosetta Remodel) of the RSV/B protein was used to generate artificial polypeptide sequences, each predicted to form a stable alpha helix, shown in Table 12. Residues 500-502 of the native RSV F protein are included as NQS. Residues Q501 and S502 were remodeled with helical constraints while preserving the native sequence identities. This optimizes the helical backbone of these residues with side chains represented as centroids and then repacks the side chains in all-atom mode. Residues 503-509 were remodeled with helical constraints and without sequence constraints. The helical backbone is first optimized with side chains represented as centroids, and the side chains are designed in all-atom mode. As a result there is some bias towards the native sequence. Six to 14 additional amino acids were added with helical constraints. Side chains are represented as valine centroids during backbone sampling, then the sequence is sampled in all-atom mode. All backbone sampling of these elements in centroid mode is performed simultaneously and sequence design in all-atom mode is likewise performed simultaneously. Designs were manually refined to remove exposed hydrophobic residues or buried polar residues with identities preferentially selected from the nearest residue in the WT sequence or rationally where the WT residue was suboptimal.

The I53-50A molecule is well-suited for genetic fusion to many trimeric antigens, and features symmetric N-termini that are approximately 5 nm apart. Due to the remodeled C-terminus of the C-Term 1 design being more distanced laterally from the symmetric axis of the antigen (FIG. 2), it appeared possible that this modification could minimize strain in genetic fusions to I53-50A relative to commonly-studied antigen fragments that end at residue 513. Four sequences were selected for experimental testing (Table 12) as genetic fusions to a version of I53-50A (I53-50Aδcys), with antigens also containing DS-Cav1 mutations.

TABLE 12
Illustrative C-terminal helix-forming segments

| | | Remodeled | |
|---|---|---|---|
| Name | Sequence | Length | SEQ ID NO: |
| C-Term 1 | NQSREIIRAINIVRKIASEK | 17 | 10 |
| C-Term 2 | NQSALWLEAAKYVKQAREKS | 17 | 11 |
| C-Term 3 | NQSAKNAEAAKIAEETKRKD | 17 | 12 |
| C-Term 4 | NQSRETAKAVSAVK | 11 | 75 |
| C-Term 5 | NQSALLLEAAKYVKKAREKS | 17 | 119 |
| C-Term 6 | NQSRKLLEAAEEMEKMLKTS | 17 | 120 |
| C-Term 7 | NQSRKMLEAVEHAKKLKKES | 17 | 121 |
| C-Term 8 | NQSRKMLEAVEKAKKLDKES | 17 | 122 |
| C-Term 9 | NQSAKTEEAYQRTIKTQQKL | 17 | 123 |
| C-Term 10 | NQSRDLDTAAKQVKEMLKEKS | 18 | 124 |
| C-Term 11 | NQSRETEKTIRQVQEILKKWS | 18 | 125 |
| C-Term 12 | NQSREVKEAIKIIKKILKKQS | 18 | 126 |
| C-Term 13 | NQSREIKDAIKKAKEFIKTIK | 18 | 127 |
| C-Term 14 | NQSREIETAIKKAKEFIKTIK | 18 | 128 |
| C-Term 15 | NQSRKATETIKKFEESEKS | 16 | 129 |
| C-Term 16 | NQSRDTIKVAIIVKELYKKIS | 18 | 130 |
| C-Term 17 | NQSRKTLETIEWVKKVIKKQRS | 19 | 131 |
| C-Term 18 | NQSRKTLETIEWVEKVIKKQRS | 19 | 132 |
| C-Term 19 | NQSRKWNESSKKVQEQDS | 15 | 133 |
| C-Term 20 | NQSRKTEKAIRLVLKWLKES | 17 | 134 |
| C-Term 21 | NQSRDTLKAIEQTKRYLEELKKS | 20 | 135 |
| C-Term 22 | NQSRSWDIAAKFVKTVLSNQS | 18 | 136 |
| C-Term 23 | NQSRKTLEATEIAKKLAEDRS | 18 | 137 |
| C-Term 24 | NQSLEILKAAKEAKKLIEDLRRS | 20 | 138 |
| C-Term 25 | NQSKELLDAAKAVKKMLEKEKSS | 20 | 139 |
| C-Term 26 | NQSKKLLDAADAVKKMLEKEKSS | 20 | 140 |
| C-Term 27 | NQSKKVLETIRWIETVISRQRSS | 20 | 141 |
| C-Term 28 | NQSADLKKVAELVKKLMEEAKKKS | 21 | 142 |
| C-Term 29 | NQSTDTMKAARIMKEELKEKS | 18 | 143 |
| C-Term 30 | NQSRKTEEALRRADTIIKQLASKS | 21 | 144 |
| C-Term 31 | NQSKKLKSAADDVKKAKEKS | 17 | 145 |
| C-Term 32 | NQSKELKSAAEDVKKAKEKS | 17 | 146 |
| C-Term 33 | NQSRETKKATENVKTMLTKSKS | 19 | 147 |
| C-Term 34 | NQSLELKKAAKAANTDLTKKS | 18 | 148 |
| C-Term 35 | NQSLELKEAAKAANTDLTKKS | 18 | 149 |
| C-Term 36 | NQSRKLEEIARIVEQKKRTEEKRS | 21 | 150 |
| C-Term 37 | NQSAETKKAIERAREL | 13 | 151 |
| C-Term 38 | NQSRDLKKAAEIAKKS | 13 | 152 |
| C-Term 39 | NQSRTLLETAEIVTRS | 13 | 153 |
| C-Term 40 | NQSRTLLETAEIVKRS | 13 | 154 |
| C-Term 41 | NQSRKLDKAAEYVEKS | 13 | 155 |
| C-Term 42 | NQSKEAKKAIETAKKLS | 14 | 156 |
| C-Term 43 | NQSRKLETAAEKLKQTE | 14 | 157 |
| C-Term 44 | NQSRLMLEAVKIAQSQS | 14 | 158 |
| C-Term 45 | NQSRETKEAAESVKQMES | 15 | 159 |
| C-Term 46 | NQSRRTLKAIEITLKLLS | 15 | 160 |
| C-Term 47 | NQSRRTLTAITRVERKDS | 15 | 161 |
| C-Term 48 | NQSKKLADAADWVETVKSS | 16 | 162 |
| C-Term 49 | NQSKKTHSAIEWVERLVSS | 16 | 163 |
| C-Term 50 | NQSADTKKAAEIAKKLAKS | 16 | 164 |

The native sequence includes the C-terminal alpha-helical segment ISQVNEKINQSLAFIRRSDE (SEQ ID NO: 713).

In context the C-terminal alpha-helix of the modified construct is ISQVNEKINQSREIIRAINIVRKIASEK (SEQ ID NO: 714) and is only nine residues longer than the portion of the native structure known to be helical, and two residues lower than the predicted helical segment. Contact residues are bold and underlined.

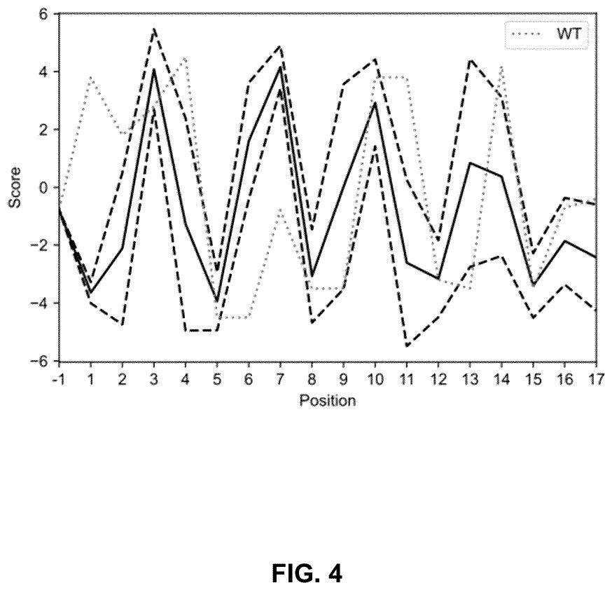

Native
(SEQ ID NO: 715)
ISQVNEKINQSLAFIRRSDELLHNVN
Remodel
(SEQ ID NO: 714)
ISQVNEKINQSREIIRAINIVRKIASEK

Whereas the WT sequence has a three-residue hydrophobic segment leading into the designed helix, and a five-residue polar segment in the middle, which contributes to sub-optimal packing, the remodeled sequences are characterized by a pattern of alternating hydrophobic and polar segments with no hydrophobic segment longer than two consecutive residues and no polar segment longer than three consecutive residues (FIG. 4). The remodeled helix has at minimum two hydrophobic segments at positions 508 and/or 509 and 511 and/or 512 and optimally four hydrophobic segments at positions 505 and/or 506, 508 and/or 509, 511 and/or 512, and 515 and/or 516.

Published structure of the RSV protein generally does not include the residues C-terminal to about residue 500. Either the residues are not included in the recombinant protein studied, or they are not visible in the electronic density observed. Nonetheless, modeling suggests that the following substitutions will stabilize the portion of the F protein in a helical conformation.

TABLE 13
Possible substitutions at Position 505-516

| Position | Preferences suggested by modeling | Illustrative Substitutions |
|---|---|---|
| F505 | Hydrophobic or threonine, not WFY | A, I, L, M, V, G, T; not F, Y, W |
| I506 | Any amino acid except P, preferably | Any amino acids except P; |
| | polar or AILV | preferably D, E K, N, Q, R, S, T, Y |
| | | or A, I, L, V |
| R507 | Any amino acid except P, preferably | Any amino acids except P; |
| | polar or AILV | preferably D, E, K, N, Q, R, S, T, Y |
| | | or A, I, L, V |
| K508 | AVTI preferred, K, Q, R possible | A, V, T, I; possibly K, Q, R |
| S509 | Hydrophobic or Thr. Preferred | A, I, L, M, V, F, W, Y, G, T; |
| | AILVM | preferably A, I, L, M, V |
| D510 | Any amino acid, preferably polar | Any amino acids; preferably D, E, |
| | | K, N, Q, R, S, T, Y |
| E511 | Any amino acid depending on the rest | Any amino acids depending on the |
| | of the design | rest of the design |
| L512 | Preferred hydrophobic, can be T and | Preferably A, I, L, M, V, F, W, Y, G, |
| | in some cases other polar | T; in some cases D, E, K, N, Q, R, S, |
| | | T, Y |
| L513 | Any amino acid, preferred polar but | Any amino acids; preferably D, E, |
| | occasionally hydrophobic | K, N, Q, R, S, T, Y; occasionally A, |
| | | I, L, M, V, F, W, Y, G |
| H514 | Any amino acid except P, preferably | Any amino acids except P; |
| | polar | preferably D, E, K, N, Q, R, S, T, Y |
| N515 | Any amino acid except P, preferably | Any amino acids except P; |
| | hydrophobic | preferably A, I, L, M, V, F, W, Y, G |
| V516 | Hydrophobic or TSK | A, I, L, M, V, F, W, Y, G, or T, S, K |
| N517 | Any amino acid except P, preferably | Any amino acids except P; |
| | polar | preferably D, E, K, N, Q, R, S, T, Y |
| A518 | Any amino acid except P, preferably | Any amino acids except P; |
| | polar | preferably D, E, K, N, Q, R, S, T, Y |
| G519 | Any amino acid except P, preferably | Any amino acids except P; |
| | polar | preferably D, E, K, N, Q, R, S, T, Y |

In some embodiments, polar amino acids refer to D, E, K, N, Q, R, S, T, and Y. In some embodiments, polar amino acids include charged amino acid residues. In some embodiments, charged amino acids refer to E, D, R, K, and H. In some embodiments, hydrophobic amino acids refer to A, I, L, M, V, F, Y, and W.

A small-scale screen showed that three of the four selected designs expressed. Table 14 shows binding of antibodies D25, AM14, and 4D7 to RSV/B F proteins fused to I53-50A to form trimeric protein complexes (but not assembled with I53-50B). Both D25 and AM14 are specific to the prefusion state, however D25 can bind both prefusion monomers and trimers while AM14 can only bind closed trimeric prefusion trimers. 4D7 is specific to the postfusion state. C-Term1 was well expressed and showed the highest binding to AM14.

TABLE 14
Summary of antibody binding screening data for
designed RSV/B F proteins

| Name | Expression | D25 | AM14 | 4D7 |
|---|---|---|---|---|
| C-Term1 | ++ | +++ | +++ | + |
| C-Term 2 | − | NA | NA | NA |
| C-Term 3 | ++ | +++ | ++ | ++ |
| C-Term 4 | +++ | +++ | ++ | + |
| DS-Cav1 | +++ | +++ | ++ | ++ |
| RSV/B.002 | | | | |

### Example 2. Design of Stabilizing Substitutions for RSV F Proteins

This Example describes sets of stabilizing mutations for stabilization of the prefusion state of RSV F protein. Based on a structure of RSV F in the prefusion conformation (FIG. 1) compared to its postfusion conformation (not shown), stabilizing mutations at the interfaces between protomers were designed to either lower the energy of the prefusion state or raise the energy of the postfusion state.

Computational modeling was used to identify amino acid substitutions to stabilize RSV/B F protein in the prefusion conformation. These mutations are listed in Table 15.

TABLE 15
stabilizing substitutions

| Space | Substitutions |
|---|---|
| Space 1 | F140W, K399A, K399V, |
| | T400D, S485I, S485A, S485F, |
| | D486A, D486Q, D486E, D486S, |
| | E487R, E487K, E487A, E487M, |
| | E487Q, 487R, 487M, F488W, |
| | D489A, Q494I, Q494M, Q494L, |
| | Q494A, K498A, K498E, 498A, |
| | 498Y |
| Space 2 | V56L, V56A, T58A, T58S, |
| | T58M, V154I, V187L, V296A, |
| | A298M, A298L, A298I |
| Space 3 | K75Q, N216S, N216D, E218P, |
| | T219S |
| Space 4 | E921, E92A, E232A, E232W, |
| | R235Y, R235W, S238A, S238L, |
| | T249P, Y250F, N254V, N254L |
| Other | T67V, F137D, F137S, R339E |

Based on molecular modeling, combinations of substitutions expected to synergize include:

E487R + K498A
E487R + K498E
E487K + K498E
D486A + E487R + K498A
D486Q + E487R + K498A
D486E + E487A + D489A + T400D
D486A + E487M + K498A
E487Q
D486S
F488W + D489A + T400D + E487R + K498A
F140W + D489A + T400D + E487R + K498A
Q4941 + S4851 + K399A + 487R + 498A
Q494M + S4851 + K399A, D486A + 487M + 498A
Q494L + S485A + K399V + D486A + 487M + 498A
Q494M + S485A + K399V + D486A + 487M + 498A
Q494A + S485F + K399V + D486A + 487M + 498Y
D489A + T400D + E487R + K498A
D489A + T400D

RSV F proteins are cleaved during expression by the protease furin. Constructs that replace the cleavage site for furin (residues 104-140) with a native linker were also tested. Linker sequences are provided in Table 16, which were tested in between residues 103 and 141.

TABLE 16
Furin cleavage linkers

| Sequence | Length | SEQ ID NO: |
|---|---|---|
| NNQARGSGSGRSLGF | 15 | 639 |
| NNQARGGSGGRSLGF | 15 | 640 |
| NNGARGGSGGRSLGF | 15 | 641 |
| NNQARGGSGGDSLGF | 15 | 642 |
| NNQARGGSGSGGDSLGF | 17 | 643 |
| NNQARGGSGGGDLG | 14 | 644 |
| NNQARGGSGSGGDLGF | 16 | 645 |

### Example 3. Experimental Evaluation of RSV F Proteins

This Example shows that the C-terminal helix-forming segments described in Example 1 increase thermal stability of the recombinant polypeptides by as much as about 20-25° C. or more and increase storage stability under accelerated degradation conditions (storage at 40° C.). Further improvement is observed when the C-terminal helix-forming segment is combined with stabilizing mutations described in Example 2. The recombinant polypeptides retain the ability to self-assemble to form a two-component I53-50-type nanostructure.

Recombinant polypeptides that include RSV/B F protein ectodomains (B18537 strain with DS-Cav1 mutations) fused to I53-50AΔcys were tested using small-scale HEK293 expression. Supernatants were screened for relative expression by bio-layer interferometry (BLI) with a monoclonal antibody (16A8) that binds specifically to I53-50A. BLI was used to measure binding to known RSV F protein antibodies D25 (specific to prefusion state), AM14 (specific to closed trimeric prefusion state), and 4D7 (specific to postfusion state). Measurements were normalized to binding by palivizumab (conformation independent). Increased AM14 was observed to several designs featuring mutations in Space 1, C-terminal remodeling, or both.

Scaled-up protein preparation for select designs were incubated for six days at either 4° C. or 40° C. Designs were identified which showed less loss in D25 or AM14 binding at 40° C. compared to DS-Cav1 mutations alone, as well as smaller increases in binding to 4D7 at 40° C. The C-term1 design (Example 1) that includes a remodeled C terminus showed nearly no decrease in AM14 binding and no increase in 4D7 binding.

Sets of mutations were selected for analysis in combination with each other. In these experiments, an ectodomain sequence from a contemporary RSV/B strain was used (hRSV/B/Australia/VIC-RCH056/2019). Antibody binding was normalized to 16A8 mAb, which is specific to the I53-50A fusion partner. Multiple designs were characterized that increased ratios of binding to AM14 (prefusion) or decreased the binding to 4D7 (postfusion) (FIG. 1). Six-day thermal stress tests were performed for select scaled-up proteins.

Fourteen designs were selected for further analysis after scale-up and purification. Antigenic measurements confirmed increases in AM14 binding for all tested designs relative to DS-Cav1 mutations alone. Constructs incorporating C-terminal remodeling generally showed greater thermal stability under storage (i.e., reduced rate of decrease in 4D7 binding).

Constructs selected for thermal denaturation and storage testing are shown in Table 17. All tested RSV/B constructs were based on the sequence of strain hRSV/B/Australia/VIC-RCH056/2019, including the DS-Cav1 mutations, fused to I53-50AΔcys. All proteins were tested as soluble, trimeric fusions (prior to assembly with I53-50B to form a nanostructure). RSV/A.03 (based on the A2 strain) and RSV/B.002 were controls containing the DS-Cav1 substitutions. The data in Table 17 show that the C-terminal alpha-helical segment by itself can increase thermal stability by up to about 25° C. (compare construct RSV/B.002 to RSV/B.195, construct RSV/B.093 to RSV/B.189). Furthermore, all constructs having the C-terminal alpha-helical segment maintain the prefusion conformation when stored at 40° C. for seven days. One construct without the C-terminal alpha-helical segment, RSV/B.093, was also stable prefusion at 40° C., but its melting temperature was lower than constructs containing C-terminal remodeling.

TABLE 17

| | | | Alpha- | NanoDSF | | Storage |
|---|---|---|---|---|---|---|
| | | | helical | Tonset | Tm | Stable at |
| Construct | Serotype | Substitutions⁴ | segment | (° C.) | (° C.) | 40° C. |
| RSV/A.03 | A³ | | | 44.4 | 51.5 | − |
| RSV/B.002 | B¹ | | | 43.4 | 50.1 | − |
| RSV/B.081 | B¹ | D489A | | 51.2 | 56.5 | + |
| | | T400D | | | | |
| | | E487R | | | | |
| | | K498A | | | | |
| | | D486A | | | | |
| RSV/B.093 | B¹ | F488W | | 51.2 | 56.5 | ++ |
| | | D489A | | | | |
| | | T400D | | | | |
| | | E487R | | | | |
| | | K498A | | | | |
| | | D486A | | | | |
| RSV/B.099 | B¹ | E487R | | 43.4 | 50.1 | − |
| | | K498A | | | | |
| | | T67V | | | | |
| RSV/B/100 | B¹ | E487R | | 46.3 | 51.5 | − |
| | | K498A | | | | |
| | | T249P | | | | |
| | | T67V | | | | |
| RSV/B.123 | B¹ | D489A | | 49.9 | 54.9 | + |
| | | T400D | | | | |
| | | E487R | | | | |
| | | K498A | | | | |
| | | T67V | | | | |
| RSV/B.147 | B¹ | E487R | Yes² | 59.0 | 69.7 | ++ |
| | | K498A | | | | |
| RSV/B.148 | B¹ | E487R | Yes² | 64.4 | 77.3 | ++ |
| | | K498A | | | | |
| | | T249P | | | | |
| RSV/B.160 | B¹ | F488W | Yes² | 66.6 | 77.2 | ++ |
| | | D489A | | | | |
| | | T400D | | | | |
| | | E487R | | | | |
| | | K498A | | | | |
| | | T249P | | | | |
| RSV/B.171 | B¹ | D489A | Yes² | 69.0 | 80.9 | ++ |
| | | T400D | | | | |
| | | E487R | | | | |
| | | K498A | | | | |
| RSV/B.172 | B¹ | D489A | Yes² | 65.7 | 77.3 | ++ |
| | | T400D | | | | |
| | | E487R | | | | |
| | | K498A | | | | |
| | | T249P | | | | |
| RSV/B.178 | B¹ | D489A | Yes² | 69.7 | 80.3 | ++ |
| | | T400D | | | | |
| | | E487R | | | | |
| | | K498A | | | | |
| | | D486A | | | | |
| | | T249P | | | | |
| RSV/B.189 | B¹ | F488W | Yes² | 70.8 | 81.1 | ++ |
| | | D489A | | | | |
| | | T400D | | | | |
| | | E487R | | | | |
| | | K498A | | | | |
| | | D486A | | | | |
| RSV/B.195 | B¹ | | Yes² | 56.2 | 68.2 | ++ |
| RSV/A.013 | A³ | | Yes² | 51.6 | 56.0 | ++ |
| RSV/A.023 | A³ | D489A | Yes² | 63.9 | 70.5 | ++ |
| | | T400D | | | | |
| | | E487R | | | | |
| | | K498A | | | | |

¹Based on hRSV/B/Australia/VIC-RCH056/2019 strain
²NOSREIIRAINIVRKIASEK (SEQ ID NO: 10)
³Based on A2 strain
⁴In addition to DS-Cav1 (S155C, S290C, S190F, and V207L)

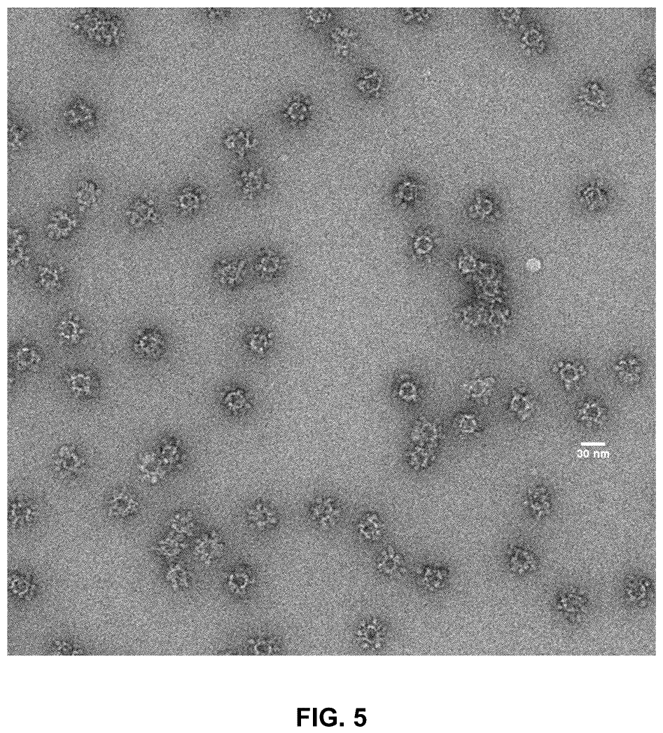

Selected constructs were incubated with a second component, I53-50B, to form nanostructures. Dynamic Light Scattering (DLS) and negative-stain electron microscopy (nsEM) confirm assembly as nanostructure. Results are shown in Table 18. A representative electron micrograph is shown in FIG. 5 (RSV/B.195, having the DS-Cav).

TABLE 18

| | | | | Nanostructure | | |
|---|---|---|---|---|---|---|
| | | | Alpha- | Self- | Compact | |
| | Sero- | Sub- | helical | assembly | trimer | In |
| Construct | type | stitutions² | segment³ | (DLS) | (nsEM) | vivo |
| RSV/A.03 | A | | | Yes | + | Yes |
| RSV/B.002 | B¹ | | | Yes | + | Yes |
| RSV/B.081 | B¹ | D489A | | Yes | Not | No |
| | | T400D | | | tested | |
| | | E487R | | | | |
| | | K498A | | | | |
| | | D486A | | | | |
| RSV/B.093 | B¹ | F488W | | Yes | ++ | Yes |
| | | D489A | | | | |
| | | T400D | | | | |
| | | E487R | | | | |
| | | K498A | | | | |
| | | D486A | | | | |
| RSV/B.099 | B¹ | E487R | | Yes | Not | No |
| | | K498A | | | tested | |
| | | T67V | | | | |
| RSV/B/100 | B¹ | E487R | | Yes | Not | No |
| | | K498A | | | tested | |
| | | T249P | | | | |
| | | T67V | | | | |
| RSV/B.123 | B¹ | D489A | | Yes | Not | No |
| | | T400D | | | tested | |
| | | E487R | | | | |
| | | K498A | | | | |
| | | T67V | | | | |
| RSV/B.147 | B¹ | E487R | Yes | Yes | Not | No |
| | | K498A | | | tested | |
| RSV/B.148 | B¹ | E487R | Yes | Yes | Not | No |
| | | K498A | | | tested | |
| | | T249P | | | | |
| RSV/B.160 | B¹ | F488W | Yes | Yes | ++ | Yes |
| | | D489A | | | | |
| | | T400D | | | | |
| | | E487R | | | | |
| | | K498A | | | | |
| | | T249P | | | | |
| RSV/B.171 | B¹ | D489A | Yes | Yes | ++ | Yes |
| | | T400D | | | | |
| | | E487R | | | | |
| | | K498A | | | | |
| RSV/B.172 | B¹ | D489A | Yes | Yes | Not | No |
| | | T400D | | | tested | |
| | | E487R | | | | |
| | | K498A | | | | |
| | | T249P | | | | |
| RSV/B.178 | B¹ | D489A | Yes | Yes | Not | No |
| | | T400D | | | tested | |
| | | E487R | | | | |
| | | K498A | | | | |
| | | D486A | | | | |
| | | T249P | | | | |
| RSV/B.189 | B¹ | F488W | Yes | Yes | Not | No |
| | | D489A | | | tested | |
| | | T400D | | | | |
| | | E487R | | | | |
| | | K498A | | | | |
| | | D486A | | | | |
| RSV/B.195 | B¹ | | Yes | Yes | ++ | Yes |
| RSV/A.013 | A⁴ | | Yes | Yes | Not | Yes |
| | | | | | tested | |
| RSV/A.023 | A⁴ | D489A | Yes | Yes | Not | Yes |
| | | T400D | | | tested | |
| | | E487R | | | | |
| | | K498A | | | | |

¹Based on hRSV/B/Australia/VIC-RCH056/2019 strain
²In addition to DS-Cav1 (S155C, S290C, S190F, and V207L)
³NOSREIIRAINIVRKIASEK (SEQ ID NO: 10)
⁴Based on A2 strain

Sequences for designed constructs used in Table 18 are shown in Table 19. SEQ ID NO: 1 is used for the reference sequence. In each case, the signal peptide at the N terminus or the tag at the C terminus, shown in underlined may be inserted with known alternatives or deleted. RSV F protein is known to be cleaved at two furin cleavage sites leading to loss of a peptide sequence known as “p27.” (Rezende et al. Front. Microbiol., Vol. 14 (2023).) As used herein, the term “polypeptide” includes polypeptides lacking the p27 peptide due to this cleavage reaction. The approximate region surrounding the p27 peptide is italicized, and may be removed through furin-based cleavage during production of antigens in cell culture.

TABLE 19

| | | SEQ ID |
|---|---|---|
| Construct | Sequence | NO: |
| RSV/A.03 | MELLILKANAITTILTAVTFCFASGQNITEEFYQSTCSAVSKGYLS | 76 |
| | ALRTGWYTSVITIELSNIKENKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSTYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKT | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSARFD | |
| | ASISQVNEAINQSLAFIRKSDELLGSGGSGSGSGGSEKAAKAEEA | |
| | ARKMEELFKKHKIVAVLRANSVEEAIEKAVAVFAGGVHLIEITF | |
| | TVPDADTVIKALSVLKEKGAIIGAGTVTSVEQARKAVESGAEFIV | |
| | SPHLDEEISQFAKEKGVFYMPGVMTPTELVKAMKLGHTILKLFP | |
| | GEVVGPQFVKAMKGPFPNVKFVPTGGVNLDNVAEWFKAGVLA | |
| | VGVGSALVKGTPDEVREKAKAFVEKIRGATELEHHHHHH | |
| RSV/A.013 | MELLILKANAITTILTAVTFCFASGQNITEEFYQSTCSAVSKGYLS | 77 |
| | ALRTGWYTSVITIELSNIKENKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSTYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKT | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSDEFD | |
| | ASISQVNEKINQSREIIRAINIVRKIASEKGSGGSGSGSGGSEKAA | |
| | KAEEAARKMEELFKKHKIVAVLRANSVEEAIEKAVAVFAGGVH | |
| | LIEITFTVPDADTVIKALSVLKEKGAIIGAGTVTSVEQARKAVES | |
| | GAEFIVSPHLDEEISQFAKEKGVFYMPGVMTPTELVKAMKLGHT | |
| | ILKLFPGEVVGPQFVKAMKGPFPNVKFVPTGGVNLDNVAEWFK | |
| | AGVLAVGVGSALVKGTPDEVREKAKAFVEKIRGATELEHHHHH | |
| | H | |
| RSV/A.015 | MELLILKANAITTILTAVTFCFASGQNITEEFYQSTCSAVSKGYLS | 78 |
| | ALRTGWYTSVITIELSNIKENKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSTYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKD | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSARFA | |
| | ASISQVNEAINQSLAFIRKSDELLGSGGSGSGSGGSEKAAKAEEA | |
| | ARKMEELFKKHKIVAVLRANSVEEAIEKAVAVFAGGVHLIEITF | |
| | TVPDADTVIKALSVLKEKGAIIGAGTVTSVEQARKAVESGAEFIV | |
| | SPHLDEEISQFAKEKGVFYMPGVMTPTELVKAMKLGHTILKLFP | |
| | GEVVGPQFVKAMKGPFPNVKFVPTGGVNLDNVAEWFKAGVLA | |
| | VGVGSALVKGTPDEVREKAKAFVEKIRGATELEHHHHHH | |
| RSV/A.016 | MELLILKANAITTILTAVTFCFASGQNITEEFYQSTCSAVSKGYLS | 79 |
| | ALRTGWYTSVITIELSNIKENKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSTYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKD | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSARW | |
| | AASISQVNEAINQSLAFIRKSDELLGSGGSGSGSGGSEKAAKAEE | |
| | AARKMEELFKKHKIVAVLRANSVEEAIEKAVAVFAGGVHLIEIT | |
| | FTVPDADTVIKALSVLKEKGAIIGAGTVTSVEQARKAVESGAEFI | |
| | VSPHLDEEISQFAKEKGVFYMPGVMTPTELVKAMKLGHTILKLF | |
| | PGEVVGPQFVKAMKGPFPNVKFVPTGGVNLDNVAEWFKAGVL | |
| | AVGVGSALVKGTPDEVREKAKAFVEKIRGATELEHHHHHH | |
| RSV/A.017 | MELLILKANAITTILTAVTFCFASGQNITEEFYQSTCSAVSKGYLS | 80 |
| | ALRTGWYTSVITIELSNIKEVKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSTYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKT | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSDRFD | |
| | ASISQVNEAINQSLAFIRKSDELLGSGGSGSGSGGSEKAAKAEEA | |
| | ARKMEELFKKHKIVAVLRANSVEEAIEKAVAVFAGGVHLIEITF | |
| | TVPDADTVIKALSVLKEKGAIIGAGTVTSVEQARKAVESGAEFIV | |
| | SPHLDEEISQFAKEKGVFYMPGVMTPTELVKAMKLGHTILKLFP | |
| | GEVVGPQFVKAMKGPFPNVKFVPTGGVNLDNVAEWFKAGVLA | |
| | VGVGSALVKGTPDEVREKAKAFVEKIRGATELEHHHHHH | |
| RSV/A.018 | MELLILKANAITTILTAVTFCFASGQNITEEFYQSTCSAVSKGYLS | 81 |
| | ALRTGWYTSVITIELSNIKEVKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSPYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKT | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSDRFD | |
| | ASISQVNEAINQSLAFIRKSDELLGSGGSGSGSGGSEKAAKAEEA | |
| | ARKMEELFKKHKIVAVLRANSVEEAIEKAVAVFAGGVHLIEITF | |
| | TVPDADTVIKALSVLKEKGAIIGAGTVTSVEQARKAVESGAEFIV | |
| | SPHLDEEISQFAKEKGVFYMPGVMTPTELVKAMKLGHTILKLFP | |
| | GEVVGPQFVKAMKGPFPNVKFVPTGGVNLDNVAEWFKAGVLA | |
| | VGVGSALVKGTPDEVREKAKAFVEKIRGATELEHHHHHH | |
| RSV/A.019 | MELLILKANAITTILTAVTFCFASGQNITEEFYQSTCSAVSKGYLS | 82 |
| | ALRTGWYTSVITIELSNIKEVKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSTYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKD | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSDRFA | |
| | ASISQVNEAINQSLAFIRKSDELLGSGGSGSGSGGSEKAAKAEEA | |
| | ARKMEELFKKHKIVAVLRANSVEEAIEKAVAVFAGGVHLIEITF | |
| | TVPDADTVIKALSVLKEKGAIIGAGTVTSVEQARKAVESGAEFIV | |
| | SPHLDEEISQFAKEKGVFYMPGVMTPTELVKAMKLGHTILKLFP | |
| | GEVVGPQFVKAMKGPFPNVKFVPTGGVNLDNVAEWFKAGVLA | |
| | VGVGSALVKGTPDEVREKAKAFVEKIRGATELEHHHHHH | |
| RSV/A.020 | MELLILKANAITTILTAVTFCFASGQNITEEFYQSTCSAVSKGYLS | 83 |
| | ALRTGWYTSVITIELSNIKENKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSTYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKT | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSDRFD | |
| | ASISQVNEAINQSREIIRAINIVRKIASEKGSGGSGSGSGGSEKAA | |
| | KAEEAARKMEELFKKHKIVAVLRANSVEEAIEKAVAVFAGGVH | |
| | LIEITFTVPDADTVIKALSVLKEKGAIIGAGTVTSVEQARKAVES | |
| | GAEFIVSPHLDEEISQFAKEKGVFYMPGVMTPTELVKAMKLGHT | |
| | ILKLFPGEVVGPQFVKAMKGPFPNVKFVPTGGVNLDNVAEWFK | |
| | AGVLAVGVGSALVKGTPDEVREKAKAFVEKIRGATELEHHHHH | |
| | H | |
| RSV/A.021 | MELLILKANAITTILTAVTFCFASGQNITEEFYQSTCSAVSKGYLS | 84 |
| | ALRTGWYTSVITIELSNIKENKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSPYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKT | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSDRFD | |
| | ASISQVNEAINQSREIIRAINIVRKIASEKGSGGSGSGSGGSEKAA | |
| | KAEEAARKMEELFKKHKIVAVLRANSVEEAIEKAVAVFAGGVH | |
| | LIEITFTVPDADTVIKALSVLKEKGAIIGAGTVTSVEQARKAVES | |
| | GAEFIVSPHLDEEISQFAKEKGVFYMPGVMTPTELVKAMKLGHT | |
| | ILKLFPGEVVGPQFVKAMKGPFPNVKFVPTGGVNLDNVAEWFK | |
| | AGVLAVGVGSALVKGTPDEVREKAKAFVEKIRGATELEHHHHH | |
| | H | |
| RSV/A.022 | MELLILKANAITTILTAVTFCFASGQNITEEFYQSTCSAVSKGYLS | 85 |
| | ALRTGWYTSVITIELSNIKENKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSPYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKD | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSDRW | |
| | AASISQVNEAINQSREIIRAINIVRKIASEKGSGGSGSGSGGSEKA | |
| | AKAEEAARKMEELFKKHKIVAVLRANSVEEAIEKAVAVFAGGV | |
| | HLIEITFTVPDADTVIKALSVLKEKGAIIGAGTVTSVEQARKAVE | |
| | SGAEFIVSPHLDEEISQFAKEKGVFYMPGVMTPTELVKAMKLGH | |
| | TILKLFPGEVVGPQFVKAMKGPFPNVKFVPTGGVNLDNVAEWF | |
| | KAGVLAVGVGSALVKGTPDEVREKAKAFVEKIRGATELEHHHH | |
| | HH | |
| RSV/A.023 | MELLILKANAITTILTAVTFCFASGQNITEEFYQSTCSAVSKGYLS | 86 |
| | ALRTGWYTSVITIELSNIKENKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSTYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKD | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSDRFA | |
| | ASISQVNEAINQSREIIRAINIVRKIASEKGSGGSGSGSGGSEKAA | |
| | KAEEAARKMEELFKKHKIVAVLRANSVEEAIEKAVAVFAGGVH | |
| | LIEITFTVPDADTVIKALSVLKEKGAIIGAGTVTSVEQARKA VES | |
| | GAEFIVSPHLDEEISQFAKEKGVFYMPGVMTPTELVKAMKLGHT | |
| | ILKLFPGEVVGPQFVKAMKGPFPNVKFVPTGGVNLDNVAEWFK | |
| | AGVLAVGVGSALVKGTPDEVREKAKAFVEKIRGATELEHHHHH | |
| | H | |
| RSV/A.024 | MELLILKANAITTILTAVTFCFASGQNITEEFYQSTCSAVSKGYLS | 87 |
| | ALRTGWYTSVITIELSNIKENKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSPYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKD | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSDRFA | |
| | ASISQVNEAINQSREIIRAINIVRKIASEKGSGGSGSGSGGSEKAA | |
| | KAEEAARKMEELFKKHKIVAVLRANSVEEAIEKAVAVFAGGVH | |
| | LIEITFTVPDADTVIKALSVLKEKGAIIGAGTVTSVEQARKAVES | |
| | GAEFIVSPHLDEEISQFAKEKGVFYMPGVMTPTELVKAMKLGHT | |
| | ILKLFPGEVVGPQFVKAMKGPFPNVKFVPTGGVNLDNVAEWFK | |
| | AGVLAVGVGSALVKGTPDEVREKAKAFVEKIRGATELEHHHHH | |
| | H | |
| RSV/A.025 | MELLILKANAITTILTAVTFCFASGQNITEEFYQSTCSAVSKGYLS | 88 |
| | ALRTGWYTSVITIELSNIKENKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSPYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKD | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSARFA | |
| | ASISQVNEAINQSREIIRAINIVRKIASEKGSGGSGSGSGGSEKAA | |
| | KAEEAARKMEELFKKHKIVAVLRANSVEEAIEKAVAVFAGGVH | |
| | LIEITFTVPDADTVIKALSVLKEKGAIIGAGTVTSVEQARKAVES | |
| | GAEFIVSPHLDEEISQFAKEKGVFYMPGVMTPTELVKAMKLGHT | |
| | ILKLFPGEVVGPQFVKAMKGPFPNVKFVPTGGVNLDNVAEWFK | |
| | AGVLAVGVGSALVKGTPDEVREKAKAFVEKIRGATELEHHHHH | |
| | H | |
| RSV/A.026 | MELLILKANAITTILTAVTFCFASGQNITEEFYQSTCSAVSKGYLS | 89 |
| | ALRTGWYTSVITIELSNIKENKCNGTDAKVKLIKQELDKYKNAV | |
| | TELQLLMQSTPATNNRARRELPRFMNYTLNNAKKTNVTLSKKR | |
| | KRRFLGFLLGVGSAIASGVAVCKVLHLEGEVNKIKSALLSTNKA | |
| | VVSLSNGVSVLTFKVLDLKNYIDKQLLPILNKQSCSISNIETVIEF | |
| | QQKNNRLLEITREFSVNAGVTTPVSTYMLTNSELLSLINDMPITN | |
| | DQKKLMSNNVQIVRQQSYSIMCIIKEEVLAYVVQLPLYGVIDTP | |
| | CWKLHTSPLCTTNTKEGSNICLTRTDRGWYCDNAGSVSFFPQAE | |
| | TCKVQSNRVFCDTMNSLTLPSEVNLCNVDIFNPKYDCKIMTSKD | |
| | DVSSSVITSLGAIVSCYGKTKCTASNKNRGIIKTFSNGCDYVSNK | |
| | GVDTVSVGNTLYYVNKQEGKSLYVKGEPIINFYDPLVFPSARW | |
| | AASISQVNEAINQSREIIRAINIVRKIASEKGSGGSGSGSGGSEKA | |
| | AKAEEAARKMEELFKKHKIVAVLRANSVEEAIEKAVAVFAGGV | |
| | HLIEITFTVPDADTVIKALSVLKEKGAIIGAGTVTSVEQARKAVE | |
| | SGAEFIVSPHLDEEISQFAKEKGVFYMPGVMTPTELVKAMKLGH | |
| | TILKLFPGEVVGPQFVKAMKGPFPNVKFVPTGGVNLDNVAEWF | |
| | KAGVLAVGVGSALVKGTPDEVREKAKAFVEKIRGATELEHHHH | |
| | HH | |
| RSV/B.002 | MELLIHRSSAIFLTLAINALYLTSSQNITEEFYQSTCSAVSRGYLS | 90 |
| | ALRTGWYTSVITIELSNIKETKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSTYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKTDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSDEFDASI | |
| | SQVNEKINQSLAFIRRSDELLGSGGSGSGSGGSEKAAKAEEAAR | |
| | KMEELFKKHKIVAVLRANSVEEAIEKAVAVFAGGVHLIEITFTVP | |
| | DADTVIKALSVLKEKGAIIGAGTVTSVEQARKAVESGAEFIVSPH | |
| | LDEEISQFAKEKGVFYMPGVMTPTELVKAMKLGHTILKLFPGEV | |
| | VGPQFVKAMKGPFPNVKFVPTGGVNLDNVAEWFKAGVLAVGV | |
| | GSALVKGTPDEVREKAKAFVEKIRGATELEHHHHHH | |
| RSV/B.081 | MELLIHRSSAIFLTLAINALYLTSSQNITEEFYQSTCSAVSRGYLS | 91 |
| | ALRTGWYTSVITIELSNIKETKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSTYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKDDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSARFAAS | |
| | ISQVNEAINQSLAFIRRSDELLGSGGSGSGSGGSEKAAKAEEAAR | |
| | KMEELFKKHKIVAVLRANSVEEAIEKAVAVFAGGVHLIEITFTVP | |
| | DADTVIKALSVLKEKGAIIGAGTVTSVEQARKAVESGAEFIVSPH | |
| | LDEEISQFAKEKGVFYMPGVMTPTELVKAMKLGHTILKLFPGEV | |
| | VGPQFVKAMKGPFPNVKFVPTGGVNLDNVAEWFKAGVLAVGV | |
| | GSALVKGTPDEVREKAKAFVEKIRGATELEHHHHHH | |
| RSV/B.093 | MELLIHRSSAIFLTLAINALYLTSSQNITEEFYQSTCSAVSRGYLS | 92 |
| | ALRTGWYTSVITIELSNIKETKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSTYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKDDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSARWAA | |
| | SISQVNEAINQSLAFIRRSDELLGSGGSGSGSGGSEKAAKAEEAA | |
| | RKMEELFKKHKIVAVLRANSVEEAIEKAVAVFAGGVHLIEITFT | |
| | VPDADTVIKALSVLKEKGAIIGAGTVTSVEQARKAVESGAEFIVS | |
| | PHLDEEISQFAKEKGVFYMPGVMTPTELVKAMKLGHTILKLFPG | |
| | EVVGPQFVKAMKGPFPNVKFVPTGGVNLDNVAEWFKAGVLAV | |
| | GVGSALVKGTPDEVREKAKAFVEKIRGATELEHHHHHH | |
| RSV/B.099 | MELLIHRSSAIFLTLAINALYLTSSQNITEEFYQSTCSAVSRGYLS | 93 |
| | ALRTGWYTSVITIELSNIKEVKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSTYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKTDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSDRFDAS | |
| | ISQVNEAINQSLAFIRRSDELLGSGGSGSGSGGSEKAAKAEEAAR | |
| | KMEELFKKHKIVAVLRANSVEEAIEKAVAVFAGGVHLIEITFTVP | |
| | DADTVIKALSVLKEKGAIIGAGTVTSVEQARKAVESGAEFIVSPH | |
| | LDEEISQFAKEKGVFYMPGVMTPTELVKAMKLGHTILKLFPGEV | |
| | VGPQFVKAMKGPFPNVKFVPTGGVNLDNVAEWFKAGVLAVGV | |
| | GSALVKGTPDEVREKAKAFVEKIRGATELEHHHHHH | |
| RSV/B.100 | MELLIHRSSAIFLTLAINALYLTSSQNITEEFYQSTCSAVSRGYLS | 94 |
| | ALRTGWYTSVITIELSNIKEVKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSPYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKTDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSDRFDAS | |
| | ISQVNEAINQSLAFIRRSDELLGSGGSGSGSGGSEKAAKAEEAAR | |
| | KMEELFKKHKIVAVLRANSVEEAIEKAVAVFAGGVHLIEITFTVP | |
| | DADTVIKALSVLKEKGAIIGAGTVTSVEQARKAVESGAEFIVSPH | |
| | LDEEISQFAKEKGVFYMPGVMTPTELVKAMKLGHTILKLFPGEV | |
| | VGPQFVKAMKGPFPNVKFVPTGGVNLDNVAEWFKAGVLAVGV | |
| | GSALVKGTPDEVREKAKAFVEKIRGATELEHHHHHH | |
| RSV/B.123 | MELLIHRSSAIFLTLAINALYLTSSQNITEEFYQSTCSAVSRGYLS | 95 |
| | ALRTGWYTSVITIELSNIKEVKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSTYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKDDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSDRFAAS | |
| | ISQVNEAINQSLAFIRRSDELLGSGGSGSGSGGSEKAAKAEEAAR | |
| | KMEELFKKHKIVAVLRANSVEEAIEKAVAVFAGGVHLIEITFTVP | |
| | DADTVIKALSVLKEKGAIIGAGTVTSVEQARKAVESGAEFIVSPH | |
| | LDEEISQFAKEKGVFYMPGVMTPTELVKAMKLGHTILKLFPGEV | |
| | VGPQFVKAMKGPFPNVKFVPTGGVNLDNVAEWFKAGVLAVGV | |
| | GSALVKGTPDEVREKAKAFVEKIRGATELEHHHHHH | |
| RSV/B.147 | MELLIHRSSAIFLTLAINALYLTSSQNITEEFYQSTCSAVSRGYLS | 96 |
| | ALRTGWYTSVITIELSNIKETKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSTYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKTDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSDRFDAS | |
| | ISQVNEAINQSREIIRAINIVRKIASEKGSGGSGSGSGGSEKAAKA | |
| | EEAARKMEELFKKHKIVAVLRANSVEEAIEKAVAVFAGGVHLIE | |
| | ITFTVPDADTVIKALSVLKEKGAIIGAGTVTSVEQARKAVESGAE | |
| | FIVSPHLDEEISQFAKEKGVFYMPGVMTPTELVKAMKLGHTILK | |
| | LFPGEVVGPQFVKAMKGPFPNVKFVPTGGVNLDNVAEWFKAG | |
| | VLAVGVGSALVKGTPDEVREKAKAFVEKIRGATELEHHHHHH | |
| RSV/B.148 | MELLIHRSSAIFLTLAINALYLTSSQNITEEFYQSTCSAVSRGYLS | 97 |
| | ALRTGWYTSVITIELSNIKETKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSPYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKTDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSDRFDAS | |
| | ISQVNEAINQSREIIRAINIVRKIASEKGSGGSGSGSGGSEKAAKA | |
| | EEAARKMEELFKKHKIVAVLRANSVEEAIEKAVAVFAGGVHLIE | |
| | ITFTVPDADTVIKALSVLKEKGAIIGAGTVTSVEQARKAVESGAE | |
| | FIVSPHLDEEISQFAKEKGVFYMPGVMTPTELVKAMKLGHTILK | |
| | LFPGEVVGPQFVKAMKGPFPNVKFVPTGGVNLDNVAEWFKAG | |
| | VLAVGVGSALVKGTPDEVREKAKAFVEKIRGATELLEHHHHHH | |
| RSV/B.160 | MELLIHRSSAIFLTLAINALYLTSSQNITEEFYQSTCSAVSRGYLS | 98 |
| | ALRTGWYTSVITIELSNIKETKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSPYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKDDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSDRWAA | |
| | SISQVNEAINQSREIIRAINIVRKIASEKGSGGSGSGSGGSEKAAK | |
| | AEEAARKMEELFKKHKIVAVLRANSVEEAIEKAVAVFAGGVHL | |
| | IEITFTVPDADTVIKALSVLKEKGAIIGAGTVTSVEQARKAVESG | |
| | AEFIVSPHLDEEISQFAKEKGVFYMPGVMTPTELVKAMKLGHTI | |
| | LKLFPGEVVGPQFVKAMKGPFPNVKFVPTGGVNLDNVAEWFK | |
| | AGVLAVGVGSALVKGTPDEVREKAKAFVEKIRGATELEHHHHH | |
| | H | |
| RSV/B.171 | MELLIHRSSAIFLTLAINALYLTSSQNITEEFYQSTCSAVSRGYLS | 99 |
| | ALRTGWYTSVITIELSNIKETKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSTYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKDDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSDRFAAS | |
| | ISQVNEAINQSREIIRAINIVRKIASEKGSGGSGSGSGGSEKAAKA | |
| | EEAARKMEELFKKHKIVAVLRANSVEEAIEKAVAVFAGGVHLIE | |
| | ITFTVPDADTVIKALSVLKEKGAIIGAGTVTSVEQARKAVESGAE | |
| | FIVSPHLDEEISQFAKEKGVFYMPGVMTPTELVKAMKLGHTILK | |
| | LFPGEVVGPQFVKAMKGPFPNVKFVPTGGVNLDNVAEWFKAG | |
| | VLAVGVGSALVKGTPDEVREKAKAFVEKIRGATELEHHHHHH | |
| RSV/B.172 | MELLIHRSSAIFLTLAINALYLTSSQNITEEFYQSTCSAVSRGYLS | 100 |
| | ALRTGWYTSVITIELSNIKETKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSPYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKDDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSDRFAAS | |
| | ISQVNEAINQSREIIRAINIVRKIASEKGSGGSGSGSGGSEKAAKA | |
| | EEAARKMEELFKKHKIVAVLRANSVEEAIEKAVAVFAGGVHLIE | |
| | ITFTVPDADTVIKALSVLKEKGAIIGAGTVTSVEQARKAVESGAE | |
| | FIVSPHLDEEISQFAKEKGVFYMPGVMTPTELVKAMKLGHTILK | |
| | LFPGEVVGPQFVKAMKGPFPNVKFVPTGGVNLDNVAEWFKAG | |
| | VLAVGVGSALVKGTPDEVREKAKAFVEKIRGATELEHHHHHH | |
| RSV/B.178 | MELLIHRSSAIFLTLAINALYLTSSQNITEEFYQSTCSAVSRGYLS | 101 |
| | ALRTGWYTSVITIELSNIKETKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSPYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKDDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSARFAAS | |
| | ISQVNEAINQSREIIRAINIVRKIASEKGSGGSGSGSGGSEKAAKA | |
| | EEAARKMEELFKKHKIVAVLRANSVEEAIEKAVAVFAGGVHLIE | |
| | ITFTVPDADTVIKALSVLKEKGAIIGAGTVTSVEQARKAVESGAE | |
| | FIVSPHLDEEISQFAKEKGVFYMPGVMTPTELVKAMKLGHTILK | |
| | LFPGEVVGPQFVKAMKGPFPNVKFVPTGGVNLDNVAEWFKAG | |
| | VLAVGVGSALVKGTPDEVREKAKAFVEKIRGATELEHHHHHH | |
| RSV/B.189 | MELLIHRSSAIFLTLAINALYLTSSQNITEEFYQSTCSAVSRGYLS | 102 |
| | ALRTGWYTSVITIELSNIKETKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSTYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKDDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSARWAA | |
| | SISQVNEAINQSREIIRAINIVRKIASEKGSGGSGSGSGGSEKAAK | |
| | AEEAARKMEELFKKHKIVAVLRANSVEEAIEKAVAVFAGGVHL | |
| | IEITFTVPDADTVIKALSVLKEKGAIIGAGTVTSVEQARKAVESG | |
| | AEFIVSPHLDEEISQFAKEKGVFYMPGVMTPTELVKAMKLGHTI | |
| | LKLFPGEVVGPQFVKAMKGPFPNVKFVPTGGVNLDNVAEWFK | |
| | AGVLAVGVGSALVKGTPDEVREKAKAFVEKIRGATELEHHHHH | |
| | H | |
| RSV/B.195 | MELLIHRSSAIFLTLAINALYLTSSQNITEEFYQSTCSAVSRGYLS | 103 |
| | ALRTGWYTSVITIELSNIKETKCNGTDTKVKLIKQELDKYKNAV | |
| | TELQLLMQNTPAVNNRARREAPQYMNYTINTTKNLNVSISKKRK | |
| | RRFLGFLLGVGSAIASGIAVCKVLHLEGEVNKIKNALQLTNKAV | |
| | VSLSNGVSVLTFRVLDLKNYINNQLLPMLNRQSCRISNIETVIEF | |
| | QQKNSRLLEITREFSVNAGVTTPLSTYMLTNSELLSLINDMPITN | |
| | DQKKLMSSNVQIVRQQSYSIMCIIKEEVLAYVVQLPIYGVIDTPC | |
| | WKLHTSPLCTTNIKEGSNICLTRTDRGWYCDNAGSVSFFPQADT | |
| | CKVQSNRVFCDTMNSLTLPSEVSLCNTDIFNSKYDCKIMTSKTDI | |
| | SSSVITSLGAIVSCYGKTKCTASNKNRGIIKTFSNGCDYVSNKGV | |
| | DTVSVGNTLYYVNKLEGKNLYVKGEPIINYYDPLVFPSDEFDASI | |
| | SQVNEKINQSREIIRAINIVRKIASEKGSGGSGSGSGGSEKAAKAE | |
| | EAARKMEELFKKHKIVAVLRANSVEEAIEKAVAVFAGGVHLIEI | |
| | TFTVPDADTVIKALSVLKEKGAIIGAGTVTSVEQARKAVESGAE | |
| | FIVSPHLDEEISQFAKEKGVFYMPGVMTPTELVKAMKLGHTILK | |
| | LFPGEVVGPQFVKAMKGPFPNVKFVPTGGVNLDNVAEWFKAG | |
| | VLAVGVGSALVKGTPDEVREKAKAFVEKIRGATELEHHHHHH | |

Relative expression and antibody binding of each design are shown in Table 20.

TABLE 20
Relative expression and antibody binding by BLI

| Construct # | Expression | D25 | AM14 | 4D7 | Palivizumab |
|---|---|---|---|---|---|
| RSV/A.03 | +++ | +++ | ++ | ++ | +++ |
| RSV/B.001 | +++ | +++ | ++ | ++ | |
| RSV/B.002 | +++ | +++ | ++ | ++ | +++ |
| RSV/B.008 | + | +++ | ++++ | ++ | |
| RSV/B.030 | ++ | +++ | ++ | ++ | |
| RSV/B.032 | ++ | +++ | ++ | ++ | |
| RSV/B.040 | ++ | +++ | +++ | + | |
| RSV/B.051 | +++ | +++ | +++ | ++ | +++ |
| RSV/B.052 | +++ | +++ | +++ | ++ | +++ |
| RSV/B.053 | ++ | +++ | +++ | ++ | ++ |
| RSV/B.054 | ++ | +++ | ++ | ++ | ++ |
| RSV/B.055 | ++ | +++ | ++ | ++ | +++ |
| RSV/B.056 | + | +++ | ++ | ++ | ++ |
| RSV/B.057 | +++ | +++ | ++++ | ++ | ++ |
| RSV/B.058 | +++ | +++ | ++++ | +++ | ++ |
| RSV/B.059 | + | +++ | +++ | ++ | ++ |
| RSV/B.060 | ++ | +++ | +++ | ++ | ++ |
| RSV/B.061 | ++ | +++ | +++ | + | ++ |
| RSV/B.062 | + | +++ | +++ | +++ | +++ |
| RSV/B.063 | +++ | +++ | +++ | + | +++ |
| RSV/B.064 | +++ | +++ | +++ | ++ | ++++ |
| RSV/B.065 | ++ | +++ | +++ | ++ | ++ |
| RSV/B.066 | +++ | +++ | ++ | ++ | +++ |
| RSV/B.067 | +++ | +++ | ++ | ++ | +++ |
| RSV/B.068 | + | +++ | +++ | ++ | +++ |
| RSV/B.069 | +++ | +++ | +++ | ++ | +++ |
| RSV/B.070 | ++ | +++ | +++ | ++ | ++ |
| RSV/B.071 | + | +++ | +++ | +++ | |
| RSV/B.072 | + | +++ | ++ | +++ | |
| RSV/B.073 | + | +++ | ++ | +++ | |
| RSV/B.074 | + | +++ | +++ | ++++ | |
| RSV/B.075 | +++ | +++ | +++ | + | |
| RSV/B.076 | | +++ | | | |
| RSV/B.077 | ++ | +++ | +++ | + | ++ |
| RSV/B.078 | +++ | +++ | ++ | ++ | |
| RSV/B.079 | +++ | +++ | ++ | ++ | |
| RSV/B.080 | + | +++ | ++ | +++ | |
| RSV/B.081 | ++++ | +++ | ++++ | ++ | |
| RSV/B.082 | +++ | +++ | ++++ | ++ | |
| RSV/B.083 | + | +++ | +++ | ++ | ++ |
| RSV/B.084 | ++ | +++ | +++ | + | |
| RSV/B.085 | ++ | +++ | +++ | + | |
| RSV/B.086 | + | +++ | +++ | +++ | |
| RSV/B.087 | ++++ | +++ | ++++ | ++ | |
| RSV/B.088 | ++++ | +++ | ++++ | ++ | |
| RSV/B.089 | +++ | +++ | +++ | ++ | |
| RSV/B.090 | +++ | +++ | +++ | ++ | |
| RSV/B.091 | +++ | +++ | ++ | + | |
| RSV/B.092 | + | +++ | ++ | ++ | |
| RSV/B.093 | +++ | +++ | ++++ | + | |
| RSV/B.094 | +++ | +++ | ++++ | ++ | |
| RSV/B.095 | ++ | +++ | +++ | ++ | |
| RSV/B.096 | +++ | +++ | ++++ | ++ | |
| RSV/B.097 | +++ | +++ | +++ | ++ | |
| RSV/B.098 | ++ | +++ | +++ | +++ | |
| RSV/B.099 | +++ | +++ | +++ | + | ++ |
| RSV/B.100 | +++ | +++ | +++ | + | ++ |
| RSV/B.101 | ++ | +++ | +++ | + | ++ |
| RSV/B.102 | ++ | +++ | ++ | + | ++ |
| RSV/B.103 | ++ | +++ | ++ | + | ++ |
| RSV/B.104 | + | +++ | +++ | +++ | +++ |
| RSV/B.105 | + | +++ | +++ | +++ | +++ |
| RSV/B.106 | + | +++ | +++ | +++ | +++ |
| RSV/B.107 | + | +++ | +++ | − | + |
| RSV/B.108 | ++ | +++ | ++++ | +++ | ++ |
| RSV/B.109 | ++ | +++ | +++ | + | ++ |
| RSV/B.110 | + | +++ | +++ | +++ | ++ |
| RSV/B.111 | +++ | +++ | +++ | ++ | |
| RSV/B.112 | ++ | +++ | ++ | ++ | +++ |
| RSV/B.113 | + | +++ | ++ | ++++ | +++ |
| RSV/B.114 | + | +++ | ++ | +++ | +++ |
| RSV/B.115 | ++ | +++ | ++ | − | +++ |
| RSV/B.116 | + | +++ | ++ | + | ++ |
| RSV/B.117 | +++ | +++ | +++ | + | ++ |
| RSV/B.118 | ++ | +++ | ++++ | ++ | +++ |
| RSV/B.119 | + | +++ | +++ | ++++ | ++++ |
| RSV/B.120 | + | +++ | ++ | ++++ | +++ |
| RSV/B.121 | + | +++ | ++ | ++++ | +++ |
| RSV/B.122 | + | +++ | ++ | ++++ | +++ |
| RSV/B.123 | ++++ | +++ | +++ | + | +++ |
| RSV/B.124 | ++++ | +++ | +++ | + | +++ |
| RSV/B.125 | ++ | +++ | +++ | ++ | ++ |
| RSV/B.126 | + | +++ | ++ | +++ | +++ |
| RSV/B.127 | + | +++ | ++ | +++ | +++ |
| RSV/B.128 | + | +++ | +++ | ++++ | +++ |
| RSV/B.129 | + | +++ | +++ | +++ | +++ |
| RSV/B.130 | + | +++ | +++ | +++ | +++ |
| RSV/B.131 | + | +++ | +++ | ++ | +++ |
| RSV/B.132 | + | +++ | +++ | +++ | +++ |
| RSV/B.133 | + | +++ | +++ | +++ | +++ |
| RSV/B.134 | + | +++ | ++ | ++++ | +++ |
| RSV/B.135 | + | +++ | +++ | +++ | +++ |
| RSV/B.136 | + | +++ | ++ | ++ | +++ |
| RSV/B.137 | + | +++ | ++ | ++++ | +++ |
| RSV/B.138 | + | +++ | ++ | ++++ | +++ |
| RSV/B.139 | ++ | +++ | ++ | ++ | +++ |
| RSV/B.140 | + | +++ | ++ | +++ | +++ |
| RSV/B.141 | ++ | +++ | +++ | ++ | +++ |
| RSV/B.142 | ++ | +++ | ++ | ++ | +++ |
| RSV/B.143 | + | +++ | ++ | +++ | +++ |
| RSV/B.144 | + | +++ | ++ | +++ | +++ |
| RSV/B.145 | + | +++ | ++ | +++ | +++ |
| RSV/B.146 | + | +++ | ++ | ++++ | ++++ |
| RSV/B.147 | ++++ | +++ | +++ | + | N/A |
| RSV/B.148 | ++++ | +++ | +++ | + | N/A |
| RSV/B.149 | + | +++ | ++ | ++ | N/A |
| RSV/B.150 | ++ | +++ | +++ | − | N/A |
| RSV/B.151 | ++ | +++ | ++++ | − | N/A |
| RSV/B.152 | + | ++++ | +++ | − | N/A |
| RSV/B.153 | +++ | +++ | +++ | + | N/A |
| RSV/B.154 | +++ | +++ | +++ | + | N/A |
| RSV/B.155 | + | +++ | ++ | + | N/A |
| RSV/B.156 | ++ | +++ | +++ | + | N/A |
| RSV/B.157 | + | +++ | +++ | + | N/A |
| RSV/B.158 | + | +++ | ++ | +++ | N/A |
| RSV/B.159 | +++ | +++ | +++ | ++ | N/A |
| RSV/B.160 | ++++ | +++ | +++ | + | N/A |
| RSV/B.161 | ++ | +++ | ++ | − | N/A |
| RSV/B.162 | ++ | ++++ | ++++ | − | N/A |
| RSV/B.163 | +++ | +++ | ++ | + | N/A |
| RSV/B.164 | ++ | +++ | ++ | + | N/A |
| RSV/B.165 | +++ | +++ | +++ | + | N/A |
| RSV/B.166 | ++ | +++ | ++ | +++ | N/A |
| RSV/B.167 | + | +++ | ++ | − | N/A |
| RSV/B.168 | + | +++ | ++ | − | N/A |
| RSV/B.169 | + | + | + | − | N/A |
| RSV/B.170 | + | +++ | + | − | N/A |
| RSV/B.171 | +++ | +++ | +++ | + | N/A |
| RSV/B.172 | ++++ | +++ | +++ | + | N/A |
| RSV/B.173 | ++ | +++ | +++ | +++ | N/A |
| RSV/B.174 | +++ | +++ | +++ | ++ | N/A |
| RSV/B.175 | ++ | +++ | ++ | +++ | N/A |
| RSV/B.176 | + | +++ | ++ | +++ | N/A |
| RSV/B.177 | ++ | +++ | +++ | +++ | N/A |
| RSV/B.178 | +++ | +++ | +++ | + | N/A |
| RSV/B.179 | + | +++ | ++ | ++ | N/A |
| RSV/B.180 | +++ | +++ | +++ | + | N/A |
| RSV/B.181 | ++ | +++ | ++ | + | N/A |
| RSV/B.182 | ++ | ++ | ++ | + | N/A |
| RSV/B.183 | +++ | +++ | ++ | ++ | N/A |
| RSV/B.184 | ++++ | +++ | +++ | + | N/A |
| RSV/B.185 | ++ | +++ | ++ | ++ | N/A |
| RSV/B.186 | ++ | +++ | ++ | + | N/A |
| RSV/B.187 | ++ | +++ | ++ | + | N/A |
| RSV/B.188 | ++ | +++ | ++ | +++ | N/A |
| RSV/B.189 | ++++ | +++ | +++ | − | N/A |
| RSV/B.190 | ++++ | +++ | +++ | + | N/A |
| RSV/B.191 | ++ | +++ | ++ | ++ | N/A |
| RSV/B.192 | ++ | +++ | +++ | + | N/A |
| RSV/B.193 | + | + | + | − | N/A |
| RSV/B.194 | + | ++ | + | + | N/A |

Mutations of designed constructs used in the experiments are shown in Table 21. All sequences featured the ectodomain of RSV F (with DS-Cav1 mutations) genetically fused to I53-50AΔcys (SEQ ID NO: 64) with a flexible glycine- and serine-based linker. Designs that contain a C-terminal alpha-helical segment place this segment at the C-terminus of the ectodomain as described earlier, and prior to the flexible linker. SEQ ID NO: 1 is used for the reference sequence. In each case, the signal peptide at the N terminus or the tag at the C terminus may be replaced with known alternatives or deleted. “o” indicated that an amino acid substitution was used.

TABLE 21
Mutations of constructs used in the experiments

| | | | | | | Alpha- |
|---|---|---|---|---|---|---|
| Construct | | T58M V154I | | R235Y | | helical |
| # | Space 1 | V296A A298L | T249P | E232A | T67V | segment¹ |
| RSV/A.03 | | | | | | |
| RSV/B.001 | | | | | | |
| RSV/B.002 | | | | | | |
| RSV/B.008 | D486A + E487R + K498A | | | | | |
| RSV/B.030 | | | ○ | | | |
| RSV/B.032 | | | ○ | ○ | | |
| RSV/B.040 | | | | | | ◯ |
| RSV/B.051 | E487R + K498A | | | | | |
| RSV/B.052 | E487R + K498A | | ○ | | | |
| RSV/B.053 | E487R + K498A | | ○ | ○ | | |
| RSV/B.054 | E487R + K498A | ○ | | | | |
| RSV/B.055 | E487R + K498A | ○ | ○ | | | |
| RSV/B.056 | E487R + K498A | ○ | ○ | ○ | | |
| RSV/B.057 | D486A + E487R + K498A | | | | | |
| RSV/B.058 | D486A + E487R + K498A | | ○ | | | |
| RSV/B.059 | D486A + E487R + K498A | | ○ | ○ | | |
| RSV/B.060 | D486A + E487R + K498A | ○ | | | | |
| RSV/B.061 | D486A + E487R + K498A | ○ | ○ | | | |
| RSV/B.062 | D486A + E487R + K498A | ○ | ○ | ○ | | |
| RSV/B.063 | F488W + D489A + T400D + | | | | | |
| | E487R + K498A | | | | | |
| RSV/B.064 | F488W + D489A + T400D + | | ○ | | | |
| | E487R + K498A | | | | | |
| RSV/B.065 | F488W + D489A + T400D + | | ○ | ○ | | |
| | E487R + K498A | | | | | |
| RSV/B.066 | F488W + D489A + T400D + | ○ | | | | |
| | E487R + K498A | | | | | |
| RSV/B.067 | F488W + D489A + T400D + | ○ | ○ | | | |
| | E487R + K498A | | | | | |
| RSV/B.068 | F488W + D489A + T400D + | ○ | ○ | ○ | | |
| | E487R + K498A | | | | | |
| RSV/B.069 | Q494M, S485I, K399A, | | | | | |
| | D486A + 487M + 498A | | | | | |
| RSV/B.070 | Q494M, S485I, K399A, | | ○ | | | |
| | D486A + 487M + 498A | | | | | |
| RSV/B.071 | Q494M, S485I, K399A, | | ○ | ○ | | |
| | D486A + 487M + 498A | | | | | |
| RSV/B.072 | Q494M, S485I, K399A, | ○ | | | | |
| | D486A + 487M + 498A | | | | | |
| RSV/B.073 | Q494M, S485I, K399A, | ○ | ○ | | | |
| | D486A + 487M + 498A | | | | | |
| RSV/B.074 | Q494M, S485I, K399A, | ○ | ○ | ○ | | |
| | D486A + 487M + 498A | | | | | |
| RSV/B.075 | D489A + T400D + E487R + | | | | | |
| | K498A | | | | | |
| RSV/B.076 | D489A + T400D + E487R + | | ○ | | | |
| | K498A | | | | | |
| RSV/B.077 | D489A + T400D + E487R + | | ○ | ○ | | |
| | K498A | | | | | |
| RSV/B.078 | D489A + T400D + E487R + | ○ | | | | |
| | K498A | | | | | |
| RSV/B.079 | D489A + T400D + E487R + | ○ | ○ | | | |
| | K498A | | | | | |
| RSV/B.080 | D489A + T400D + E487R + | ○ | ○ | ○ | | |
| | K498A | | | | | |
| RSV/B.081 | D489A + T400D + E487R + | | | | | |
| | K498A + D486A | | | | | |
| RSV/B.082 | D489A + T400D + E487R + | | ○ | | | |
| | K498A + D486A | | | | | |
| RSV/B.083 | D489A + T400D + E487R + | | ○ | ○ | | |
| | K498A + D486A | | | | | |
| RSV/B.084 | D489A + T400D + E487R + | ○ | | | | |
| | K498A + D486A | | | | | |
| RSV/B.085 | D489A + T400D + E487R + | ○ | ○ | | | |
| | K498A + D486A | | | | | |
| RSV/B.086 | D489A + T400D + E487R + | ○ | ○ | ○ | | |
| | K498A + D486A | | | | | |
| RSV/B.087 | F140W + D489A + T400D + | | | | | |
| | E487R + K498A + D486A | | | | | |
| RSV/B.088 | F140W + D489A + T400D + | | ○ | | | |
| | E487R + K498A + D486A | | | | | |
| RSV/B.089 | F140W + D489A + T400D + | | ○ | ○ | | |
| | E487R + K498A + D486A | | | | | |
| RSV/B.090 | F140W + D489A + T400D + | ○ | | | | |
| | E487R + K498A + D486A | | | | | |
| RSV/B.091 | F140W + D489A + T400D + | ○ | ○ | | | |
| | E487R + K498A + D486A | | | | | |
| RSV/B.092 | F140W + D489A + T400D + | ○ | ○ | ○ | | |
| | E487R + K498A + D486A | | | | | |
| RSV/B.093 | F488W + D489A + T400D + | | | | | |
| | E487R + K498A + D486A | | | | | |
| RSV/B.094 | F488W + D489A + T400D + | | ○ | | | |
| | E487R + K498A + D486A | | | | | |
| RSV/B.095 | F488W + D489A + T400D + | | ○ | ○ | | |
| | E487R + K498A + D486A | | | | | |
| RSV/B.096 | F488W + D489A + T400D + | ○ | | | | |
| | E487R + K498A + D486A | | | | | |
| RSV/B.097 | F488W + D489A + T400D + | ○ | ○ | | | |
| | E487R + K498A + D486A | | | | | |
| RSV/B.098 | F488W + D489A + T400D + | ○ | ○ | ○ | | |
| | E487R + K498A + D486A | | | | | |
| RSV/B.099 | E487R + K498A | | | | ○ | |
| RSV/B.100 | E487R + K498A | | ○ | | ○ | |
| RSV/B.101 | E487R + K498A | | ○ | ○ | ○ | |
| RSV/B.102 | E487R + K498A | ○ | | | ○ | |
| RSV/B.103 | E487R + K498A | ○ | ○ | | ○ | |
| RSV/B.104 | E487R + K498A | ○ | ○ | ○ | ○ | |
| RSV/B.105 | D486A + E487R + K498A | | | | ○ | |
| RSV/B.106 | D486A + E487R + K498A | | ○ | | ○ | |
| RSV/B.107 | D486A + E487R + K498A | | ○ | ○ | ○ | |
| RSV/B.108 | D486A + E487R + K498A | ○ | | | ○ | |
| RSV/B.109 | D486A + E487R + K498A | ○ | ○ | | ○ | |
| RSV/B.110 | D486A + E487R + K498A | ○ | ○ | ○ | ○ | |
| RSV/B.111 | F488W + D489A + T400D + | | | | ○ | |
| | E487R + K498A | | | | | |
| RSV/B.112 | F488W + D489A + T400D + | | ○ | | ○ | |
| | E487R + K498A | | | | | |
| RSV/B.113 | F488W + D489A + T400D + | | ○ | ○ | ○ | |
| | E487R + K498A | | | | | |
| RSV/B.114 | F488W + D489A + T400D + | ○ | | | ○ | |
| | E487R + K498A | | | | | |
| RSV/B.115 | F488W + D489A + T400D + | ○ | ○ | | ○ | |
| | E487R + K498A | | | | | |
| RSV/B.116 | F488W + D489A + T400D + | ○ | ○ | ○ | ○ | |
| | E487R + K498A | | | | | |
| RSV/B.117 | Q494M, S485I, K399A, | | | | ○ | |
| | D486A + 487M + 498A | | | | | |
| RSV/B.118 | Q494M, S485I, K399A, | | ○ | | ○ | |
| | D486A + 487M + 498A | | | | | |
| RSV/B.119 | Q494M, S485I, K399A, | | ○ | ○ | ○ | |
| | D486A + 487M + 498A | | | | | |
| RSV/B.120 | Q494M, S485I, K399A, | ○ | | | ○ | |
| | D486A + 487M + 498A | | | | | |
| RSV/B.121 | Q494M, S485I, K399A, | ○ | ○ | | ○ | |
| | D486A + 487M + 498A | | | | | |
| RSV/B.122 | Q494M, S485I, K399A, | ○ | ○ | ○ | ○ | |
| | D486A + 487M + 498A | | | | | |
| RSV/B.123 | D489A + T400D + E487R + | | | | ○ | |
| | K498A | | | | | |
| RSV/B.124 | D489A + T400D + E487R + | | ○ | | ○ | |
| | K498A | | | | | |
| RSV/B.125 | D489A + T400D + E487R + | | ○ | ○ | ○ | |
| | K498A | | | | | |
| RSV/B.126 | D489A + T400D + E487R + | ○ | | | ○ | |
| | K498A | | | | | |
| RSV/B.127 | D489A + T400D + E487R + | ○ | ○ | | ○ | |
| | K498A | | | | | |
| RSV/B.128 | D489A + T400D + E487R + | ○ | ○ | ○ | ○ | |
| | K498A | | | | | |
| RSV/B.129 | D489A + T400D + E487R + | | | | ○ | |
| | K498A + D486A | | | | | |
| RSV/B.130 | D489A + T400D + E487R + | | ○ | | ○ | |
| | K498A + D486A | | | | | |
| RSV/B.131 | D489A + T400D + E487R + | | ○ | ○ | ○ | |
| | K498A + D486A | | | | | |
| RSV/B.132 | D489A + T400D + E487R + | ○ | | | ○ | |
| | K498A + D486A | | | | | |
| RSV/B.133 | D489A + T400D + E487R + | ○ | ○ | | ○ | |
| | K498A + D486A | | | | | |
| RSV/B.134 | D489A + T400D + E487R + | ○ | ○ | ○ | ○ | |
| | K498A + D486A | | | | | |
| RSV/B.135 | F140W + D489A + T400D + | | | | ○ | |
| | E487R + K498A + D486A | | | | | |
| RSV/B.136 | F140W + D489A + T400D + | | ○ | | ○ | |
| | E487R + K498A + D486A | | | | | |
| RSV/B.137 | F140W + D489A + T400D + | | ○ | ○ | ○ | |
| | E487R + K498A + D486A | | | | | |
| RSV/B.138 | F140W + D489A + T400D + | ○ | | | ○ | |
| | E487R + K498A + D486A | | | | | |
| RSV/B.139 | F140W + D489A + T400D + | ○ | ○ | | ○ | |
| | E487R + K498A + D486A | | | | | |
| RSV/B.140 | F140W + D489A + T400D + | ○ | ○ | ○ | ○ | |
| | E487R + K498A + D486A | | | | | |
| RSV/B.141 | F488W + D489A + T400D + | | | | ○ | |
| | E487R + K498A + D486A | | | | | |
| RSV/B.142 | F488W + D489A + T400D + | | ○ | | ○ | |
| | E487R + K498A + D486A | | | | | |
| RSV/B.143 | F488W + D489A + T400D + | | ○ | ○ | ○ | |
| | E487R + K498A + D486A | | | | | |
| RSV/B.144 | F488W + D489A + T400D + | ○ | | | ○ | |
| | E487R + K498A + D486A | | | | | |
| RSV/B.145 | F488W + D489A + T400D + | ○ | ○ | | ○ | |
| | E487R + K498A + D486A | | | | | |
| RSV/B.146 | F488W + D489A + T400D + | ○ | ○ | ○ | ○ | |
| | E487R + K498A + D486A | | | | | |
| RSV/B.147 | E487R + K498A | | | | | ◯ |
| RSV/B.148 | E487R + K498A | | ○ | | | ◯ |
| RSV/B.149 | E487R + K498A | | ○ | ○ | | ◯ |
| RSV/B.150 | E487R + K498A | ○ | | | | ◯ |
| RSV/B.151 | E487R + K498A | ○ | ○ | | | ◯ |
| RSV/B.152 | E487R + K498A | ○ | ○ | ○ | | ◯ |
| RSV/B.153 | D486A + E487R + K498A | | | | | ◯ |
| RSV/B.154 | D486A + E487R + K498A | | ○ | | | ◯ |
| RSV/B.155 | D486A + E487R + K498A | | ○ | ○ | | ◯ |
| RSV/B.156 | D486A + E487R + K498A | ○ | | | | ◯ |
| RSV/B.157 | D486A + E487R + K498A | ○ | ○ | | | ◯ |
| RSV/B.158 | D486A + E487R + K498A | ○ | ○ | ○ | | ◯ |
| RSV/B.159 | F488W + D489A + T400D + | | | | | ◯ |
| | E487R + K498A | | | | | |
| RSV/B.160 | F488W + D489A + T400D + | | ○ | | | ◯ |
| | E487R + K498A | | | | | |
| RSV/B.161 | F488W + D489A + T400D + | | ○ | ○ | | ◯ |
| | E487R + K498A | | | | | |
| RSV/B.162 | F488W + D489A + T400D + | ○ | | | | ◯ |
| | E487R + K498A | | | | | |
| RSV/B.163 | F488W + D489A + T400D + | ○ | ○ | | | ◯ |
| | E487R + K498A | | | | | |
| RSV/B.164 | F488W + D489A + T400D + | ○ | ○ | ○ | | ◯ |
| | E487R + K498A | | | | | |
| RSV/B.165 | Q494M, S485I, K399A, | | | | | ◯ |
| | D486A + 487M + 498A | | | | | |
| RSV/B.166 | Q494M, S485I, K399A, | | ○ | | | ◯ |
| | D486A + 487M + 498A | | | | | |
| RSV/B.167 | Q494M, S485I, K399A, | | ○ | ○ | | ◯ |
| | D486A + 487M + 498A | | | | | |
| RSV/B.168 | Q494M, S485I, K399A, | ○ | | | | ◯ |
| | D486A + 487M + 498A | | | | | |
| RSV/B.169 | Q494M, S485I, K399A, | ○ | ○ | | | ◯ |
| | D486A + 487M + 498A | | | | | |
| RSV/B.170 | Q494M, S485I, K399A, | ○ | ○ | ○ | | ◯ |
| | D486A + 487M + 498A | | | | | |
| RSV/B.171 | D489A + T400D + E487R + | | | | | ◯ |
| | K498A | | | | | |
| RSV/B.172 | D489A + T400D + E487R + | | ○ | | | ◯ |
| | K498A | | | | | |
| RSV/B.173 | D489A + T400D + E487R + | | ○ | ○ | | ◯ |
| | K498A | | | | | |
| RSV/B.174 | D489A + T400D + E487R + | ○ | | | | ◯ |
| | K498A | | | | | |
| RSV/B.175 | D489A + T400D + E487R + | ○ | ○ | | | ◯ |
| | K498A | | | | | |
| RSV/B.176 | D489A + T400D + E487R + | ○ | ○ | ○ | | ◯ |
| | K498A | | | | | |
| RSV/B.177 | D489A + T400D + E487R + | | | | | ◯ |
| | K498A + D486A | | | | | |
| RSV/B.178 | D489A + T400D + E487R + | | ○ | | | ◯ |
| | K498A + D486A | | | | | |
| RSV/B.179 | D489A + T400D + E487R + | | ○ | ○ | | ◯ |
| | K498A + D486A | | | | | |
| RSV/B.180 | D489A + T400D + E487R + | ○ | | | | ◯ |
| | K498A + D486A | | | | | |
| RSV/B.181 | D489A + T400D + E487R + | ○ | ○ | | | ◯ |
| | K498A + D486A | | | | | |
| RSV/B.182 | D489A + T400D + E487R + | ○ | ○ | ○ | | ◯ |
| | K498A + D486A | | | | | |
| RSV/B.183 | F140W + D489A + T400D + | | | | | ◯ |
| | E487R + K498A + D486A | | | | | |
| RSV/B.184 | F140W + D489A + T400D + | | ○ | | | ◯ |
| | E487R + K498A + D486A | | | | | |
| RSV/B.185 | F140W + D489A + T400D + | | ○ | ○ | | ◯ |
| | E487R + K498A + D486A | | | | | |
| RSV/B.186 | F140W + D489A + T400D + | ○ | | | | ◯ |
| | E487R + K498A + D486A | | | | | |
| RSV/B.187 | F140W + D489A + T400D + | ○ | ○ | | | ◯ |
| | E487R + K498A + D486A | | | | | |
| RSV/B.188 | F140W + D489A + T400D + | ○ | ○ | ○ | | ◯ |
| | E487R + K498A + D486A | | | | | |
| RSV/B.189 | F488W + D489A + T400D + | | | | | ◯ |
| | E487R + K498A + D486A | | | | | |
| RSV/B.190 | F488W + D489A + T400D + | | ○ | | | ◯ |
| | E487R + K498A + D486A | | | | | |
| RSV/B.191 | F488W + D489A + T400D + | | ○ | ○ | | ◯ |
| | E487R + K498A + D486A | | | | | |
| RSV/B.192 | F488W + D489A + T400D + | ○ | | | | ◯ |
| | E487R + K498A + D486A | | | | | |
| RSV/B.193 | F488W + D489A + T400D + | ○ | ○ | | | ◯ |
| | E487R + K498A + D486A | | | | | |
| RSV/B.194 | F488W + D489A + T400D + | ○ | ○ | ○ | | ◯ |
| | E487R + K498A + D486A | | | | | |
| RSV/B.195 | | | | | | ◯ |
| RSV/A.013 | | | | | | ◯ |
| RSV/A.023 | D489A + T400D + E487R + | | | | | ◯ |
| | K498A | | | | | |

¹500-NQSREIIRAINIVRKIASEK-519

To test whether these stabilizing modifications are generalizable outside of RSV/B-based antigens, two novel designs were also evaluated in the context of an RSV/A antigen sequence (RSV/A.013 and RSV/A.023). Both designs contained DS-Cav1 mutations and were genetically fused to I53-50AΔcys, with RSV/A.013 adding a C-terminal alpha-helical segment (equivalent to the RSV/B.195 design) and RSV/A.023 adding both a C-terminal alpha-helical segment and D489A, T400D, E487R and K498A mutations (equivalent to the RSV/B.171 design). Sequences and mutations for these designs are further detailed in Table 19 and Table 21 respectively. Both thermal stability and storage stability at 40° C. were strongly increased relative to the RSV/A.03 design, which did not include a C-terminal alpha-helical segment or D489A, T400D, E487R and K498A mutations (Table 17). RSV/A.013 and RSV/A.023 showed increases in melting temperature of 4.5° C. and 19.0° C. relative to RSV/A.03, which demonstrates that the C-terminal alpha-helical segment can be alone used to improve the thermal stability of both RSV/A and RSV/B antigens, and that the combination of the C-terminal alpha-helical segment with further stabilizing mutations can more rigorously improve the thermal stability of both RSV/A and RSV/B antigens. Further, both RSV/A.013 and RSV/A.023 were capable of in vitro assembly into nanostructures with addition of I53-50B as evaluated by DLS (Table 18).

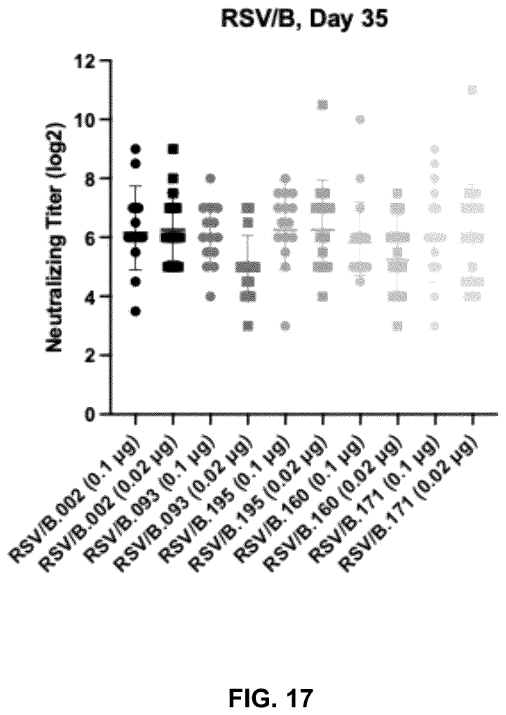

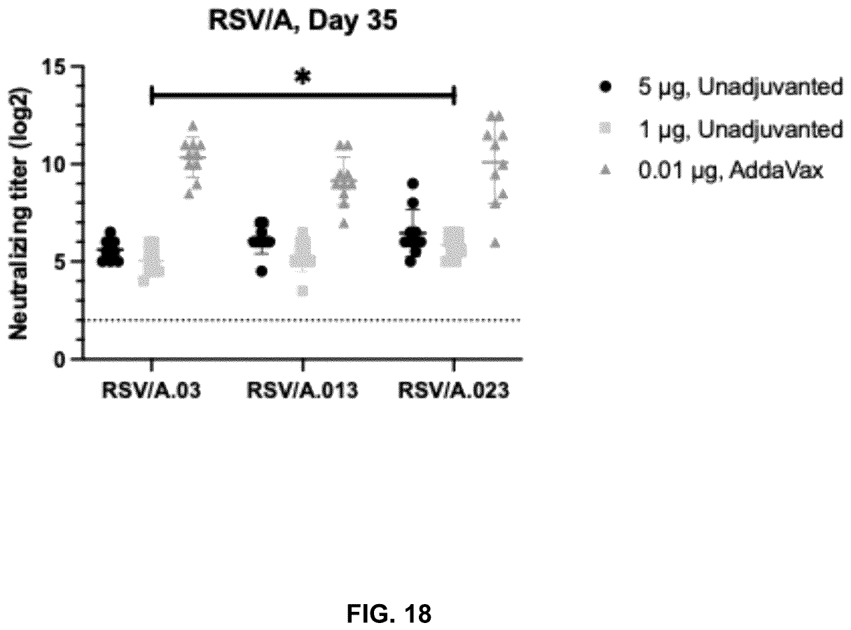

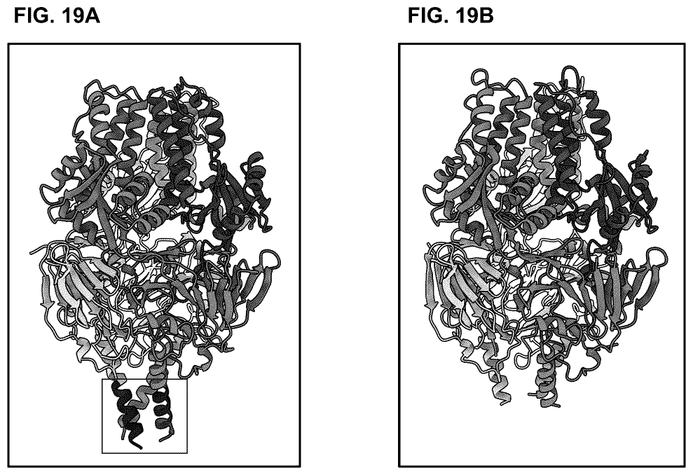

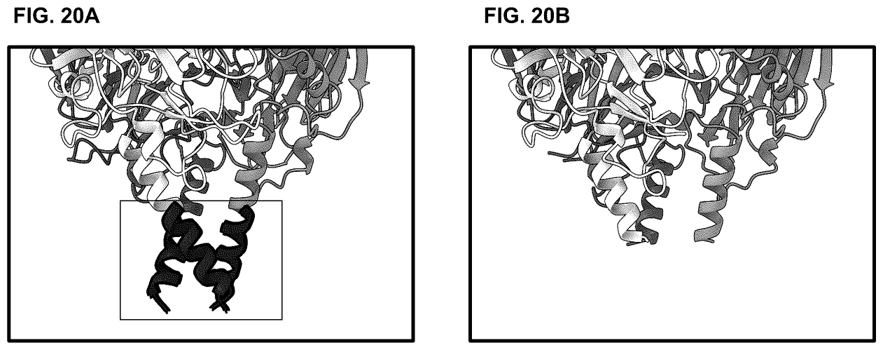

In order to evaluate the immunogenicity of different designs based on either RSV/B or RSV/A, two in vivo studies were performed in BALB/c mice. In one study, RSV/B neutralizing titers elicited by immunization with either a 0.02 mg or 0.1 mg dose of assembled nanostructures based on RSV/B.002, RSV/B.093, RSV/B.195, RSV/B.160, or RSV/B.171 were evaluated, all of which were adjuvanted with Adda Vax (FIG. 17). No statistically significant differences between any of the designs were observed at either dose. Similarly, no statistically significant differences were observed between mice immunized with either a 5 mg unadjuvanted or 0.01 mg Adda Vax-adjuvanted dose of assembled nanostructures based on RSV/A.03, RSV/A.013, or RSV/A.023 (FIG. 18). However, mice immunized with 1 mg of unadjuvanted RSV/A.023 nanostructure did have significantly higher RSV/A neutralizing titers than mice immunized with the same dose of unadjuvanted RSV/A.03.

### Example 4. Diffusion Methods to Generate a C Terminus

Relaxed structures used as input for Rosetta Remodel were also used as input for RFdiffusion, except that only the C-terminal helices and neighboring residues were used as input for diffusion. This significantly reduces computational time. Positions with WT sequence identities from Rosetta Remodel were likewise preserved for diffusion. Unlike with Rosetta Remodel all structural data of subsequent residues was ignored. This is a limitation of RFdiffusion, not a scientific constraint placed on the design problem. Intra-protomer and inter-protomer weights were both set to 1, with a quadratic guide decay and a guide scale of 2. The non-standard weights Base_epoch8_ckpt.pt were applied and C3 symmetry was enforced. C-terminal lengths varied between 12 and 31 residues depending on the source virus, and 15 remodeled helices were generated for each length. Sequences were generated using Protein MPNN, with a sampling temperature of 0.4 and a negative bias against C (−10), and F, G, P, W, and Y (−1). 100 sequences were generated for each remodeled domain. The top 2% based on MPNN sample score were selected for computational characterization.

A set of unique all alpha-helical bundles were generated for each input structure. For most inputs, Rosetta Remodel (Remodel) and RFDiffusion (Diffusion) were both used, except for PIV5 where Remodel generated ample unique results. The number and quality of the output structures was highly variable, depending on the input structure. For example, the C-terminal residues in most structures suffer from low data quality, likely due to local flexibility. This, combined with consistent evidence for a lack of effort in refining this region, may have resulted in sub-optimal bond angles and lengths. Furthermore, many fusion proteins are slightly asymmetric. Symmetrization could have introduced strain. Collectively these effects can influence the quality and number of outputs passing the ddG filter, and also the results generated by diffusion. For that reason, both remodel and diffusion were used where remodel alone was not sufficient to generate enough quality outputs.

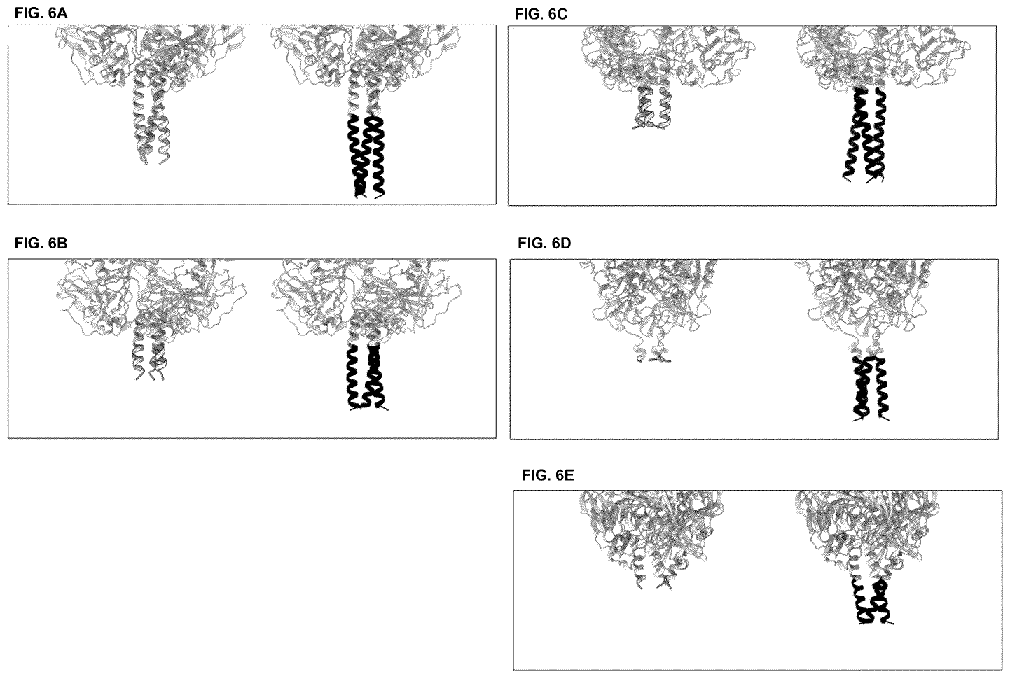

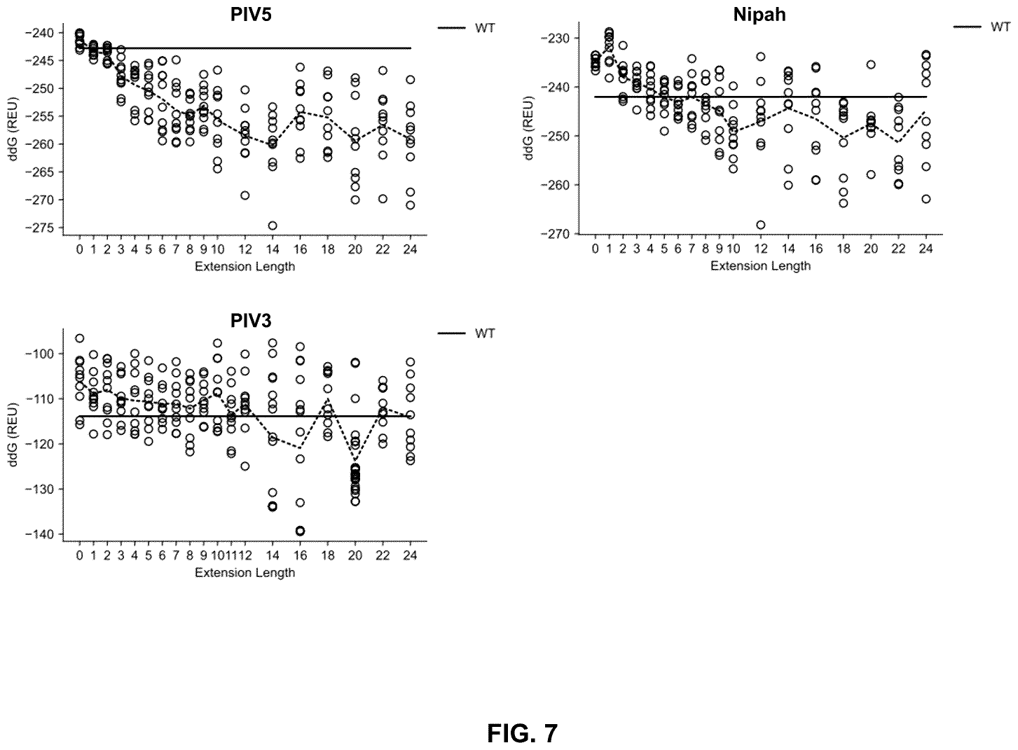

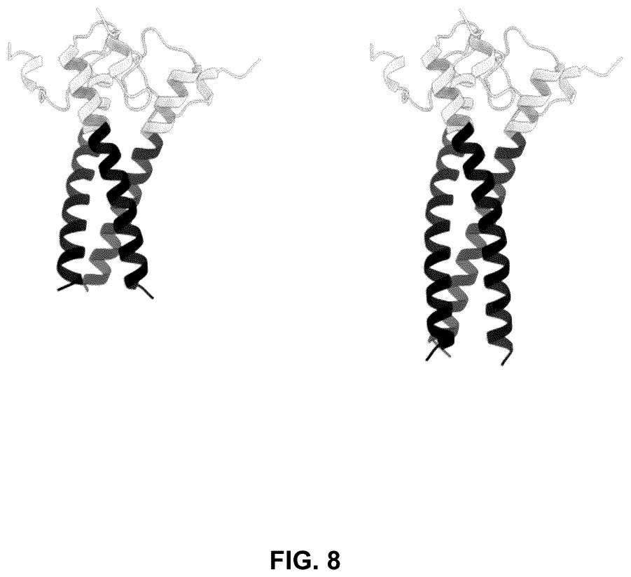

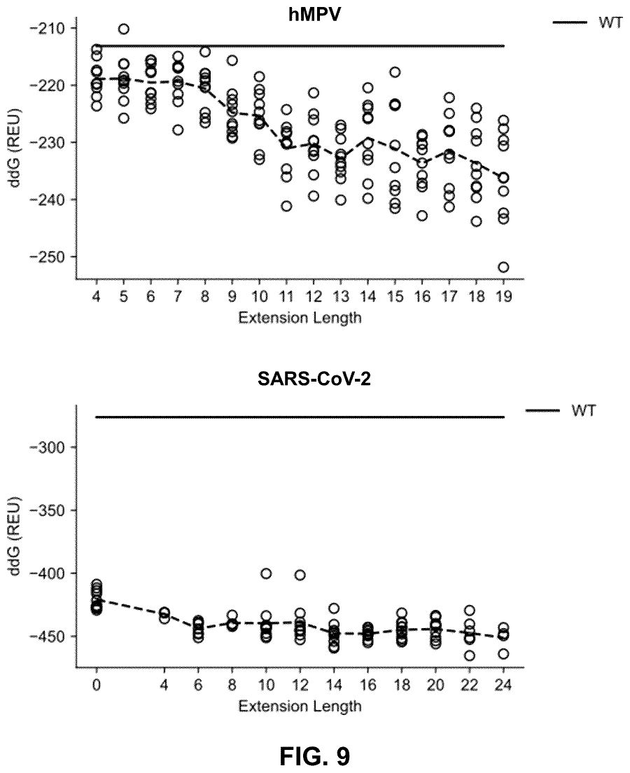

Remodeled C-terminal domains generally fell into two categories based on the geometry of the input structure. Where the input domain already consists of a relatively tight helical structure (for example FIGS. 6A-6D) the remodeled domain continues the helical bundle with straight or slightly twisted helical bundles with remodel lengths between 10 and 24 residues being optimal (FIG. 7). The input domain consists of converging alpha-helices, helices in the remodeled domain cross, with a well-packed hydrophobic core (FIG. 6E) RFdiffusion was also able to generate outputs where the helices converge into a tight helical bundle (FIG. 8). Optimal remodel lengths for these constructs were greater than 10 residues (FIG. 9). In some cases all remodeled lengths resulted in significantly better scores than the WT sequence (FIG. 9), in which case designs were selected based on their score relative to the average for that remodel length.

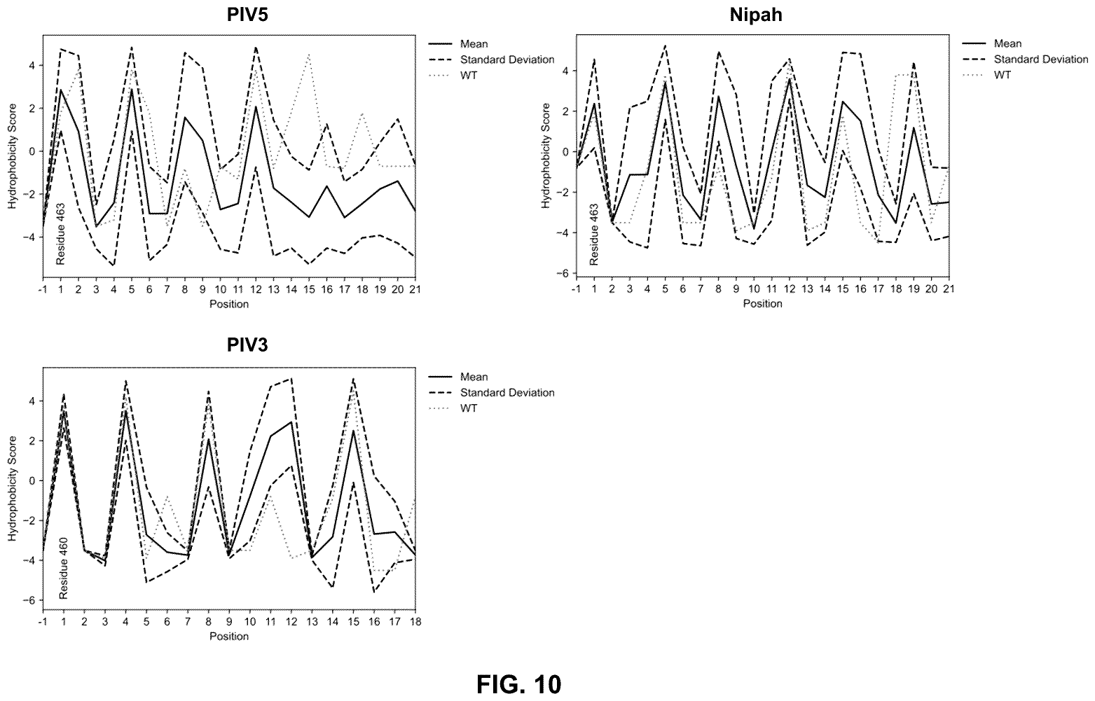

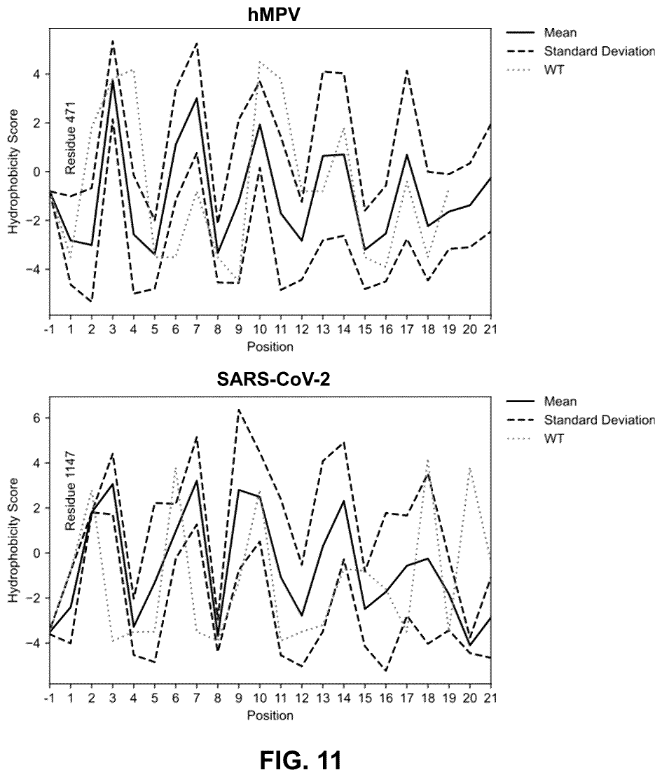

Selected remodeled sequences all result in helical bundles with repeating patterns of hydrophobic and hydrophilic residues. In most cases the WT sequence has a similar pattern, except that one of the repeats is much less hydrophobic than the remodeled sequences. For example, remodel position 8 is a serine in PIV5 and in remodeled designs is typically a leucine, isoleucine, valine, or alanine (FIG. 10). Designs with more distant C-terminal helices tended to result in designs where the pattern of polar and hydrophobic residues shifted relative to WT (FIG. 11).

PIV5: The input structure for PIV5 was 4GIP (Ref. 4). PIV5 has a glycan at position 457 which was preserved. The B-factors increase significantly from residue 460 to 464, so for that reason 459 and 460 were allowed to repack, and de novo sequences were generated for subsequent residues. 76 remodeled sequences were generated, ranging from six (6) to 26 residues in length. The designs generally improve hydrophobic packing, particularly at position 470 and 471. Some short remodeled sequences had excellent predicted ddG's but from the ddG plot the optimal length is ˜12-14 residues (FIG. 7).

PIV3: The input for PIV3 was 8DG8 (Ref. 5). There are no glycans in the PIV3 C-terminal helical bundle. The cryo-EM map quality deteriorates progressively along the length of the C-terminal helices and there is no side-chain resolved after residue 469. There is some sub-optimal packing at position 460, and so this position was allowed to design when using Rosetta remodel. Because residue 461 makes native contacts with the rest of the ectodomain its identity was preserved, and subsequent positions were allowed to design de novo. The residues after position 468 were removed. RFdiffusion does not allow for extension and partial diffusion simultaneously, so diffusion models start at residue 465. Ten (10) sequences were generated by Rosetta Remodel and 44 sequences by diffusion. The optimal length was 14-16 residues (FIG. 7). Therefore, remodeled lengths of 14 or more were selected for RFdiffusion.

Nipah: The input for Nipah was 7UP9 (Ref. 6). Nipah contains a glycan at residue 464 which was preserved in all designs. Because Nipah has a low-entropy methionine at residue 463, and no significant contacts with the rest of the ectodomain, remodel and diffusion both were allowed to design de novo sequences starting at residue 463. This required manual reversion of residues 464 and 466 to preserve the glycan. The optimum sequence length was ˜10 residues (FIG. 7), which was therefore used as a minimum remodel length for RFdiffusion. Fifty-three (53) sequences were selected.

HMPV: The input PDB for HMPV was 5WB0 (Ref. 7). The C-terminal resolution is much lower for HMPV than RSV. For that reason only positions 471 and 472 of the input structure are included in sequence design; all residues after 470 were allowed to design de novo. The optimum remodel length was 10 residues (FIG. 9) and the minimum remodel length for RFdiffusion was set at 10. Interestingly, the RFdiffusion pipeline struggled to generate well-predicted remodeled termini for HMPV. This is likely due to an interaction between the identities of the context provided for diffusion and ColabFold, and not an intrinsic property of the HMPV-F protein. As with RSV-F, HMPV-F remodeled designs tend to have a well packed hydrophobic core in three or four layers, starting at position 473.

RSV: A small set of C-terminal sequences were generated using RFdiffusion. Longer remodeled sequences up to 31 residues in length were well predicted. RSV designs are based off of 4MMU (Ref. 8).

SARS-COV-2: We selected 7LAB as the input structure based on a combination of reasonable quality data and good model building in the relevant regions (Ref. 9). Designs were selected based on the score relative to the average for that length (FIG. 9). De novo sequence design began at residue 1147. The optimal remodel length >10 residues, although some shorter designs with a remodel length of six (residues) formed very tightly packed helical bundles. For RFdiffusion, a minimum length of 10 was selected. Although the arrangement of polar and hydrophobic residues is largely the same for designs and the WT sequence (FIG. 11), the hydrophobic residues tend to be smaller, particularly at positions 1149 and 1153. This enables tighter packing, allowing residue 1150 or 1154 to also be hydrophobic.

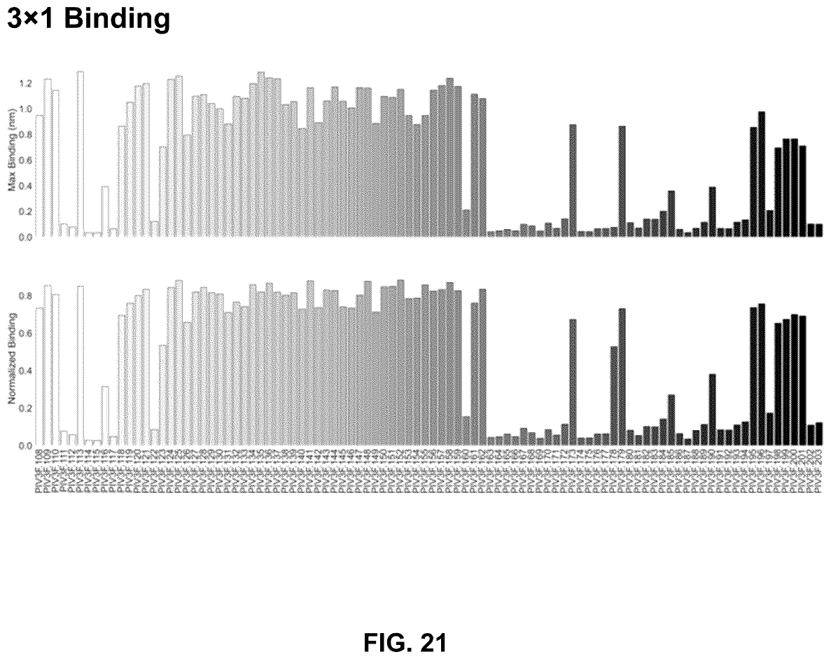

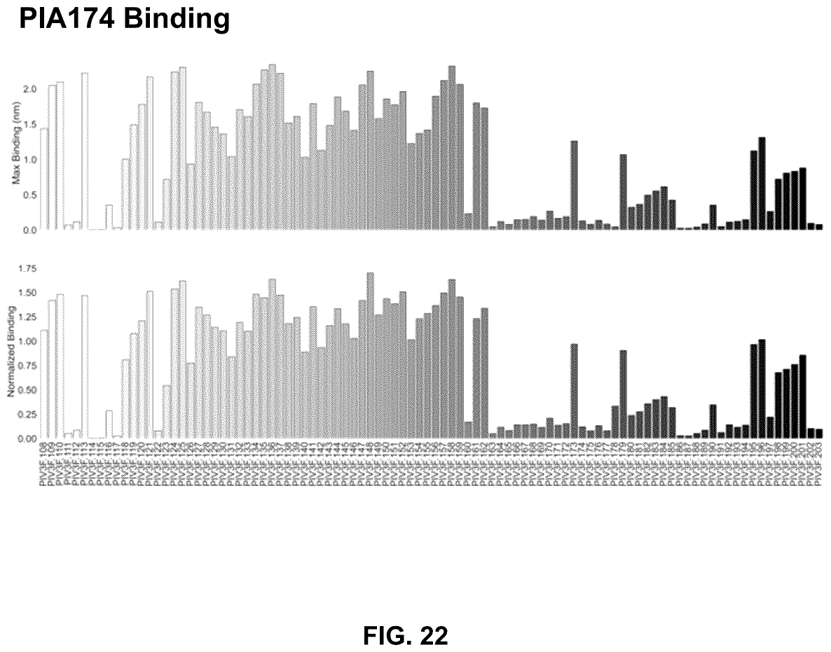

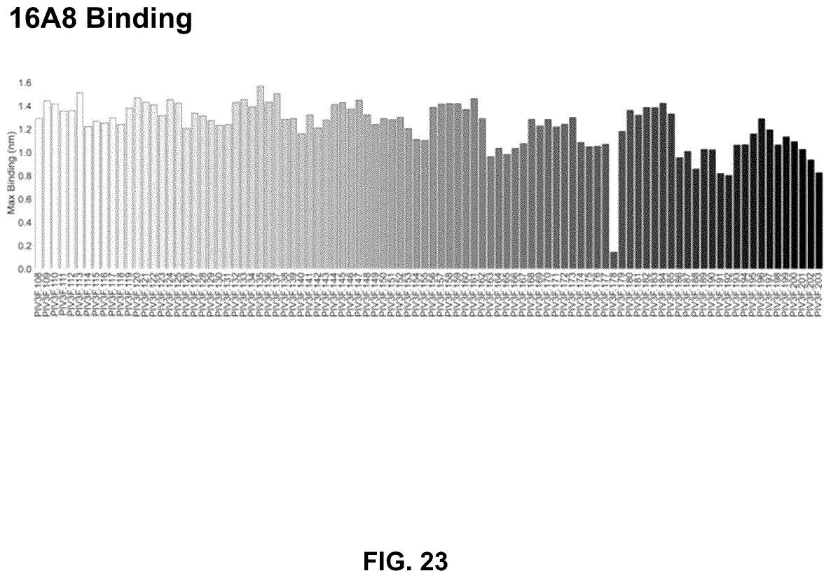

Experimental validation of C-terminal remodel designs in PIV3: The 53 C-terminal remodel designs described in Table 7B and Table 7D were genetically fused to I53-50AΔcys with a 12-residue Gly-Ser linker and expressed at small scale in HEK293 cells. These designs were compared against a control that uses GCN4 instead of C-terminal remodel designs (PIV3F.C) in addition to many designs that added novel stabilizing mutations in the F ectodomain relative to PIV3F.C (PIV3F.55-95, e.g., comprising SEQ ID NO: 716 to 756). The prefusion conformation was determined by binding to prefusion-specific monoclonal antibodies 3×1 (FIG. 21) and PIA174 (FIG. 22) using biolayer interferometry. Prefusion-specific monoclonal antibody binding was normalized to a CompA-specific monoclonal antibody, 16A8, to account for differences in expression levels (FIG. 23). 40 other, non-C-terminal remodel designs, attempting to stabilize the prefusion conformation are also included in the analysis. While only 8/40 non-C-terminal remodel designs are strongly prefusion, 36/53 C-terminal remodels are strongly prefusion and most have some 3×1 and PIA174 binding. Surprisingly, binding signals for 3×1 and PIA174 were higher for many C-terminal remodel designs relative to PIV3F.C, which demonstrates that this design technique can provide superior antigenicity and/or expression levels relative to genetic fusion to GCN4, which is commonly used in the field. Further, the success rate for this design strategy was far higher relative to designs that tested stabilizing mutations instead of the C-terminal remodel strategy.

The PIV3 fusion protein can be stabilized in the prefusion conformation by the addition of a trimerization domain such as GCN4 in addition to, and in between, the antigen and CompA (PIV3F.C in Table 22 and Table 23; comprising SEQ ID NO: 327). To better understand the effect of C-terminal remodel we expressed and purified three C-terminal remodel constructs in HEK293 or CHO cells. These three constructs (PIV3F.28, PIV3F.40, PIV3F.44, respectively comprising SEQ ID NO: 355, 367, and 371) were chosen based on higher levels of binding signal to 3×1 and PIA174 after small-scale expression. Purified yield was determined by UV-Vis, percent high molecular weight (HMW) species was determined by size exclusion Ultra-Performance Liquid Chromatography (UPLC), and prefusion conformation by antibody binding using BLI (Table 22). Thermodynamic properties were determined by nanoDSF, either using the extrensic dye SYPRO, or the intrinsic tryptophan fluorescence, and static light scattering to determine the aggregation onset temperature (T_{agg}). C-terminal remodel designs have modestly reduced % HMW species, and improved yield and prefusion antibody binding. Unlike with RSV, there were minimal changes in thermal stability metrics. However, WT PIV3 F protein has a higher intrinsic thermostability than RSV F.

TABLE 22
Characterization of WT and C-terminal remodeled PIV3 F constructs

| | HEK transient expression/CHO transient expression* | | | | | | | | |
|---|---|---|---|---|---|---|---|---|---|
| | | | | | SYPRO | SYPRO | ITF | ITF | T_{agg} |
| | % HMW | Yield | | | Tₒₙₛₑₜ | Tₘ | Tₘ | Tₒₙₛₑₜ | 266 nm |
| Construct | CompA | (mg/L) | PIA174** | 3×1** | (° C.) | (° C.) | (° C.) | (° C.) | (° C.) |
| PIV3F.C | 22.4/26.6 | 8.3/8.0 | 1.05/1.11 | 0.66/0.66 | 54/56 | 64/65 | 65/67 | 54/53 | 67/49 |
| SEQ ID NO: 327 | | | | | | | | | |
| PIV3F.28 | 14.3/9.8 | 29.3/16.6 | 1.30/1.33 | 0.73/0.72 | 55/58 | 64/65 | 65/67 | 54/51 | 67/67 |
| SEQ ID NO: 355 | | | | | | | | | |
| PIV3F.40 | 19.2/15.4 | 36.6/15.3 | 1.22/1.29 | 0.71/0.71 | 55/58 | 63/65 | 65/68 | 54/50 | 66/66 |
| SEQ ID NO: 367 | | | | | | | | | |
| PIV3F.44 | 17.2/15.3 | 39.3/7.9 | 1.28/1.31 | 0.73/0.72 | 56/58 | 64/65 | 66/67 | 53/51 | 67/65 |
| SEQ ID NO: 371 | | | | | | | | | |

*First value from HEK expression, second value from CHO
**PIA174 and 3×1 binding by BLI normalized to 16A8 binding

To further differentiate C-terminal remodel designs from the WT antigen, three selected designs were stored under stressed conditions at 25° C. or 45° C. for 30 or 14 days respectively. Stability was measured by size-exclusion ultra-performance liquid chromatography (SU-UPLC). The main peak area, corresponding to PIV3 F, and earlier eluting peaks corresponding to high molecular weight species (HMWS) were integrated and the percent-change relative to a sample stored at −80° C. was calculated. The designed constructs were more robust to stressed storage, as demonstrated by a 36.1% loss of main peak area and commensurate rise in high molecular weight species for the WT construct and only a 2-8% loss/rise for the C-terminal remodel constructs when stored at 25° C. for 30 days (Table 23).

TABLE 23
Stressed storage stability of WT and C-terminal remodeled
PIV3 F constructs

| | T30 @ 25° C. | | T14 @ 45° C. | |
|---|---|---|---|---|
| | Main Peak | HMWS | Main Peak | HMWS |
| ID | % Δ Area | % Δ Area | % Δ Area | % Δ Area |
| PIV3F.C | 36.1% | −36.1% | −68.8% | 68.7% |
| SEQ ID NO: 327 | | | | |
| PIV3F.28 | 2.2% | −2.2% | −42.6% | 42.6% |
| SEQ ID NO: 355 | | | | |
| PIV3F.40 | 8.3% | −8.3% | −52.2% | 52.2% |
| SEQ ID NO: 367 | | | | |
| PIV3F.44 | 1.5% | −1.5% | −51.0% | 51.0% |
| SEQ ID NO: 371 | | | | |

### Example 5. Consensus Sequence Analysis

Structures were analyzed by measuring the helical termini moment for two of the three protomers in the input trimer structures. The moment can be measured by determining the vector between the N-terminal alpha-carbon and an alpha-carbon near the C-terminus that is an integer number of helical turns after the first selected alpha-carbon. The dot-product between helical moments is a measure of helical orthogonality.

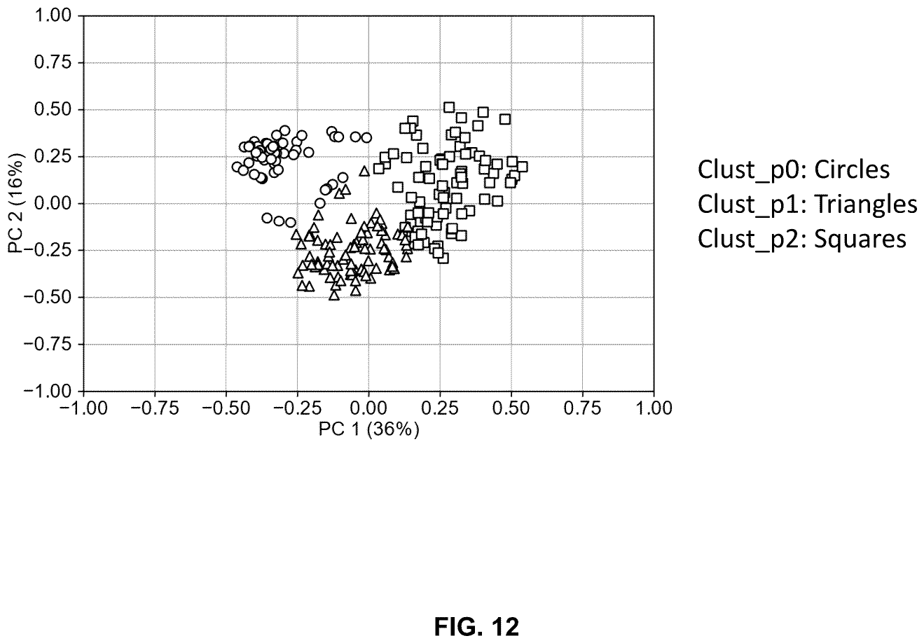

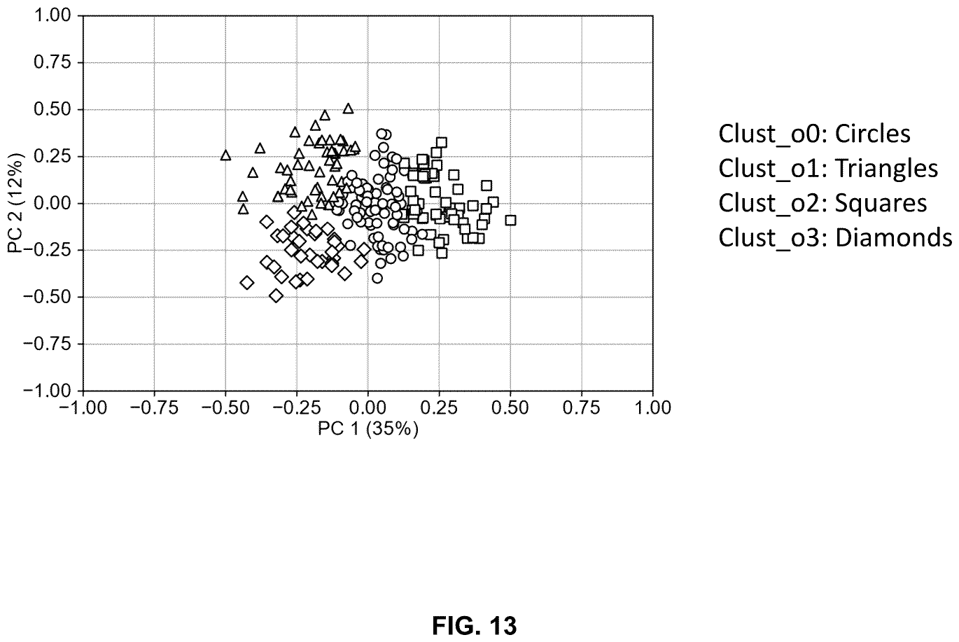

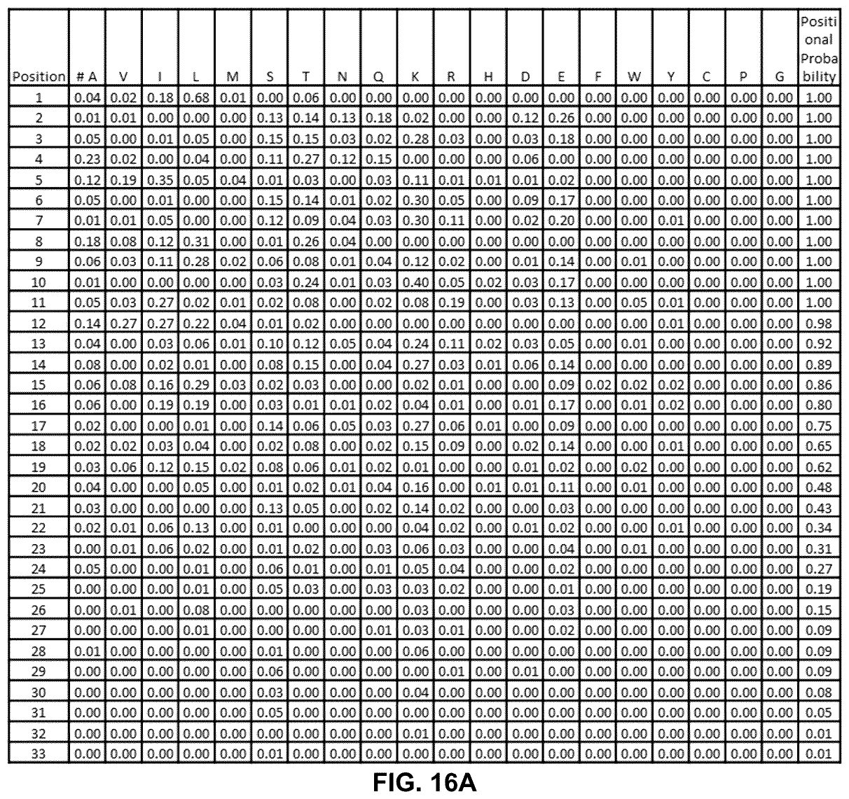

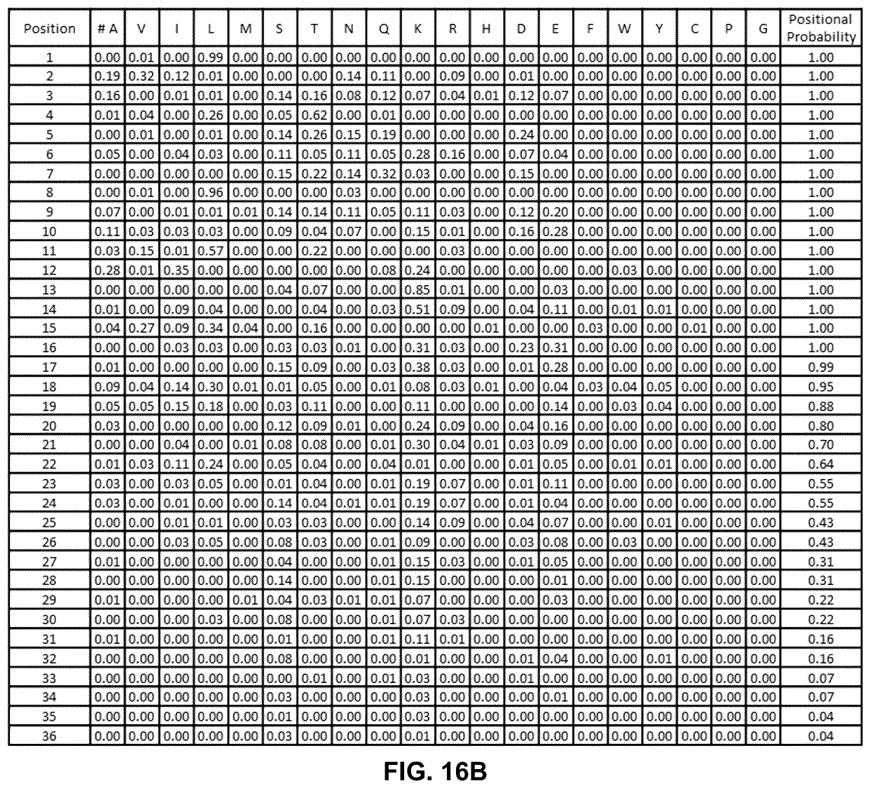

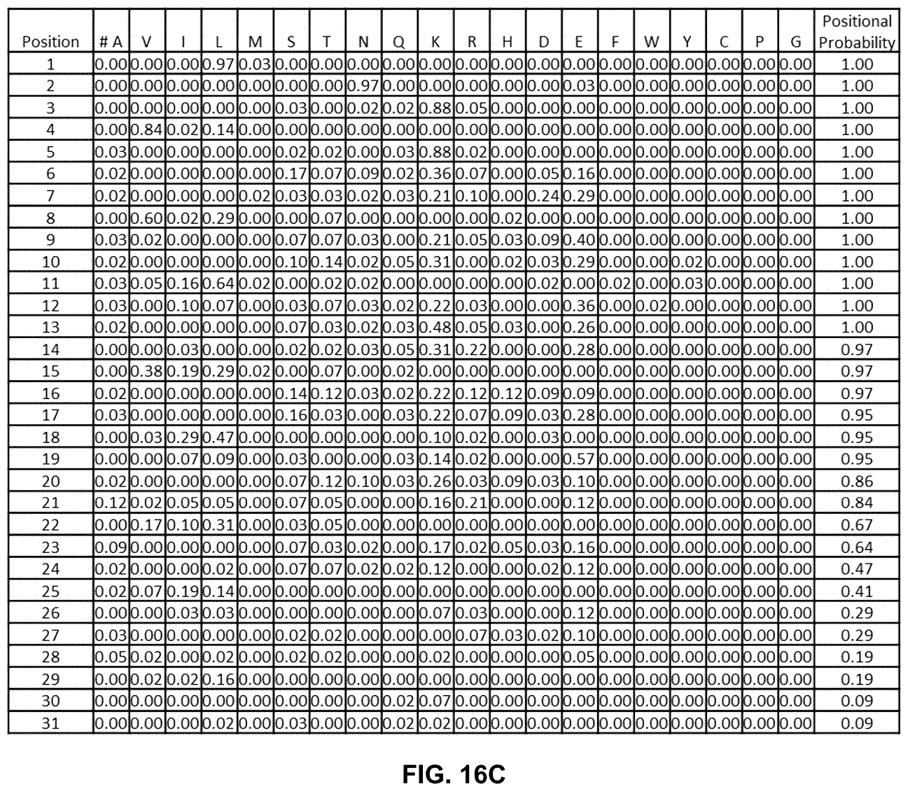

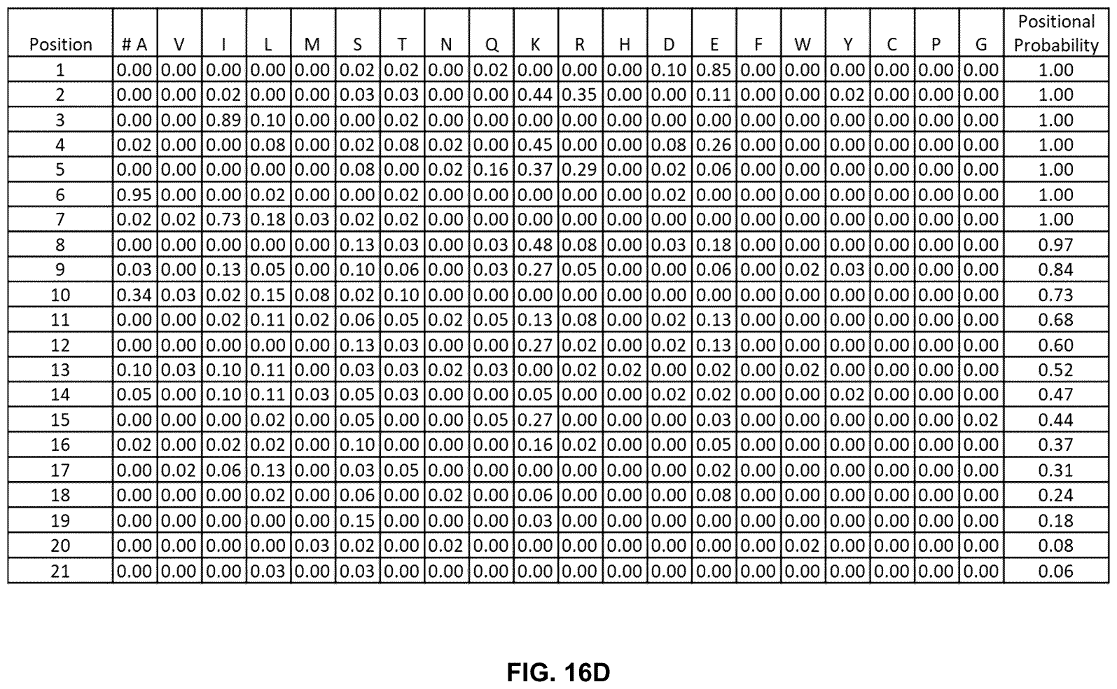

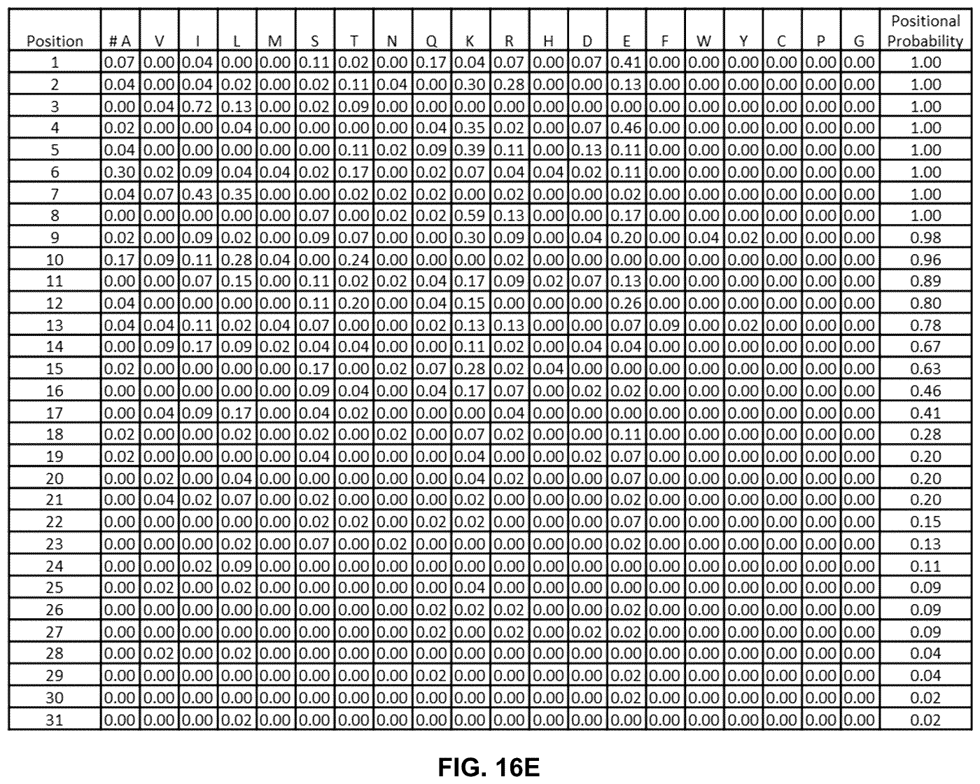

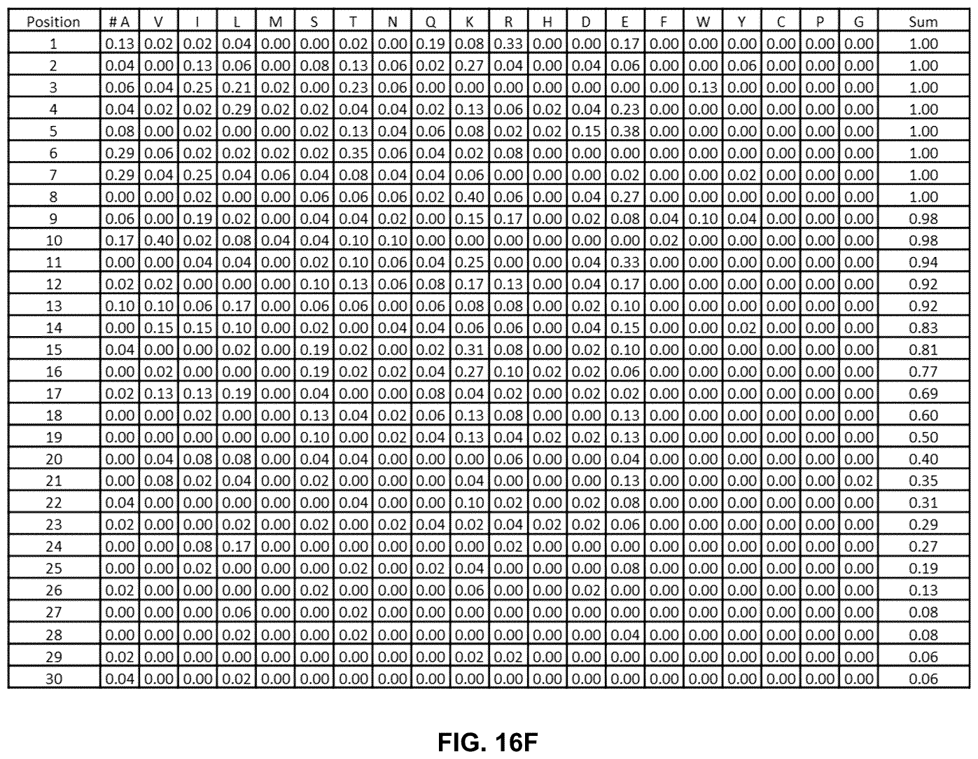

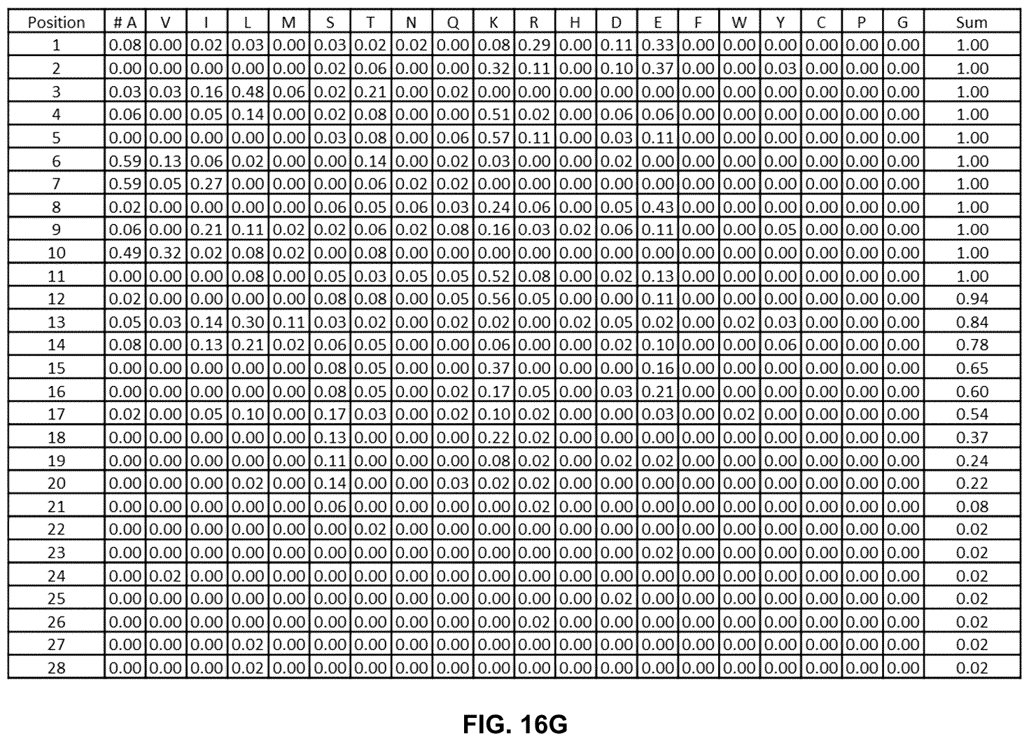

Consensus sequences were identified by first clustering input structures by C-terminal geometry. The dot-product of the C-terminal moments generally clustered into two groups with a mean of 0.92+/−0.03 and 0.77+/0 0.6, termed “parallel” and “not parallel” respectively. The former included Paramyxoviridae and Coronaviridae while the latter consisted of Pneumoviridae. Sequences derived from parallel helices and non-parallel helices were aligned respectively. Alignments were based on a structural alignment. For PIV5 the WT sequence LAAV ended up in the alignment, which would interfere with clustering. Therefore, MPNN was used to generate sequences to replace LAAV. Likewise preserved glycosylation sites would also interfere with the clustering. Glycosylation sites residues were randomly replaced with Q, N, D, S, or T to introduce noise at those positions in the alignment (position 1 in FIGS. 16A-16G). Aligned sequence distances were calculated using the BLOSUM62 scoring matrix and distances clustered using k-means clustering. The number of clusters was determined by inspection of the distribution of clusters in a principal component analysis (PCA) of the distance matrix. Three clusters were identified for the “parallel” group (FIG. 12), and four for the “not parallel” group (FIG. 13).

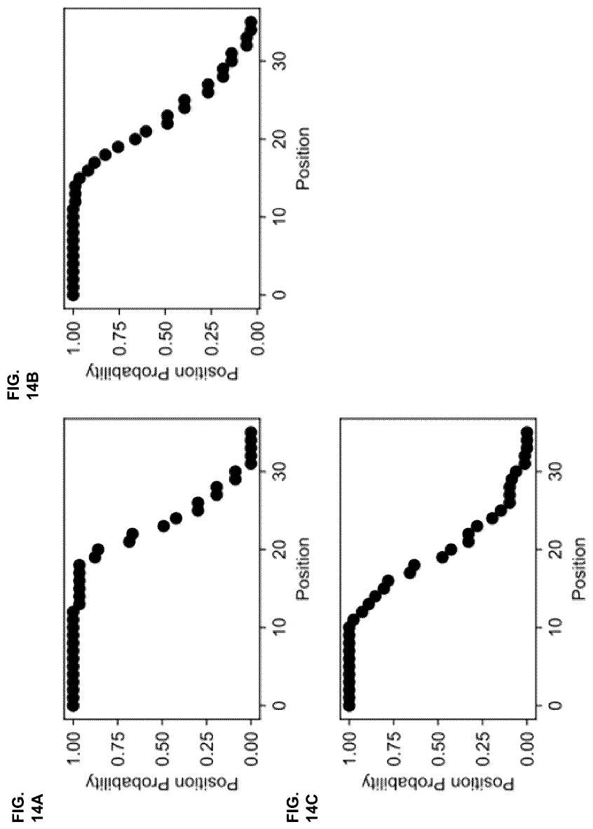

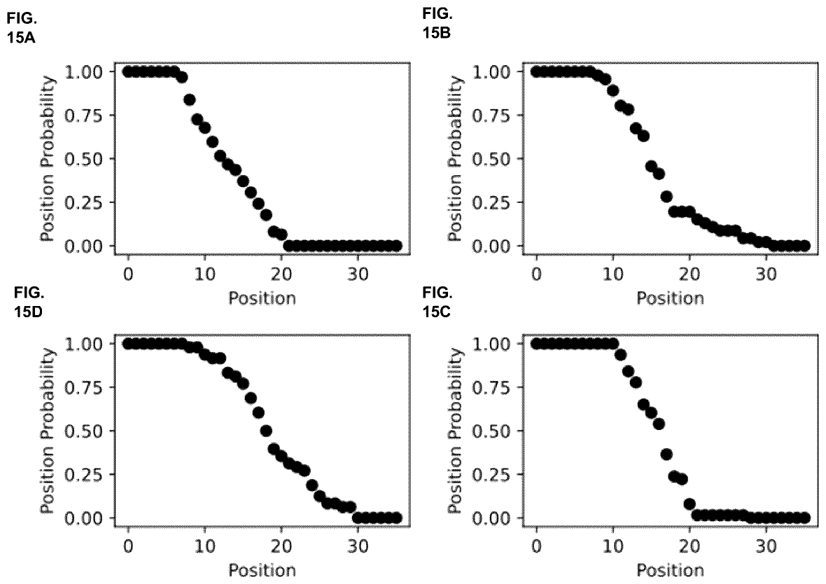

The consensus sequence for each cluster was calculated. Amino acid position specific identities and their probabilities were calculated. Because RosettaRemodel tends to prefer salt-bridges along and between helices, polar positions converged on lysine, for example EKIKKAIKKA(K/E)KLLKKL. Such a basic sequence is likely to pose challenges such as binding to biological polyanions and cell membranes. Furthermore, because the stabilizing effect is likely driven by hydrophobic packing, surface polar residues should generally be less critical. Therefore, unless a single polar residue was strongly preferred (no other identity was observed with >50% of the maximum position-specific probability), any polar residue is allowed at that position, specified with the letter X₂. Likewise hydrophobic positions that do not strongly favor a single apolar residue are specified with X₁. Table 24 shows the consensus sequences for each cluster. The length of the C-terminal remodel is determined from the sum of the position probabilities which decay at a characteristic length defined here as the length where the probability falls below 50% (FIGS. 14-15, Table 24).

TABLE 24
Illustrative consensus sequences and weights

| Termini | | | | |
|---|---|---|---|---|
| Orientation | | | | SEQ |
| (dot product) | Name | Consensus Sequence | Length | ID NO: |
| > 0.85 | Clust_p0 | LX₂X₂TIX₂X₂LLX₂I[V/I]X₂X₂L | 19 | 573 |
| | | [I/L]X₂X₂L | | |
| | Clust_p1 | LV[A/T]TX₂K_{X2}LX₂DLIX₂X₂L | 24 | 574 |
| | | [K/E]X₂LLX₂KLX₂X₂ | | |
| | Clust_p2 | LNKVKKX₂VX₂X₂LX₂X₂X₂V | 23 | 575 |
| | | X₂X₂LEKX₂LX₂ | | |
| < 0.85 | Clust_00 | EKIX₂X₂AIKKAX₂KL | 13 | 576 |
| | Clust_o1 | EX₂IX₂KAIKX₂L[L/X₂]X₂X₂ | 15 | 577 |
| | | [X₁/X₂]X₂ | | |
| | Clust_02 | X₂K[X₁/T][L/E]E[T/A]X₁X₂[I/X₂] | 19 | 578 |
| | | VX₂X₂[X₁/X₂][X₁/X₂]X₂X₂X₁X₂X₂ | | |
| | Clust_03 | X₂X₂LKKAAX₂IX₁KKX₁LK | 17 | 579 |
| | | X₂X₂ | | |

X₁: Apolar residues AILM
X₂: Polar and charged residues STNQEDRKH, WT preferred if within the polar set.
[A/B]: A choice between A or B

TABLE 25A
Illustrative consensus sequences of “parallel”″ groups

| | | | |
|---|---|---|---|
| | | | SEQ ID NOs (left |
| Sequence | Sequence | Sequence | to right) |

Cluster 0

| | | | |
|---|---|---|---|
| LQQNISSLEKALKKAE | LESAMKTAMKIIS | LQRTVDKLNSQIQALI | 757, 758, 759 |
| KDLEEVRRQL | | | |
| LSKNVESLAKEVKKL | LKKAMETAIKRINKA | LTANASENTARIEALER | 760, 761, 762 |
| EQKLNSL | | RIHELEL | |
| LSQTIKNLQDEVTKVT | LEKAAKKTLKIAKEES | LTENVTNLKKRLSEVE | 763, 764, 765 |
| EELKKLVEQL | TKDKS | KVIKTL | |
| VNTTVRKLSEILAS | LEKAIKKTLKIIRTELSI | LDNNITSLSERIHKLEN | 766, 767, 768 |
| | S | L | |
| LSKNIEEIEKRLSELES | LESAIKKALTIIKQIWS | IQESLQRLSERVEEIER | 769, 770, 771 |
| TIKKL | | R | |
| LDSDAESLADKVTAL | LDSAASRALKIAIELL | LNTQVKKLKDRIKKIE | 772, 773, 774 |
| ETRIKSIEA | RATESKK | ERLN | |
| LQKDVKSVETRLRT | LEKAASKAIKISLKILK | LSSNVSNLRTDLNDLK | 775, 776, 777 |
| | EILS | KLVKKLIELL | |
| IQTNIKQNTERIDKIEK | LEKAIKEALKR | IDKDIQKNTERINKIEK | 778, 779, 780 |
| TLK | | TIKSLIS | |
| LQRDVRKLEKRLTHV | LETAIKIALEIARKEIS | ISENLKEAQERVDKIEK | 781, 782, 783 |
| EEVLK | | LLEKILR | |
| IDKSIKSLDTRL | LDSAASYAIKV | LDSDITAIQETL | 784, 785, 786 |
| IDKSVDSLLTEVHAIR | LEKAAKTALKIAS | LQKQIKELRTVVKRLL | 787, 788, 789 |
| HEIDQLRS | | | |
| LNTDVKQLQTSL | LEKAAEEAVRRAIKL | LTRNIKDVKQAL | 790, 791, 792 |
| | YKENLKKS | | |
| INENISTITTEIKKIKEIL | LETAASIAEKIARKLL | ISSNITELKKTL | 793, 794, 795 |
| L | KES | | |
| LQDQISKLSNRVQRLE | LESAIKKTLKIISKRNK | IQENMERTKKWITKLI | 796, 797, 798 |
| RRLQEIERRL | DS | AKWKS | |
| LQEDVERLETLVREV | LEKAIKKATEIARKLIS | ASKDMAEIIKTIKSLLK | 799, 800, 801 |
| QKQLE | | KS | |
| LNEQIESIEKDIAT | LESAADKTMKKYKTE | ATLDIEKTKRIMTSIAL | 802, 803, 804 |
| | AKRS | YVWTLIAKELKSKS | |
| LNKDLDELSSQLADLS | LETALRIAIEITLQLLK | IQETIKKVKKTAAEAIT | 805, 806, 807 |
| ARVEALQSTL | KMAS | TQTRIWQKLKKSKSKS | |
| LDNSIKDLAKRVSDIE | LEKAIKITLKIIDIKLS | LSEDIDKLEKKMSTIAK | 808, 809, 810 |
| SLVQKLLS | | KLSKIEASKRKSSS | |
| IDSSISRNTDKIKELQQ | LEKAAKKALEIASRS | TNINVTKTEKKVEDLL | 811, 812, 813 |
| EIEKLQSSL | | KKLTS | |
| IQENVKKIEEILRSMS | LSKTKAETLETVREL | IDESVTRLAKILKKLI | 814, 815, 816 |
| AQLTIETLARIVSTWY | LEKTQSTTLTAAKTLI | LETTRTKTITEVNTTIST | 817, 818, 819 |
| KQQAKKTATEEKRKS | KST | T | |
| MNTQIDQIEKWLRDK | LETTKKETLTEVTEA | LEAVKTETLTAATTAI | 820, 821, 822 |
| EKKEQS | | NSALAKQ | |
| IDESTKKVKKIALDIAS | LESTKAVTETEIKAEIN | LKETQEKTITEVIKILN | 823, 824, 825 |
| INESLKSLATDVKKLK | LNTTKTETISSIKKEIE | LTNTENNVLTRVKQS | 826, 827, 828 |
| SKI | TM | | |
| IDEDIDSLKKEVKKYI | LETAIKITLEIVLKILKE | LNALETRVLTAIN | 829, 830, 831 |
| EKAEKDKKS | WEKRKSS | | |
| LDDTVRKALKWIKEV | LEKAIKKTLKIIWTELS | LTKLKEEVLEEVETMI | 832, 833, 834 |
| KKKS | IS | RETAA | |
| LNEDIIKILQKLLTWIT | LVSTNAQLVKTIKLVI | LDATSSRAIERVTTLLE | 835, 836, 837 |
| KTKQEKKS | KAILTAIKEKKASS | | |
| ANLQIEKTKRKMTSIA | LADSSRDLSHVIQIML | LDKVKDETVTIMTKYI | 838, 839, 840 |
| KEVKTRIAKEEKSKS | ETLETATKQKKKDS | QET | |
| TNLTVEKIWRYLMAV | LQTLKEESTHLTKTLL | TQSQTEKILQWIKKFET | 841, 842, 843 |
| LS | S | KVKS | |
| TTKNTATIEKIVRSLL | LEATHTRTLTTVTAA | TTLTVTETIKELKSTDK | 844, 845, 846 |
| KEIKSERTR | | KLKKYIKTVQSS | |
| IQEDVTRLKKIVEKLIR | LDTTKKETLTEAQETL | VNKLKSELKTWIKQEA | 847, 848, 849 |
| ELQKIK | ERA | NEKA | |
| TDTDVSKTLKMLLEFI | | | 850 |
| TREERSKR | | | |

Cluster 1

| | | | |
|---|---|---|---|
| LVSSSKDLSEVIKWVR | LAETDATLQEVAKKL | LRATTTNLSELAKELK | 851, 852, 853 |
| EVVSKWIS | EEKIRTDIKREQS | KLKEHILRYQ | |
| LVQTNKTLDDTIKKLE | LTDNLDNLEERVKRL | LVNTTSDLSETQKKTK | 854, 855, 856 |
| KLERELRSRWDSERK | EEEVKKLKE | ETATKLEQKTEKTLKY | |
| S | | TKKK | |
| LIDTSKDLESLKKKLD | MNRLKKKLDQLWKIL | LQATSDSLIKTQKLLKE | 857, 858, 859 |
| ELTKKS | KEDKDKS | LI | |
| LQSTQKTLDALKKKV | VNKTQKKLKEIWKKL | LVATDRSLSALAEKCK | 860, 861, 862 |
| DKK | KKELTKERNTLKS | KLKKKLEEDLKS | |
| LIKLSNSNTATIKKLD | LIATSKSLETTISILEEF | LRQTTDQLNSVIKILKE | 863, 864, 865 |
| KLVKS | LRRYKKKE | IKEMLDKLLEKSKKS | |
| LISTNRNLAELAKKLD | LNDLSKDLEVAIKKID | LVSSNSSLQELIKKVIT | 866, 867, 868 |
| KTIEKASKDDSKKS | KLES | LEKKS | |
| LRQTQSQLAKTQKLV | LATTNRQLEELAKKF | LQDVQSNLEKLIKEVK | 896, 870, 871 |
| TEILEKLTK | KEAS | S | |
| LANTSKSLRIVIKEIRK | LQQLNLTLTELKKRTI | LQELTDDLAKLASKVE | 872, 873, 874 |
| LKS | KWYEETLKRT | TETRKERTKKKS | |
| LVDLSSQLKSLWKIM | LVDTDKDLEDTIKKLE | LVQLQKTNEALIKAITK | 875, 876, 877 |
| EKLS | ELTTK | KEEKSTRKERSERKS | |
| LVATQSNLRNVIKIIES | LRKTNIDLTTLATKVE | LATTQKSLLETIKKVD | 878, 879, 880 |
| QTRS | KALS | KLTS | |
| LATTDEDLAALQTDIK | LVTTSNDLTSVIKKLD | LAATQNQLTELKKTTE | 881, 882, 883 |
| RLKS | KIVKKLQS | KVIRTLKTKEEKKKQE | |
| | | KS | |
| LNKLDRSLDKVKKKV | LIKLSSNLMDLARKTK | LATTTDNLTALKKEHE | 884, 885, 886 |
| DKAITEIKS | EYWEKEERSKKS | ELLKEIKKEKEEKSRS | |
| LASSNQDLTELAKIVK | LVDTSRNLEELAKKA | LLTTDKQLKELKKETE | 887, 888, 889 |
| SLIS | KKFTEKLLSEIKKTKS | KLKKKV | |
| | D | | |
| LRSTSRNLNNAIKRVL | LAQTDKNLEKLATKT | LVDLQQNLEELAKEVK | 890, 891, 892 |
| SWYKKKADEESS | KQLEEKLEKEKKKSS | KK | |
| LQALTKQLTDLKKKL | LVNLQTSLKDLKKKV | LVSQNLQLNKLAKRV | 893, 894, 895 |
| DSILTEQKRRS | DSK | KKYWEEVKSRS | |
| LNNLDRNLNNLKKKT | LILTTNTLNNTITIMKK | LNDLTKNLSKTQKLLK | 896, 897, 898 |
| EEIATDLEKKWRKMS | IEEKLKADKKKSS | ELI | |
| KS | | | |
| LAATTAQLTKTIKEM | LQATTRDLDDLKKKV | LNQVDRSLKELESELK | 899, 900, 901 |
| KEK | DTLEKQS | SRLS | |
| LNALSTDVDDVIKKL | LRTVDSNLNSLAKKL | LVTTDQQLTSLAKQTK | 902, 903, 904 |
| DEALSRI | DS | KLEDELRS | |
| LVRTTQDLEDLAKRT | LARTNNDLEALAKYV | LVITQRTLDDVAKRAE | 905, 906, 907 |
| KTWYDILAKILASNQ | S | STIRDLKETKKKQKKE | |
| KS | | KS | |
| LQNVQNNLNTLKTKI | LVHTTESLKLLKKRLE | LRQLNATLSETIKELKS | 908, 909, 910 |
| EQILKS | DYIKTQKAKS | HLTTLKIEKSKKS | |
| LVTTTNNLKKTAKIAL | LNELDANLQATIKTTE | LNSLDRTLDNLKKKVD | 911, 912, 913 |
| TVEKILTTRDKQKKK | KALKIILKRIKKALAE | EATKTT | |
| KDEKS | QKSS | | |
| LVTTSRNLDVLASDVS | LVSSQIDLDDLIKKTD | LIELNNDLEELKKKLEE | 914, 915, 916 |
| SMKATEEKKS | ALEKS | ILASIEKKEKS | |
| LVATQTNLALVIKKV | LIATNKNLSKLKKKLE | LVRTQESLNELKEKLD | 917, 918, 919 |
| ETIASKLKS | KIL | RYI | |
| LIQLSRDLSDLKKTLE | LASTNKSLSILAKKTK | LVTTDKTLQETQKQLE | 920, 921, 922 |
| KR | EAIDRIRS | TLAKKIKS | |
| LAETSKNLKSLIKKEN | LAQTSKTLSETIKKVD | LNNATIQLERVIKDLK | 923, 924, 925 |
| S | KSTKSTEKKS | KTKEKQKRSS | |

Cluster 2

| | | | |
|---|---|---|---|
| LNKVKEDIEKLEERVH | LNKVKERVKENEKIIT | LNKLAKEVKTILKKLS | 926, 927, 928 |
| AIEKK | KIQKTLD | KKLSSLES | |
| LNKVKNRVEKLEETL | LNKVKTEVKEITKKV | LNKVKSKTETMAEKM | 929, 930, 931 |
| TRLINA | RELEERLRKVEEVVKS | RSKETATS | |
| LNKVKDDLESVNKRV | LNKVKSDVRDLEERL | LNKVKSKTETYIKETRS | 932, 933, 934 |
| SEIEHELHEIKA | HKLETRLEEI | KETATS | |
| LNKVKEEVKELTEEIH | LNKVKSEVKKLKERL | MNRLKSKLDKLLKELK | 935, 936, 937 |
| ELREEVEALKEEL | EELEAR | EDKDKS | |
| LNKVKQQVEKLIERL | LNKVKEKVDKIQENID | LNKVKKETKTFIKEVR | 938, 939, 940 |
| HRLENKLAEA | AIKTILD | SKETATS | |
| LNKVKTELHKLKERV | LNKVKNEVSELEKRT | LNKVKSKTETYIKEVR | 941, 942, 943 |
| RDIEKKLA | TKIESTIKTLIE | SKETA | |
| LNKVKKEVEELRKRL | LNKVKDKVEKDTKKI | LNSLQRDHEKLIKEVK | 944, 945, 946 |
| KKLEEKLTSV | KEIEHELA | | |
| LNKVKKKVSELEKQV | LNKVKKDLKELSEKV | LNSLQKSLVELKKKLD | 947, 948, 949 |
| TEIEKILTEIRA | HELLNS | ELEKR | |
| LNKVKERLHKLEESV | LNKVKKRLEELEEKL | LNKLNRQLAALAKKT | 950, 951, 952 |
| KQLKKA | DRLEHIVHLL | KELEKKIKS | |
| LNKVKSDVENLKEKI | LNKVKENVEEIEHKV | LENLKNTVESIIN | 953, 954, 955 |
| NKII | KEIE | | |
| LNKVKDDVRTIKKEL | LNKVKKEVNELNKRI | LERIRTEVTQASA | 956, 957, 958 |
| EELKQLVKNL | RSLEQRVEKLERALK | | |
| | K | | |
| LNKVKERVKSLEKQL | LNKVKKDLKKTKENL | LNKVKKDVTYLKTEV | 959, 960, 961 |
| KTLL | KEVEEKVKELLS | AQLQ | |
| LNKVKTR VEEIERKIS | LNKVKKELEELLQKV | LNKVKKEVKELKERLD | 962, 963, 964 |
| SLEKEVEDIRRSLQQ | KDLEEKVETL | HVEKRLKEVEEKL | |
| LNKVKNKLEKVESQV | LNKVKKMVESLESKV | LNKVKEDVASLKKEVE | 965, 966, 967 |
| HRLENRIEKIERLLKS | TKLEKTVKELLT | KIIKA | |
| LNKVKRDVEQLRQEL | LNKVKSELDKLKKKV | LNKVKNSLDKVEKKV | 968, 969, 970 |
| NSLSKRVHKIEEAL | EHIENS | TSLI | |
| LNKVKSAVTHLTKEV | LNKVKKDVEKLKKRI | LNKVKKKVESLERKVS | 971, 972, 973 |
| TKLKEL | SHIEKLLS | KLENEIKTIID | |
| LNKVKKDLNDAKKRI | LNKVKKEVRKLEHEI | LNKVKKKVSELEKRV | 974, 975, 976 |
| SHIEKVLN | HEIKKRLA | DHIEHRLKQI | |
| LNKVKADLTTLESKQ | LNKLAKEVKTILKELS | LNKVKKKVEKIEKEIE | 977, 978, 979 |
| SEIERRVAKIEHAL | KKLSSLES | KLKRELETVKREI | |
| LNKVKEEVEKLERET | LNKVKSEVSELKTKV | | 980 ,981 |
| KKLSHEIKKIKETL | QTLETRIKKIEHELKL | | |

TABLE 25B
Illustrative consensus sequences of “not parallel” group

| | | | |
|---|---|---|---|
| | | | SEQ ID NOs (left |
| Sequence | Sequence | Sequence | to right) |

Cluster 0

| | | | |
|---|---|---|---|
| DRIKRAL | ERLEKALQTLTKAMKK | EKIERAIRKLES | 982, 983, 984 |
| | TLS | | |
| ERIDKAIS | TKIEKAITS | ERIDSAIKKALS | 985, 986, 987 |
| EEIEKAIKILKKILKES | EEIKKAIKILKKILKELSS | EKLKRATEKARKS | 988, 989, 990 |
| | S | | |
| ERIKKAIKTAIEAMQKS | ERIKKAIEIMLSWKKAL | ETILRAIKKAQKS | 991, 992, 993 |
| | EKNS | | |
| EKIEKILKELEKEKQSR | DRIERASKS | EKLAQAVS | 994, 995, 996 |
| EYIEKAIKAAQETIKKL | EKITKAIKIAKELKKLIES | EEIKRAIEALRKR | 997, 998, 999 |
| | ML | | |
| ERIEKILKELEKEKQSR | EKITKAIKIAKELLKKIES | ERTEKAIKITLTIS | 1000, 1001, 1002 |
| | ML | | |
| EIIKQAIS | EKLKKAIEQMLTVKKIT | EKITKAIEEMKKQ | 1003, 1004, 1005 |
| | EKWS | S | |
| EAIERAIKDMLTAKKQS | ERIDEAIKR | EKLEKAMEETKK | 1006, 1007, 1008 |
| | | LS | |
| EEILRAIKTARTESKKT | QKILDAIKS | ERIKSAIKKLESQE | 1009, 1010, 1011 |
| | | S | |
| EKIKKAIEKAESIIQSIS | ERIESAIKS | EKIKSALELALRL | 1012, 1013, 1014 |
| | | AK | |
| EEIDKAIKILKKILKELS | ERITKALQS | ERIERAIR | 1015, 1016, 1017 |
| EKTKKAIKITEEIYKKLS | ERIEEAIRR | ERIEEAIRRASKND | 1018, 1019, 1020 |
| | | G | |
| ERIKKAIKTANEHLSKVN | DRIKKALSKL | EKIKQAIELTLKLA | 1021, 1022, 1023 |
| | | S | |
| EKIERAIKWIEDLLKKEK | DKIKRAITKT | DKIKRAIS | 1024, 1025, 1026 |
| S | | | |
| EEIKKAIKEARKAIEKLK | ESIKEAIKQS | EKIKRAIDIVEKLT | 1027, 1028, 1029 |
| S | | QS | |
| EEIDKAIKEARKAIEKLK | EKIKQTMKKAS | ESIERAIKSTKEAI | 1030, 1031, 1032 |
| S | | KS | |
| EKISQAIDKTTKIILSIES | EKLTQAAS | ERIKRALEKLTKA | 1033, 1034, 1035 |
| | | TKS | |
| ERIKQAIKKVEETLKRLK | EKILQAIRLAS | EKIKQAIEYMLKV | 1036, 1037, 1038 |
| S | | AKS | |
| DRIKRALS | TKIAEAIKRTS | EKIERAIKKASS | 1039, 1040, 1041 |
| ERIKNAIKKME | ERINQALKKAD | EKIERAIKYALS | 1042, 1043, 1044 |
| EKIERAIKKAQS | ERILSALS | | 1045, 1046 |

Cluster 1

| | | | |
|---|---|---|---|
| QKIQDAVEELQTLMQKL | DRSERAQK | EEIKKETKRIRS | 1047, 1048, 1049 |
| EELKKAASKAKEEIKRS | DKASKAIEYAERDAKSK | EKMTKKANTAES | 1050, 1051, 1052 |
| | S | | |
| EEIKTIISILKELEKRS | SEIKKVITETRKITKKIKS | EKMTKKANDAES | 1053, 1054, 1055 |
| | S | | |
| ETLKKQASKAEELEKRS | DKLTRTAQKAKTLIEET | EEIDTLAKELKES | 1056, 1057, 1058 |
| | KKS | | |
| SRLKAELKKLKEILKKS | DKLTRIAQKALTLIEETK | IKIKTAAKQAKKK | 1059, 1060, 1061 |
| | KS | | |
| EETKQAIKLVKKDYKEK | SKIETAIKKLIEKERKTR | ERIKETNKATKQK | 1062, 1063, 1064 |
| S | AKK | | |
| EIIKQEIKKTQTFIKKVS | ERIKKTAKIAQKLYKTL | AKIETAIRKTIES | 1065, 1066, 1067 |
| | KSQS | | |
| ETIKREIKKTREMTKKLL | ERIDKTAKIAQKLYKTL | SRIKAMIKKILKS | 1068, 1069, 1070 |
| | KSQS | | |
| SRLKKAADKAS | ETIEKKLQS | ERLKKAAEIVERQ | 1071, 1072, 1073 |
| | | T | |
| ERLDKDAKTAK | SKIKKDL | ETIKKIIEEILSRS | 1074, 1075, 1076 |
| DKLKRTAEKAKS | ERLERHLRSR | ETLEKVAKEVTKI | 1077, 1078, 1079 |
| | | S | |
| EEIKTLAKELKE | IRTKQAIKSA | DELKRVITDLRKL | 1080, 1081, 1082 |
| | | K | |
| ESSKKAQKQAKS | SRIKKILSEAS | EKILTAIKIALAAV | 1083, 1084, 1085 |
| | | S | |
| DRLIKVAEKTSKMLKS | ETIKKLLKKAM | ERLDKTAKETKEY | 1086, 1087, 1088 |
| | | LS | |
| DRLKKMLEKTSKMLKS | EKIKQIARLAS | DKIKKAVSWVLA | 1089, 1090, 1091 |
| | | VKS | |
| ETIEKKLKTIESRLKS | EKIEQTRRLAS | EKLEKLERKTRQK | 1092, 1093, 1094 |
| | | DS | |
| ETTKKAIELLKKLYKS | REIETAIKKAKEFIKTIK | EAIERTLKTIDKKV | 1095, 1096, 1097 |
| | | S | |
| EDLKKTAAEAKKHIKS | RKTEEALRRADTIIKQLA | EELKKVAKEAKK | 1098, 1099, 1100 |
| | SKS | AIS | |
| ETIKKHIEIAIKFIKEV | AETKKAIERAREL | AKIEKTLKKLKTE | 1101, 1102, 1103 |
| | | DS | |
| NTVRKTIETVNSLEKELK | KEAKKAIETAKKLS | ARIKKTIEIVLTQT | 1104, 1105, 1106 |
| ELRTEVDRLL | | S | |
| KEIRNTVKKVRTIEKRLN | REIKDAIKKAKEFIKTIK | REVKEAIKIIKKIL | 1107, 1108, 1109 |
| KLETSL | | KKQS | |
| KLVKKVIKETHEIKKKLEDLLK | | | 1110 |

Cluster 2

| | | | |
|---|---|---|---|
| QTTEEQIKTLTERVESIEK | QKILDEIKKT | IRWEANAKKAETE | 1111, 1112, 1113 |
| EG | | IKKLSES | |
| QEIDKKLEYLEERVHDLE | ETILTTNKRAN | EITDRKNKKA | 1114, 1115, 1116 |
| ERLESLVQQLQ | | | |
| QNVEDRLEANEKAISHIE | QIIQDTIKKMS | EIAKQLMTKA | 1117, 1118, 1119 |
| QLIDQLI | | | |
| QNIEDRVEDNDDKVAEL | IKIKQQIKRLDEK | RAIKETQKRTTVL | 1120, 1121, 1122 |
| KEELEAIK | | EEDLKRVKELLKS | |
| QNVEDRLEELESRIKKIE | EYLLAVAETLNRR | RKATETIKKFEESE | 1123, 1124, 1125 |
| EEIEEIKKD | | KS | |
| QNIEEDLESLKERIHRLES | EYILTAIKIMLTR | RKWNESSKKVQE | 1126, 1127, 1128 |
| EVQNLLER | | QDS | |
| QRTEKRINDLESRVARIE | EILTQQAS | RRTLTAITRVERK | 1129, 1130, 1131 |
| EVLSL | | DS | |
| QETEDTLESLSQEVEKLR | QILLDAMTNTERALRS | AKTEEAYQRTIKT | 1132, 1133, 1134 |
| ETVEKLT | | QQKL | |
| QNILDRINENEQRVSVLE | QSIQATTSRVDAIEAKV | EIWETNTERSIKA | 1135, 1136, 1137 |
| RTLAQ | KHLEA | VLSIQS | |
| QSIEDSLSTLNTKINKLK | KYISNRIKENTDQIKKLE | AKIETTKKITEELL | 1138, 1139, 1140 |
| KEVESLKREVEEL | ERVTELEA | DRAIK | |
| AKAEHAIKFALSEEKSRS | LEIRQTSKRVESLERRVT | QAIRETQDEVKNL | 1141, 1142, 1143 |
| | QVERDR | NKRINKIVTSI | |
| EIWETNTERSEKKVKSIQ | VTINNMISSNTNEISSLQDRVKHI | | 1144, 1145 |
| S | EDTLAL | | |

Cluster 3

| | | | |
|---|---|---|---|
| REIIRAINIVRKIASEK | RKTLETIEWVEKVIKKQ | RTLLETAEIVTRS | 1146, 1147, 1148 |
| | RS | | |
| AKLKETTERTEKIEKKIK | ALWLEAAKYVKQAREK | RETAKAVSAVK | 1149, 1150, 1151 |
| DS | S | | |
| DELARAATLAKQLITKIK | RKTEKAIRLVLKWLKES | RTLLETAEIVKRS | 1152, 1153, 1154 |
| KS | | | |
| EELAQTARLAKAYLKEL | RDTLKAIEQTKRYLEEL | RKLDKAAEYVEK | 1155, 1156, 1157 |
| KSRS | KKS | S | |
| EYLAQVAEKVDK | RSWDIAAKFVKTVLSNQ | RKLETAAEKLKQT | 1158, 1159, 1160 |
| | S | E | |
| EKQKKINEMATKVT | RKTLEATEIAKKLAEDR | RLMLEAVKIAQSQ | 1161, 1162, 1163 |
| | S | S | |
| EYLKKVAEIVNKIS | LEILKAAKEAKKLIEDLR | RETKEAAESVKQ | 1164, 1165, 1166 |
| | RS | MES | |
| TETKKAIEIALKIS | KELLDAAKAVKKMLEK | RRTLKAIEITLKLL | 1167, 1168, 1169 |
| | EKSS | S | |
| SKLEEALRWVTKVRS | KKLLDAADAVKKMLEK | KKLADAADWVET | 1170, 1171, 1172 |
| | EKSS | VKSS | |
| AKLTKATKYALTVIKQS | KKVLETIRWIETVISRQR | KKTHSAIEWVERL | 1173, 1174, 1175 |
| | SS | VSS | |
| RTLKDTTELTKNLNKKL | ADLKKVAELVKKLMEE | ALLLEAAKYVKK | 1176, 1177, 1178 |
| KKLEEEL | AKKKS | AREKS | |
| RSNKKTKNKVKSIEKQV | TDTMKAARIMKEELKE | ADTKKAAEIAKKL | 1179, 1180, 1181 |
| KEIEKRLEKLERA | KS | AKS | |
| RQIVEVMKEVEELRKRV | AKNAEAAKIAEETKRKD | RKLLEAAEEMEK | 1182, 1183, 1184 |
| ENIEKNL | | MLKTS | |
| QKTRATEEALKKTQKEV | KKLKSAADDVKKAKEK | RKMLEAVEHAKK | 1185, 1186, 1187 |
| TKLKKEIQKLT | S | LKKES | |
| RSNKKTKNKVKSIEKQV | KELKSAAEDVKKAKEK | RKMLEAVEKAKK | 1188, 1189, 1190 |
| KEIEKRLEKLEKA | S | LDKES | |
| REIIRAINIVRKIASEKS | RETKKATENVKTMLTK | RKLEEIARIVEQK | 1191, 1192, 1193 |
| | SKS | KRTEEKRS | |
| RDLDTAAKQVKEMLKE | LELKKAAKAANTDLTK | RDLKKAAEIAKKS | 1194, 1195, 1196 |
| KS | KS | | |
| RETEKTIRQVQEILKKWS | LELKEAAKAANTDLTK | RKTLETIEWVKKV | 1197, 1198, 1199 |
| | KS | IKKQRS | |
| RDTIKVAIIVKELYKKIS | | | 1200 |

### Usage

The universal sequences described here can be used in the following ways. First determine the alignment of the terminal helices, then select the appropriate consensus sequences. Polar positions can be WT polar residues or selected from the most probable residues provided in the positional weights tables, where the designer should ensure that basic and acidic residues are paired along the helix (e.g., basic at position i and acidic at position i+4). Alternatively, a blueprint file can be generated from the positional probability tables. This blueprint is then used as an input for RosettaRemodel which selects identities from the distribution specified.

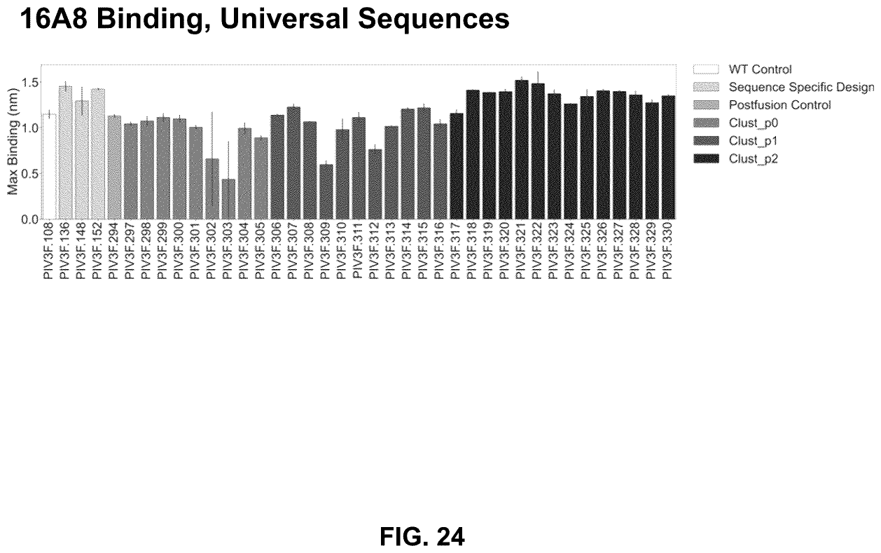

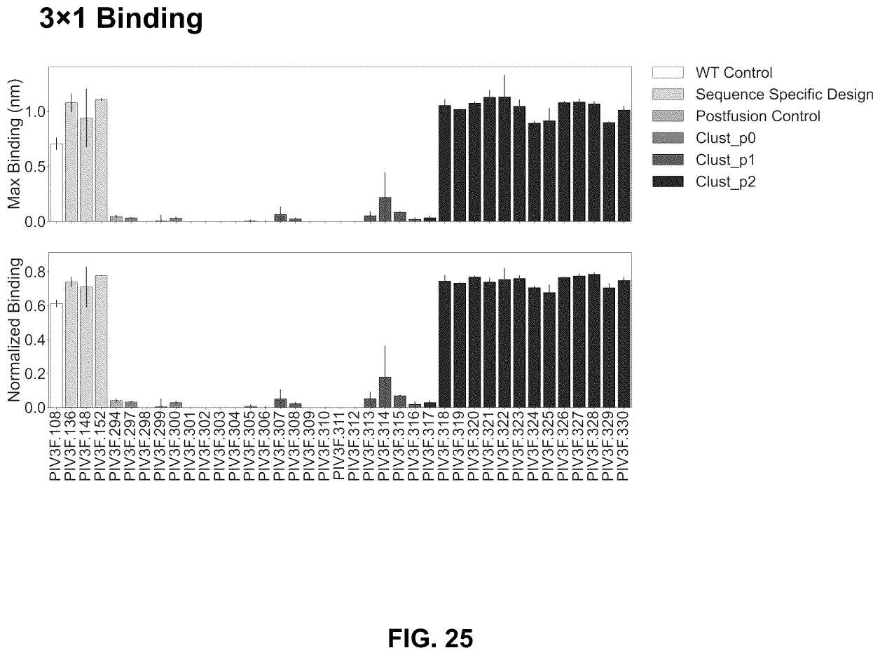

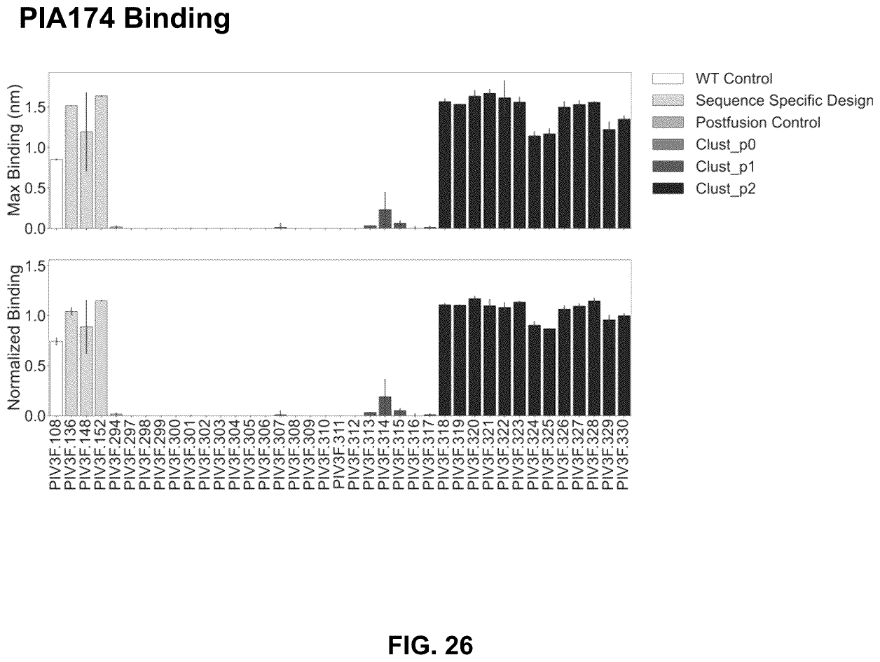

The utility of universal sequences was demonstrated empirically by generating sequences as described above and confirming stabilization of the prefusion conformation of PIV3 F. Because the terminal helices of PIV3 are parallel, sequences were generated from the parallel helix clusters p0, p1, and p2. Nine, eleven, and thirteen sequences were generated from each cluster respectively. These designs were then genetically fused to I53-50AΔcys (Table 26, C-Term-45 to C-Term-78, comprising, respectively, SEQ ID NO: 1201-1234. When expressed and secreted from HEK293 cells, all of the sequences expressed well (FIG. 24). Sequences from cluster p2 successfully stabilized the prefusion conformation, equal to fusion protein specific designs, as measured by binding to 3×1 (FIG. 25) and PIA174 (FIG. 26) by BLI.

TABLE 26
C-terminal alpha-helical segments for PIV3 (clusters p0, p1, and p2)

| Name | C-Term Remode Sequence | Cluster | SEQ ID NO. |
|---|---|---|---|
| C-Term-45 | QKTISDLLEIVEKLIRSL | Clust_p0 | 1201 |
| C-Term-46 | QKTISDLLEIIEKLIRSL | Clust_p0 | 1202 |
| C-Term-47 | QKTISDLLEIVEQLIRSL | Clust_p0 | 1203 |
| C-Term-48 | QKTISDLLEIVENLIRSL | Clust_p0 | 1204 |
| C-Term-49 | QKTISDLLEIIESLLRSL | Clust_p0 | 1205 |
| C-Term-50 | QETIQELLKIVKELIQKL | Clust_p0 | 1206 |
| C-Term-51 | KETIKELLKIIKELIKEL | Clust_p0 | 1207 |
| C-Term-52 | SQTISELLQIVKELLSQL | Clust_p0 | 1208 |
| C-Term-53 | NKTIKELLNIIKSLLEKL | Clust_p0 | 1209 |
| C-Term-54 | VATKKDLEDLIEKLERLLQKLDS | Clust_p1 | 1210 |
| C-Term-55 | VATKKDLEDLIENLERLLQKLDS | Clust_p1 | 1211 |
| C-Term-56 | VTTKKDLEDLIENLKRLLQKLDS | Clust_p1 | 1212 |
| C-Term-57 | VTTKKDLEDLIENLERLLQKLDS | Clust_p1 | 1213 |
| C-Term-58 | VATKKDLEDLIESLKRLLQKLDS | Clust_p1 | 1214 |
| C-Term-59 | VATKKDLEDLIESLERLLQKLDS | Clust_p1 | 1215 |
| C-Term-60 | VTTKKDLEDLIESLKRLLQKLDS | Clust_p1 | 1216 |
| C-Term-61 | VTTKKDLEDLIESLERLLQKLDS | Clust_p1 | 1217 |
| C-Term-62 | VATNKSLQDLIKELKDLLSKLNT | Clust_p1 | 1218 |
| C-Term-63 | VTTKKELKDLIQKLKDLLSKLQT | Clust_p1 | 1219 |
| C-Term-64 | VATKKELKDLITKLEKLLSKLQT | Clust_p1 | 1220 |
| C-Term-65 | VTTKKELKDLIQKLEKLLSKLQT | Clust_p1 | 1221 |
| C-Term-66 | NKVKKDVEELKESVRRLEKKLD | Clust_p2 | 1222 |
| C-Term-67 | NKVKKDVEELKETVRRLEKKLD | Clust_p2 | 1223 |
| C-Term-68 | NKVKKDVEELKENVRRLEKKLD | Clust_p2 | 1224 |
| C-Term-69 | NKVKKDVEELKEQVRRLEKKLD | Clust_p2 | 1225 |
| C-Term-70 | NKVKKDVEELKEEVRRLEKKLD | Clust_p2 | 1226 |
| C-Term-71 | NKVKKDVEELKEDVRRLEKKLD | Clust_p2 | 1227 |
| C-Term-72 | NKVKKDVEELKERVRRLEKKLD | Clust_p2 | 1228 |
| C-Term-73 | NKVKKDVEELKEKVRRLEKKLD | Clust_p2 | 1229 |
| C-Term-74 | NKVKKDVEELKEHVRRLEKKLD | Clust_p2 | 1230 |
| C-Term-75 | NKVKKEVQELKQTVKSLEKELT | Clust_p2 | 1231 |
| C-Term-76 | NKVKKDVNELKQSVKSLEKELT | Clust_p2 | 1232 |
| C-Term-77 | NKVKKEVSELTEKVESLEKKLT | Clust_p2 | 1233 |
| C-Term-78 | NKVKKDVTELSEKVESLEKKLT | Clust_p2 | 1234 |

### Materials and Methods

Protein search: Protein structures were retrieved from the PDB (https://www.rcsb.org/) with the underlying X-ray crystallography or cryo-EM data. Where multiple structures exist, the models with the highest resolution, most complete, and well refined C-terminal domain were selected.

Input preparation: PyMol version 2.5.2 was used to analyze all structural models and generate images. To generate an input for computational design models C3-symmetry axis were aligned to the Z-axis. Where the model was too asymmetric to align, the highest resolution chain was duplicated and aligned to the other chains in the trimer assembly using the PyMol function “super”. An idealized symmetric input was then generated by duplicating the A-chain and rotating it 60 and 120 degrees about the Z-axis. Glycosylated residues were noted and then all heteroatoms stripped from the model. Cleaned and symmetrized models were then relaxed using Rosettarelax (Refs 1 and 2).

Design: Blueprint files were written to generate a gradient between the native structure and the remodeled domain. Generally, the blueprint included a two-residue native sequence that is remodeled using helical constraints; followed by one or more existing helical residues that contribute to hydrophobic contacts at the C-terminus, which are allowed to design; followed by an alpha-helical segment ranging in length from approximately 1-10 amino acids in length, determined empirically by the designer. To determine the appropriate length, designs with progressively longer lengths are generated and scored by calculating the predicted energy in Rosetta Energy Units (REU) of the trimeric assembly (bound state) and again where each protein molecule is translated 1000 Angstroms apart (unbound state). The difference between the bound and unbound state, termed ddG, is an estimate of the interface strength. A plot of the average ddG as a function of length reveals a minimum length where designs are, on average, >10 REU better than the WT, and a maximum length where increasing length no longer improves ddG. The blueprint is set up to allow repacking in the two residues preceding the de novo designed region. Where structural data supports inclusion, the following residues in the C-terminal domain are allowed to repack with sequence design. This region is selected based on the criteria that the experimental data supports the model, and that there are no native contacts with the rest of the ectodomain. If there is a glycosylation site it is constrained to the WT sequence. Remodel results were filtered by ddG calculations from the design model directly, and manually reverted to remove any introduced glycosylation sites, exposed hydrophobic residues, or buried polar residues. The resulting model were relaxed and then ddG's were again calculated. In some cases all remodel lengths were far superior to the WT. In that case, an minimum remodel length was selected based on a reasonable interface size containing at least 3 helical turns. Alternatively, remodeling was performed using RFdiffusion (Ref. 3). Relaxed structures used as input for remodel were also used as input for RFdiffusion, except that only the C-terminal helices were used as input for diffusion. This significantly reduces computational time. Positions with WT sequence identities from Rosetta Remodel were likewise preserved for diffusion. Unlike with Rosetta Remodel all structural data of subsequent residues was ignored. This is a limitation of RFdiffusion, not a scientific constraint placed on the design problem. Intra-protomer and inter-protomer weights were both set to 1, with a quadratic guide decay and a guide scale of 2. C-terminal lengths varied between 12 and 31 residues depending on the source virus, and 15 remodeled helices were generated for each length. Sequences were generated using Protein MPNN, with a sampling temperature of 0.4 and a negative bias against C (−10), and F, G, P, W, and Y (−1). 100 sequences were generated for each remodeled domain. The top 2% based on MPNN sample score were selected for computational characterization. Designs were analyzed based on the following criteria: 1) ColabFold validates the design generated by Rossetta or RFdiffusion by predicting an ordered terminal helix consistent with design model; 2) decrease in ddG by 5-15 Rosetta Energy Unit (REU); 3) Design has a well-packed hydrophobic core without extraneous elements (i.e. helical segments with no interprotomer hydrophobic packing).

Small-Scale Transfection: A variety of RSV/B designed were screened for expression, antigenicity and thermal stability via 96 deep well transfections. Expi293 cells in log phase growth were counted and seeded at 2.5×10⁶ cells/ml. Cells were incubated overnight at 36° C. with shaking (120 rpm). The next day the cells were counted and diluted to 3×106 cells/ml in 0.6 ml per well. Cells were transiently transfected as follows. A 5× master mix of 1000 μg plasmid DNA was diluted to 35 ml final volume with OptiMEM™ and gently mixed in a separate 96 deep well plate. A 5× master mix of Transporter 5 transfection reagent was diluted to a final volume of 35 ml with OptiMEM™ and gently mixed. The Diluted Transporter 5 was added to the diluted DNA, mixed, incubated at room temperature for 10 minutes and then 42 μl was added dropwise to each well while gently shaking plate. Cells were placed back in the incubator, shaking at 1050 rpm in for 4 days.

Biolayer Interferometry: Antibodies 16A8 (ATUM), AM14, 4D7, D25, and Palivizumab (Creative Biolabs) were normalized in concentration to 10 μg/mL in BLI assay buffer (PBS, 0.5% BSA, 0.05% Tween 20, pH 7.4) in a sufficient volume to load 80 μL per well of a black 384-well microplate (Thermo Scientific, 460518). Briefly, on an Octet rh16 instrument, Protein G biosensors (Sartorius, 18-5082) were dipped into assay buffer for 60 seconds to achieve a baseline. Next, the biosensors were dipped into each antibody for 60 s in order to immobilize the antibodies present but without reaching saturation, followed by an additional baseline step. The immobilized antibodies were allowed to associate with 80 μL of RSV/B supernatant for 120 seconds, and then the biosensors were dipped back into assay buffer for 120 seconds to observe any possible dissociation. 16A8 is a monoclonal antibody that recognizes I53-50A and was used to estimate relative expression levels. AM14, D25, 4D7, and Palivizumab are specific to RSV F protein.

Large-Scale Transfection: Based on the data from the 96 deep well screen, a subset of constructs were expressed transiently at the 1-liter scale. Expi293 cells in log phase growth were counted and seeded in 220 ml at 2.5×106 cells/ml in each of four 1 L flasks (total volume 880 ml). Cells were incubated overnight at 36° C. with shaking (120 rpm). The next day the cells were counted and diluted to 3×10⁶ cells/ml in 232.5 ml per 1 L flask. Cells were transiently transfected as follows. 1000 μg plasmid DNA was diluted to 35 ml final volume with OptiMEM™ and gently mixed. 2.5 ml of Transporter 5 transfection reagent was diluted to a final volume of 35 ml with OptiMEM™ and gently mixed. The Diluted Transporter 5 was added to the diluted DNA, mixed, incubated at room temperature for 10 minutes and then 17.5 ml added dropwise to each 1 L flask while gently swirling the flask. Cells were placed back in the incubator, shaking for 4 days. A temperature shift to 33° C. was incorporated the day after transfection to increase protein yields.

Immobilized Metal Affinity Chromatography: Four mL of Ni²⁺ IMAC resin (Indigo, Cube Biotech cat #75103) per one liter of cell supernatant was equilibrated into IMAC wash buffer (20 mM Tris pH 8.0, 300 mM NaCl, 30 mM imidazole). Tris pH 8.0 was added at 50 mM per liter and NaCl was added to 300 mM per liter. Cell supernatants were batch bound overnight at 4° C. with stir bar agitation. After overnight incubation, cell supernatants were transferred to gravity columns and flow through was collected. Resin was then washed with 40 mL of IMAC wash buffer and flow through buffer was collected. Columns were sealed and eight mL of IMAC elution buffer (20 mM Tris pH 8.0, 300 mM NaCl, 500 mM imidazole) was added to each column and allowed to incubate for ten minutes. Column was unstopped and elution flow through was collected. Elution incubation was repeated twice. SDS-PAGE gel was done to confirm protein of interest was captured in elution fractions.

Differential Scanning Fluorimetery: Nano-DSF thermal ramp was used to estimate the Tonset and melting temperature (Tm) of antigen samples using SYPRO Orange Protein Gel Stain (Invitrogen) on an UNcle Nano-DSF (UNchained Laboratories). Antigen samples samples were normalized to a concentration of ˜1 mg/mL (or 0.3-0.45 mg/mL for low expressing constructs) by adding antigen samples to PCR tubes then adding buffer (20 mM Tris pH 7.4, 250 mM NaCl, 4% sucrose) to a final volume of 31.5 μL. SYPRO was diluted from 5000× to a 200× working stock solution by adding 4 μL of SYPRO to 96 μL of buffer. Then, 3.5 μL of the 200× stock solution was added to each PCR tube to bring SYPRO to 20×. Antigen sample dilutions with SYPRO were applied to quartz capillary cassettes (UNi, UNchained Laboratories) in triplicate and placed in the UNcle. Data were collected using a temperature ramp from 15° C. to 95° C. (holding samples at 15° C. for 300 seconds prior to data collection), collecting data at 1° C. increments. Improved Tonset and Tm were observed for all constructs compared to RSV/A.03 and RSV/B.002.

Accelerated Storage: Binding of RSV F specific antibodies were assessed on trimeric antigen-I53-50AΔcys fusion proteins following incubation of the antigen samples at 4° C. or 40° C. for 7 days. Antibodies were normalized in concentration to 10 μg/mL in BLI assay buffer (PBS, 0.5% BSA, 0.05% Tween 20, pH 7.4) in a sufficient volume to load 80 μL per well of a black 384-well microplate (Thermo Scientific, 460518). Briefly, on an Octet rh16 instrument, Protein G biosensors (Sartorius, 18-5082) were dipped into assay buffer for 60 seconds to achieve a baseline. Next, the biosensors were dipped into each antibody for 60 seconds in order to immobilize the antibodies present but without reaching saturation, followed by an additional baseline step. The immobilized antibodies were allowed to associate with 80 μL of purified RSV antigen (normalized in concentration to 10 μg/mL) that was incubated at either 4° C. and 40° C. for 7 days for 120 seconds, and then the biosensors were dipped back into assay buffer for 120 seconds to observe any possible dissociation. The new designs have higher AM14 binding and lower 4D7 binding than the controls (RSV/A.03 and RSV/B.002) indicating less postfusion character and a more compact trimer. Decreased D25 and AM14 binding and increased 4D7 binding was observed for RSV/A.03 and RSV/B.002 following 7 days at 40° C. while binding of all Abs was unaffected by 7 days at 40° C. for the other constructs tested.

Assembly: Molar concentrations for RSV/B or RSV/A trimers fused to I53-50AΔcys and I53-50B (second component, using the sequence of I53-50B.4PosT1, SEQ ID NO:46) were determined using UV-Vis spectroscopy. Absorbance values at 280 nm were collected and divided by calculated molar extinction coefficients (ExPASy). The assembly reaction to produce RSVB antigen-bearing nanostructures was performed in vitro with the addition of components as follows: RSV F trimers fused to I53-50AΔcys were added to PCR tubes in 1.5× molar excess of I53-50B, then assembly buffer (20 mM Tris pH 7.4, 250 mM NaCl, 4% sucrose) was added to the sample in PCR tubes, and finally I53-50B was added to the reaction to bring the assemblies to a final concentration of 7.5 μM and a final volume of 47.5 μL. The reaction was incubated at ambient temperature for ˜30 minutes with gentle rocking prior to subsequent measurement by dynamic light scattering (DLS). All constructs were assembly competent under the conditions tested. Prior to nsEM analysis or immunogenicity studies, assembled nanostructures were further purified by size exclusion chromatography over a Superose 6 Increase 10/300 GL column into 20 mM Tris pH 7.4, 250 mM NaCl, 4% sucrose.

VLPs was performed in vitro with the addition of components as follows: CompAs were added to PCR tubes in 1.5× molar excess of CompB, then assembly buffer (20 mM Tris pH 7.4, 250 mM NaCl, 4% sucrose) was added to the CompA in PCR tubes, and finally CompB was added to the reaction to bring the assemblies to a final concentration of 7.5 μM and a final volume of 47.5 μL. The reaction was incubated at ambient temperature for ˜30 minutes with gentle rocking prior to subsequent measurement by dynamic light scattering (DLS). All constructs were assembly competent under the conditions tested.

Dynamic Light Scattering: Dynamic Light Scattering (DLS) was used to measure hydrodynamic diameter (Dh) and polydispersity (% Pd) of nanostructure assemblies on an UNcle Nano-DSF (UNchained Laboratories). The set up included increased viscosity due to 4% sucrose in the buffer that was accounted for by the UNcle Client Software in Dh measurements. RSV/B nanostructure assemblies were applied to quartz capillary cassettes (UNi, UNchained Laboratories) in triplicates and measured using the laser autoattenuation with 10 acquisitions per sample and 5 seconds per acquisition. Data were collected at 22° C. and all tested constructs resulted in monodisperse nanostructures of the expected size.

Immunogenicity studies: Two immunogenicity studies were undertaken in 6-8-week-old, female BALB/c mice to evaluate the neutralizing antibody response elicited by RSV/A and RSV/B designs. In order to evaluate nanostructures based on RSV/A designs RSV/A.03, RSV/A.013, and RSV/A.023, mice were immunized with either 0.01 μg, 1 μg, or 5 μg of nanostructure protein. The 0.01 μg dose was adjuvanted with oil-in-water emulsion, AddaVax, while the 1 μg and 5 μg doses were unadjuvanted. Mice were immunized on days 0 and 21 before being sacrificed on Day 35. Serum collected on Day 35 was used to perform a neutralization assay with the RSV/A Tracy strain. Nanostructures displaying RSV/B designs RSV/B.002, RSV/B.093, RSV/B.195, RSV/B.160, and RSV/B.171 were similarly evaluated. Mice were immunized on days 0 and 21 with either a 0.02 μg or 0.1 μg dose of nanostructure sample adjuvanted with AddaVax. Serum samples collected during the terminal bleed on Day 35 were used to perform a neutralization assay with RSV/B strain 18537. Both the RSV/A and RSV/B neutralization assays were performed in Hep-2 cells. Two-fold serial dilutions of serum samples were prepared in 96-well plates. An equal volume of virus was added to each dilution and incubated for 1.5 hours before the addition of Hep-2 cells. Plates were incubated for 6-8 days before being fixed and stained with 10% neutral formalin and 0.01% crystal violet. Neutralizing antibody titers were defined as the final dilution at which there was a 50% reduction in viral cytopathic effect. Statistically significant differences between groups immunized with different designs at the same dose were determined by one-way ANOVA.

Cryo-electron microscopy: IMAC-purified trimeric RSV/A.023 sample was further purified over a Superdex 200 Increase 10/300 GL column unto 20 mM Tris pH 7.4, 250 mM NaCl, and further concentrated to 0.88 mg/mL prior to grid preparation. The concentrated sample was next frozen using a Quantifoil R 1.2/1.3 AU 300 holey grid. Data collection was performed using a Glacios 200ke V microscope equipped with a Falcon IV detector (0.91 Å/pixel). A C3-symmetric model of RSVA023 was rebuilt from PDB 4MMU using COOT. The final atomic structure was refined in Phenix and validated using MolProbity and the half-map cross validation method. Structural analysis was performed using COOT, Chimera and PyMol.

Electron Microscopy: For negative stain electron microscopy (nsEM), RSV F protein-nanostructure pre- and post-freeze samples were diluted to 75 μg/mL in 20 mM Tris pH 8.0, 150 mM NaCl, 5% Glycerol and 3 μL of sample was applied to the carbon side of two glow-discharged (Pelco EasiGLOW) thick carbon copper 400 mesh grids (EMS, CF400-Cu-TH). Samples were incubated on the grids for ˜1 minute, then blotted away using grade 1 filter paper (Whatman). Immediately, 3 μL of 0.75% UF stain was applied to to the carbon side of the girds and incubated for ˜1 minute. The stain was blotted away using filter paper and the application of stain and blotting was repeated 2 more times. The grids were allowed to air dry for 5 minutes prior to imaging on a Talos L120C electron microscope at 57K magnification, Gatan camera. Micrographs shows correct self-assembly of monodisperse nanostructures.

### REFERENCES

1. Khatib F, Cooper S, Tyka M D, Xu K, Makedon I, Popovic Z, Baker D, and Players F. (2011). Algorithm discovery by protein folding game players. Proc Natl Acad Sci USA 108 (47): 18949-53. doi: 10.1073/pnas.1115898108.
2. Maguire J B, Haddox H K, Strickland D, Halabiya S F, Coventry B, Griffin J R, Pulavarti S V S R K, Cummins M, Thieker D F, Klavins E, Szyperski T, DiMaio F, Baker D, and Kuhlman B. (2020). Perturbing the energy landscape for improved packing during computational protein design. Proteins “in press”. doi: 10.1002/prot.26030.10966648: Xtal structure of tetrabrachion tetramerization domain
3. Watson, J. L., Juergens, D., Bennett, N. R. et al. De novo design of protein structure and function with RFdiffusion. Nature (2023). doi: 10.1038/s41586-023-06415-8
4. Protein DataBank code 4GIP
5. Protein DataBank code 8DG8
6. Protein DataBank code 7UP9
7. Protein DataBank code 5WB0
8. Protein DataBank code 4MMU
9. Protein DataBank code 7LAB
10. Che, Y et al. Rational design of a highly immunogenic prefusion-stabilized F glycoprotein antigen for a respiratory syncytial virus vaccine. Sci. Transl. Med. (2023) doi: 10.1126/scitranslmed.ade6422
11. Stewart-Jones et al. A Cysteine Zipper Stabilizes a Pre-Fusion F Glycoprotein Vaccine for Respiratory Syncytial Virus. PloS One (2015). doi: 10.1371/journal.pone.0128779
12. Stetefeld, J et al., Crystal structure of a naturally occurring parallel right-handed coiled coil tetramer. Nat. Struct. Biol. (2000). doi: 10.1038/79006.

### Abbreviations

RSV Respiratory Syncytial Virus
REU Rosetta Energy Unit
PDB Protein Data Bank
EDTA ethylenediaminetetraacetic acid
DLS Dynamic Light Scattering
nsEM negative-stain electron microscopy
UNcle UNchained Laboratories
UNi UNchained Laboratories

### INCORPORATION BY REFERENCE

The entire disclosure of each of the patent and scientific documents referred to herein is incorporated by reference for all purposes.

The reference in this specification to any prior publication (or information derived from it), or to any matter which is known, is not, and should not be taken as an acknowledgment or admission or any form of suggestion that prior publication (or information derived from it) or known matter forms part of the common general knowledge in the field of endeavour to which this specification relates.

### EQUIVALENTS

The invention may be embodied in other specific forms without departing from the spirit or essential characteristics thereof. The foregoing embodiments are therefore to be considered in all respects illustrative rather than limiting on the invention described herein. The scope of the invention is thus indicated by the appended claims rather than by the foregoing description, and all changes that come within the meaning and range of equivalency of the claims are intended to be embraced therein.

1. A recombinant polypeptide, comprising an engineered ectodomain of a trimeric viral protein, wherein the ectodomain comprises:
a C-terminal helix forming segment comprising one or more amino acid substitutions, relative to a native reference sequence of the viral protein, selected such that the segment forms a stable alpha-helical homotrimer.

2. The recombinant polypeptide of claim 1, wherein the C-terminal helix forming segment has improved hydrophobic packing compared to the native reference sequence.

3.-4. (canceled)

5. The recombinant polypeptide of claim 1, wherein the C-terminal helix forming segment comprises a polypeptide sequence according to any one of:
(SEQ ID NO: 566)
LXXTIXXLLXIXXXLXXXL
(SEQ ID NO: 567)
LVXTXKXLXDLIXXLXXLLXKLXX
(SEQ ID NO: 568)
LNKVKKXVXXLXXXVXXLEKXLX
(SEQ ID NO: 569)
EKIXXAIKKAXKL
(SEQ ID NO: 570)
EXIXKAIKXLXXXXX
(SEQ ID NO: 571)
XKXXEXXXXVXXXXXXXXX
(SEQ ID NO: 572)
XXLKKAAXIXKKXLKXX.

6.-9. (canceled)

10. The recombinant polypeptide of claim 1, wherein the native reference sequence of the viral protein is any one of SEQ ID NOs: 1, 104, 327, 382, 459, and 499.

11. The recombinant polypeptide of claim 1, comprising an engineered ectodomain of a hMPV fusion (F) protein, wherein the ectodomain comprises a C-terminal helix-forming segment, between about residue 470 and about residue 500 relative to SEQ ID NO: 104, comprising one or more amino acid substitutions selected such that the segment forms a stable alpha-helical homotrimer.

12. The polypeptide of claim 11, wherein the C-terminal helix-forming segment comprises substitutions relative to the reference sequence SEQ ID NO: 104 at two or more, three or more, or four or more residues that generate hydrophobic contacts between the segments in the alpha-helical homotrimer.

13.-14. (canceled)

15. The polypeptide of claim 11, wherein the segment comprises:
(1) an amino acid substitution at position Q471 relative to SEQ ID NO: 104, wherein Q is substituted with any one of A, D, E, I, Q, R, S, T;
(2) an amino acid substitution at position A472 relative to SEQ ID NO: 104, wherein A is substituted with any one of A, D, E, I, K, R, S, T, Y;
(3) an amino acid substitution at position L473 relative to SEQ ID NO: 104, wherein Lis substituted with any one of A, I, L, M, Q, S, T, W;
(4) an amino acid substitution at position V474 relative to SEQ ID NO: 104, wherein Vis substituted with any one of A, D, E, I, K, L, N, Q, S, T;
(5) an amino acid substitution at position D475 relative to SEQ ID NO: 104, wherein D is substituted with any one of A, D, E, H, K, N, Q, R, S, T;
(6) an amino acid substitution at position Q476 relative to SEQ ID NO: 104, wherein Q is substituted with any one of A, D, E, H, I, K, L, M, N, Q, T, V;
(7) an amino acid substitution at position S477 relative to SEQ ID NO: 104, wherein S is substituted with any one of A, E, I, K, L, M, N, Q, R, S, T, V;
(8) an amino acid substitution at position N478 relative to SEQ ID NO: 104, wherein Nis substituted with any one of A, D, E, K, N, Q, R, S, T;
(9) an amino acid substitution at position R479 relative to SEQ ID NO: 104, wherein R is substituted with any one of A, D, E, F, I, K, L, M, N, Q, R, S, T, W, Y;
(10) an amino acid substitution at position I480 relative to SEQ ID NO: 104, wherein I is substituted with any one of A, I, L, M, R, S, T, V;
(11) an amino acid substitution at position L481 relative to SEQ ID NO: 104, wherein L is substituted with any one of D, E, I, K, L, M, N, Q, R, S, T;
(12) an amino acid substitution at position S482 relative to SEQ ID NO: 104, wherein S is substituted with any one of A, D, E, K, Q, R, S, T;
(13) an amino acid substitution at position S483 relative to SEQ ID NO: 104, wherein S is substituted with any one of A, D, E, F, H, I, K, L, M, N, Q, R, S, T, V, W, Y;
(14) an amino acid substitution at position A484 relative to SEQ ID NO: 104, wherein A is substituted with any one of A, D, E, I, K, L, M, R, S, T, V, Y;
(15) an amino acid substitution at position E485 relative to SEQ ID NO: 104, wherein E is substituted with any one of D, E, G, K, L, Q, R, S, T;
(16) an amino acid substitution at position K486 relative to SEQ ID NO: 104, wherein K is substituted with any one of A, E, I, K, L, Q, R, S, T;
(17) an amino acid substitution at position G487 relative to SEQ ID NO: 104, wherein Gis substituted with any one of A, E, I, K, L, R, S, T, V;
(18) an amino acid substitution at position N488 relative to SEQ ID NO: 104, wherein Nis substituted with any one of E, I, K, L, N, Q, R, S;
(19) an amino acid substitution at position T489 relative to SEQ ID NO: 104, wherein Tis substituted with any one of A, D, E, K, S; and/or
(20) any combination of (1)-(19).

16. The polypeptide of claim 11, wherein the segment comprises a polypeptide sequence of SEQ ID NO: 182 to SEQ ID NO: 326 or SEQ ID NO: 555 to SEQ ID NO: 565, or a polypeptide sequence having between 1 and 5 amino acid substitutions thereto.

17.-21. (canceled)

22. The recombinant polypeptide of claim 1, comprising an engineered ectodomain of a PIV3 fusion (F) protein, wherein the ectodomain comprises a C-terminal helix-forming segment, between about residue 460 and about residue 490-relative to SEQ ID NO: 327, comprising one or more amino acid substitutions selected such that the segment forms a stable alpha-helical homotrimer.

23.-31. (canceled)

32. The recombinant polypeptide of claim 1, comprising an engineered ectodomain of a PIV5 fusion (F) protein, wherein the ectodomain comprises a C-terminal helix-forming segment, between about residue 460 and about residue 490 relative to SEQ ID NO: 382, comprising one or more amino acid substitutions selected such that the segment forms a stable alpha-helical homotrimer.

33.-37. (canceled)

38. The recombinant polypeptide of claim 1, comprising an engineered ectodomain of a SARS-CoV2 spike(S) protein, wherein the ectodomain comprises a C-terminal helix-forming segment, between about residue 1140 and about residue 1170-relative to SEQ ID NO: 459, comprising one or more amino acid substitutions selected such that the segment forms a stable alpha-helical homotrimer.

39.-47. (canceled)

48. The recombinant polypeptide of claim 1, comprising an engineered ectodomain of a Nipah fusion (F) protein, wherein the ectodomain comprises a C-terminal helix-forming segment, between about residue 460 and about residue 490-relative to SEQ ID NO: 499, comprising one or more amino acid substitutions selected such that the segment forms a stable alpha-helical homotrimer.

49.-81. (canceled)

82. A trimeric protein complex comprising a recombinant polypeptide according to claim 1.

83.-85. (canceled)

86. A protein nanostructure comprising a trimeric component comprising a recombinant polypeptide according to claim 1.

87. The protein nanostructure of claim 86, wherein the nanostructure is a two-component nanostructure comprising the first, trimeric component and a second, pentameric component, wherein the first trimeric component further comprises an I53-50A polypeptide.

88.-93. (canceled)

94. The protein nanostructure of claim 86, wherein the trimeric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one of the sequences of SEQ ID NO: 76 to SEQ ID NO: 103 or to any one of the sequences of SEQ ID NO: 76 to SEQ ID NO: 103 without the underlined and/or bold/italicized polypeptide sequences.

95. The protein nanostructure of claim 87, wherein the pentameric component comprises a polypeptide sequence at least 70%, at least 80%, at least 90%, at least 91%, at least 92%, at least 93%, at least 94%, at least 95%, at least 96%, at least 97%, at least 98%, at least 99%, or 100% identical to any one or more of SEQ ID NOs: 20, 44, 45, 52, 71, 73, 74.

96. A pharmaceutical composition comprising a nanostructure according to claim 86.

97.-110. (canceled)

111. A polynucleotide encoding the recombinant polypeptide of claim 1.

112.-113. (canceled)

114. A method of vaccinating a subject, generating an immune response in subject, and/or treating or preventing a viral infection in a subject, the method comprising administering to the subject the pharmaceutical composition of claim 96.

115.-191. (canceled)
